(19) United States
(12) Patent Application Publication
(10) Pub. No.: US 2026/0306502 A1
(43) Pub. Date: Oct. 8, 2026

(54) MITOCHONDRIAL UNCOUPLERS FOR TREATMENT OF METABOLIC DISEASES AND CANCER

(71) Applicants: Mito Biopharma, LLC, North Brunswick Township, NJ (US); Rutgers, The State University of New Jersey, New Brunswick, NJ (US)

(72) Inventors: Shengkan JIN, Belle Mead, NJ (US); David J. AUGERI, Princeton, NJ (US); Bin CAO, Skillman, NJ (US); Hanlin TAO, Branchburg, NJ (US); Nigel LIVERTON, Harleysville, NJ (US); Casey MCCOMAS, Phoenixville, PA (US)

(73) Assignees: Mito Biopharma, LLC, North Brunswick Township, NJ (US); Rutgers, The State University of New Jersey, New Brunswick, NJ (US)

(21) Appl. No.: 19/109,184

(22) PCT Filed: Aug. 28, 2023
(86) PCT No.: PCT/US2023/073006

§ 371 Date: Mar. 6, 2025

Related U.S. Application Data
(60) Provisional application No. 63/374,927, filed on Sep. 8, 2022.

(51) Int. Cl. A61K 31/5377; A61K 31/428; A61K 31/496; A61P 35/00; A61P 37/06; C07D 277/82; C07D 417/12

(52) U.S. Cl. CPC A61K 31/5377; A61K 31/428; A61K 31/496; A61P 35/00; A61P 37/06; C07D 277/82; C07D 417/12

(57) ABSTRACT

The present application discloses a distinct class of mitochondrial uncoupling compounds as well as conventional mitochondrial uncoupling compounds with drastically improved pharmacokinetic properties, pharmaceutical compositions containing the compounds and/or the prodrugs and methods of using the compounds, prodrugs and pharmaceutical compositions in treating diseases related to mitochondrial dysfunctions or diseases benefiting from modulating mitochondrial activities, including diabetes, non-alcoholic fatty liver disease (NAFLD), non-alcoholic steatohepatitis (NASH), mitochondrial genetic diseases, neurodegenerative diseases, cancer, autoimmune diseases, and infectious diseases.

### CROSS REFERENCE TO RELATED APPLICATIONS

This application claims priority to U.S. Provisional Application No. 63/374,927, filed on Sep. 8, 2022, which is hereby incorporated by reference in its entirety.

### SUMMARY

The present disclosure relates to new mitochondrial uncoupling compounds that retain mitochondrial membrane potential as well as new mitochondrial uncoupling compounds with unexpectedly improved pharmacokinetic properties. Various embodiments described herein provide benzamide compounds, prodrugs of the compounds, pharmaceutical compositions containing the compounds and/or the prodrugs and methods of using the compounds, prodrugs and pharmaceutical compositions in the treatment of diseases related to metabolism including diabetes, Non-Alcoholic Fatty Liver Disease (NAFLD), Non-Alcoholic Steathohepatitis (NASH), cancer, autoimmune diseases, dyslipodemia, and infectious diseases.

Some embodiments of the present disclosure are directed to a compound of Formula A:

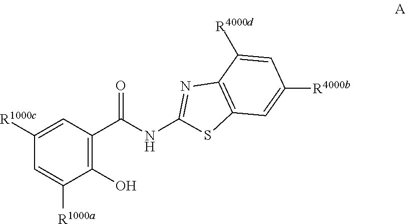

wherein R^{1000a} of Formula A is selected from the group consisting of
   —CH₃, —CH₂CH₃, —C₁-C₆ alkyl, —C₃-C₆ cycloalkyl, —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)NHCO₂CH₃,

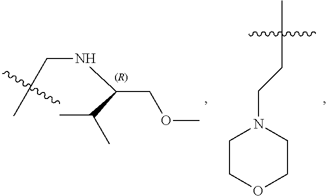

—(CH₂)_{r′}NR^{5000A}R^{5000B}, —(CH₂)ᵣR⁶⁰⁰⁰, and C(O)N(CH₂CH₂OCH₃)₂;
each of substituents R^{5000A} and R^{5000B} is independently selected from the group consisting of —C₁-C₆ alkyl; and —C₁-C₆ alkyl substituted with one or more groups selected from —C₁-C₆ alkoxy, and —O(CH₂)₂OCH₃; alternatively R^{5000A} and R^{5000B} together with the nitrogen to which they are attached, form a 4 to 8-membered heterocyclyl optionally substituted with one or more substituents independently selected from the group consisting of oxo, cyano, hydroxyl, alkoxy, acylamino, carboxyamido, —SO₂CH₃, —CF₃, C₁-C₆ alkyl, halo, and acyl;
R⁶⁰⁰⁰ is selected from the group consisting of a 5 to 6-membered heterocyclyl, pyridinyl and thiazolyl;
r′ is an integer selected the group consisting of 1, 2 and 3;
r is an integer selected from the group consisting of 0, 1, 2 and 3;
R^{1000C} of Formula I is selected from the group consisting of chloro, fluoro, iodo, and bromo;
each of R^{4000b} and R^{4000d} is independently selected from the group consisting of Y¹⁰⁰⁰ and Z¹⁰⁰⁰, provided that when R^{4000b} is Y¹⁰⁰⁰, R^{4000d} is Z¹⁰⁰⁰ and when R^{4000b} is Z¹⁰⁰⁰, R^{4000d} is
Y¹⁰⁰⁰ is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl;
Z¹⁰⁰⁰ is selected from the group consisting of H, —CH₂OCH₃, —CH₂OCH₂CH₃, —CH₂O(CH₂)₂OH, —CH₂O(CH₂)₂OCH₃, —CH₂O(CH₂)₂N(CH₃)₂, —CH₂O(CH₂)₂NHSO₂CH₃, —(CH₂)O(CH₂)₂NR^{2000A}R^{2000B}, —(CH₂)ₛR³⁰⁰⁰, —CH₂OCH₂Ar¹, OCH₃CH₂NHC(O)CH₂CH₃, —CH₂NHC(O)CH₂OCH₃, —CH₂NHSO₂CH₃, —(CH₂)_{t′}NR^{7000A}R^{7000B}, and —(CH₂)ₜR⁸⁰⁰⁰;
R^{2000A} and R^{2000B} together with the nitrogen to which they are attached, form a 4 to 8-membered heterocyclic ring optionally substituted with one or more methyl groups;
R³⁰⁰⁰ is a 5 to 6-membered heterocyclic ring;
Ar¹ is a 5 to 6-membered aryl or heteroaryl group optionally substituted with one or more substituents, independently selected from C₁-C₆ alkyl, halo, hydroxyl and alkoxy;
each of R^{7000A} and R^{7000B} is independently selected from C₁-C₆ alkyl; alternatively, R^{7000A} and R^{7000B} together with the nitrogen to which they are attached, form a 4 to 8-membered heterocyclyl optionally substituted with one or more substituents independently selected from C₁-C₆ alkyl;
R⁸⁰⁰⁰ is selected from the group consisting of a 5 to 6-membered heterocyclic ring optionally substituted with methyl;
s is an integer selected from the group consisting of 0, 1, 2 and 3;
t′ is an integer selected from the group consisting of 1, 2 and 3;
t is an integer selected from the group consisting of 0, 1, 2 and 3;
   with the proviso that
R^{5000A} and R^{5000B} are not both C₁-C₆ alkyl; and
when Z¹⁰⁰⁰ is H; R^{1000a} is not C₁-C₆ alkyl, —C₃-C₆ cycloalkyl, CH₃ or CH₂CH₃, or a pharmaceutically acceptable salt, solvate, or prodrug thereof.

Some embodiments of the present disclosure are directed to a compound of Formula I:

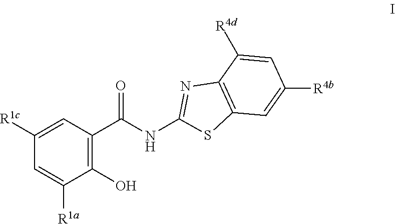

wherein:
R^{1a} is selected from the group consisting of —CH₃, —CH₂CH₃, —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)NHCO₂CH₃,

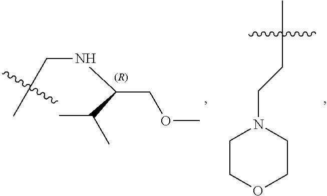

—(CH₂)_{m′}NR^{5A}R^{5B}, (CH₂)ₘR⁶, and C(O)N(CH₂CH₂OCH₃)₂;
each of R^{5A} and R^{5B} is independently selected from the group consisting of —C₁-C₆ alkyl; and —C₁-C₆ alkyl substituted with one or more groups selected from —C₁-C₆ alkoxy, and —O(CH₂)₂OCH₃; alternatively R^{5A} and R^{5B} together with the nitrogen to which they are attached, form a 4 to 8-membered heterocyclyl optionally substituted with one or more substituents, independently selected from the group consisting of oxo, cyano, hydroxyl, alkoxy, acylamino, carboxyamido, —SO₂CH₃, —CF₃, C₁-C₆ alkyl, halo, and acyl;
R⁶ is selected from the group consisting of a 5 to 6-membered heterocyclyl, pyridinyl and thiazolyl;
m′ is an integer selected the group consisting of 1, 2 and 3;
m is an integer selected the group consisting of 0, 1, 2 and 3;
R^{1c} is selected from the group consisting of chloro, fluoro, iodo, and bromo; each of R^{4b} and R^{4d} is independently selected from the group consisting of Y and Z, provided that when R^{4b} is Y, R^{4d} is Z and when R^{4b} is Z, R^{4d} is Y;
Y is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl;
Z is selected from the group consisting of —CH₂OH, —CH₂OCH₂CH₃, —CH₂OCH₃, —CH₂O(CH₂)₂OH, —CH₂O(CH₂)₂OCH₃, —CH₂O(CH₂)₂NHCH₃, —CH₂O(CH₂)₂N(CH₃)₂, —CH₂O(CH₂)₂NHSO₂CH₃, —(CH₂)O(CH₂)₂NR^{2A}R^{2B}, —(CH₂)ₙR³, —CH₂OCH₂Ar, and —OCH₃;
R^{2A} and R^{2B} together with the nitrogen to which they are attached, form a 4 to 8-membered heterocyclic ring optionally substituted with one or more methyl groups;
R³ is selected from the group consisting of a 5 to 6-membered heterocyclic ring and phenoxy;
n is an integer selected from the group consisting of 0 1, 2 and 3; and
Ar is a 5-6 membered aryl or heteroaryl group optionally substituted with one or more substituents, independently selected from the group consisting of —C₁-C₆ alkyl, halo, hydroxy, and alkoxy;
   or a pharmaceutically acceptable salt, solvate, or prodrug thereof.

Some embodiments of the present disclosure are directed to a compound of Formula II:

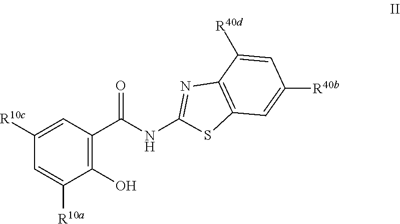

wherein:
R^{10a} is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)NHCO₂CH₃,

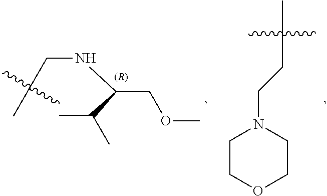

—(CH₂)_{o′}NR^{50A}R^{50B}, —(CH₂)ₒR⁶⁰, and C(O)N(CH₂CH₂OCH₃)₂;
each of R^{50A} and R^{50B} is independently selected from the group consisting of —C₁-C₆ alkyl; and —C₁-C₆ alkyl substituted with one or more groups selected from —C₁-C₆ alkoxy, and —O(CH₂)₂OCH₃; alternatively R^{50A} and R^{50B} together with the nitrogen to which they are attached, form a 4 to 8-membered heterocyclyl optionally substituted with one or more substituents, independently selected from the group consisting of oxo, cyano, hydroxyl, alkoxy, acylamino, carboxyamido, —SO₂CH₃, —CF₃, C₁-C₆ alkyl, halo, and acyl;
R⁶⁰ is selected from the group consisting of 5 to 6-membered heterocyclyl, pyridinyl and thiazolyl;
o′ is an integer selected from the group consisting of 1, 2 and 3;
o is an integer selected from the group consisting of 0, 1, 2 and 3;
R^{10c} is selected from the group consisting of chloro, fluoro, iodo, and bromo; and
one of R^{40b} and R^{40d} is H and the other of R^{40b} and R^{40d} is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl;
with the proviso that R^{50A} and R^{50B} are not both C₁-C₆ alkyl;
   or a pharmaceutically acceptable salt, solvate, or prodrug thereof.

Some embodiments of the present disclosure are directed to a compound of Formula III:

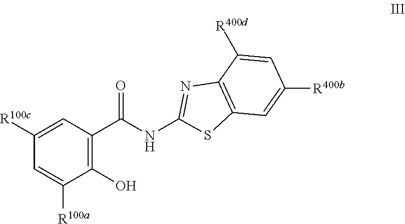

wherein:
R^{100a} is selected from the group consisting of —CH₃, —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)NHCO₂CH₃,

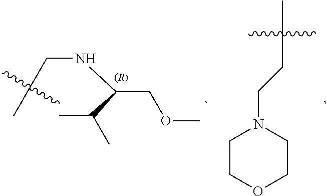

—(CH₂)_{p′}NR^{500A}R^{500B}, —(CH₂)ₚR⁶⁰⁰, and C(O)N(CH₂CH₂OCH₃)₂;
each of R^{500A} and R^{500B} is independently selected from the group consisting of —C₁-C₆ alkyl; and —C₁-C₆ alkyl substituted with one or more groups selected from —C₁-C₆ alkoxy, and —O(CH₂)₂OCH₃; alternatively R^{500A} and R^{500B} together with the nitrogen to which they are attached, form a 4 to 8-membered heterocyclyl optionally substituted with one or more substituents, independently selected from the group consisting of oxo, cyano, hydroxyl, alkoxy, acylamino, carboxyamido, —SO₂CH₃, —CF₃, C₁-C₆ alkyl, halo, and acyl; R⁶⁰⁰ is selected from the group consisting of a 5 to 6-membered heterocyclyl, pyridinyl and thiazolyl;
p′ is an integer selected the group consisting of 1, 2, and 3;
p is an integer selected the group consisting of 0, 1, 2 and 3; R^{100c} is selected from the group consisting of chloro, fluoro, iodo, and bromo;
each of R^{400b} and R^{400d} is independently selected from the group consisting of Y¹ and Z¹, provided that when R^{400b} is Y¹, R^{400d} is Z¹ and when R^{400b} is Z¹, R^{400d} is Y¹;
Y¹ is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl;
Z¹ is selected from the group consisting of —CH₂NHC(O)CH₂CH₃, —CH₂NHC(O)CH₂OCH₃, —CH₂NHSO₂CH₃, —(CH₂)_{q′}NR^{7A}R^{7B}, and —(CH₂)_{q}R⁸;
each of R^{7A} and R^{7B} is independently selected from C₁-C₆ alkyl; alternatively R^{7A} and R^{7B} together with the nitrogen to which they are attached, form a 4 to 8-membered heterocyclyl optionally substituted with one or more independently selected C₁-C₆ alkyl;
R⁸ is selected from the group consisting of a 5 to 6-membered heterocyclic ring optionally substituted with methyl;
q′ is an integer selected from the group consisting of 1, 2, and 3; and
q is an integer selected from the group consisting of 0 1, 2 and 3;
   or a pharmaceutically acceptable salt, solvate, or prodrug thereof.

Embodiments herein describe a pharmaceutical composition comprising: a compound according to any embodiment described herein, or a pharmaceutically acceptable salt or prodrug thereof; and a pharmaceutically acceptable carrier or diluent.

Some embodiments describe a method of treating a mitochondria-related condition or disorder in a subject in need thereof, comprising administering to the subject a therapeutically effective amount of a compound or pharmaceutical composition described herein. In some embodiments the mitochondria-related condition or disorder has and one or more underlying causal factors or symptoms selected from the group consisting of hyperglycemia, abnormal accumulation of lipid in cells, abnormal accumulation of lipid in tissues, abnormal lipid metabolism, insulin resistance, abnormal cell proliferation, abnormal TGF-beta activation, and abnormal fibrosis. In some embodiments, the mitochondria-related condition or disorder is selected from the group consisting of a metabolic disease, cancer, an autoimmune disease, pulmonary fibrosis, a dermatological disorder, an infectious disease, and a neurodegenerative disease.

In some embodiments the metochondria-related condition or disorder is a metabolic disease selected from the group consisting of type 2 diabetes, a disease characterized by insulin resistance or hyperglycemia; obesity or obesity related complications, and a disease characterized by abnormal lipid accumulation. In some embodiments the metabolic disease is a complication caused by type 2 diabetes, selected from the group consisting of diabetes-induced cardiovascular diseases, neurodegenerative disorders, atherosclerosis, hypertension, coronary heart diseases, nephropathy, retinopathy, neuropathy and diabetic heart failure. In some embodiments the metabolic disease or disorder is non-alcoholic fatty liver disease (NAFLD), wherein at least one prognosis stage of the disease is selected from the group consisting of hepatic steatosis, non-alcoholic steatohepatitis (NASH), cirrhosis, and NAFLD induced hepatocellular carcinoma (HCC). In some embodiments the metabolic disease or disorder is alcoholic fatty liver disease, or one or more complications caused by alcoholic fatty liver disease, wherein the one or more complications caused by alcoholic fatty liver disease is selected from the group consisting of alcoholic hepatitis, cirrhosis, and a combination thereof. In some embodiments the metabolic disease or disorder is dyslipidemia, or one or more complications caused by dyslipidemia. In some embodiments the pharmaceutical composition is administered in combination with a second agent indicated for the metabolic disease. In some embodiments the second agent is an anti-diabetic agent selected from the group consisting of metformin, insulin, insulin analogs, sulfonylureas, biguanides, meglit-inides, thiazolidinediones, alpha glucosidase inhibitors, GLP-1 agonists, DPP-4 inhibitors and SGLT2 inhibitors. In some embodiments the second agent s selected from the group consisting of an anti-obesity agent, an anti-nonalcoholic fatty liver disease agent, an anti-nonalcoholic fatty liver disease agent and an anti-dyslipidemia agent.

In some embodiments the metochondria-related condition or disorder is a primary cancer selected from the group consisting of hepatocellular carcinoma, colorectal carcinoma, pancreatic cancer, breast cancer, prostate cancer, leukemia, lymphoma, melanoma, ovarian cancer, and lung cancer. In some embodiments the cancer is a metastatic cancer originated from a primary tumor of other tissue types. In some embodiments the pharmaceutical composition is administered in combination with a second anti-cancer agent or anti-cancer regimen. In some embodiments the second anti-cancer agent is an immunoncological agent. In some embodiments the immunocological agent is selected from the group consisting of an antibody against PD-1/PD-L1, an antibody against other immune check point proteins, CAR-T cells, and other therapeutic immune cells.

In some embodiments the metochondria-related condition or disorder is a dermatological disorder selected from eczema, dyshidrotic eczema, seborrheic eczema psoriasis, rosacea, dermatitis and atopic dermatitis.

In some embodiments the metochondria-related condition or disorder is an infectious disease. In some embodiments the infectious disease is a bacterial infection. In some embodiments the infectious disease is viral infection. In some embodiments the viral infection is selected from SARS-CoV-2 infection, a corona viral infection, and Ebola viral infection.

Some embodiments describe a method of treating a metabolic disease or disorder characterized by hyperglycemia, or insulin resistance or by abnormal accumulation of lipid in tissue, a disease or a disorder in which hyperglycemia, or insulin resistance or abnormal accumulation of lipid in tissue is a symptom, in a subject in need thereof, comprising administering to the subject a therapeutically effective amount of a compound or a pharmaceutical composition described herein.

Some embodiments describe a method of treating cancer or hyperplasia, in a subject in need thereof, comprising administering to the subject a therapeutically effective amount of a compound or a pharmaceutical composition described herein.

Some embodiments describe a method of treating fibrosis, in a subject in need thereof, comprising administering to the subject a therapeutically effective amount of a compound or a pharmaceutical composition described herein.

Some embodiments describe a method of treating or preventing autoimmune diseases in a subject in need thereof, comprising administering to the subject a therapeutically effective amount of oa pharmaceutical composition described herein.

Some embodiments describe a method of treating or preventing a dermatological disorder, in a subject in need thereof, comprising administering to the subject a therapeutically effective amount of a pharmaceutical composition described herein.

Some embodiments describe a method of treating fibrosis in a subject in need thereof, comprising administering to the subject a therapeutically effective amount of a a pharmaceutical composition described herein.

Some embodiments describe a method of treating or preventing a bacterial infection in a subject in need thereof, comprising administering to the subject a therapeutically effective amount of a pharmaceutical composition described herein.

Some embodiments describe a method of treating or preventing viral infection in a subject in need thereof, comprising administering to the subject a therapeutically effective amount of a pharmaceutical composition described herein.

Some embodiments herein describe methods wherein the subject is a mammal or a human. In some embodiments, the subject is a human.

Some embodiments herein describe methods wherein the pharmaceutical composition is administered orally, intravenously, subcutaneously, intramuscularly, transdermally, intraperitoneally, or by other pharmacologically acceptable routes.

Some embodiments describe a method for long-term disease management of a metabolic disease or disorder, or for long-term disease management of cancer, comprising administering to a subject in need of such long-term management an effective amount of a compound or a pharmaceutical composition described herein.

Some embodiments describe the use of a compound described herein in the manufacture of a medicament for treatment of a metabolic disease or disorder. In some embodiments, the metabolic disease or disorder may be selected from diabetes, obesity, non-alcoholic fatty liver disease, alcoholic fatty liver disease, dyslipidemia, a disease where hyperglycemia, or insulin resistance or abnormal lipid accumulation in tissue is a symptom, or related disorders or complications.

Some embodiments describe a method of preparing a mitochondrial membrane potential (MMP)-retaining mitochondrial uncoupler comprising:
1. identifying a conventional mitochondrial uncoupler;
2. designing a compound that covalently links at least one secondary or tertiary amino moiety to a conventional mitochondrial uncoupler; and
3. preparing the compound of step 2, wherein the compound is a mitochondrial membrane retaining uncoupler compound.

Some embodiments describe a mitochondrial membrane-retaining uncoupler compound of the Formula:

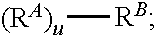

or a pharmaceutically acceptable salt, solvate or prodrug thereof,
wherein R^{A} and R^{B} are covalently linked;
each R^{A} is independently a moiety containing a secondary or tertiary amine;
u is an integer selected from the group consisting of 1 and 2; and
R^{B} is a conventional mitochondrial uncoupler prior to being covalently linked to R^{A}; provided the mitochondrial membrane-retaining uncoupler compound is not

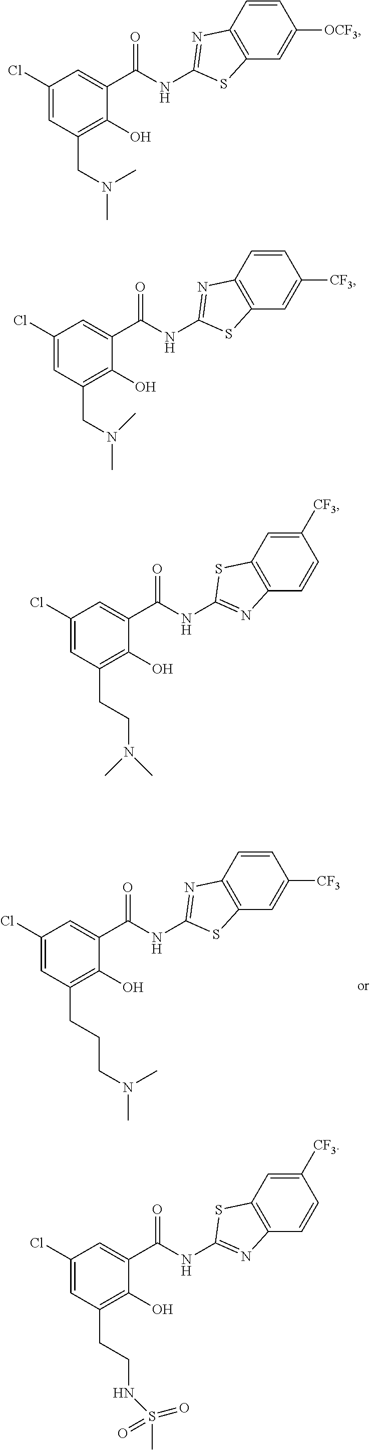 [SMILES: CN(C)Cc1cc(Cl)cc(C(=O)Nc2nc3ccc(OC(F)(F)F)cc3s2)c1O ; CN(C)Cc1cc(Cl)cc(C(=O)Nc2nc3ccc(C(F)(F)F)cc3s2)c1O ; CN(C)CCc1cc(Cl)cc(C(=O)Nc2nc3ccc(C(F)(F)F)cc3s2)c1O ; CN(C)CCCc1cc(Cl)cc(C(=O)Nc2nc3ccc(C(F)(F)F)cc3s2)c1O ; CS(=O)(=O)NCCc1cc(Cl)cc(C(=O)Nc2nc3ccc(C(F)(F)F)cc3s2)c1O]

### DESCRIPTION OF THE DRAWINGS

FIG. 1 shows a schematic diagram illustrating the role of mitochondrial functions and dysfunctions in various diseases, including obesity, T2D (type 2 diabetes), NASH (non-alcoholic steatohepatitis), viral infection, cancer, cancer immune suppression (Cancer ME, cancer microenvironment), and neurodegenerative diseases. Mitochondrial uncoupling, by reducing metabolic output of metabolites and ROS, would help eliminate the causal factors of these diseases.

FIG. 2 shows oxygen consumption rates (OCR) over time of two representative compounds. Compound 16 (FIG. 2A) and Compound 69 (FIG. 2B). Oligo represents oligomycin, Comp. A represents Compound 16, Comp. B represents compound 69, and Rot/AA represents Rotenone/Antimycin. The assay was performed with Seahorse XF-24 instrument. The compounds were injected into assay media in the order as indicated, Oligomycin at 2.5 μM; each of Comp.A and Comp.B at a concentration at 1.0 μM; Rotenone at 2.0 μM and Antimycin A at 2.0 μM.

FIG. 3 shows the molecular mechanism of conventional mitochondrial uncoupling. Dissipating mitochondrial membrane potential is an intrinsic property for conventional mitochondrial uncouplers. The mitochondrial outer membrane, inner membrane, electron transport complexes I-IV, ATP synthase, a chemical uncoupler (U-, UH), and mitochondrial matrix are shown as indicated. Conventional uncouplers are weak lipophilic weak acid localized in mitochondrial inner membrane (shown as the deprotonated form U⁻ and protonated form UH). U⁻ binds to a proton at the outer side of the mitochondrial inner membrane (protonation) and releases the proton into the mitochondrial matrix (deprotonated). As a result, the mitochondrial uncoupler catalyzes proton translocation across the inner membrane without ATP synthesis, leading to ‘futile’ oxidation of acetyl-CoA (an end metabolic product of lipid oxidation and glucose metabolism). As the MMP is established by the proton gradient across the mitochondrial inner membrane, conventional mitochondrial uncoupling is associated with a simultaneous MMP dissipation.

FIG. 4 shows the effect of a a representative conventional mitochondrial uncoupler, trifluoromethoxy carbonylcyanide phenylhydrazone (FCCP), on oxygen consumption rate and mitochondrial membrane potential. FCCP simultaneously increases oxygen consumption rate (FIG. 4A) and dissipates mitochondrial membrane potential (FIG. 4B). FIG. 4A shows cellular oxygen consumption rates (OCR), determined by Seahorse OCR assay using C2C12 cells. The diamond in FIG. 4A represents DMSO (vehicle treatment control), the triangle represents FCCP at 3.0 μM, the square represents FCCP at 6.0 μM, the X represents FCCP at 12.0 μM, the * represents FCCP at 18.0 μM, and the circle represents FCCP at 24 μM. Oligo is oligomycin 2.5 μM; AA isantimycin A 2 μM; Rot isrotenone 2 μM. FIG. 4B shows mitochondrial membrane potential (MMP) determined by fluorescence microscopy using two different dyes, tetramethylrhodamine ethyl ester (TMRE) staining and DilC1(5) staining, respectively, in the presence of FCCP at the indicated concentrations. The fluorescent intensity is indicative of MMP. FCCP has a ratio of C_{10% TMRE}/C_{min-OCR} less than 3, where C_{10% TMRE} is the concentration leading to 10% MMP retention (or 90% MMP loss, measured by TMRE staining) and C_{min-OCR} is the minimal concentration leading to OCR increase.

FIG. 5 shows the effect of a representative MMP-retaining mitochondrial uncoupler, Compound 25 (#25), on oxygen consumption rate and mitochondrial membrane potential. Compound 25 effectively uncouples mitochondria (induces OCR, FIG. 5A) without reducing MMP (FIG. 5B). FIG. 5A shows cellular oxygen consumption rates determined by Seahorse OCR assay using C2C12 cells. The diamond in FIG. 5A represents vehicle treatment control, the square represents Compound 25 (#25) at 1.0 μM, the triangle represents Compound 25 at 3.0 μM, the X represents Compound 25 at 6.0 μM, the * represents Compound 25 at 9.0 μM, and the circle represents Compound 25 at 12.0 μM. Oligo,isoligomycin 2.5 μM; AA is antimycin A 2 μM; Rot isrotenone 2 μM. FIG. 5B shows mitochondrial membrane potential (MMP) determined by fluorescence microscopy using two different dyes, tetramethylrhodamine ethyl ester (TMRE) staining and DilC1(5) staining, respectively, in the presence of Compound 25(#25) at the indicated concentrations. The fluorescent intensity is indicative of MMP. Compound 25 represents the MMP-retaining uncouplers that do not cause observable MMP reduction while uncoupling mitochondria (with a ratio of C_{10% TMRE}/C_{min-OCR} greater than 25).

FIG. 6 shows the effect of Compound 64 (#64), on oxygen consumption rate (FIG. 6A) and mitochondrial membrane potential (FIG. 6B). Compound 64 increases OCR without drastically dissipating mitochondrial membrane potential. FIG. 6A shows cellular oxygen consumption rates determined by Seahorse OCR assay using C2C12 cells at the indicated concentrations. The diamond in FIG. 6A represents vehicle treatment control, the square represents Compound 64 (#64) at 0.3 μM, the triangle represents Compound 64 at 1.0 μM, the X represents Compound 64 at 2.0 μM, the * represents Compound 64 at 3.0 μM, and the circle represents Compound 64 at 4.0 μM. Oligo is oligomycin 2.5 μM; AA is antimycin A 2 μM; Rot is rotenone 2 μM. FIG. 6B shows mitochondrial membrane potential (MMP) determined by fluorescence microscopy using two different dyes, tetramethylrhodamine ethyl ester (TMRE) staining and DilC1(5) staining, respectively, in the presence of Compound 64 (Comp #64) at the indicated concentrations. The fluorescent intensity is indicative of MMP. Compound 64 represents the MMP-retaining uncouplers that do not drastically reduce MMP while uncoupling mitochondria (with a ratio of C_{10% TMRE}/C_{min-OCR} between 10 and 25).

FIG. 7 shows a schematic illustration of the mechanism of action of a conventional mitochondrial uncoupler (FIG. 7A) and a proposed mechanism of action of an MMP-retaining uncoupler (FIG. 7B), as well as diagrams and an example of how to convert a conventional uncoupler to an MMP-retaining uncoupler (FIG. 7C-E). FIG. 7A and FIG. 7B show mitochondrial electron transport chain complexes I, II, III, IV, ATP synthase, as well as uncouplers (UH or U⁻). Conventional uncouplers allow proton translocation across the mitochondrial inner membrane, causing loss of MMP (FIG. 7A). The presence of a positively charged side chain and the asymmetrical distribution of the MMP-retaining uncouplers result in an electrogradient that compensates for the loss of MMP due to proton translocation (FIG. 7B). FIG. 7C and FIG. 7D show a general approach to create new MMP-retaining uncouplers by adding a positively charged side chain to a conventional uncoupler (FIG. 7C), for example, by adding a tertiary amine- (or secondary amine-) containing side chain to obtain an MMP-retaining uncoupler (FIG. 7D), where N is nitrogen; and X and Y are optimally substituted side chains. FIG. 7E shows an example of an MMP-retaining uncoupler, showing a conventional uncoupling component in the frame.

FIG. 8A shows the acute lethal dose leading to the death of 50% of test animals (Acute LD50 or LD50) and the minimum efficacious dose (MED) of the conventional uncoupler DNP (US EPA, 2,4-dinitrophenol. https://www.epa.gov/sites/default/files/2016-09/documents/2-4-dinitrophenol.pdf, FIG. 8A) and MMP-retaining uncouplers, Compound 64 (#64, FIG. 8B) and Compound 25 (#25, FIG. 8C). Compound 64 and Compound 25 exhibit drastically improved acute toxicity profiles over DNP. Acute toxicity tests were performed on C57Bl6 mice, male, 6 weeks old, n=6. Compounds 64 and 25 were prepared to a fine suspension in 0.5% CMC-Na/1% Tween80 water solution. The tests were performed under fed condition, by oral gavage of 100-400 μL compound suspension according to the body weight to desired dosages (mg/kg). The mice were supplied with drinking water all through the testing time. Mouse behaviors were monitored every 15-30 minutes and LD50 were determined. MED was determined in diabetic and steatosis mouse models (Table 9).

FIG. 9 shows the safety profile of Compound 64 (#64) compared to DNP. FIG. 9A shows the NOAEL(no-observable-adverse-effect-level)/MED of DNP. https://www.atsdr.cdc.gov/ToxProfiles/tp64.pdf, (US EPA, 2,4-dinitrophenol. https://www.epa.gov/sites/default/files/2016-09/documents/2-4-dinitrophenol.pdf, and US CDC). FIG. 9B shows the MED, intermediate NOAEL and NOAEL/MED of Compound 64 calculated by oral dosage. FIG. 9C shows the MED, intermediate NOAEL and NOAEL/MED of Compound 64 over DNP calculated by Cmax (maximal blood concentration). FIG. 9D shows the MED, intermediate NOAEL and NOAEL/MED of Compound 64 calculated byAUC (Area Under Curve). The Figures demonstrate that the MMP-retaining uncoupler, Compound 64 exhibits drastically improved short-term safety profiles over the conventional uncoupler DNP. A 10-day toxicology study in CD-1 mice was performed to determine NOAEL of Compound 64, where animals (n=5) were administered once daily for 10 consecutive days. Clinical signs, body weights, food consumptions, rectal temperature, hematology, serum chemistry, plasma exposure, necropsies, tissue histopathology, were examined. MED was determined in diabetic and steatosis mouse models (Table 9).

FIG. 10 show the safety profile of Compound 25 (#25) FIG. 10A shows the MED, intermediate NOAEL and NOAEL/MED of Compound 25 calculated by oral dosage. FIG. 10B shows the MED, intermediate NOAEL and NOAEL/MED of Compound 25 over DNP calculated by Cmax (maximal blood concentration). FIG. 10C shows the MED, intermediate NOAEL and NOAEL/MED of Compound 25 calculated by AUC (Area Under Curve). The Figures demonstrate that the MMP-retaining uncoupler, Compound 25 exhibits drastically improved short-term safety profiles over the conventional uncoupler DNP. A 10-day toxicology study in CD-1 mice was performed to determine NOAEL of Compound 25, where animals (n=5) were administered once daily for 10 consecutive days. Clinical signs, body weights, food consumptions, rectal temperature, hematology, serum chemistry, plasma exposure, necropsies, tissue histopathology, were examined. MED was determined in diabetic and steatosis mouse models (Table 9).

FIG. 11 shows the efficacy of Compound 25 (#25) and Compound 64 (#64) in reducing blood glucose and glycated hemoglobin A1C in the db/db diabetic mouse model. FIG. 11A is a graph of the blood glucose level of mice treated with vehicle and mice treated with Compound 25. FIG. 11B is a graph of glycated hemoglobin A1C of mice treated with vehicle and mice treated with Compound 25. FIG. 11C is a graph of the blood glucose level of mice treated with vehicle and mice treated with Compound 64. FIG. 11D is a graph of glycated hemoglobin A1C of mice treated with vehicle and mice treated with Compound 64. BKS db/db mice were treated with or without 5 mg/kg Compound 25 or Compound64 by daily oral gavage, for 3 weeks. Blood glucose and glycated hemoglobin A1C levels were measured. Statistical significance (P) was determined by the Student's t-test. All error bars, s.d., ***p<0.001. n=6 in each group. Vehicle, vehicle-treated control group.

FIG. 12 shows the effects of Compound 25 on body weight (FIG. 12A), blood glucose (FIG. 12B), liver weight (FIG. 12C), and plasma insulin levels (FIG. D) in HFD-induced diabetic/fatty liver mouse model (n=6 in each group). Statistical significance (P) was determined by the Student's t-test. All error bars, s.d., *p<0.05, ***p<0.001. n=6 in each group. #25: mice treated with Compound 25 (5 mg/kg/day, daily gavage) for three weeks. Vehicle, vehicle-treated control group. FIGS. 12A-B, Light bars, initial levels; dark bars, levels after 3-week vehicle or drug treatment.

FIG. 13 shows the effects of Compound 64 on blood glucose (FIG. 13A) and plasma insulin levels (FIG. 13B) in HFD-induced diabetic/fatty liver mouse model (n=6 in each group). Statistical significance (P) was determined by the Student's t-test. All error bars, s.d., *p<0.05, ***p<0.001. n=6 in each group. #64: mice treated with Compound 64 (5 mg/kg/day, daily gavage) for two weeks. Vehicle, vehicle-treated control group. FIG. 13A, Dark bars, initial levels; light bars, levels after 2-week vehicle or drug treatments.

FIG. 14 shows the effects of Compound 25 on blood triglyceride (FIG. 14A), total cholesterol (FIG. 14B), and non-HDL cholesterol levels (FIG. 14C) in high-fat diet induced diabetic/hepatic steatosis mice. Statistical significance (P) was determined by the Student's t-test. All error bars, s.d., *p<0.05, n=6 in each group. #25: treated with Compound 25 (5 mg/kg/day, daily gavage) for 3 weeks; vehicle, vehicle-treated control group.

FIG. 15 shows the effects of Compound 25 and Compound 64 on hepatic steatosis induced by high-fat diet. Representative liver histology images with H&E staining from mice as indicated: FIG. 15A, normal: the liver section from a healthy C57/Bl6 mouse without HFD feeding; FIG. 15B, HFD, the liver section from a mouse fed HFD; FIG. 15C, HFD+#25, the liver sections from a mouse fed HFD following 3 weeks of Compound 25 treatment (P.O. dosing of 5 mg/kg, by daily gavage); and FIG. 15D, HFD+#64, the liver sections from a mouse fed HFD following 3 weeks Compound 64 treatment (P.O. dosing of 5 mg/kg, by daily gavage). N=6 in each group.

FIG. 16 shows the inhibitory effects of Compound 25 on hepatic stellate cell differentiation into myofibroblast-like cells. FIG. 16A is a schematic representation of the experimental procedure (See procedure B5). FIG. 16B is microscope images showing the cell morphology of LX-2 cells (human hepatic stellate cells) treated with vehicle, 10 ng/mL of TGFβ and TGFβ plus 7.5 ng/mL of Compound 25. TGFβ treatment induces the differentiation of LX-2 cells into myofibroblast-like cells, which form the “ring”-shape morphology after TGFβ treatment (TGFβ, middle panel); Compound 25 (TGFβ+#25, right panel) prevents TGFβ-induced differentiation of LX-2 cells.

FIG. 17 shows the inhibitory effects of Compound 64 on hepatic stellate cell differentiation into myofibroblast-like cells. FIG. 17A. is microscope images showing cell morphology of LX-2 cells (human hepatic stellate cells) treated with vehicle, 10 ng/mL of TGFβ and TGFβ plus 1 ng/mL of Compound 64. TGF-β treatment induces the differentiation of LX-2 cells into myofibroblast-like cells, which form the “ring”-shape morphology after TGF-β treatment (TGF-β, middle panel); Compound64 (TGFβ+#64, right panel) prevents TGFβ-induced differentiation of LX-2 cells. FIG. 17B is an immunoblotting analysis showing Compound 64 blocks activation of TGF-β signaling pathway as evidenced by losing Smad2/3 phosphorylation. LX-2 cells were treated with either vehicle (control), or TGF-β alone, or TGF-β plus indicated concentrations of Compound 64 for 6 hrs. Cells were then harvested, and immunoblotting analyses were performed with antibodies against p-Smad2/3 (phosphorylated Smad2/3), Smad2/3, or GAPDH, as indicated.

FIG. 18 shows the efficacy of Compound 25 in reducing liver fibrosis determined by histology and molecular analysis. FIG. 18A is microscope images of liver sections of CCl₄ treated mice, subjected to H & E staining (top left panel); CCl₄ plus Compound 25 (7.5 mg/kg/day) treated mice subjected to H & E staining (bottom left panel); CCl₄ treated mice, subjected to Picrosirius Red staining(staining fibrotic collagen, top right panel) and CCl₄ plus Compound 25 (7.5 mg/kg/day) treated mice subjected to Picrosirius Red staining (staining fibrotic collagen, bottom left panel. FIG. 18B depicts fibrosis and lipofuscin scoring of vehicle (control), CCl₄ alone, and CCl₄+Compound 25 (CCl₄+25) treated mice. Each slice in a pie represents one mouse. The severity of liver fibrosis and abundance of lipofuscin in each mouse is presented with a different shade of color. Lipofuscin is an intracellular aggregate of highly oxidized proteins and lipids that cannot be digested. Lipofuscin mainly accumulate in lysosomes in aged cells and cells under pathological conditions. FIG. 18C shows the immunoblotting analysis of collagen expression (Col1a1) in mice treated with vehicle, CCl₄ alone, CCl₄+Compound 25 (CCl₄+#25), with the fibrotic scores of each sample listed between the immunoblotting panels, as indicated. GAPDH serves as an internal control. n=7; duration of treatment, 6 weeks. Histological and molecular markers show Compound 25 reduces fibrosis in CCl₄ treated animals.

FIG. 19 is an immunoblotting analysis that shows the inhibitory effect of Compound 6464 on TGF-β activation in T-cells. FIG. 19A is an immunoblotting analysis of Human Jurkat cells treated with either vehicle alone (first lanes), TGF-β alone (second lanes), TGF-β plus Compound 64 at 0.5, 1.0 or 2.0 μM of compound (third through 5th lanes), as indicated, for 6 hours. FIG. 19B is an immunoblotting analysis of mouse primary T-cells (B) were treated with either vehicle alone (first lanes), TGF-β alone (second lanes), or TGF-β plus varying concentrations of Compound 64 at 1.0 or 2.0 μM of compound (third and fourth lanes), as indicated, for 6 hours. Immunoblotting analyses were performed with antibodies against p-Smad2/3 (phosphorylated Smad2/3), Smad2/3, or GAPDH, as indicated. Loss of Smad2/3 phosphorylation indicates the efficacy of Compound 64 in blocking TGF-β activation in T-Cells.

FIG. 20 shows Compound 64 is efficacious in combinatory therapy with PD-1 antibody in treating metastatic cancer in mice. FIG. 20A is the experimental design (see Example B12). Briefly, C57/Bl6 mice intrahepatically transplanted with MC38 cancer cells (day 0) were subject to various treatments starting on day 7: aPD-1 or isotype, mice were either treated with PD-1 antibody or its isotype antibody (control) by intraperitoneal (IP) injection on indicated days (PD-1 antibody has a half-life of over 1 week in mice); #64 or vehicle, mice were either treated with Compound #64 or vehicle by daily gavage. FIG. 20B is a table depicting outcomes of the experiments described in FIG. 20A and Example B12. aPD-1+#64 represents mice treated with PD-1 antibody (IP) and #64 (daily gavage); aPD-1 represents mice treated with PD-1 antibody (IP) and vehicle (gavage); control, represents mice treated with isotype antibody (IP) and vehicle (gavage); n is number of mice in each group. Tumor-positive is the number of tumor-bearing mice in each group; tumor-free is the number of tumor-free mice in each group; tumor-free % is the percentage of tumor-free animals in each group. Fisher Exact 2×2 test is theP value comparing each experimental group to control group using statistical analysis with Fisher Exact 2×2 test. P<0.05 is indicative of a statistically significant difference.

FIG. 21 shows antiviral activity (EC50), cytotoxicity (TC50), and specificity index (SI) of Compounds 64, 25 and 57 against enveloped viruses. Each row represents results from experiments of the indicated compound on the host cells infected with or without the indicated virus. EC50 is the compound concentration that reduces virus-induced cytopathic effects (CPE) by 50%. TC50 is the compound concentration that leads to 50% of cell viability of uninfected cells; SI is the ratio between TC50 and EC50. The experiments were performed as follows. The host cells, either Vero 760 or MRC-5, as indicated, were seeded in 96-well flat-bottom tissue culture plates and allowed to adhere overnight. Following overnight incubation, the cells were either infected with virus (either SARS-CoV-2 or alpha coronavirus 229E) or uninfected, and diluted test compounds were added to each well. Following incubation at 37° C., 5% CO₂ for three days or six days, cell viability was determined. Percent of CPE reduction of the virus-infected wells and the percent of cell viability of uninfected drug control wells, were measured to calculate and determine the EC50 and TC50 values. SI, was calculated accordingly.

### DETAILED DESCRIPTION

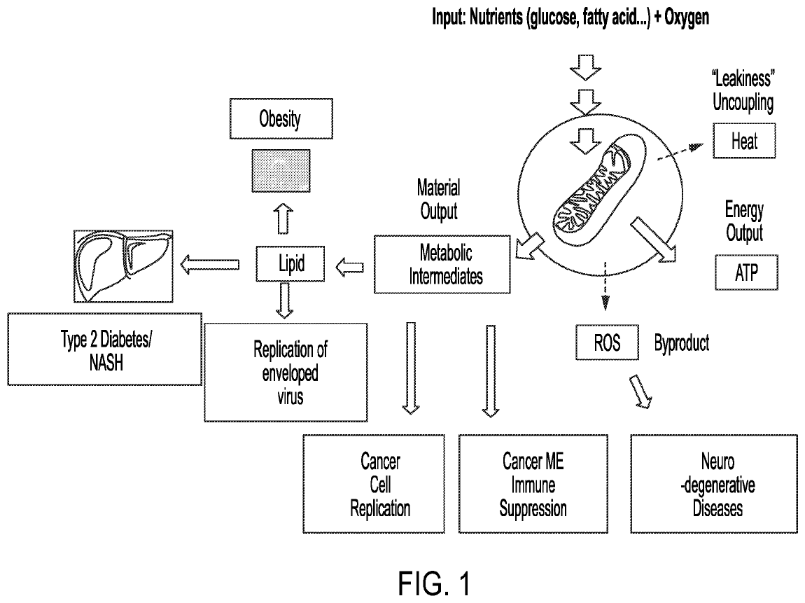

The following factors make mitochondria an ideal target for treating a number of important diseases: (1) mitochondria are the ultimate site where lipid or glucose metabolites are consumed (oxidized); (2) mitochondria are critical in regulating the abundance of metabolic intermediates that become building blocks of biosynthesis essential for cell growth and proliferation of cancer cells, as well as for viral envelop production and assembly; (3) mitochondria are the major production site of ROS in neurons and many other cells (FIG. 1).

Mitochondrial uncoupling is a unique way to modulate mitochondrial activity and functions. In essence, mitochondrial uncoupling is a process by which the activity of mitochondrial electron transport chain is de-coupled from ATP synthesis. Mechanistically, mitochondrial uncoupling is caused by the action of mitochondrial uncouplers that carry protons across the mitochondrial inner membrane into the mitochondrial matrix, independent of the ATP synthase (Terada, H. (1990) Environmental Health Perspectives 87, 213-218). The technical definition of mitochondrial uncouplers is an increase of oxygen consumption rate (OCR) by cells in the presence of an ATP synthase inhibitor such as oligomycin. As a result, mitochondrial uncouplers lead to futile mitochondrial oxidation and increase electron transport chain flux. Consequently, mitochondrial uncouplers could facilitate glucose or lipid catabolism in cells, reduce small molecule building block production output, and reduce electron stall in electron transport chain, hence reduce electron leakiness and mitochondrial ROS production. Through these actions, mitochondrial uncouplers represent an effective strategy for potential treatment of a number of important diseases.

Mitochondria and metabolic diseases. Metabolic diseases are a family of diseases characterized by symptoms of abnormal glucose and/or lipid metabolism, such as obesity, type 2 diabetes, alcoholic fatty liver disease, non-alcoholic fatty liver diseases, non-alcoholic steatohepatitis. These diseases are associated with age-, environmental-, or genetic-related decrease in mitochondrial functions such as reduced oxidative capacity. Importantly, these diseases also share a common causal factor, namely abnormal accumulation of intracellular lipid in cells of various tissues as well as insulin resistance in most cases. For example, obesity is characterized by excessive fat accumulation in cells of adipose tissue. Metabolic syndrome is characterized with insulin resistance in peripheral tissues, usually caused by ectopic fat accumulation in cells of liver, muscle, or adipose tissue. Type 2 diabetes is characterized by insulin resistance usually caused by ectopic fat accumulation in cells of liver, muscle, or adipose tissue, and hyperglycemia caused by insulin resistance. Alcoholic fatty liver disease is characterized by ectopic lipid accumulation in liver cells and liver damage, liver inflammation, and fibrosis. The various stages of non-alcoholic liver fatty liver disease (or NAFLD), include hepatosteatosis, non-alcoholic steatohepatitis (NASH), cirrhosis, and NAFLD induced hepatocellular carcinoma (HCC), has a primary cause of ectopic lipid accumulation in liver cells which lead to liver damage, inflammation, and fibrosis. The various types of dyslipidemia are partly caused by ectopic accumulation of lipid in cells of liver, muscle, or heart, as a result of redistribution of lipid from adipose tissue to other tissues.

Mitochondrial uncouplers, which reduce energy efficiency and boost futile lipid oxidation, would effectively reduce cellular accumulation of lipid. As ectopic intracellular accumulation of lipid in the liver and muscle, as well as excessive accumulation of lipid in adipose tissue are the fundamental cause of insulin resistance in various forms of metabolic diseases (Samuel V. T., et al., Lancet, 2010, 375:2267-77), applicant and others have demonstrated in animal models, that small molecule (chemical) mitochondrial uncouplers are efficacious in preventing and treating metabolic diseases (Tao, H., Zhang, Y., Zeng, X., Shulman, G. I., and Jin, S. (2014) Nature Medicine, 20, 1263-1269; Perry, R. J., Zhang, D., Zhang, X. M., Boyer, J. L., and Shulman, G. I. (2015) Science, 347(6227), 1253-6), leading to: (1) reduction of lipid accumulation in various tissues, including adipose tissue, (2) reduction in insulin resistance, (3) reduction in blood glucose concentrations, and (4) improvement in glycemic control and slowdown in disease progression. Importantly, using mitochondrial uncouplers for treating metabolic diseases has a number of appealing features; for example, since they correct the cause of insulin resistance (ectopic lipid accumulation), such an approach may provide a cure for some metabolic diseases.

Cancer is a family of diseases characterized by uncontrolled growth and proliferation of cells of various tissue types, resulting from a combination of genetic mutations in oncogenes and tumor suppressor genes. It is well-accepted that one requirement of tumorigenesis is the alteration of cell metabolism. Cancer cells require not only energy but also the building blocks (metabolic intermediates) for biosynthesis of macromolecules such as DNA and RNA to support rapid cell growth and proliferation. Metabolism in cancer cells is changed in such a way it could support both the energy need and the need of the various metabolic intermediates (building blocks) for biosynthesis of macromolecules (Vander Heiden, M. G., Cantley, L. C., and Thompson, C. B. (2009) Science, 324(5930), 1029-33). As a result, most cancers exhibit a unique cellular metabolic pattern called the Warburg effect, or aerobic glycolysis, which prevents the complete oxidation of glucose or lipid and allows for production of glucose metabolites for biosynthesis of macromolecules (Vander Heiden, et al., 2009).

Mitochondrial uncoupling reduces energy efficiency thereby undermining the energy requirement of cancer cells. In addition, mitochondrial uncoupling promotes the complete mitochondrial oxidation of glucose and lipid, thereby diminishing the production of metabolic intermediates essential for biosynthesis of macromolecules required for cell proliferation. Moreover, mitochondrial uncoupling could lead to AMPK activation, a known event for inhibiting cell growth. Indeed, prior documents showed that mitochondrial uncouplers exhibit anti-cancer activities (U.S. Pat. No. 10,227,315). Targeting cancer cells through mitochondrial uncoupling would deprive energy as well as biosynthetic metabolic intermediates that are absolutely essential for cancer cell growth and proliferation, which is proven to be an effective anti-cancer strategy (Alasadi, A. et al., (2018) Cell Death Dis., 9(2), 215)

Autoimmune diseases are conditions where the body's immune system attacks their own healthy organs. The common autoimmune diseases include celiac disease, diabetes mellitus type 1, Graves' disease, inflammatory bowel disease, multiple sclerosis, psoriasis, rheumatoid arthritis, and systemic lupus erythematosus. To activate and sustain the autoimmune activity, the body's own self-attaching immune cells need amplification (proliferation) which requires metabolic changes similar to the Warburg effect observed in cancer cells to provide sufficient building blocks for biosynthesis (Ganeshan, K., et al. (2014) Annual Review of Immunology, 32, 609-634). Therefore, mitochondrial uncoupling would potentially inhibit the activation and amplification of self-attacking immune cells.

Neurodegenerative diseases are a large group of disabling disorders of the nervous system such as Parkinson's disease, Huntington's disease, amyotrophic lateral sclerosis, and Alzheimer's disease, characterized by the relative selective death of neuronal subtypes. There is overwhelming evidence of impaired mitochondrial function as a causative factor in these diseases, with mitochondrial ROS production as one of the most important one (Elfawy, H. A., and Das, B. (2019) Life Sci., 218, 165-184.)

Mitochondrial uncouplers increase mitochondrial electron transport chain flux and decrease the electron stall in the complexes of the electron transport chain, thus they could effectively reduce mitochondrial ROS. Therefore, mitochondrial uncoupling is considered a powerful anti-oxidant strategy and could have therapeutic potential for treating neurodegenerative diseases.

A decline in mitochondrial function and capacity has been associated with normal aging and correlated with the development of a wide range of age-related diseases. Some proven ant-aging and pro-longevity approaches, such as calorie restriction, highly correlate with improvement in mitochondrial functions (Sun, N., et al. (2016) Mol. Cell, 61(5), 654-666). Modulation of mitochondrial functions by mitochondrial uncoupling has been proposed as anti-aging strategy (Caldeira da Silva, C. C., et al. (2008) Aging Cell, 7(4), 552-60).

Mitochondria are ancient bacteria that formed a symbiotic relationship with host cells. Bacterial plasma membrane contains electron transport chain and ATP synthase that are similar to those of mitochondria, therefore compounds that impact mitochondrial uncoupling could be useful inhibitors of bacterial growth and effective as antibiotics (U.S. Pat. No. 10,227,315).

Despite the appealing beneficial features of mitochondrial uncoupling, no U. S. FDA approved drug is currently being used in humans with the mechanism of action of mitochondrial uncoupling for treating the above mentioned diseases in United States. There are major hurdles for developing mitochondrial uncouplers as therapeutics. Discovery of new synthetic mitochondrial uncouplers with better druggable features is critical for developing mitochondrial uncoupling therapeutics.

Benzamide mitochondrial uncouplers have been developed (International Patent Publication Number WO 2012/068274, International Patent Publication Number WO 2016/081599, U.S. Pat. No. 10,227,315, and Tao et al., 2014) for potential therapeutic applications. One main limitation of the prior benzamide compounds are poor pharmacokinetic properties and low systemic exposure. For example, in order to overcome the poor pharmacokinetic properties of low systemic exposure and short half-life of the previously disclosed compounds, studies in animal models required the compound to be mixed with food, and high doses of the compounds were required to achieve efficacy (e.g. 1500 ppm niclosamide ethanolamine (NEN) in diet, equivalent to 150 mg/kg/day (Tao, et al. 2014, and WO 2012/068274); or 600-750 ppm of Compound 27 (in U.S. Pat. No. 10,227,315) in diet, equivalent of 60-75 mg/kg/day). Neither the high doses required nor the need to mix the compound in food, is compatible with therapeutic development in humans.

Another limitation of the available mitochondrial uncouplers is safety concerns, because some mitochondrial uncouplers are known to have a narrow therapeutic index. For example, the most famous mitochondrial uncoupler, 2,4-dinitrophenol, was previously used in humans but was withdrawn from the market due to its narrow therapeutic window. 2,4-Dinitrophenol has a ratio of concentration (in cells)/dosage (in vivo), between toxicity and efficacy, is merely a factor of about 3. Therefore, new properties that allow mitochondrial uncouplers that improve the safety margin are considered critical for therapeutic development of mitochondrial uncouplers.

Various embodiments provide new benzamide mitochondrial uncouplers that effectively induce mitochondrial uncoupling (increase mitochondrial oxygen consumption in the presence of oligomycin) without significantly decreasing mitochondrial membrane potential (vide infra) over wide concentration ranges. These compounds are referred to herein as mitochondrial membrane potential-retaining (MMP-retaining) compounds (MMP-retaining).

These MMP-retaining compounds exhibit drastically improved safety profiles compared to the bench mark conventional mitochondrial uncoupler, DNP. These compounds exhibit a wider therapeutic index when used for treating metabolic diseases.

Various embodiments provide new benzamide mitochondrial uncouplers that show markedly improved pharmacokinetic properties, such as markedly increased systemic exposure (with some compounds having more than 100 fold increase in AUC than some previously disclosed druggable benzamide mitochondrial uncouplers with the highest reported systemic exposure).

Various embodiments describe the use of these compounds for the prevention and treatment of bacterial infections; dermatological disorders; viral infection; metabolic diseases or disorders, including, but not limited to, obesity, metabolic syndrome, type 2 diabetes, alcoholic fatty liver disease, non-alcoholic fatty liver diseases, dyslipidemia, and primary cancer of various tissue origins, and metastatic cancer.

Various embodiments provide the use of these compounds for treatment of disease symptoms such as hyperglycemia, insulin resistance, aberrant lipid accumulation, fibrosis, and aberrant TGF-β activation.

Various embodiments describe the use of these compounds for the prevention and treatment of the diseases alone or in combination with another agent.

### Compounds

In one embodiment, the present disclosure describes a compound of Formula A:

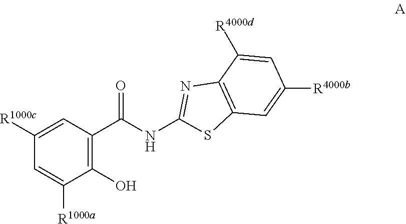

or a pharmaceutically acceptable salt, solvate, or prodrug thereof.

The substituent R^{1000a} of Formula A is selected from the group consisting of

—CH₃, —CH₂CH₃, —C₁-C₆ alkyl, —C₃-C₆ cycloalkyl, —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)NHCO₂CH₃,

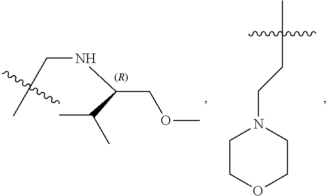

—(CH₂)_{r′}NR^{5000A}R^{5000B}, —(CH₂)ᵣR⁶⁰⁰⁰, and C(O)N(CH₂CH₂OCH₃)₂. Each of substituents R^{5000A} and R^{5000B}, is independently selected from the group consisting of —C₁-C₆ alkyl; and —C₁-C₆ alkyl substituted with one or more groups selected from —C₁-C₆ alkoxy, and —O(CH₂)₂OCH₃; with the proviso that R^{5000A} and R^{5000B} are not both C₁-C₆ alkyl. Alternatively R^{5000A} and R^{5000B} together with the nitrogen to which they are attached, form a 4 to 8-membered heterocyclyl optionally substituted with one or more substituents, independently selected from the group consisting of oxo, cyano, hydroxyl, alkoxy, acylamino, carboxyamido, —SO₂CH₃, —CF₃, C₁-C₆ alkyl, halo, and acyl. Integer r′ is an integer selected the group consisting of 1, 2 and 3. Substituent R⁶⁰⁰⁰ is selected from the group consisting of a 5 to 6-membered heterocyclyl, pyridinyl and thiazolyl; and r is an integer selected from the group consisting of 0, 1, 2, and 3.

Substituent R^{1000c} of Formula I is selected from the group consisting of chloro, fluoro, iodo, and bromo.

Each of substituents R^{4000b} and R^{4000d} of Formula A is independently selected from the group consisting of Y¹⁰⁰⁰ and Z¹⁰⁰⁰, provided that when R^{4000b} is Y, R^{4000d} is Z and when R^{4000b} is Z, R^{4000d} is Y. Substituent Y¹⁰⁰⁰ is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl. Substituent Z¹⁰⁰⁰ is selected from the group consisting of H,

—CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃, —(CH₂)O(CH₂)₂NR^{2000A}R^{2000B}, —(CH₂)ₛR³⁰⁰⁰, —CH₂OCH₂Ar¹, —OCH₃CH₂NHC(O)CH₂CH₃, —CH₂NHC(O)CH₂OCH₃, —CH₂NHSO₂CH₃, —(CH₂)_{t′}NR^{7000A}R^{7000B}, and —(CH₂)ₜR⁸⁰⁰⁰ provided that when Z¹⁰⁰⁰ is H; R^{1000a} is not C₁-C₆ alkyl, —C₃-C₆ cycloalkyl, —CH₃ or —CH₂CH₃. Substituents R^{2000A} and R^{2000B} together with the nitrogen to which they are attached, form a 4 to 8-membered heterocyclic ring optionally substituted with one or more methyl groups. Substituent R³⁰⁰⁰ is a 5 to 6-membered heterocyclic ring and s is an integer selected from the group consisting of 0, 1, 2 and 3. Substituent Ar^{t} is a 5-6 membered aryl or heteroaryl group optionally substituted with one or more substituents, independently selected from C₁-C₆ alkyl, halo, hydroxyl, and alkoxy. Each of R^{7000A} and R^{7000B} is independently selected from
C₁-C₆ alkyl. Alternatively R^{7000A} and R^{7000B} together with the nitrogen to which they are attached, form a 4 to 8-membered heterocyclyl optionally substituted with one or more substituents independently selected from C₁-C₆ alkyl and t′ is an integer selected from the group consisting of 1, 2, and 3. Substituent R⁸⁰⁰⁰ is selected from the group consisting of a 5 to 6-membered heterocyclic ring optionally substituted with methyl, and t is an integer selected from the group consisting of 0, 1, 2 and 3.

In some embodiments R^{4000d} is selected from the group consisting of H, R^{4d} as described in paragraph [0078] and R^{400d} as described in paragraph [0149].

In some embodiments the compound of Formula A is a compound selected from the group consisting of a compound of Formula I, a compound of Formula II and a compound of Formula III as defined herein.

In some embodiments a compound of Formula A is selected from the group consisting of a compound as described in any of paragraphs [0089], [0120] and [0156].

In one embodiment, the present disclosure describes a compound of Formula I:

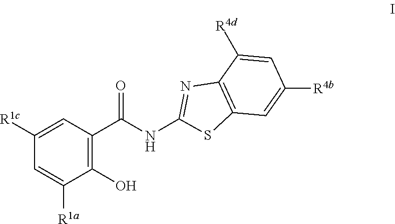

or a pharmaceutically acceptable salt, solvate, or prodrug thereof.

The substituent R^{1a} of Formula I is selected from the group consisting of —CH₃, —CH₂CH₃, —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)NHCO₂CH₃,

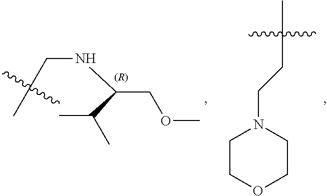

—(CH₂)_{m′}NR^{5A}R^{5B}, —(CH₂)ₘR⁶, and C(O)N(CH₂CH₂OCH₃)₂. Each of substituents R^{5A} and R^{5B}, is independently selected from the group consisting of —C₁-C₆ alkyl; and —C₁-C₆ alkyl substituted with one or more groups selected from —C₁-C₆ alkoxy, and —O(CH₂)₂OCH₃. Alternatively R^{5A} and R^{5B} together with the nitrogen to which they are attached, form a 4 to 8-membered heterocyclyl optionally substituted with one or more substituents, independently selected from the group consisting of oxo, cyano, hydroxyl, alkoxy, acylamino, carboxyamido, —SO₂CH₃,
—CF₃, C₁-C₆ alkyl, halo, and acyl. Integer m′ is an integer selected the group consisting of 1, 2 and 3. Substituent R⁶ is selected from the group consisting of a 5 to 6-membered heterocyclyl, pyridinyl and thiazolyl; and m is an integer selected from the group consisting of 0, 1, 2 and 3.

Substituent R^{1c} of Formula I is selected from the group consisting of chloro, fluoro, iodo, and bromo.

Each of substituents R^{4b} and R^{4d} of Formula I is independently selected from the group consisting of Y and Z, provided that when R^{4b} is Y, R^{4d} is Z and when R^{4b} is Z, R^{4d} is Y. Substituent Y is selected from the group consisting of chloro, fluoro, iodo, bromo, —CF₃,

—CHF₂, fluoro(C₁-C₆)alkyl, halo(C₁-C₆)alkyl, —OCF₃, —SO₂(C₁-C₆)alkyl, cyano, and —CO₂(C₁-C₆)alkyl. Substituent Z is selected from the group consisting of —CH₂OH, —CH₂OCH₂CH₃, —CH₂OCH₃, —CH₂O(CH₂)₂OH, —CH₂O(CH₂)₂OCH₃, —CH₂O(CH₂)₂NHCH₃, —CH₂O(CH₂)₂N(CH₃)₂, —CH₂O(CH₂)₂NHSO₂CH₃, —(CH₂)O(CH₂)₂NR^{2A}R^{2B}, —(CH₂)ₙR³, —CH₂OCH₂Ar, and OCH₃. Substituents R^{2A} and R^{2B} together with the nitrogen to which they are attached, form a 4 to 8-membered heterocyclic ring optionally substituted with one or more methyl groups. Substituent R³ is selected from the group consisting of a 5 to 6-membered heterocyclic ring and phenoxy; and n is an integer selected from the group consisting of 0 1, 2 and 3. Substituent Ar is a 5-6 membered aryl or heteroaryl group optionally substituted with one or more substituents, independently selected from C₁-C₆ alkyl, halo, hydroxy, and alkoxy.

Further embodiments describe the compound according to Formula Ia:

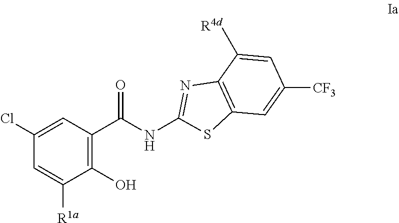

or a pharmaceutically acceptable salt, solvate, or prodrug thereof, wherein each of R^{1a} and R^{4d} of Formula Ia is as described previously for Formula I.

In some embodiments, R^{1a} of Formula I, or of Formula Ia or a pharmaceutically acceptable salt, solvate, or prodrug thereof, is selected from —CH₂NR^{5A}R^{5B}; wherein each of R^{5A} and R^{5B} is independently selected from methyl; and C₁-C₆ alkyl substituted with one or more groups selected from methoxy, and —O(CH₂)₂OCH₃.

In some embodiments, R^{1a} of Formula I, or of Formula Ia or a pharmaceutically acceptable salt, solvate, or prodrug thereof, is selected from the group consisting of —CH₂NR^{5A}R^{5B}; wherein each of R^{5A} and R^{5B} is independently selected from C₁-C₆ alkyl substituted with one or more methoxy.

In some embodiments, R^{1a} of Formula I, or of Formula Ia or a pharmaceutically acceptable salt, solvate, or prodrug thereof, is selected from the group consisting of —CH₂NR^{5A}R^{5B}; wherein R^{5A} and R^{5B} together with the nitrogen to which they are attached, form a 4 to 8-membered heterocyclyl optionally substituted with one or more substituents, independently selected from the group consisting of oxo, cyano, hydroxyl, alkoxy,

-acylamino, carboxyamido, —SO₂CH₃, —CF₃, C₁-C₆ alkyl, halo, and acyl. In some embodiments the 4-8-membered heterocyclyl is selected from the group consisting of azetindinyl, pyrrolidinyl, piperidinyl, piperazinyl, oxopiperazinyl, morpholinyl, dioxothiomorphylinyl, and azabicyclo[3.2.1]octanyl; wherein the heterocyclyl is optionally substituted with one or more substituents, independently selected from the group consisting of oxo, cyano, hydroxyl, alkoxy,
-acylamino, carboxyamido, —SO₂CH₃, —CF₃, C₁-C₆ alkyl, halo, and acyl. In some embodiments the 4-8-membered heterocyclyl is optionally substituted with one or more substituents independently selected from the group consisting of oxo, cyano, hydroxyl, methoxy,
—NHC(O)CH₃, —C(O)NH₂, —SO₂CH₃, —CF₃, methyl, fluoro, and acetyl. In some embodiments the 4-8-membered heterocyclyl is optionally substituted with one or more methyl substituents. In some embodiments the 4 to 8-membered heterocyclyl is selected from the group consisting of azetindinyl; pyrrolidinyl; pyrrolidinyl substituted with one or more substituents selected from the group consisting of cyano, hydroxyl, methoxy, —NHC(O)CH₃, —C(O)NH₂, —SO₂CH₃, —CF₃ and methyl; piperidinyl; piperidinyl substituted with one or more substituents selected from the group consisting of —CF₃, and fluoro; piperazinyl substituted with one or more substituents selected from the group consisting of oxo, methyl, and acetyl; morpholinyl; morpholinyl substituted with one or more methyl groups;
dioxothiomorphylinyl, and azabicyclo[3.2.1]octanyl.

In some embodiments, R^{1a} of Formula I, or of Formula Ia or a pharmaceutically acceptable salt, solvate, or prodrug thereof, is selected from the group consisting of —CH₂NR^{5A}R^{5B}; wherein R^{5A} and R^{5B} together with the nitrogen to which they are attached, form a 6-membered heterocyclyl optionally substituted with one or more substituents, independently selected from the group consisting of oxo, cyano, hydroxyl, alkoxy, -acylamino, carboxyamido, —SO₂CH₃, —CF₃, C₁-C₆ alkyl, halo, and acyl. In some embodiments the 6-membered heterocyclyl is selected from the group consisting piperidinyl optionally substituted with one or C₁-C₆ alkyl substituents, and morpholinyl optionally substituted with one or C₁-C₆ alkyl substituents.

In some embodiments, R^{1a} of Formula I, or of Formula Ia or a pharmaceutically acceptable salt, solvate, or prodrug thereof, is selected from the group consisting of —CH₂NR^{5A}R^{5B}; wherein —CH₂NR^{5A}R^{5B} is selected from the group consisting of —CH₂N(CH₃)(CH₂)₂OCH₃, —(CH₂)N(CH₂CH₂OCH₃)₂, —CH₂N(CH₃)(CH₂)₂O(CH₂)₂OCH₃,

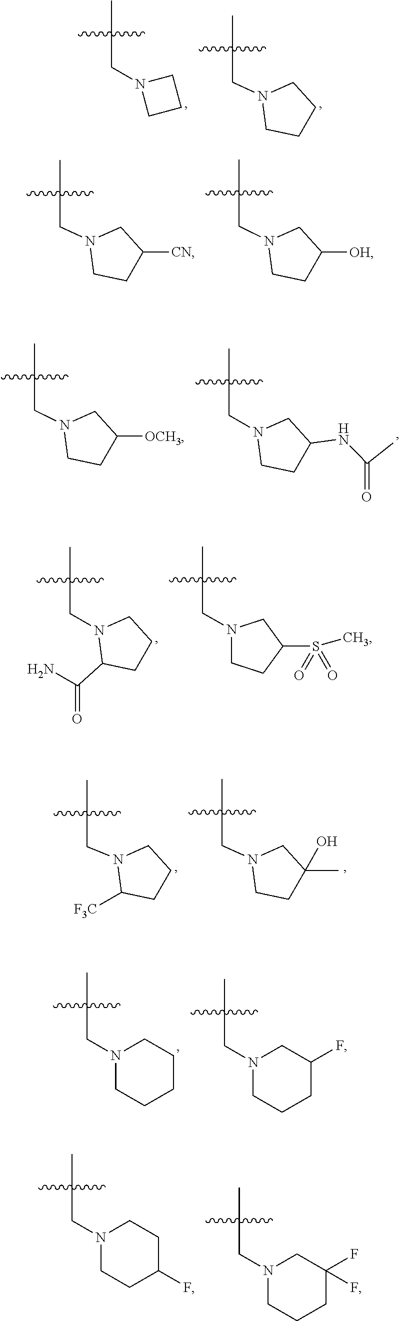

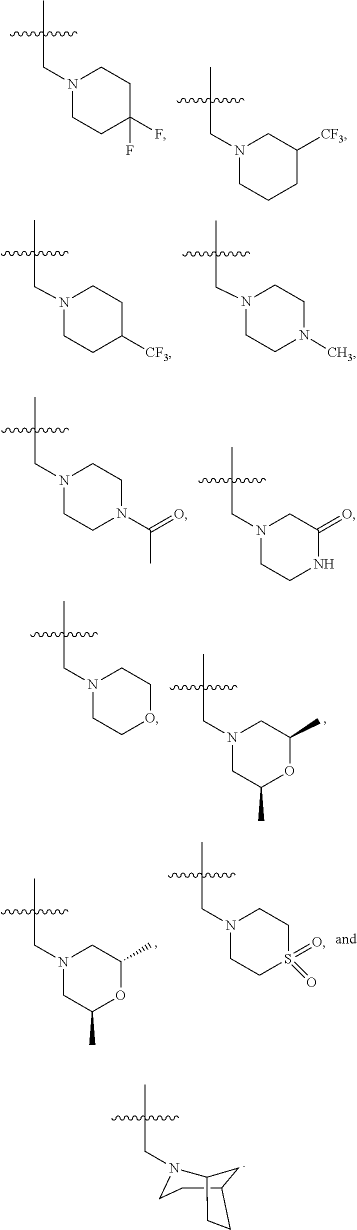

In some embodiments —CH₂NR^{5A}R^{5B} is selected from the group consisting of —(CH₂)N(CH₂CH₂OCH₃)₂

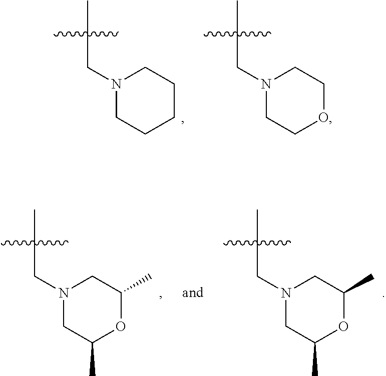

In some embodiments, R^{1a} of Formula I, or of Formula Ia or a pharmaceutically acceptable salt, solvate, or prodrug thereof, is —(CH₂)ₘR⁶; wherein R⁶ is selected from the group consisting of tetrahydrofuranyl, tetrahydropyranyl, piperidinyl, thiazolyl and piperidinyl and m is as previously described. In some embodiments m is 0. In some embodiments m is 1. In some embodiments (CH₂)ₘR⁶ is selected from the group consisting of

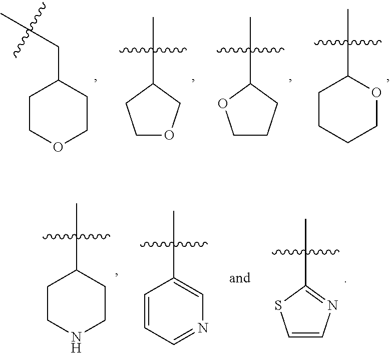

In some embodiments, R^{1a} of Formula I, or of Formula Ia or a pharmaceutically acceptable salt, solvate, or prodrug thereof, is selected from the group consisting of —CH₃, —CH₂CH₃, —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)₂NHCO₂CH₃,

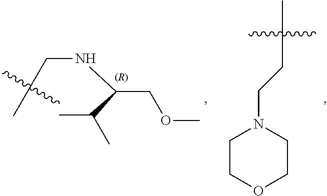

—CH₂N(CH₃)(CH₂)₂OCH₃, —(CH₂)N(CH₂CH₂OCH₃)₂, —CH₂N(CH₃)(CH₂)₂O(CH₂)₂OCH₃,

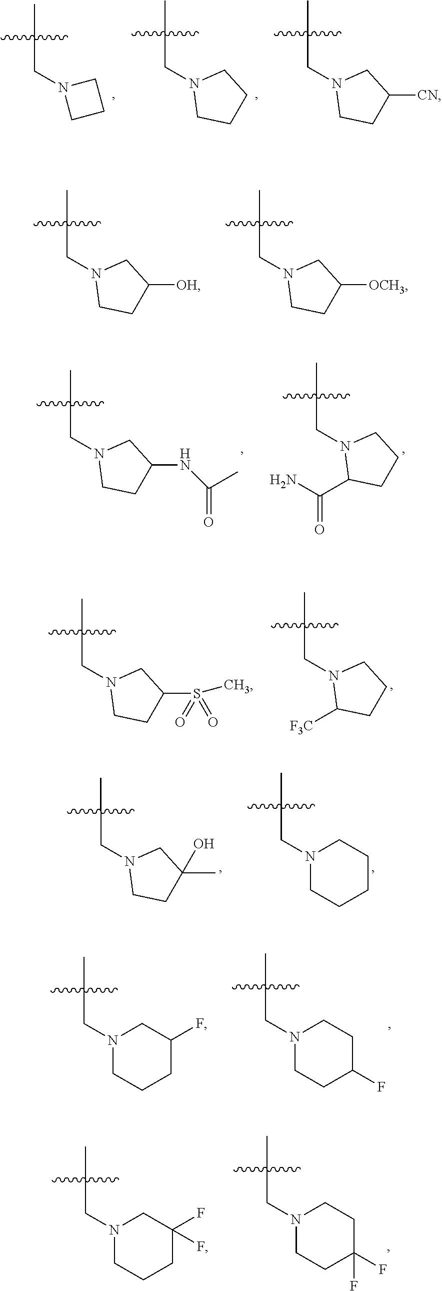

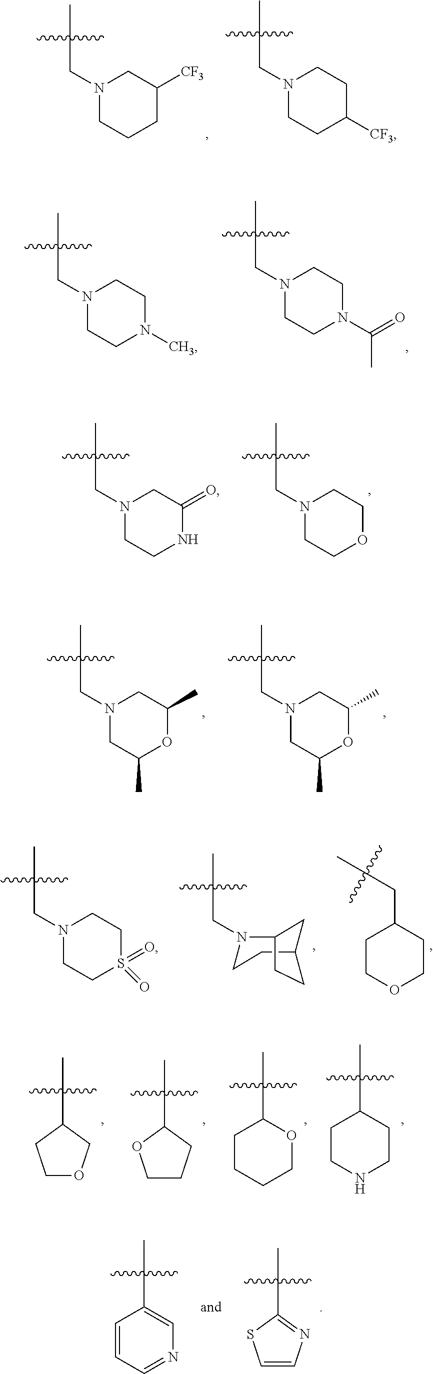

In some embodiments, R^{1a} of Formula I, or of Formula Ia or a pharmaceutically acceptable salt, solvate, or prodrug thereof, is selected from the group consisting of —CH₃, —CH₂CH₃ and —CH₂NR^{5A}R^{5B} wherein R^{5A} and R^{5B} are as previously described in any embodiment described herein. In some embodiments, each of R^{5A} and R^{5B} is independently selected from C₁-C₆ alkyl substituted with one or more methoxy; alternatively, R^{5A} and R^{5B} together with the nitrogen to which they are attached, form a 6-membered heterocyclyl optionally substituted with one or more substituents, independently selected from C₁-C₆ alkyl.

In some embodiments, R^{1a} of Formula I, or of Formula Ia or a pharmaceutically acceptable salt, solvate, or prodrug thereof, is selected from the group consisting of —CH₃, —CH₂CH₃, —(CH₂)N(CH₂CH₂OCH₃)₂,

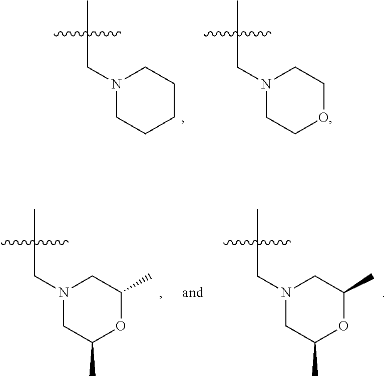

In some embodiments, R^{1c} of Formula I, or a pharmaceutically acceptable salt, solvate, or prodrug thereof, is chloro.

Some embodiments describe a compound of Formula I, or a pharmaceutically acceptable salt, solvate, or prodrug thereof, wherein R^{4b} is Y and R^{4d} is Z.

Some embodiments describe a compound of Formula I, or a pharmaceutically acceptable salt, solvate, or prodrug thereof, wherein R^{4d} is Y and R^{4b} is Z.

In some embodiments, Y of Formula I, or a pharmaceutically acceptable salt, solvate, or prodrug thereof, is —CF₃.

In some embodiments, Z of Formula I, or R^{4d} of Formula Ia, or a pharmaceutically acceptable salt, solvate, or prodrug thereof, is —(CH₂)O(CH₂)₂NR^{2A}R^{2B} wherein R^{2A} and R^{2B} are as previously described herein. In some embodiments R^{2A} and R^{2B} together with the nitrogen to which they are attached, form a 6-membered heterocyclyl optionally substituted with a methyl group. In some embodiments R^{2A} and R^{2B} together with the nitrogen to which they are attached, form a heterocyclyl selected from piperazinyl or a 4-methyl piperazinyl.

In some embodiments, Z of Formula I, or R^{4d} of Formula Ia, or a pharmaceutically acceptable salt, solvate, or prodrug thereof, is —(CH₂)ₙR³, wherein R³ is as previously described herein. In some embodiments R³ is a 5-membered heterocyclic ring. In some embodiments R³ is selected from the group consisting of tetrahydrofuranyl, and phenoxy. In some embodiments R³ is tetrahydrofuranyl. In some embodiments R³ is selected from the group consisting of

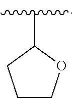 [SMILES: *C1CCCO1]

and phenoxy. In some embodiments n is 0. In some embodiments n is 1. In some embodiments —(CH₂)ₙR³ is selected from the group consisting of

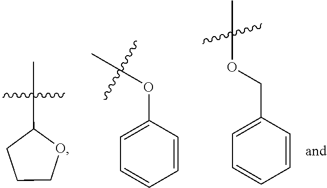 [SMILES: *C1CCCO1 ; *Oc1ccccc1 ; *OCc1ccccc1]

In some embodiments —(CH₂)ₙR³ is

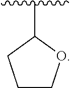 [SMILES: *C1CCCO1]

In some embodiments, Z of Formula I, or R^{4d} of Formula Ia, or a pharmaceutically acceptable salt, solvate, or prodrug thereof, is —CH₂OCH₂Ar, wherein Ar is as previously described herein. In some embodiments Ar is a 5 to 6-membered aryl or heteroaryl optionally substituted with one or more substituents independently selected from the group consisting of methyl, fluoro, chloro, hydroxy, and methoxy. In some embodiments Ar is selected from the group consisting of thiazolyl, phenyl, and phenyl substituted with one or more groups independently selected from methyl, fluoro, chloro, hydroxy, and methoxy.

In some embodiments Ar is selected from the group consisting of, phenyl,

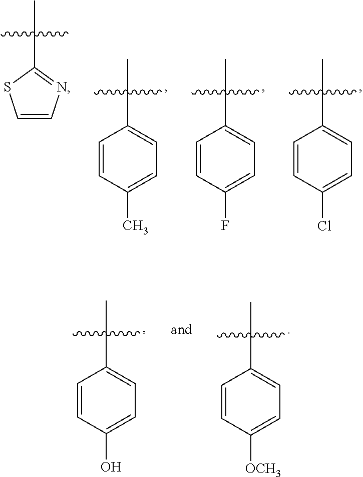

In some embodiments, Z of Formula I, or R^{4d} of Formula Ia, or a pharmaceutically acceptable salt, solvate, or prodrug thereof, is selected from the group consisting of —CH₂OH, —CH₂OCH₂CH₃, —CH₂OCH₃, —CH₂O(CH₂)₂OH, —CH₂O(CH₂)₂OCH₃, CH₂O(CH₂)₂NHCH₃, —CH₂O(CH₂)₂N(CH₃)₂, —CH₂O(CH₂)₂NHSO₂CH₃, —(CH₂)O(CH₂)₂NR^{2A}R^{2B}, —(CH₂)ₙR³, —CH₂OCH₂Ar and OCH₃; wherein R^{2A}R^{2B}, R³, n and Ar are as described in any embodiment herein.

In some embodiments, Z of Formula I, or R^{4d} of Formula Ia, or a pharmaceutically acceptable salt, solvate, or prodrug thereof, is selected from the group consisting of —CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃, —(CH₂)O(CH₂)₂NR^{2A}R^{2B}, —(CH₂)ₙR³, —CH₂OCH₂Ar and OCH₃; wherein R^{2A}R^{2B}, R³, n and Ar are as described in any embodiment herein.

In some embodiments, Z of Formula I, or R^{4d} of Formula Ia, or a pharmaceutically acceptable salt, solvate, or prodrug thereof, is selected from the group consisting of —CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃, —(CH₂)O(CH₂)₂NR^{2A}R^{2B}, —(CH₂)ₙR³, —CH₂OCH₂Ar and OCH₃; wherein R^{2A} and R^{2B} together with the nitrogen to which they are attached, form a heterocyclyl selected from the group consisting of piperazinyl and 4-methyl piperazinyl; R³ is tetrahydrofuranyl; and Ar is a 5 to 6-membered aryl or heteroaryl group optionally substituted with one or more substituents independently selected from the group consisting of methyl, fluoro, chloro, hydroxy, and methoxy.

In some embodiments Z of Formula I, or R^{4d} of Formula Ia, or a pharmaceutically acceptable salt, solvate, or prodrug thereof, is selected from the group consisting of —CH₂OH, —CH₂OCH₂CH₃, —CH₂OCH₃, —CH₂O(CH₂)₂OH, —CH₂O(CH₂)₂OCH₃, —CH₂O(CH₂)₂NHCH₃, —CH₂O(CH₂)₂N(CH₃)₂, —CH₂O(CH₂)₂NHSO₂CH₃,

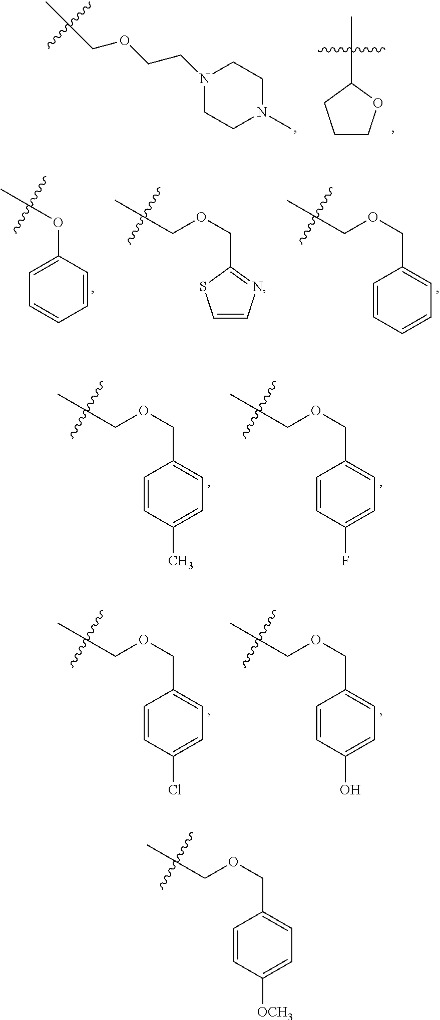

In some embodiments Z of Formula I, or R^{4d} of Formula Ia, or a pharmaceutically acceptable salt, solvate, or prodrug thereof, is selected from the group consisting of —CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃,

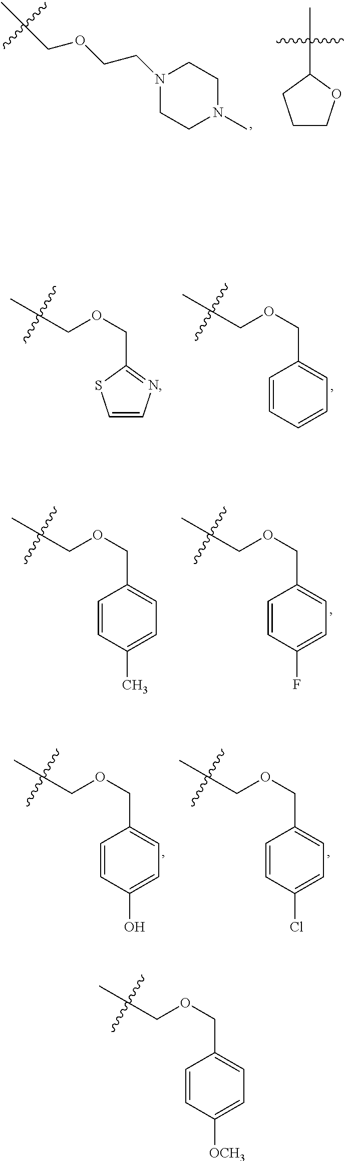

In some embodiments Z of Formula I or R^{4d} of Formula Ia, or a pharmaceutically acceptable salt, solvate, or prodrug thereof, is selected from the group consisting of —CH₂OH, —CH₂OCH₂CH₃, —CH₂OCH₃, —CH₂O(CH₂)₂OH, —CH₂O(CH₂)₂OCH₃, —CH₂O(CH₂)₂NHCH₃, —CH₂O(CH₂)₂N(CH₃)₂, —CH₂O(CH₂)₂NHSO₂CH₃,

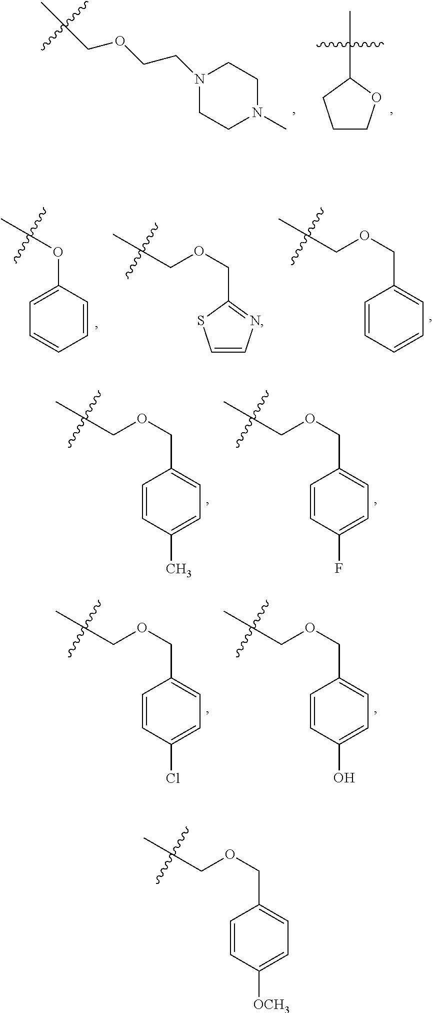

and —OCH₃ and Y of Formula I is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl.

In some embodiments Z of Formula I or R^{4d} of Formula Ia, or a pharmaceutically acceptable salt, solvate, or prodrug thereof, is selected from the group consisting of —CH₂OH, —CH₂OCH₂CH₃, —CH₂OCH₃, —CH₂O(CH₂)₂OH, —CH₂O(CH₂)₂OCH₃, —CH₂O(CH₂)₂NHCH₃, —CH₂O(CH₂)₂N(CH₃)₂, —CH₂O(CH₂)₂NHSO₂CH₃,

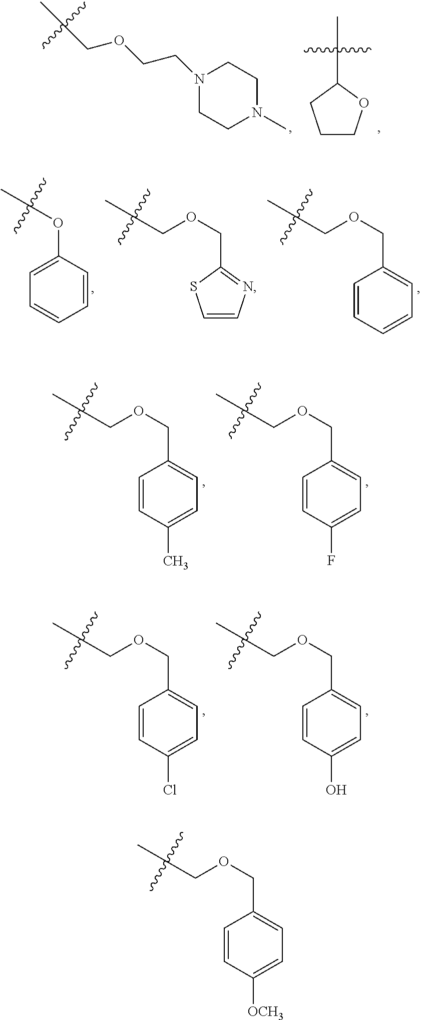

and —OCH₃ and Y is —CF₃.

In some embodiments Z of Formula I or R^{4d} of Formula Ia, or a pharmaceutically acceptable salt, solvate, or prodrug thereof, is selected from the group consisting —CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃,

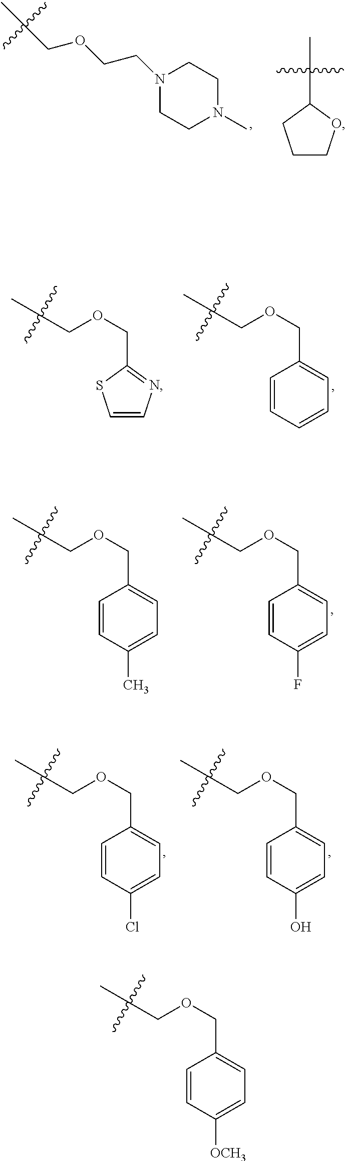

—OCH₃ and Y is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl.

In some embodiments Z of Formula I or R^{4d} of Formula Ia, or a pharmaceutically acceptable salt, solvate, or prodrug thereof, is selected from the group consisting —CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃,

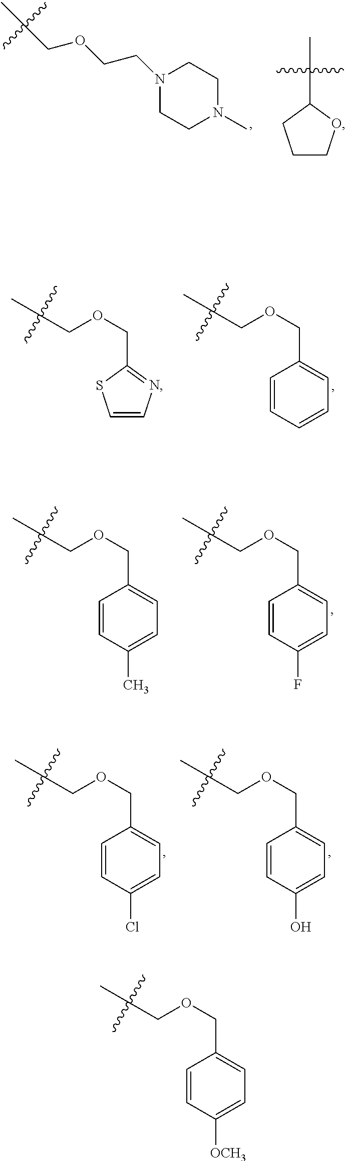

and —OCH₃ and Y is —CF₃.

Some embodiments describe a compound of Formula I wherein:
R^{1a} is selected from the group consisting of —CH₃, —CH₂CH₃ and —CH₂NR^{5A}R^{5B};
each of R^{5A} and R^{5B} is independently selected from C₁-C₆ alkyl substituted with one or more groups selected from methoxy; alternatively, R^{5A} and R^{5B} together with the nitrogen to which they are attached, form a 4-8-membered heterocyclyl optionally substituted with one or more substituents, independently selected from C₁-C₆ alkyl;
R^{1c} is chloro;
each of R^{4b} and R^{4d} is independently selected from the group consisting of Y and Z, provided that when R^{4b} is Y, R^{4d} is Z and when R^{4b} is Z, R^{4d} is Y;
Y is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl;
Z is selected from the group consisting of —CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃, —(CH₂)O(CH₂)₂NR^{2A}R^{2B}, —(CH₂)ₙR³, —CH₂OCH₂Ar and —OCH₃; wherein R^{2A} and R^{2B} together with the nitrogen to which they are attached, form a heterocyclyl selected from the group consisting of piperazinyl and 4-methyl piperazinyl; R³ is tetrahydrofuranyl; and Ar is a 5 to 6-membered aryl or heteroaryl group optionally substituted with one or more substituents independently selected from the group consisting of methyl, fluoro, chloro, hydroxy, and methoxy.

Some embodiments describe a compound of Formula I or a pharmaceutically acceptable salt, solvate, or prodrug thereof, wherein:
R^{1a} is selected from the group consisting of —CH₃, —CH₂CH₃ and —CH₂NR^{5A}R^{5B}; wherein each of R^{5A} and R^{5B} is independently selected from C₁-C₆ alkyl substituted with one or more groups selected from methoxy; alternatively, R^{5A} and R^{5B} together with the nitrogen to which they are attached, form a 6-membered heterocyclyl optionally substituted with one or more substituents, independently selected from C₁-C₆ alkyl;
R^{1c} is chloro;
each of R^{4b} and R^{4d} is independently selected from the group consisting of Y and Z, provided that when R^{4b} is Y, R^{4d} is Z and when R^{4b} is Z, R^{4d} is Y; Y is CF₃; and Z is selected from the group consisting of —CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃, —(CH₂)O(CH₂)₂NR^{2A}R^{2B}, —(CH₂)ₙR³,
   —CH₂OCH₂Ar and —OCH₃; wherein R^{2A} and R^{2B} together with the nitrogen to which they are attached, form a heterocyclyl selected from the group consisting of piperazinyl and 4-methyl piperazinyl; R³ is tetrahydrofuranyl; and Ar is a 5 to 6-membered aryl or heteroaryl group optionally substituted with one or more substituents independently selected from the group consisting of methyl, fluoro, chloro, hydroxy, and methoxy.

Some embodiments describe a compound of Formula I or a pharmaceutically acceptable salt, solvate, or prodrug thereof, wherein:
R^{1a} is selected from the group consisting of —CH₃, —CH₂CH₃, —(CH₂)N(CH₂CH₂OCH₃)₂,

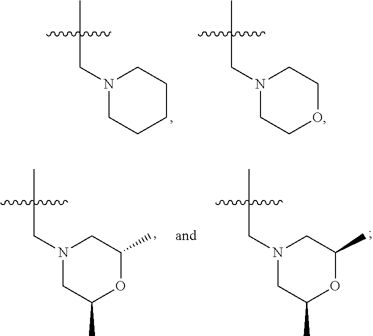

R^{1c} is chloro;
each of R^{4b} and R^{4d} is independently selected from the group consisting of Y and Z, provided that when R^{4b} is Y, R^{4d} is Z and when R^{4b} is Z, R^{4d} is Y; Y is CF₃; and Z is selected from the group consisting of —CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃,

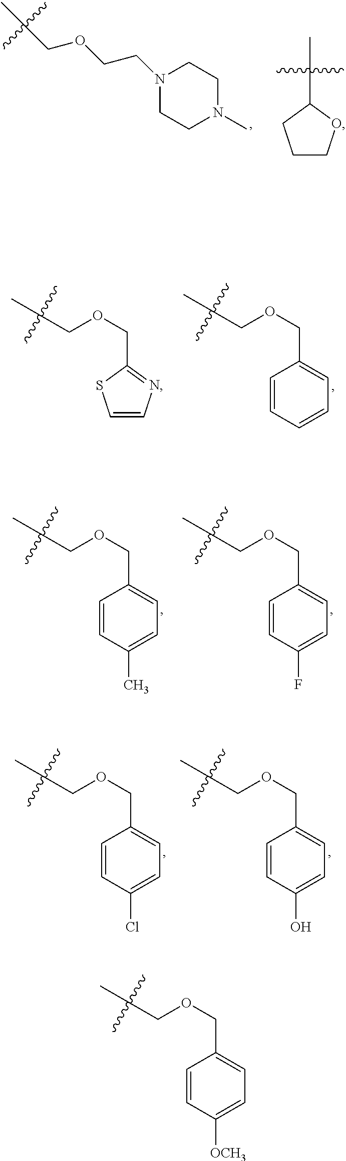

### and —OCH₃.

Some embodiments describe a compound of Formula I or a pharmaceutically acceptable salt, solvate, or prodrug thereof, wherein:
R^{1a} is selected from the group consisting of —CH₃, —CH₂CH₃, —(CH₂)N(CH₂CH₂OCH₃)₂

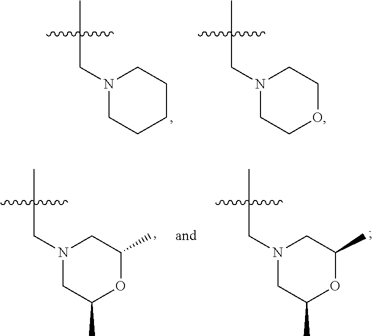

R^{1c} is chloro;
R^{4b} is CF₃; and
R^{4d} is selected from the group consisting of —CH₂OCH₂CH₃, —CH₂OCH₃, —CH₂O(CH₂)₂OH, —CH₂O(CH₂)₂OCH₃, —CH₂O(CH₂)₂N(CH₃)₂, —CH₂O(CH₂)₂NHSO₂CH₃,

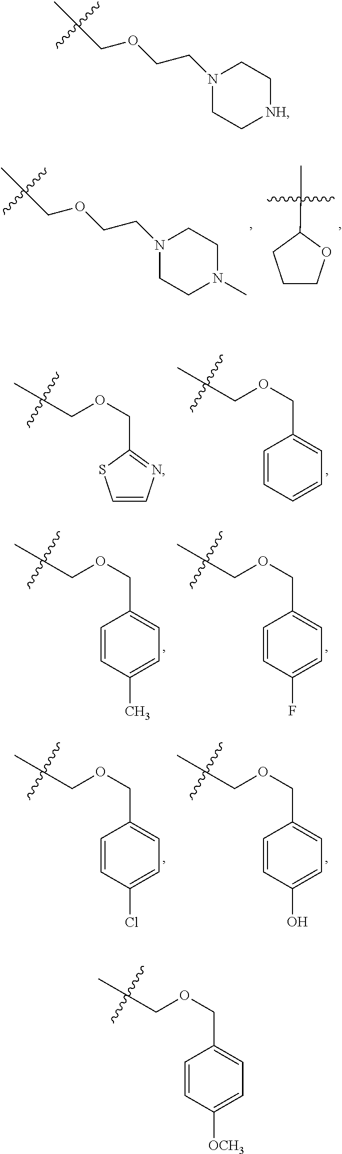

### and —OCH₃.

Some embodiments describe a compound of Formula Ia or a pharmaceutically acceptable salt, solvate, or prodrug thereof, wherein R^{1a} is selected from the group consisting of —CH₃, —CH₂CH₃, —(CH₂)N(CH₂CH₂OCH₃)₂,

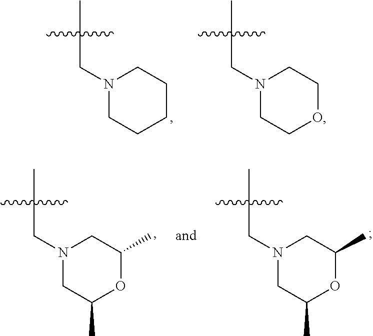

and R^{4d} is selected from the group consisting of CH₂OH, —CH₂OCH₂CH₃, —CH₂OCH₃, —CH₂O(CH₂)₂OH, —CH₂O(CH₂)₂OCH₃, —CH₂O(CH₂)₂NHCH₃, —CH₂O(CH₂)₂N(CH₃)₂, —CH₂O(CH₂)₂NHSO₂CH₃,

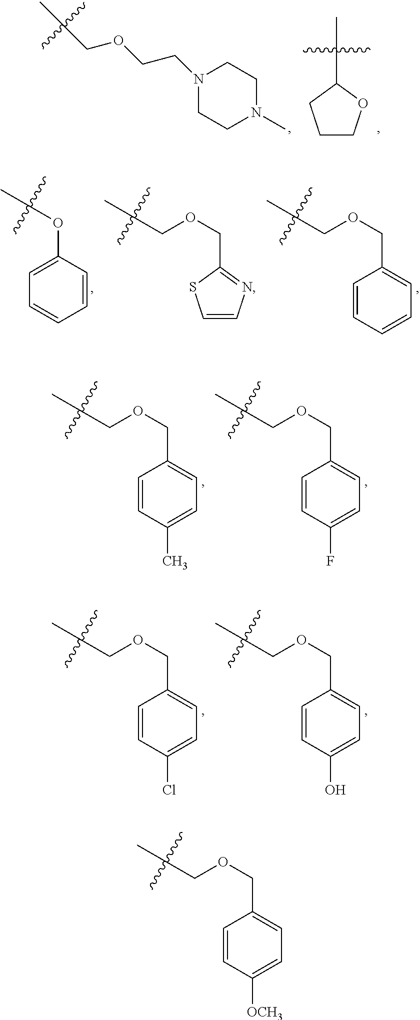

### and —OCH₃.

Some embodiments describe a compound of Formula Ia or a pharmaceutically acceptable salt, solvate, or prodrug thereof, wherein R^{1a} is selected from the group consisting of

—CH₃, —CH₂CH₃, —(CH₂)N(CH₂CH₂OCH₃)₂,

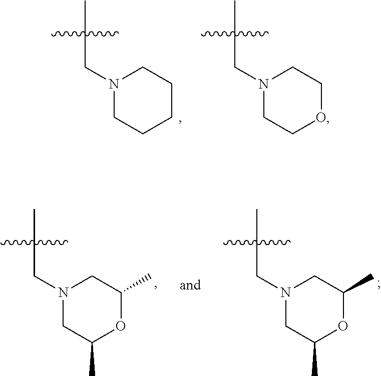

and R^{4d} is selected from the group consisting of —CH₂OCH₃, —CH₂O(CH₂)₂OH, —CH₂O(CH₂)₂OCH₃, —CH₂O(CH₂)₂NHCH₃, —CH₂O(CH₂)₂N(CH₃)₂, —CH₂O(CH₂)₂NHSO₂CH₃,

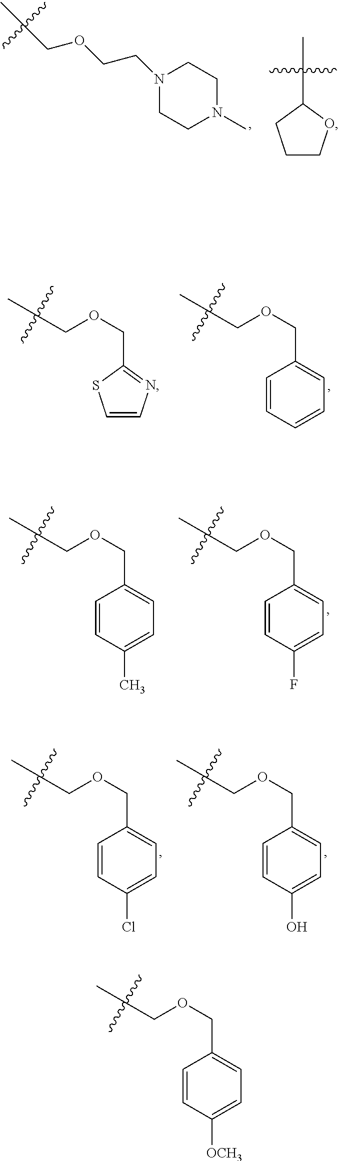

### and —OCH₃.

Some embodiments describe a compound selected from the group consisting of:

| Number | Structure | Name |
|---|---|---|
| 1001 | 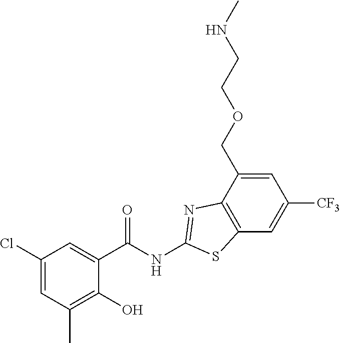 [SMILES: CNCCOCc1cc(C(F)(F)F)cc2sc(NC(=O)c3cc(Cl)cc(C)c3O)nc12] | 5-chloro-2-hydroxy-3-methyl-N-(4-((2- (methylamino)ethoxy)methyl)-6- (trifluoromethyl)benzo[d]thiazol-2- yl)benzamide |
| 1002 | 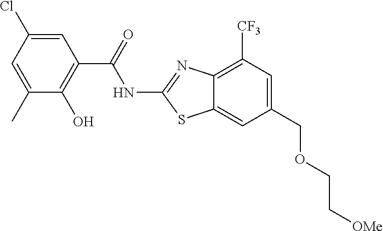 [SMILES: COCCOCc1cc(C(F)(F)F)c2nc(NC(=O)c3cc(Cl)cc(C)c3O)sc2c1] | 5-chloro-2-hydroxy-N-(6-((2- methoxyethoxy)methyl)-4- (trifluoromethyl)benzo[d]thiazol-2-yl)-3- methylbenzamide |
| 1003 | 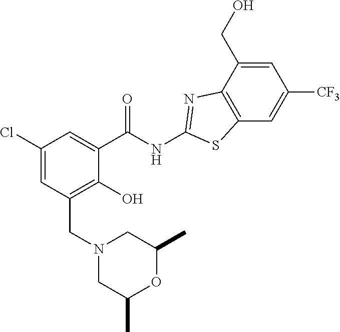 [SMILES: C[C@@H]1CN(Cc2cc(Cl)cc(C(=O)Nc3nc4c(CO)cc(C(F)(F)F)cc4s3)c2O)C[C@H](C)O1] | 5-chloro-3-((cis-2,6- dimethylmorpholino)methyl)-2-hydroxy-N-(4- (hydroxymethyl)-6- (trifluoromethyl)benzo[d]thiazol-2- yl)benzamide |
| 1004 | 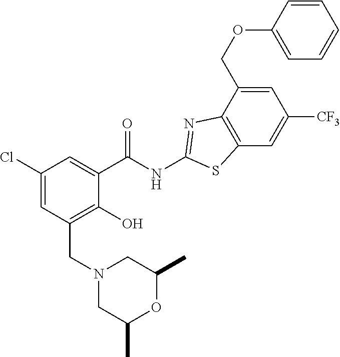 [SMILES: C[C@@H]1CN(Cc2cc(Cl)cc(C(=O)Nc3nc4c(COc5ccccc5)cc(C(F)(F)F)cc4s3)c2O)C[C@H](C)O1] | 5-chloro-3-((cis-2,6- dimethylmorpholino)methyl)-2-hydroxy-N-(4- (phenoxymethyl)-6- (trifluoromethyl)benzo[d]thiazol-2- yl)benzamide |
| 1005 | 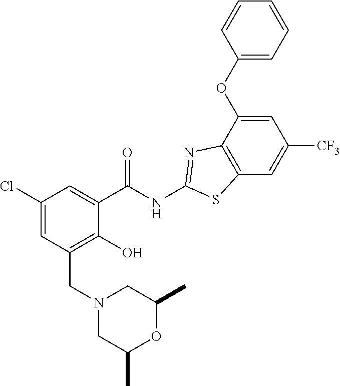 [SMILES: C[C@@H]1CN(Cc2cc(Cl)cc(C(=O)Nc3nc4c(Oc5ccccc5)cc(C(F)(F)F)cc4s3)c2O)C[C@H](C)O1] | 5-chloro-3-((cis-2,6- dimethylmorpholino)methyl)-2-hydroxy-N-(4- phenoxy-6-(trifluoromethyl)benzo[d]thiazol-2- yl)benzamide |
| 1006 | 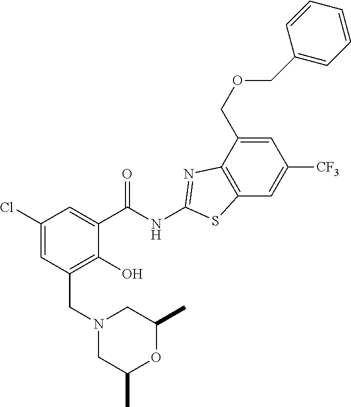 [SMILES: C[C@@H]1CN(Cc2cc(Cl)cc(C(=O)Nc3nc4c(COCc5ccccc5)cc(C(F)(F)F)cc4s3)c2O)C[C@H](C)O1] | N-(4-((benzyloxy)methyl)-6- (trifluoromethyl)benzo[d]thiazol-2-yl)-5- chloro-3-((cis-2,6- dimethylmorpholino)methyl)-2- hydroxybenzamide |
| 13 | 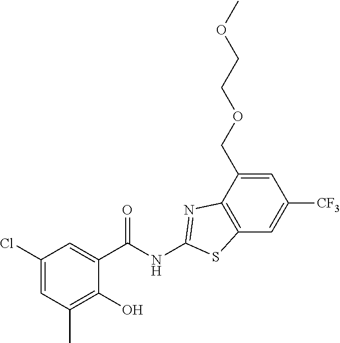 [SMILES: COCCOCc1cc(C(F)(F)F)cc2sc(NC(=O)c3cc(Cl)cc(C)c3O)nc12] | 5-chloro-2-hydroxy-N-(4-((2- methoxyethoxy)methyl)-6- (trifluoromethyl)benzo[d]thiazol-2-yl)-3- methylbenzamide |
| 63 | 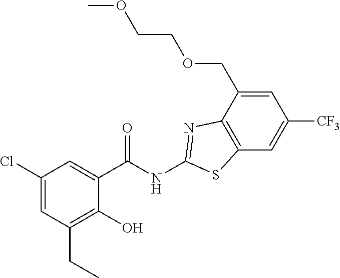 [SMILES: CCc1cc(Cl)cc(C(=O)Nc2nc3c(COCCOC)cc(C(F)(F)F)cc3s2)c1O] | 5-chloro-2-hydroxy-N-(4-((2- methoxyethoxy)methyl)-6- (trifluoromethyl)benzo[d]thiazol-2-yl)-3- methylbenzamide |
| 64 | 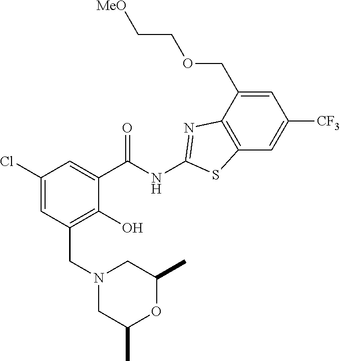 [SMILES: COCCOCc1cc(C(F)(F)F)cc2sc(NC(=O)c3cc(Cl)cc(CN4C[C@@H](C)O[C@@H](C)C4)c3O)nc12] | 5-chloro-3-((cis-2,6- dimethylmorpholino)methyl)-2-hydroxy-N-(4- ((2-methoxyethoxy)methyl)-6- (trifluoromethyl)benzo[d]thiazol-2- yl)benzamide (64) 5-chloro-3-((cis-2,6- dimethylmorpholino)methyl)-2-hydroxy-N-(4- ((2-methoxyethoxy)methyl)-6- (trifluoromethyl)benzo[d]thiazol-2- yl)benzamide hydrochloride (64A) |
| 65 | 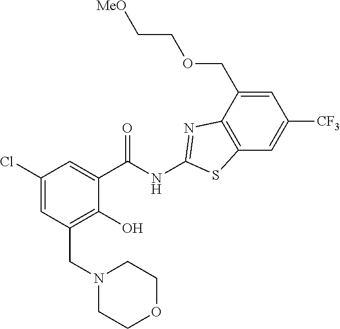 [SMILES: COCCOCc1cc(C(F)(F)F)cc2sc(NC(=O)c3cc(Cl)cc(CN4CCOCC4)c3O)nc12] | 5-chloro-2-hydroxy-N-(4-((2- methoxyethoxy)methyl)-6- (trifluoromethyl)benzo[d]thiazol-2-yl)-3- (morpholinomethyl)benzamide |

| | | |
|---|---|---|
| 66 | 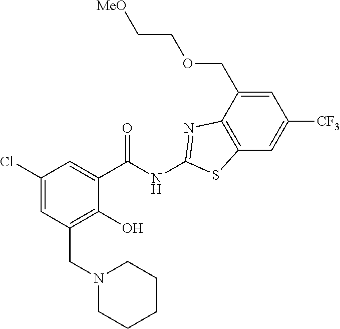 [SMILES: COCCOCc1cc(C(F)(F)F)cc2sc(NC(=O)c3cc(Cl)cc(CN4CCCCC4)c3O)nc12] | 5-chloro-2-hydroxy-N-(4-((2- methoxyethoxy)methyl)-6- (trifluoromethyl)benzo[d]thiazol-2-yl)-3- (piperidin-1-ylmethyl)benzamide |

| | | |
|---|---|---|
| 67 | 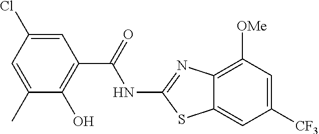 [SMILES: COc1cc(C(F)(F)F)cc2sc(NC(=O)c3cc(Cl)cc(C)c3O)nc12] | 5-chloro-2-hydroxy-N-(4-methoxy-6- (trifluoromethyl)benzo[d]thiazol-2-yl)-3- methylbenzamide |

| | | |
|---|---|---|
| 68 | 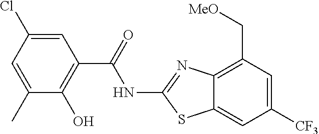 [SMILES: COCc1cc(C(F)(F)F)cc2sc(NC(=O)c3cc(Cl)cc(C)c3O)nc12] | 5-chloro-2-hydroxy-N-(4-(methoxymethyl)-6- (trifluoromethyl)benzo[d]thiazol-2-yl)-3- methylbenzamide |

| | | |
|---|---|---|
| 69 | 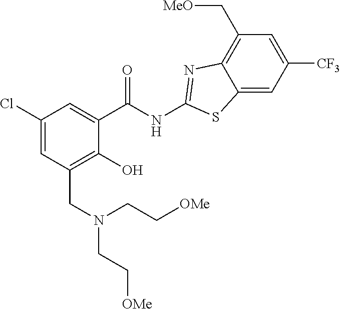 [SMILES: COCCN(CCOC)Cc1cc(Cl)cc(C(=O)Nc2nc3c(COC)cc(C(F)(F)F)cc3s2)c1O] | 3-((bis(2-methoxyethyl)amino)methyl)-5- chloro-2-hydroxy-N-(4-(methoxymethyl)-6- (trifluoromethyl)benzo[d]thiazol-2- yl)benzamide |

| | | |
|---|---|---|
| 70 | 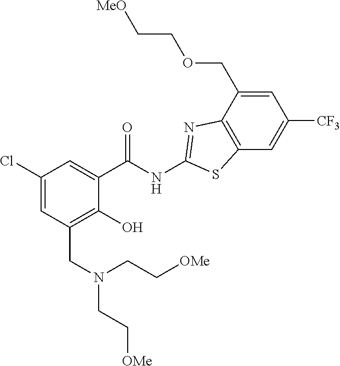 [SMILES: COCCOCc1cc(C(F)(F)F)cc2sc(NC(=O)c3cc(Cl)cc(CN(CCOC)CCOC)c3O)nc12] | 3-((bis(2-methoxyethyl)amino)methyl)-5- chloro-2-hydroxy-N-(4-((2- methoxyethoxy)methyl)-6- (trifluoromethyl)benzo[d]thiazol-2- yl)benzamide |

| | | |
|---|---|---|
| 71 | 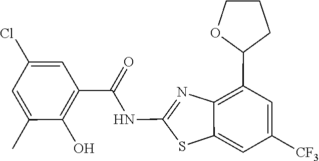 [SMILES: Cc1cc(Cl)cc(C(=O)Nc2nc3c(C4CCCO4)cc(C(F)(F)F)cc3s2)c1O] | 5-chloro-2-hydroxy-3-methyl-N-(4- (tetrahydrofuran-2-yl)-6- (trifluoromethyl)benzo[d]thiazol-2- yl)benzamide |

| | | |
|---|---|---|
| 75 | 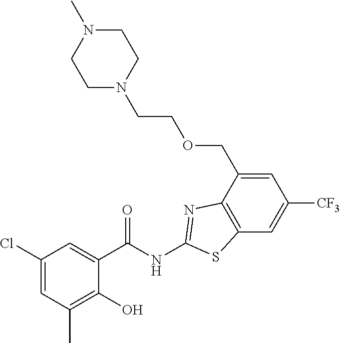 [SMILES: Cc1cc(Cl)cc(C(=O)Nc2nc3c(COCCN4CCN(C)CC4)cc(C(F)(F)F)cc3s2)c1O] | 5-chloro-2-hydroxy-3-methyl-N-(4-((2-(4- methylpiperazin-1-yl)ethoxy)methyl)-6- (trifluoromethyl)benzo[d]thiazol-2- yl)benzamide |

| | | |
|---|---|---|
| 76 | 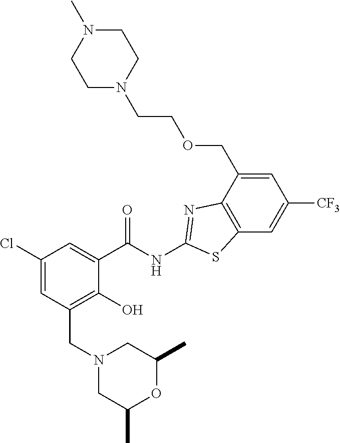 [SMILES: C[C@@H]1CN(Cc2cc(Cl)cc(C(=O)Nc3nc4c(COCCN5CCN(C)CC5)cc(C(F)(F)F)cc4s3)c2O)C[C@H](C)O1] | 5-chloro-3-((cis-2,6- dimethylmorpholino)methyl)-2-hydroxy-N-(4- ((2-(4-methylpiperazin-1-yl)ethoxy)methyl)-6- (trifluoromethyl)benzo[d]thiazol-2- yl)benzamide |

| | | |
|---|---|---|
| 77 | 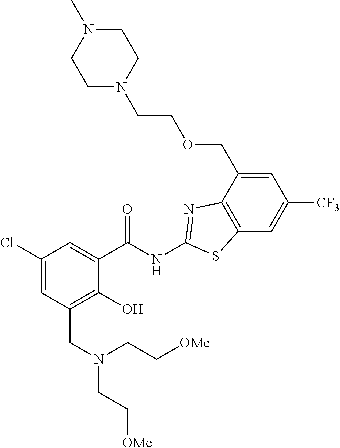 [SMILES: COCCN(CCOC)Cc1cc(Cl)cc(C(=O)Nc2nc3c(COCCN4CCN(C)CC4)cc(C(F)(F)F)cc3s2)c1O] | 3-((bis(2-methoxyethyl)amino)methyl)-5- chloro-2-hydroxy-N-(4-((2-(4-methylpiperazin- 1-yl)ethoxy)methyl)-6- (trifluoromethyl)benzo[d]thiazol-2- yl)benzamide |

| | | |
|---|---|---|
| 82 | 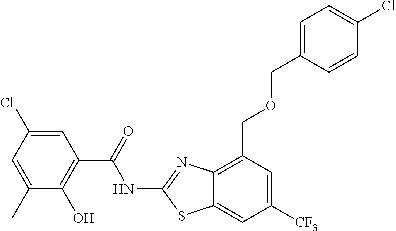 [SMILES: Cc1cc(Cl)cc(C(=O)Nc2nc3c(COCc4ccc(Cl)cc4)cc(C(F)(F)F)cc3s2)c1O] | 5-chloro-N-(4-(((4-chlorobenzyl)oxy)methyl)- 6-(trifluoromethyl)benzo[d]thiazol-2-yl)-2- hydroxy-3-methylbenzamide |

| | | |
|---|---|---|
| 83 | 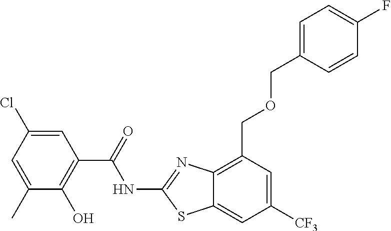 [SMILES: Cc1cc(Cl)cc(C(=O)Nc2nc3c(COCc4ccc(F)cc4)cc(C(F)(F)F)cc3s2)c1O] | 5-chloro-N-(4-(((4-fluorobenzyl)oxy)methyl)- 6-(trifluoromethyl)benzo[d]thiazol-2-yl)-2- hydroxy-3-methylbenzamide |

| | | |
|---|---|---|
| 84 | 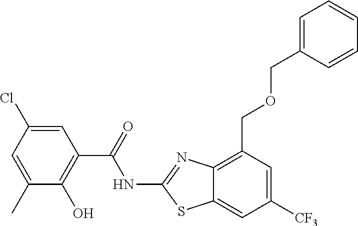 [SMILES: Cc1cc(Cl)cc(C(=O)Nc2nc3c(COCc4ccccc4)cc(C(F)(F)F)cc3s2)c1O] | N-(4-((benzyloxy)methyl)-6- (trifluoromethyl)benzo[d]thiazol-2-yl)-5- chloro-2-hydroxy-3-methylbenzamide |
| 85 | 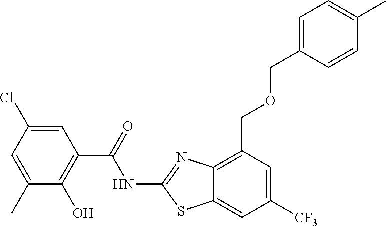 [SMILES: Cc1ccc(COCc2cc(C(F)(F)F)cc3sc(NC(=O)c4cc(Cl)cc(C)c4O)nc23)cc1] | 5-chloro-2-hydroxy-3-methyl-N-(4-(((4- methylbenzyl)oxy)methyl)-6- (trifluoromethyl)benzo[d]thiazol-2- yl)benzamide |
| 86 | 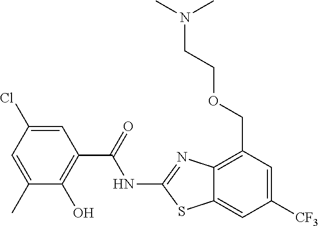 [SMILES: Cc1cc(Cl)cc(C(=O)Nc2nc3c(COCCN(C)C)cc(C(F)(F)F)cc3s2)c1O] | 5-chloro-N-(4-((2- (dimethylamino)ethoxy)methyl)-6- (trifluoromethyl)benzo[d]thiazol-2-yl)-2- hydroxy-3-methylbenzamide |
| 87 | 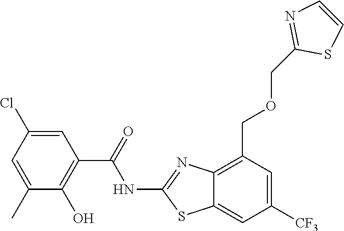 [SMILES: Cc1cc(Cl)cc(C(=O)Nc2nc3c(COCc4nccs4)cc(C(F)(F)F)cc3s2)c1O] | 5-chloro-2-hydroxy-3-methyl-N-(4-((thiazol-2- ylmethoxy)methyl)-6- (trifluoromethyl)benzo[d]thiazol-2- yl)benzamide |
| 88 | 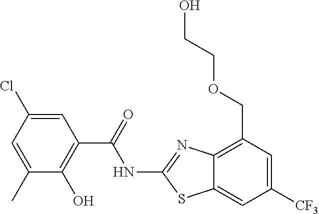 [SMILES: Cc1cc(Cl)cc(C(=O)Nc2nc3c(COCCO)cc(C(F)(F)F)cc3s2)c1O] | 5-chloro-2-hydroxy-N-(4-((2- hydroxyethoxy)methyl)-6- (trifluoromethyl)benzo[d]thiazol-2-yl)-3- methylbenzamide |
| 89 | 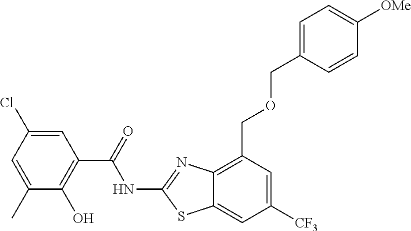 [SMILES: COc1ccc(COCc2cc(C(F)(F)F)cc3sc(NC(=O)c4cc(Cl)cc(C)c4O)nc23)cc1] | 5-chloro-2-hydroxy-N-(4-(((4- methoxybenzyl)oxy)methyl)-6- (trifluoromethyl)benzo[d]thiazol-2-yl)-3- methylbenzamide |
| 91 | 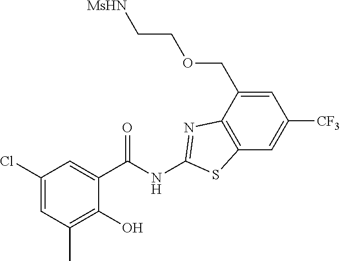 | 5-chloro-2-hydroxy-3-methyl-N-(4-((2- (methylsulfonamido)ethoxy)methyl)-6- (trifluoromethyl)benzo[d]thiazol-2- yl)benzamide |
| 92 | 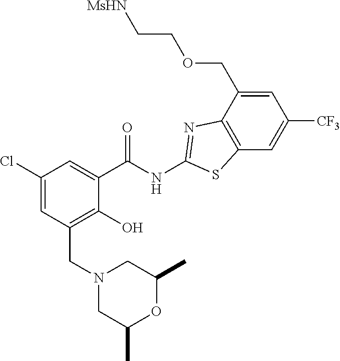 | 5-chloro-3-((cis-dimethylmorpholino)methyl)- 2-hydroxy-N-(4-((2- (methylsulfonamido)ethoxy)methyl)-6- (trifluoromethyl)benzo[d]thiazol-2- yl)benzamide |
| 93 | 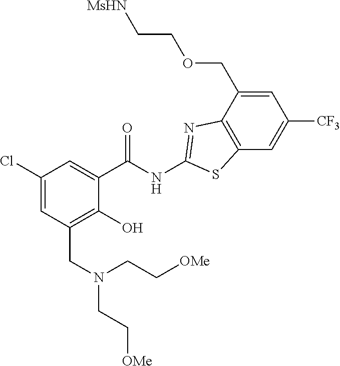 | 3-((bis(2-methoxyethyl)amino)methyl)-5- chloro-2-hydroxy-N-(4-((2- (methylsulfonamido)ethoxy)methyl)-6- (trifluoromethyl)benzo[d]thiazol-2- yl)benzamide |
| 100 | 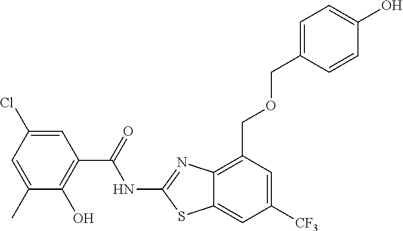 [SMILES: Cc1cc(Cl)cc(C(=O)Nc2nc3c(COCc4ccc(O)cc4)cc(C(F)(F)F)cc3s2)c1O] | 5-chloro-2-hydroxy-N-(4-(((4- hydroxybenzyl)oxy)methyl)-6- (trifluoromethyl)benzo[d]thiazol-2-yl)-3- methylbenzamide |
| 102 | 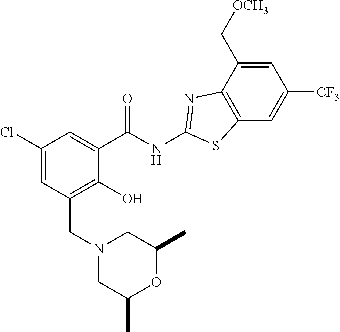 [SMILES: COCc1cc(C(F)(F)F)cc2sc(NC(=O)c3cc(Cl)cc(CN4C[C@@H](C)O[C@@H](C)C4)c3O)nc12] | 5-chloro-3-((cis-2,6- dimethylmorpholino)methyl)-2-hydroxy-N-(4- (methoxymethyl)-6- (trifluoromethyl)benzo[d]thiazol-2- yl)benzamide |
| 103 | 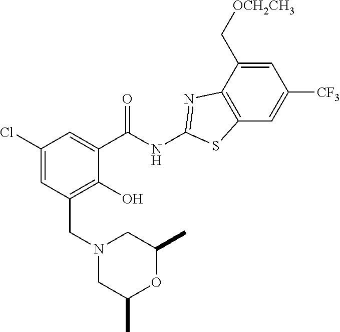 [SMILES: CCOCc1cc(C(F)(F)F)cc2sc(NC(=O)c3cc(Cl)cc(CN4C[C@@H](C)O[C@@H](C)C4)c3O)nc12] | 5-chloro-3-((cis-2,6- dimethylmorpholino)methyl)-N-(4- (ethoxymethyl)-6- (trifluoromethyl)benzo[d]thiazol-2-yl)-2- hydroxybenzamide |
| 104 | 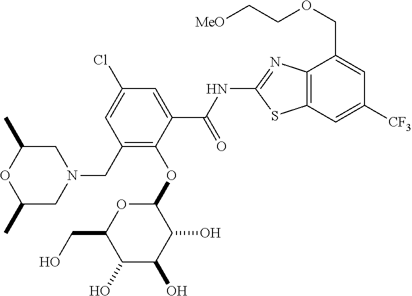 | 5-chloro-3-((cis-2,6- dimethylmorpholino)methyl)-N-(4-((2- methoxyethoxy)methyl)-6- (trifluoromethyl)benzo[d]thiazol-2-yl)-2- (((2S,3R,4S,5S,6R)-3,4,5-trihydroxy-6- (hydroxymethyl)tetrahydro-2H-pyran-2- yl)oxy)benzamide |
| 105 | 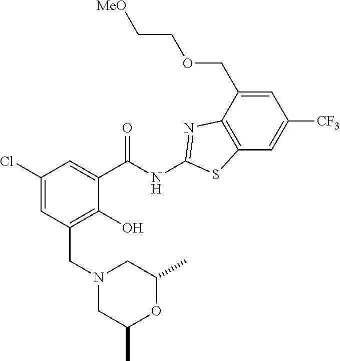 [SMILES: COCCOCc1cc(C(F)(F)F)cc2sc(NC(=O)c3cc(Cl)cc(CN4C[C@H](C)O[C@@H](C)C4)c3O)nc12] | 5-chloro-3-((trans-2,6- dimethylmorpholino)methyl)-2-hydroxy-N-(4- ((2-methoxyethoxy)methyl)-6- (trifluoromethyl)benzo[d]thiazol-2- yl)benzamide |
| 106 | 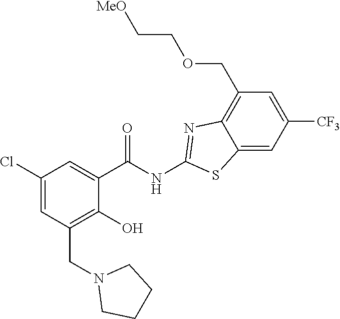 [SMILES: COCCOCc1cc(C(F)(F)F)cc2sc(NC(=O)c3cc(Cl)cc(CN4CCCC4)c3O)nc12] | 5-chloro-2-hydroxy-N-(4-((2- methoxyethoxy)methyl)-6- (trifluoromethyl)benzo[d]thiazol-2-yl)-3- (pyrrolidin-1-ylmethyl)benzamide |
| 107 | 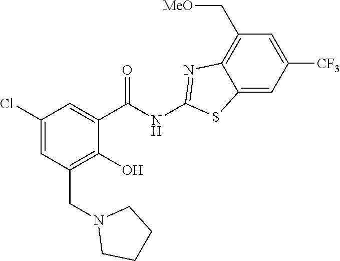 [SMILES: COCc1cc(C(F)(F)F)cc2sc(NC(=O)c3cc(Cl)cc(CN4CCCC4)c3O)nc12] | 5-chloro-2-hydroxy-N-(4-(methoxymethyl)-6- (trifluoromethyl)benzo[d]thiazol-2-yl)-3- (pyrrolidin-1-ylmethyl)benzamide |
| 108 | 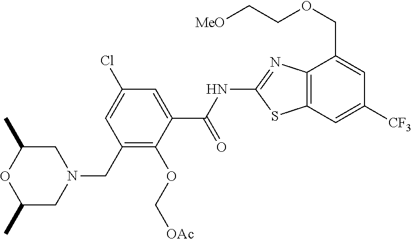 [SMILES: COCCOCc1cc(C(F)(F)F)cc2sc(NC(=O)c3cc(Cl)cc(CN4C[C@@H](C)O[C@@H](C)C4)c3OCOC(C)=O)nc12] | (4-chloro-2-((cis-2,6- dimethylmorpholino)methyl)-6-((4-((2- methoxyethoxy)methyl)-6- (trifluoromethyl)benzo[d]thiazol-2- yl)carbamoyl)phenoxy)methyl acetate |

or a pharmaceutically acceptable salt, solvate, or prodrug thereof

Some embodiments describe a compound selected from the group consisting of:

| | | |
|---|---|---|
| 64 | 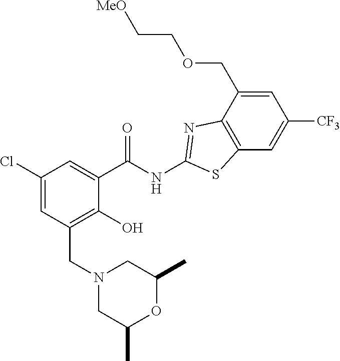 [SMILES: COCCOCc1cc(C(F)(F)F)cc2sc(NC(=O)c3cc(Cl)cc(CN4C[C@@H](C)O[C@@H](C)C4)c3O)nc12] | 5-chloro-3-((cis-2,6- dimethylmorpholino)methyl)-2-hydroxy-N-(4- ((2-methoxyethoxy)methyl)-6- (trifluoromethyl)benzo[d]thiazol-2- yl)benzamide(64) 5-chloro-3-((cis-2,6- dimethylmorpholino)methyl)-2-hydroxy-N-(4- ((2-methoxyethoxy)methyl)-6- (trifluoromethyl)benzo[d]thiazol-2- yl)benzamide hydrochloride (64A) |
| 65 | 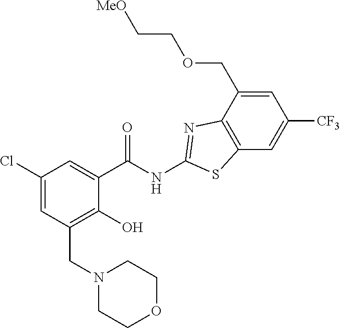 [SMILES: COCCOCc1cc(C(F)(F)F)cc2sc(NC(=O)c3cc(Cl)cc(CN4CCOCC4)c3O)nc12] | 5-chloro-2-hydroxy-N-(4-((2- methoxyethoxy)methyl)-6- (trifluoromethyl)benzo[d]thiazol-2-yl)-3- (morpholinomethyl)benzamide |
| 66 | 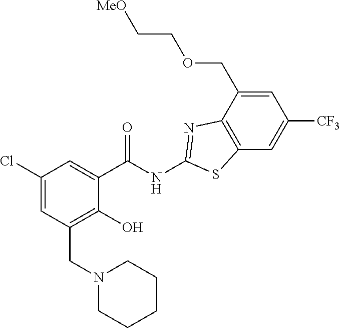 [SMILES: COCCOCc1cc(C(F)(F)F)cc2sc(NC(=O)c3cc(Cl)cc(CN4CCCCC4)c3O)nc12] | 5-chloro-2-hydroxy-N-(4-((2- methoxyethoxy)methyl)-6- (trifluoromethyl)benzo[d]thiazol-2-yl)-3- (piperidin-1-ylmethyl)benzamide |
| 69 | 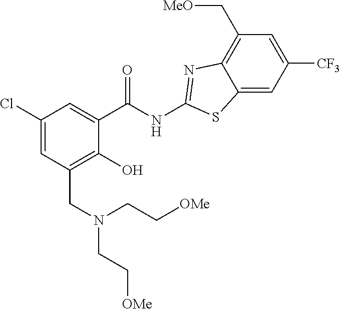 [SMILES: COCCN(CCOC)Cc1cc(Cl)cc(C(=O)Nc2nc3c(COC)cc(C(F)(F)F)cc3s2)c1O] | 3-((bis(2-methoxyethyl)amino)methyl)-5- chloro-2-hydroxy-N-(4-(methoxymethyl)-6- (trifluoromethyl)benzo[d]thiazol-2- yl)benzamide |
| 70 | 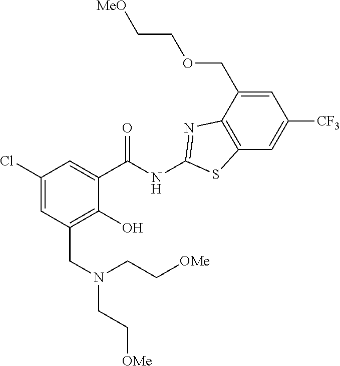 [SMILES: COCCOCc1cc(C(F)(F)F)cc2sc(NC(=O)c3cc(Cl)cc(CN(CCOC)CCOC)c3O)nc12] | 3-((bis(2-methoxyethyl)amino)methyl)-5- chloro-2-hydroxy-N-(4-((2- methoxyethoxy)methyl)-6- (trifluoromethyl)benzo[d]thiazol-2- yl)benzamide |
| 75 | 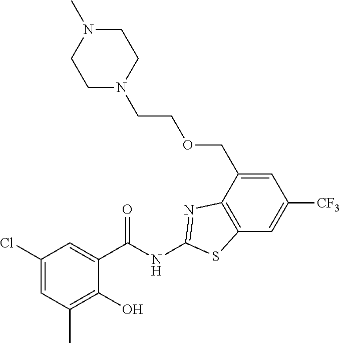 [SMILES: Cc1cc(Cl)cc(C(=O)Nc2nc3c(COCCN4CCN(C)CC4)cc(C(F)(F)F)cc3s2)c1O] | 5-chloro-2-hydroxy-3-methyl-N-(4-((2-(4- methylpiperazin-1-yl)ethoxy)methyl)-6- (trifluoromethyl)benzo[d]thiazol-2- yl)benzamide |
| 76 | 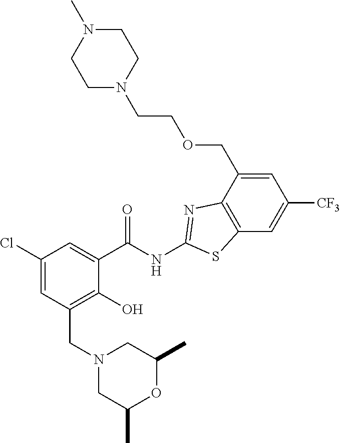 [SMILES: C[C@@H]1CN(Cc2cc(Cl)cc(C(=O)Nc3nc4c(COCCN5CCN(C)CC5)cc(C(F)(F)F)cc4s3)c2O)C[C@H](C)O1] | 5-chloro-3-((cis-2,6- dimethylmorpholino)methyl)-2-hydroxy-N-(4- ((2-(4-methylpiperazin-1-yl)ethoxy)methyl)-6- (trifluoromethyl)benzo[d]thiazol-2- yl)benzamide |
| 91 | 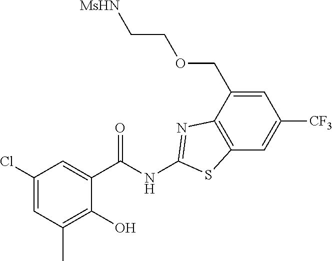 | 5-chloro-2-hydroxy-3-methyl-N-(4-((2- (methylsulfonamido)ethoxy)methyl)-6- (trifluoromethyl)benzo[d]thiazol-2- yl)benzamide |
| 92 | 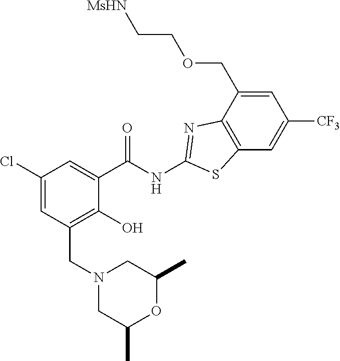 | 5-chloro-3-((cis-2,6- dimethylmorpholino)methyl)-2-hydroxy-N-(4- ((2-(methylsulfonamido)ethoxy)methyl)-6- (trifluoromethyl)benzo[d]thiazol-2- yl)benzamide |
| 93 | 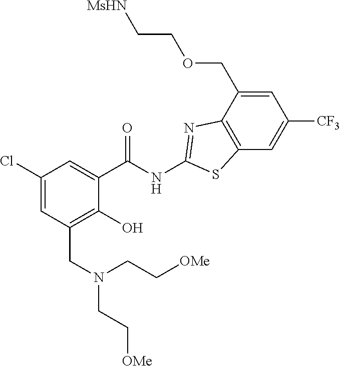 | 3-((bis(2-methoxyethyl)amino)methyl)-5- chloro-2-hydroxy-N-(4-((2- (methylsulfonamido)ethoxy)methyl)-6- (trifluoromethyl)benzo[d]thiazol-2- yl)benzamide |

or a pharmaceutically acceptable salt, solvate, or prodrug thereof.

| | | |
|---|---|---|
| 64 | 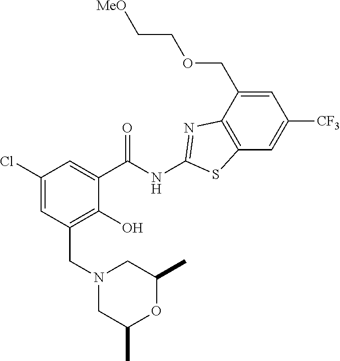 [SMILES: COCCOCc1cc(C(F)(F)F)cc2sc(NC(=O)c3cc(Cl)cc(CN4C[C@@H](C)O[C@@H](C)C4)c3O)nc12] | 5-chloro-3-((cis-2,6- dimethylmorpholino)methyl)-2-hydroxy-N-(4- ((2-methoxyethoxy)methyl)-6- (trifluoromethyl)benzo[d]thiazol-2- yl)benzamide |

or a pharmaceutically acceptable salt, solvate, or prodrug thereof.

In one embodiment, the present disclosure describes a compound of Formula II:

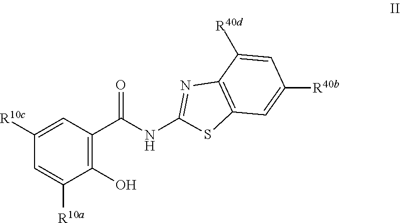

or a pharmaceutically acceptable salt, solvate, or prodrug thereof.

The substituent R^{10a} of Formula I is selected from the group consisting of OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)NHCO₂CH₃,

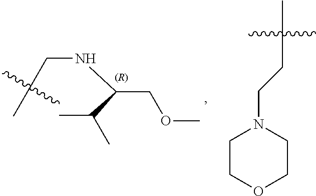

—(CH₂)ₒNR^{50A}R^{50B}, —(CH₂)ₒR⁶⁰, and C(O)N(CH₂CH₂OCH₃)₂. Each of substituents R^{50A} and R^{50B}, is independently selected from the group consisting of —C₁-C₆ alkyl; and —C₁-C₆ alkyl substituted with one or more groups selected from —C₁-C₆ alkoxy, and —O(CH₂)₂OCH₃, with the proviso that R^{50A} and R^{50B} are not both C₁-C₆ alkyl; alternatively R^{50A} and R^{50B} together with the nitrogen to which they are attached, form a 4 to 8-membered heterocyclyl optionally substituted with one or more substituents, independently selected from the group consisting of oxo, cyano, hydroxyl, alkoxy, acylamino, carboxyamido, —SO₂CH₃, —CF₃, C₁-C₆ alkyl, halo, and acyl. Integer o′ is an integer selected the group consisting of 1, 2 and 3. Substituent R⁶⁰ is selected from the group consisting 5 to 6-membered heterocyclyl, pyridinyl and thiazolyl; and o is an integer selected from the group consisting of 0 1, 2 and 3.

Substituent R^{10c} of Formula I is selected from the group consisting of chloro, fluoro, iodo, and bromo.

One of substituent R^{40b} and R^{40d} is H and the other substituent is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl.

Further embodiments describe the compound according to Formula IIa:

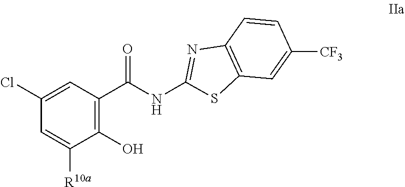

or a pharmaceutically acceptable salt, solvate, or prodrug thereof, wherein each of R^{10a}, and R^{40d} of Formula IIa is as described previously for Formula II.

In some embodiments, R^{10a} of Formula II, or of Formula IIa or a pharmaceutically acceptable salt, solvate, or prodrug thereof, is selected from —CH₂NR^{50A}R^{50B}; wherein each of R^{50A} and R^{50B} is independently selected from methyl; and C₁-C₆ alkyl substituted with one or more groups selected from methoxy, and —O(CH₂)₂OCH₃.

In some embodiments, R^{10a} of Formula II, or of Formula IIa or a pharmaceutically acceptable salt, solvate, or prodrug thereof, is selected from the group consisting of —CH₂NR^{50A}R^{50B}; wherein R^{50A} and R^{50B} together with the nitrogen to which they are attached, form a 4 to 8-membered heterocyclyl optionally substituted with one or more substituents, independently selected from the group consisting of oxo, cyano, hydroxyl, alkoxy,

-acylamino, carboxyamido, —SO₂CH₃, —CF₃, C₁-C₆ alkyl, halo, and acyl. In some embodiments the 4-8-membered heterocyclyl is selected from the group consisting of azetindinyl, pyrrolidinyl, piperidinyl, piperazinyl, oxopiperazinyl, morpholinyl, dioxothiomorphylinyl, and azabicyclo[3.2.1]octanyl; wherein the heterocyclyl is optionally substituted with one or more substituents, independently selected from the group consisting of oxo, cyano, hydroxyl, alkoxy,
-acylamino, carboxyamido, —SO₂CH₃, —CF₃, C₁-C₆ alkyl, halo, and acyl. In some embodiments the 4-8-membered heterocyclyl is selected from the group consisting of azetindinyl, pyrrolidinyl, piperidinyl, piperazinyl, oxopiperazinyl, morpholinyl, and dioxothiomorphylinyl; wherein the heterocyclyl is optionally substituted with one or more substituents independently selected from the group consisting of oxo, cyano, hydroxyl, methoxy, —NHC(O)CH₃, —C(O)NH₂, —SO₂CH₃,
—CF₃, methyl, fluoro, and acetyl. In some embodiments the 4 to 8-membered heterocyclyl is selected from the group consisting of azetindinyl; pyrrolidinyl; pyrrolidinyl substituted with one or more substituents selected from the group consisting of cyano, hydroxyl, methoxy, —NHC(O)CH₃, —C(O)NH₂, —SO₂CH₃, —CF₃ and methyl; piperidinyl; piperidinyl substituted with one or more substituents selected from the group consisting of —CF₃, and fluoro; piperazinyl substituted with one or more substituents selected from the group consisting of oxo, methyl, and acetyl; morpholinyl; morpholinyl substituted with one or more methyl groups; dioxothiomorphylinyl, and azabicyclo[3.2.1]octanyl. In some embodiments the 4 to 8-membered heterocyclyl is selected from the group consisting of azetindinyl; pyrrolidinyl; pyrrolidinyl substituted with one or more substituents selected from the group consisting of cyano, hydroxyl, methoxy, —NHC(O)CH₃, —C(O)NH₂, —SO₂CH₃, —CF₃ and methyl; piperidinyl; piperidinyl substituted with one or more substituents selected from the group consisting of —CF₃, and fluoro; piperazinyl substituted with one or more substituents selected from the group consisting of oxo, methyl, and acetyl; morpholinyl; morpholinyl substituted with one or more methyl groups; dioxothiomorphylinyl.

In some embodiments, R^{10a} of Formula II, or of Formula IIa or a pharmaceutically acceptable salt, solvate, or prodrug thereof, is selected from the group consisting of —CH₂NR^{50A}R^{50B}; wherein R^{50A} and R^{50B} together with the nitrogen to which they are attached, form a 4-7 membered heterocyclyl selected from the group consisting of azetindinyl, pyrrolidinyl, piperidinyl, piperazinyl, oxopiperazinyl, morpholinyl, and dioxothiomorphylinyl; wherein the 4-7 membered heterocyclyl is optionally substituted with one or more substituents independently selected from the group consisting of oxo, cyano, hydroxyl, methoxy, —NHC(O)CH₃, —C(O)NH₂, —SO₂CH₃, —CF₃, methyl, fluoro, and acetyl.

In some embodiments, R^{10a} of Formula II, or of Formula IIa or a pharmaceutically acceptable salt, solvate, or prodrug thereof, is selected from the group consisting of —CH₂NR^{50A}R^{50B}; wherein —CH₂NR^{50A}R^{50B} is selected from the group consisting of the group consisting of —CH₂N(CH₃)(CH₂)₂OCH₃, —(CH₂)N(CH₂CH₂OCH₃)₂, —CH₂N(CH₃)(CH₂)₂O(CH₂)₂OCH₃,

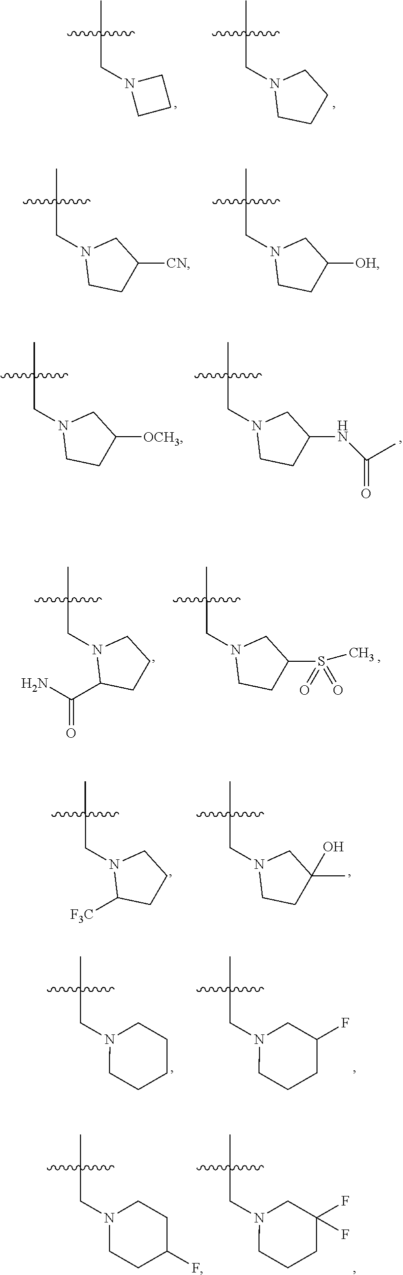

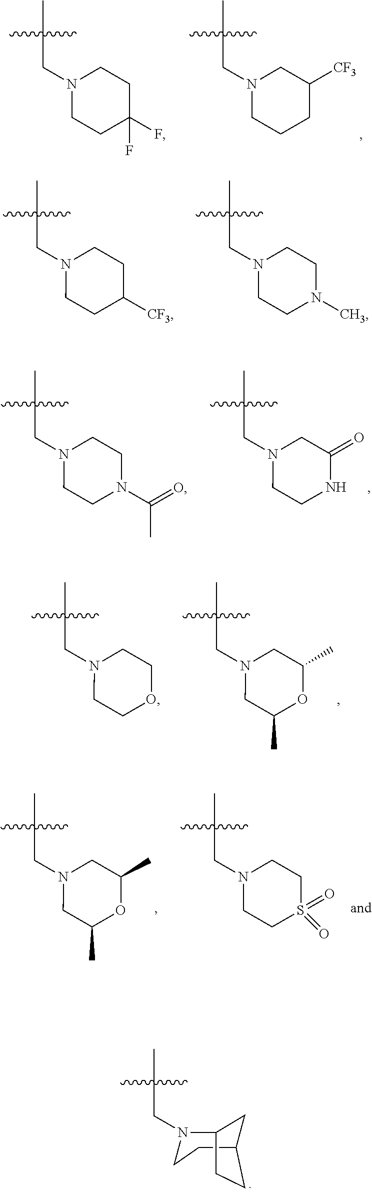

In some embodiments, R^{10a} of Formula II, or of Formula IIa or a pharmaceutically acceptable salt, solvate, or prodrug thereof, is selected from the group consisting of —CH₂NR^{50A}R^{50B}; wherein —CH₂NR^{50A}R^{50B} is selected from the group consisting of the group consisting of —CH₂N(CH₃)(CH₂)₂OCH₃, —(CH₂)N(CH₂CH₂OCH₃)₂, —CH₂N(CH₃)(CH₂)₂O(CH₂)₂OCH₃,

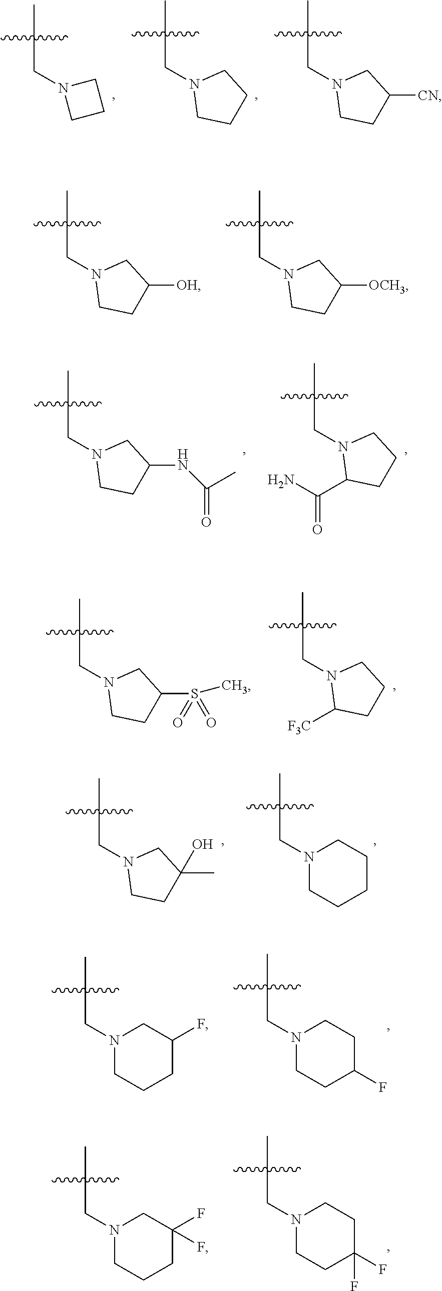

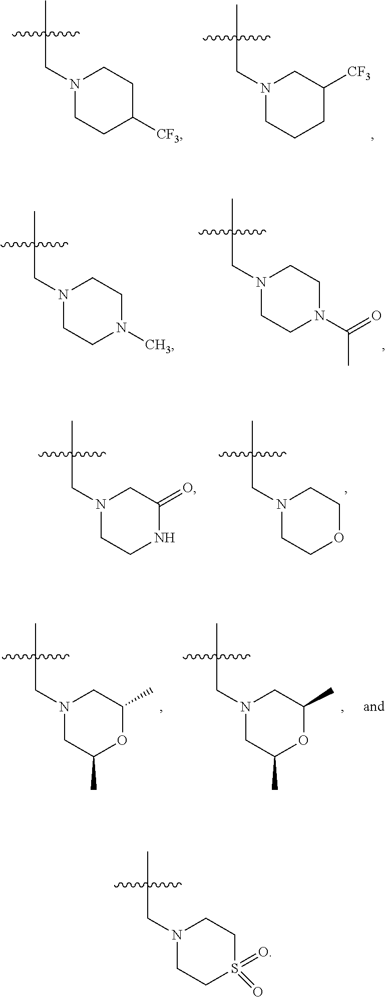

In some embodiments, R^{10a} of Formula II, or of Formula IIa or a pharmaceutically acceptable salt, solvate, or prodrug thereof, is —(CH₂)ₒR⁶⁰; wherein R⁶⁰ is selected from the group consisting of tetrahydrofuranyl, tetrahydropyranyl, piperidinyl, thiazolyl, and pyridinyl and o is as previously described. In some embodiments o is 0. In some embodiments o is 1. In some embodiments R⁶⁰ is selected from the group consisting of tetrahydrofuranyl, thiazolyl, and pyridinyl and o is 0. In some embodiments —(CH₂)ₒR⁶⁰ is selected from the group consisting of

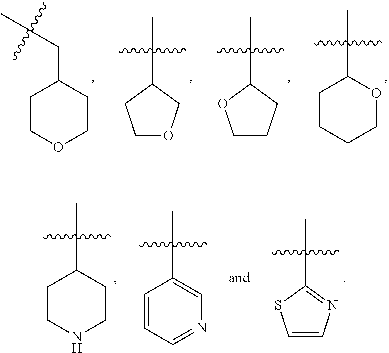

In some embodiments —(CH₂)ₒR⁶⁰ is selected from the group consisting of

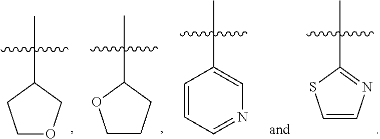

In some embodiments R^{10a} of Formula II, or of Formula IIa or a pharmaceutically acceptable salt, solvate, or prodrug thereof, is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

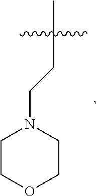 [SMILES: *CCN1CCOCC1]

—CH₂NR^{50A}R^{50B}, —(CH₂)ₒR⁶⁰, and C(O)N(CH₂CH₂OCH₃)₂, wherein R^{50A}, R^{50B}, and R⁶⁰ are as previously defined by any embodiment herein.

In some embodiments, R^{10a} of Formula II, or of Formula IIa or a pharmaceutically acceptable salt, solvate, or prodrug thereof, is —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

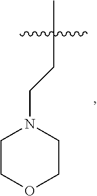 [SMILES: *CCN1CCOCC1]

—CH₂N(CH₃)(CH₂)₂OCH₃, —(CH₂)N(CH₂CH₂OCH₃)₂, —CH₂N(CH₃)(CH₂)₂O(CH₂)₂OCH₃,

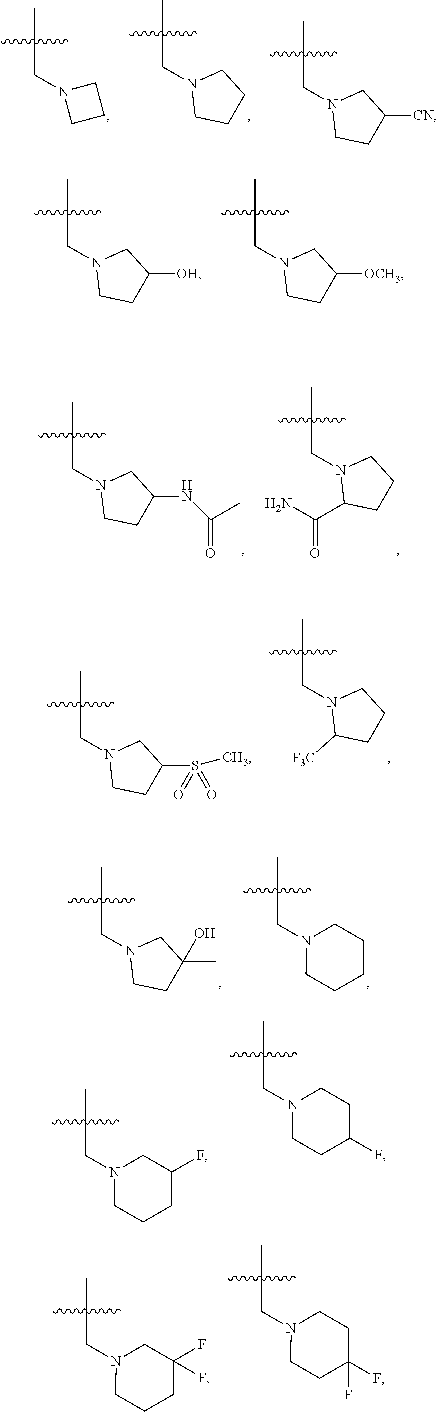

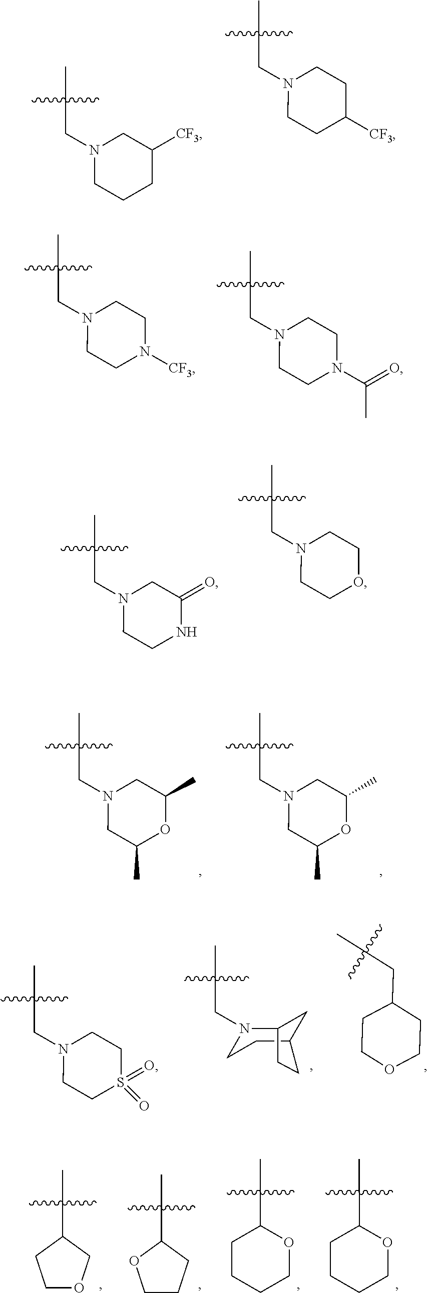

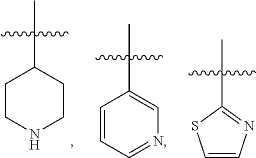

and C(O)N(CH₂CH₂OCH₃)₂.

In some embodiments, R^{10a} of Formula II, or of Formula IIa or a pharmaceutically acceptable salt, solvate, or prodrug thereof, is —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

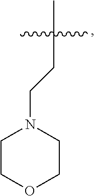 [SMILES: *CCN1CCOCC1]

—CH₂N(CH₃)(CH₂)₂OCH₃, —(CH₂)N(CH₂CH₂OCH₃)₂, —CH₂N(CH₃)(CH₂)₂O(CH₂)₂OCH₃,

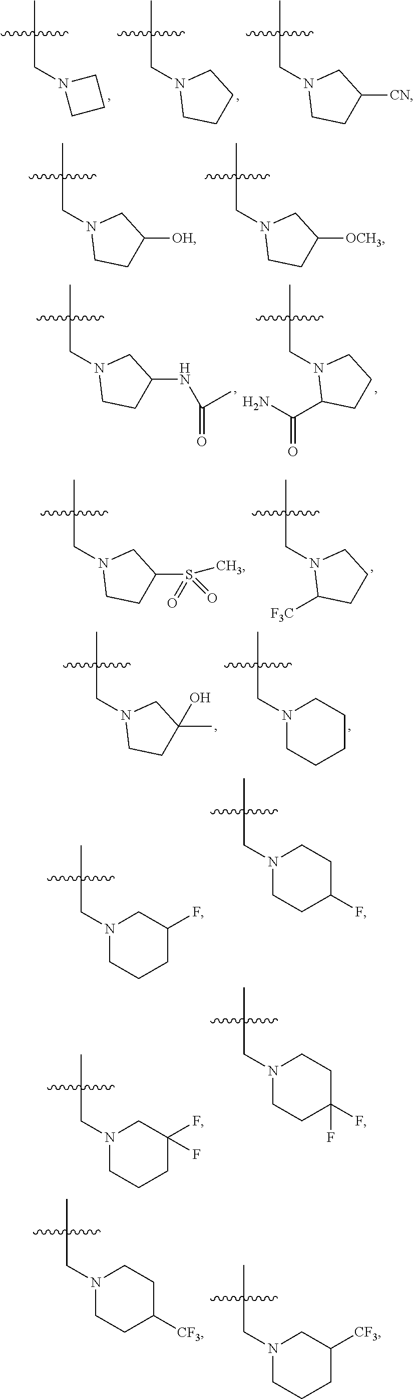

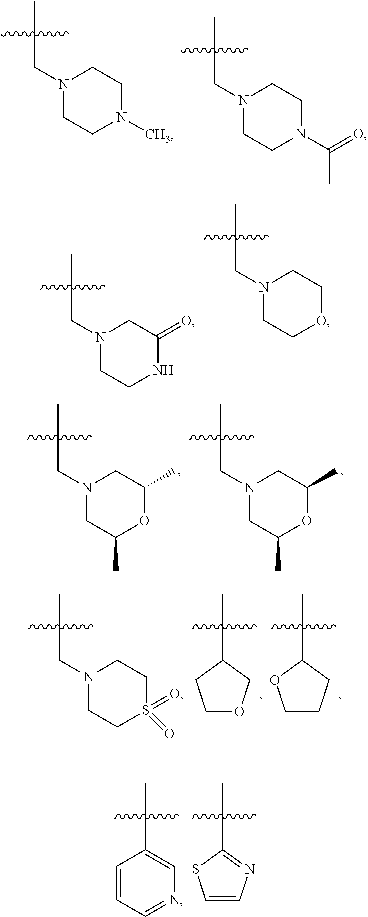

and C(O)N(CH₂CH₂OCH₃)₂.

In some embodiments, R^{10c} of Formula II, or a pharmaceutically acceptable salt, solvate, or prodrug thereof, is chloro, fluoro, and iodo.

In some embodiments, R^{10c} of Formula II, or a pharmaceutically acceptable salt, solvate, or prodrug thereof, is chloro.

In some embodiments one of substituent R^{40b} and R^{40d} is H and the other substituent is selected from the group consisting of fluoro, —CF₃, —CHF₂, and —OCF₃.

In some embodiments, R^{40b} is H and R^{40d} is selected from the group consisting of chloro, fluoro, iodo, bromo, —CF₃, —CHF₂, fluoro(C₁-C₆)alkyl, halo(C₁-C₆)alkyl, —OCF₃,

—SO₂(C₁-C₆)alkyl, cyano, and —CO₂(C₁-C₆)alkyl.

In some embodiments, R^{40d} is H and R^{40b} is selected from the group consisting of chloro, fluoro, iodo, bromo, —CF₃, —CHF₂, fluoro(C₁-C₆)alkyl, halo(C₁-C₆)alkyl, —OCF₃,

—SO₂(C₁-C₆)alkyl, cyano, and —CO₂(C₁-C₆)alkyl.

In some embodiments R^{40d} is H and R^{40b} is selected from the group consisting of fluoro, —CF₃, —CHF₂, and —OCF₃.

In some embodiments R^{40d} is H and R^{40b} is —CF₃.

Some embodiments describe a compound of Formula II or a pharmaceutically acceptable salt, solvate, or prodrug thereof, wherein
R^{10a} is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

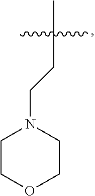 [SMILES: *CCN1CCOCC1]

CH₂NR^{50A}R^{50B}, —(CH₂)ₒR⁶⁰, and C(O)N(CH₂CH₂OCH₃)₂;
each of R^{50A} and R^{50B} is independently selected from the group consisting of —C₁-C₆ alkyl; and —C₁-C₆ alkyl substituted with one or more groups selected from —C₁-C₆ alkoxy, and —O(CH₂)₂OCH₃; alternatively R^{50A} and R^{50B} together with the nitrogen to which they are attached, form a 4 to 7-membered heterocyclyl optionally substituted with one or more substituents, independently selected from the group consisting of oxo, cyano, hydroxyl, alkoxy, acylamino, carboxyamido, —SO₂CH₃, —CF₃, C₁-C₆ alkyl, halo, and acyl;
R⁶⁰ is selected from the group consisting of 5 to 6-membered heterocyclyl, pyridinyl and thiazolyl;
o is 0;
R^{10c} is selected from the group consisting of chloro, fluoro, iodo; and
one of R^{40b} and R^{40d} is H and the other of R^{40b} and R^{40d} is selected from the group consisting of fluoro, —CF₃, —CHF₂, —OCF₃;
with the proviso that R^{50A} and R^{50B} are not both C₁-C₆ alkyl.

Some embodiments describe a compound of Formula II or a pharmaceutically acceptable salt, solvate, or prodrug thereof, wherein
R^{10a} is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

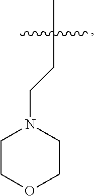 [SMILES: *CCN1CCOCC1]

—CH₂NR^{50A}R^{50B}, —(CH₂)ₒR⁶⁰, and C(O)N(CH₂CH₂OCH₃)₂;
each of R^{50A} and R^{50B} is independently selected from methyl; and C₁-C₆ alkyl substituted with one or more groups selected from methoxy, and —O(CH₂)₂OCH₃; alternatively R^{50A} and R^{50B} together with the nitrogen to which they are attached, form a 4-7-membered heterocyclyl selected from the group consisting of azetindinyl, pyrrolidinyl, piperidinyl, piperazinyl, oxopiperazinyl, morpholinyl, and dioxothiomorphylinyl; wherein the heterocyclyl is optionally substituted with one or more substituents independently selected from the group consisting of oxo, cyano, hydroxyl, methoxy, —NHC(O)CH₃, —C(O)NH₂, —SO₂CH₃, —CF₃, methyl, fluoro, and acetyl;
R⁶⁰ is selected from the group consisting of tetrahydrofuranyl, thiazolyl, and pyridinyl
o is 0;
R^{10c} is selected from the group consisting of chloro, fluoro, iodo; and
R^{40d} is H and R^{40b} is selected from the group consisting of fluoro, —CF₃, —CHF₂, and —OCF₃;
   with the proviso that R^{50A} and R^{50B} are not both methyl.

Some embodiments describe a compound of Formula II or a pharmaceutically acceptable salt, solvate, or prodrug thereof, wherein
R^{10a} is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

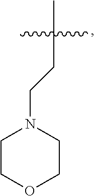 [SMILES: *CCN1CCOCC1]

—CH₂N(CH₃)(CH₂)₂OCH₃, —(CH₂)N(CH₂CH₂OCH₃)₂, —CH₂N(CH₃)(CH₂)₂O(CH₂)₂OCH₃,

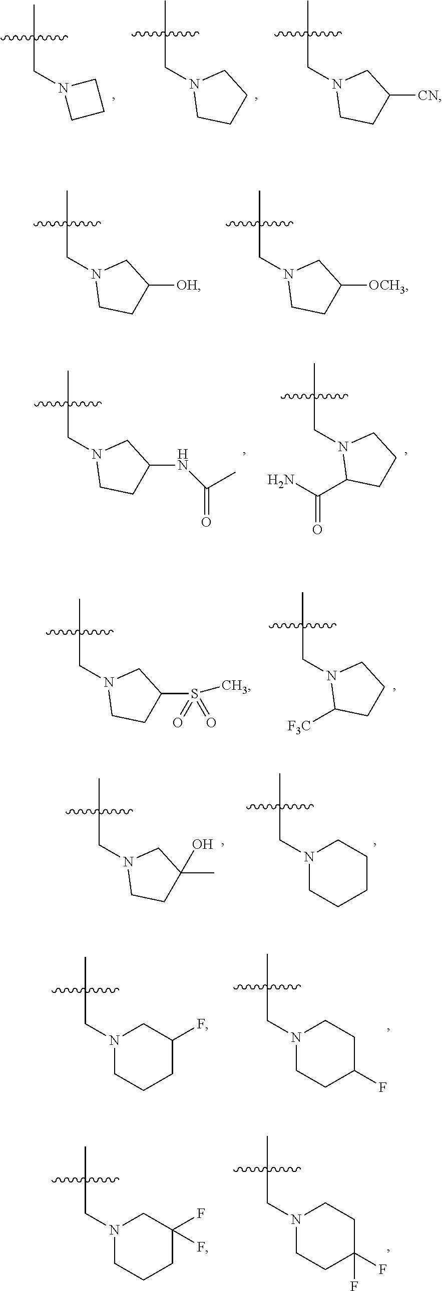

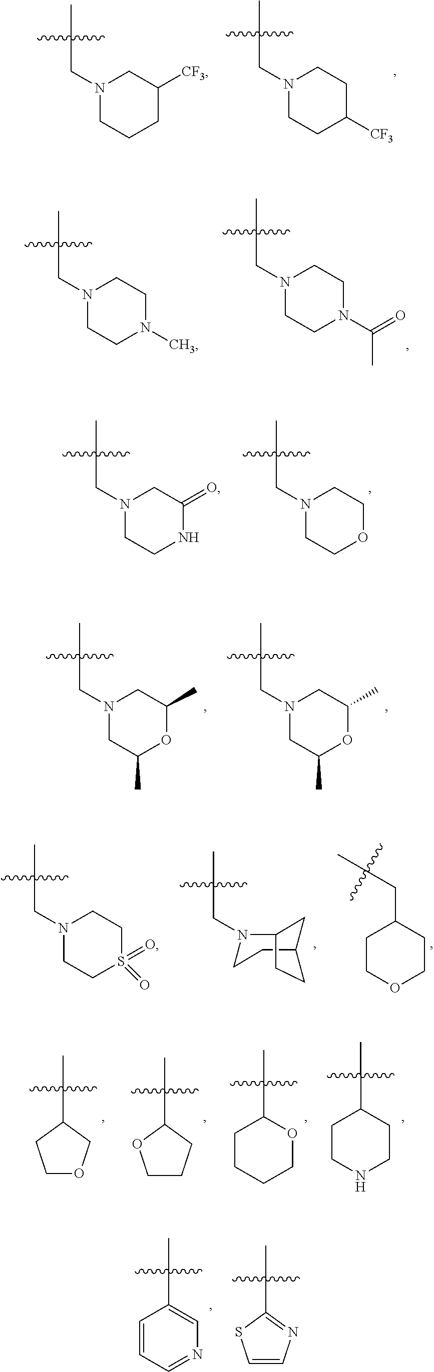

and
C(O)N(CH₂CH₂OCH₃)₂;
R^{10c} is selected from the group consisting of chloro, fluoro, iodo; and
R^{40d} is H and R^{40b} is selected from the group consisting of fluoro, —CF₃, —CHF₂, and —OCF₃.

Some embodiments describe a compound of Formula II or a pharmaceutically acceptable salt, solvate, or prodrug thereof, wherein
R^{10a} is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

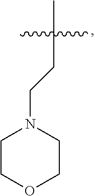 [SMILES: *CCN1CCOCC1]

—CH₂N(CH₃)(CH₂)₂OCH₃, —(CH₂)N(CH₂CH₂OCH₃)₂, —CH₂N(CH₃)(CH₂)₂O(CH₂)₂OCH₃,

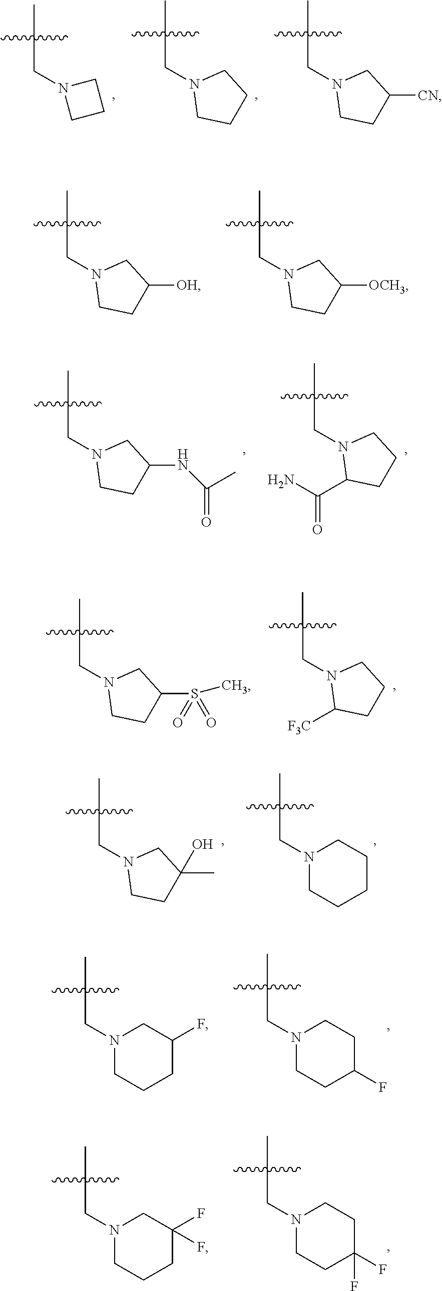

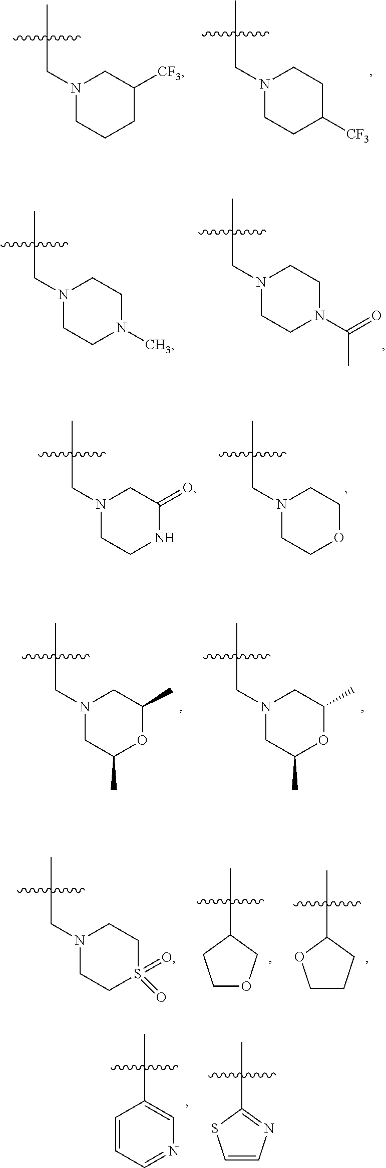

and C(O)N(CH₂CH₂OCH₃)₂;
R^{10c} is selected from the group consisting of chloro, fluoro, iodo; and
R^{40d} is H and R^{40b} is selected from the group consisting of fluoro, —CF₃, —CHF₂, and —OCF₃.

Some embodiments describe a compound of Formula II or a pharmaceutically acceptable salt, solvate, or prodrug thereof, wherein R^{10a} is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

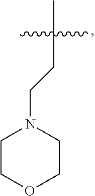 [SMILES: *CCN1CCOCC1]

CH₂N(CH₃)(CH₂)₂OCH₃, —(CH₂)N(CH₂CH₂OCH₃)₂, —CH₂N(CH₃)(CH₂)₂O(CH₂)₂OCH₃,

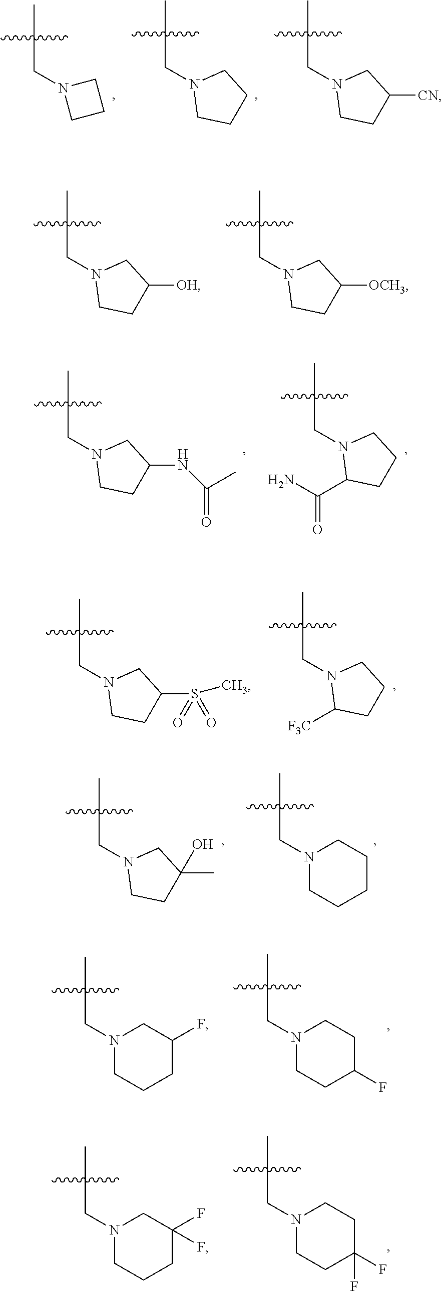

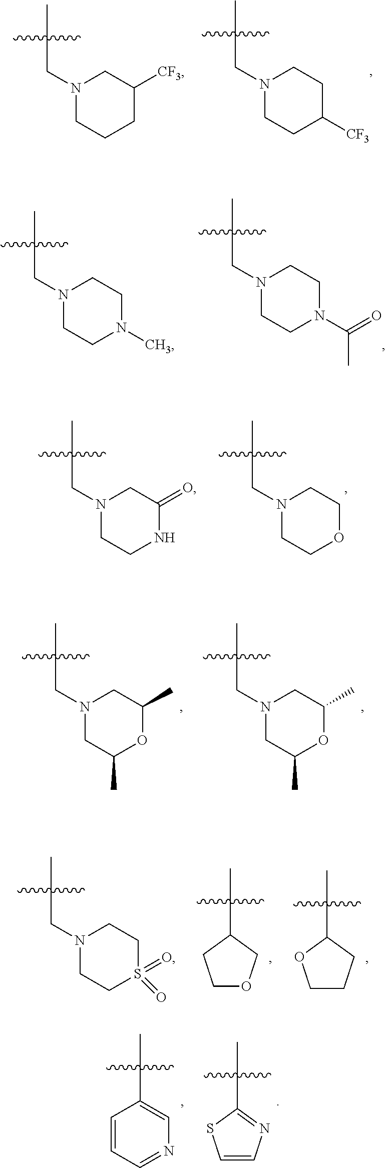

and C(O)N(CH₂CH₂OCH₃)₂;
R^{10c} is chloro; and
R^{40d} is H and R^{40b} is —CF₃.

Some embodiments describe a compound of Formula IIa or a pharmaceutically acceptable salt, solvate, or prodrug thereof, wherein
R^{10a} is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

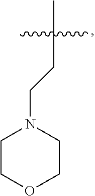 [SMILES: *CCN1CCOCC1]

—CH₂N(CH₃)(CH₂)₂OCH₃, —(CH₂)N(CH₂CH₂OCH₃)₂, —CH₂N(CH₃)(CH₂)₂O(CH₂)₂OCH₃,

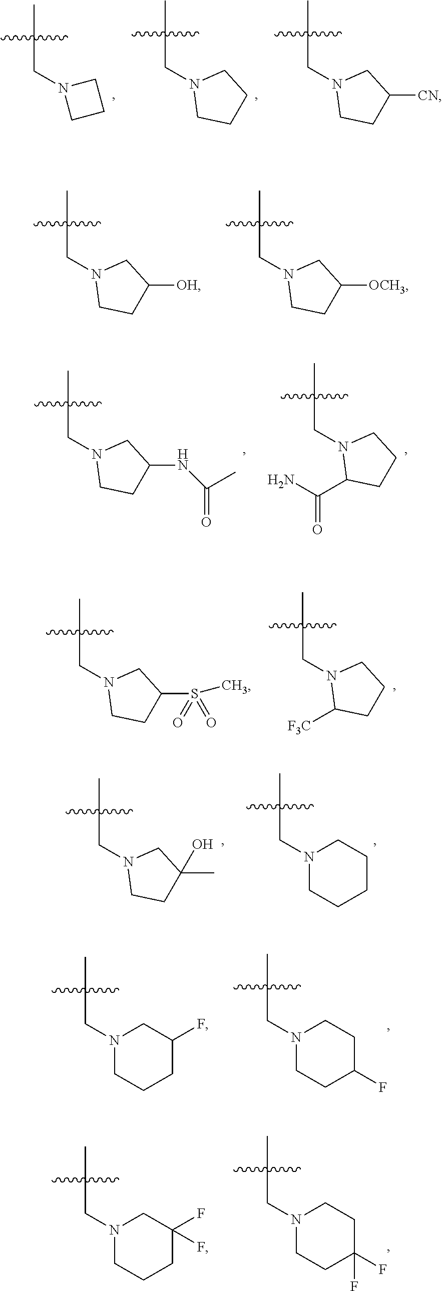

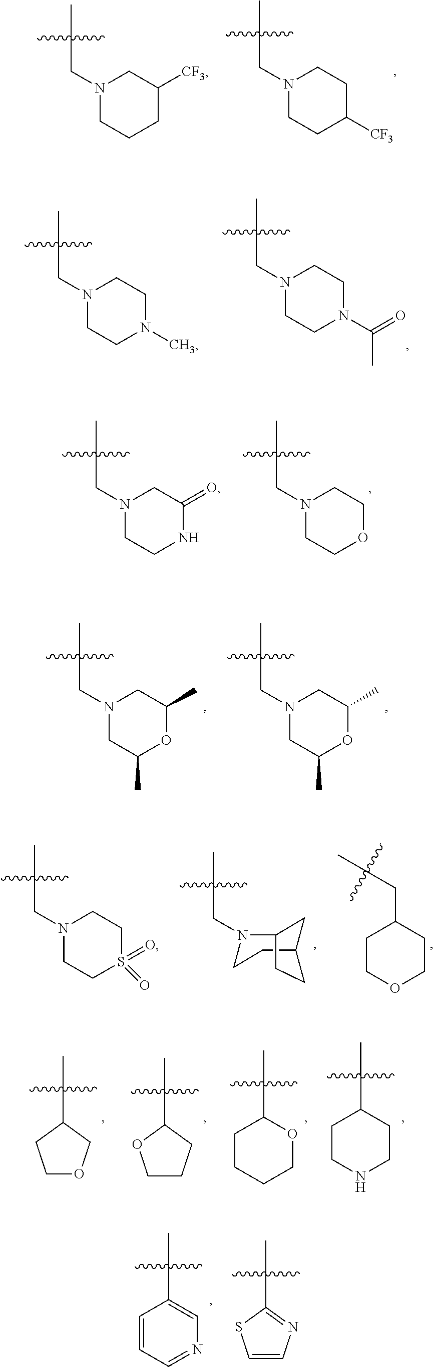

and
C(O)N(CH₂CH₂OCH₃)₂.

Some embodiments describe a compound of Formula IIa or a pharmaceutically acceptable salt, solvate, or prodrug thereof, wherein
R^{10a} is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃,

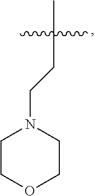 [SMILES: *CCN1CCOCC1]

CH₂N(CH₃)(CH₂)₂OCH₃, —(CH₂)N(CH₂CH₂OCH₃)₂, —CH₂N(CH₃)(CH₂)₂O(CH₂)₂OCH₃,

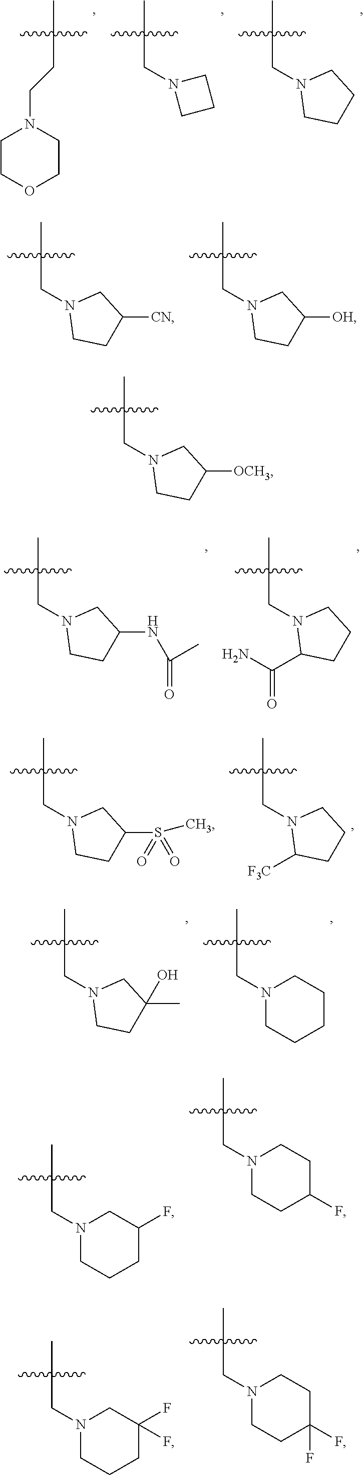

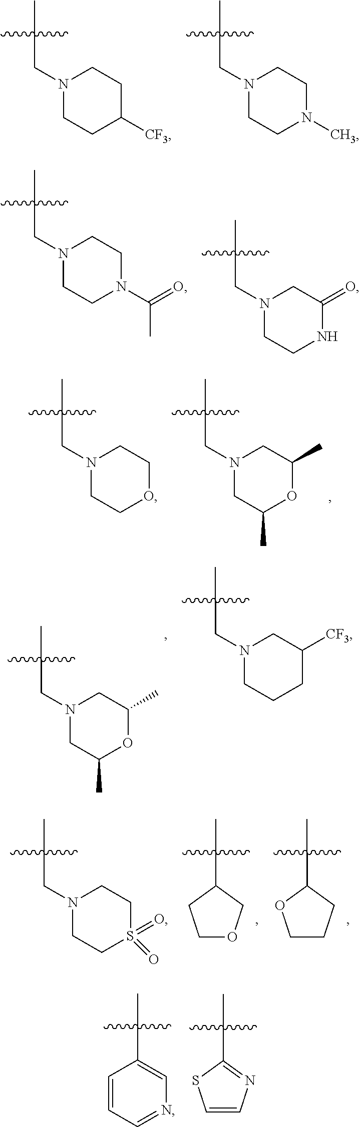

and C(O)N(CH₂CH₂OCH₃)₂.

Some embodiments describe a compound selected from the group consisting of:

| Number | Structure | Name |
|---|---|---|
| 1007 | 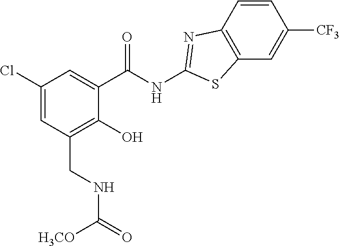 [SMILES: COC(=O)NCc1cc(Cl)cc(C(=O)Nc2nc3ccc(C(F)(F)F)cc3s2)c1O] | methyl (5-chloro-2-hydroxy-3-((6- (trifluoromethyl)benzo[d]thiazol-2- yl)carbamoyl)benzyl)carbamate |
| 1008 | 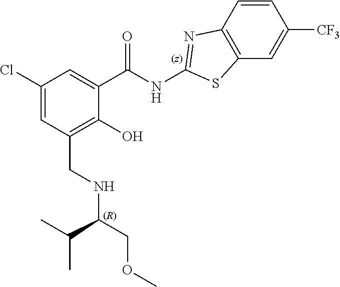 | (R)-5-chloro-2-hydroxy-3-(((1-methoxy-3- methylbutan-2-yl)amino)methyl)-N-(6- (trifluoromethyl)benzo[d]thiazol-2- yl)benzamide |
| 1009 | 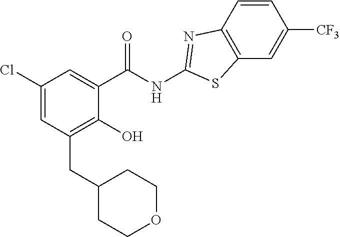 [SMILES: O=C(Nc1nc2ccc(C(F)(F)F)cc2s1)c1cc(Cl)cc(CC2CCOCC2)c1O] | 5-chloro-2-hydroxy-3-((tetrahydro-2H-pyran- 4-yl)methyl)-N-(6- (trifluoromethyl)benzo[d]thiazol-2- yl)benzamide |
| 1010 | 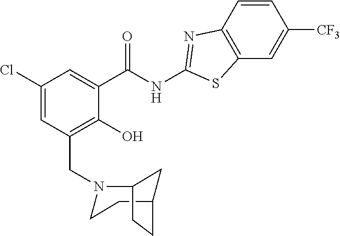 [SMILES: O=C(Nc1nc2ccc(C(F)(F)F)cc2s1)c1cc(Cl)cc(CN2CCC3CCC2C3)c1O] | 3-(((1R,5R)-2-azabicyclo[3.2.1]octan-2- yl)methyl)-5-chloro-2-hydroxy-N-(6- (trifluoromethyl)benzo[d]thiazol-2- yl)benzamide |
| 1011 | 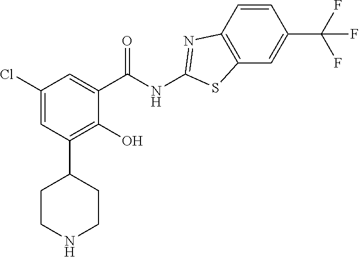 [SMILES: O=C(Nc1nc2ccc(C(F)(F)F)cc2s1)c1cc(Cl)cc(C2CCNCC2)c1O] | 5-chloro-2-hydroxy-3-(piperidin-4-yl)-N-(6- (trifluoromethyl)benzo[d]thiazol-2- yl)benzamide |
| 1012 | 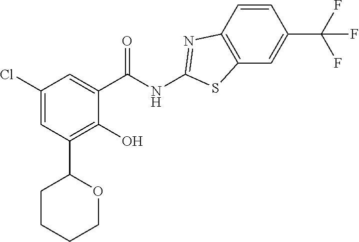 [SMILES: O=C(Nc1nc2ccc(C(F)(F)F)cc2s1)c1cc(Cl)cc(C2CCCCO2)c1O] | 5-chloro-2-hydroxy-3-(tetrahydro-2H-pyran-2- yl)-N-(6-(trifluoromethyl)benzo[d]thiazol-2- yl)benzamide |
| 1 | 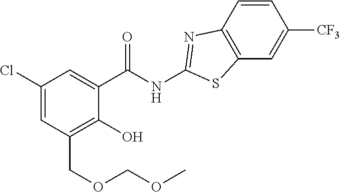 [SMILES: COCOCc1cc(Cl)cc(C(=O)Nc2nc3ccc(C(F)(F)F)cc3s2)c1O] | 5-chloro-2-hydroxy-3- ((methoxymethoxy)methyl)-N-(6- (trifluoromethyl)benzo[d]thiazol-2- yl)benzamide |
| 2 | 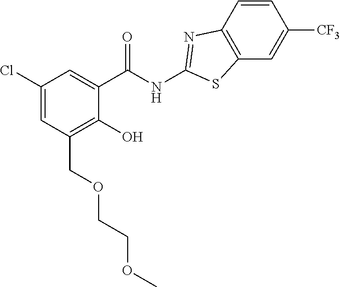 [SMILES: COCCOCc1cc(Cl)cc(C(=O)Nc2nc3ccc(C(F)(F)F)cc3s2)c1O] | 5-chloro-2-hydroxy-3-((2- methoxyethoxy)methyl)-N-(6- (trifluoromethyl)benzo[d]thiazol-2- yl)benzamide |
| 3 and 3A | 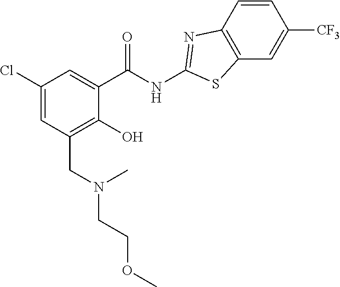 [SMILES: COCCN(C)Cc1cc(Cl)cc(C(=O)Nc2nc3ccc(C(F)(F)F)cc3s2)c1O] | 5-chloro-2-hydroxy-3-(((2- methoxyethyl)(methyl)amino)methyl)-N-(6- (trifluoromethyl)benzo[d]thiazol-2- yl)benzamide (3) and 5-chloro-2-hydroxy-3-(((2- methoxyethyl)(methyl)amino)methyl)-N-(6- (trifluoromethyl)benzo[d]thiazol-2- yl)benzamide hydrochloride (3A) |
| 5 | 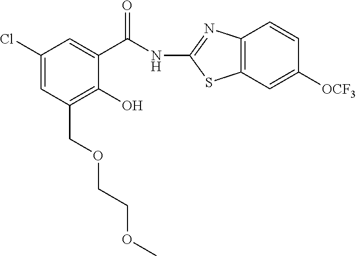 [SMILES: COCCOCc1cc(Cl)cc(C(=O)Nc2nc3ccc(OC(F)(F)F)cc3s2)c1O] | 5-chloro-2-hydroxy-3-((2- methoxyethoxy)methyl)-N-(6- (trifluoromethoxy)benzo[d]thiazol-2- yl)benzamide |
| 7 | 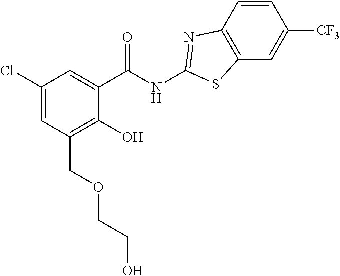 [SMILES: O=C(Nc1nc2ccc(C(F)(F)F)cc2s1)c1cc(Cl)cc(COCCO)c1O] | 5-chloro-2-hydroxy-3-((2- hydroxyethoxy)methyl)-N-(6- (trifluoromethyl)benzo[d]thiazol-2- yl)benzamide |
| 8 | 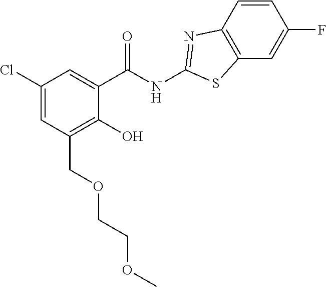 [SMILES: COCCOCc1cc(Cl)cc(C(=O)Nc2nc3ccc(F)cc3s2)c1O] | 5-chloro-N-(6-fluorobenzo[d]thiazol-2-yl)-2- hydroxy-3-((2- methoxyethoxy)methyl)benzamide |
| 9 | 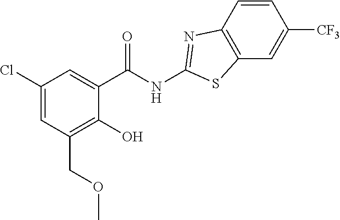 [SMILES: COCc1cc(Cl)cc(C(=O)Nc2nc3ccc(C(F)(F)F)cc3s2)c1O] | 5-chloro-2-hydroxy-3-(methoxymethyl)-N-(6- (trifluoromethyl)benzo[d]thiazol-2- yl)benzamide |
| 10 and 10A | 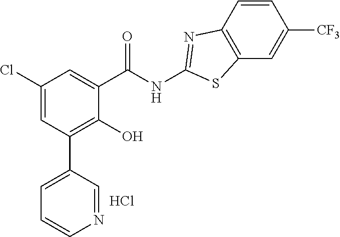 [SMILES: O=C(Nc1nc2ccc(C(F)(F)F)cc2s1)c1cc(Cl)cc(-c2cccnc2)c1O ; Cl] | 5-chloro-2-hydroxy-3-(pyridin-3-yl)-N-(6- (trifluoromethyl)benzo[d]thiazol-2- yl)benzamide hydrochloride(10A) 5-chloro-2-hydroxy-3-(pyridin-3-yl)-N-(6- (trifluoromethyl)benzo[d]thiazol-2- yl)benzamide(10) |
| 11 and 11A | 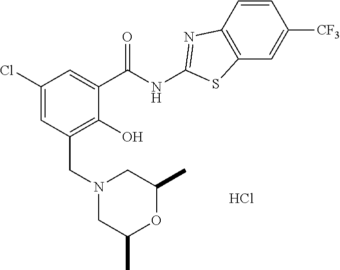 [SMILES: C[C@@H]1CN(Cc2cc(Cl)cc(C(=O)Nc3nc4ccc(C(F)(F)F)cc4s3)c2O)C[C@H](C)O1 ; Cl] | 5-chloro-3-((cis-2,6- dimethylmorpholino)methyl)-2-hydroxy-N-(6- (trifluoromethyl)benzo[d]thiazol-2- yl)benzamide hydrochloride (11A) 5-chloro-3-((cis-2,6- dimethylmorpholino)methyl)-2-hydroxy-N-(6- (trifluoromethyl)benzo[d]thiazol-2- yl)benzamide(11) |
| 12 and 12A | 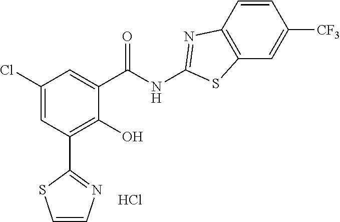 [SMILES: O=C(Nc1nc2ccc(C(F)(F)F)cc2s1)c1cc(Cl)cc(-c2nccs2)c1O ; Cl] | 5-chloro-2-hydroxy-3-(thiazol-2-yl)-N-(6- (trifluoromethyl)benzo[d]thiazol-2- yl)benzamide hydrochloride(12A) 5-chloro-2-hydroxy-3-(thiazol-2-yl)-N-(6- (trifluoromethyl)benzo[d]thiazol-2- yl)benzamide(12) |
| 14 | 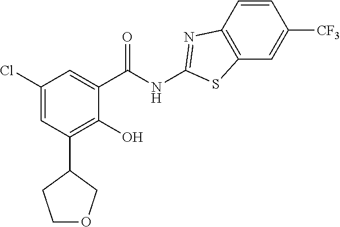 [SMILES: O=C(Nc1nc2ccc(C(F)(F)F)cc2s1)c1cc(Cl)cc(C2CCOC2)c1O] | 5-chloro-2-hydroxy-3-(tetrahydrofuran-3-yl)- N-(6-(trifluoromethyl)benzo[d]thiazol-2- yl)benzamide |
| 15 | 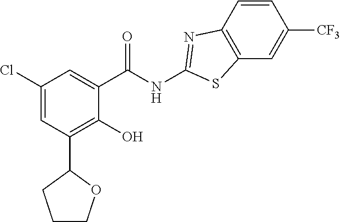 [SMILES: O=C(Nc1nc2ccc(C(F)(F)F)cc2s1)c1cc(Cl)cc(C2CCCO2)c1O] | 5-chloro-2-hydroxy-3-(tetrahydrofuran-2-yl)- N-(6-(trifluoromethyl)benzo[d]thiazol-2- yl)benzamide |
| 16 | 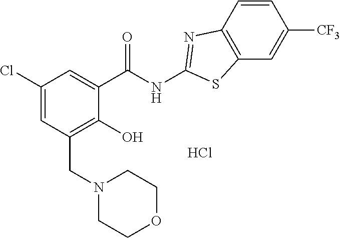 [SMILES: O=C(Nc1nc2ccc(C(F)(F)F)cc2s1)c1cc(Cl)cc(CN2CCOCC2)c1O ; Cl] | 5-chloro-2-hydroxy-3-(morpholinomethyl)-N- (6-(trifluoromethyl)benzo[d]thiazol-2- yl)benzamide hydrochloride(16A) 5-chloro-2-hydroxy-3-(morpholinomethyl)-N- (6-(trifluoromethyl)benzo[d]thiazol-2- yl)benzamide(16) |
| 17 | 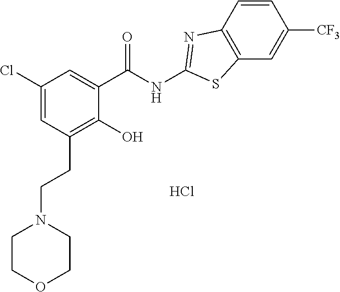 [SMILES: O=C(Nc1nc2ccc(C(F)(F)F)cc2s1)c1cc(Cl)cc(CCN2CCOCC2)c1O ; Cl] | 5-chloro-2-hydroxy-3-(2-morpholinoethyl)-N- (6-(trifluoromethyl)benzo[d]thiazol-2- yl)benzamide hydrochloride(17A) 5-chloro-2-hydroxy-3-(2-morpholinoethyl)-N- (6-(trifluoromethyl)benzo[d]thiazol-2- yl)benzamide(17) |
| 19 | 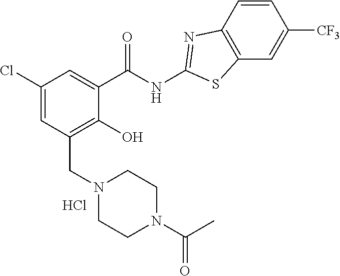 [SMILES: CC(=O)N1CCN(Cc2cc(Cl)cc(C(=O)Nc3nc4ccc(C(F)(F)F)cc4s3)c2O)CC1 ; Cl] | 3-((4-acetylpiperazin-1-yl)methyl)-5-chloro-2- hydroxy-N-(6- (trifluoromethyl)benzo[d]thiazol-2- yl)benzamide hydrochloride(19A) 3-((4-acetylpiperazin-1-yl)methyl)-5-chloro-2- hydroxy-N-(6- (trifluoromethyl)benzo[d]thiazol-2- yl)benzamide (19) |
| 22 | 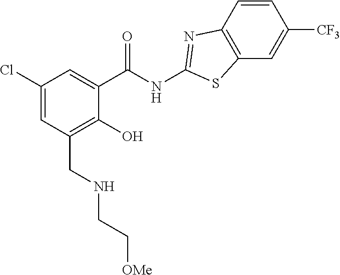 [SMILES: COCCNCc1cc(Cl)cc(C(=O)Nc2nc3ccc(C(F)(F)F)cc3s2)c1O] | 5-chloro-2-hydroxy-3-(((2- methoxyethyl)amino)methyl)-N-(6- (trifluoromethyl)benzo[d]thiazol-2- yl)benzamide hydrochloride |
| 23 | 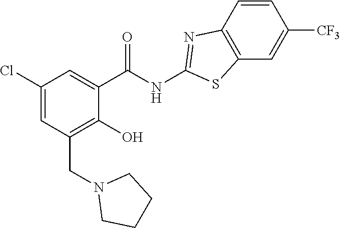 [SMILES: O=C(Nc1nc2ccc(C(F)(F)F)cc2s1)c1cc(Cl)cc(CN2CCCC2)c1O] | 5-chloro-2-hydroxy-3-(pyrrolidin-1-ylmethyl)- N-(6-(trifluoromethyl)benzo[d]thiazol-2- yl)benzamide |
| 24 | 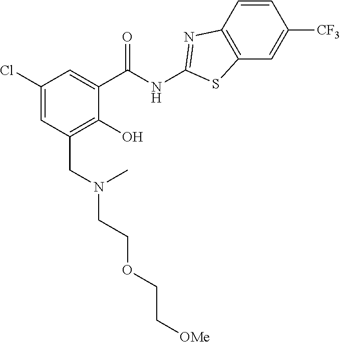 [SMILES: COCCOCCN(C)Cc1cc(Cl)cc(C(=O)Nc2nc3ccc(C(F)(F)F)cc3s2)c1O] | 5-chloro-2-hydroxy-3-(((2-(2- methoxyethoxy)ethyl)(methyl)amino)methyl)- N-(6-(trifluoromethyl)benzo[d]thiazol-2- yl)benzamide (24) 5-chloro-2-hydroxy-3-(((2-(2- methoxyethoxy)ethyl)(methyl)amino)methyl)- N-(6-(trifluoromethyl)benzo[d]thiazol-2- yl)benzamide hydrochloride (24A) |
| 25 | 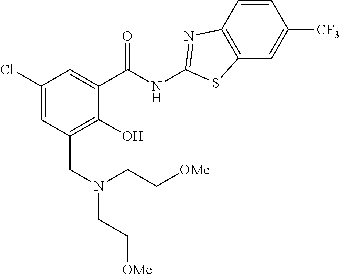 [SMILES: COCCN(CCOC)Cc1cc(Cl)cc(C(=O)Nc2nc3ccc(C(F)(F)F)cc3s2)c1O] | 3-((bis(2-methoxyethyl)amino)methyl)-5- chloro-2-hydroxy-N-(6- (trifluoromethyl)benzo[d]thiazol-2- yl)benzamide (25) 3-((bis(2-methoxyethyl)amino)methyl)-5- chloro-2-hydroxy-N-(6- (trifluoromethyl)benzo[d]thiazol-2- yl)benzamide hydrochloride (25A) |
| 26 | 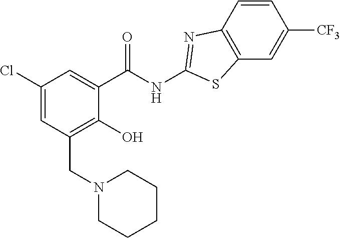 [SMILES: O=C(Nc1nc2ccc(C(F)(F)F)cc2s1)c1cc(Cl)cc(CN2CCCCC2)c1O] | 5-chloro-2-hydroxy-3-(piperidin-1-ylmethyl)- N-(6-(trifluoromethyl)benzo[d]thiazol-2- yl)benzamide |
| 27 | 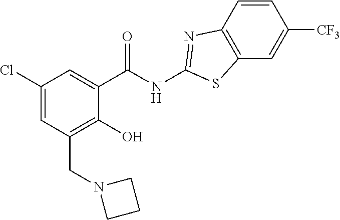 [SMILES: O=C(Nc1nc2ccc(C(F)(F)F)cc2s1)c1cc(Cl)cc(CN2CCC2)c1O] | 3-(azetidin-1-ylmethyl)-5-chloro-2-hydroxy-N- (6-(trifluoromethyl)benzo[d]thiazol-2- yl)benzamide |
| 28 | 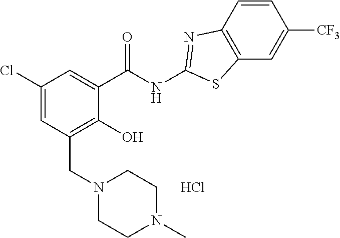 [SMILES: CN1CCN(Cc2cc(Cl)cc(C(=O)Nc3nc4ccc(C(F)(F)F)cc4s3)c2O)CC1 ; Cl] | 5-chloro-2-hydroxy-3-((4-methylpiperazin-1- yl)methyl)-N-(6- (trifluoromethyl)benzo[d]thiazol-2- yl)benzamide |
| 29 | 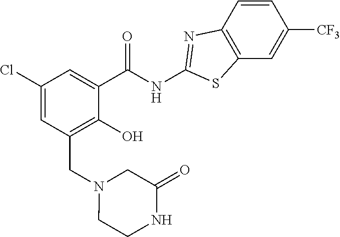 [SMILES: O=C1CN(Cc2cc(Cl)cc(C(=O)Nc3nc4ccc(C(F)(F)F)cc4s3)c2O)CCN1] | 5-chloro-2-hydroxy-3-((3-oxopiperazin-1- yl)methyl)-N-(6- (trifluoromethyl)benzo[d]thiazol-2- yl)benzamide |
| 30 | 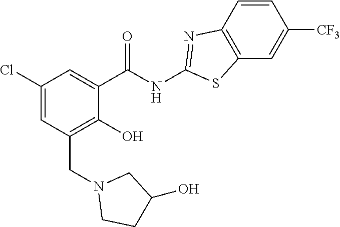 [SMILES: O=C(Nc1nc2ccc(C(F)(F)F)cc2s1)c1cc(Cl)cc(CN2CCC(O)C2)c1O] | 5-chloro-2-hydroxy-3-((3-hydroxypyrrolidin- 1-yl)methyl)-N-(6- (trifluoromethyl)benzo[d]thiazol-2- yl)benzamide |
| 31 | 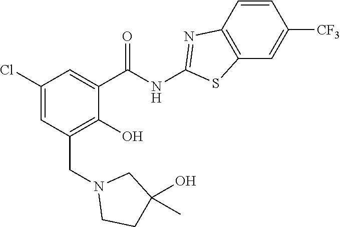 [SMILES: CC1(O)CCN(Cc2cc(Cl)cc(C(=O)Nc3nc4ccc(C(F)(F)F)cc4s3)c2O)C1] | 5-chloro-2-hydroxy-3-((3-hydroxy-3- methylpyrrolidin-1-yl)methyl)-N-(6- (trifluoromethyl)benzo[d]thiazol-2- yl)benzamide |
| 32 | 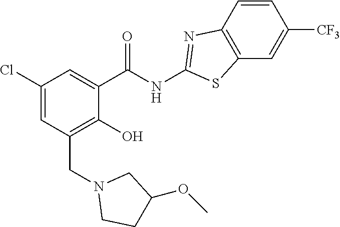 [SMILES: COC1CCN(Cc2cc(Cl)cc(C(=O)Nc3nc4ccc(C(F)(F)F)cc4s3)c2O)C1] | 5-chloro-2-hydroxy-3-((3-methoxypyrrolidin- 1-yl)methyl)-N-(6- (trifluoromethyl)benzo[d]thiazol-2- yl)benzamide |
| 33 | 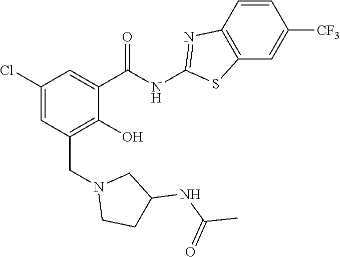 [SMILES: CC(=O)NC1CCN(Cc2cc(Cl)cc(C(=O)Nc3nc4ccc(C(F)(F)F)cc4s3)c2O)C1] | 3-((3-acetamidopyrrolidin-1-yl)methyl)-5- chloro-2-hydroxy-N-(6- (trifluoromethyl)benzo[d]thiazol-2- yl)benzamide |
| 34 | 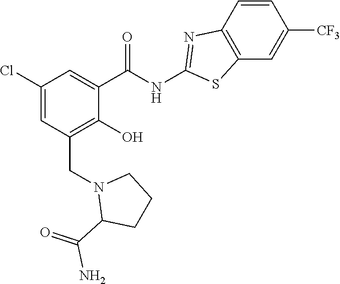 [SMILES: NC(=O)C1CCCN1Cc1cc(Cl)cc(C(=O)Nc2nc3ccc(C(F)(F)F)cc3s2)c1O] | 1-(5-chloro-2-hydroxy-3-((6- (trifluoromethyl)benzo[d]thiazol-2- yl)carbamoyl)benzyl)pyrrolidine-2- carboxamide |
| 35 | 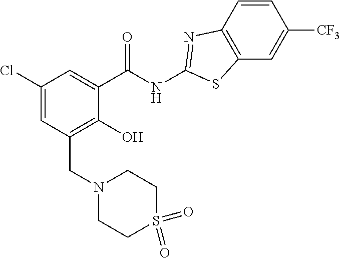 [SMILES: O=C(Nc1nc2ccc(C(F)(F)F)cc2s1)c1cc(Cl)cc(CN2CCS(=O)(=O)CC2)c1O] | 5-chloro-3-((1,1- dioxidothiomorpholino)methyl)-2-hydroxy-N- (6-(trifluoromethyl)benzo[d]thiazol-2- yl)benzamide |
| 36 | 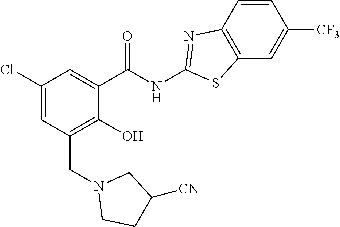 [SMILES: N#CC1CCN(Cc2cc(Cl)cc(C(=O)Nc3nc4ccc(C(F)(F)F)cc4s3)c2O)C1] | 5-chloro-3-((3-cyanopyrrolidin-1-yl)methyl)-2- hydroxy-N-(6- (trifluoromethyl)benzo[d]thiazol-2- yl)benzamide |
| 37 | 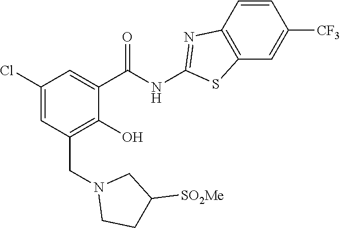 [SMILES: CS(=O)(=O)C1CCN(Cc2cc(Cl)cc(C(=O)Nc3nc4ccc(C(F)(F)F)cc4s3)c2O)C1] | 5-chloro-2-hydroxy-3-((3- (methylsulfonyl)pyrrolidin-1-yl)methyl)-N-(6- (trifluoromethyl)benzo[d]thiazol-2- yl)benzamide |
| 40 | 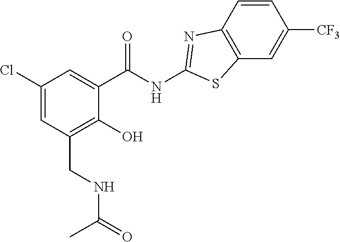 [SMILES: CC(=O)NCc1cc(Cl)cc(C(=O)Nc2nc3ccc(C(F)(F)F)cc3s2)c1O] | 3-(acetamidomethyl)-5-chloro-2-hydroxy-N- (6-(trifluoromethyl)benzo[d]thiazol-2- yl)benzamide |
| 41 | 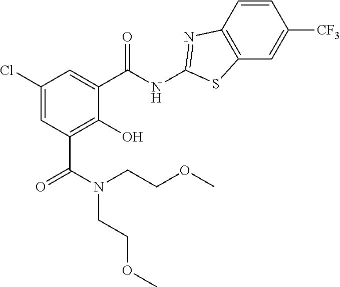 [SMILES: COCCN(CCOC)C(=O)c1cc(Cl)cc(C(=O)Nc2nc3ccc(C(F)(F)F)cc3s2)c1O] | 5-chloro-2-hydroxy-N1,N1-bis(2- methoxyethyl)-N3-(6- (trifluoromethyl)benzo[d]thiazol-2- yl)isophthalamide |
| 42 | 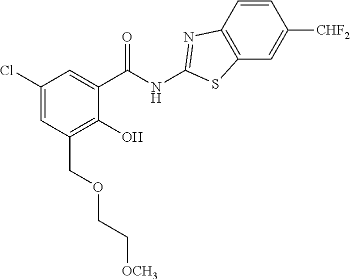 [SMILES: COCCOCc1cc(Cl)cc(C(=O)Nc2nc3ccc(C(F)F)cc3s2)c1O] | 5-chloro-N-(6- (difluoromethyl)benzo[d]thiazol-2-yl)-2- hydroxy-3-((2- methoxyethoxy)methyl)benzamide |
| 43 | 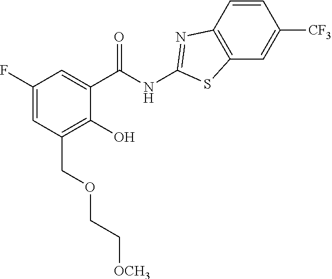 [SMILES: COCCOCc1cc(F)cc(C(=O)Nc2nc3ccc(C(F)(F)F)cc3s2)c1O] | 5-fluoro-2-hydroxy-3-((2- methoxyethoxy)methyl)-N-(6- (trifluoromethyl)benzo[d]thiazol-2- yl)benzamide |
| 46 | 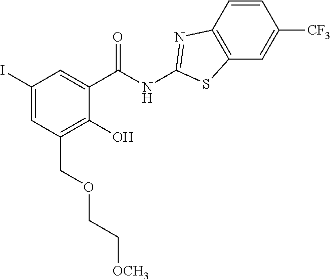 [SMILES: COCCOCc1cc(I)cc(C(=O)Nc2nc3ccc(C(F)(F)F)cc3s2)c1O] | 2-hydroxy-5-iodo-3-((2- methoxyethoxy)methyl)-N-(6- (trifluoromethyl)benzo[d]thiazol-2- yl)benzamide |
| 56 | 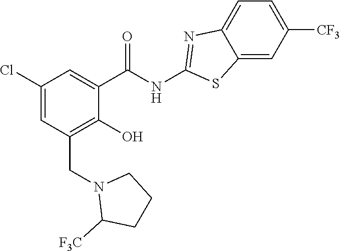 [SMILES: O=C(Nc1nc2ccc(C(F)(F)F)cc2s1)c1cc(Cl)cc(CN2CCCC2C(F)(F)F)c1O] | 5-chloro-2-hydroxy-N-(6- (trifluoromethyl)benzo[d]thiazol-2-yl)-3-((2- (trifluoromethyl)pyrrolidin-1- yl)methyl)benzamide (56) 5-chloro-2-hydroxy-N-(6- (trifluoromethyl)benzo[d]thiazol-2-yl)-3-((2- (trifluoromethyl)pyrrolidin-1- yl)methyl)benzamide hydrochloride(56A) |
| 57 | 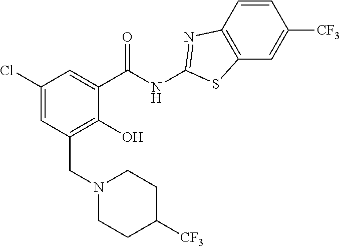 [SMILES: O=C(Nc1nc2ccc(C(F)(F)F)cc2s1)c1cc(Cl)cc(CN2CCC(C(F)(F)F)CC2)c1O] | 5-chloro-2-hydroxy-N-(6- (trifluoromethyl)benzo[d]thiazol-2-yl)-3-((4- (trifluoromethyl)piperidin-1- yl)methyl)benzamide |
| 58 | 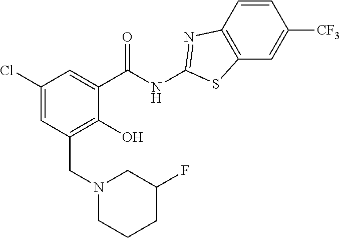 [SMILES: O=C(Nc1nc2ccc(C(F)(F)F)cc2s1)c1cc(Cl)cc(CN2CCCC(F)C2)c1O] | 5-chloro-3-((3-fluoropiperidin-1-yl)methyl)-2- hydroxy-N-(6- (trifluoromethyl)benzo[d]thiazol-2- yl)benzamide |
| 59 | 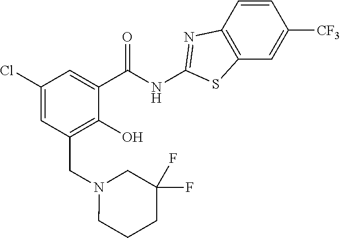 [SMILES: O=C(Nc1nc2ccc(C(F)(F)F)cc2s1)c1cc(Cl)cc(CN2CCCC(F)(F)C2)c1O] | 5-chloro-3-((3,3-difluoropiperidin-1- yl)methyl)-2-hydroxy-N-(6- (trifluoromethyl)benzo[d]thiazol-2- yl)benzamide |
| 60 | 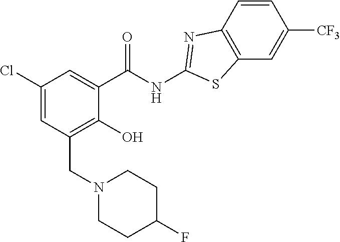 [SMILES: O=C(Nc1nc2ccc(C(F)(F)F)cc2s1)c1cc(Cl)cc(CN2CCC(F)CC2)c1O] | 5-chloro-3-((4-fluoropiperidin-1-yl)methyl)-2- hydroxy-N-(6- (trifluoromethyl)benzo[d]thiazol-2- yl)benzamide |
| 61 | 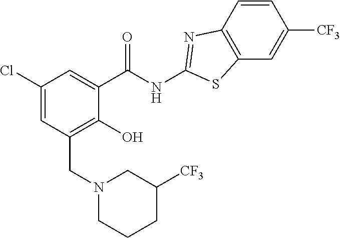 [SMILES: O=C(Nc1nc2ccc(C(F)(F)F)cc2s1)c1cc(Cl)cc(CN2CCCC(C(F)(F)F)C2)c1O] | 5-chloro-2-hydroxy-N-(6- (trifluoromethyl)benzo[d]thiazol-2-yl)-3-((3- (trifluoromethyl)piperidin-1- yl)methyl)benzamide |
| 62 | 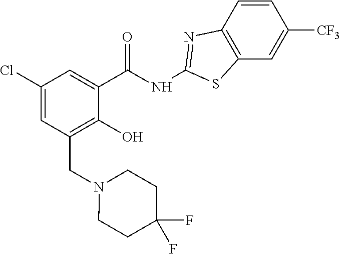 [SMILES: O=C(Nc1nc2ccc(C(F)(F)F)cc2s1)c1cc(Cl)cc(CN2CCC(F)(F)CC2)c1O] | 5-chloro-3-((4,4-difluoropiperidin-1- yl)methyl)-2-hydroxy-N-(6- (trifluoromethyl)benzo[d]thiazol-2- yl)benzamide |
| 97 | 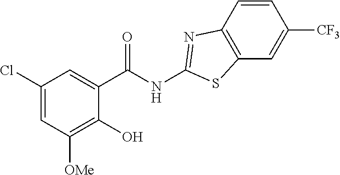 [SMILES: COc1cc(Cl)cc(C(=O)Nc2nc3ccc(C(F)(F)F)cc3s2)c1O] | 5-chloro-2-hydroxy-3-methoxy-N-(6- (trifluoromethyl)benzo[d]thiazol-2- yl)benzamide |

or a pharmaceutically acceptable salt, solvate, or prodrug thereof.

Some embodiments describe a compound selected from the group consisting of:

| Number | Structure | Name |
|---|---|---|
| 3 | 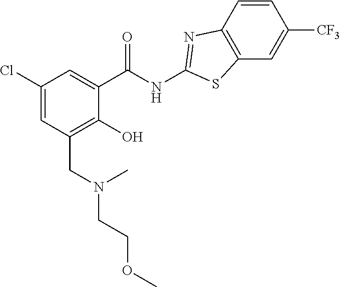 [SMILES: COCCN(C)Cc1cc(Cl)cc(C(=O)Nc2nc3ccc(C(F)(F)F)cc3s2)c1O] | 5-chloro-2-hydroxy-3-(((2- methoxyethyl)(methyl)amino)methyl)-N-(6- (trifluoromethyl)benzo[d]thiazol-2- yl)benzamide and 5-chloro-2-hydroxy-3-(((2- methoxyethyl)(methyl)amino)methyl)-N-(6- (trifluoromethyl)benzo[d]thiazol-2- yl)benzamide hydrochloride |
| 16 and 16(A) | 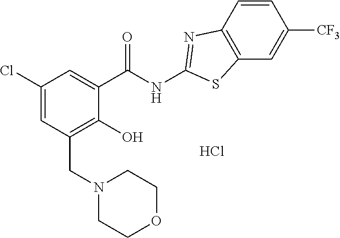 [SMILES: O=C(Nc1nc2ccc(C(F)(F)F)cc2s1)c1cc(Cl)cc(CN2CCOCC2)c1O ; Cl] | 5-chloro-2-hydroxy-3-(morpholinomethyl)-N- (6-(trifluoromethyl)benzo[d]thiazol-2- yl)benzamide hydrochloride (16A) 5-chloro-2-hydroxy-3-(morpholinomethyl)-N- (6-(trifluoromethyl)benzo[d]thiazol-2- yl)benzamide(16) |
| 17 and 17A | 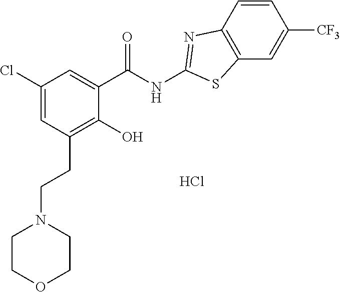 [SMILES: O=C(Nc1nc2ccc(C(F)(F)F)cc2s1)c1cc(Cl)cc(CCN2CCOCC2)c1O ; Cl] | 5-chloro-2-hydroxy-3-(2-morpholinoethyl)-N- (6-(trifluoromethyl)benzo[d]thiazol-2- yl)benzamide hydrochloride(17A) 5-chloro-2-hydroxy-3-(2-morpholinoethyl)-N- (6-(trifluoromethyl)benzo[d]thiazol-2- yl)benzamide(17) |
| 22 | 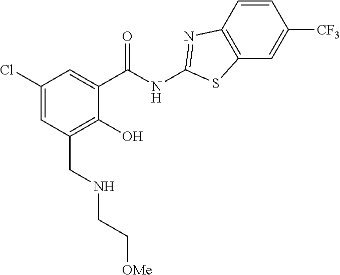 [SMILES: COCCNCc1cc(Cl)cc(C(=O)Nc2nc3ccc(C(F)(F)F)cc3s2)c1O] | 5-chloro-2-hydroxy-3-(((2- methoxyethyl)amino)methyl)-N-(6- (trifluoromethyl)benzo[d]thiazol-2- yl)benzamide hydrochloride |
| 23 | 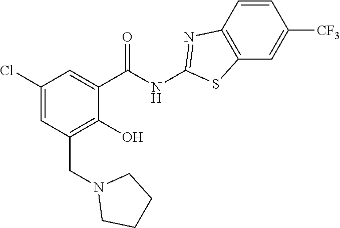 [SMILES: O=C(Nc1nc2ccc(C(F)(F)F)cc2s1)c1cc(Cl)cc(CN2CCCC2)c1O] | 5-chloro-2-hydroxy-3-(pyrrolidin-1-ylmethyl)- N-(6-(trifluoromethyl)benzo[d]thiazol-2- yl)benzamide |
| 24 | 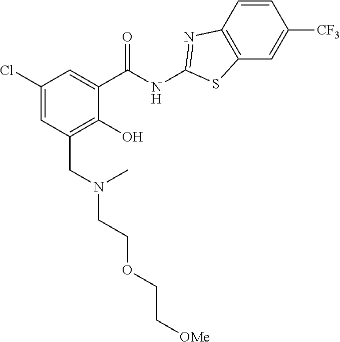 [SMILES: COCCOCCN(C)Cc1cc(Cl)cc(C(=O)Nc2nc3ccc(C(F)(F)F)cc3s2)c1O] | 5-chloro-2-hydroxy-3-(((2-(2- methoxyethoxy)ethyl)(methyl)amino)methyl)- N-(6-(trifluoromethyl)benzo[d]thiazol-2- yl)benzamide(24) 5-chloro-2-hydroxy-3-(((2-(2- methoxyethoxy)ethyl)(methyl)amino)methyl)- N-(6-(trifluoromethyl)benzo[d]thiazol-2- yl)benzamide hydrochloride (24A) |
| 25 | 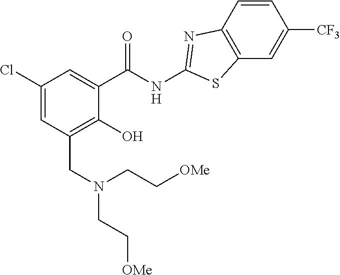 [SMILES: COCCN(CCOC)Cc1cc(Cl)cc(C(=O)Nc2nc3ccc(C(F)(F)F)cc3s2)c1O] | 3-((bis(2-methoxyethyl)amino)methyl)-5- chloro-2-hydroxy-N-(6- (trifluoromethyl)benzo[d]thiazol-2- yl)benzamide(25) 3-((bis(2-methoxyethyl)amino)methyl)-5- chloro-2-hydroxy-N-(6- (trifluoromethyl)benzo[d]thiazol-2- yl)benzamide hydrochloride (25A) |
| 26 | 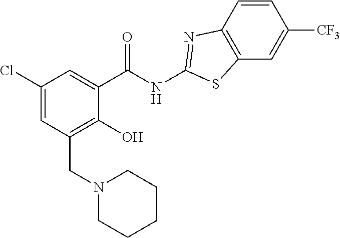 [SMILES: O=C(Nc1nc2ccc(C(F)(F)F)cc2s1)c1cc(Cl)cc(CN2CCCCC2)c1O] | 5-chloro-2-hydroxy-3-(piperidin-1-ylmethyl)- N-(6-(trifluoromethyl)benzo[d]thiazol-2- yl)benzamide |
| 27 | 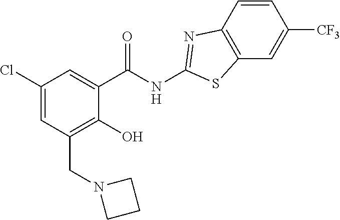 [SMILES: O=C(Nc1nc2ccc(C(F)(F)F)cc2s1)c1cc(Cl)cc(CN2CCC2)c1O] | 3-(azetidin-1-ylmethyl)-5-chloro-2-hydroxy-N- (6-(trifluoromethyl)benzo[d]thiazol-2- yl)benzamide |
| 28 | 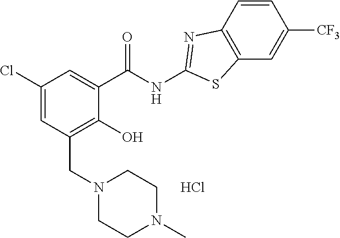 [SMILES: CN1CCN(Cc2cc(Cl)cc(C(=O)Nc3nc4ccc(C(F)(F)F)cc4s3)c2O)CC1 ; Cl] | 5-chloro-2-hydroxy-3-((4-methylpiperazin-1- yl)methyl)-N-(6- (trifluoromethyl)benzo[d]thiazol-2- yl)benzamide |
| 32 | 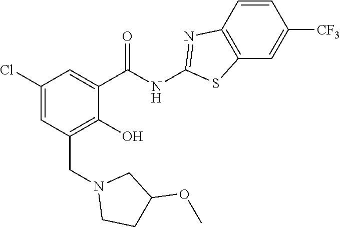 [SMILES: COC1CCN(Cc2cc(Cl)cc(C(=O)Nc3nc4ccc(C(F)(F)F)cc4s3)c2O)C1] | 5-chloro-2-hydroxy-3-((3-methoxypyrrolidin- 1-yl)methyl)-N-(6- (trifluoromethyl)benzo[d]thiazol-2- yl)benzamide |
| 33 | 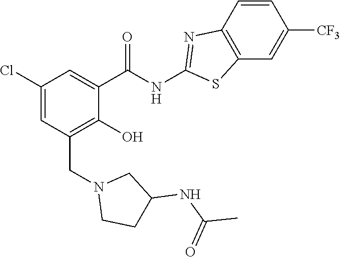 [SMILES: CC(=O)NC1CCN(Cc2cc(Cl)cc(C(=O)Nc3nc4ccc(C(F)(F)F)cc4s3)c2O)C1] | 3-((3-acetamidopyrrolidin-1-yl)methyl)-5- chloro-2-hydroxy-N-(6- (trifluoromethyl)benzo[d]thiazol-2- yl)benzamide |
| 58 | 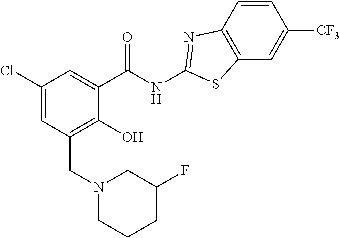 [SMILES: O=C(Nc1nc2ccc(C(F)(F)F)cc2s1)c1cc(Cl)cc(CN2CCCC(F)C2)c1O] | 5-chloro-3-((3-fluoropiperidin-1-yl)methyl)-2- hydroxy-N-(6- (trifluoromethyl)benzo[d]thiazol-2- yl)benzamide |
| 59 | 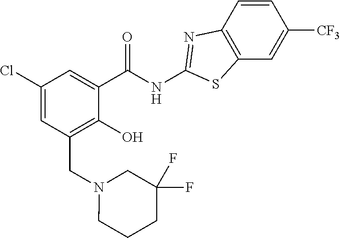 [SMILES: O=C(Nc1nc2ccc(C(F)(F)F)cc2s1)c1cc(Cl)cc(CN2CCCC(F)(F)C2)c1O] | 5-chloro-3-((3,3-difluoropiperidin-1- yl)methyl)-2-hydroxy-N-(6- (trifluoromethyl)benzo[d]thiazol-2- yl)benzamide |
| 60 | 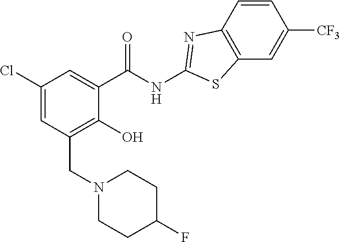 [SMILES: O=C(Nc1nc2ccc(C(F)(F)F)cc2s1)c1cc(Cl)cc(CN2CCC(F)CC2)c1O] | 5-chloro-3-((4-fluoropiperidin-1-yl)methyl)-2- hydroxy-N-(6- (trifluoromethyl)benzo[d]thiazol-2- yl)benzamide |
| 62 | 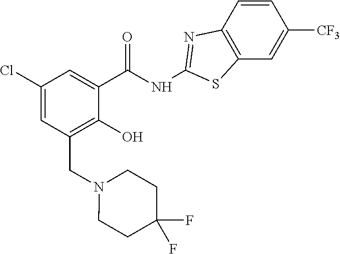 [SMILES: O=C(Nc1nc2ccc(C(F)(F)F)cc2s1)c1cc(Cl)cc(CN2CCC(F)(F)CC2)c1O] | 5-chloro-3-((4,4-difluoropiperidin-1- yl)methyl)-2-hydroxy-N-(6- (trifluoromethyl)benzo[d]thiazol-2- yl)benzamide |

or a pharmaceutically acceptable salt, solvate, or prodrug thereof.

One embodiment describes a compound that is

| | | |
|---|---|---|
| 25 | 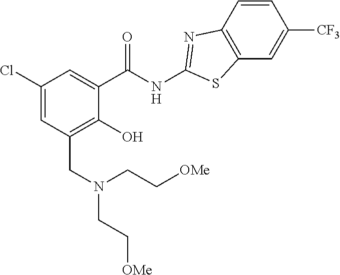 [SMILES: COCCN(CCOC)Cc1cc(Cl)cc(C(=O)Nc2nc3ccc(C(F)(F)F)cc3s2)c1O] | 3-((bis(2-methoxyethyl)amino)methyl)-5- chloro-2-hydroxy-N-(6- (trifluoromethyl)benzo[d]thiazol-2- yl)benzamide(25) 3-((bis(2-methoxyethyl)amino)methyl)-5- chloro-2-hydroxy-N-(6- (trifluoromethyl)benzo[d]thiazol-2- yl)benzamide hydrochloride (25A) |

or a pharmaceutically acceptable salt, solvate, or pro rug thereof.

In one embodiment, the present disclosure describes a compound of Formula III:

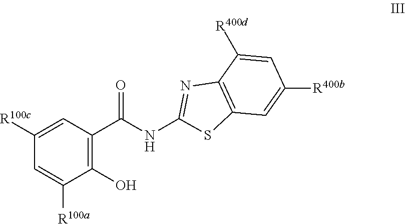

or a pharmaceutically acceptable salt, solvate, or prodrug thereof.

The substituent R^{100a} of Formula III is selected from the group consisting of

—CH₃, —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)NHCO₂CH₃,

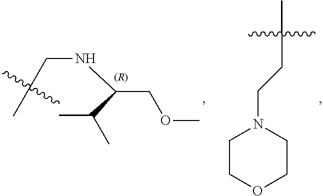

—(CH₂)_{p′}NR^{500A}R^{500B}, —(CH₂)ₚR⁶⁰⁰, and C(O)N(CH₂CH₂OCH₃)₂. Each of substituents R^{5A} and R^{5B}, is independently selected from the group consisting of —C₁-C₆ alkyl; and —C₁-C₆ alkyl substituted with one or more groups selected from —C₁-C₆ alkoxy, and —O(CH₂)₂OCH₃. Alternatively R^{500A} and R^{500B} together with the nitrogen to which they are attached, form a 4 to 8-membered heterocyclyl optionally substituted with one or more substituents, independently selected from the group consisting of oxo, cyano, hydroxyl, alkoxy, acylamino, carboxyamido,
—SO₂CH₃, —CF₃, C₁-C₆ alkyl, halo, and acyl. Integer p′ is an integer selected the group consisting of 1, 2 and 3. Substituent R⁶⁰⁰ is selected from the group consisting of 5 to 6-membered heterocyclyl, pyridinyl and thiazolyl; and p is an integer selected from the group consisting of 0, 1, 2, and 3.

Substituent R^{100c} of Formula III is selected from the group consisting of chloro, fluoro, iodo, and bromo.

Each of substituents R^{400b} and R^{400d} of Formula III is independently selected from the group consisting of Y¹ and Z¹, provided that when R^{400b} is Y¹, R^{400d} is Z¹ and when R^{400b} is Z¹, R^{400d} is Y¹. Substituent Y¹ is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl. Substituent Z¹ is selected from the group consisting of

—CH₂NHC(O)CH₂CH₃, —CH₂NHC(O)CH₂OCH₃, —CH₂NHSO₂CH₃, —(CH₂)_{q′}NR^{7A}R^{7B}, and —(CH₂)_{q}R⁸. Each of R^{7A} and R^{7B} is independently selected from C₁-C₆ alkyl. Alternatively R^{7A} and R^{7B} together with the nitrogen to which they are attached, form a 4 to 8-membered heterocyclyl optionally substituted with one or more independently selected C₁-C₆ alkyl. Integer q′ is an integer selected from the group consisting of 1, 2, and 3. Substituent R⁸ is selected from the group consisting of a 5 to 6-membered heterocyclic ring optionally substituted with methyl, and q′ is an integer selected from the group consisting of 0, 1, 2 and 3.

Further embodiments describe the compound according to Formula IIIa:

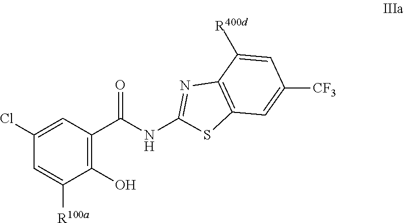

or a pharmaceutically acceptable salt, solvate, or prodrug thereof, wherein each of R^{100a}, and R^{400d} of Formula IIIa is as described previously for Formula III.

In some embodiments, R^{100a} of Formula III, or of Formula IIIa or a pharmaceutically acceptable salt, solvate, or prodrug thereof, is selected from —CH₂NR^{500A}R^{500B}; wherein each of R^{500A} and R^{500B} is independently selected from is independently selected from methyl; and C₁-C₆ alkyl substituted with one or more groups selected from methoxy, and

—O(CH₂)₂OCH₃.

In some embodiments, R^{100a} of Formula III, or of Formula IIIa or a pharmaceutically acceptable salt, solvate, or prodrug thereof, is selected from the group consisting of —CH₂NR^{500A}R^{500B}; wherein each of R^{500A} and R^{500B} is independently selected from C₁-C₆ alkyl substituted with one or more methoxy.

In some embodiments, R^{100a} of Formula III, or of Formula IIIa or a pharmaceutically acceptable salt, solvate, or prodrug thereof, is selected from the group consisting of —CH₂NR^{500A}R^{500B}; wherein R^{500A} and R^{500B} together with the nitrogen to which they are attached, form a 4 to 8-membered heterocyclyl optionally substituted with one or more substituents, independently selected from the group consisting of oxo, cyano, hydroxyl, alkoxy,

-acylamino, carboxyamido, —SO₂CH₃, —CF₃, C₁-C₆ alkyl, halo, and acyl. In some embodiments the 4-8-membered heterocyclyl is selected from the group consisting of azetindinyl, pyrrolidinyl, piperidinyl, piperazinyl, oxopiperazinyl, morpholinyl, dioxothiomorphylinyl, and azabicyclo[3.2.1]octanyl; wherein the heterocyclyl is optionally substituted with one or more substituents, independently selected from the group consisting of oxo, cyano, hydroxyl, alkoxy,
-acylamino, carboxyamido, —SO₂CH₃, —CF₃, C₁-C₆ alkyl, halo, and acyl. In some embodiments the 4-8-membered heterocyclyl is optionally substituted with one or more substituents independently selected from the group consisting of oxo, cyano, hydroxyl, methoxy,
—NHC(O)CH₃, —C(O)NH₂, —SO₂CH₃, —CF₃, methyl, fluoro, and acetyl. In some embodiments the 4-8-membered heterocyclyl is optionally substituted with one or more methyl substituents. In some embodiments the 4 to 8-membered heterocyclyl is selected from the group consisting of azetindinyl; pyrrolidinyl; pyrrolidinyl substituted with one or more substituents selected from the group consisting of cyano, hydroxyl, methoxy, —NHC(O)CH₃, —C(O)NH₂, —SO₂CH₃, —CF₃ and methyl; piperidinyl; piperidinyl substituted with one or more substituents selected from the group consisting of —CF₃, and fluoro; piperazinyl substituted with one or more substituents selected from the group consisting of oxo, methyl, and acetyl; morpholinyl; morpholinyl substituted with one or more methyl groups;
dioxothiomorphylinyl, and azabicyclo[3.2.1]octanyl.

In some embodiments, R^{100a} of Formula III, or of Formula IIIa or a pharmaceutically acceptable salt, solvate, or prodrug thereof, is selected from the group consisting of —CH₂NR^{500A}R^{500B}; wherein R^{500A} and R^{500B} together with the nitrogen to which they are attached, form a 6-membered heterocyclyl optionally substituted with one or more substituents, independently selected from the group consisting of oxo, cyano, hydroxyl, alkoxy,

-acylamino, carboxyamido, —SO₂CH₃, —CF₃, C₁-C₆ alkyl, halo, and acyl. In some embodiments the 6-membered heterocyclyl is selected from the group consisting piperazinyl optionally substituted with one or C₁-C₆ alkyl substituents, and morpholinyl optionally substituted with one or C₁-C₆ alkyl substituents.

In some embodiments, R^{100a} of Formula III, or of Formula IIIa or a pharmaceutically acceptable salt, solvate, or prodrug thereof, is selected from the group consisting of —CH₂NR^{500A}R^{500B}; wherein —CH₂NR^{500A}R^{500B} is selected from the group consisting of —CH₂N(CH₃)(CH₂)₂OCH₃, —(CH₂)N(CH₂CH₂OCH₃)₂, —CH₂N(CH₃)(CH₂)₂O(CH₂)₂OCH₃,

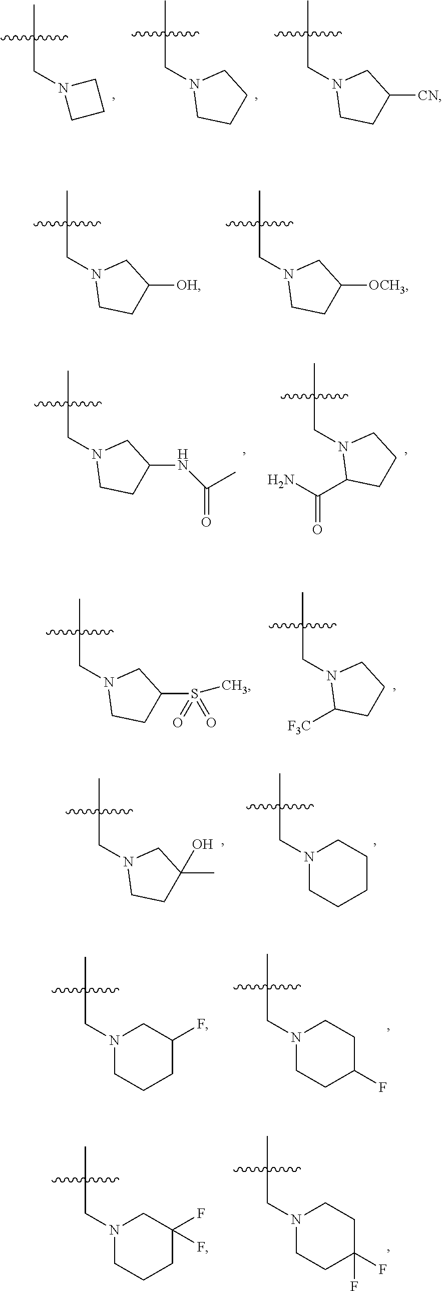

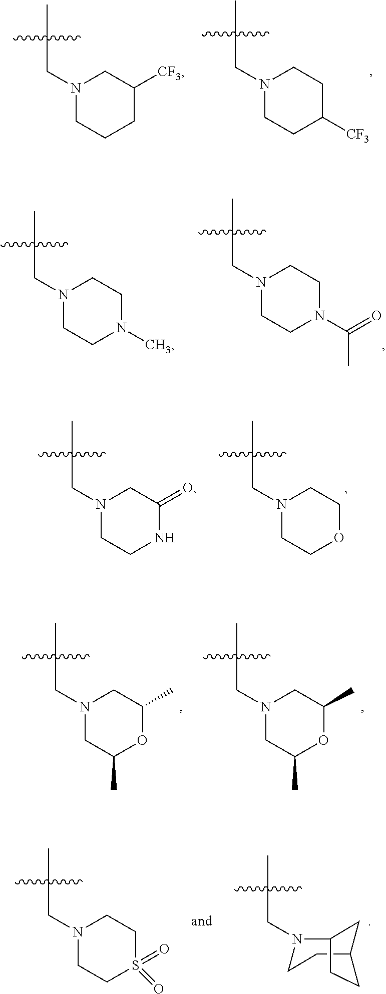

In some embodiments —CH₂NR^{500A}R^{500B} is selected from the group consisting of
—(CH₂)N(CH₂CH₂OCH₃)₂,

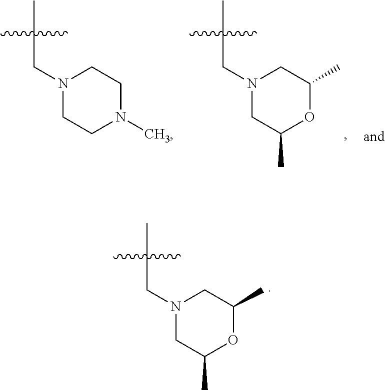

In some embodiments, R^{100a} of Formula III, or of Formula IIIa or a pharmaceutically acceptable salt, solvate, or prodrug thereof, is —(CH₂)ₚR⁶⁰⁰; wherein R⁶⁰⁰ is selected from the group consisting of tetrahydrofuranyl, tetrahydropyranyl, piperidinyl, thiazolyl and piperidinyl and m is as previously described. In some embodiments p is 0. In some embodiments p is 1. In some embodiments —(CH₂)ₚR⁶⁰⁰ is selected from the group consisting of

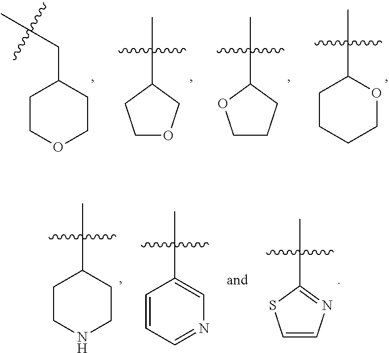

In some embodiments, R^{100a} of Formula III, or of Formula IIIa or a pharmaceutically acceptable salt, solvate, or prodrug thereof, is selected from the group consisting of —CH₃, —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, —(CH₂)₂NHCO₂CH₃,

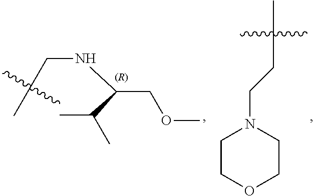

—CH₂N(CH₃)(CH₂)₂OCH₃, —(CH₂)N(CH₂CH₂OCH₃)₂, —CH₂N(CH₃)(CH₂)₂O(CH₂)₂OCH₃,

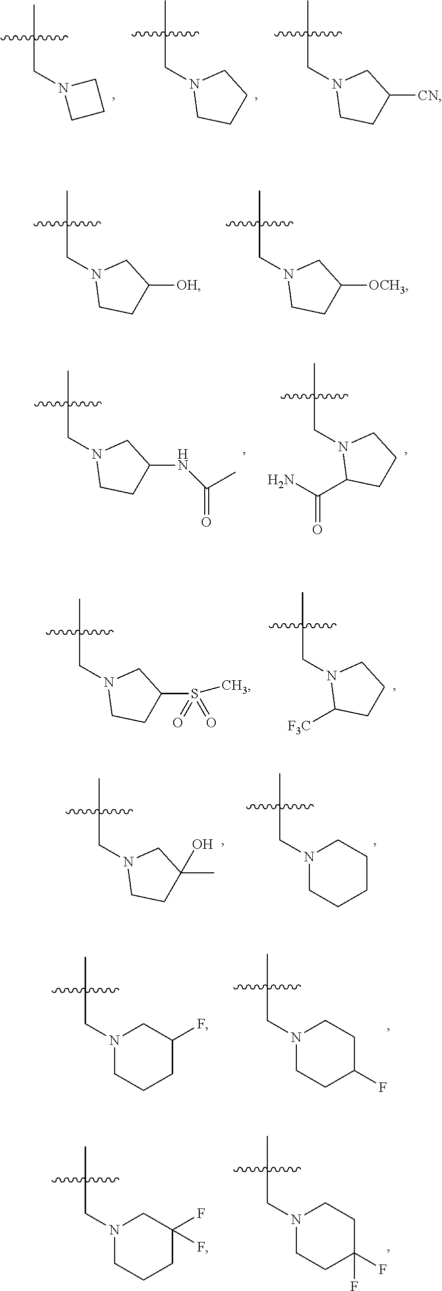

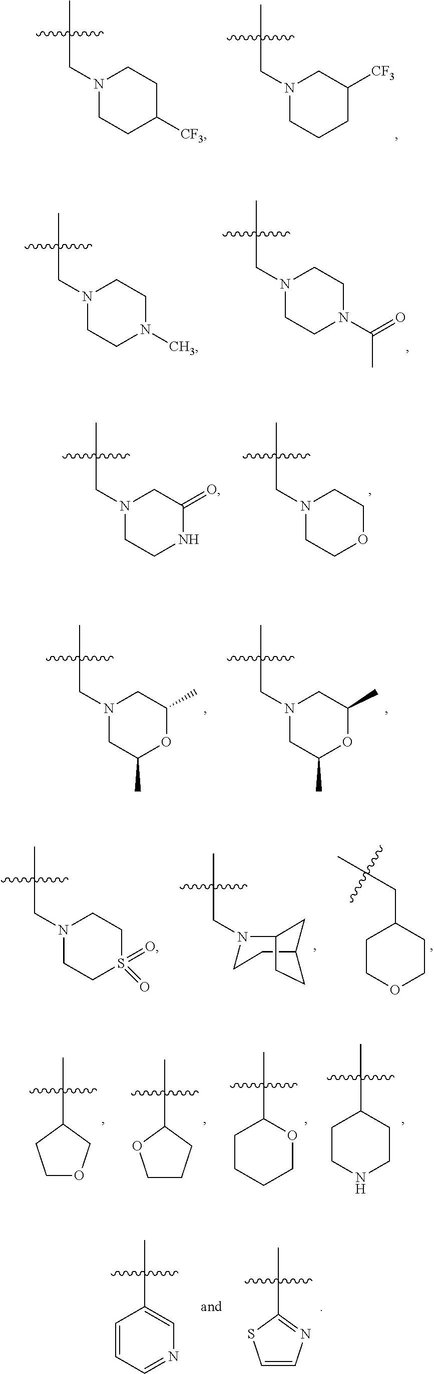

In some embodiments, R^{100a} of Formula III, or of Formula IIIa or a pharmaceutically acceptable salt, solvate, or prodrug thereof, is selected from the group consisting of —CH₃, and —CH₂NR^{500A}R^{500B} wherein R^{500A} and R^{500B} are as previously described in any embodiment described herein. In some embodiments, each of R^{500A} and R^{500B} is independently selected from C₁-C₆ alkyl substituted with one or more methoxy; alternatively, R^{500A} and R^{500B} together with the nitrogen to which they are attached, form a 6-membered heterocyclyl optionally substituted with one or more substituents, independently selected from C₁-C₆ alkyl.

In some embodiments, R^{10a} of Formula III, or of Formula IIIa or a pharmaceutically acceptable salt, solvate, or prodrug thereof, is selected from the group consisting of —CH₃, —(CH₂)N(CH₂CH₂OCH₃)₂

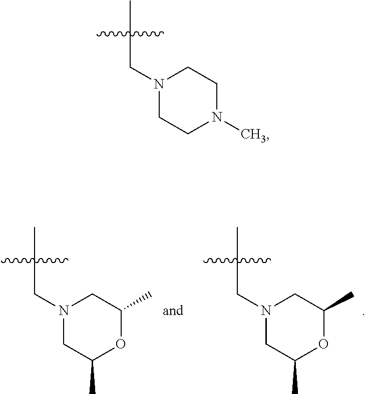

In some embodiments, R^{100c} of Formula III, or a pharmaceutically acceptable salt, solvate, or prodrug thereof, is chloro.

Some embodiments describe a compound of Formula III, or a pharmaceutically acceptable salt, solvate, or prodrug thereof, wherein R^{400b} is Y¹ and R^{400d} is Z¹.

Some embodiments describe a compound of Formula III, or a pharmaceutically acceptable salt, solvate, or prodrug thereof, wherein R^{400b} is Y¹ and R^{400d} is Z¹.

In some embodiments, Y¹ of Formula III, or a pharmaceutically acceptable salt, solvate, or prodrug thereof, is —CF₃.

In some embodiments, Z¹ of Formula III, or R^{400d} of Formula IIIa, or a pharmaceutically acceptable salt, solvate, or prodrug thereof, is —CH₂NR^{7A}R^{7B} wherein R^{7A} and R^{7B} are as previously described herein. In some embodiments each of R^{7A} and R^{7B} is methyl. In some embodiments R^{7A} and R^{7B} together with the nitrogen to which they are attached, form a

6-membered heterocyclyl optionally substituted with a methyl group. In some embodiments R^{7A} and R^{7B} together with the nitrogen to which they are attached, form a heterocyclyl selected from the group consisting of piperazinyl optionally substituted with one or more C₁-C₆ alkyl and morpholinyl optionally substituted with one or more C₁-C₆ alkyl. In some embodiments R^{7A} and R^{7B} together with the nitrogen to which they are attached, form a heterocyclyl selected from the group consisting of, 4-methylpiperazinyl, and morpholinyl. In some embodiments
—CH₂NR^{7A}R^{7B} is selected from the group consisting of —CH₂N(CH₃)₂,

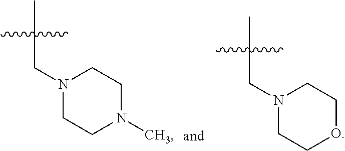 [SMILES: *CN1CCN(C)CC1 ; *CN1CCOCC1]

In some embodiments, Z¹ of Formula III, or R^{400d} of Formula IIIa, or a pharmaceutically acceptable salt, solvate, or prodrug thereof, is —(CH₂)_{q}R⁸, wherein R⁸ is as previously described herein. In some embodiments R⁸ is a 6-membered heterocyclic ring optionally substituted with methyl. In some embodiments R⁸ is 4-methylpiperidinyl. In some embodiments q is 0. In some embodiments q is 1. In some embodiments —(CH₂)_{q}R⁸ is

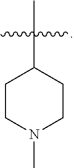 [SMILES: *C1CCN(C)CC1]

In some embodiments, Z¹ of Formula I, or R^{400d} of Formula IIIa, or a pharmaceutically acceptable salt, solvate, or prodrug thereof, is selected from the group consisting of —CH₂NHC(O)CH₂CH₃, —CH₂NHC(O)CH₂OCH₃, —CH₂NHSO₂CH₃, —CH₂NR^{7A}R^{7B}, and —(CH₂)_{q}R⁸, wherein R^{7A}, R^{7B}, R⁸, and p are as described in any embodiment herein.

In some embodiments, Z¹ of Formula III, or R^{400d} of Formula IIIa, or a pharmaceutically acceptable salt, solvate, or prodrug thereof, is selected from the group consisting of —CH₂NHC(O)CH₂CH₃, —CH₂NHC(O)CH₂OCH₃, —CH₂NHSO₂CH₃, —CH₂NR^{7A}R^{7B}, and —(CH₂)_{q}R⁸ wherein R^{7A}, R^{7B}, R⁸, and p are as described in any embodiment herein.

In some embodiments, Z¹ of Formula III, or R^{400d} of Formula IIIa, or a pharmaceutically acceptable salt, solvate, or prodrug thereof, is selected from the group consisting of —CH₂NHC(O)CH₂CH₃, —CH₂NHC(O)CH₂OCH₃, —CH₂NHSO₂CH₃, —CH₂NR^{7A}R^{7B}, and —(CH₂)_{q}R⁸, wherein each of R^{7A} and R^{7B} is methyl. Alternatively embodiments R^{7A} and R^{7B} together with the nitrogen to which they are attached, form a heterocyclyl selected from the group consisting of, 4-methylpiperazinyl, and morpholinyl; and R⁸ is 4-methylpiperidinyl.

In some embodiments, Z¹ of Formula III, or R^{400d} of Formula IIIa, or a pharmaceutically acceptable salt, solvate, or prodrug thereof, is selected from the group consisting of —CH₂NHC(O)CH₂CH₃, —CH₂NHC(O)CH₂OCH₃, —CH₂NHSO₂CH₃, —CH₂N(CH₃)₂,

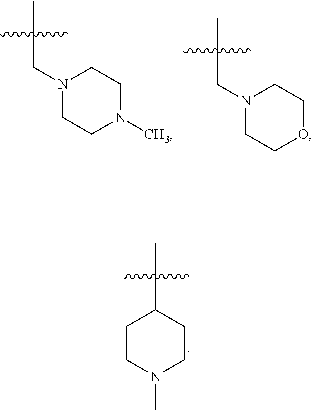

In some embodiments, Z¹ of Formula III, or R^{400d} of Formula IIIa, or a pharmaceutically acceptable salt, solvate, or prodrug thereof, is selected from the group consisting of —CH₂NHC(O)CH₂CH₃, —CH₂NHC(O)CH₂OCH₃, —CH₂NHSO₂CH₃, —CH₂N(CH₃)₂,

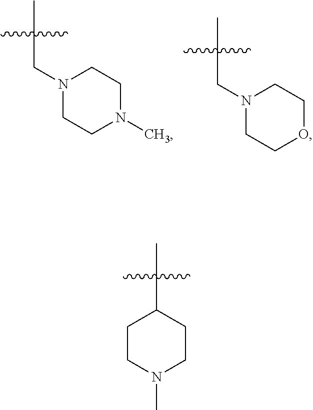

and Y¹ of Formula III is selected from the group consisting of chloro, fluoro, iodo, bromo, —CF₃, —CHF₂, fluoro(C₁-C₆)alkyl, halo(C₁-C₆)alkyl, —OCF₃,
—SO₂(C₁-C₆)alkyl, cyano, and —CO₂(C₁-C₆)alkyl.

In some embodiments, Z¹ of Formula III, or R^{400d} of Formula IIIa, or a pharmaceutically acceptable salt, solvate, or prodrug thereof, is selected from the group consisting of —CH₂NHC(O)CH₂CH₃, —CH₂NHC(O)CH₂OCH₃, —CH₂NHSO₂CH₃, —CH₂N(CH₃)₂,

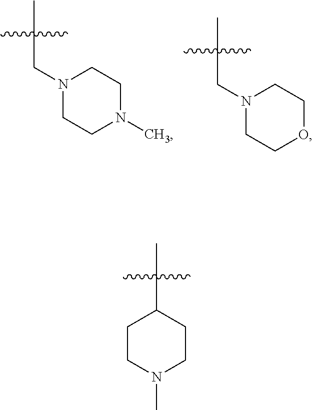

and Y¹ of Formula III is —CF₃.

In some embodiments, R^{400d} of Formula III, or a pharmaceutically acceptable salt, solvate, or prodrug thereof, is selected from the group consisting of —CH₂NHC(O)CH₂CH₃, —CH₂NHC(O)CH₂OCH₃, —CH₂NHSO₂CH₃, —CH₂N(CH₃)₂,

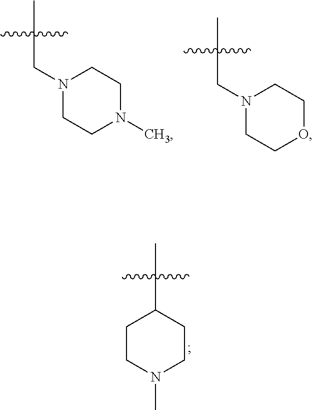

and R^{400b} of Formula III is —CF₃.

Some embodiments describe a compound of Formula III or a pharmaceutically acceptable salt, solvate, or prodrug thereof

wherein:
R^{100a} is selected from the group consisting of —CH₃, and —CH₂NR^{500A}R^{500B},
each of R^{500A} and R^{500B} is independently selected from C₁-C₆ alkyl substituted with one or more methoxy; alternatively, R^{500A} and R^{500B} together with the nitrogen to which they are attached, form a 6-membered heterocyclyl optionally substituted with one or more substituents, independently selected from C₁-C₆ alkyl;
R^{100c} is chloro;
each of R^{400b} and R^{400d} is independently selected from the group consisting of Y¹ and Z¹, provided that when R^{400b} is Y¹, R^{400d} is Z¹ and when R^{400b} is Z¹, R^{400d} is Y¹.
Y¹ is selected from the group consisting of chloro, fluoro, iodo, bromo, —CF₃, —CHF₂, fluoro(C₁-C₆)alkyl, halo(C₁-C₆)alkyl, —OCF₃, —SO₂(C₁-C₆)alkyl, cyano, and —CO₂(C₁-C₆)alkyl;
Z¹ is selected from the group consisting of —CH₂NHC(O)CH₂OCH₃, —CH₂NHC(O)CH₂CH₃, —CH₂NHSO₂CH₃, —CH₂NR^{7A}R^{7B}, and —(CH₂)_{q}R⁸, wherein each of R^{7A} and R^{7B} is methyl; alternatively R^{7A} and R^{7B} together with the nitrogen to which they are attached, form a heterocyclyl selected from the group consisting of, 4-methylpiperazinyl, and morpholinyl; and R⁸ is 4-methylpiperidinyl.

Some embodiments describe a compound of Formula III or a pharmaceutically acceptable salt, solvate, or prodrug thereof

wherein:
R^{100a} is selected from the group consisting of —CH₃, and —CH₂NR^{500A}R^{500B},
each of R^{500A} and R^{500B} is independently selected from C₁-C₆ alkyl substituted with one or more methoxy; alternatively, R^{500A} and R^{500B} together with the nitrogen to which they are attached, form a 6-membered heterocyclyl optionally substituted with one or more substituents, independently selected from C₁-C₆ alkyl;
R^{100c} is chloro;
each of R^{400b} and R^{400d} is independently selected from the group consisting of Y¹ and Z¹, provided that when R^{400b} is Y¹, R^{400d} is Z¹ and when R^{400d} is Z¹, R^{400b} is Y¹.
Y¹ is —CF₃;
Z¹ is selected from the group consisting of —CH₂NHC(O)CH₂OCH₃, —CH₂NHC(O)CH₂CH₃, —CH₂NHSO₂CH₃, —CH₂NR^{7A}R^{7B}, and —(CH₂)_{q}R⁸, wherein each of R^{7A} and R^{7B} is methyl; alternatively R^{7A} and R^{7B} together with the nitrogen to which they are attached, form a heterocyclyl selected from the group consisting of, 4-methylpiperazinyl, and morpholinyl; and R⁸ is 4-methylpiperidinyl

Some embodiments describe a compound of Formula III or a pharmaceutically acceptable salt, solvate, or prodrug thereof

wherein:
R^{100a} is selected from the group consisting of —CH₃, —(CH₂)N(CH₂CH₂OCH₃)₂,

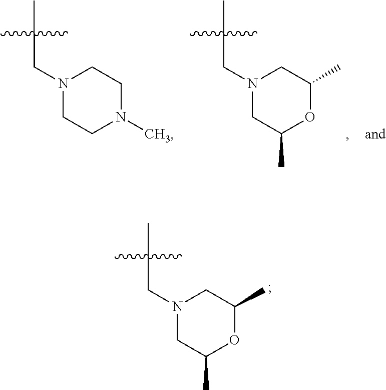

R^{100c} is chloro;
each of R^{400b} and R^{400d} is independently selected from the group consisting of Y¹ and Z¹, provided that when R^{400b} is Y¹, R^{400d} is Z¹ and when R^{400b} is Z¹, R^{400d} is Y¹; Y¹ is CF₃; and Z¹ is selected from the group consisting of —CH₂NHC(O)CH₂OCH₃, —CH₂NHC(O)CH₂CH₃,
   —CH₂NHSO₂CH₃, —CH₂N(CH₃)₂,

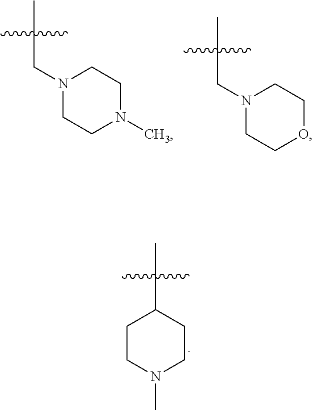

Some embodiments describe a compound of Formula III or a pharmaceutically acceptable salt, solvate, or prodrug thereof

wherein:
R^{100a} is selected from the group consisting of —CH₃, —(CH₂)N(CH₂CH₂OCH₃)₂,

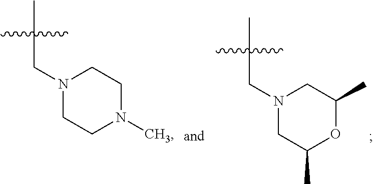

R^{100c} is chloro;
R^{400b} is CF₃; and
R^{400d} is selected from the group consisting of —CH₂NHC(O)CH₂OCH₃, —CH₂NHC(O)CH₂CH₃, —CH₂NHSO₂CH₃, —CH₂N(CH₃)₂,

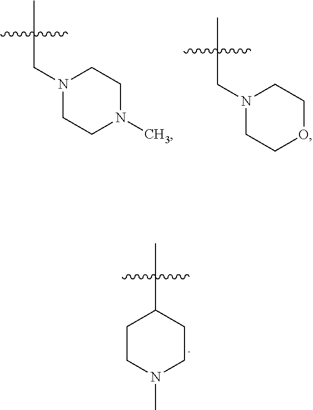

Some embodiments describe a compound of Formula IIIa or a pharmaceutically acceptable salt, solvate, or prodrug thereof

wherein:
R^{100a} is selected from the group consisting of —CH₃, —(CH₂)N(CH₂CH₂OCH₃)₂,

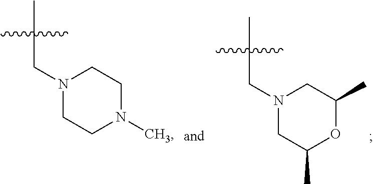

and
R^{400d} is selected from the group consisting of —CH₂NHC(O)CH₂CH₃, —CH₂NHC(O)CH₂OCH₃, —CH₂NHSO₂CH₃, —CH₂N(CH₃)₂,

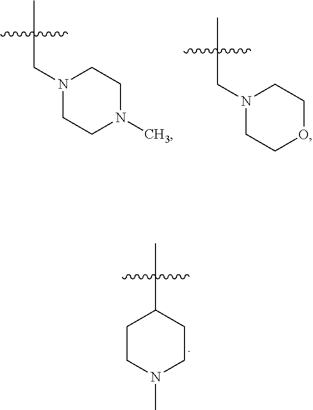

Some embodiments describe a compound of Formula IIIa or a pharmaceutically acceptable salt, solvate, or prodrug thereof

wherein:

Some embodiments describe a compound selected from the group consisting of:

| Number | Structure | Name |
|---|---|---|
| 72 | 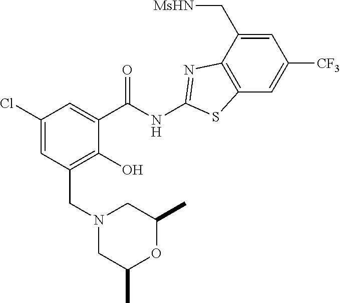 | 5-chloro-3-((cis-2,6- dimethylmorpholino)methyl)-2-hydroxy-N-(4- (methylsulfonamidomethyl)-6- (trifluoromethyl)benzo[d]thiazol-2- yl)benzamide |
| 73 | 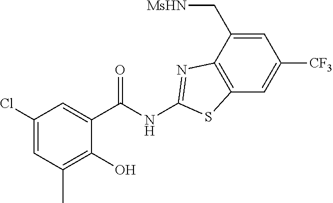 | 5-chloro-2-hydroxy-3-methyl-N-(4- (methylsulfonamidomethyl)-6- (trifluoromethyl)benzo[d]thiazol-2- yl)benzamide |
| 74 | 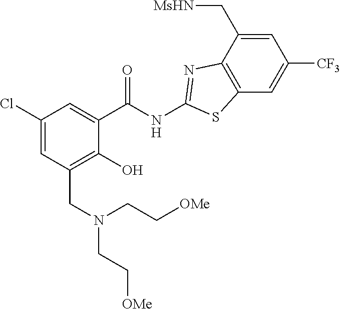 | 3-((bis(2-methoxyethyl)amino)methyl)-5- chloro-2-hydroxy-N-(4- (methylsulfonamidomethyl)-6- (trifluoromethyl)benzo[d]thiazol-2- yl)benzamid |
| 78 | 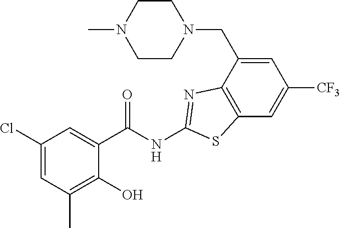 [SMILES: Cc1cc(Cl)cc(C(=O)Nc2nc3c(CN4CCN(C)CC4)cc(C(F)(F)F)cc3s2)c1O] | 5-chloro-2-hydroxy-3-methyl-N-(4-((4- methylpiperazin-1-yl)methyl)-6- (trifluoromethyl)benzo[d]thiazol-2- yl)benzamide |
| 79 | 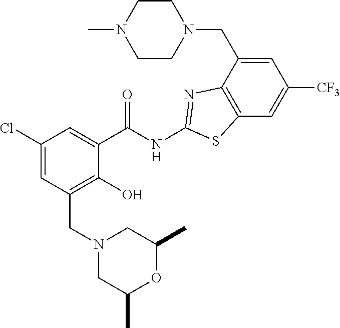 [SMILES: C[C@@H]1CN(Cc2cc(Cl)cc(C(=O)Nc3nc4c(CN5CCN(C)CC5)cc(C(F)(F)F)cc4s3)c2O)C[C@H](C)O1] | 5-chloro-3-((cis-2,6- dimethylmorpholino)methyl)-2-hydroxy-N-(4- ((4-methylpiperazin-1-yl)methyl)-6- (trifluoromethyl)benzo[d]thiazol-2- yl)benzamide |
| 80 | 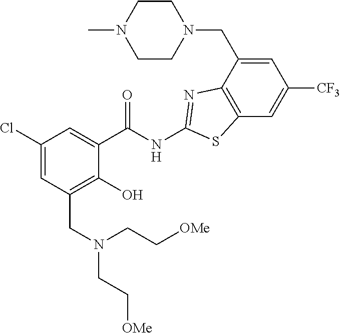 [SMILES: COCCN(CCOC)Cc1cc(Cl)cc(C(=O)Nc2nc3c(CN4CCN(C)CC4)cc(C(F)(F)F)cc3s2)c1O] | 3-((bis(2-methoxyethyl)amino)methyl)-5- chloro-2-hydroxy-N-(4-((4-methylpiperazin-1- yl)methyl)-6-(trifluoromethyl)benzo[d]thiazol- 2-yl)benzamide |
| 81 | 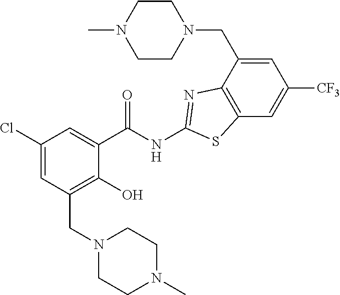 [SMILES: CN1CCN(Cc2cc(Cl)cc(C(=O)Nc3nc4c(CN5CCN(C)CC5)cc(C(F)(F)F)cc4s3)c2O)CC1] | 5-chloro-2-hydroxy-3-((4-methylpiperazin-1- yl)methyl)-N-(4-((4-methylpiperazin-1- yl)methyl)-6-(trifluoromethyl)benzo[d]thiazol- 2-yl)benzamide |
| 90 | 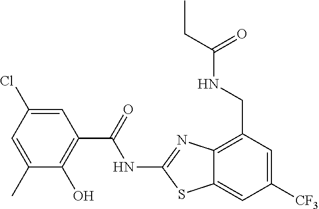 [SMILES: CCC(=O)NCc1cc(C(F)(F)F)cc2sc(NC(=O)c3cc(Cl)cc(C)c3O)nc12] | 5-chloro-2-hydroxy-3-methyl-N-(4- (propionamidomethyl)-6- (trifluoromethyl)benzo[d]thiazol-2- yl)benzamide |
| 94 | 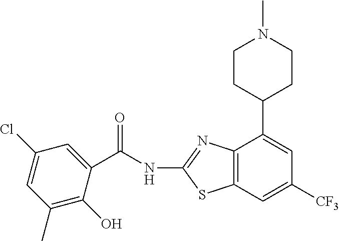 [SMILES: Cc1cc(Cl)cc(C(=O)Nc2nc3c(C4CCN(C)CC4)cc(C(F)(F)F)cc3s2)c1O] | 5-chloro-2-hydroxy-3-methyl-N-(4-(1- methylpiperidin-4-yl)-6- (trifluoromethyl)benzo[d]thiazol-2- yl)benzamide |
| 95 | 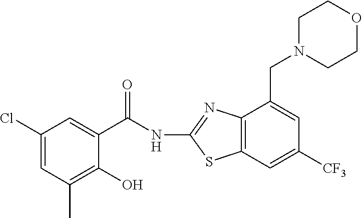 [SMILES: Cc1cc(Cl)cc(C(=O)Nc2nc3c(CN4CCOCC4)cc(C(F)(F)F)cc3s2)c1O] | 5-chloro-2-hydroxy-3-methyl-N-(4- (morpholinomethyl)-6-(trifluoro- methyl)benzo[d]thiazol-2-yl)benzamide |
| 96 | 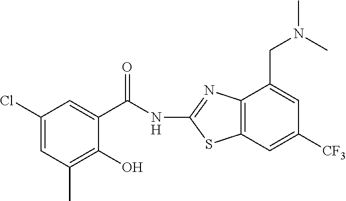 [SMILES: Cc1cc(Cl)cc(C(=O)Nc2nc3c(CN(C)C)cc(C(F)(F)F)cc3s2)c1O] | 5-chloro-N-(4-((dimethylamino)methyl)-6- (trifluoromethyl)benzo[d]thiazol-2-yl)-2- hydroxy-3-methylbenzamide |
| 101 | 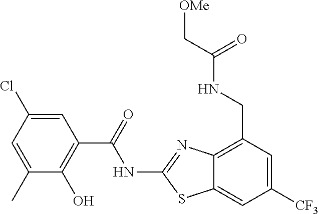 [SMILES: COCC(=O)NCc1cc(C(F)(F)F)cc2sc(NC(=O)c3cc(Cl)cc(C)c3O)nc12] | 5-chloro-2-hydroxy-N-(4-((2- methoxyacetamido)methyl)-6- (trifluoromethyl)benzo[d]thiazol-2-yl)-3- methylbenzamide |

or a pharmaceutically acceptable salt, solvate, or prodrug thereof.

Some embodiments describe a compound selected from the group consisting of:

| Number | Structure | Name |
|---|---|---|
| 72 | 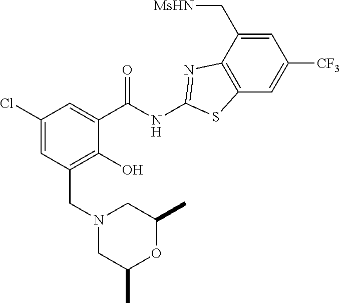 | 5-chloro-3-((cis-2,6- dimethylmorpholino)methyl)-2-hydroxy-N-(4- (methylsulfonamidomethyl)-6- (trifluoromethyl)benzo[d]thiazol-2- yl)benzamide |
| 74 | 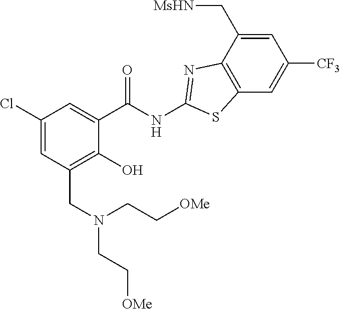 | 3-((bis(2-methoxyethyl)amino)methyl)-5- chloro-2-hydroxy-N-(4- (methylsulfonamidomethyl)-6- (trifluoromethyl)benzo[d]thiazol-2- yl)benzamid |
| 78 | 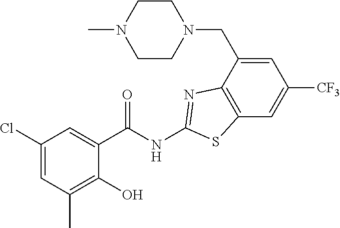 [SMILES: Cc1cc(Cl)cc(C(=O)Nc2nc3c(CN4CCN(C)CC4)cc(C(F)(F)F)cc3s2)c1O] | 5-chloro-2-hydroxy-3-methyl-N-(4-((4- methylpiperazin-1-yl)methyl)-6- (trifluoromethyl)benzo[d]thiazol-2- yl)benzamide |
| 79 | 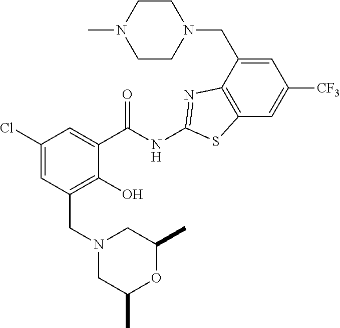 [SMILES: C[C@@H]1CN(Cc2cc(Cl)cc(C(=O)Nc3nc4c(CN5CCN(C)CC5)cc(C(F)(F)F)cc4s3)c2O)C[C@H](C)O1] | 5-chloro-3-((cis-2,6- dimethylmorpholino)methyl)-2-hydroxy-N-(4- ((4-methylpiperazin-1-yl)methyl)-6- (trifluoromethyl)benzo[d]thiazol-2- yl)benzamide |

or a pharmaceutically acceptable salt, solvate, or prodrug thereof.

Additional compounds that are mitochondrial uncouplers but may not necessarily have the unexpected properties of the other compounds disclosed herein include:

| Number | Structure | Name |
|---|---|---|
| 4 | 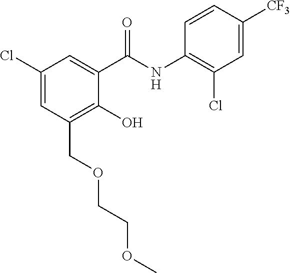 [SMILES: COCCOCc1cc(Cl)cc(C(=O)Nc2ccc(C(F)(F)F)cc2Cl)c1O] | 5-chloro-N-(2-chloro-4- (trifluoromethyl)phenyl)-2-hydroxy-3-((2- methoxyethoxy)methyl)benzamide |
| 6 | 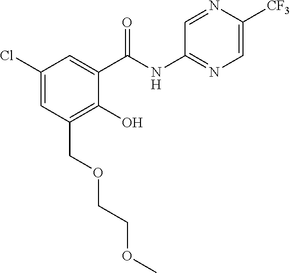 [SMILES: COCCOCc1cc(Cl)cc(C(=O)Nc2cnc(C(F)(F)F)cn2)c1O] | 5-chloro-2-hydroxy-3-((2- methoxyethoxy)methyl)-N-(5- (trifluoromethyl)pyrazin-2-yl)benzamide |
| 18 | 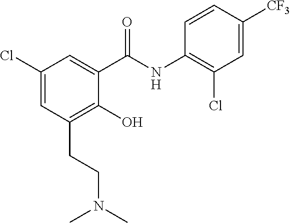 [SMILES: CN(C)CCc1cc(Cl)cc(C(=O)Nc2ccc(C(F)(F)F)cc2Cl)c1O] | 5-chloro-N-(2-chloro-4- (trifluoromethyl)phenyl)-3-(2- (dimethylamino)ethyl)-2-hydroxybenzamide |
| 20 | 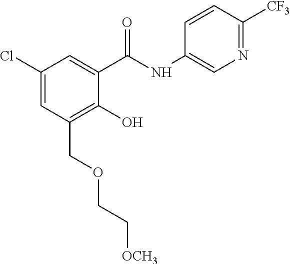 [SMILES: COCCOCc1cc(Cl)cc(C(=O)Nc2ccc(C(F)(F)F)nc2)c1O] | 5-chloro-2-hydroxy-3-((2- methoxyethoxy)methyl)-N-(6- (trifluoromethyl)pyridin-3-yl)benzamide (20) 5-chloro-2-hydroxy-3-((2- methoxyethoxy)methyl)-N-(6- (trifluoromethyl)pyridin-3-yl)benzamide hydrochloride (20A) |
| 21 | 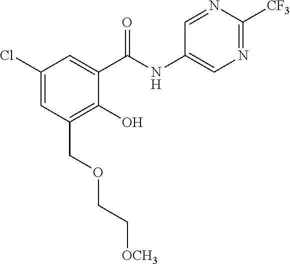 [SMILES: COCCOCc1cc(Cl)cc(C(=O)Nc2cnc(C(F)(F)F)nc2)c1O] | 5-chloro-2-hydroxy-3-((2- methoxyethoxy)methyl)-N-(2- (trifluoromethyl)pyrimidin-5-yl)benzamide |
| 44 | 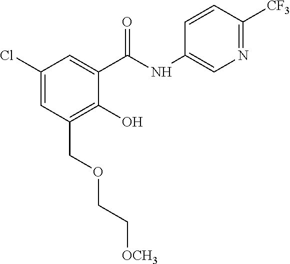 [SMILES: COCCOCc1cc(Cl)cc(C(=O)Nc2ccc(C(F)(F)F)nc2)c1O] | 5-chloro-2-hydroxy-3-((2- methoxyethoxy)methyl)-N-(6- (trifluoromethyl)pyridin-3-yl)benzamide (44) 5-chloro-2-hydroxy-3-((2- methoxyethoxy)methyl)-N-(6- (trifluoromethyl)pyridin-3-yl)benzamide hydrochloride (44A) |
| 45 | 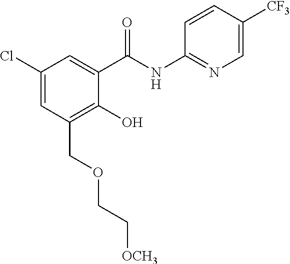 [SMILES: COCCOCc1cc(Cl)cc(C(=O)Nc2ccc(C(F)(F)F)cn2)c1O] | 5-chloro-2-hydroxy-3-((2- methoxyethoxy)methyl)-N-(5- (trifluoromethyl)pyridin-2-yl)benzamide |
| 47 | 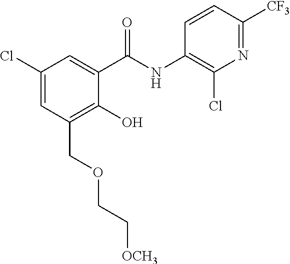 [SMILES: COCCOCc1cc(Cl)cc(C(=O)Nc2ccc(C(F)(F)F)nc2Cl)c1O] | 5-chloro-N-(2-chloro-6- (trifluoromethyl)pyridin-3-yl)-2-hydroxy-3- ((2-methoxyethoxy)methyl)benzamide |
| 50 | 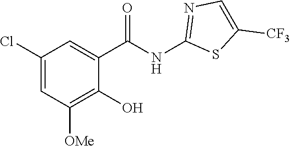 [SMILES: COc1cc(Cl)cc(C(=O)Nc2ncc(C(F)(F)F)s2)c1O] | 5-chloro-2-hydroxy-3-methoxy-N-(5- (trifluoromethyl)thiazol-2-yl)benzamide |
| 51 | 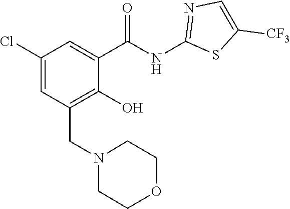 [SMILES: O=C(Nc1ncc(C(F)(F)F)s1)c1cc(Cl)cc(CN2CCOCC2)c1O] | 5-chloro-2-hydroxy-3-(morpholinomethyl)-N- (5-(trifluoromethyl)thiazol-2-yl)benzamide |
| 52 | 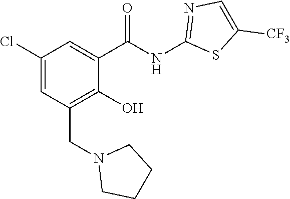 [SMILES: O=C(Nc1ncc(C(F)(F)F)s1)c1cc(Cl)cc(CN2CCCC2)c1O] | 5-chloro-2-hydroxy-3-(pyrrolidin-1-ylmethyl)- N-(5-(trifluoromethyl)thiazol-2-yl)benzamide |
| 53 | 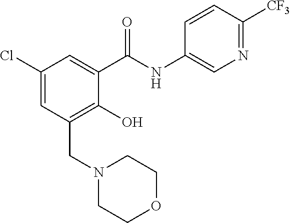 [SMILES: O=C(Nc1ccc(C(F)(F)F)nc1)c1cc(Cl)cc(CN2CCOCC2)c1O] | 5-chloro-2-hydroxy-3-(morpholinomethyl)-N- (6-(trifluoromethyl)pyridin-3-yl)benzamide (53) 5-chloro-2-hydroxy-3-(morpholinomethyl)-N- (6-(trifluoromethyl)pyridin-3-yl)benzamide hydrochloride (53A) |
| 54 | 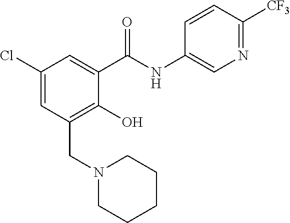 [SMILES: O=C(Nc1ccc(C(F)(F)F)nc1)c1cc(Cl)cc(CN2CCCCC2)c1O] | 5-chloro-2-hydroxy-3-(piperidin-1-ylmethyl)- N-(6-(trifluoromethyl)pyridin-3-yl)benzamide (54) 5-chloro-2-hydroxy-3-(piperidin-1-ylmethyl)- N-(6-(trifluoromethyl)pyridin-3-yl)benzamide hydrochloride (54A) |
| 55 | 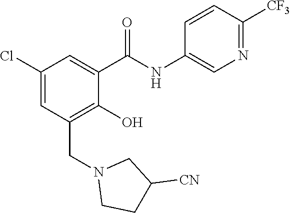 [SMILES: N#CC1CCN(Cc2cc(Cl)cc(C(=O)Nc3ccc(C(F)(F)F)nc3)c2O)C1] | 5-chloro-3-((3-cyanopyrrolidin-1-yl)methyl)-2- hydroxy-N-(6-(trifluoromethyl)pyridin-3- yl)benzamide |
| 98 | 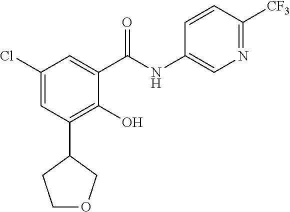 [SMILES: O=C(Nc1ccc(C(F)(F)F)nc1)c1cc(Cl)cc(C2CCOC2)c1O] | 5-chloro-2-hydroxy-3-(tetrahydrofuran-3-yl)- N-(6-(trifluoromethyl)pyridin-3-yl)benzamide |

or a pharmaceutically acceptable salt, solvate, or prodrug thereof.

The compounds of embodiments described herein can possess one or more asymmetric carbon atoms and are thus capable of existing in the form of optical isomers as well as in the form of racemic or non-racemic mixtures thereof. The compounds can be utilized in embodiments described herein as a single isomer or as a mixture of stereochemical isomeric forms. Diastereoisomers, i.e., non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of the invention can likewise be obtained by utilizing optically active starting materials. These isomers may be in the form of a free acid, a free base, an ester or a salt.

Compounds according to embodiments described herein may be in the form of pharmaceutically acceptable salts. A pharmaceutically acceptable salt of the compounds described herein includes acid addition salts and base addition salts. Pharmaceutically-acceptable salt embraces salts commonly used to form alkali metal salts and to form addition salts of free acids or free bases The nature of the salt is not critical, provided that it is pharmaceutically-acceptable. Suitable pharmaceutically-acceptable acid addition salts of the compounds described herein may be prepared from an inorganic acid or an organic acid. Examples of such inorganic acids include, without limitation, hydrochloric, hydrobromic, hydroiodic, nitric, carbonic, sulfuric and phosphoric acid. In some embodiments the salt is a hydrochloride salt. Appropriate organic acids may be selected from aliphatic, cycloaliphatic, aromatic, arylaliphatic, heterocyclic, carboxylic and sulfonic classes of organic acids, examples of which include, without limitation, formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, s-hydroxybutyric, malonic, galactic, and galacturonic acid. Pharmaceutically-acceptable base addition salts for compounds described herein can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl)amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cyclo alkyl) amines, substituted cycloalkyl amines, disubstituted cycloalkyl amine, trisubstituted cycloalkyl amines, cycloalkenyl amines, di(cycloalkenyl) amines, tri(cycloalkenyl)amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl)amine, tri(n-propyl)amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the preparation of pharmaceutically acceptable salts, for example, carboxylic acid amides, including carboxamides, lower alkyl carboxamides, dialkyl carboxamides, and the like.

Acceptable salts may be obtained using standard procedures well known in the art, for example by treating a sufficiently basic compound such as an amine with a suitable acid affording a physiologically acceptable anion. Alkali metal (for example, sodium, potassium or lithium) or alkaline earth metal (for example calcium) salts of organic (e.g., carboxylic) acids can also be made.

Compounds of according to embodiments described herein may have prodrug forms. Any compound that will be converted in vivo to provide the bioactive agent is a prodrug within the scope and spirit of the invention. Various forms of prodrugs are well known in the art (see, for example, Medicinal Chemistry: Principles and Practice, F. D. King, ed., The Royal Society of Chemistry, Cambridge, UK, 1994; Hydrolysis in Drug and Prodrug Metabolism. Chemistry, Biochemistry and Enzymology, B. Testa, J. M. Mayer, VCHA and Wiley-VCH, Zurich, Switzerland, 2003; The Practice of Medicinal Chemistry, C. G. Wermuth, 2^{nd} ed., Academic Press, San Diego, CA, 1999). Some prodrugs of the present invention include a compound according to any embodiment described herein in which the 2-hydroxy of the benzamide is converted to a group such as, but not limited to,

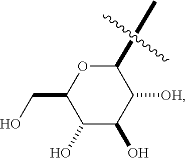

### and —CH₂OAc.

In some embodiments, a prodrug of a compound according to any embodiment described herein may take the form of a carbamate. For instance, the 2-hydroxy group of a benzamide according to any embodiment described may converted to a carbamate group, —OC(O)NR⁹R¹⁰ at the same position. Each of R⁹ and R¹⁰ is independently selected from the group consisting of hydrogen, and optionally substituted C₁-C₆-alkyl; alternatively R⁹ and R¹⁰ taken together with the nitrogen to which they are attached form an optionally substituted C₃-C₆-heterocyclyl.

The invention also embraces isolated compounds. An isolated compound refers to a compound which represents at least 10%, preferably at least 20%, more preferably at least 50% and most preferably at least 80% of the compound present in the mixture.

In some embodiments of the invention, one or more hydrogen atoms is replaced by a deuterium. It is well established that deuteration of physiologically active compounds offer the advantage of retaining the pharmacological profile of their hydrogen counterparts while positively impacting their metabolic outcome. Selective replacement of one or more hydrogen with deuterium, in a compound of the present invention, could improve the safety, tolerability and efficacy of the compound when compared to its all hydrogen counterpart.

Methods for incorporation of deuterium into compounds is well established. Using metabolic studies establish in the art, the compound of the present invention can be tested to identify sites for selective placement of a deuterium isotope, wherein the isotope will not be metabolized. Moreover these studies identify sites of metabolism as the location where a deuterium atom would be placed.

Some embodiments describe a pharmaceutical composition comprising: a compound according to an embodiment described herein, a pharmaceutically acceptable salt thereof, a solvate thereof, or a prodrug thereof; and a pharmaceutically acceptable carrier or diluent.

Compounds, or pharmaceutically acceptable salts thereof, can be formulated for oral, intravenous, intramuscular, subcutaneous or parenteral administration for the therapeutic or prophylactic treatment of diseases, disorders or infections described herein. For oral or parenteral administration, compounds of this invention can be mixed with conventional pharmaceutical carriers and excipients and used in the form of tablets, capsules, elixirs, suspensions, syrups, wafers and the like. The pharmaceutical compositions comprising a compound of this invention will contain from about 0.1 to about 99% by weight of the active compound, and more generally from about 10 to about 30%.

The pharmaceutical preparations disclosed herein are prepared in accordance with standard procedures and are administered at dosages that are selected to reduce, prevent or eliminate the infection (See, e. g., Remington's Pharmaceutical Sciences, Mack Publishing Company, Easton, Pa. and Goodman and Gilman's. The Pharmaceutical Basis of Therapeutics, Pergamon Press, New York, N.Y., the contents of which are incorporated herein by reference, for a general description of the methods for administering various agents for human therapy). The pharmaceutical compositions of the invention can be delivered using controlled (e.g., capsules) or sustained release delivery systems (e.g., bioerodable matrices).

The pharmaceutically acceptable pharmaceutical compositions of the present invention comprise one or more compounds of the invention in association with one or more non-toxic, pharmaceutically acceptable carriers and/or diluents and/or adjuvants and/or excipients, collectively referred to herein as “carrier” materials, and if desired other active ingredients. The pharmaceutical compositions may contain common carriers and excipients, such as corn starch or gelatin, lactose, sucrose, microcrystalline cellulose, kaolin, mannitol, dicalcium phosphate, sodium chloride and alginic acid. The pharmaceutical compositions may contain croscarmellose sodium, microcrystalline cellulose, corn starch, sodium starch glycolate and alginic acid.

Tablet binders that can be included are acacia, methylcellulose, sodium carboxymethylcellulose, polyvinylpyrrolidone (Povidone), hydroxypropyl methylcellulose, sucrose, starch and ethylcellulose.

Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. It may also be desirable to add a coloring agent to make the dosage form more aesthetic in appearance or to help identify the product.

For oral use, solid formulations such as tablets and capsules are particularly useful. Sustained release or enterically coated preparations may also be devised. For pediatric and geriatric applications, suspensions, syrups and chewable tablets are especially suitable. For oral administration, the pharmaceutical compositions are in the form of, for example, a tablet, capsule, suspension or liquid. The pharmaceutical composition is preferably made in the form of a dosage unit containing a therapeutically effective amount of the active ingredient. Examples of such dosage units are tablets and capsules. For therapeutic purposes, the tablets and capsules which can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example, potato starch, flavoring or coloring agents, or acceptable wetting agents. Oral liquid preparations generally are in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl parahydroxybenzoate, propylene glycol, sorbitol, or sorbic acid.

For intravenous (IV) use, a compound according to the invention can be dissolved or suspended in any of the commonly used intravenous fluids and administered by infusion. Intravenous fluids include, without limitation, physiological saline or Ringer's solution. Intravenous administration may be accomplished by using, without limitation, syringe, minipump or intravenous line.

Formulations for parenteral administration can be in the form of aqueous or non-aqueous isotonic sterile injection solutions or suspensions. These solutions or suspensions can be prepared from sterile powders or granules having one or more of the carriers mentioned for use in the formulations for oral administration. The compounds can be dissolved in polyethylene glycol, propylene glycol, ethanol, corn oil, benzyl alcohol, sodium chloride, and/or various buffers.

For intramuscular preparations, a sterile formulation of a compound or a suitable soluble salt form of the compound, for example the hydrochloride salt, can be dissolved and administered in a pharmaceutical diluent such as Water-for-Injection (WFI), physiological saline or 5% glucose. A suitable insoluble form of the compound may be prepared and administered as a suspension in an aqueous base or a pharmaceutically acceptable oil base, e.g., an ester of a long chain fatty acid such as ethyl oleate.

A dose of an intravenous, intramuscular or parental formulation of a compound may be administered as a bolus or by slow infusion. A bolus is a dose that is administered in less than 30 minutes. In a preferred embodiment, a bolus is administered in less than 15 or less than 10 minutes. In a more preferred embodiment, a bolus is administered in less than 5 minutes. In an even more preferred embodiment, a bolus is administered in one minute or less. An infusion is a dose that is administered at a rate of 30 minutes or greater. In a preferred embodiment, the infusion is one hour or greater. In another embodiment, the infusion is substantially constant.

For topical use the compounds of the present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

For application to the eyes or ears, the compounds of the present invention can be presented in liquid or semi-liquid form formulated in hydrophobic or hydrophilic bases as ointments, creams, lotions, paints or powders.

For rectal administration the compounds of the present invention can be administered in the form of suppositories admixed with conventional carriers such as cocoa butter, wax or other glyceride.

Alternatively, the compounds of the present invention can be in powder form for reconstitution in the appropriate pharmaceutically acceptable carrier at the time of delivery. In another embodiment, the unit dosage form of the compound can be a solution of the compound or preferably a salt thereof in a suitable diluent in sterile, hermetically sealed ampoules or sterile syringes. The concentration of the compound in the unit dosage may vary, e.g. from about 1 percent to about 50 percent, depending on the compound used and its solubility and the dose desired by the physician.

In some embodiments the pharmaceutical compositions described herein are at a therapeutically effective dosage level. In some embodiments, the pharmaceutical composition is at a therapeutically effective levels of between 0.001 to 100 mg/kg. of body weight daily for administration to a patient, e.g., humans and elderly humans. The therapeutically effective amount will generally be about 0.5 mg to 10 g per patient per day which may be administered in single or multiple doses. In some embodiments the therapeutically effective amount is between a lower limit of 0.5 mg, 10 mg, 1 mg, 500.0 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, and 10000 mg; and an upper limit of 10000 mg, 9500 mg, 9000 mg, 8500 mg, 8000 mg, 7500 mg, 7000 mg, 6500 mg, 6000 mg, 5500 mg, 5000 mg, 4500 mg, 4000 mg, 3500 mg, 3000 mg, 2500 mg, 2000 mg, 1500 mg, 1000 mg, 500.0 mg, 100 mg, 10 mg and 0.5 mg. In some embodiments, the therapeutically effective amount will be about 0.5 mg to 2500 mg per patient per day; in some embodiments about 0.5 mg to 200 mg per patient per day; in some embodiments about 0.5 mg to 500 mg per patient per day; in some embodiments about 0.5 mg to 1000 mg per patient per day; and in yet some other embodiments about 5 mg to 50 mg per patient per day. Pharmaceutical compositions of the present invention may be provided in a solid dosage formulation such as comprising about 0.5 mg to 500 mg active ingredient, or comprising about 1 mg to 250 mg active ingredient. The pharmaceutical composition may be provided in a solid dosage formulation comprising for example about 1 mg, 5 mg, 10 mg, 25 mg, 50 mg, 100 mg, 200 mg, 250 mg, 500 mg or 1000 mg of active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, 1000 and 2000 milligrams of the active ingredient for the symptomatic adjustment of the dosage to the patient to be treated. The compounds may be administered on a regimen of 1 to 4 times per day, such as once, twice, three times or four times per day.

### Unexpected Properties

Mitochondrial Uncouplers with Improved PK Profile

Some embodiments describe mitochondrial uncouplers depicted herein that have improved pharmacokinetic (PK) profiles, such as markedly increased systemic exposure (i.e. through increased solubility and absorption) consistent with drastically improved potency, and markedly reduced half-lives consistent with a once-daily oral administration regimen and elimination of the toxicity risk factor due to accumulation over prolonged use for chronic conditions or disorders.

A common feature of prior benzamide compounds with mitochondrial uncoupling activity is low systemic exposure. As shown in Table 1, niclosamide ethanolamine has an oral exposure of merely about 1800 hr*ng/ml in a 24 hr period after 50 mg/kg dosage. Compound 31 in International Patent Publication Number WO 2016/081599 and Compound 27 in U.S. Pat. No. 10,227,315 have systemic exposure of only about 2400 hr*ng/ml after 20 mg/kg oral administration and about 3000 hr*ng/ml after 10 mg/kg oral administration in 24 hr period of time respectively (Table 1). In order to overcome the poor pharmacokinetic properties of low systemic exposure and short half-life of the previously disclosed compounds, studies in animal models required the compound to be mixed with food, and high doses of the compounds were required to achieve efficacy (e.g. 1500 ppm niclosamide ethanolamine (NEN) in diet, equivalent to 150 mg/kg/day (Tao, et al. 2014, and WO 2012/068274); or 600-750 ppm (Compound 28 in U.S. Pat. No. 10,227,315) in diet, equivalent of 60-75 mg/kg/day). Neither the high doses required nor the need to mix the compound in food, is compatible with therapeutic development in humans.

As exemplified in Table 1, the new compounds (e.g., Compounds 2, 9, 78) unexpectedly exhibit significantly increased systemic exposure without compromising uncoupling activity, with 20- to 160-fold increase over the prior uncouplers with the best systemic exposure.

TABLE 1

| | | | Fold increase |
|---|---|---|---|
| | | | over |
| | | | Compound |
| | | Oral Exposure | 27 in U.S. |
| | | (AUC 24 hr) | Pat. No. |
| Compound | Where disclosed | (hr*ng/ml) | 10,227,315 |
| 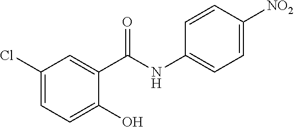 [SMILES: O=C(Nc1ccc([N+](=O)[O-])cc1)c1cc(Cl)ccc1O] | WO 2012/068274 | 1,725 ± 973 (50 mg/kg dose) | n.a. |
| Niclosamide | | | |
| 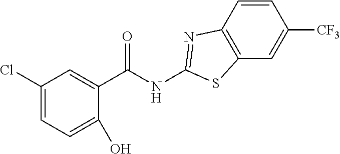 [SMILES: O=C(Nc1nc2ccc(C(F)(F)F)cc2s1)c1cc(Cl)ccc1O] | WO 2016/081599 | 2,357 ± 261 (20 mg/kg dose) | n.a. |
| Compound 31 | | | |
| 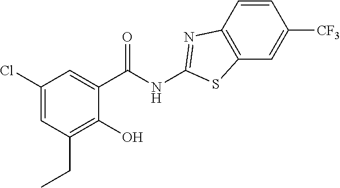 [SMILES: CCc1cc(Cl)cc(C(=O)Nc2nc3ccc(C(F)(F)F)cc3s2)c1O] | U.S. Pat. No. 10,227,315 | 2,982 ± 1,746 (10 mg/kg dose) | 1.0 |
| Compound 27 | | | |
| 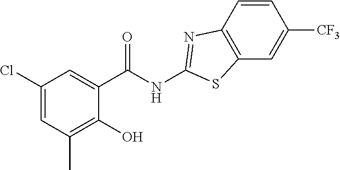 [SMILES: Cc1cc(Cl)cc(C(=O)Nc2nc3ccc(C(F)(F)F)cc3s2)c1O] | U.S. Pat. No. 10,227,315 | 4,413 ± 601 (10 mg/kg dose) | 1.5 |
| ethanolamine salt | | | |
| Compound 28 | | | |
| 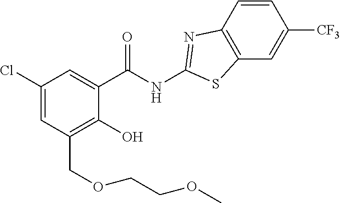 [SMILES: COCCOCc1cc(Cl)cc(C(=O)Nc2nc3ccc(C(F)(F)F)cc3s2)c1O] | Present Application | 484,954 ± 31,834 (10 mg/kg dose) | 163 |
| Compound 2 | | | |
| 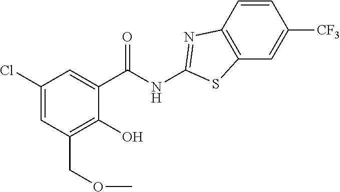 [SMILES: COCc1cc(Cl)cc(C(=O)Nc2nc3ccc(C(F)(F)F)cc3s2)c1O] | Present Application | 180,003 ± 67,785 (10 mg/kg dose) | 60.3 |
| Compound 9 | | | |
| 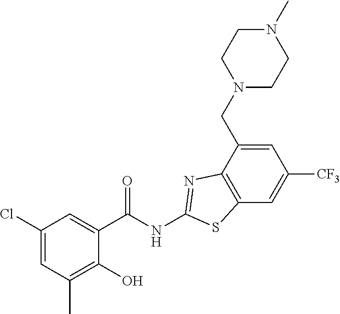 [SMILES: Cc1cc(Cl)cc(C(=O)Nc2nc3c(CN4CCN(C)CC4)cc(C(F)(F)F)cc3s2)c1O] | Present Application | 58,392 ± 24,757 (10 mg/kg dose) | 19.6 |
| Compound 78 | | | |

Table 2 provides additional compounds with drastically increased systemic exposure (See Example B3).

TABLE 2

| Compound | Oral Exposure (AUC, hr*ng/ml, |
|---|---|
| Number | 24 hrs) after 10 mg/kg gavage |
| 2 | ++++++ |
| 3 | +++ |
| 4 | ++++ |
| 9 | ++++++ |
| 11 | +++++ |
| 13 | ++++ |
| 14 | ++++++ |
| 15 | ++++++ |
| 16 | +++ |
| 17 | +++ |
| 23 | +++ |
| 25 | +++ |
| 26 | +++ |
| 27 | ++++ |
| 32 | ++++ |
| 64 | +++ |
| 65 | +++++ |
| 66 | ++++ |
| 68 | ++++ |
| 69 | +++ |
| 78 | ++++ |
| 97 | +++ |

+++: 10,000-30,000;
++++: 30,001-60,000;
+++++: 60,001-120,000;
++++++: >120,000

As noted in Table 3, Mitochondrial uncoupling activity of Compound 49 in U.S. Pat. No. 10,227,315 is low compared to similar compounds of the present invention.

TABLE 3

| | | Activity |
|---|---|---|
| Structure | Where disclosed | (C_{min-OCR,} μM) |
| 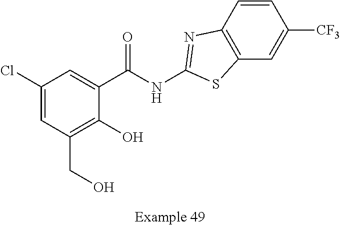 [SMILES: O=C(Nc1nc2ccc(C(F)(F)F)cc2s1)c1cc(Cl)cc(CO)c1O] | U.S. Pat. No. 10,227,315 | 25 |
| 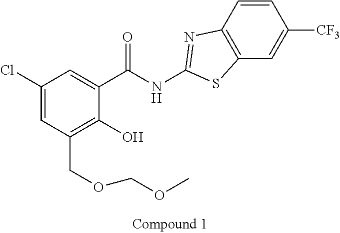 [SMILES: COCOCc1cc(Cl)cc(C(=O)Nc2nc3ccc(C(F)(F)F)cc3s2)c1O] | Present Application | 2.5 |
| 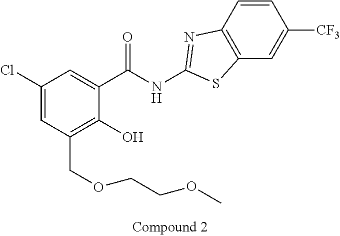 [SMILES: COCCOCc1cc(Cl)cc(C(=O)Nc2nc3ccc(C(F)(F)F)cc3s2)c1O] | Present Application | 2.5 |

### Improved Metabolic Stability and Half-Life Properties

Some embodiments describe compounds depicted herein with improved metabolic stability and half-life.

One key reason previously known mitochondrial uncouplers such as DNP exhibit a narrow therapeutic index is due to their long-half life. For example, DNP has a half-life measured in days to weeks, leading to accumulation of toxic levels in the body during prolonged use to treat chronic conditions or disorders. For development of orally administered drugs, excretion properties are also important. Not only does a drug need to have sufficient exposure, but it also needs to be excreted at a rate that is not too short nor to long. On the one hand, it is important that an oral drug should have sufficiently long half-life in patients so the drug can be administered at certain intervals (e.g. once daily instead of once every hour); on the other hand, the half-life of a compound cannot be too long as this could lead to toxic accumulation of the drug inside the patient. For once-daily orally administered drugs, a half-life around 8-12 hrs is considered a favorable property, while half-lives exceeding 48 hrs are incompatible for once-daily oral administration and could lead to toxic accumulation of the drug. For example, Compound 2 of U.S. Pat. No. 10,227,315 exhibits an extremely long half-life (66.9 hrs, Table 4, Example B8). The long half-life correlated with extreme metabolic stability (rat microsome metabolic stability, half-life 2,131.8 minutes). In other words, the extremely long half-life of Compound 2 of U.S. Pat. No. 10,227,315 is mainly caused by the liver metabolic enzymes inability to efficiently metabolize these compounds.

As shown in Table 4, Compound 17 of the present invention unexpectedly has drastically decreased metabolic stability (from 2,132 minutes to 145 minutes, Example B7), and concomitant decreased oral half-life (from 66.9 hrs to 8.6 hrs, Example B8). The structural modification does not decrease the mitochondrial uncoupling activity thus are expected to have better toxicology properties when used to treat chronic conditions or disorders, which require prolonged use.

TABLE 4

| Structure | 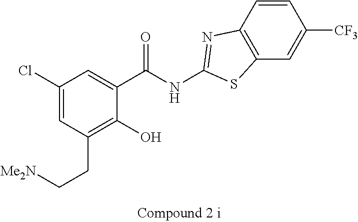 [SMILES: CN(C)CCc1cc(Cl)cc(C(=O)Nc2nc3ccc(C(F)(F)F)cc3s2)c1O] | 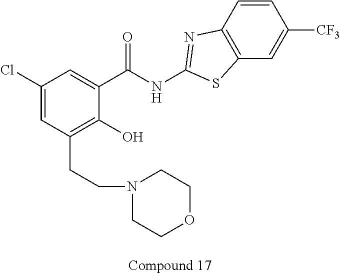 [SMILES: O=C(Nc1nc2ccc(C(F)(F)F)cc2s1)c1cc(Cl)cc(CCN2CCOCC2)c1O] |
|---|---|---|
| Where Disclosed | U.S. Pat. No. 10,227,315 | Present Application |
| Metabolic stability half- | 2132 | 145 |
| life (T_{1/2}, min, rat liver | | |
| microsome) | | |
| Rat Oral PK (in vivo) | 66.9 | 8.6 |
| half-life (T_{1/2}, hour) | | |

Half-lives in rat liver microsomal stability assay were determined (Example B7) for a number of compounds described herein and compared with Compound 2, Compound 53, and 54 in U.S. Pat. No. 10,227,315 (Table 5). The compounds of the present invention exhibited drastically improved metabolic stability (favorable half-life less likely to cause toxicity when used to treat chronic conditions or disorders, which require prolonged use) without decreased uncoupling activity (see Table 5 and Table 8).

TABLE 5

| | | Metabolic stability |
|---|---|---|
| | | half-life (T_{1/2}, min, rat |
| Compound | Where disclosed | liver microsome) |
| 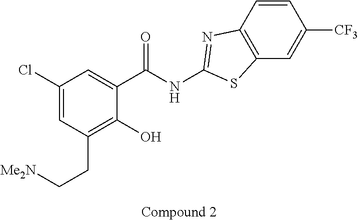 [SMILES: CN(C)CCc1cc(Cl)cc(C(=O)Nc2nc3ccc(C(F)(F)F)cc3s2)c1O] | U.S. Pat. No. 10,227,315 | 2,132 |
| 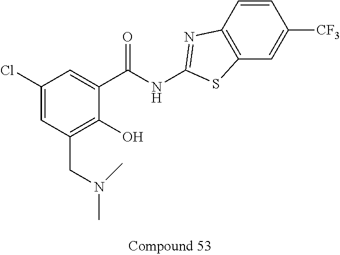 [SMILES: CN(C)Cc1cc(Cl)cc(C(=O)Nc2nc3ccc(C(F)(F)F)cc3s2)c1O] | U.S. Pat. No. 10,227,315 | 882 |
| 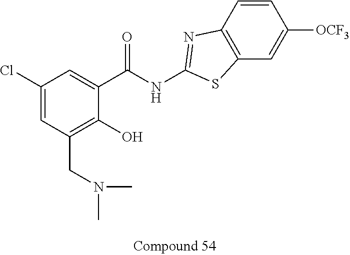 [SMILES: CN(C)Cc1cc(Cl)cc(C(=O)Nc2nc3ccc(OC(F)(F)F)cc3s2)c1O] | U.S. Pat. No. 10,227,315 | ∞ |
| 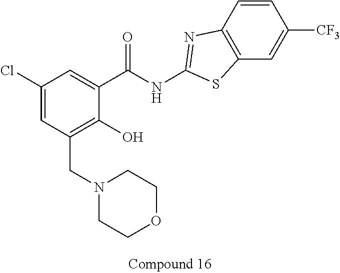 [SMILES: O=C(Nc1nc2ccc(C(F)(F)F)cc2s1)c1cc(Cl)cc(CN2CCOCC2)c1O] | Present Application | 90 |
| 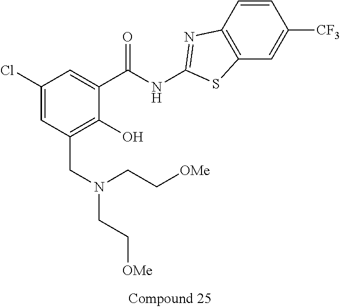 [SMILES: COCCN(CCOC)Cc1cc(Cl)cc(C(=O)Nc2nc3ccc(C(F)(F)F)cc3s2)c1O] | Present Application | 28 |
| 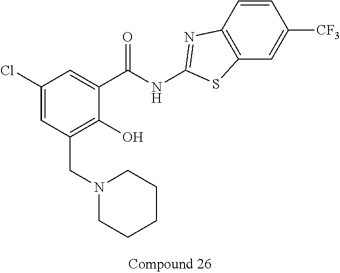 [SMILES: O=C(Nc1nc2ccc(C(F)(F)F)cc2s1)c1cc(Cl)cc(CN2CCCCC2)c1O] | Present Application | 92 |

### MMP-Retaining Compounds

Some embodiments describe mitochondrial uncouplers depicted herein that effectively increases OCR without reducing or significantly reducing MMP. These compounds are referred to as MMP-retaining uncouplers in this disclosure.

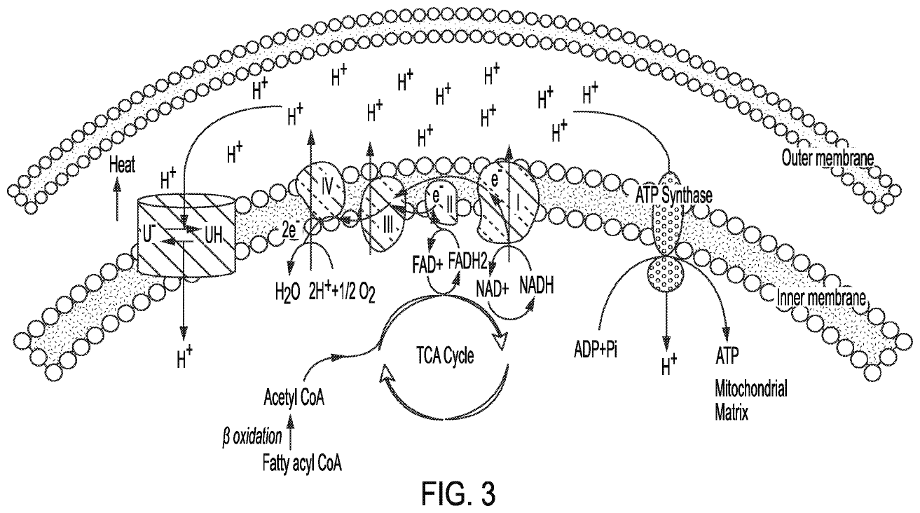

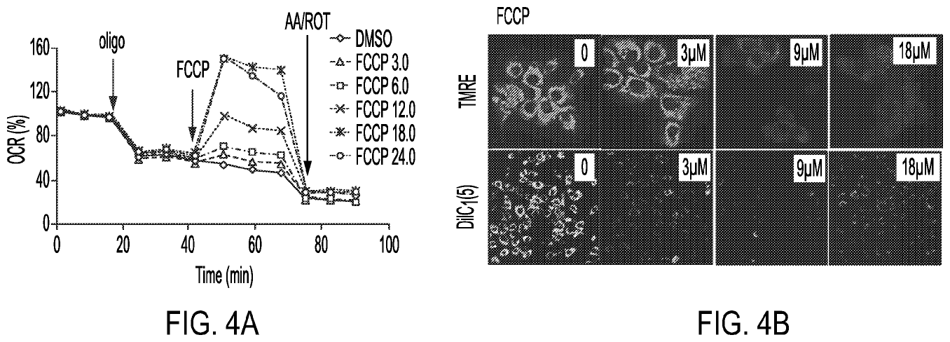

Mechanistically, conventional mitochondrial uncouplers transport protons across the mitochondrial inner membrane into the mitochondrial matrix. As mitochondrial membrane potential is supported by proton gradient across the membrane, conventional uncouplers inevitably require rapid dissipation of mitochondrial membrane potential to sustain mitochondrial uncoupling action (FIG. 3). Prior to the present disclosure, all mitochondrial uncouplers (herein referred to as conventional uncouplers) tested, had properties that increase OCR and decreased MMP, and the concentrations leading to OCR increase and MMP dissipation correlate (FIG. 4). FIG. 4 shows a conventional uncoupler, FCCP, has a ratio of C_{10% TMRE}/C_{min-OCR} less than 3, where C_{10% TMRE} is the concentration leading to 10% MMP retention (or 90% MMP loss, measured by TMRE staining) and C_{min-OCR} is the minimal concentration leading to OCR increase. As such, the activity for decreasing MMP is considered a second hallmark of mitochondrial uncoupling. As MMP is critical for cell survival and normal functions of cells in many tissues and organs, this seemingly inherent property of conventional mitochondrial uncouplers on dissipating MMP represents a major safety hurdle for therapeutic development.

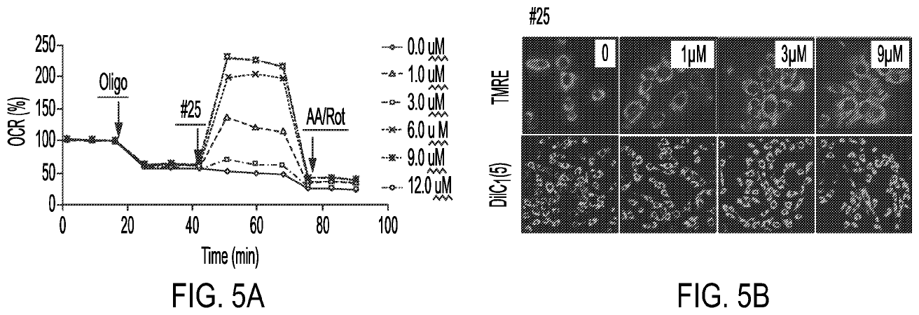

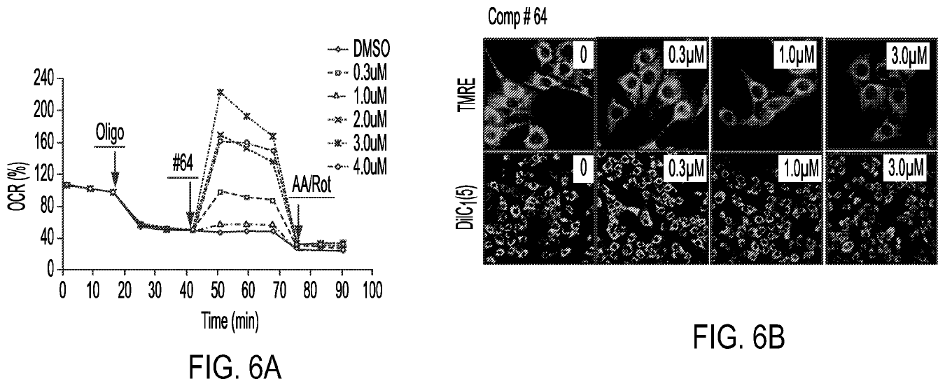

In some embodiments, the mitochondrial uncouplers described herein represent a fundamentally different category of mitochondrial uncouplers which effectively induce mitochondrial uncoupling (increase mitochondrial oxygen consumption in the presence of oligomycin) without significantly decreasing mitochondrial membrane potential over a wide concentration range (FIGS. 5 and 6, Example B2). FIG. 5 shows that Compound 25 does not appear to reduce MMP over a wide concentration range where OCR increases and reaches maximal levels. The ratio of C_{10% TMRE}/C_{min-OCR} for Compound 25 is over 25. FIG. 6 shows that Compound 64 effectively induces mitochondrial uncoupling without significantly decreasing MMP over a wide concentration range. The ratio of C_{10% TMRE}/C_{min-OCR} for Compound 64 is between 10 to 25.

After determining the mitochondrial uncoupling activity of a mitochondrial uncoupler described herein (Example B1), a mitochondrial membrane potential assay was performed on the mitochondrial uncoupler, using the standard TMRE (tetramethylrhodamine ethyl ester) staining method with cultured mammalian cells (Example B2). Results are categorized into three groups, the Compound 25-like compounds are denoted as MMP-Retaining Uncoupling Compounds (C_{10% TMRE}/C_{min-OCR}>25); The Compound 64-like compounds are denoted as MMP-Retaining Uncoupling Compounds (C_{10% TMRE}/C_{min-OCR} between 10 to 25), and Conventional Uncouplers (C_{10% TMRE}/C_{min-OCR} less or equal to 3). The results are summarized in Table 6.

TABLE 6

| MMP-Retaining | MMP-Retaining | |
|---|---|---|
| Uncoupling | Uncoupling | Conventional |
| Compounds | Compounds | Uncouplers |
| C_{10%TMRE}/ | C_{10%TMRE}/ | C_{10%TMRE}/ |
| C_{min-OCR} >25 | C_{min-OCR} 10-25 | C_{min-OCR}~3 |
| 3 | 16 | 1 |
| 18 | 17 | 2 |
| 22 | 33 | 4 |
| 23 | 64 | 5 |
| 24 | 65 | 6 |
| 25 | 70 | 8 |
| 26 | 72 | 9 |
| 27 | 91 | 11 |
| 28 | 92 | 13 |
| 32 | | 14 |
| 53 | | 15 |
| 58 | | 36 |
| 59 | | 37 |
| 60 | | 43 |
| 62 | | 45 |
| 66 | | 46 |
| 69 | | 47 |
| 74 | | 48 |
| 75 | | 49 |
| 76 | | 51 |
| 78 | | 55 |
| 79 | | 56 |
| 93 | | 57 |
| | | 61 |
| | | 63 |
| | | 67 |
| | | 68 |
| | | 71 |
| | | 82 |
| | | 83 |
| | | 84 |
| | | 85 |
| | | 88 |
| | | 89 |
| | | 90 |
| | | 94 |
| | | 95 |
| | | 96 |
| | | 97 |
| | | 98 |

C_{10%TMRE} is the concentration leading to 10% MMP retention (or 90% MMP loss, measured by TMRE staining) and C_{min-OCR} is the minimal concentration leading to OCR increase

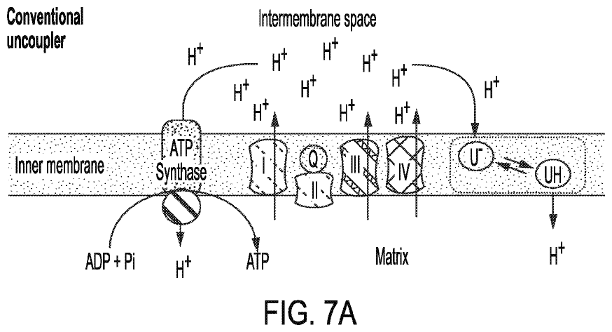

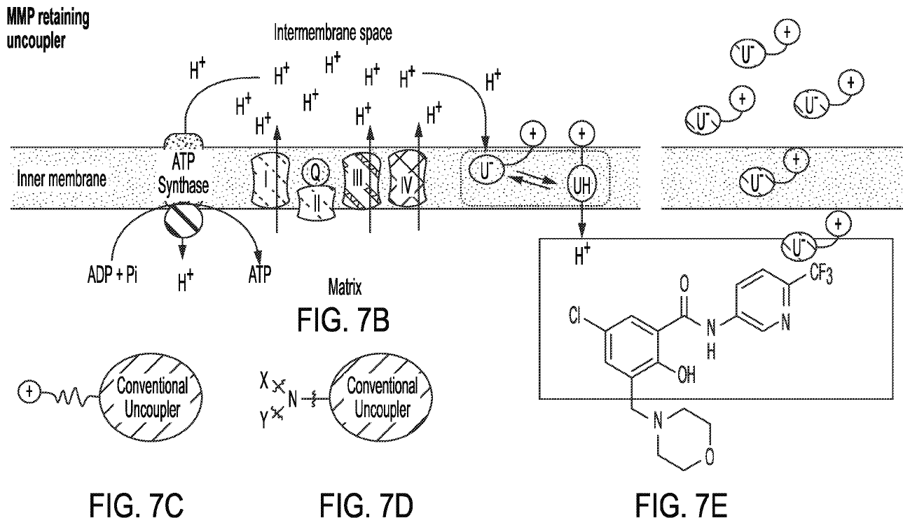

Mechanistically, chemical uncouplers are lipophilic weak acids or weak bases that localize in mitochondrial inner membrane and transport protons across the membrane into mitochondrial matrix through a protonation and deprotonation cycle (FIG. 3 and FIG. 7A). As mitochondrial membrane potential (MMP) is supported by proton gradient across the membrane, it is highly unexpected that a compound could uncouple mitochondrial oxidation (transport protons) without significantly decreasing MMP. We analyzed the benzothiazole derivatives of benzamide uncouplers that exhibit MMP-retaining activity. They all contain a tertiary amine or secondary amine group, which have a pKa over 10.0. The amine groups become positively charged in cellular environment where the pH is generally below 8.0.

Without wishing to be bound by theory, we propose the mechanism of action for the MMP-retaining uncouplers, is as illustrated in FIG. 7B. In essence, a MMP-retaining uncoupler molecule consists of two functional parts (FIG. 7C). The first part functions as a conventional uncoupler, which transports proton from the mitochondrial intermembrane space to the mitochondrial matrix (FIG. 7B). The second part is a positively charged functional group that is poorly impermeable to the mitochondrial inner membrane. This feature allows an asymmetrical distribution and orientation of the compounds across mitochondrial inner membrane, with higher concentration of the charged molecules in the intramembrane space than in the mitochondrial matrix, as well as with the positively charged moiety primarily distributed at the outer surface of the membrane (facing the intermembrane space, FIG. 7B). As mitochondrial uncoupling occurs and consequently reduction of proton gradient across the membrane, the loss of membrane potential due to proton gradient reduction is compensated by the asymmetrical positive charge distribution across the membrane provided by MMP-retaining uncouplers. Hence, the overall mitochondrial membrane potential is minimally impacted over a wide concentration range of the uncouplers.

FIG. 7D illustrates the structural feature of the all the MMP-retaining uncouplers listed in Table 6 in which the positive charge is provided by a tertiary amine or a secondary amine. We notice that by modifying the amine containing moiety, one could fine-tune the MMP-retaining activity, reducing the ratio of C_{10% TMRE}/C_{min-OCR} from >25 to a ratio between 10 to 25. Some modification of the amine-containing moiety could even reduce the ratio close to 3, a ratio equal to that of a typical conventional mitochondrial uncoupler. FIG. 7E shows a specific example of the MMP-retaining uncoupler, with the conventional uncoupler component shown in the frame.

### Improved In Vivo Safety Profiles

Some embodiments describe MMP-retaining uncouplers exhibit markedly improved safety profiles compared with conventional uncouplers such as the bench mark conventional uncoupler DNP.

DNP is a conventional mitochondrial uncoupler once used in humans before for obesity treatment. However, it exhibited high levels of toxicity and had a narrow therapeutic window. It was subsequently withdrawn from market for use in humans. The toxicology profiles of the MMP-retaining uncouplers using Compound 25 and Compound 64 as examples, and compared them with those of DNP. Compounds 25 and Compound 64 have excellent oral absorption, systemic exposure, and efficacy in reducing blood glucose and liver steatosis in animal models (Tables 2, 5, and 9).

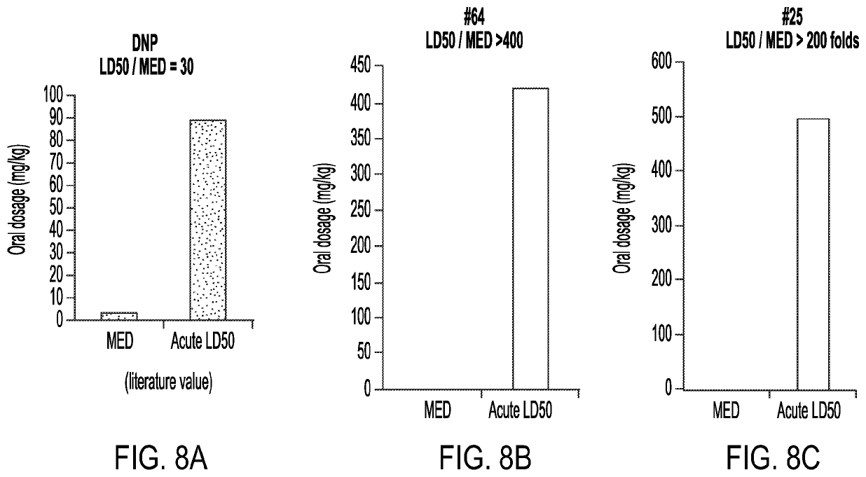

Some embodiments show MMP-retaining uncouplers exhibit markedly improved acute toxicity profiles. FIG. 8 (Example B9) shows Compound 64 and Compound 25 have LD50 (lethal dose, 50% animal death)/IED (minimal efficacious dosage) ratios over 400 and 200 respectively, while the LD50/MED ratio of DNP is 30.

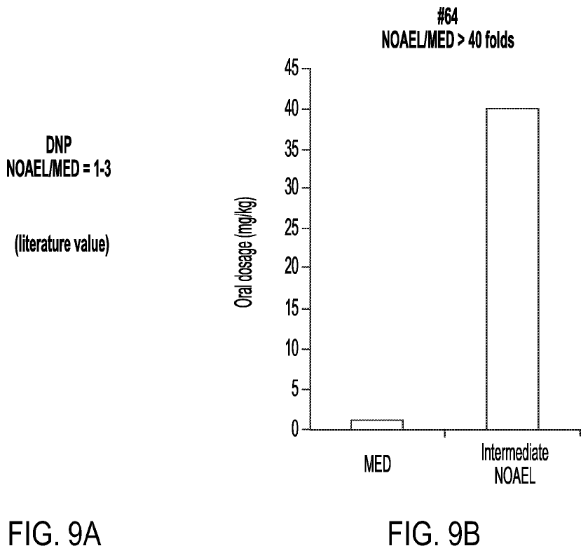

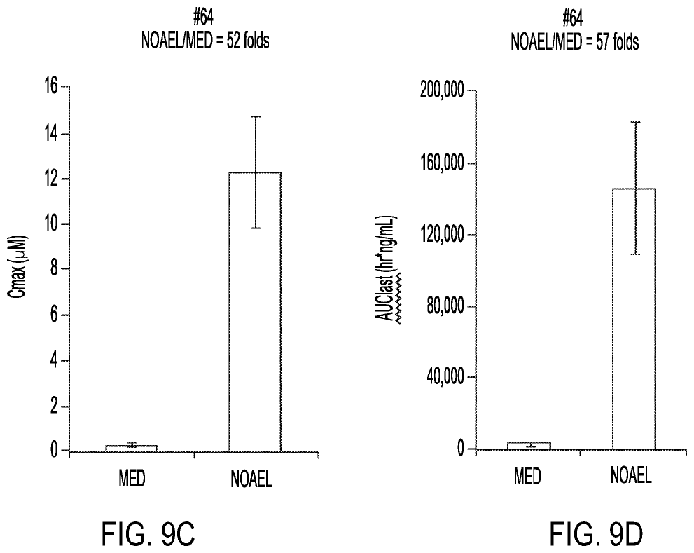

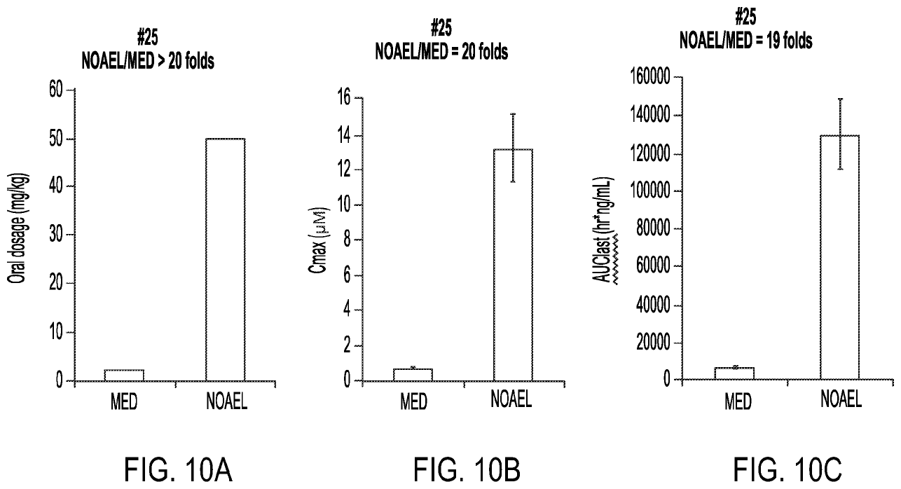

Some embodiments show MMP-retaining uncouplers exhibiting markedly improved short-term (10-day) toxicology profiles. FIG. 9-10 (Example B10) show that the ratio of NOAELs (no-observable-adverse-effect-level) over MED of Compounds 64 is over 40 (between 40-57 using different parameters), NOAEL/MED ratio of Compound 25 is over 19, while NOAEL/MED ratio of DNP is reportedly less than 3.

Some embodiments describe a method of preparing MMP-retaining uncouplers from conventional mitochondrial uncouplers by adding a positively charged side chain to the conventional uncouplers, as illustrated in FIG. 7C, for example, by adding a tertiary amine (or secondary amine) containing side chain (FIG. 7D). As shown in FIG. 7E, a tertiary amine-containing moiety was added to a conventional uncoupler (the structure in the Frame). This modification effectively converts the conventional uncoupler (with the ratio of C_{10% TMRE}/C_{min-OCR} of 3) to an MMP-retaining uncoupler (listed in Table 2, with the ratio of C_{10% TMRE}/C_{min-OCR} over 25).

Some embodiments describe a mitochondrial membrane-retaining uncoupler compound of the Formula:

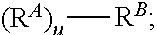

or a pharmaceutically acceptable salt, solvate or prodrug thereof;
wherein R^{A} and R^{B} are covalently linked;
each R^{A} is independently a moiety containing a secondary or tertiary amine;
u is an integer selected from the group consisting of 1 and 2; and
R^{B} is a conventional mitochondrial uncoupler prior to being covalently linked to R^{A};
provided the mitochondrial membrane-retaining uncoupler compound is not

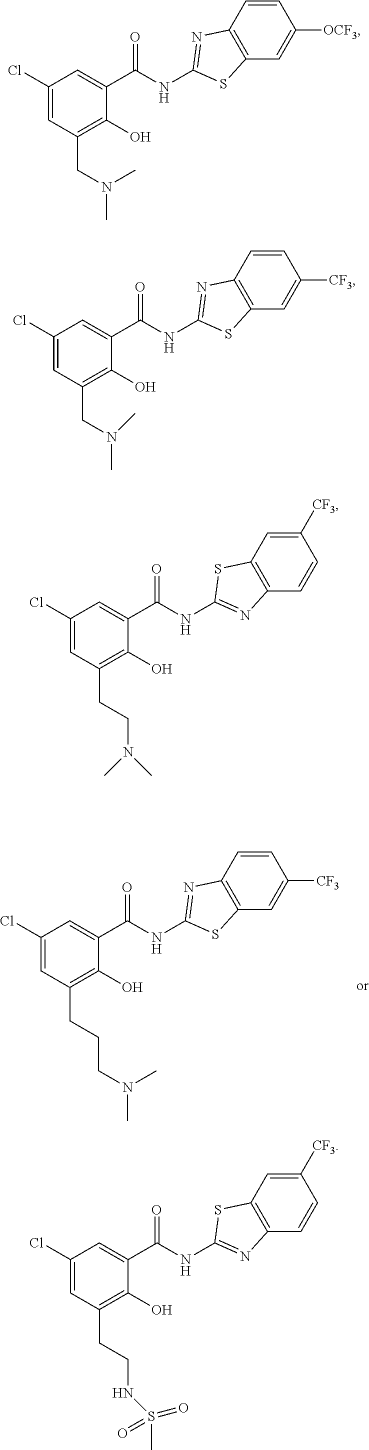 [SMILES: CN(C)Cc1cc(Cl)cc(C(=O)Nc2nc3ccc(OC(F)(F)F)cc3s2)c1O ; CN(C)Cc1cc(Cl)cc(C(=O)Nc2nc3ccc(C(F)(F)F)cc3s2)c1O ; CN(C)CCc1cc(Cl)cc(C(=O)Nc2nc3ccc(C(F)(F)F)cc3s2)c1O ; CN(C)CCCc1cc(Cl)cc(C(=O)Nc2nc3ccc(C(F)(F)F)cc3s2)c1O ; CS(=O)(=O)NCCc1cc(Cl)cc(C(=O)Nc2nc3ccc(C(F)(F)F)cc3s2)c1O]

Some embodiments describe a method of preparing a mitochondrial membrane-retaining mitochondrial uncoupler comprising
1. identifying a conventional mitochondrial uncoupler;
2. designing a compound that covalently links at least one moiety capable of becoming positively charge in a cellular environment, to a conventional mitochondrial uncoupler; and
3. preparing the compound of step 2, wherein the compound is a mitochondrial membrane retaining uncoupler compound.
   In some embodiments the moiety capable of becoming positively charge in a cellular environment is a secondary or tertiary amine moiety.

Some embodiments describe a method of preparing a mitochondrial membrane-retaining mitochondrial uncoupler comprising
1. identifying a conventional mitochondrial uncoupler;
2. designing a compound that covalently links at least one secondary or tertiary amino moiety to a conventional mitochondrial uncoupler; and
3. preparing the compound of step 2, wherein the compound is a mitochondrial membrane retaining uncoupler compound.

In some embodiments the secondary or tertiary amino moiety or R^{A} is selected from the group consisting of —(CH₂)NH(CH₂)₂OCH₃, —CH₂NHC(O)CH₃, —(CH₂)NHCO₂CH₃, —CH₂NHSO₂CH₃

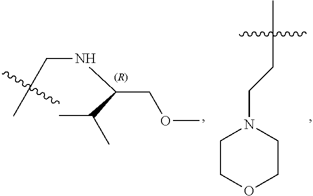

—(CH₂)_{r′}NR^{5000A}R^{5000B}, (CH₂)_{m′}NR^{5A}R^{5B},
—(CH₂)_{o′}NR⁵⁰⁰R^{500B}, —(CH₂)_{p′}NR^{500A}R^{500B}, —CH₂O(CH₂)₂NHCH₃, —CH₂O(CH₂)₂N(CH₃)₂, —CH₂O(CH₂)₂NHSO₂CH₃, —(CH₂)O(CH₂)₂NR^{2A}R^{2B}, —(CH₂)O(CH₂)₂NR^{2000A}R^{2000B}, —(CH₂)_{q′}NR^{7A}R^{7B}, and —(CH₂)_{t′}NR^{70000A}R^{7000B} wherein R^{5000A}, R^{5000B}, R^{5A}, R^{5B}, R^{50A}, R^{50B}, R^{500A}, R^{500B}, R^{2A}, R^{2B}, R^{2000A}, R^{2000B}, R^{7A}, R^{7B}, R^{7000A}, R^{7000B}, r′, m′, o′ p′, q′, and t′ are as previously described in any embodiment described herein.

In some embodiments the secondary or tertiary amino moiety or R^{A} is selected from the group consisting of —CH₂NHSO₂CH₃, —CH₂N(CH₃)₂, —(CH₂)₂N(CH₃)₂, —(CH₂)NH(CH₂)₂OCH₃, —CH₂NHC(O)CH₃, —CH₂NHC(O)CH₂CH₃,

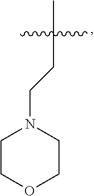 [SMILES: *CCN1CCOCC1]

—CH₂N(CH₃)(CH₂)₂OCH₃, —(CH₂)N(CH₂CH₂OCH₃)₂, —CH₂N(CH₃)(CH₂)₂O(CH₂)₂OCH₃,

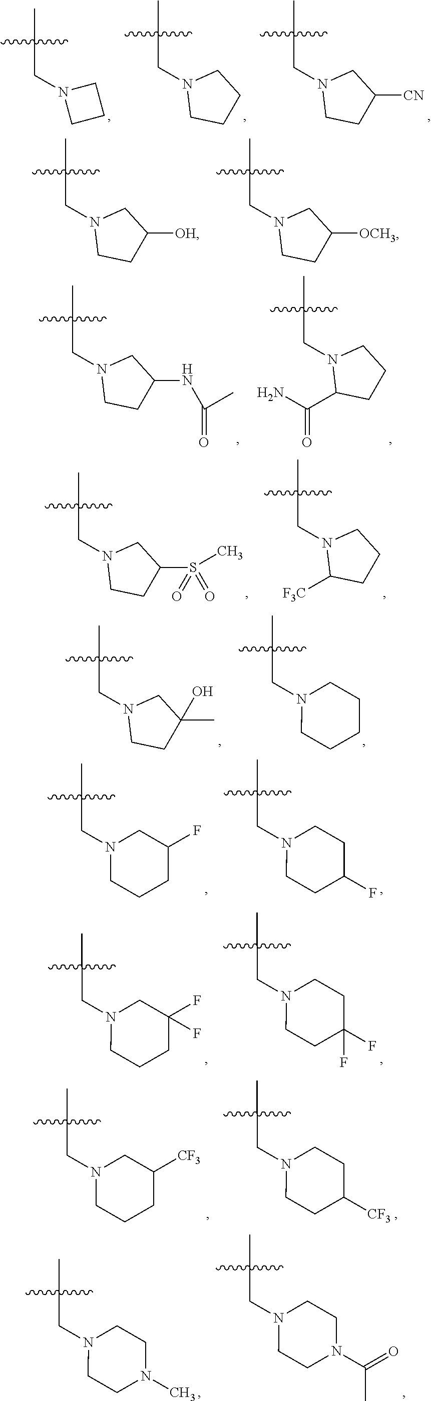

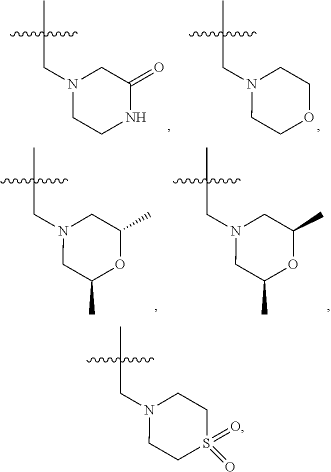

C(O)N(CH₂CH₂OCH₃)₂, —CH₂O(CH₂)₂N(CH₃)₂, —CH₂O(CH₂)₂NHSO₂CH₃,

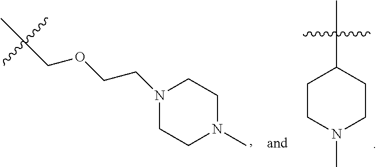

In some embodiments the secondary or tertiary amino moiety or R^{A} is selected from the group consisting of

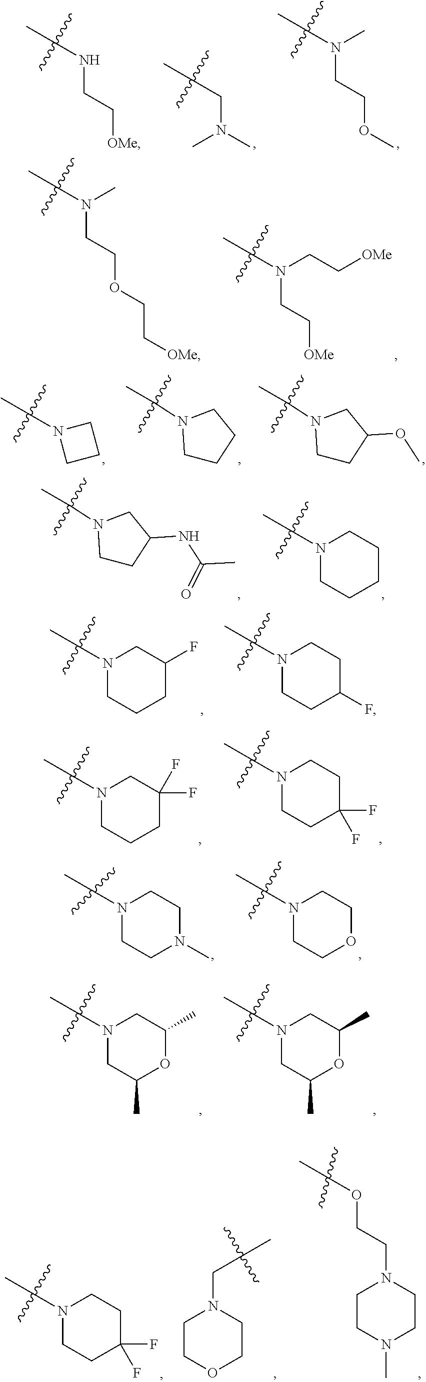

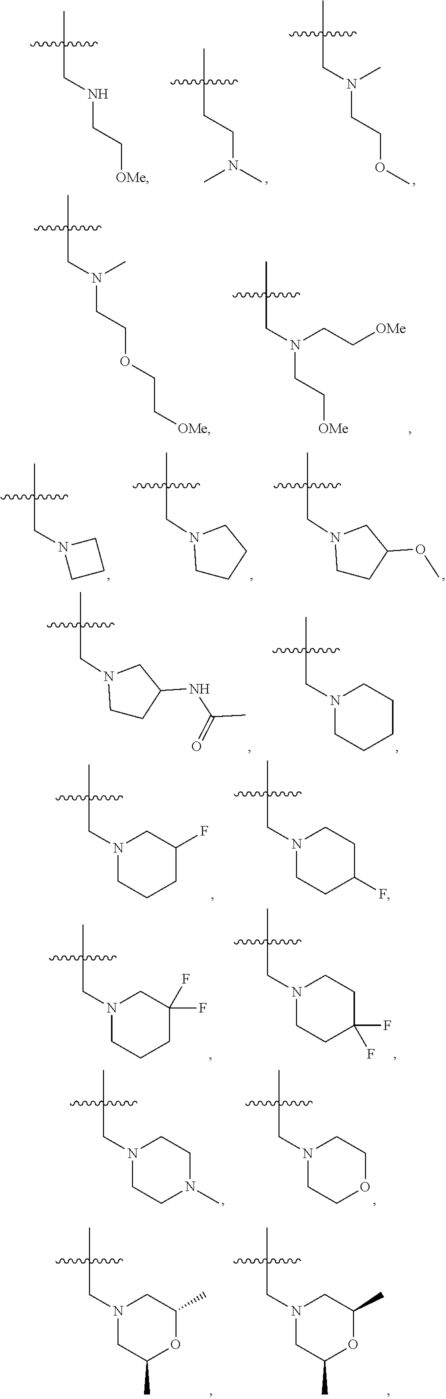

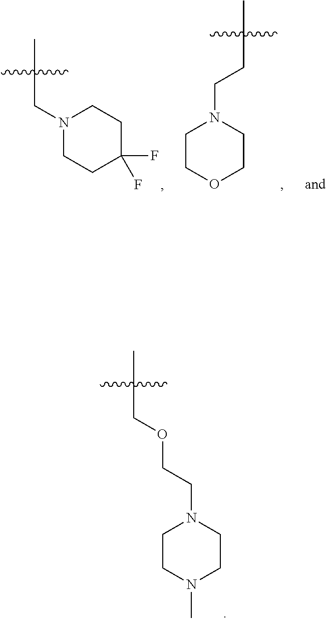

In some embodiments the conventional mitochondrial uncoupler or R^{B} is selected from the group consisting of a conventional mitochondrial uncoupler as described herein. In some embodiments the conventional mitochondrial uncoupler or R^{B} is selected from the group consisting of a conventional mitochondrial uncoupler as described in: U.S. Pat. No. 10,227,3158, U.S. patent application Ser. No. 15/527,808 and Childress, E. S., et al. (2018) J. Med Chem., 61(11), 4641-4655.

In some embodiments the conventional mitochondrial uncoupler or R^{B} is selected from the group consisting of

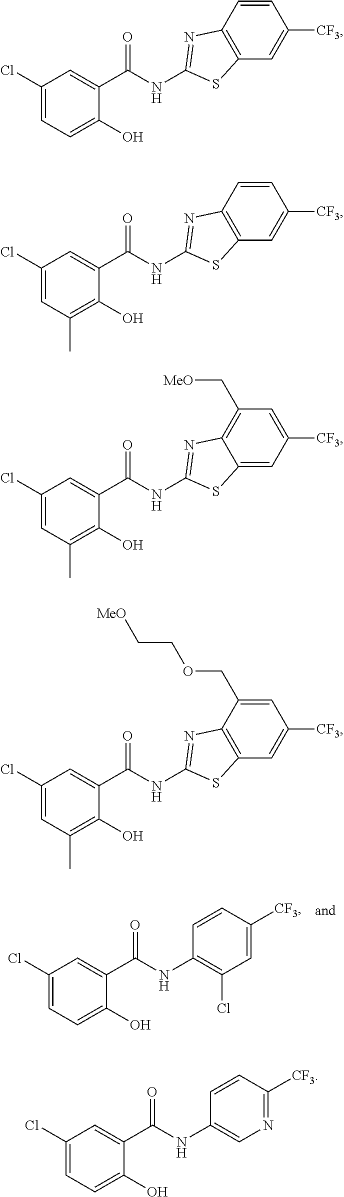 [SMILES: O=C(Nc1nc2ccc(C(F)(F)F)cc2s1)c1cc(Cl)ccc1O ; Cc1cc(Cl)cc(C(=O)Nc2nc3ccc(C(F)(F)F)cc3s2)c1O ; COCc1cc(C(F)(F)F)cc2sc(NC(=O)c3cc(Cl)cc(C)c3O)nc12 ; COCCOCc1cc(C(F)(F)F)cc2sc(NC(=O)c3cc(Cl)cc(C)c3O)nc12 ; O=C(Nc1ccc(C(F)(F)F)cc1Cl)c1cc(Cl)ccc1O ; O=C(Nc1ccc(C(F)(F)F)nc1)c1cc(Cl)ccc1O]

In some embodiments the secondary or tertiary amino moiety or R^{A}; the conventional mitochondrial uncoupler or R^{B}; and the mitochondrial membrane-retaining uncoupler compound (R^{A})ᵤ—R^{B} are as described in Table 7.

TABLE 7

| | Corresponding | |
|---|---|---|
| | moiety | |
| | containing | |
| | tertiary or | |
| Conventional Uncoupler or R^{B} | secondary amine | MMP-Retaining Uncoupling |
| as defined herein | group or R^{A} as | Compounds, (R^{A})ᵤ-R^{B} |
| (C_{10%TMRE}/C_{min-OCR} = ~3) | defined herein | (C_{10%TMRE}/C_{min-OCR} > 10) |
| 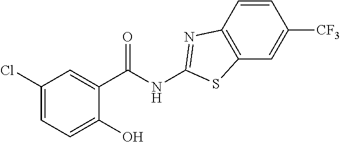 [SMILES: O=C(Nc1nc2ccc(C(F)(F)F)cc2s1)c1cc(Cl)ccc1O] | 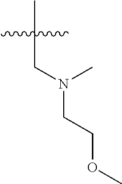 [SMILES: *CN(C)CCOC] | 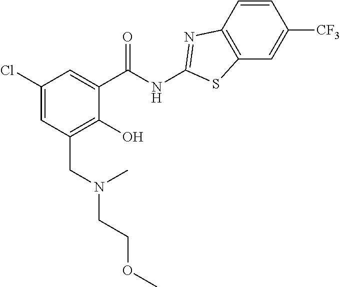 [SMILES: COCCN(C)Cc1cc(Cl)cc(C(=O)Nc2nc3ccc(C(F)(F)F)cc3s2)c1O] |
| | | 3 |
| 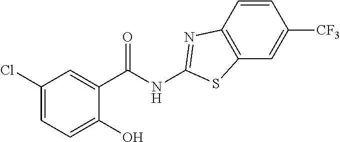 [SMILES: O=C(Nc1nc2ccc(C(F)(F)F)cc2s1)c1cc(Cl)ccc1O] | 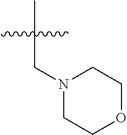 [SMILES: *CN1CCOCC1] | 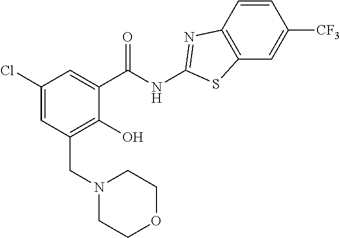 [SMILES: O=C(Nc1nc2ccc(C(F)(F)F)cc2s1)c1cc(Cl)cc(CN2CCOCC2)c1O] |
| | | 16 |
| 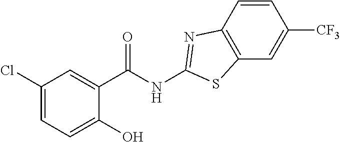 [SMILES: O=C(Nc1nc2ccc(C(F)(F)F)cc2s1)c1cc(Cl)ccc1O] | 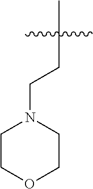 [SMILES: *CCN1CCOCC1] | 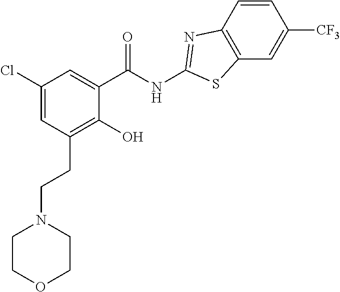 [SMILES: O=C(Nc1nc2ccc(C(F)(F)F)cc2s1)c1cc(Cl)cc(CCN2CCOCC2)c1O] |
| | | 17 |
| 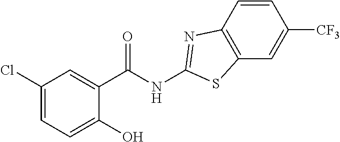 [SMILES: O=C(Nc1nc2ccc(C(F)(F)F)cc2s1)c1cc(Cl)ccc1O] | 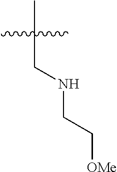 [SMILES: *CNCCOC] | 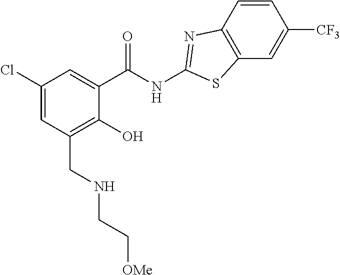 [SMILES: COCCNCc1cc(Cl)cc(C(=O)Nc2nc3ccc(C(F)(F)F)cc3s2)c1O] |
| | | 22 |
| 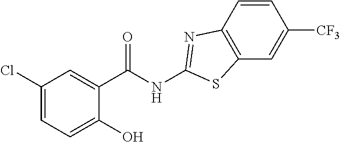 [SMILES: O=C(Nc1nc2ccc(C(F)(F)F)cc2s1)c1cc(Cl)ccc1O] | 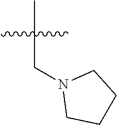 [SMILES: *CN1CCCC1] | 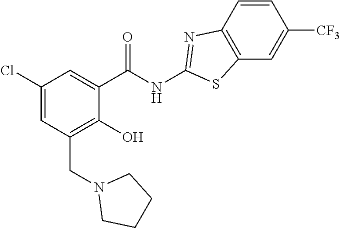 [SMILES: O=C(Nc1nc2ccc(C(F)(F)F)cc2s1)c1cc(Cl)cc(CN2CCCC2)c1O] |
| | | 23 |
| 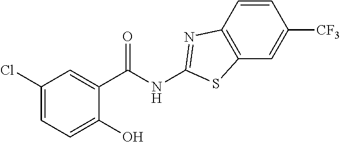 [SMILES: O=C(Nc1nc2ccc(C(F)(F)F)cc2s1)c1cc(Cl)ccc1O] | 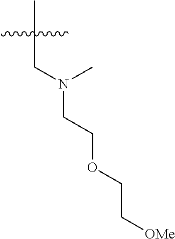 [SMILES: *CN(C)CCOCCOC] | 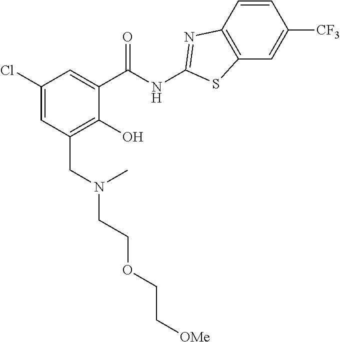 [SMILES: COCCOCCN(C)Cc1cc(Cl)cc(C(=O)Nc2nc3ccc(C(F)(F)F)cc3s2)c1O] |
| | | 24 |
| 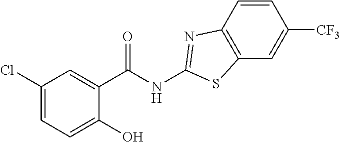 [SMILES: O=C(Nc1nc2ccc(C(F)(F)F)cc2s1)c1cc(Cl)ccc1O] | 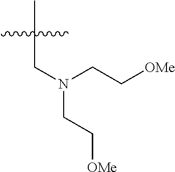 [SMILES: *CN(CCOC)CCOC] | 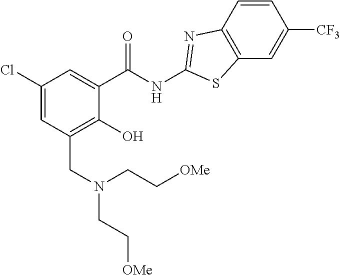 [SMILES: COCCN(CCOC)Cc1cc(Cl)cc(C(=O)Nc2nc3ccc(C(F)(F)F)cc3s2)c1O] |
| | | 25 |
| 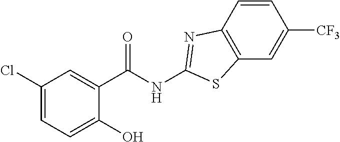 [SMILES: O=C(Nc1nc2ccc(C(F)(F)F)cc2s1)c1cc(Cl)ccc1O] | 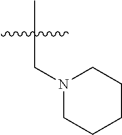 [SMILES: *CN1CCCCC1] | 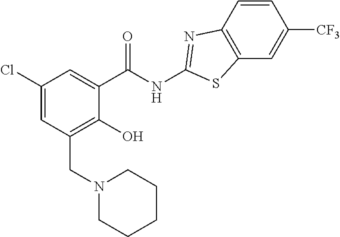 [SMILES: O=C(Nc1nc2ccc(C(F)(F)F)cc2s1)c1cc(Cl)cc(CN2CCCCC2)c1O] |
| | | 26 |
| 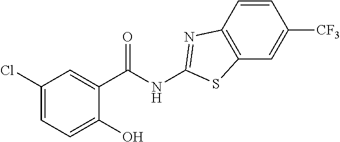 [SMILES: O=C(Nc1nc2ccc(C(F)(F)F)cc2s1)c1cc(Cl)ccc1O] | 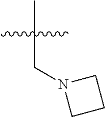 [SMILES: *CN1CCC1] | 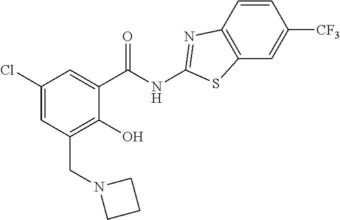 [SMILES: O=C(Nc1nc2ccc(C(F)(F)F)cc2s1)c1cc(Cl)cc(CN2CCC2)c1O] |
| | | 27 |
| 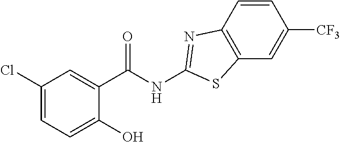 [SMILES: O=C(Nc1nc2ccc(C(F)(F)F)cc2s1)c1cc(Cl)ccc1O] | 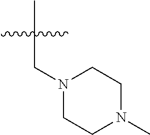 [SMILES: *CN1CCN(C)CC1] | 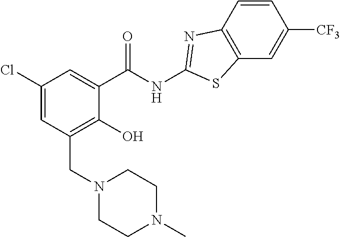 [SMILES: CN1CCN(Cc2cc(Cl)cc(C(=O)Nc3nc4ccc(C(F)(F)F)cc4s3)c2O)CC1] |
| | | 28 |
| 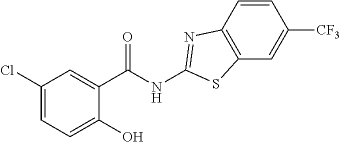 [SMILES: O=C(Nc1nc2ccc(C(F)(F)F)cc2s1)c1cc(Cl)ccc1O] | 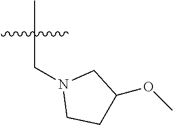 [SMILES: *CN1CCC(OC)C1] | 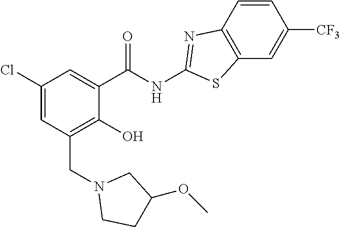 [SMILES: COC1CCN(Cc2cc(Cl)cc(C(=O)Nc3nc4ccc(C(F)(F)F)cc4s3)c2O)C1] |
| | | 32 |
| 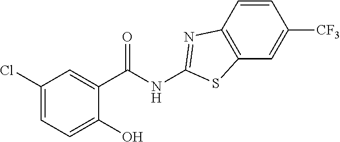 [SMILES: O=C(Nc1nc2ccc(C(F)(F)F)cc2s1)c1cc(Cl)ccc1O] | 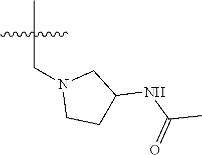 [SMILES: *CN1CCC(NC(C)=O)C1] | 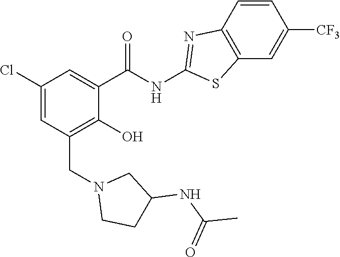 [SMILES: CC(=O)NC1CCN(Cc2cc(Cl)cc(C(=O)Nc3nc4ccc(C(F)(F)F)cc4s3)c2O)C1] |
| | | 33 |
| 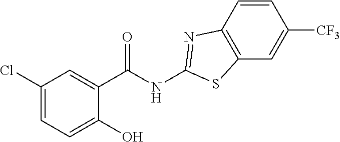 [SMILES: O=C(Nc1nc2ccc(C(F)(F)F)cc2s1)c1cc(Cl)ccc1O] | 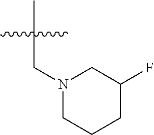 [SMILES: *CN1CCCC(F)C1] | 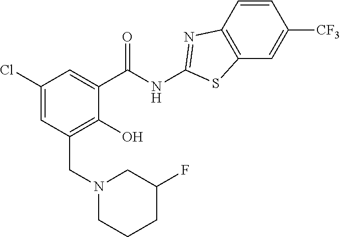 [SMILES: O=C(Nc1nc2ccc(C(F)(F)F)cc2s1)c1cc(Cl)cc(CN2CCCC(F)C2)c1O] |
| | | 58 |
| 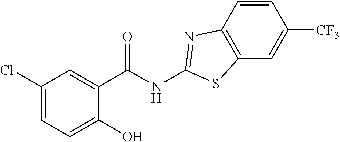 [SMILES: O=C(Nc1nc2ccc(C(F)(F)F)cc2s1)c1cc(Cl)ccc1O] | 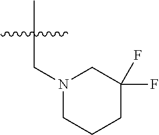 [SMILES: *CN1CCCC(F)(F)C1] | 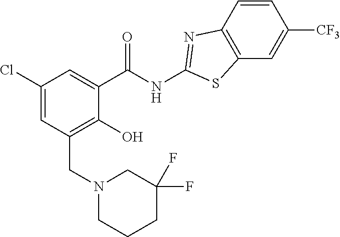 [SMILES: O=C(Nc1nc2ccc(C(F)(F)F)cc2s1)c1cc(Cl)cc(CN2CCCC(F)(F)C2)c1O] |
| | | 59 |
| 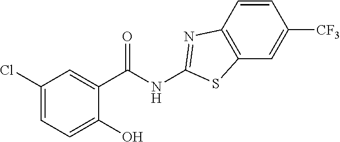 [SMILES: O=C(Nc1nc2ccc(C(F)(F)F)cc2s1)c1cc(Cl)ccc1O] | 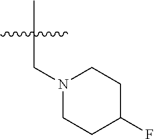 [SMILES: *CN1CCC(F)CC1] | 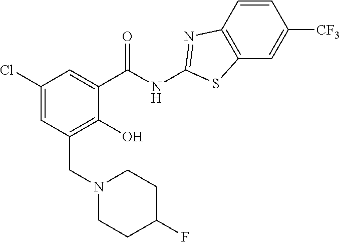 [SMILES: O=C(Nc1nc2ccc(C(F)(F)F)cc2s1)c1cc(Cl)cc(CN2CCC(F)CC2)c1O] |
| | | 60 |
| 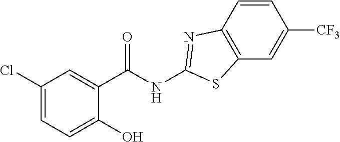 [SMILES: O=C(Nc1nc2ccc(C(F)(F)F)cc2s1)c1cc(Cl)ccc1O] | 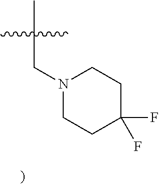 [SMILES: *CN1CCC(F)(F)CC1] | 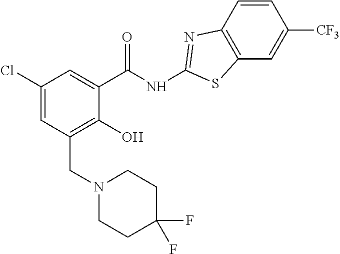 [SMILES: O=C(Nc1nc2ccc(C(F)(F)F)cc2s1)c1cc(Cl)cc(CN2CCC(F)(F)CC2)c1O] |
| | | 62 |
| 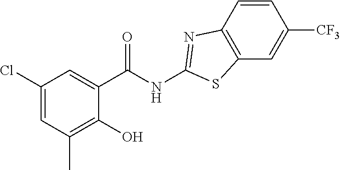 [SMILES: Cc1cc(Cl)cc(C(=O)Nc2nc3ccc(C(F)(F)F)cc3s2)c1O] | 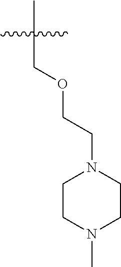 [SMILES: *COCCN1CCN(C)CC1] | 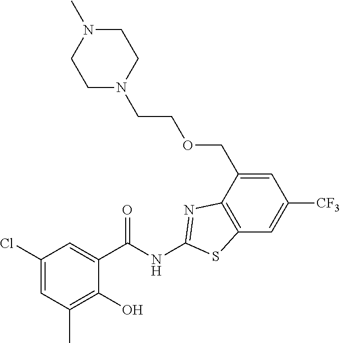 [SMILES: Cc1cc(Cl)cc(C(=O)Nc2nc3c(COCCN4CCN(C)CC4)cc(C(F)(F)F)cc3s2)c1O] |
| | | 75 |
| 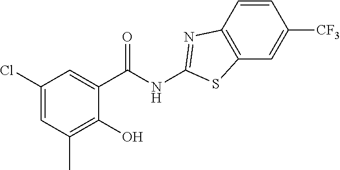 [SMILES: Cc1cc(Cl)cc(C(=O)Nc2nc3ccc(C(F)(F)F)cc3s2)c1O] | 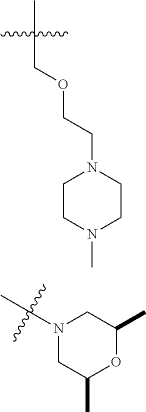 | 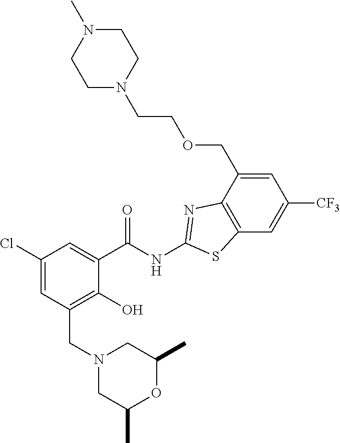 [SMILES: C[C@@H]1CN(Cc2cc(Cl)cc(C(=O)Nc3nc4c(COCCN5CCN(C)CC5)cc(C(F)(F)F)cc4s3)c2O)C[C@H](C)O1] |
| | | 76 |
| 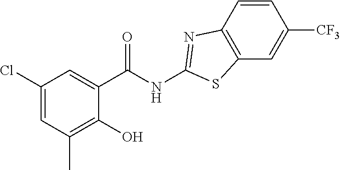 [SMILES: Cc1cc(Cl)cc(C(=O)Nc2nc3ccc(C(F)(F)F)cc3s2)c1O] | 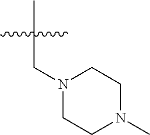 [SMILES: *CN1CCN(C)CC1] | 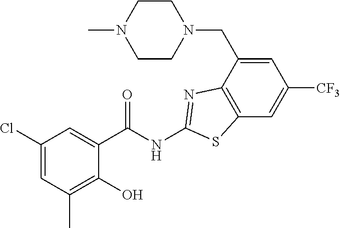 [SMILES: Cc1cc(Cl)cc(C(=O)Nc2nc3c(CN4CCN(C)CC4)cc(C(F)(F)F)cc3s2)c1O] |
| | | 78 |
| 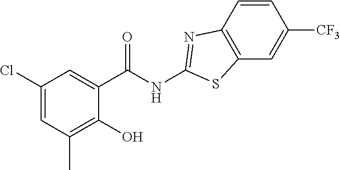 [SMILES: Cc1cc(Cl)cc(C(=O)Nc2nc3ccc(C(F)(F)F)cc3s2)c1O] | 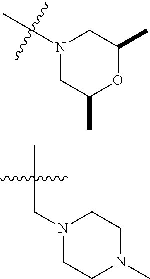 | 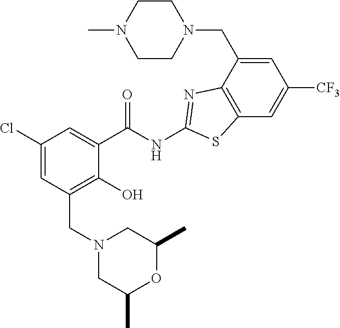 [SMILES: C[C@@H]1CN(Cc2cc(Cl)cc(C(=O)Nc3nc4c(CN5CCN(C)CC5)cc(C(F)(F)F)cc4s3)c2O)C[C@H](C)O1] |
| | | 79 |
| 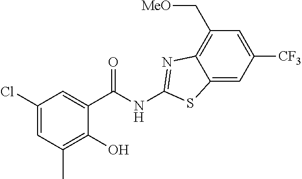 [SMILES: COCc1cc(C(F)(F)F)cc2sc(NC(=O)c3cc(Cl)cc(C)c3O)nc12] | 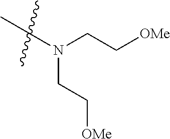 [SMILES: *N(CCOC)CCOC] | 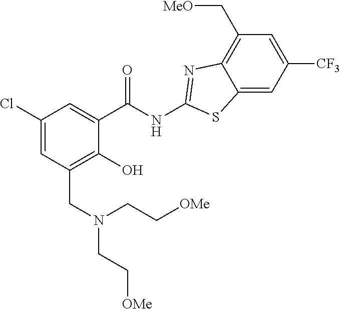 [SMILES: COCCN(CCOC)Cc1cc(Cl)cc(C(=O)Nc2nc3c(COC)cc(C(F)(F)F)cc3s2)c1O] |
| | | 69 |
| 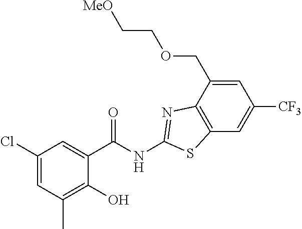 [SMILES: COCCOCc1cc(C(F)(F)F)cc2sc(NC(=O)c3cc(Cl)cc(C)c3O)nc12] | 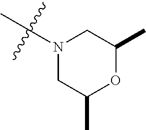 [SMILES: *N1C[C@@H](C)O[C@@H](C)C1] | 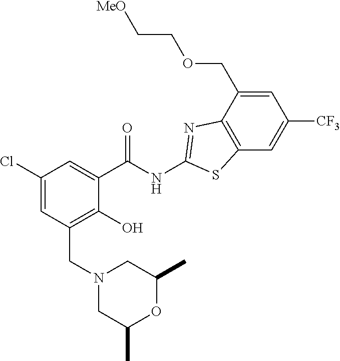 [SMILES: COCCOCc1cc(C(F)(F)F)cc2sc(NC(=O)c3cc(Cl)cc(CN4C[C@@H](C)O[C@@H](C)C4)c3O)nc12] |
| | | 64 |
| 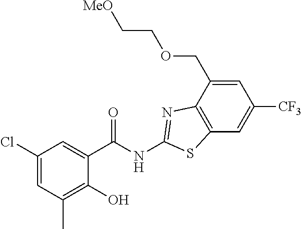 [SMILES: COCCOCc1cc(C(F)(F)F)cc2sc(NC(=O)c3cc(Cl)cc(C)c3O)nc12] | 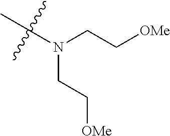 [SMILES: *N(CCOC)CCOC] | 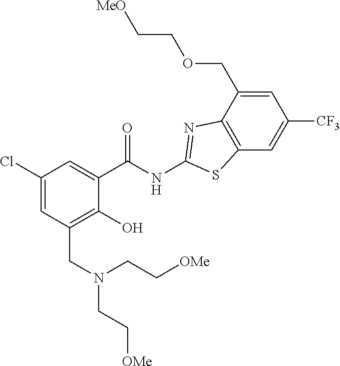 [SMILES: COCCOCc1cc(C(F)(F)F)cc2sc(NC(=O)c3cc(Cl)cc(CN(CCOC)CCOC)c3O)nc12] |
| | | 70 |
| 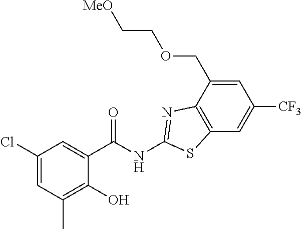 [SMILES: COCCOCc1cc(C(F)(F)F)cc2sc(NC(=O)c3cc(Cl)cc(C)c3O)nc12] | 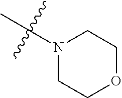 [SMILES: *N1CCOCC1] | 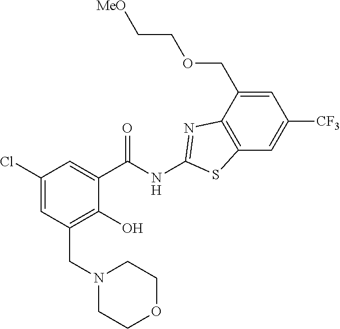 [SMILES: COCCOCc1cc(C(F)(F)F)cc2sc(NC(=O)c3cc(Cl)cc(CN4CCOCC4)c3O)nc12] |
| | | 65 |
| 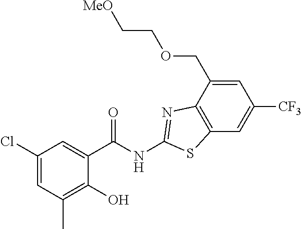 [SMILES: COCCOCc1cc(C(F)(F)F)cc2sc(NC(=O)c3cc(Cl)cc(C)c3O)nc12] | 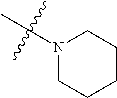 [SMILES: *N1CCCCC1] | 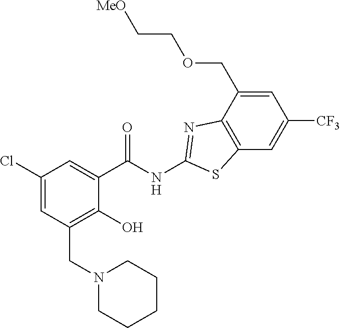 [SMILES: COCCOCc1cc(C(F)(F)F)cc2sc(NC(=O)c3cc(Cl)cc(CN4CCCCC4)c3O)nc12] |
| | | 66 |
| 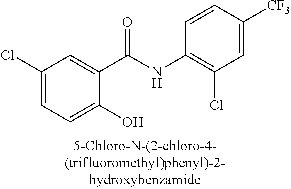 [SMILES: O=C(Nc1ccc(C(F)(F)F)cc1Cl)c1cc(Cl)ccc1O] | 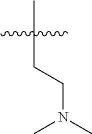 | 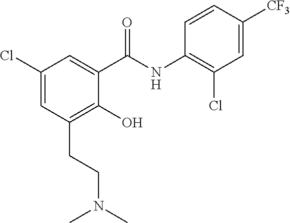 [SMILES: CN(C)CCc1cc(Cl)cc(C(=O)Nc2ccc(C(F)(F)F)cc2Cl)c1O] |
| | | 18 |
| 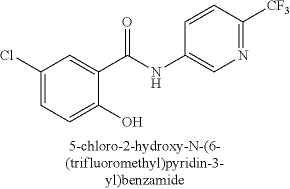 [SMILES: O=C(Nc1ccc(C(F)(F)F)nc1)c1cc(Cl)ccc1O] | 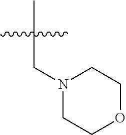 [SMILES: *CN1CCOCC1] | 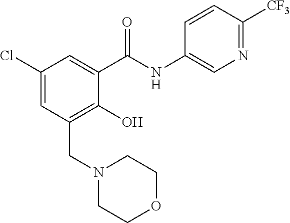 [SMILES: O=C(Nc1ccc(C(F)(F)F)nc1)c1cc(Cl)cc(CN2CCOCC2)c1O] |
| | | 53 |

### Methods of Treatment

In some embodiments, a method of treating a mitochondria-related condition or disorder, in a subject in need thereof, comprises administering to the subject a therapeutically effective amount of a compound or a pharmaceutical composition according to embodiments described herein.

In some embodiments, the mitochondria-related condition or disorder has and one or more underlying causal factors selected from the group consisting of hyperglycemia, abnormal accumulation of lipid in cells, abnormal accumulation of lipid in tissues, abnormal lipid metabolism, insulin resistance, abnormal cell proliferation, abnormal TGF-beta activation, and abnormal fibrosis. In some embodiments, the mitochondria-related condition or disorder has and one or more underlying causal factors selected from the group consisting of hyperglycemia, lipid accumulation, insulin resistance, altered cellular metabolism, fibrosis, aberrant TGF-beta activation, or aberrant cell proliferation.

In some embodiments, the mitochondria-related condition or disorder has and one or more underlying symptoms selected from the group consisting of hyperglycemia, abnormal accumulation of lipid in cells, abnormal accumulation of lipid in tissues, abnormal lipid metabolism, insulin resistance, abnormal cell proliferation, abnormal TGF-beta activation, and abnormal fibrosis. In some embodiments, the mitochondria-related condition or disorder has and one or more underlying causal factors selected from the group consisting of hyperglycemia, lipid accumulation, insulin resistance, altered cellular metabolism, fibrosis, aberrant TGF-beta activation, or aberrant cell proliferation.

In some embodiments, the mitochondria-related condition or disorder is a metabolic disease, cancer, an autoimmune disease, pulmonary fibrosis, a dermatological disorder, an infectious disease or a neurodegenerative disease. In some embodiments, the mitochondria-related condition or disorder is a metabolic disease, cancer, and autoimmune disease, or infectious disease.

In some embodiments, the mitochondria-related condition or disorder is a metabolic disease. In some embodiments the metabolic disease is selected from the group consisting of type 2 diabetes, a disease characterized by insulin resistance or hyperglycemia; obesity or obesity related complications, and a disease characterized by abnormal lipid accumulation.

In some embodiments, a method of treating a metabolic disease or disorder characterized by insulin resistance or by abnormal accumulation of lipid in tissue, or a disease or a disorder in which insulin resistance or abnormal accumulation of lipid in tissue is a symptom, or treating cancer or hyperplasia, in a subject in need thereof, comprises administering to the subject a therapeutically effective amount of a compound or a pharmaceutical composition according to embodiments described herein. In some embodiments, the metabolic disease or disorder is type 2 diabetes, or a disease characterized by insulin resistance or hyperglycemia.

In some embodiments, a metabolic disease or disorder described in any embodiment herein is a complication caused by type 2 diabetes, selected from the group consisting of diabetes-induced cardiovascular diseases, neurodegenerative disorders, atherosclerosis, hypertension, coronary heart diseases, nephropathy, retinopathy, neuropathy, and diabetic heart failure. In some embodiments, the metabolic disease or disorder is obesity or obesity related complications.

In some embodiments, a metabolic disease or disorder described in any embodiment herein is non-alcoholic fatty liver disease (NAFLD), comprising at least one prognosis stage of this disease selected from the group consisting of hepatic steatosis, non-alcoholic steatohepatitis (NASH), cirrhosis, and NAFLD induced hepatocellular carcinoma (HCC). In some embodiments, the metabolic disease or disorder is alcoholic fatty liver disease, or a complication caused by alcoholic fatty liver diseases. In some embodiments, the complication of alcoholic fatty liver disease comprises alcoholic hepatitis, cirrhosis, or a combination thereof.

In some embodiments, the metabolic disease or disorder is dyslipidemia, or a complication caused by dyslipidemia.

In some embodiments, the cancer is a primary cancer selected from the group consisting of hepatocellular carcinoma, colorectal carcinoma, pancreatic cancer, breast cancer, prostate cancer, leukemia, lymphoma, melanoma, ovarian cancer, and lung cancer. In some embodiments, the cancer is a metastatic cancer originated from a primary tumor of other tissue types. In some embodiments, the metastatic sites are selected from the group consisting of liver, lung and the intraperitoneal cavity.

In some embodiments, the compound of embodiments described herein is administered in combination with a second agent indicated for the above-mentioned disorders or diseases, either concomitant with, prior to, or after the administration of the second agent. In some embodiments, the second agent is an anti-diabetic agent selected from the group consisting of metformin, insulin, insulin analogs, sulfonylureas, biguanides, meglitinides, thiazolidinediones, alpha glucosidase inhibitors, GLP-1 agonists, SGLT2 inhibitors and DPP-4 inhibitors. In some embodiments, the second agent is an anti-obesity agent. In some embodiments, the second agent is an anti-nonalcoholic fatty liver disease agent. In some embodiments, the second agent is anti-alcoholic fatty liver disease agent. In some embodiments, the second agent is an anti-dyslipidemia agent.

In some embodiments, a compound of embodiments described herein, is administered in combination with a second anti-non-alcoholic fatty liver disease agent. In some embodiments, a compound of the invention is administered in combination with a second anti-alcoholic fatty liver disease agent. In some embodiments, a compound of the invention is administered in combination with a second anti-dyslipidemia agent.

In some embodiments, the compound may be administered in combination with a second anti-cancer agent or anti-cancer regimen. In some embodiment the second anti-cancer agent s an immunoncological agent. In some embodiments the immunocological agent is selected from the group consisting of an antibody against PD-1/PD-L1, an antibody against other immune check point proteins, CAR-T cells, and other therapeutic immune cells. In some embodiments, the compound may be administered prior to, concomitantly with, or subsequently to administration of the second anti-metabolic disease or anti-cancer agent.

In some embodiments, the subject is a mammalian animal. In some embodiments, the subject is a human. In some embodiments, the compound described herein is used as a veterinarian drug to treat diabetes or a diabetes-associated disease, and the subject is a mammalian animal.

Some embodiments are directed to a method for long-term disease management of a metabolic disease or disorder comprising administering to a subject in need of such long-term management an effective amount of a compound or a pharmaceutical composition described herein. In some embodiments, a method for long-term disease management of a metabolic disease or disorder, or for long-term disease management of cancer, comprises administering to a subject in need of such long-term management an effective amount of a compound or a pharmaceutical composition according to any of the embodiments described herein. In some embodiments, the metabolic disease or disorder is obesity, obesity-related complications, type 2 diabetes, or type 2 diabetes related complications. In some embodiments, the cancer is any primary tumor or metastatic tumor.

In some embodiments, the present disclosure describes the use of a compound according to any of the embodiments described herein in the manufacture of a medicament for treatment of diabetes, obesity, non-alcoholic fatty liver disease, alcoholic fatty liver disease, dyslipidemia, or a disease where insulin resistance or abnormal lipid accumulation in tissue is a symptom, or related disorders or complications, including, but not limited to, hepatic steatosis, non-alcoholic steatohepatitis (NASH), cirrhosis, or NAFLD induced hepatocellular carcinoma (HCC). In some embodiments, the compound of embodiments herein may be used to manufacture a medicament for the treatment of cancer, a disease where cell proliferation (hyperplasia) is a symptom, or cancer or hyperplasia related complications.

Some embodiments herein are directed to a method of treating or preventing a metabolic disease or disorder in a subject in need thereof, comprising administering to the subject a therapeutically effective amount of a compound or pharmaceutical composition described herein.

Some embodiments herein provide for a method of treating and alleviating the symptoms of obesity (characterized by excessive accumulation of lipid in adipocytes), pre-type 2 diabetes (characterized by insulin resistance usually caused by ectopic accumulation of lipid in cells of liver and muscle), type 2 diabetes (characterized by insulin resistance and hyperglycemia), non-alcoholic fatty liver diseases or alcoholic fatty liver disease (characterized by abnormal accumulation of lipid in liver), dyslipidemia (characterized by abnormal lipid deposit in tissue other than adipose), and one or more complications of the above mentioned metabolic disorders, including, but not limited to, hypertension, cardiovascular diseases, nephropathy, and neuropathy comprising administering to the subject a therapeutically effective amount of a compound or pharmaceutical composition described herein. These diseases or disorders may be caused by dietary, environmental, medical and/or genetic factors. The methods described herein may also be used for prevention of the above-mentioned metabolic diseases for a subject with risk factors including, but not limited to, dietary, environmental, medical, and genetic predispositions. In addition, some embodiments provide a method for long-term chronic disease management and longevity management by reducing insulin resistance or reducing glucose levels in the blood.

In some embodiments, the metabolic disease or disorder is type 2 diabetes, or related diseases leading to insulin resistance or hyperglycemia. In some embodiments, the metabolic disease or disorder is obesity or one or more obesity related complications.

In some embodiments, the metabolic disease or disorder is non-alcoholic fatty liver disease, (NAFLD), including nonalcoholic steatohepatitis (NASH) and cirrhosis, or alcoholic fatty liver disease (AFLD). In some embodiments, the metabolic disease or disorder is hepatic steatosis, non-alcoholic steatohepatitis (NASH), cirrhosis, or NAFLD induced hepatocellular carcinoma (HCC).

In some embodiments, the metabolic diseases or disorder is one or more complications of type 2 diabetes including, but not limited to, type 2 diabetes induced hypertension, cardiovascular disease, nephropathy, atherosclerosis, dyslipidemia, retinopathy, neurodegenerative disorders, diabetic heart failure, and neuropathy. In some embodiments, the metabolic disease or disorder is pre-type 2 diabetes. In some embodiments, the metabolic disease or disorder is dyslipidemia.

In some embodiments, the disease to be treated may be a mitochondrial disorder. In some embodiments, the metabolic disorder may be LHON (leber heredity optic neuropathy), MELAS (mitochondrial myopathy, mitochondrial encephalomyopathy, lactic acidosis and stroke-like episodes), MERRF (myoclonic epilepsy and ragged red muscle fiber), Leigh Syndrome, MILS (maternally inherited Leigh Syndrome), NARP (neurogenic muscle weakness, ataxia and retinitis pigmentosa), FBSN (familial bilateral striatal necrosis), or KSS (Kearns Sayre Syndrome).

Some embodiments are directed to a method of treating or preventing cancer in a subject in need thereof, comprising administering to the subject a therapeutically effective amount of a compound or pharmaceutical composition described herein. In some embodiments, the cancer may be primary cancer or metastatic cancer. In some embodiments, the cancer is primary cancer including but not limited to hepatocellular carcinoma, colorectal carcinoma, pancreatic cancer, breast cancer, prostate cancer, leukemia, lymphoma, melanoma, ovarian cancer, lung cancer. In some embodiments, the cancer is metastatic liver cancer originated from the primary tumor of other tissue types. In some embodiments, the cancer is metastatic lung cancer originated from the primary tumor of other tissue types. In some embodiments, the cancer is metastatic cancer to other sites including intraperitoneal cavity.

Some embodiments are directed to a method of treating or preventing autoimmune diseases in a subject in need thereof, comprising administering to the subject a therapeutically effective amount of a compound or pharmaceutical composition described herein. In some embodiments, the autoimmune disease is celiac disease, diabetes mellitus type 1, Graves' disease, inflammatory bowel disease, multiple sclerosis, psoriasis, rheumatoid arthritis, and systemic lupus erythematosus.

Some embodiments are directed to a method of treating or preventing a dermatological disorder in a subject in need thereof, comprising administering to the subject a therapeutically effective amount of a compound or pharmaceutical composition described herein. In some embodiments, the dermatological disorder is eczema, dyshidrotic eczema, seborrheic eczema psoriasis, rosacea, dermatitis and atopic dermatitis.

Some embodiments are directed to a method of treating or preventing an infectious disease of non-viral parasites in a subject in need thereof, comprising administering to the subject a therapeutically effective amount of a compound or pharmaceutical composition described herein. In some embodiments, the infectious disease is a viral infections. In some embodiments the viral infection is an envelope viral infection. In some embodiments the viral infection is selected from the group consisting of SARS-CoV-2, a corana virus infection and an Ebola viral infection.

In some embodiments, the disease to be treated may be a heart disorder comprising administering to the subject a therapeutically effective amount of a compound or pharmaceutical composition described herein. In some embodiments, the heart disorder may be hypertension or cardiovascular disease. In some embodiments, the disease to be treated may be a central nervous system (CNS) disease. In some embodiments, the CNS disease may be stroke, Alzheimer's, Parkinson's, Huntington's, or ALS (amyotropic lateral sclerosis).

In some embodiments, the disease to be treated may be a disorder associated with increased ROS (reactive oxygen species) production comprising administering to the subject a therapeutically effective amount of a compound or pharmaceutical composition described herein. Increased ROS has been associated with aging, Alzheimer's disease, Parkinson's disease, Huntington's disease, ALS (amyotropic lateral sclerosis), mitochondrial diseases, and various cancers.

The compounds and pharmaceutical compositions described herein may be administered by oral, parenteral (e.g., intramuscular, intraperitoneal, intravenous, ICV, intracisternal injection or infusion, subcutaneous injection, or implant), by inhalation spray, ophthalmic, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. The compounds and pharmaceutical compositions described herein may also be formulated as a controlled-release formulation.

The compounds described herein may be administered topically and can be formulated into a variety of topically administrable pharmaceutical compositions comprising an active ingredient and a dermatologically acceptable base and/or an ophthalmically acceptable base. Such pharmaceutical compositions can be formulated, e.g., as solutions, suspensions, spray, lotions, gels, pastes, medicated sticks, balms, shampoos, soap bars, liquid soaps, creams or ointments. In one embodiment, the pharmaceutical composition is the form of an ointment that can be applied in or around the eye of a mammal, including a human.

In some embodiments, a dermatologically and/or ophthalmically acceptable base includes a pharmaceutically acceptable ointment base. Examples of suitable ointment bases include, but are not limited to oleaginous ointment bases such as petrolatum (e.g., liquid petrolatum or white petrolatum), plastibase, hard paraffin, white soft paraffin, yellow soft paraffin, liquid paraffin, emulsifying wax, microcrystalline wax, white bees wax, yellow bees wax, carnauba wax, wool wax (wool fat), mineral oil, olive oil, purified lanolin, anhydrous lanolin, and water soluble ointment bases such as polyethylene glycol (e.g., polyethylene glycol 400 or polyethylene glycol 3350), propylene glycol, polyoxyethylene, polyoxypropylene, or any combinations thereof.

In some embodiments, a dermatologically and/or ophthalmically acceptable base includes one or more polymers as suspending agents. Useful polymers include, but are not limited to, water-soluble polymers such as cellulosic polymers, e.g., hydroxypropyl methylcellulose, and water-insoluble polymers such as cross-linked carboxyl-containing polymers. A dermatologically and/or ophthalmically acceptable base can also include a dermatologically and/or ophthalmically acceptable mucoadhesive polymer, e.g., carboxymethylcellulose, carbomer (acrylic acid polymer), carbopol (copolymers or acrylic acid crosslinked with a polyakenyl polyether), poly(methylmethacrylate), polyacrylamide, polycarbophil, acrylic acid/butyl acrylate copolymer, sodium alginate, or dextran.

In some embodiments, a dermatologically and/or ophthalmically acceptable base includes one or more viscosity enhancing agents. Examples of suitable viscosity enhancing agents include, but are not limited to, methyl cellulose, xanthan gum, gum tragacanth, carboxymethyl cellulose, silica, silicone, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, acacia, corn starch, gelatin, or combinations thereof.

In some embodiments, a dermatologically and/or ophthalmically acceptable base includes one or more dermatologically and/or ophthalmically acceptable pH adjusting agents or buffering agents, including, but not limited to, acids such as acetic, boric, citric, lactic, phosphoric and hydrochloric acids; bases such as sodium hydroxide, sodium phosphate, sodium borate, sodium citrate, sodium acetate, sodium, lactate and tris-hydroxymethylaminomethane; and buffers such as citrate/dextrose, sodium bicarbonate and ammonium chloride. Such acids, bases and buffers are included in an amount required to maintain pH of the composition in a dermatologically and/or ophthalmically acceptable range.

In some embodiments, a dermatologically and/or ophthalmically acceptable base includes one or more dermatologically and/or ophthalmically acceptable salts in an amount required to bring osmolality of the composition into a dermatologically and/or ophthalmically acceptable range. Such salts include, but are not limited to, those having sodium, potassium or ammonium cations and chloride, citrate, ascorbate, borate, phosphate, bicarbonate, sulfate, thiosulfate or bisulfite anions; specific salts include, e.g., sodium chloride, potassium chloride, sodium thiosulfate, sodium bisulfite, and ammonium sulfate.

In some embodiments, a dermatologically and/or ophthalmically acceptable base includes one or more dermatologically and/or ophthalmically acceptable preservatives to inhibit microbial activity. Suitable preservatives include, but are not limited to, mercury-containing substances such as merfen and thiomersal; stabilized chlorine dioxide; and quaternary ammonium compounds such as benzalkonium chloride, cetyltrimethylammonium bromide and cetylpyridinium chloride.

In further embodiments, a dermatologically and/or ophthalmically acceptable base includes one or more dermatologically and/or ophthalmically acceptable surfactants to enhance physical stability, or for other purposes. Suitable nonionic surfactants include isohexadecane, cyclomethicone, copolymers of ethylene glycol and propylene glycol, polyoxyethylerie fatty acid glycerides and vegetable oils, e.g., polyoxyethylene (60) hydrogenated castor oil; and polyoxyethylerie alkylethers and alkylphenyl ethers, e.g., octoxynol 10, octoxynol 40.

In further embodiments, a dermatologically and/or ophthalmically acceptable base includes one or more dermatologically and/or ophthalmically acceptable penetration enhancers to enhance physical stability, or for other purposes. Penetration enhancers are substances which enhance passage of topically-applied compounds into the stratum, corneum of the skin and therefrom into the epidermis and dermis. Examples include, but are not limited to: dimethyl isosorbide, ethoxydiglycol, 1-dodecylazacycloheptan-2-one, propylene glycol, oleyl alcohol, polyoxyethylene ester, sorbitan mono-9-octadecenoate, poly(oxy-1,2-ethanediyl) and derivatives thereof, ethanol, glyceryl monoethyl ether, monoglycerides, isopropylmyristate, lauryl alcohol, lauric acid, lauryl lactate, terpinol, menthol, D-limonene, beta-cyclodextrin, DMSO (dimethyl sulfoxide), polysorbates, fatty acids (e.g., oleic), bile salts, N-methylpyrrolidone, polyglycosylated glycerides, 1-dodecylazacycloheptan-2-one (Azone®), Cyclopentadecalactone (CPE-215®), Alkyl-2-(N,N-disubstituted amino)-alkanoate ester (NexAct®), 2-(n-nonyl)-1,3-dioxolane (DEPA®), and penetration enhancers shown for example in U.S. Pat. Nos. 3,909,816; 4,405,616; 4,801,586; 4,861,764; 4,886,783; 4,983,396; 5,118,845; 5,196,410, 8,486,374 and 8,741,265, each of which is hereby expressly incorporated herein by reference in its entirety.

In further embodiments, a dermatologically and/or ophthalmically acceptable base includes one or more dermatologically and/or ophthalmically acceptable permability enhancers to enhance physical stability, or for other purposes. A variety of classes of compounds may serve as suitable permeability enhancers according to the invention. A first category includes fatty acids and salts and esters thereof, including mono-, di-, and triglycerides. Medium chain length fatty acids, especially C8 and C10 acids, and their salts and esters are particularly useful. Suitable specific examples include sodium caprylate, sodium caprate, CAPMUL® glycerides (available from Abitec of Columbus, Ohio), LABRASOL® glycerides (PEG-8 caprylic/capric glycerides, available from Gattefosse SAS of Saint Priest, Cedex, France), GELUCIRE® 44/14 (PEG-32 glyceryl laurate EP, available from Gattefosse), other glycerides & fatty acid esters, CREMOPHOR® (BASF, Ludwigshafen, Germany), D-α-tocopheryl polyethylene glycol 1000 succinate, vegetable oils, polyoxylglycerides, and medium chain mono- and diacylglycerides.

One example of this class, CAPMUL® MCM L8 (glycerol monocaprylate) (available from Abitec of Columbus, Ohio), is composed of mono- and diglycerides of medium chain fatty acids (mainly caprylic, with some capric) and 7% maximum free glycerol. It contains at least 44% alpha monoglycerides (as caprylate).

Other examples of this class of enhancers include GATTEFOSSE compositions 61A through 61H which are proprietary to Gattefosse SAS, but generally are composed of mixtures containing one or more of medium chain mono-, di-, or triglycerides, polysorbate derivatives, polyoxyl castor oil derivatives, polyethylene glycol derivatives including polyethylene glycol glycerides, polyoxyl ethers, vegetable oils, glycerin, and similar GRAS (generally regarded as safe) lipidic components in varying amounts. These components are part of individual commercial products such as CAPRYOL™ 90, CAPRYOL™ PGMC, LAUROGLYCOL™ 90, GELUCIRE® 44/14, Plurol Oleique CC497, LABRASOL®, LABRAFIL® M1944CS (apricot kernel oil PEG-6 esters), Transcutol HP, Peceol, and Maisine 35-1, all of which are available from Gattefosse SAS.

While not falling directly within this class, glycerol itself has been found to impart excellent permeability enhancement, particularly for neuraminidase inhibitors. This result was not anticipated as glycerol is not considered a permeability enhancer.

A second category of enhancers includes surfactants having a steroidal structure, such as bile acid salts. Examples of suitable compounds include sodium cholate, sodium deoxycholate, glycocholate, glycoursodeoxycholate, taurocholate, taurodeoxycholate, and steroid detergents/bile salts. Other surfactants may also be suitable permeability enhancers, including cationic, anionic, and nonionic surfactants. Examples include polysorbate 80, hexadecyldimethylbenzylammonium chloride, N-hexadecylpyridinium bromide, dodecyltrimethylammonium bromide, hexadecyltrimethylammonium bromide, tetradecyl-β-D-maltoside, octylglucoside, glycyrrhetinic acid, 3-(N,N-dimethylpalmitylammonio)propane-sulfonate, and sodium lauryl sulfate.

Cyclodextrins may also be used as suitable enhancers. Examples include β-cyclodextrin, hydroxypropyl-β-cyclodextrin, γ-cyclodextrin, and hydroxypropyl-γ-cyclodextrin.

A variety of other compounds may also be used as enhancers. Examples include sodium salicylate, ethylenediamine tetraacetic acid (EDTA), citric acid, chitosan & chitosan derivatives, N-trimethyl chitosan chloride, monocarboxymethyl-chitosan, palmitoyl carnitine chloride, acyl carnitines, ethylene glycol tetraacetic acid (EGTA), 3-alkylamido-2-alkoxypropyl-phosphocholine derivatives, alkanoylcholines, N-acetylated amino acids (based on α- and non-α-amino acids), mucoadhesive polymers, phospholipids, piperine, 1-methylpiperazine, α-amino acids, and mineral oil.

Thus a wide variety of enhancer compounds may be selected from the group consisting of fatty acids, fatty acid esters, fatty acid salts, glycerol, surfactants, cyclodextrins, sodium salicylate, ethylenediamine tetraacetic acid, citric acid, chitosan, chitosan derivatives, N-trimethyl chitosan chloride, monocarboxymethyl-chitosan, palmitoyl carnitine chloride, acyl carnitines, ethylene glycol tetraacetic acid, 3-alkylamido-2-alkoxypropyl-phosphocholine derivatives, alkanoylcholines, N-acetylated amino acids, mucoadhesive polymers, phospholipids, piperine, 1-methylpiperazine, α-amino acids, and mineral oil.

The permeability enhancer and the polar agent may be mixed in any proportion so long as there is provided a therapeutically effective amount of the polar agent and a permeability-enhancing amount of the enhancer compound. Enhancement in dermal bioavailability of topically administered polar agents can depend on the nature and concentration of the enhancer compound with which the agent is formulated. It is thus contemplated that the required therapeutic amount may be contained in a single dosage form or divided between one or more dosages intended for application at the same time or in sequence.

The permeability enhancers act relatively independently of the concentration of polar agent. Differing permeability enhancers can reach either optimal or maximum enhancement over a wide concentration range depending on their particular inherent enhancement potential. Often, enhancers have a non-linear dose response relationship between concentration of enhancer present and amount of increased polar agent absorption. The amount of enhancer to be utilized in an oral dosage form with a polar agent is initially based upon the enhancement properties observed in Caco-2 cell assays at varying fixed enhancer concentrations. Based upon those results, an effective in vivo amount of enhancer compound for a human formulation can be estimated, demonstrated and optimized without undue experimentation using methods well known to those skilled in the formulation art, to achieve a desired pharmacokinetic in vivo profile.

In formulating the composition of this invention, it will be apparent to those skilled in the formulation art that more effective enhancer compounds would require less polar agent than less effective permeability enhancers to achieve a target pharmacokinetic profile. Given those considerations and variations, the amount of enhancer may be at least about 0.1 wt % of the combined weight of enhancer and polar agent, more preferably at least about 50 wt %, and more preferably at least 70 wt % of the combined weight of enhancer and polar agent. The amount is preferably at most 95 wt %, more preferably at most 80 wt %, and more preferably at most 75 wt % of the combined weight of the enhancer and polar agent. Thus, as shown in the examples, a typical dosage form may contain a wide range of concentrations of enhancer compounds depending on the compound itself and its efficacy in enhancing the permeability of polar agents following oral administration. Concentrations as low as 0.001% by weight up to 20% have been demonstrated to be effective in enhancement of the permeability of polar agents.

In yet other embodiments, a dermatologically and/or ophthalmically acceptable base includes one or more antioxidants to enhance chemical stability where required. Suitable antioxidants include, by way of example only, butylated hydroxytoluene (BHT), sodium ascorbate, ascorbic acid, sodium metabisulfite, and tocopherol. In certain embodiments, antioxidants enhance chemical stability where required.

In addition to those enumerated above, any other surfactant, moisturizer, gelling agent, preservative, colorant or pigment, antioxidant, radical scavenger, emulsifier, humectant, pH modifier, chelating agent, or other dermatologically acceptable excipient commonly known to those of ordinary skill in the art as useful in topical compositions is contemplated as useful in the compositions described herein. Further, any non-toxic, inert, and effective topical carrier may be used to formulate the compositions described herein.

Well-known carriers used to formulate other topical therapeutic compositions for administration to humans will be useful in these compositions. Examples of such components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, http://www.accessdata.fda.gov/scripts/cder/iig/index.cfm, the contents of which are hereby incorporated by reference in their entirety. Examples of such useful pharmaceutically acceptable excipients, carriers and diluents include distilled water, physiological saline, Ringer's solution, dextrose solution, Hank's solution, and DMSO, which are among those preferred for use herein.

These additional other inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

The composition may be used immediately or stored for later use in any type of container known to one of skill in the art such as, for example, pouch, jar, bottle, tube, ampule and pre-filled syringe. Finally, the composition may be sterilized by any method known to one of skill in the art such as, for example, γ radiation.

The compounds and pharmaceutical compositions described herein may be administered at prophylactically effective dosage levels to prevent the above-recited conditions and disorders, as well as to prevent other conditions and disorders characterized by insulin resistance or hyperglycemia.

The pharmaceutical compositions and compounds of embodiments herein can be administered in a wide range of dosage-forms including, for example, solid dosage forms and liquid dosage forms. Solid dosage forms may include powders, tablets, pills, capsules, suppositories, or dispersible granules. A solid carrier can be one or more substances that function as a diluting agent, flavor additive, solvent, lubricant, suspension agent, binder, preservative, tablet-disintegrating substance or encapsulating material. In powdered form, the carrier may be a finely pulverized solid including lactose, hydroxypropylmethylcellulose and PVP, mixed with an appropriate amount of the active ingredient. Appropriate carriers for powder and tablet forms include for example magnesium carbonate, magnesium stearate, talc, sugar, lactose, pectin, dextrin, stiffeners, gelatins, tragacanth, methylcellulose, and sodium carboxymethylcellulose.

Liquid dosage forms include for example solutions, suspensions, and emulsions. Also included are pharmaceutical compositions in solid form that are meant to be converted to liquid form shortly prior to consumption. These forms may include, in addition to the active ingredients, artificial colors, flavors, stabilizers, buffers, natural or artificial sweeteners, dispersing agents, thickeners, dissolving agents and the like.

Solutions or mixtures may be administered directly to the nasal cavity using conventional means, such as drops or sprays. The pharmaceutical composition may be produced in individual or multi-dose forms. Multi-dose forms would include a dropper, pipette or atomizer that delivers a predetermined volume of the pharmaceutical composition. The pharmaceutical compositions and compounds of embodiments herein may be provided in individual dosage units that contain a suitable amount of the active ingredient. The individual doses may be provided in a package, or as a kit that includes a measuring device, e.g., a device for measuring oral or injectable dosages (i.e., a measuring cup, needle, or syringe). The kit can also include, other materials such buffers, diluents, filters, and package inserts with instructions for use. A label may be present on the on the kit to indicate that the pharmaceutical composition is used for a specific therapy, and may also indicate directions for use.

If desired, the pharmaceutical compositions of the present invention may further comprise one or more additional active agents. Where it is appropriate, any of the active agents may be administered in the form of the compound per se, and/or in the form of a salt, polymorph, ester, amide, prodrug, derivative, or the like, provided the salt, polymorph, ester, amide, prodrug or derivative is suitable pharmacologically. Where it is appropriate, salts, esters, amides, prodrugs and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For any active agents that may exist in enantiomeric forms, the active agent may be incorporated into the present pharmaceutical compositions either as the racemate or in enantiomerically enriched form.

The dosage of the active compound(s) being administered will depend on the condition being treated, the particular compound, and other clinical factors such as age, sex, weight, and health of the subject being treated, the route of administration of the compound(s), and the type of pharmaceutical composition being administered (tablet, gel cap, capsule, solution, suspension, inhaler, aerosol, elixir, lozenge, injection, patch, ointment, cream, etc.). It is to be understood that the present disclosure has application for both human and animal use. The amount of the compound, or an active salt or derivative thereof, required for use in treatment will be ultimately at the discretion of the attendant physician or clinician.

As described above, the compounds of the invention are useful for the prevention, treatment, control, amelioration, or reduction of risk of the diseases, disorders and conditions noted herein. The dosage of the compound as an active ingredient in the pharmaceutical compositions of this invention may be varied so that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.001 to 100 mg/kg. of body weight daily are administered to the patient, e.g., humans and elderly humans. The therapeutically effective amount will generally be about 0.5 mg to 10 g per patient per day which may be administered in single or multiple doses. In some embodiments the therapeutically effective amount is between a lower limit of 0.5 mg, 10 mg, 1 mg, 500.0 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, and 10000 mg; and an upper limit of 10000 mg, 9500 mg, 9000 mg, 8500 mg, 8000 mg, 7500 mg, 7000 mg, 6500 mg, 6000 mg, 5500 mg, 5000 mg, 4500 mg, 4000 mg, 3500 mg, 3000 mg, 2500 mg, 2000 mg, 1500 mg, 1000 mg, 500.0 mg, 100 mg, 10 mg and 0.5 mg. In some embodiments, the therapeutically effective amount will be about 0.5 mg to 2500 mg per patient per day; in some embodiments about 0.5 mg to 200 mg per patient per day; in some embodiments about 0.5 mg to 500 mg per patient per day; in some embodiments about 0.5 mg to 1000 mg per patient per day; and in yet some other embodiments about 5 mg to 50 mg per patient per day. Pharmaceutical compositions of the present invention may be provided in a solid dosage formulation such as comprising about 0.5 mg to 500 mg active ingredient, or comprising about 1 mg to 250 mg active ingredient. The pharmaceutical composition may be provided in a solid dosage formulation comprising for example about 1 mg, 5 mg, 10 mg, 25 mg, 50 mg, 100 mg, 200 mg, 250 mg, 500 mg or 1000 mg of active ingredient. For oral administration, the pharmaceutical compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, 1000 and 2000 milligrams of the active ingredient for the symptomatic adjustment of the dosage to the patient to be treated. The compounds may be administered on a regimen of 1 to 4 times per day, such as once, twice, three times or four times per day.

### Definitions

Molecular terms, when used in this application, have their common meaning unless otherwise specified.

The articles “a” and “an” as used herein mean “one or more” or “at least one,” unless otherwise indicated. That is, reference to any element of the present invention by the indefinite article “a” or “an” does not exclude the possibility that more than one of the element is present.

The term “acylamino” denotes a nitrogen radical adjacent to an acyl group.

As used herein, the term “alkyl” is intended to include both branched and straight-chain saturated aliphatic hydrocarbon groups having the specified number of carbon atoms. For example, “C₁-C₁₇-alkyl” or “C₁₋₁₇ alkyl” (or alkylene), is intended to include C₁, C₂, C₃, C₄, C₅, C₆, C₇, C₈, C₉, C₁₀, C₁₁, C₁₂, C₁₃, C₁₄, C₁₅, C₁₆, and C₁₇ alkyl groups. Additionally, for example, “C₁-C₆ alkyl” or “C₁₋₆ alkyl” denotes alkyl having 1 to 6 carbon atoms. Alkyl group can be unsubstituted or substituted with at least one hydrogen being replaced by another chemical group. In some embodiments the one of more hydrogen atoms is replaced by a chemical group selected from hydroxyl, and dimethylamino. Examples of alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl). Examples of substituted alkyl includes, but is not limited to, —CH₂N(CH₃)₂, —CH₂CH₂N(CH₃)₂, and —CH₂CH₂CH₂N(CH₃)₂.

“Alkenyl” is intended to include hydrocarbon chains of either straight or branched configuration having the specified number of carbon atoms and one or more, preferably one to three, carbon-carbon double bonds that may occur in any stable point along the chain. For example, “C₂-C₆ alkenyl” or “C₂₋₆ alkenyl” (or alkenylene), is intended to include C₂, C₃, C₄, C₅, and C₆ alkenyl groups. The term “C₂₋₁₇ alkenyl”, is intended to include C₂, C₃, C₄, C₅, C₆, C₇, C₈, C₉, C₁₀, C₁₁, C₁₂, C₁₃, C₁₄, C₁₅, C₁₆, and C₁₇ alkenyl groups. Examples of alkenyl include, but are not limited to, ethenyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, and 4-methyl-3-pentenyl.

“Alkynyl” is intended to include hydrocarbon chains of either straight or branched configuration having one or more, preferably one to three, carbon-carbon triple bonds that may occur in any stable point along the chain. For example, “C₂-C₆ alkynyl” is intended to include C₂, C₃, C₄, C₅, and C₆ alkynyl groups; such as ethynyl, propynyl, butynyl, pentynyl, and hexynyl.

The term “alkoxy” or “alkyloxy” refers to an —O-alkyl group. “C₁-C₆ alkoxy” or “C₁₋₆ alkoxy” (or alkyloxy), is intended to include C₁, C₂, C₃, C₄, C₅, and C₆, alkoxy groups. Examples of alkoxy groups include, but are not limited to, methoxy, ethoxy, propoxy (e.g., n-propoxy and isopropoxy), and t-butoxy.

“Aryl” groups refer to monocyclic or polycyclic aromatic hydrocarbons, including, for example, thiazolyl, phenyl, and naphthyl. “C₆-C₁₀ aryl” or “C₆₋₁₀ aryl” refers to phenyl and naphthyl. Unless otherwise specified, “aryl”, “C₆-C₁₀ aryl,” “C₆₋₁₀ aryl,” or “aromatic residue” may be unsubstituted or substituted with 1 to 5 groups selected from —OH, —OCH₃, —Cl, —F, —Br, —I, —CN, —NO₂, —NH₂, —NH(CH₃), —N(CH₃)₂, —CF₃, —OCF₃, —C(O)CH₃, —SCH₃, —S(O)CH₃, —S(O)₂CH₃, —CH₃, —CH₂CH₃, —CO₂H, and —CO₂CH₃.

The term “benzyl,” as used herein, refers to a methyl group on which one of the hydrogen atoms is replaced by a phenyl group, wherein said phenyl group may optionally be substituted by one to five, preferably one to three, substituents independently selected from methyl, trifluoromethyl (—CF₃), hydroxyl (—OH), methoxy (—OCH₃), halogen, cyano (—CN), nitro (—NO₂), —CO₂Me, —CO₂Et, and —CO₂H. Representative examples of benzyl group include, but are not limited to, PhCH₂—, 4-MeO—C₆H₄CH₂—, 2,4,6-tri-methyl-C₆H₂CH₂—, and 3,4-di-Cl—C₆H₃CH₂—.

The term “carboxyamido” denotes a carbonyl radical adjacent to an amino group.

A “compound,” as used herein, refers to any type of substance or agent that is commonly considered a drug, or a candidate for use as a drug, as well as combinations and mixtures of the above. When referring to a compound of the invention, and unless otherwise specified, the term “compound” is intended to encompass not only the specified molecular entity but also its pharmaceutically acceptable, pharmacologically active analogs, including, but not limited to, salts, polymorphs, esters, amides, prodrugs, adducts, conjugates, active metabolites, and the like, where such modifications to the molecular entity are appropriate.

A “conventional mitochondrial uncoupler” as used herein describes a mitochondrial uncoupler that has properties that increase OCR and decreased MMP, and the concentrations for increasing OCR and dissipating MMP correlate.

As used herein, a “derivative” of a compound refers to a chemical compound that may be produced from another compound of similar structure in one or more steps. Non-limiting examples include replacement of H by an alkyl, acyl, or amino group.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

The terms “formula” and “structure” are used interchangeably herein.

The term “halo” or “halogen” refers to fluoro, chloro, bromo, and iodo. “Haloalkyl” is intended to include both branched and straight-chain saturated aliphatic hydrocarbon groups having the specified number of carbon atoms, substituted with one or more halogens. Examples of haloalkyl include, but are not limited to, fluoromethyl, difluoromethyl, trifluoromethyl, and trichloromethyl.

As used herein, the term “heteroaryl” is intended to mean stable monocyclic and polycyclic aromatic hydrocarbons that include at least one heteroatom ring member, such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl, and benzodioxane. Unless otherwise specified, heteroaryl groups may be unsubstituted or substituted with 1 to 5 groups selected from —OH, —OCH₃, —Cl, —F, —Br, —I, —CN, —NO₂, —NH₂, —NH(CH₃), —N(CH₃)₂, —CF₃, —OCF₃, —C(O)CH₃, —SCH₃, —S(O)CH₃, —S(O)₂CH₃, —CH₃, —CH₂CH₃, —CO₂H, and —CO₂CH₃. The nitrogen atom is substituted or unsubstituted (i.e., N or NR wherein R is H or another substituent, if defined). The nitrogen and sulfur heteroatoms may optionally be oxidized (i.e., N→0 and S(O)ₚ, wherein p is 0, 1 or 2).

The term “heterocyclyl,” “heterocyclic” or “heterocyclyl ring” is defined as a saturated or partially unsaturated ring containing one to four hetero atoms or hetero groups selected from O, N, NH, —N(R^{z})—, —S(O)— or —S(O)₂—, wherein R^{z} is selected from alkyl, alkenyl, alkynyl, aryl, heteroaryl, cycloalkyl, optionally substituted heterocyclyl, in a single or fused heterocyclic ring system having from three to twelve ring members. In a preferred embodiment, a heterocyclyl is a ring system having three to seven ring members Examples of a heterocyclyl group include, without limitation, azetindinyl, pyrrolidinyl, piperidinyl, piperazinyl, oxopiperazinyl, morpholinyl, dioxothiomorphylinyl, tetrahydrofuranyl and azabicyclo[3.2.1]octanyl. Unless otherwise specified, heteroaryl groups may be unsubstituted or substituted with at least one groups selected from oxo, cyano, hydroxyl, alkoxy, -acylamino, carboxyamido, —SO₂CH₃, —CF₃, C₁-C₆ alkyl, halo, and acyl.

The term “infectious disease” as described herein referes to a bacterial infection, or viral infection. Infectious diseases do not include infections caused by parasitic organism. In some embodiments the viral infection is an envelope virus. Examples of envelope viruses include SARS-CoV-2, corana viruses and Ebola viruses.

The term “mitochondria-related condition or disorder” is defined as pathologic conditions caused by mitochondrial malfunction as reviewed and summarized in: A Mitochondrial Paradigm of Metabolic and Degenerative Diseases, Aging, and Cancer: A Dawn for Evolutionary Medicine, Annu Rev Genet. 2005; 39: 359, The rise of mitochondria in medicine, Mitochondrion 2016, 30:105-16, and Is Mitochondrial Dysfunction a Common Root of Noncommunicable Chronic Diseases? Endocrine Reviews 2020,41(491-517), all of which are incorporated herein by reference. These conditions include, but are not limited to: genetic mitochondrial diseases, various types of cancer, autisim, neurodegenerative diseases, neuromuscular diseases, immunological diseases, metabolic diseases, aging, and aging-related noncommunicable chronic diseases.

An “MMP-retaining compound” or a “mitochondrial membrane potential-retaining compound” is defined as a mitochondrial uncoupler that effectively increases OCR without significantly reducing MMP.

The term, “mitochondrial uncoupling”, also referred to as “uncoupling”, refers to the process whereby protons enter the mitochondrial matrix via a pathway independent of ATP synthase and thereby uncouple nutrient oxidation from ATP production. This process can be pharmacologically induced by small molecule mitochondrial protonophores, which directly shuttle protons across the mitochondrial inner membrane into the matrix. The primary pathway for energy production in aerobic cells involves the oxidation of nutrients (including fats, carbohydrates, and amino acids) in mitochondria, which promotes the efflux of protons out of the mitochondrial matrix. This process creates a pH and electrochemical gradient across the mitochondrial inner membrane. Protons normally re-enter the mitochondrial matrix via ATP synthase, which results in ATP production. Protons can also re-enter the mitochondrial matrix via pathways independent of ATP synthase, which ‘uncouples’ nutrient oxidation and proton efflux from ATP production.

The phrase “opthalmically acceptable” is employed herein to refer to those compounds, materials, pharmaceutical compositions, and/or dosage forms that are, within the scope of sound medical judgment, suitable for use in contact with the eyes of human beings and animals without excessive toxicity, irritation, allergic response, and/or other problem or complication, commensurate with a reasonable benefit/risk ratio.

The phrase “pharmaceutically acceptable” is employed herein to refer to those compounds, materials, pharmaceutical compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, PA, 1990, the disclosure of which is hereby incorporated by reference.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability, or be easier to formulate.

The terms “subject,” “individual” or “patient” are used interchangeably and as used herein are intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of enhancement of an immune response. The methods are particularly suitable for treating human patients having a disease or disorder described herein.

The terms “administration of” and or “administering a” compound should be understood to mean providing a compound described herein or a prodrug thereof to the individual in need of treatment.

The term “treating” or “treatment” as used herein refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

### EXAMPLES

Processes for preparing compounds of the present invention, such as Formulas A, I, Ia, II, IIa, III and IIIa, or for preparing intermediates useful for preparing compounds of Formulas A, I, Ia, II, IIa, III and IIIa or other formulas of the present disclosure are provided as further embodiments of the invention or are known in the art. While the following text may exemplify specific compounds and corresponding routes of synthesis, it is not intended to limit the scope of the invention to such particular reference or examples. Various modifications may be made by those skilled in the art, in view of practical and economic considerations, such as the source of the agents and specific conditions of reactions.

| Abbreviation | Chemical Name |
|---|---|
| (HCHO)ₙ | paraformaldehyde |
| ACN | acetonitrile |
| AcOH | acetic acid |
| AIBN | 2,2′-azo bisisobutyronitrile |
| AlMe₃ | trimethylaluminum |
| BBr₃ | boron tribromide |
| Boc₂O | di-tert-butyl dicarbonate |
| Cs₂CO₃ | cesium carbonate |
| DCM | dichloromethane |
| DIAD | diisopropyl azodicarboxylate |
| DIPEA | diisopropylethylamine |
| disodium; BLAH | diisobutylaluminum hydride |
| DMAP | N,N-4-dimethylaminopyridine |
| DMF | N,N-dimethylformamide |
| DMSO | dimethylsulfoxide |
| EDCI | 1-ethyl-3-(3-dimethylamino- |
| | propyl)carbodiimide hydrochloride |
| EtSH | ethanethiol |
| H₂O₂ | Hydrogen peroxide |
| HFIP | 1,1,1,3,3,3-hexafluoropropan-2-ol |
| HMTA | hexamethylenetetramine |
| HOBt | 1-hydroxybenzotriazole |
| ICF3 | trifluoro(iodo)methane |
| m-CPBA | m-chloroperoxybenzoic acid |
| MeI | iodomethane |
| MnO₂ | manganese dioxide |
| NaBH₃CN | sodium cyanoborohydride |
| NaBH₄ | sodium borohydride |
| NaCN | sodium cyanide |
| NaH | sodium hydride |
| NaIO₄ | sodium periodate |
| NaOMe | sodium methoxide |
| NaSEt | sodium thioethylate |
| NaSMe | sodium methyl mercaptide |
| NBS | N-bromosuccinimide |
| NCS | N-chlorosuccinimide |
| POCl₃ | phosphorus oxychloride |
| PPh₃ | triphenylphosphine |
| Py | pyridine |
| T₃P | 2,4,6-tripropyl-1,3,5,2,4,6- |
| | trioxatriphosphinane 2,4,6-trioxide |
| TBAF | tetra-n-butylammonium fluoride |
| TBDPSCl | tert-butylchlorodiphenylsilane |
| t-BuOK | Potassium tert-butoxide |
| TEA | triethylamine |
| TFA | trifluoroacetic acid |
| TMSI | iodotrimethylsilane |
| Zn(CN)₂ | zinc cyanide |

### Example 1

### 5-chloro-2-hydroxy-3-((methoxymethoxy)methyl)-N-(6-(trifluoromethyl)benzo[d]thiazol-2-yl)benzamide(1)

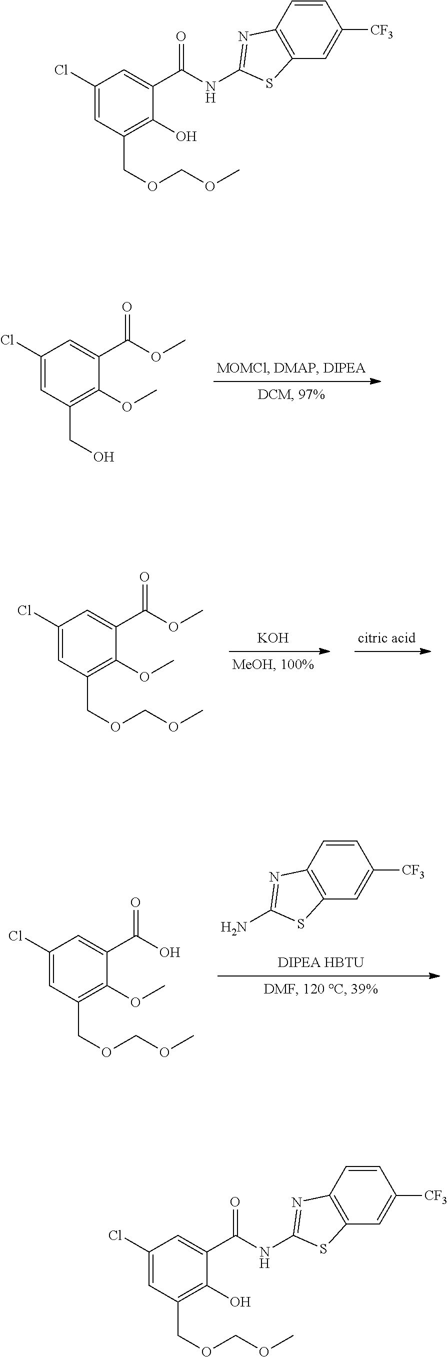

To a stirred solution of methyl 5-chloro-3-(hydroxymethyl)-2-methoxybenzoate (106 mg, 0.46 mmol) in DCM (5 mL) was added DIPEA (240 μL, 1.38 mmol), MOMCl (105 μL, 1.38 mmol) and followed by DMAP (3 mg, 0.023 mmol). The reaction mixture was stirred at rt overnight. After the reaction was completed, DCM and sat. Ammonia chloride solution were added. The organic layer was dried and concentrated, the residue was purified via silica gel column chromatography to give methyl 5-chloro-2-methoxy-3-((methoxymethoxy)methyl)benzoate (122 mg, 97%) as yellow oil. ¹H NMR (300 MHz, Chloroform-d) δ 7.72 (d, J=2.8 Hz, 1H), 7.58 (d, J=2.9 Hz, 1H), 4.73 (s, 2H), 4.64 (s, 2H), 3.90 (s, 3H), 3.83 (s, 3H), 3.40 (s, 3H).

To a stirred solution of methyl 5-chloro-2-methoxy-3-((methoxymethoxy)methyl)benzoate (122 mg, 0.445 mmol) in MeOH (5 mL) was added 2.2 mL 1N KOH solution. The resulting mixture was stirred at 60° C. overnight. After the mixture was cooled to rt, the reaction was partitioned between ethyl acetate and 2% citric acid. The ethyl acetate layer was washed with brine, dried over Na₂SO₄ and concentrated in vacuo. To this residue was added HBTU (98 mg, 0.258 mmol), DMF (3 mL) and DIPEA (187 μL, 1.075 mmol). The mixture was stirred for 10 minutes and then 6-(trifluoromethyl)benzo[d]thiazol-2-amine (47 mg, 0.215 mmol) was added. The resulting reaction was heated at 120° C. for 24 h. the mixture was cooled to rt, then separated between ethyl acetate and water. The organic layer was washed with brine, dried over Na₂SO₄ and concentrated in vacuo. Purification by column chromatography gave the 5-chloro-2-hydroxy-3-((methoxymethoxy)methyl)-N-(6-(trifluoromethyl)benzo[d]thiazol-2 yl)benzamide as a yellow solid (37 mg, 39%). ¹H NMR (400 MHz, Chloroform-d) δ 8.16 (s, 1H), 7.99 (d, J=2.5 Hz, 1H), 7.91 (d, J=6.1 Hz, 1H), 7.72 (d, J=6.1 Hz, 1H), 7.48 (d, J=2.5 Hz, 1H), 4.80 (s, 2H), 4.78 (s, 2H), 3.47 (s, 3H). MS (ESI) [M+Na]⁺ requires m/z 469.02, found m/z 468.55.

### Example 2

### 5-chloro-2-hydroxy-3-((2-methoxyethoxy)methyl)-N-(6-(trifluoromethyl)benzo[d]thiazol-2-yl)benzamide (2)

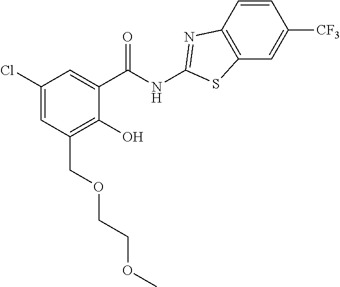 [SMILES: COCCOCc1cc(Cl)cc(C(=O)Nc2nc3ccc(C(F)(F)F)cc3s2)c1O]

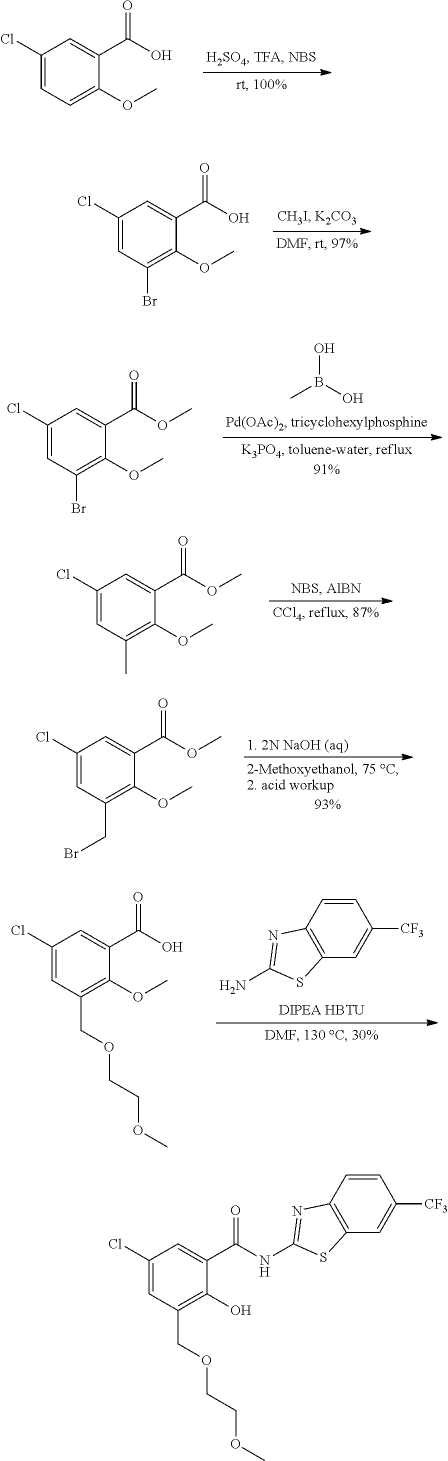

To a stirred solution of 5-chloro-2-methoxybenzoic acid (5.59 g, 30 mmol) in sulfuric acid (10.2 mL) and TFA (20.4 mL) at rt was added NBS (5.87 g, 33 mmol). The pale solution was stirred at rt overnight. The resulting pale suspension was carefully poured onto crushed ice. The mixture was extracted with ethyl acetate. The ethyl acetate layer was dried over Na₂SO₄ and concentrated under reduced pressure. The light yellow residue was suspended in a minimum amount of DCM. The solid was collected, washed with cold DCM and dried under vacuum to yield 3-bromo-5-chloro-2-methoxybenzoic acid as a white solid (8.00 g, 100%). ¹H NMR (300 MHz, acetone) δ 7.86 (d, 1H, J=3.0 Hz), 7.78(d, 1H, J=3.0 Hz), 3.91 (s, 3H).

To a stirred solution of 3-bromo-5-chloro-2-methoxybenzoic acid (6 g, 22.6 mmol) in DMF (30 mL) was added potassium carbonate (31 g, 226 mmol) followed by CH₃I (1.4 mL, 22.6 mmol). The mixture was stirred at rt for 24 h. Water was added and extracted with ethyl acetate two times. The combined organic layer was washed with water and brine and dried over sodium sulfate. The organic layer was filtered and the solvent removed in vacuo to yield a pale yellow oil (6.18 g, 97%).

A mixture of methyl 3-bromo-5-chloro-2-methoxybenzoate (756 mg, 2.7 mmol), methylboronic acid (324 mg, 5.4 mmol), Pd(OAc)₂ (24 mg, 0.11 mmol), tricyclohexylphosphine (68 mg, 0.24 mmol) and potassium phosphate tribasic (1.9 g, 8.96 mmol) was refluxed in toluene(10 mL) and water (1 mL) under N₂ overnight. After the reaction was cooled, saturated NH₄Cl solution was added and extracted with ethyl acetate two times. The combined ethyl acetate layer was dried over Na₂SO₄ and concentrated under reduced pressure. The residue was purified via silica gel column chromatography to give methyl 5-chloro-2-methoxy-3-methylbenzoate (520 mg, 91%) as a yellow oil. ¹H NMR (300 MHz, chloroform) δ 7.62 (d, 1H, J=3.0 Hz), 7.32(d, 1H, J=3.0 Hz), 3.92 (s, 3H), 3.82 (s, 3H), 2.30 (s, 3H).

To a flame dried flask was added NBS (183 mg, 1.03 mmol), AIBN (15 mg, 0.093 mmol) and a solution of methyl 5-chloro-2-methoxy-3-methylbenzoate (201 g, 0.93 mmol) in CCl₄ (10 mL). The suspension was refluxed in the dark overnight. The mixture was cooled to rt and concentrated. The residue was purified via silica gel column chromatography to give methyl 5-chloro-3-(bromomethyl)-2-methoxybenzoate as a colorless oil (236 mg, 87%). ¹H NMR (300 MHz, cdcl3) δ 7.76 (d, J=2.7 Hz, 1H), 7.54 (d, J=2.7 Hz, 1H), 4.51 (s, 2H), 3.96 (s, 3H), 3.94 (s, 3H).

To a stirred solution of methyl 5-chloro-3-(bromomethyl)-2-methoxybenzoate (236 mg, 0.805 mmol) in 2-methoxyethanol (10 mL) was added 2N NaOH solution (7 mL). The resulting mixture was stirred at 75° C. overnight and then concentrated in vacuo. The residue was dissolved in ethyl acetate and the resulting solution was washed with 2N HCl dried over sodium sulfate and concentrated in vacuo. The residure was triturated with ether to give 5-chloro-2-methoxy-3-((2-methoxyethoxy)methyl)benzoic acid (204 mg, 93%) as yellow oil. ¹H NMR (300 MHz, cdcl3) δ 10.02 (brs, 1H), 7.86 (d, J=2.8 Hz, 1H), 7.66 (d, J=1.4 Hz, 1H), 4.62 (s, 2H), 3.87 (s, 3H), 3.75-3.67 (m, 2H), 3.66-3.56 (m, 2H), 3.41 (s, 3H). MS (ESI) [M+Na]⁺ requires m/z 297.05, found m/z 296.6.

5-chloro-2-methoxy-3-((2-methoxyethoxy)methyl)benzoic acid (90 mg, 0.328 mmol) was dissolved in DMF (3 mL). HBTU (149 mg, 0.394 mmol) was added followed by DIPEA (286 μL, 1.64 mmol). The resulting mixture was stirred at rt for 15 mins, then 6-(trifluoromethyl)benzo[d]thiazol-2-amine (72 mg, 0.328 mmol) was added. The resulting mixture was stirred at 130° C. for 24 h. Saturated NH₄Cl solution was added and extracted with ethyl acetate two times. The combined ethyl acetate layer was dried over Na₂SO₄ and concentrated under reduced pressure. The residue was purified via silica gel column chromatography to give 5-chloro-2-hydroxy-3-((2-methoxyethoxy)methyl)-N-(6-(trifluoromethyl)benzo[d]thiazol-2-yl)benzamide as a yellow powder (34 mg, 30%). ¹H NMR (300 MHz, acetone) δ 8.37 (s, 1H), 8.02 (s, 1H), 7.81 (d, J=8.3 Hz, 1H), 7.69 (d, J=8.4 Hz, 1H), 7.41 (s, 1H), 4.91 (s, 2H), 3.87 (s, 3H), 3.53 (brs, 2H), 2.94 (brs, 2H). MS (ESI) [M+Na]⁺ requires m/z 483.04, found m/z 483.1.

### Example 3

### 5-chloro-2-hydroxy-3-(((2-methoxyethyl)(methyl)amino)methyl)-N-(6-(trifluoromethyl)benzo[d]thiazol-2-yl)benzamide (3) and 5-chloro-2-hydroxy-3-(((2-methoxyethyl)(methyl)amino)methyl)-N-(6-(trifluoromethyl)benzo[d]thiazol-2-yl)benzamide hydrochloride (3A)

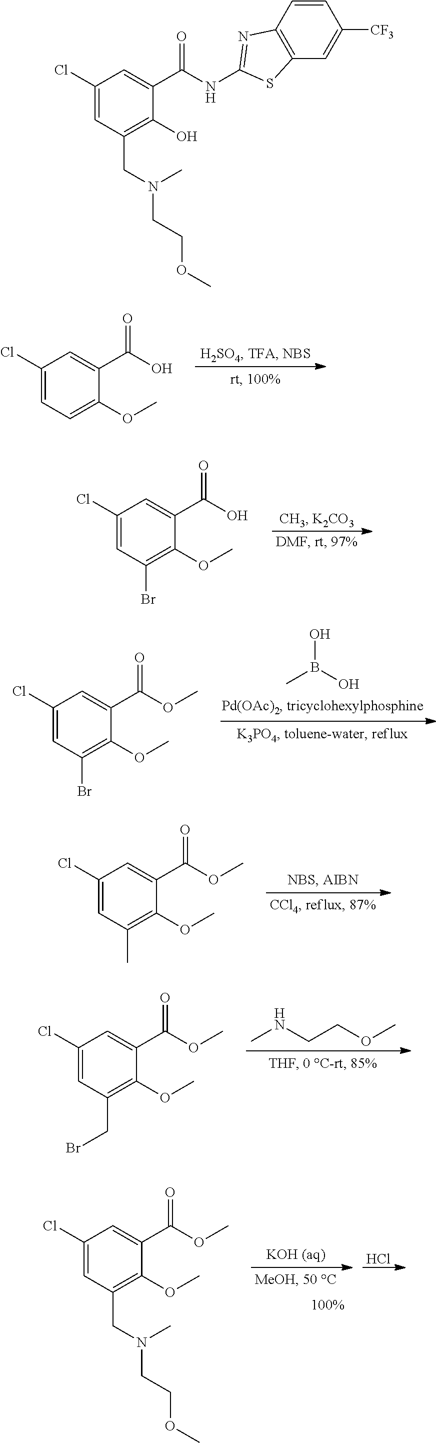

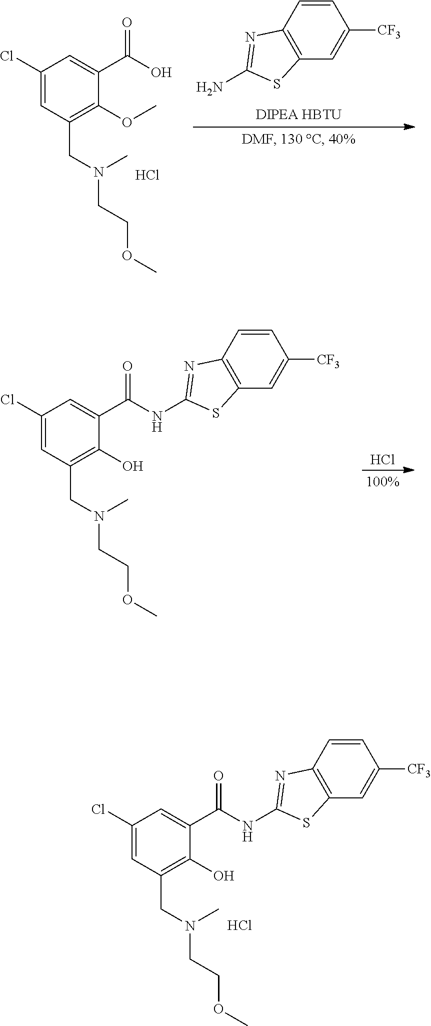

To a stirred solution of 5-chloro-2-methoxybenzoic acid (5.59 g, 30 mmol) in sulfuric acid (10.2 mL) and TFA (20.4 mL) at rt was added NBS (5.87 g, 33 mmol). The pale solution was stirred at rt overnight. The resulting pale suspension was carefully poured onto crushed ice. The mixture was extracted with ethyl acetate. The ethyl acetate layer dried over Na₂SO₄ and concentrated under reduced pressure. The light yellow residue was suspend in minimum amount of DCM. The solid was collected, washed with cold DCM and dried under vacuum to yield the 3-bromo-5-chloro-2-methoxybenzoic acid as a white solid (8.00 g, 100%). ¹H NMR (300 MHz, acetone) δ 7.86 (d, 1H, J=3.0 Hz), 7.78 (d, 1H, J=3.0 Hz), 3.91 (s, 3H).

To a stirred solution of 3-bromo-5-chloro-2-methoxybenzoic acid (6 g, 22.6 mmol) in DMF (30 mL) was added potassium carbonate (31 g, 226 mmol) followed by CH₃I (1.4 mL, 22.6 mmol). The mixture was stirred at rt for 24 h. Water was added and extracted with ethyl acetate two times. The combined organic layer was washed with water and brine and dried over sodium sulfate. The organic layer was filtered and the solvent removed in vacuo to yield a pale yellow oil (6.18 g, 97%).

A mixture of methyl 3-bromo-5-chloro-2-methoxybenzoate (756 mg, 2.7 mmol), methylboronic acid (324 mg, 5.4 mmol), Pd(OAc)₂ (24 mg, 0.11 mmol), tricyclohexylphosphine (68 mg, 0.24 mmol) and potassium phosphate tribasic (1.9 g, 8.96 mmol) was refluxed in toluene(10 mL) and water (1 mL) under N₂ overnight. After the reaction was cooled, saturated NH₄Cl solution was added and extracted with ethyl acetate two times. The combined ethyl acetate layer was dried over Na₂SO₄ and concentrated under reduced pressure. The residue was purified via silica gel column chromatography to give methyl 5-chloro-2-methoxy-3-methylbenzoate (520 mg, 91%) as a yellow oil. ¹H NMR (300 MHz, chloroform) δ 7.62 (d, 1H, J=3.0 Hz), 7.32 (d, 1H, J=3.0 Hz), 3.92 (s, 3H), 3.82 (s, 3H), 2.30 (s, 3H).

To a flame dried flask was added NBS (492 mg, 2.767 mmol), AIBN (57 mg, 0.346 mmol) and a solution of methyl 5-chloro-2-methoxy-3-methylbenzoate (495 mg, 2.306 mmol) in CCl4 (10 mL). The suspension was refluxed in the dark overnight. The mixture was cooled to rt and concentrated. The residue was purified via silica gel column chromatography to give methyl 5-chloro-3-(bromomethyl)-2-methoxybenzoate as a colorless oil (588 mg, 87%). ¹H NMR (300 MHz, cdcl3) δ 7.76 (d, J=2.7 Hz, 1H), 7.54 (d, J=2.7 Hz, 1H), 4.51 (s, 2H), 3.96 (s, 3H), 3.94 (s, 3H).

At 0° C., to a stirred solution of methyl 5-chloro-3-(bromomethyl)-2-methoxybenzoate (500 mg, 1.71 mmol) in THF (6 mL) was added 2-methoxy-N-methylethan-1-amine (370 μL, 3.41 mmol). The mixture was stirred at rt for 16 h. After completion of the reaction, the mixture was partitioned between NaHCO₃ and ethyl acetate. The aqueous layer was further extracted with ethyl acetate two times. The combined organic layer was washed with brine and dried over sodium sulfate. The solvent was removed under reduce pressure to afford methyl 5-chloro-2-methoxy-3-(((2-methoxyethyl)(methyl)amino)methyl)benzoate (437 mg, 85%) as a yellow oil. ¹H NMR (300 MHz, Chloroform-d) δ 7.67 (s, 2H), 3.91 (s, 3H), 3.81 (s, 2H), 3.61 (s, 2H), 3.52 (t, J=5.7 Hz, 2H), 3.34 (s, 3H), 2.64 (t, J=5.7 Hz, 2H), 2.29 (s, 3H). MS (ESI) [M+H]⁺ requires m/z 302.12, found m/z 301.60.

To a stirred solution of methyl 5-chloro-2-methoxy-3-(((2-methoxyethyl)(methyl)amino)methyl)benzoate (352 mg, 1.17 mmol) in MeOH (5 mL) was added 5.0 mL 1N KOH solution. The resulting mixture was stirred at 50° C. overnight. The solvent was evaporated out and 4N HCl in dioxane (2 mL) was added to the residue. The mixture was stirred for another 10 minutes before it was concentrated and dried under vacumn. To this residue was added HBTU (532 mg, 1.404 mmol), DMF (5 mL) and DIPEA (1.02 mL, 5.85 mmol). The mixture was stirred for 10 minutes and then 6-(trifluoromethyl)benzo[d]thiazol-2-amine (255 mg, 1.17 mmol) was added. The resulting reaction was heated at 130° C. for 24 hs. After cooling to rt, the mixture was separated between ethyl acetate and water. The organic layer was washed with brine, dried over Na₂SO₄ and concentrated in vacuo. Purification by column chromatography gave 5-chloro-2-hydroxy-3-(((2-methoxyethyl)(methyl)amino)methyl)-N-(6-(trifluoromethyl)benzo[d]thiazol-2-yl)benzamide as a yellow solid (220 mg, 40%). ¹H NMR (300 MHz, Methanol-d4) δ 8.24 (s, 1H), 7.93 (s, 1H), 7.84 (d, J=8.4 Hz, 1H), 7.69 (d, J=9.0 Hz, 1H), 7.31 (s, 1H), 4.31 (s, 2H), 3.78 (t, J=6.0 Hz, 2H), 3.44 (s, 3H), 3.35 (t, J=6.0 Hz, 2H), 2.85 (s, 3H). MS (ESI) [M+H]⁺ requires m/z 474.09, found m/z 473.55.

To a stirred solution of 5-chloro-2-hydroxy-3-(((2-methoxyethyl)(methyl)amino)methyl)-N-(6-(trifluoromethyl)benzo[d]thiazol-2-yl)benzamide (126 mg, 0.266 mmol) in THF (5 mL) was added 4.0N HCl in dioxane (70 μL, 0.266 mmol). The mixture was stirred at rt for 20 mins. The solvent was removed under reduce pressure and the resulting residue was washed with diethyl ether to afford 5-chloro-2-hydroxy-3-(((2-methoxyethyl)(methyl)amino)methyl)-N-(6-(trifluoromethyl)benzo[d]thiazol-2-yl)benzamide hydrochloride as a yellow solid (135 mg, 100%).

### Example 4

### 5-chloro-N-(2-chloro-4-(trifluoromethyl)phenyl)-2-hydroxy-3-((2-methoxyethoxy)methyl)benzamide (4)

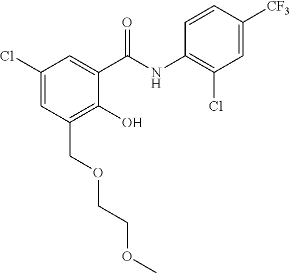 [SMILES: COCCOCc1cc(Cl)cc(C(=O)Nc2ccc(C(F)(F)F)cc2Cl)c1O]

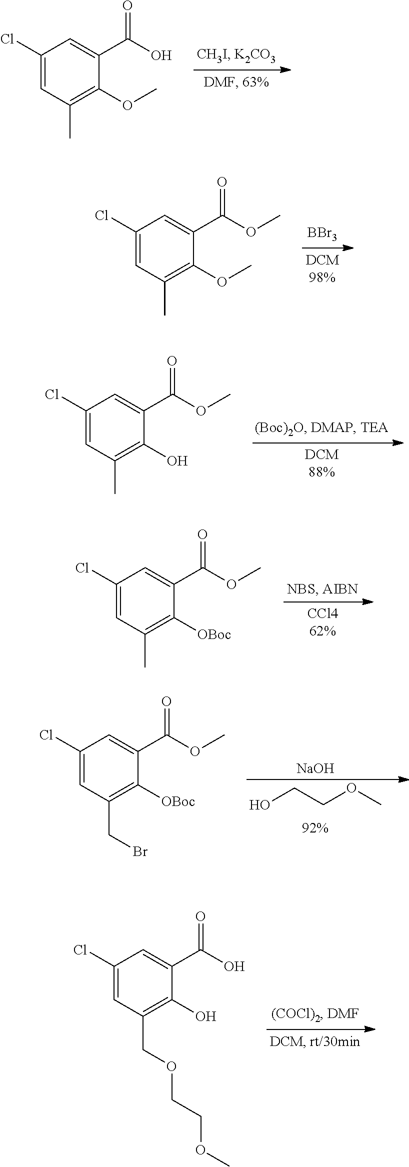

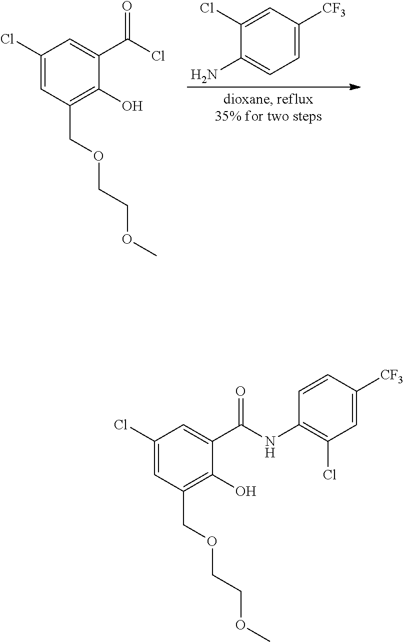 [SMILES: COCCOCc1cc(Cl)cc(C(=O)Cl)c1O ; Nc1ccc(C(F)(F)F)cc1Cl ; COCCOCc1cc(Cl)cc(C(=O)Nc2ccc(C(F)(F)F)cc2Cl)c1O]

To a stirred solution of 5-chloro-2-methoxy-3-methylbenzoic acid (1.80 g, 8.99 mmol) in DMF (10 mL) was added potassium carbonate (12.4 g, 89.9 mmol) followed by CH₃I (0.56 mL, 8.99 mmol). The mixture was stirred at rt for 24 h. Water was added and extracted with ethyl acetate two times. The combined organic layer was washed with water and brine and dried over sodium sulfate. The organic layer was filtered and the solvent removed in vacuo to yield methyl 5-chloro-2-methoxy-3-methylbenzoate as a pale yellow oil (1.13 g, 63%). ¹H NMR (300 MHz, cdcl₃) δ 7.52 (d, J=2.7 Hz, 1H), 7.22 (d, J=2.7 Hz, 1H), 3.83 (s, 3H), 3.74 (s, 3H), 2.21 (s, 3H).

At −78° C., to a stirred solution of methyl 5-chloro-2-methoxy-3-methylbenzoate (241 mg, 1.12 mmol) in anhydrous DCM (5 mL) was added BBr₃ (1.0. M in DCM, 2.25 mL) dropwise. After addition, the reaction was allowed to warm to rt slowly and the mixture was stirred at rt for 2 h. After completion of the reaction, the reaction mixture was cooled in an ice bath and MeOH and water were added to quench the reaction. The mixture was separated between DCM and water. The organic layer was washed with water and brine, dried over sodium sulfate and concentrated in vacuo to afford methyl 5-chloro-2-hydroxy-3-methylbenzoate as pale yellow solid (220 mg, 98%), which was used in the next step without further purification.

To a stirred solution of methyl 5-chloro-2-hydroxy-3-methylbenzoate (220 mg, 1.1 mmol) in anhydrous DCM (5 mL) was added pyridine (443 μL, 5.5 mmol), (Boc)₂O (504 mg, 2.30 mmol) and DMAP (13 mg, 0.11 mmol). The resulting mixture was stirred at rt for 2 days. After completion of the reaction, solvent was evaporated and the resulting residue was purified via silica gel column chromatography to yield methyl 2-((tert-butoxycarbonyl)oxy)-5-chloro-3-methylbenzoate (290 mg, 88%) as a colorless oil. ¹H NMR (300 MHz, cdcl₃) δ 7.80 (dd, J=2.7, 0.6 Hz, 1H), 7.40 (dd, J=2.7, 0.7 Hz, 1H), 3.89 (s, 3H), 2.26 (s, 3H), 1.58 (s, 9H).

To a flame dried flask was added NBS (205 mg, 1.156 mmol), AIBN (24 mg, 0.145 mmol) and a solution of methyl 2-((tert-butoxycarbonyl)oxy)-5-chloro-3-methylbenzoate (289 mg, 0.963 mmol) in CCl₄ (5 mL). The suspension was refluxed in the dark overnight. The mixture was cooled to rt and concentrated. The residue was purified via silica gel column chromatography to give methyl 3-(bromomethyl)-2-((tert-butoxycarbonyl)oxy)-5-chlorobenzoate as a colorless oil (220 mg, 62%). ¹H NMR (300 MHz, cdcl₃) δ 7.95 (d, J=2.2 Hz, 1H), 7.61 (d, J=2.3 Hz, 1H), 4.45 (s, 2H), 3.91 (s, 3H), 1.58 (s, 9H).

To a stirred solution of methyl 3-(bromomethyl)-2-((tert-butoxycarbonyl)oxy)-5-chlorobenzoate (220 mg, 0.582 mmol) in 2-methoxyethanol (10 mL) was added 2N NaOH solution (5 mL). The resulting mixture was stirred at rt overnight and then concentrated in vacuo. The residue was dissolved in ethyl acetate and the resulting solution was washed with 2N HCl dried over sodium sulfate and concentrated in vacuo. The residue was triturated with ether to give 5-chloro-2-hydroxy-3-((2-methoxyethoxy)methyl)benzoic acid (145 mg, 92%) as a white oil. ¹H NMR (300 MHz, cdcl₃) δ 10.76 (s, 2H), 7.67 (d, J=2.6 Hz, 1H), 7.59 (d, J=2.5 Hz, 1H), 4.61 (s, 2H), 3.79 (dd, J=5.9, 2.7 Hz, 2H), 3.72 (dd, J=5.9, 2.8 Hz, 2H), 3.49 (s, 3H). MS (ESI) [M+Na]⁺ requires m/z 283.03, found m/z 282.55.

5-chloro-2-hydroxy-3-((2-methoxyethoxy)methyl)benzoic acid (65 mg, 0.249 mmol) was dissolved in THF (3.0 mL), followed by the addition of a catalytic amount of DMF (1 drop) and oxalyl chloride (26 μL, 0.299 mmol) respectively. The reaction was allowed to stir at rt for 30 min and was concentrated in vacuo. The residue was re-dissolved in dioxane(5.0 mL) and 2-chloro-4-(trifluoromethyl)aniline (35 μL, 0.25 mmol) was added. The mixture was refluxed overnight. Solvent was evaporated and the resulting residue was purified via silica gel column chromatography to yield -chloro-N-(2-chloro-4-(trifluoromethyl)phenyl)-2-hydroxy-3-((2-methoxyethoxy)methyl)benzamide (35 mg, 35%) as a white solid. ¹H NMR (500 MHz, cdcl₃) δ 10.25 (s, 1H), 10.20 (s, 1H), 8.76 (d, J=8.6 Hz, 1H), 7.96 (d, J=2.6 Hz, 1H), 7.69 (d, J=1.7 Hz, 1H), 7.58 (dd, J=8.8, 2.1 Hz, 1H), 7.36 (d, J=2.6 Hz, 1H), 4.73 (s, 2H), 3.82-3.76 (m, 2H), 3.66-3.62 (m, 2H), 3.45 (s, 3H). MS (ESI) [M+Na]⁺ requires m/z 460.03, found m/z 459.95.

### Example 5

### 5-chloro-2-hydroxy-3-((2-methoxyethoxy)methyl)-N-(6-(trifluoromethoxy)benzo[d]thiazol-2-yl)benzamide (5)

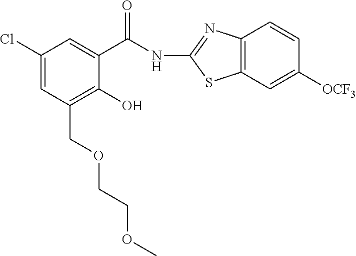 [SMILES: COCCOCc1cc(Cl)cc(C(=O)Nc2nc3ccc(OC(F)(F)F)cc3s2)c1O]

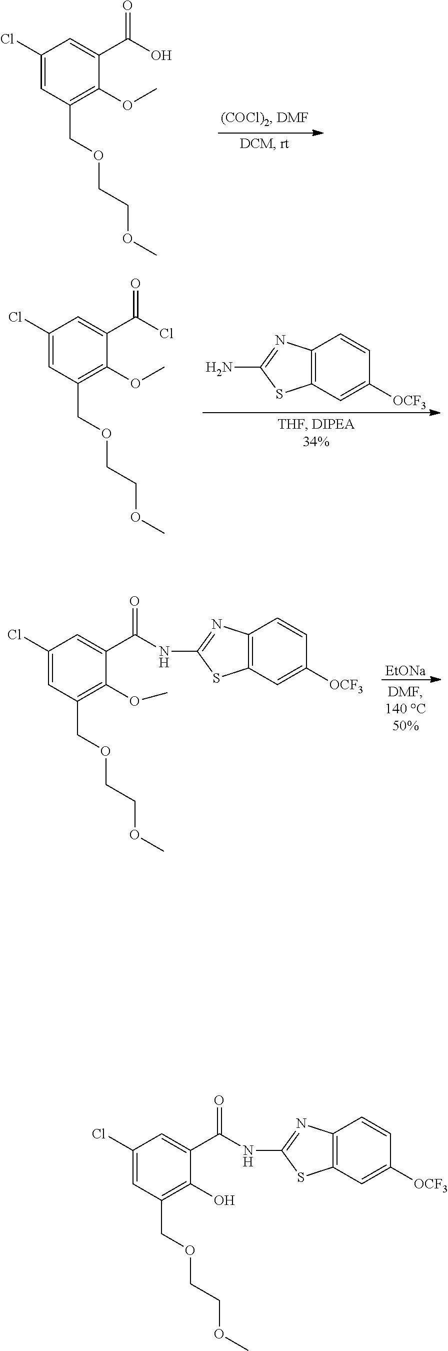

5-chloro-2-hydroxy-3-((2-methoxyethoxy)methyl)benzoic acid (77 mg, 0.28 mmol, Example 2) was dissolved in DCM (3.0 mL), followed by the addition of a catalytic amount of DMF (1 drop) and oxalyl chloride (40 μL, 0.34 mmol) respectively. The reaction was allowed to stir at rt for 30 minutes and concentrated in vacuo. The residue was re-dissolved in THF (5.0 mL), and Hunig's base (59 μL, 0.34 mmol) and 6-(trifluoromethoxy)benzo[d]thiazol-2-amine (65 mg, 0.28 mmol) were added. The mixture was stirred at rt for 48 hours before silica gel was added to quench the reaction. Solvent was evaporated and the resulting residue was purified via silica gel column chromatography to yield 5-chloro-2-methoxy-3-((2-methoxyethoxy)methyl)-N-(6-(trifluoromethoxy)benzo[d]thiazol-2-yl)benzamide (27 mg, 34% yield) as a white solid. ¹H NMR (500 MHz, cdcl₃) δ 8.13 (d, J=2.8 Hz, 1H), 7.83 (d, J=8.8 Hz, 1H), 7.73 (d, J=2.8 Hz, 2H), 7.34 (ddd, J=8.8, 2.4, 0.8 Hz, 1H), 4.66 (s, 2H), 3.99 (s, 3H), 3.75-3.72 (m, 2H), 3.62 (dd, J=3.9, 2.5 Hz, 2H), 3.41 (s, 3H). MS (ESI) [M+H]⁺ requires m/z 491.07, found m/z 491.10.

A solution of 5-chloro-2-methoxy-3-((2-methoxyethoxy)methyl)-N-(6-(trifluoromethoxy)benzo[d]thiazol-2-yl)benzamide (27 mg, 0.055 mmol) in DMF (3 mL) is mixed with sodium ethoxide (18 mg, 0.275 mmol) and the resulting suspension is heated at 140° C. overnight. After completion of the reaction, 1N HCl was added to the reaction and extracted with ethyl acetate two times. The combined ethyl acetate layer was dried over sodium sulfate and concentrated under reduced pressure. The residue was purified via silica gel column chromatography to give 5-chloro-2-hydroxy-3-((2-methoxyethoxy)methyl)-N-(6-(trifluoromethoxy)benzo[d]thiazol-2-yl)benzamide (13 mg, 50%) as a yellow solid. ¹H NMR (500 MHz, cdcl₃) δ 8.13 (d, J=2.8 Hz, 1H), 7.83 (d, J=8.8 Hz, 1H), 7.73 (d, J=2.8 Hz, 2H), 7.34 (ddd, J=8.8, 2.4, 0.8 Hz, 1H), 4.66 (s, 2H), 3.75-3.72 (m, 2H), 3.62 (dd, J=3.9, 2.5 Hz, 2H), 3.41 (s, 3H). MS (ESI) [M+H]⁺ requires m/z 477.05, found m/z 477.30.

### Example 6

### 5-chloro-2-hydroxy-3-((2-methoxyethoxy)methyl)-N-(5-(trifluoromethyl)pyrazin-2-yl)benzamide (6)

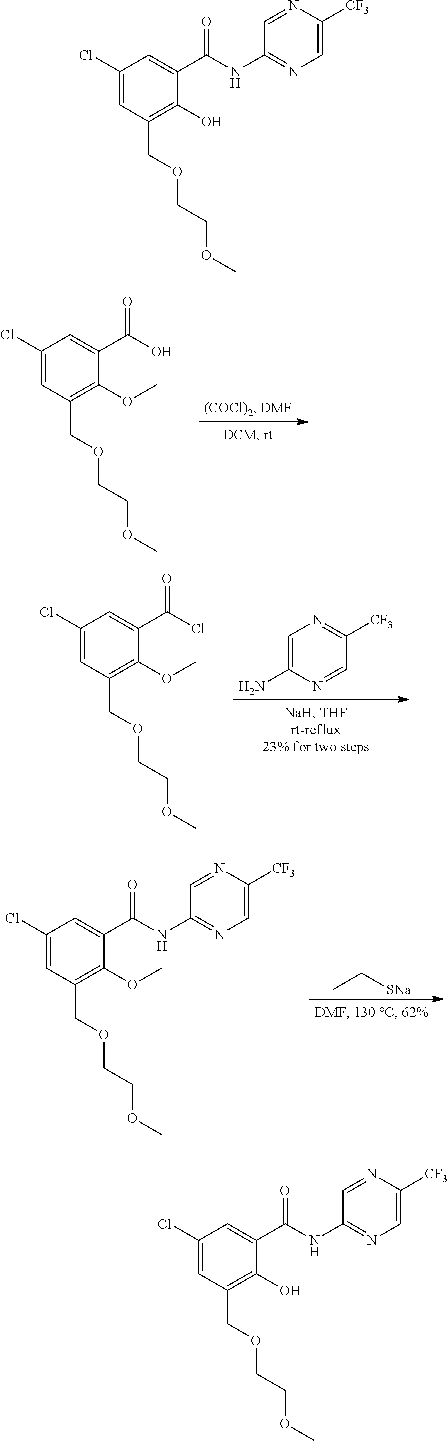

5-chloro-2-methoxy-3-((2-methoxyethoxy)methyl)benzoic acid (90 mg, 0.33 mmol, Example 2) was dissolved in DCM (3.0 mL), followed by the addition of catalytic amount of DMF (10 μL) and oxalyl chloride (34 μL, 0.39 mmol) respectively. The reaction was allowed to stir at rt for 30 minutes, concentrated in vacuo, and the 5-chloro-2-methoxy-3-((2-methoxyethoxy)methyl)benzoyl chloride residue was re-dissolved in THF (3.0 mL). In another flask, 5-(trifluoromethyl)pyrazin-2-amine (54.0 mg, 0.33 mmol) was dissolved in THF (3.0 mL) followed by addition of NaH (16.0 mg, 0.439 mmol, 60% in mineral oil). The mixture was stirred for 10 minutes before it was added dropwise at rt to the flask containing the freshly prepared 5-chloro-2-methoxy-3-((2-methoxyethoxy)methyl)benzoyl chloride. The reaction was stirred at rt for 2 h before silica gel was added to quench the reaction. Solvent was evaporated and the resulting residue was purified via silica gel column chromatography to yield 5-chloro-2-methoxy-3-((2-methoxyethoxy)methyl)-N-(5-(trifluoromethyl)pyrazin-2-yl)benzamide as a white solid (32.0 mg, 23% yield). ¹H NMR (300 MHz, cdcl₃) δ 10.54 (s, 1H), 9.82 (d, J=1.1 Hz, 1H), 8.67 (s, 1H), 8.11 (d, J=2.8 Hz, 1H), 7.71 (d, J=2.8 Hz, 1H), 4.68 (s, 2H), 3.95 (s, 3H), 3.79-3.71 (m, 2H), 3.67-3.59 (m, 2H), 3.43 (s, 3H). MS (ESI) [M+H]⁺ requires m/z 420.10, found m/z 420.30.

A solution of 5-chloro-2-methoxy-3-((2-methoxyethoxy)methyl)-N-(5-(trifluoromethyl)pyrazin-2-yl)benzamide (32 mg, 0.076 mmol) in DMF (3 mL) is mixed with sodium ethanethiolate (32 mg, 0.382 mmol) and the resulting suspension is heated at 130° C. overnight. After completion of the reaction, 1N HCl was added to the reaction and extracted with ethyl acetate two times. The combined ethyl acetate layer was dried over sodium sulfate and concentrated under reduced pressure. The residue was purified via silica gel column chromatography to give 5-chloro-2-hydroxy-3-((2-methoxyethoxy)methyl)-N-(5-(trifluoromethyl)pyrazin-2-yl)benzamide (18 mg, 62%) as a yellow solid. ¹H NMR (300 MHz, acetone) δ 9.66 (s, 1H), 8.82 (s, 1H), 8.13 (s, 1H), 7.56 (s, 1H), 4.74 (s, 2H), 3.88-3.74 (m, 2H), 3.74-3.61 (m, 2H), 3.41 (s, 3H). MS (ESI) [M+H]⁺ requires m/z 406.08, found m/z 406.20.

### Example 7

### 5-chloro-2-hydroxy-3-((2-hydroxyethoxy)methyl)-N-(6-(trifluoromethyl)benzo[d]thiazol-2-yl)benzamide (7)

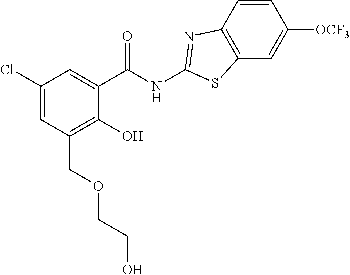 [SMILES: O=C(Nc1nc2ccc(OC(F)(F)F)cc2s1)c1cc(Cl)cc(COCCO)c1O]

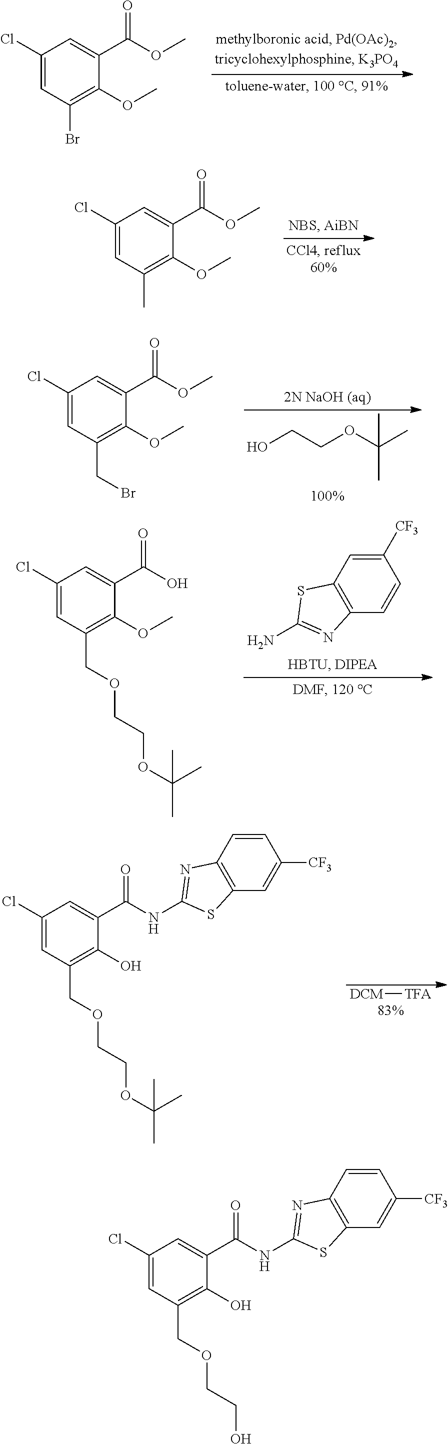

A mixture of methyl 3-bromo-5-chloro-2-methoxybenzoate (756 mg, 2.7 mmol, Example 2), methylboronic acid (324 mg, 5.4 mmol), Pd(OAc)₂ (24 mg, 0.11 mmol), tricyclohexylphosphine (68 mg, 0.24 mmol) and potassium phosphate tribasic (1.9 g, 8.96 mmol) was refluxed in toluene(10 mL) and water (1 mL) under N₂ overnight. After the reaction was cooled, saturated NH₄Cl solution was added and extracted with ethyl acetate two times. The combined ethyl acetate layer was dried over sodium sulfate and concentrated under reduced pressure. The residue was purified via silica gel column chromatography to give methyl 5-chloro-2-methoxy-3-methylbenzoate (520 mg, 91%) as a yellow oil. ¹H NMR (300 MHz, chloroform) δ 7.62 (d, 1H, J=3.0 Hz), 7.32 (d, 1H, J=3.0 Hz), 3.92 (s, 3H), 3.82 (s, 3H), 2.30 (s, 3H).

To a flame dried flask was added NBS (183 mg, 1.03 mmol), AIBN (15 mg, 0.093 mmol) and a solution of methyl 5-chloro-2-methoxy-3-methylbenzoate (201 mg, 0.93 mmol) in CCl₄ (10 mL). The suspension was refluxed in the dark overnight. The mixture was cooled to rt and concentrated. The residue was purified via silica gel column chromatography to give methyl 5-chloro-3-(bromomethyl)-2-methoxybenzoate as a colorless oil (236 mg, 87%). ¹H NMR (300 MHz, cdcl₃) δ 7.76 (d, J=2.7 Hz, 1H), 7.54 (d, J=2.7 Hz, 1H), 4.51 (s, 2H), 3.96 (s, 3H), 3.94 (s, 3H).

To a stirred solution of methyl 5-chloro-3-(bromomethyl)-2-methoxybenzoate (222 mg, 0.76 mmol) in 2-(tert-butoxy)ethan-1-ol (4 mL) was added 2N NaOH solution (2 mL). The resulting mixture was stirred at rt for 4 h and then concentrated in vacuo. The residue was dissolved in ethyl acetate and the resulting solution was washed with 2N HCl dried over sodium sulfate and concentrated in vacuo. The residure was triturated with ether to give 3-((2-(tert-butoxy)ethoxy)methyl)-5-chloro-2-methoxybenzoic acid as a yellow oil (100%). MS (ESI) [M−H]⁻ requires m/z 315.10, found m/z 315.60.

To a stirred solution of 3-((2-(tert-butoxy)ethoxy)methyl)-5-chloro-2-methoxybenzoic acid (240 mg, 0.76 mmol) in DMF (5 mL) was added HBTU (345 mg, 0.91 mmol) and DIPEA (662 μL, 3.8 mmol). The mixture was stirred for 10 minutes and then 6-(trifluoromethyl)benzo[d]thiazol-2-amine (165 mg, 0.76 mmol) was added. The resulting reaction was heated at 120° C. for 24 h. After cooled to rt, the mixture was separated between ethyl acetate and water. The organic layer was washed with brine, dried over sodium sulfate and concentrated in vacuo. Purification by column chromatography gave 3-((2-(tert-butoxy)ethoxy)methyl)-5-chloro-2-hydroxy-N-(6-(trifluoromethyl)benzo[d]thiazol-2-yl)benzamide as a yellow solid (27 mg, 10%). ¹H NMR (300 MHz, acetone) δ 8.35 (s, 1H), 7.96 (s, 1H), 7.72 (dd, J=19.4, 8.5 Hz, 2H), 7.41 (s, 1H), 4.89 (s, 2H), 4.06 (s, 2H), 3.82 (s, 2H), 1.15 (s, 9H). MS (ESI) [M+H]⁺ requires m/z 503.10, found m/z 503.20.

To a stirred solution of 3-((2-(tert-butoxy)ethoxy)methyl)-5-chloro-2-hydroxy-N-(6-(trifluoromethyl)benzo[d]thiazol-2-yl)benzamide (27 mg, 0.054 mmol) in DCM (3 mL) was added TFA (1 mL) and the mixture was stirred at rt overnight. The solvent was evaporated out and the remaining residue was subjected to flash column to afford 5-chloro-2-hydroxy-3-((2-hydroxyethoxy)methyl)-N-(6-(trifluoromethyl)benzo[d]thiazol-2-yl)benzamide as a yellow solid (19 mg, 83%). ¹H NMR (300 MHz, acetone) δ 8.35 (s, 1H), 7.96 (s, 1H), 7.72 (dd, J=19.4, 8.5 Hz, 2H), 7.41 (s, 1H), 4.89 (s, 2H), 4.06 (s, 2H), 3.82 (s, 2H). MS (ESI) [M+H]⁺ requires m/z 447.04, found m/z 447.10.

### Example 8

### 5-chloro-N-(6-fluorobenzo[d]thiazol-2-yl)-2-hydroxy-3-((2-methoxyethoxy)methyl)benzamide (8)

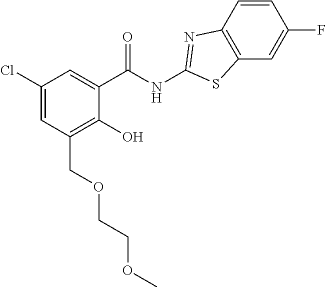 [SMILES: COCCOCc1cc(Cl)cc(C(=O)Nc2nc3ccc(F)cc3s2)c1O]

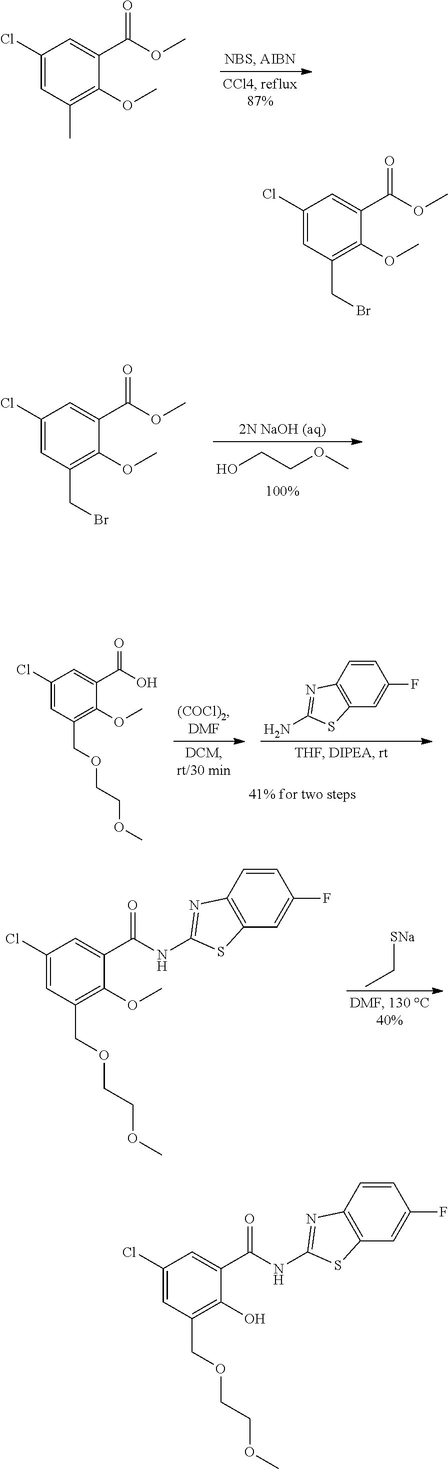

To a flame dried flask was added NBS (183 mg, 1.03 mmol), AIBN (15 mg, 0.093 mmol) and a solution of methyl 5-chloro-2-methoxy-3-methylbenzoate (201 g, 0.93 mmol) in CCl4 (10 mL). The suspension was refluxed in the dark overnight. The mixture was cooled to rt and concentrated. The residue was purified via silica gel column chromatography to give methyl 3-(bromomethyl)-5-chloro-2-methoxybenzoate as a colorless oil (236 mg, 87%). ¹H NMR (300 MHz, cdcl₃) δ 7.76 (d, J=2.7 Hz, 1H), 7.54 (d, J=2.7 Hz, 1H), 4.51 (s, 2H), 3.96 (s, 3H), 3.94 (s, 3H).

To a stirred solution of 3-(bromomethyl)-5-chloro-2-methoxybenzoate (180 mg, 0.614 mmol) in 2-methoxyethanol (5 mL) was added 2N NaOH solution (4 mL). The resulting mixture was stirred at rt overnight and then concentrated in vacuo. The residue was dissolved in ethyl acetate and the resulting solution was washed with 2N HCl dried over sodium sulfate and concentrated in vacuo. The residure was triturated with ether to give 5-chloro-2-methoxy-3-((2-methoxyethoxy)methyl)benzoic acid (170 mg, 100%) as yellow oil. ¹H NMR (300 MHz, cdcl₃) δ 10.02 (brs, 1H), 7.86 (d, J=2.8 Hz, 1H), 7.66 (d, J=1.4 Hz, 1H), 4.62 (s, 2H), 3.87 (s, 3H), 3.75-3.67 (m, 2H), 3.66-3.56 (m, 2H), 3.41 (s, 3H). MS (ESI) [M+Na]⁺ requires m/z 297.05, found m/z 296.6.

5-chloro-2-methoxy-3-((2-methoxyethoxy)methyl)benzoic acid (85 mg, 0.31 mmol) was dissolved in DCM (3.0 mL), followed by the addition of a catalytic amount of DMF (1 drop) and oxalyl chloride (32 μL, 0.372 mmol) respectively. The reaction was allowed to stir at rt for 30 minutes and was concentrated in vacuo. The residue was re-dissolved in THF (5.0 mL), and Hunig's base (162 μL, 0.93 mmol) and 6-fluorobenzo[d]thiazol-2-amine (52 mg, 0.31 mmol) were added. The mixture was stirred at rt for 48 hours before silica gel was added to quench the reaction. Solvent was evaporated and the resulting residue was purified via silica gel column chromatography to yield 5-chloro-N-(6-fluorobenzo[d]thiazol-2-yl)-2-methoxy-3-((2-methoxyethoxy)methyl)benzamide (52 mg, 41%) as a white solid. MS (ESI) [M+H]⁺ requires m/z 425.08, found m/z 425.10.

A solution of 5-chloro-N-(6-fluorobenzo[d]thiazol-2-yl)-2-methoxy-3-((2-methoxyethoxy)methyl)benzamide (52 mg, 0.123 mmol) in DMF (3 mL) was mixed with sodium ethanethiolate (52 mg, 0.613 mmol) and the resulting suspension was heated at 130° C. overnight. After completion of the reaction, 1N HCl was added to the reaction and extracted with ethyl acetate two times. The combined ethyl acetate layer was dried over sodium sulfate and concentrated under reduced pressure. The residue was purified via silica gel column chromatography to give 5-chloro-N-(6-fluorobenzo[d]thiazol-2-yl)-2-hydroxy-3-((2-methoxyethoxy)methyl)benzamide (20 mg, 40%) as a yellow solid. ¹H NMR (300 MHz, acetone) δ 8.05 (s, 1H), 7.77 (dd, J=8.4, 2.5 Hz, 1H), 7.61 (s, 1H), 7.47 (s, 1H), 7.20 (t, J=8.8 Hz, 1H), 4.79 (s, 2H), 3.83-3.72 (m, 4H), 3.47 (s, 3H). MS (ESI) [M+H]⁺ requires m/z 411.06, found m/z 411.10.

### Example 9

### 5-chloro-2-hydroxy-3-(methoxymethyl)-N-(6-(trifluoromethyl)benzo[d]thiazol-2-yl)benzamide (9)

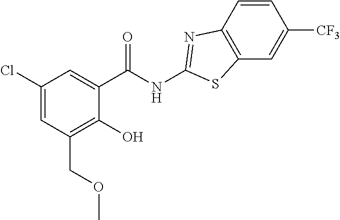 [SMILES: COCc1cc(Cl)cc(C(=O)Nc2nc3ccc(C(F)(F)F)cc3s2)c1O]

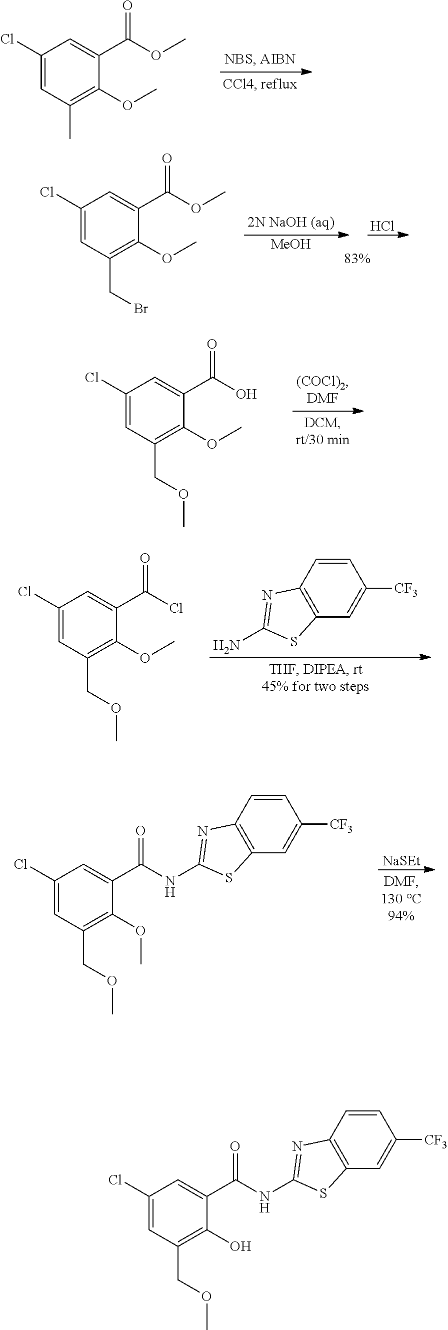

To a flame dried flask was added NBS (183 mg, 1.03 mmol), AIBN (15 mg, 0.093 mmol) and a solution of methyl 5-chloro-2-methoxy-3-methylbenzoate (201 g, 0.93 mmol) in CCl4 (10 mL). The suspension was refluxed in the dark overnight. The mixture was cooled to rt and concentrated. The residue was purified via silica gel column chromatography to give methyl 3-(bromomethyl)-5-chloro-2-methoxybenzoate as a colorless oil (236 mg, 87%). ¹H NMR (300 MHz, cdcl₃) δ 7.76 (d, J=2.7 Hz, 1H), 7.54 (d, J=2.7 Hz, 1H), 4.51 (s, 2H), 3.96 (s, 3H), 3.94 (s, 3H).

To a stirred solution of methyl 3-(bromomethyl)-5-chloro-2-methoxybenzoate (270 mg, 0.921 mmol) in methanol (10 mL) was added 2N NaOH solution (7 mL). The resulting mixture was stirred at rt overnight and then concentrated in vacuo. The residue was dissolved in ethyl acetate and the resulting solution was washed with 2N HCl dried over sodium sulfate and concentrated in vacuo. The residure was triturated with ether to give 5-chloro-2-methoxy-3-(methoxymethyl)benzoic acid (217 mg, 83%) as yellow oil which was used in the next step without further purification. MS (ESI) [M−H]⁻ requires m/z 229.02, found m/z 229.40.

5-chloro-2-methoxy-3-(methoxymethyl)benzoic acid (217 mg, 0.94 mmol) was dissolved in DCM (5.0 mL), followed by the addition of a catalytic amount of DMF (1 drop) and oxalyl chloride (97 μL, 1.13 mmol) respectively. The reaction was allowed to stir at rt for 30 min and concentrated in vacuo. The residue was re-dissolved in THE (5.0 mL), and Hunig's base (197 μL, 1.13 mmol) and 6-(trifluoromethyl)benzo[d]thiazol-2-amine (206 mg, 0.94 mmol) were added. The mixture was stirred at rt for 48 hours before silica gel was added to quench the reaction. Solvent was evaporated and the resulting residue was purified via silica gel column chromatography to yield 5-chloro-2-methoxy-3-(methoxymethyl)-N-(6-(trifluoromethyl)benzo[d]thiazol-2-yl)benzamide (165 mg, 45%) as a yellow solid. MS (ESI) [M+H]⁺ requires m/z 431.05, found m/z 431.10.

A solution of 5-chloro-2-methoxy-3-(methoxymethyl)-N-(6-(trifluoromethyl)benzo[d]thiazol-2-yl)benzamide (100 mg, 0.23 mmol) in DMF (5 mL) was mixed with sodium ethanethiolate (97 mg, 1.16 mmol) and the resulting suspension was heated at 130° C. overnight. After completion of the reaction, 1N HCl was added to the reaction and extracted with ethyl acetate two times. The combined ethyl acetate layer was dried over sodium sulfate and concentrated under reduced pressure. The residue was purified via silica gel column chromatography to give 5-chloro-2-hydroxy-3-(methoxymethyl)-N-(6-(trifluoromethyl)benzo[d]thiazol-2-yl)benzamide (90 mg, 94%) as a white solid. ¹H NMR (300 MHz, cdcl₃) δ 8.16 (s, 1H), 8.13 (d, J=2.6 Hz, 1H), 7.87 (d, J=8.6 Hz, 1H), 7.73 (d, J=8.9 Hz, 1H), 7.49 (d, J=2.6 Hz, 1H), 4.67 (s, 2H), 3.55 (s, 3H). MS (ESI) [M−H]⁻ requires m/z 415.02, found m/z 415.40.

### Example 10

### 5-chloro-2-hydroxy-3-(pyridin-3-yl)-N-(6-(trifluoromethyl)benzo[d]thiazol-2-yl)benzamide(10) and 5-chloro-2-hydroxy-3-(pyridin-3-yl)-N-(6-(trifluoromethyl)benzo[d]thiazol-2-yl)benzamide hydrochloride (10A)

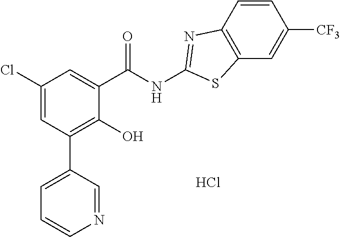 [SMILES: O=C(Nc1nc2ccc(C(F)(F)F)cc2s1)c1cc(Cl)cc(-c2cccnc2)c1O ; Cl]

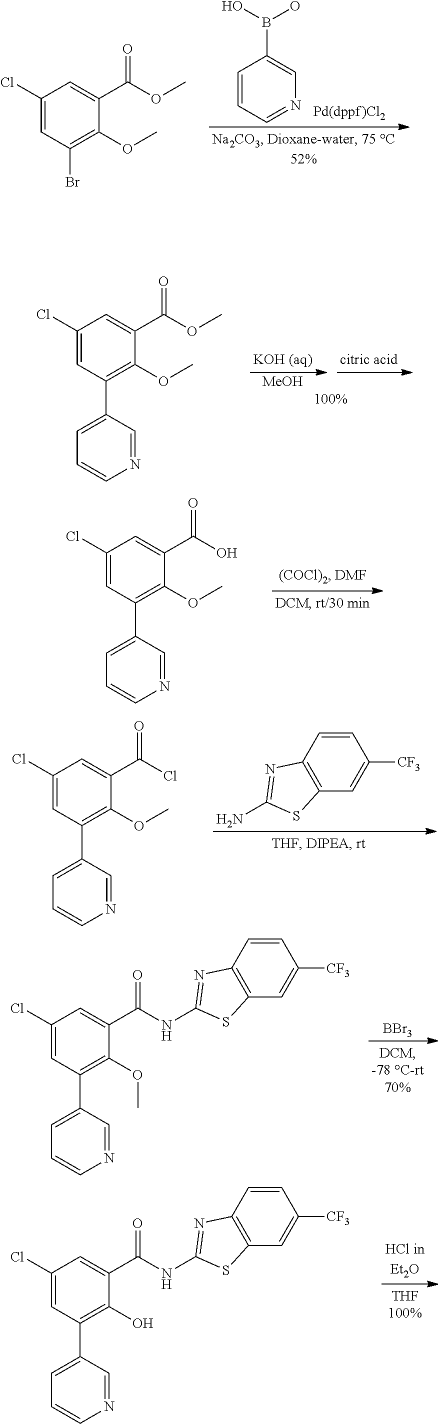

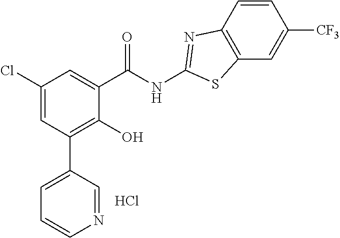 [SMILES: O=C(Nc1nc2ccc(C(F)(F)F)cc2s1)c1cc(Cl)cc(-c2cccnc2)c1O ; Cl]

A mixture of methyl 3-bromo-5-chloro-2-methoxybenzoate (280 mg, 1.0 mmol, Example 2), 3-pyridine boronic acid (184 mg, 1.5 mmol), PdCl₂(dpppf)-DCM(81 mg, 0.1 mmol) and 2M sodium carbonate (2 mL) in dioxane (5 mL) was heated to 75° C. overnight under nitrogen. After cooling, the reaction mixture was partitioned between water and ethyl acetate. The organic layer was washed with brine and dried over sodium sulfate. After concentration, the residue was purified via silica gel column chromatography to give methyl 5-chloro-2-methoxy-3-(pyridin-3-yl)benzoate (140 mg, 52%) as a white solid. ¹H NMR (300 MHz, cdcl₃) δ 8.74 (d, J=40.1 Hz, 2H), 7.95 (d, J=6.3 Hz, 1H), 7.81 (d, J=1.5 Hz, 1H), 7.47 (brs, J=17.7 Hz, 2H), 3.96 (s, 3H), 3.52 (s, 3H). MS (ESI) [M+H]⁺ requires m/z 278.06, found m/z 278.20.

To a stirred solution of methyl 5-chloro-2-methoxy-3-(pyridin-3-yl)benzoate (140 mg, 0.505 mmol) in MeOH (4 mL) was added 2.5 mL 1N KOH solution. The resulting mixture was stirred at rt overnight. The solvent was evaporated out and 10% citric acid was added to the residue to pH=3. The mixture was extracted with DCM two times. The combined organic layer was dried over sodium sulfate and concentrated to afford 5-chloro-2-methoxy-3-(pyridin-3-yl)benzoic acid (132 mg, 100%) which was used directly in the next step without further purification as a yellow solid. MS (ESI) [M+H]⁺ requires m/z 264.04, found m/z 264.20.

5-chloro-2-methoxy-3-(pyridin-3-yl)benzoic acid (132 mg, 0.50 mmol) was dissolved in DCM (3.0 mL) and THF (3.0 mL), followed by the addition of a catalytic amount of DMF (1 drop) and oxalyl chloride (52 μL, 0.60 mmol) respectively. The reaction was allowed to stir at rt for 30 minutes and then concentrated in vacuo. The residue was re-dissolved in THF (5.0 mL), and Hunig's base (104 μL, 0.60 mmol) and 6-(trifluoromethyl)benzo[d]thiazol-2-amine (109 mg, 0.50 mmol) were added. The mixture was stirred at rt for 48 hours before silica gel was added to quench the reaction. Solvent was evaporated and the resulting residue was purified via silica gel column chromatography to yield 5-chloro-2-methoxy-3-(pyridin-3-yl)-N-(6-(trifluoromethyl)benzo[d]thiazol-2-yl)benzamide (20 mg, 10%) as a yellow solid. ¹H NMR (300 MHz, acetone) δ 8.88 (s, 1H), 8.59 (s, 1H), 8.40 (s, 1H), 8.22 (s, 1H), 8.11 (d, J=7.7 Hz, 1H), 7.85 (dd, J=17.8, 9.1 Hz, 2H), 7.63 (s, 1H), 7.60-7.46 (m, 1H), 3.28 (s, 3H). MS (ESI) [M+H]⁺ requires m/z 464.05, found m/z 464.20.

At −78° C., To a stirred solution of 5-chloro-2-methoxy-3-(pyridin-3-yl)-N-(6-(trifluoromethyl)benzo[d]thiazol-2-yl)benzamide (20 mg, 0.043 mmol) in anhydrous DCM (5 mL) was added BBr₃ (1.0M in DCM, 129 μL) dropwise. After addition, the reaction was allowed to warm to rt slowly and the mixture was stirred at rt for 2 h. After completion of the reaction, the reaction mixture was cooled in an ice bath and MeOH and water was added to quench the reaction. The mixture was separated between DCM and water. The organic layer was washed with water, brine and dried over sodium sulfate, concentrated in vacuo. The residue was purified via silica gel column chromatography to afford 5-chloro-2-hydroxy-3-(pyridin-3-yl)-N-(6-(trifluoromethyl)benzo[d]thiazol-2-yl)benzamide (12 mg, 63%) as a yellow solid. ¹H NMR (300 MHz, acetone) S 8.88 (s, 1H), 8.59 (s, 1H), 8.40 (s, 1H), 8.22 (s, 1H), 8.11 (d, J=7.7 Hz, 1H), 7.85 (dd, J=17.8, 9.1 Hz, 2H), 7.63 (s, 1H), 7.60-7.46 (m, 1H). MS (ESI) [M+H]⁺ requires m/z 450.03, found m/z 450.10.

To a stirred solution of 5-chloro-2-hydroxy-3-(pyridin-3-yl)-N-(6-(trifluoromethyl)benzo[d]thiazol-2-yl)benzamide (12 mg, 0.0267 mmol) in THE (5 mL) was added 2.0N HCl in ether (15 μL, 0.0267 mmol). The mixture was stirred at rt for 20 mins. The resulting precipitate was filtered and washed with diethyl ether to afford 5-chloro-2-hydroxy-3-(pyridin-3-yl)-N-(6-(trifluoromethyl)benzo[d]thiazol-2-yl)benzamide hydrochloride as a yellow solid (13 mg, 100%). MS (ESI) [M+H]⁺ requires m/z 450.03, found m/z 450.10.

### Example 11

### 5-chloro-3-((cis-2,6-dimethylmorpholino)methyl)-2-hydroxy-N-(6-(trifluoromethyl)benzo[d]thiazol-2-yl)benzamide(11) and 5-chloro-3-((cis-2,6-dimethylmorpholino)methyl)-2-hydroxy-N-(6-(trifluoromethyl)benzo[d]thiazol-2-yl)benzamide hydrochloride (11A)

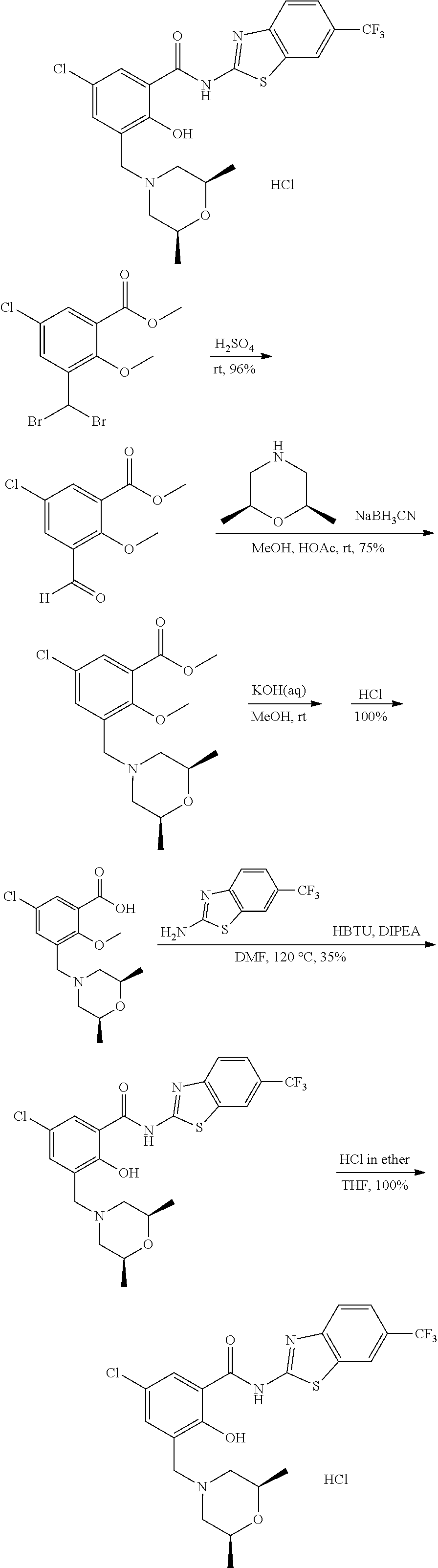

Methyl 3-(dibromomethyl)-5-chloro-2-methoxybenzoate (370 mg, 1 mmol, Example 41) was dissolved in 4 mL concentrated sulfuric acid and the mixture was stirred at rt for 2 h. The reaction mixture was poured into ice water and extracted with ethyl acetate two times. The combined organic layer was washed with brine and dried over sodium sulfate. After concentration, the residue was purified via silica gel column chromatography to give methyl 5-chloro-3-formyl-2-methoxybenzoate (220 mg, 96%) as a white solid. ¹H NMR (300 MHz, cdcl₃) δ 10.33 (s, 1H), 8.00 (d, J=2.6 Hz, 1H), 7.91 (d, J=3.1 Hz, 1H), 3.98 (s, 4H), 3.94 (s, 4H).

To a stirred solution of methyl 5-chloro-3-formyl-2-methoxybenzoate (220 mg, 0.965 mmol) in MeOH (10 mL) was added (2S,6R)-2,6-dimethylmorpholine (238 μL, 1.93 mmol), NaBH₃CN (121 mg, 1.93 mmol) and acetic acid (138 μL, 2.41 mmol). The resulting mixture was stirred at rt overnight. Saturated NaHCO₃ was added and extracted with DCM two times. The combined organic layer was concentrated in vacuo and the residue was purified via silica gel column chromatography to yield methyl 5-chloro-3-((cis-2,6-dimethylmorpholino)methyl)-2-methoxybenzoate as a yellow oil (232 mg, 75% yield). MS (ESI) [M+H]⁺ requires m/z 328.20, found m/z 328.40.

To a stirred solution of methyl 5-chloro-3-((cis-2,6-dimethylmorpholino)methyl)-2-methoxybenzoate (232 mg, 0.71 mmol) in MeOH (4 mL) was added 3.5 mL 1N KOH solution. The resulting mixture was stirred at rt overnight. The solvent was evaporated out and 4N HCl in dioxane (2 mL) was added to the residue. The mixture was stirred for another 10 minutes before it was concentrated and dried under vacumn. To this residue was added HBTU (322 mg, 0.852 mmol), DMF (5 mL) and DIPEA (618 μL, 3.55 mmol). The mixture was stirred for 10 minutes and then 6-(trifluoromethyl)benzo[d]thiazol-2-amine (154 mg, 0.71 mmol) was added. The resulting reaction was heated at 120° C. for 24 h. After cooling to rt, the mixture was separated between ethyl acetate and water. The organic layer was washed with brine, dried over sodium sulfate and concentrated in vacuo. Purification by column chromatography gave 5-chloro-3-((cis-2,6-dimethylmorpholino)methyl)-2-hydroxy-N-(6-(trifluoromethyl)benzo[d]thiazol-2-yl)benzamide as a white solid (77 mg, 35%). ¹H NMR (300 MHz, acetone) δ 8.41 (s, 1H), 8.00-7.90 (m, 2H), 7.76 (dd, J=8.5, 2.4 Hz, 1H), 7.36-7.29 (m, 1H), 4.27 (s, 2H), 4.02-3.93 (m, 2H), 3.39-3.33 (m, 4H), 2.51 (t, J=11.4 Hz, 2H), 1.23 (d, J=6.3 Hz, 6H). MS (ESI) [M+H]⁺ requires m/z 500.10, found m/z 500.30.

To a stirred solution of 5-chloro-3-((cis-2,6-dimethylmorpholino)methyl)-2-hydroxy-N-(6-(trifluoromethyl)benzo[d]thiazol-2-yl)benzamide (77 mg, 0.154 mmol) in THF (5 mL) was added 2.0N HCl in ether (77 μL, 0.154 mmol). The mixture was stirred at rt for 20 minutes. The resulting precipitate was filtered and washed with diethyl ether to afford -chloro-3-((cis-2,6-dimethylmorpholino)methyl)-2-hydroxy-N-(6-(trifluoromethyl)benzo[d]thiazol-2-yl)benzamide hydrochloride as a yellow solid (82 mg, 100%). MS (ESI) [M+H]⁺ requires m/z 500.10, found m/z 500.30.

### Example 12

### 5-chloro-2-hydroxy-3-(thiazol-2-yl)-N-(6-(trifluoromethyl)benzo[d]thiazol-2-yl)benzamide(12) and 5-chloro-2-hydroxy-3-(thiazol-2-yl)-N-(6-(trifluoromethyl)benzo[d]thiazol-2-yl)benzamide hydrochloride (12A)

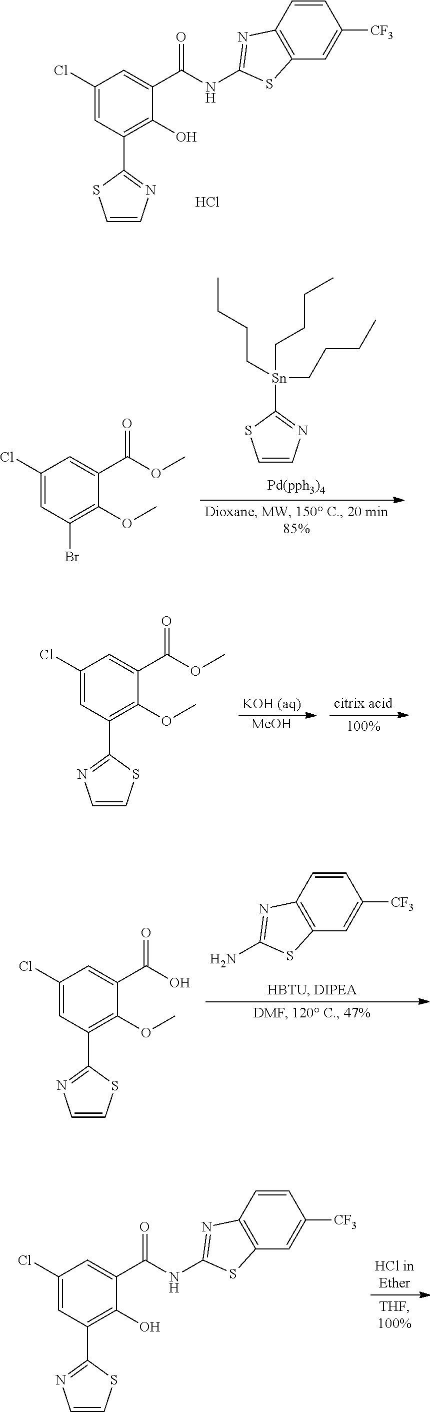

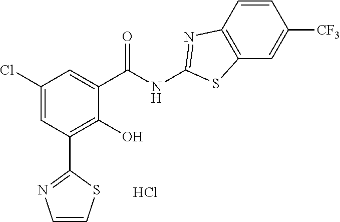 [SMILES: O=C(Nc1nc2ccc(C(F)(F)F)cc2s1)c1cc(Cl)cc(-c2nccs2)c1O ; Cl]

A mixture of methyl 3-bromo-5-chloro-2-methoxybenzoate (277 mg, 0.99 mmol, Example 2), 2-tributylstannylthiazole (346 μL, 1.1 mmol) and Pd(PPh₃)₄ (35 mg, 0.03 mmol) in dioxane (2 mL) was heated to 150° C. for 20 minutes in a microwave under nitrogen. After cooling to rt, the mixture was filtered through celite, washed with ethyl acetate and concentrated in vacuo. The resulting solid was purified by flash column to obtain methyl 5-chloro-2-methoxy-3-(thiazol-2-yl)benzoate (237 mg, 85%) as a yellow solid. MS (ESI) [M+H]⁺ requires m/z 284.01, found m/z 284.0.

To a stirred solution of methyl 5-chloro-2-methoxy-3-(thiazol-2-yl)benzoate (237 mg, 0.837 mmol) in MeOH (5 mL) and THF (4 mL) was added 4.0 mL 1N KOH solution. The resulting mixture was stirred at rt overnight. The solvent was evaporated out and 10% citric acid was added to the residue to pH=3. The mixture was stirred for another 10 minutes before it was concentrated and dried under vacumn. To this residue was added HBTU (379 mg, 1.0 mmol), DMF (5 mL) and DIPEA (729 μL, 4.185 mmol). The mixture was stirred for 10 minutess and then 6-(trifluoromethyl)benzo[d]thiazol-2-amine (182 mg, 0.837 mmol) was added. The resulting reaction was heated at 120° C. for 24 h. After cooling to rt, the mixture was separated between ethyl acetate and water. The organic layer was washed with brine, dried over sodium sulfate and concentrated in vacuo. Purification by column chromatography gave 5-chloro-2-hydroxy-3-(thiazol-2-yl)-N-(6-(trifluoromethyl)benzo[d]thiazol-2-yl)benzamide as a yellow solid (177 mg, 47%). ¹H NMR (300 MHz, cdcl₃) δ 8.28 (d, J=2.6 Hz, 1H), 8.07 (s, 1H), 7.88-7.74 (m, 3H), 7.62 (d, J=8.5 Hz, 1H), 7.42 (d, J=3.4 Hz, 1H). MS (ESI) [M+H]⁺ requires m/z 455.99, found m/z 456.2.

To a stirred solution of 5-chloro-2-hydroxy-3-(thiazol-2-yl)-N-(6-(trifluoromethyl)benzo[d]thiazol-2-yl)benzamide (177 mg, 0.389 mmol) in THE (5 mL) was added 2N HCl in ether (195 μL, 0.389 mmol). The mixture was stirred at rt for 20 minutes. The resulting precipitate was filtered and washed with diethyl ether to afford 5-chloro-2-hydroxy-3-(thiazol-2-yl)-N-(6-(trifluoromethyl)benzo[d]thiazol-2-yl)benzamide hydrochloride as a yellow solid (191 mg, 100%). MS (ESI) [M+H]⁺ requires m/z 455.99, found m/z 456.2.

### Example 13

### 5-chloro-2-hydroxy-N-(4-((2-methoxyethoxy)methyl)-6-(trifluoromethyl)benzo[d]thiazol-2-yl)-3-methylbenzamide (13)

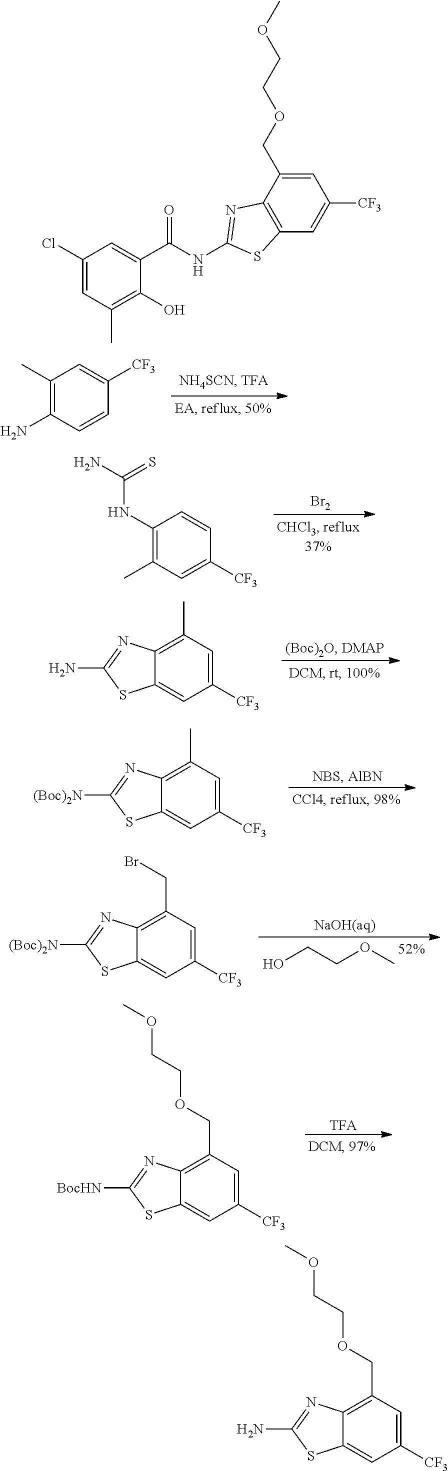

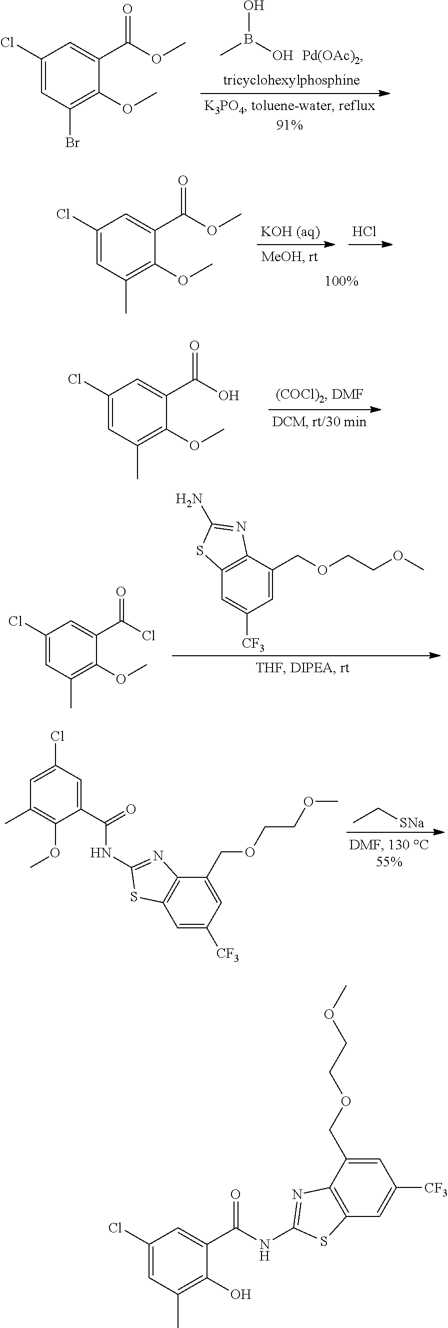

A mixture of 2-methyl-4-(trifluoromethyl)aniline (525 mg, 3 mmol), NH₄SCN (228 mg, 3 mmol) and TFA (574 μL, 7.5 mmol) in ethyl acetate (5 mL) was refluxed overnight. After cooling to rt, the mixture was partitioned between water and ethyl acetate. The ethyl acetate layer was dried over sodium sulfate and concentrated under reduced pressure. The residue was purified via silica gel column chromatography to give 1-(2-methyl-4-(trifluoromethyl)phenyl)thiourea (347 mg, 50%) as a yellow solid. MS (ESI) [M+H]⁺ requires m/z 235.05, found m/z 235.30.

At 0° C., a solution of Br₂ (76 μL, 1.48 mmol) in CHCl₃ (2 mL) was added dropwise to a stirred solution of 1-(2-methyl-4-(trifluoromethyl)phenyl)thiourea (347 mg, 1.48 mmol) in CHCl₃ (5 mL). After completion of the addition, the mixture was heated to reflux overnight. After cooling to rt, the precipitated out white solid was filtered and collected, neutralized by addition ammonia and further extracted with ethyl acetate. The organic layer was dried over sodium sulfate and concentrated under reduced pressure to afford 4-methyl-6-(trifluoromethyl)benzo[d]thiazol-2-amine (125 mg, 37%) as a white solid which was used in the next step without further purification. MS (ESI) [M+H]⁺ requires m/z 233.04, found m/z 233.20.

To a stirred solution of 4-methyl-6-(trifluoromethyl)benzo[d]thiazol-2-amine (97 mg, 0.418 mmol) and (Boc)₂O (262 mg, 1.2 mmol) in anhydrous DCM (4 mL) was added DMAP (5 mg, 0.0418 mmol). The resulting mixture was stirred at rt for 5 h. After completion of the reaction, ethyl acetate was added and washed sequentially with saturated NH₄Cl, water, saturated sodium bicarbonate and brine. The organic layer was dried over sodium sulfate and concentrated under reduced pressure to afford the tert-butyl N-tert-butoxycarbonyl-N-(4-(methyl)-6-(trifluoromethyl)benzo[d]thiazol-2-yl)carbamate (180 mg, 100%) as a yellow solid which was used in the next step without further purification. MS (ESI) [M+H]⁺ requires m/z 433.14, found m/z 433.4.

A mixture of tert-butyl N-tert-butoxycarbonyl-N-(4-(methyl)-6-(trifluoromethyl)benzo[d]thiazol-2-yl)carbamate (180 mg, 0.417 mmol), NBS (82 mg, 0.459 mmol) and AIBN (7 mg, 0.0417 mmol) in CCl₄ (5 mL) was refluxed for 2.5 h under nitrogen. After cooling to rt, the mixture was concentrated and the residue was subjected to chromatography to afford tert-butyl N-(4-(bromomethyl)-6-(trifluoromethyl)benzo[d]thiazol-2-yl)-N-tert-butoxycarbonylcarbamate (210 mg, 98%) as a yellow solid. MS (ESI) [M+H]⁺ requires m/z 511.05, found m/z 511.20.

To a stirred solution of tert-butyl N-(4-(bromomethyl)-6-(trifluoromethyl)benzo[d]thiazol-2-yl)-N-tert-butoxycarbonylcarbamate (210 mg, 0.411 mmol) in 2-methoxyethan-1-ol (8 mL) was added 1N NaOH (4 mL). The resulting mixture was stirred at rt for 30 minutes. After completion of the reaction, the mixture was partitioned between ethyl acetate and water. The ethyl acetate layer was dried over sodium sulfate and concentrated under reduced pressure. The residue was purified via silica gel column chromatography to give tert-butyl (4-((2-methoxyethoxy)methyl)-6-(trifluoromethyl)benzo[d]thiazol-2-yl)carbamate (86 mg, 52%) as a colorless oil. MS (ESI) [M+H]⁺ requires m/z 407.13, found m/z 407.3.

To a stirred solution of tert-butyl (4-((2-methoxyethoxy)methyl)-6-(trifluoromethyl)benzo[d]thiazol-2-yl)carbamate (86 mg, 0.212 mmol) in DCM (4 mL) was added TFA (3 mL), the resulting mixture was stirred at rt for 2 h. After completion of the reaction, the excess of TFA was evaporated out and the residue was partitioned between ethyl acetate and saturated sodium bicarbonate. The ethyl acetate layer was dried over sodium sulfate and concentrated under reduced pressure to afford 4-((2-methoxyethoxy)methyl)-6-(trifluoromethyl)benzo[d]thiazol-2-amine as a pale yellow solid (62 mg, 97%) which was used in the subsequent step without further purification. MS (ESI) [M+H]⁺ requires m/z 307.07, found m/z 307.20.

A mixture of methyl 3-bromo-5-chloro-2-methoxybenzoate (756 mg, 2.7 mmol, Example 2), methylboronic acid (324 mg, 5.4 mmol), Pd(OAc)₂ (24 mg, 0.11 mmol), tricyclohexylphosphine (68 mg, 0.24 mmol) and potassium phosphate tribasic (1.9 g, 8.96 mmol) was refluxed in toluene(10 mL) and water (1 mL) under N₂ overnight. After the reaction was cooled, saturated NH₄Cl solution was added and extracted with ethyl acetate two times. The combined ethyl acetate layer was dried over sodium sulfate and concentrated under reduced pressure. The residue was purified via silica gel column chromatography to give methyl 5-chloro-2-methoxy-3-methylbenzoate (520 mg, 91%) as a yellow oil. ¹H NMR (300 MHz, chloroform) δ 7.62 (d, 1H, J=3.0 Hz), 7.32 (d, 1H, J=3.0 Hz), 3.92 (s, 3H), 3.82 (s, 3H), 2.30 (s, 3H).

To a stirred solution of methyl 5-chloro-2-methoxy-3-methylbenzoate (43 mg, 0.20 mmol) in MeOH (5 mL) was added 1.0 mL 1N KOH solution. The resulting mixture was stirred at rt overnight. After completion of the reaction, 1N HCl was added to the reaction and extracted with ethyl acetate two times. The combined organic phase was dried over sodium sulfate and concentrated under reduced pressure to afford 5-chloro-2-methoxy-3-methylbenzoic acid as a residue (white solid, 40 mg, 100%) which was used in the next step without further purification.

5-chloro-2-methoxy-3-methylbenzoic acid (40 mg, 0.20 mmol) was dissolved in DCM (3.0 mL), followed by the addition of acatalytic amount of DMF (1 drop) and oxalyl chloride (21 μL, 0.243 mmol) respectively. The reaction was allowed to stir at rt for 30 minutes and concentrated in vacuo. The residue was re-dissolved in THF (5.0 mL), and Hunig's base (43 μL, 0.243 mmol) and 4-((2-methoxyethoxy)methyl)-6-(trifluoromethyl)benzo[d]thiazol-2-amine (62 mg, 0.20 mmol) were added. The mixture was stirred at rt for 48 hours before silica gel was added to quench the reaction. Solvent was evaporated and the resulting residue was purified via silica gel column chromatography to yield 5-chloro-2-methoxy-N-(4-((2-methoxyethoxy)methyl)-6-(trifluoromethyl)benzo[d]thiazol-2-yl)-3-methylbenzamide (20 mg, 21%) as a white solid. MS (ESI) [M+H]⁺ requires m/z 489.08, found m/z 489.20.

A solution of 5-chloro-2-methoxy-N-(4-((2-methoxyethoxy)methyl)-6-(trifluoromethyl)benzo[d]thiazol-2-yl)-3-methylbenzamide (20 mg, 0.04 mmol) in DMF (5 mL) is mixed with sodium ethanethiolate (17 mg, 0.205 mmol) and the resulting suspension is heated at 130° C. overnight. After completion of the reaction, 1N HCl was added to the reaction and extracted with ethyl acetate two times. The combined ethyl acetate layer was dried over sodium sulfate and concentrated under reduced pressure. The residue was purified via silica gel column chromatography to give the 5-chloro-2-hydroxy-N-(4-((2-methoxyethoxy)methyl)-6-(trifluoromethyl)benzo[d]thiazol-2-yl)-3-methylbenzamide (15 mg, 55%) as a white solid. ¹H NMR (300 MHz, cdcl₃) δ 7.95 (s, 2H), 7.58 (s, 1H), 7.29 (s, 1H), 4.96 (s, 2H), 3.98-3.86 (m, 2H), 3.84-3.73 (m, 2H), 3.64 (s, 3H), 2.28 (s, 3H). MS (ESI) [M+H]⁺ requires m/z 475.06, found m/z 475.20.

### Example 14

### 5-chloro-2-hydroxy-3-(tetrahydrofuran-3-yl)-N-(6-(trifluoromethyl)benzo[d]thiazol-2-yl)benzamide (14)

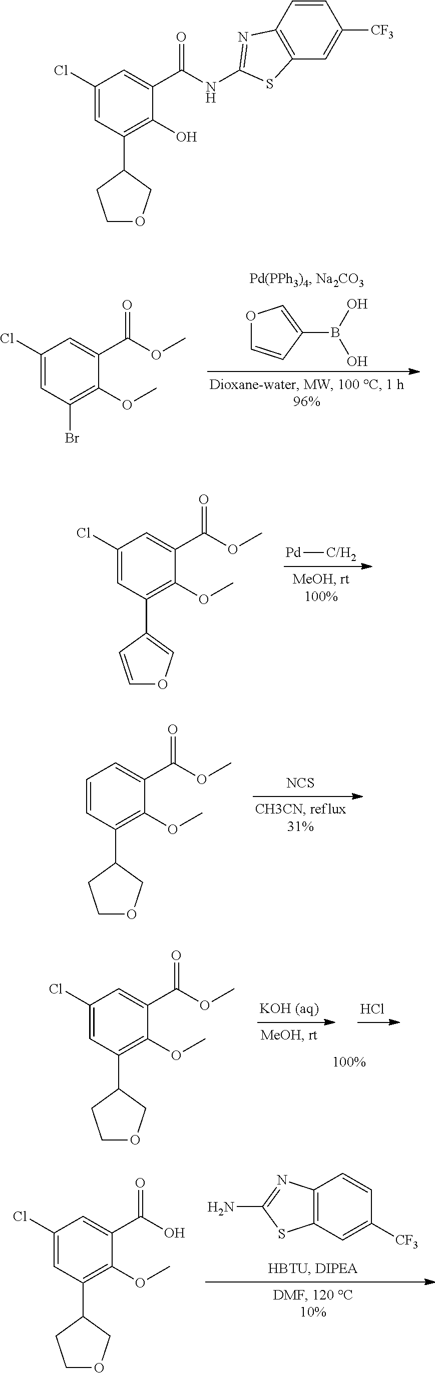

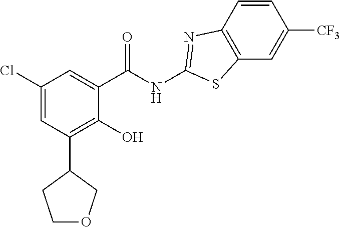 [SMILES: O=C(Nc1nc2ccc(C(F)(F)F)cc2s1)c1cc(Cl)cc(C2CCOC2)c1O]

A mixture of methyl 3-bromo-5-chloro-2-methoxybenzoate (275 mg, 0.98 mmol, Example 2), furan-3-ylboronic acid (218 mg, 1.96 mmol), Pd(PPh₃)₄ (56 mg, 0.049 mmol) and sodium carbonate (312 mg, 2.95 mmol) in dioxane (3 mL) and water (1 mL) was irradiated under microwave at 100° C. for 1 h under nitrogen. After cooling to rt, the mixture was partitioned between water and ethyl acetate. The ethyl acetate layer was dried over sodium sulfate and concentrated under reduced pressure. The residue was purified via silica gel column chromatography to give methyl 5-chloro-3-(furan-3-yl)-2-methoxybenzoate (250 mg, 96%) as a colorless oil. MS (ESI) [M+H]⁺ requires m/z 267.03, found m/z 267.20.

A mixture of methyl 5-chloro-3-(furan-3-yl)-2-methoxybenzoate (267 mg, 1.0 mmol) and Pd—C(27 mg) in MeOH (5 mL) was stirred at rt under hydrogen atmosphere for 5 h. The reaction mixture was filtered and the filtrate was concentrated to afford methyl 2-methoxy-3-(tetrahydrofuran-3-yl)benzoate (238 mg, 100%) as a colorless oil, which was used directly in the next step without further purification. MS (ESI) [M+H]⁺ requires m/z 237.1, found m/z 237.3.

A mixture of methyl 2-methoxy-3-(tetrahydrofuran-3-yl)benzoate (238 mg, 1 mmol) and NCS (134 mg, 1.0 mmol) in acetonitrile (5 mL) was refluxed for 24 h. After cooling to rt, the solvent was concentrated and the residue was subjected to column directly to afford methyl 5-chloro-2-methoxy-3-(tetrahydrofuran-3-yl)benzoate (85 mg, 31%) as a colorless oil. ¹H NMR (300 MHz, cdcl₃) δ 7.66-7.60 (m, 1H), 7.42 (d, J=2.7 Hz, 1H), 4.11-4.03 (m, 2H), 3.90 (s, 3H), 3.86-3.74 (m, 5H), 3.72-3.64 (m, 1H), 2.44-2.29 (m, 1H), 1.98-1.84 (m, 1H). MS (ESI) [M+H]⁺ requires m/z 271.07, found m/z 271.2.

To a stirred solution of 5-chloro-2-methoxy-3-(tetrahydrofuran-3-yl)benzoate (85 mg, 0.315 mmol) in MeOH (5 mL) was added 2.0 mL 1N KOH solution. The resulting mixture was stirred at rt overnight. The solvent was evaporated out and 4N HCl in dioxane (1 mL) was added to the residue. The mixture was stirred for another 10 minutes before it was concentrated and dried under vacumn. To this residue was added HBTU (143 mg, 0.378 mmol), DMF (5 mL) and DIPEA (274 μL, 1.575 mmol). The mixture was stirred for 10 minutes and then 6-(trifluoromethyl)benzo[d]thiazol-2-amine (68 mg, 0.315 mmol) was added. The resulting reaction was heated at 120° C. for 24 h. After cooling to rt, the mixture was separated between ethyl acetate and water. The organic layer was washed with brine, dried over sodium sulfate and concentrated in vacuo. Purification by column chromatography gave 5-chloro-2-hydroxy-3-(tetrahydrofuran-3-yl)-N-(6-(trifluoromethyl)benzo[d]thiazol-2-yl)benzamide as a white solid (14 mg, 10%). ¹H NMR (400 MHz, acetone) δ 8.42 (s, 1H), 8.12 (d, J=2.6 Hz, 1H), 7.91 (d, J=8.5 Hz, 1H), 7.83 (dd, J=8.5, 1.4 Hz, 1H), 7.47 (d, J=2.6 Hz, 1H), 4.09-3.96 (m, 2H), 3.88-3.71 (m, 3H), 2.37-2.31 (m, 1H), 1.98-1.84 (m, 1H). MS (ESI) [M+H]⁺ requires m/z 443.04, found m/z 443.3.

### Example 15

### 5-chloro-2-hydroxy-3-(tetrahydrofuran-2-yl)-N-(6-(trifluoromethyl)benzo[d]thiazol-2-yl)benzamide (15)

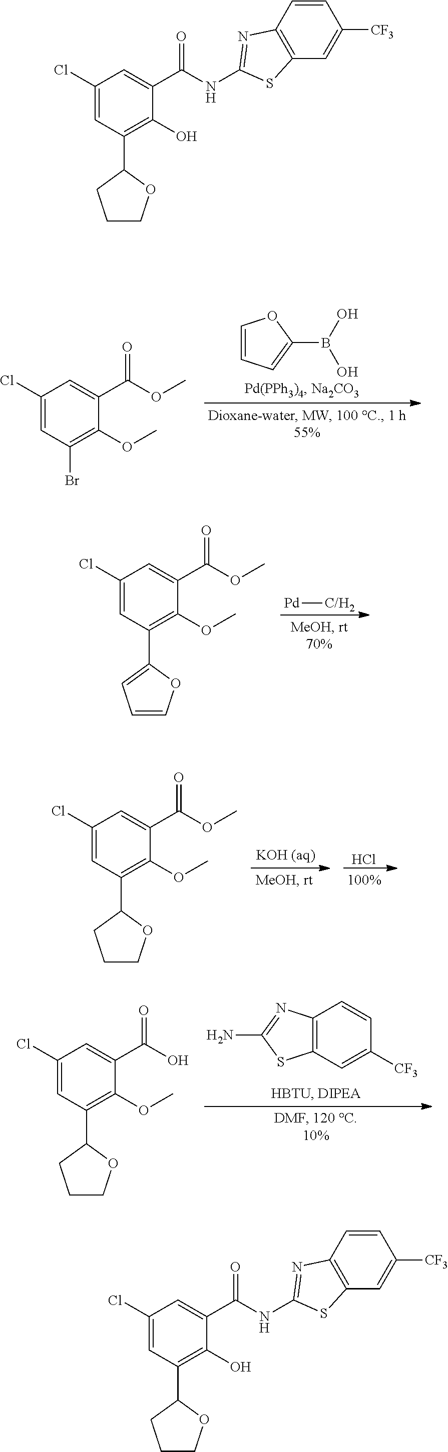

A mixture of methyl 3-bromo-5-chloro-2-methoxybenzoate (253 mg, 0.90 mmol, Example 2), furan-2-ylboronic acid (132 mg, 1.17 mmol), Pd(PPh₃)₄ (52 mg, 0.045 mmol) and sodium carbonate (287 mg, 2.7 mmol) in dioxane (3 mL) and water (1 mL) was irradiated under microwave at 100° C. for 1 h under nitrogen. After cooling to rt, the mixture was partitioned between water and ethyl acetate. The ethyl acetate layer was dried over sodium sulfate and concentrated under reduced pressure. The residue was purified via silica gel column chromatography to give methyl 5-chloro-3-(furan-2-yl)-2-methoxybenzoate (131 mg, 55%) as a colorless oil. MS (ESI) [M+H]⁺ requires m/z 267.03, found m/z 267.20.

A mixture of methyl 5-chloro-3-(furan-2-yl)-2-methoxybenzoate (131 mg, 0.5 mmol) and Pd—C(15 mg) in MeOH (5 mL) was stirred at rt under hydrogen atmosphere for 6 h. The reaction mixture was filtered and the filtrate was concentrated, the residue was purified via silica gel column chromatography to give methyl 5-chloro-2-methoxy-3-(tetrahydrofuran-2-yl)benzoate (95 mg, 70%) as a colorless oil. ¹H NMR (300 MHz, cdcl₃) δ 7.71 (d, J=2.8 Hz, 1H), 7.62 (dd, J=2.8, 0.6 Hz, 1H), 5.13 (t, J=7.2 Hz, 1H), 4.16-4.08 (m, 1H), 3.99-3.90 (m, 1H), 3.93 (s, 3H), 3.84 (s, 3H), 2.52-2.38 (m, 1H), 2.07-1.96 (m, 2H), 1.73-1.65 (m, 1H). MS (ESI) [M+H]⁺ requires m/z 271.07, found m/z 271.2.

To a stirred solution of 5-chloro-2-methoxy-3-(tetrahydrofuran-2-yl)benzoate (95 mg, 0.352 mmol) in MeOH (5 mL) was added 2.0 mL 1N KOH solution. The resulting mixture was stirred at rt overnight. The solvent was evaporated out and 4N HCl in dioxane (1 mL) was added to the residue. The mixture was stirred for another 10 minutes before it was concentrated and dried under vacumn. To this residue was added HBTU (160 mg, 0.422 mmol), DMF (5 mL) and DIPEA (306 μL, 1.76 mmol). The mixture was stirred for 10 minutes and then 6-(trifluoromethyl)benzo[d]thiazol-2-amine (76 mg, 0.352 mmol) was added. The resulting reaction was heated at 120° C. for 24 h. After cooling to rt, the mixture was separated between ethyl acetate and water. The organic layer was washed with brine, dried over sodium sulfate and concentrated in vacuo. Purification by column chromatography gave 5-chloro-2-hydroxy-3-(tetrahydrofuran-2-yl)-N-(6-(trifluoromethyl)benzo[d]thiazol-2-yl)benzamide as a white solid (15 mg, 10%). ¹H NMR (500 MHz, acetone) δ 8.42 (s, 1H), 8.11 (s, 1H), 7.91 (d, J=8.3 Hz, 1H), 7.82 (d, J=8.5 Hz, 1H), 7.56 (s, 1H), 5.12 (t, J=7.1 Hz, 1H), 4.17-4.10 (m, 1H), 3.94-3.85 (m, 1H), 2.55-2.45 (m, 1H), 2.03-1.91 (m, 2H), 1.78-1.66 (m, 1H). MS (ESI) [M+H]⁺ requires m/z 443.04, found m/z 443.3.

### Example 16

### 5-chloro-2-hydroxy-3-(morpholinomethyl)-N-(6-(trifluoromethyl)benzo[d]thiazol-2-yl)benzamide(16) and 5-chloro-2-hydroxy-3-(morpholinomethyl)-N-(6-(trifluoromethyl)benzo[d]thiazol-2-yl)benzamide hydrochloride (16A)

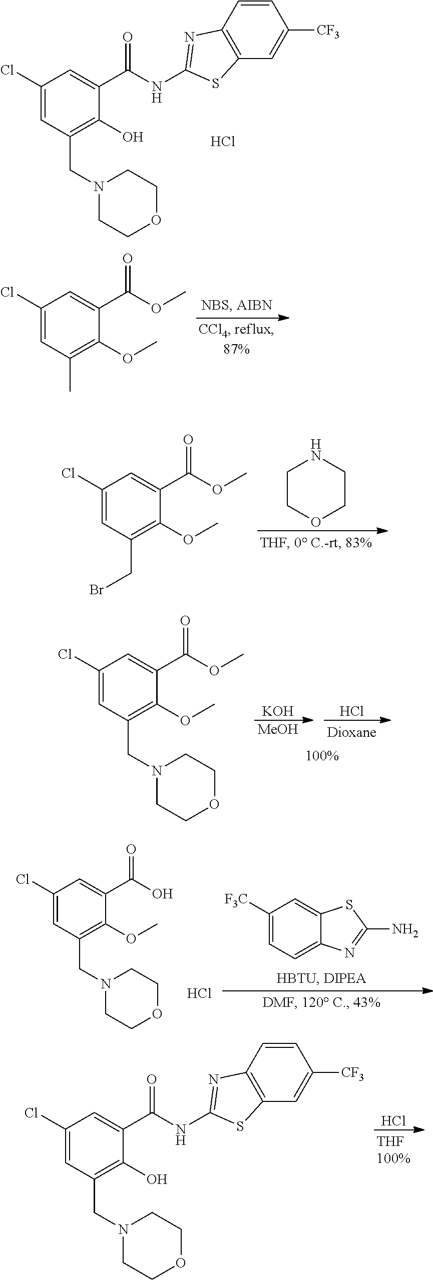

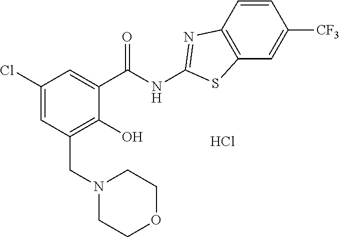 [SMILES: O=C(Nc1nc2ccc(C(F)(F)F)cc2s1)c1cc(Cl)cc(CN2CCOCC2)c1O ; Cl]

To a flame dried flask was added NBS (183 mg, 1.03 mmol), AIBN (15 mg, 0.093 mmol) and a solution of methyl 5-chloro-2-methoxy-3-methylbenzoate (201 g, 0.93 mmol) in CCl₄ (10 mL). The suspension was refluxed in the dark overnight. The mixture was cooled to rt and concentrated. The residue was purified via silica gel column chromatography to give methyl 5-chloro-3-(bromomethyl)-2-methoxybenzoate as a colorless oil (236 mg, 87%). ¹H NMR (300 MHz, cdcl₃) δ 7.76 (d, J=2.7 Hz, 1H), 7.54 (d, J=2.7 Hz, 1H), 4.51 (s, 2H), 3.96 (s, 3H), 3.94 (s, 3H).

At 0° C., to a stirred solution of methyl 5-chloro-3-(bromomethyl)-2-methoxybenzoate (132 mg, 0.45 mmol) in THF (4 mL) was added morpholine (78 μL, 0.90 mmol) and the mixture was stirred at rt for 6 h. After completion of the reaction, the reaction was partitioned between DCM and water. The organic layer was dried over sodium sulfate and concentrated under reduced pressure. The residue was purified by flash column to afford methyl 5-chloro-2-methoxy-3-(morpholinomethyl)benzoate (110 mg, 83%) as a colorless oil. ¹H NMR (500 MHz, cdcl₃) δ 7.68 (d, J=2.8 Hz, 1H), 7.58 (d, J=2.7 Hz, 1H), 3.90 (s, 3H), 3.82 (s, 3H), 3.70 (t, 4H), 3.52 (s, 2H), 2.47 (t, J=4.1 Hz, 4H). MS (ESI) [M+H]⁺ requires m/z 300.10, found m/z 300.30.

To a stirred solution of methyl 5-chloro-2-methoxy-3-(morpholinomethyl)benzoate (110 mg, 0.368 mmol) in MeOH (3 mL) was added 2.0 mL 1N KOH solution. The resulting mixture was stirred at rt overnight. The solvent was evaporated out and 4N HCl in dioxane (1 mL) was added to the residue. The mixture was stirred for another 10 minutes before it was concentrated and dried under vacumn. To this residue was added HBTU (168 mg, 0.442 mmol), DMF (5 mL) and DIPEA (320 μL, 1.84 mmol). The mixture was stirred for 10 minutes and then 6-(trifluoromethyl)benzo[d]thiazol-2-amine (80 mg, 0.368 mmol) was added. The resulting reaction mixture was heated at 120° C. for 24 h. After cooling to rt, the mixture was separated between ethyl acetate and water. The organic layer was washed with brine, dried over sodium sulfate and concentrated in vacuo. Purification by column chromatography gave 5-chloro-2-hydroxy-3-(morpholinomethyl)-N-(6-(trifluoromethyl)benzo[d]thiazol-2-yl)benzamide (75 mg, 43%) as a yellow solid. ¹H NMR (400 MHz, dmso) δ 8.39 (s, 1H), 7.80 (dd, J=9.5, 5.7 Hz, 2H), 7.67 (d, J=8.6 Hz, 1H), 7.37 (d, J=2.9 Hz, 1H), 4.23 (s, 2H), 3.81 (brs, 4H), 3.17 (brs, 4H). MS (ESI) [M+H]⁺ requires m/z 472.07, found m/z 472.20.

To a stirred solution of 5-chloro-2-hydroxy-3-(morpholinomethyl)-N-(6-(trifluoromethyl)benzo[d]thiazol-2-yl)benzamide (32 mg, 0.068 mmol) in THE (5 mL) was added 4.0N HCl in dioxane (20 μL, 0.075 mmol). The mixture was stirred at rt for 20 minutes. The resulting precipitate was filtered and washed with diethyl ether to afford 5-chloro-2-hydroxy-3-(morpholinomethyl)-N-(6-(trifluoromethyl)benzo[d]thiazol-2-yl)benzamide hydrochloride as a yellow solid (100%). MS (ESI) [M+H]⁺ requires m/z 472.07, found m/z 472.20.

### Example 17

### 5-chloro-2-hydroxy-3-(2-morpholinoethyl)-N-(6-(trifluoromethyl)benzo[d]thiazol-2-yl)benzamide(17) and 5-chloro-2-hydroxy-3-(2-morpholinoethyl)-N-(6-(trifluoromethyl)benzo[d]thiazol-2-yl)benzamide hydrochloride (17A)

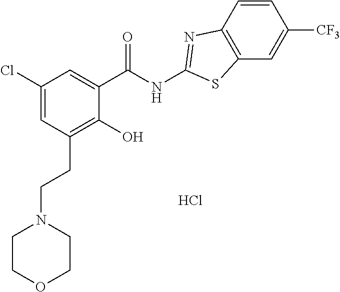 [SMILES: O=C(Nc1nc2ccc(C(F)(F)F)cc2s1)c1cc(Cl)cc(CCN2CCOCC2)c1O ; Cl]

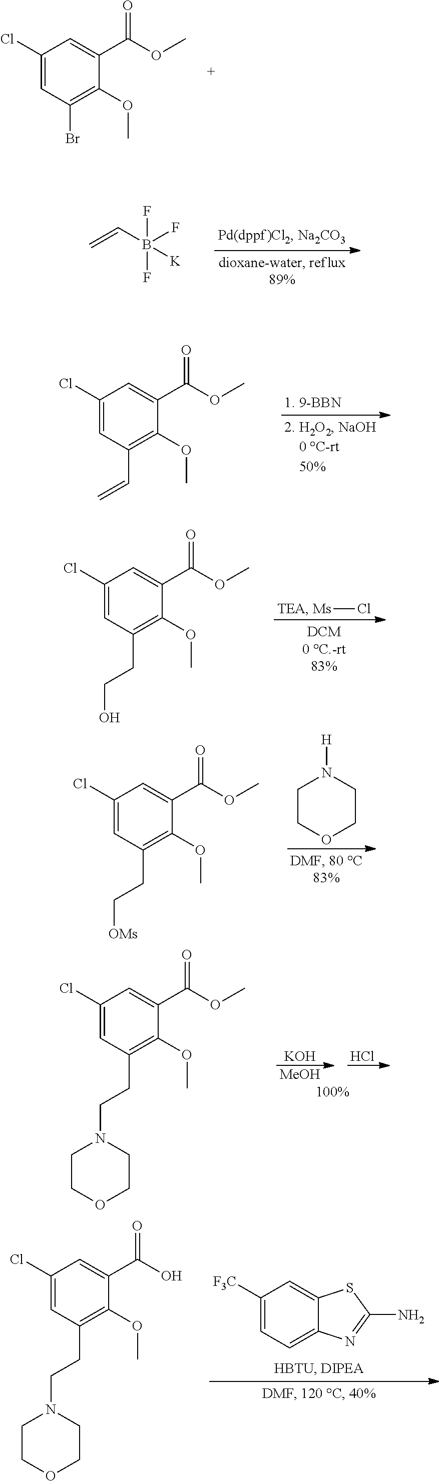

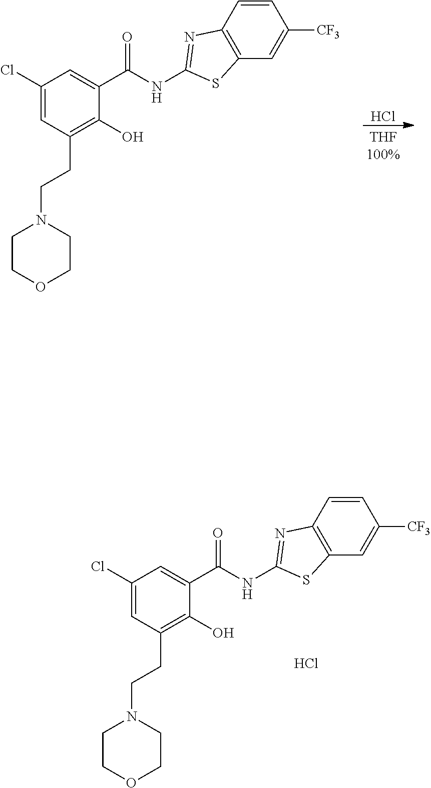 [SMILES: O=C(Nc1nc2ccc(C(F)(F)F)cc2s1)c1cc(Cl)cc(CCN2CCOCC2)c1O ; Cl ; O=C(Nc1nc2ccc(C(F)(F)F)cc2s1)c1cc(Cl)cc(CCN2CCOCC2)c1O ; Cl]

A mixture of methyl 3-bromo-5-chloro-2-methoxybenzoate (445 mg, 1.6 mmol, Example 2), potassium trifluoro(vinyl)borate (322 mg, 2.4 mmol), Pd(dppf)Cl₂, (40 mg, 0.05 mmol), and sodium carbonate (339 mg, 3.2 mmol) was refluxed in dioxane (10 mL) and water (1 mL) under N₂ overnight. After the reaction was cooled, saturated NH₄Cl solution was added and extracted with ethyl acetate two times. The combined ethyl acetate layer was dried over sodium sulfate and concentrated under reduced pressure. The residue was purified via silica gel column chromatography to give methyl 5-chloro-2-methoxy-3-vinylbenzoate (320 mg, 89%) as a colorless oil. ¹H NMR (300 MHz, cdcl₃) δ 7.69 (d, J=2.7 Hz, 1H), 7.63 (d, J=2.7 Hz, 1H), 6.99 (dd, J=17.7, 11.1 Hz, 1H), 5.79 (dd, J=17.7, 0.8 Hz, 1H), 5.43 (dd, J=11.1, 0.8 Hz, 1H), 3.93 (s, 3H), 3.83 (s, 3H).

At 0° C., to a stirred solution of methyl 5-chloro-2-methoxy-3-vinylbenzoate (270 mg, 1.19 mmol) was added 9-BBN (3.1 mL, 1.55 mmol, 0.5M in THF) dropwise. After completion of the addition, the mixture was allowed to stir at rt overnight. The reaction was cooled to 0° C. and 1 mL of 50% H₂O₂ solution was added followed by 1 mL of 3M NaOH solution. The mixture was stirred over an ice bath for 2 h. The reaction was partitioned between water and ethyl acetate. The ethyl acetate layer was dried over sodium sulfate and concentrated under reduced pressure. The residue was purified via silica gel column chromatography to give methyl 5-chloro-3-(2-hydroxyethyl)-2-methoxybenzoate (110 mg, 50%) as a colorless oil. ¹H NMR (400 MHz, d₂o) δ 7.65 (s, 1H), 7.37 (s, 1H), 3.90 (s, 3H), 3.85-3.78 (m, 5H), 2.88 (t, J=6.3 Hz, 2H). MS (ESI) [M+H]⁺ requires m/z 245.06, found m/z 245.20.

At 0° C., to a stirred solution of methyl 5-chloro-3-(2-hydroxyethyl)-2-methoxybenzoate (110 mg, 0.451 mmol) in DCM (3 mL) was added TEA (95 μL, 0.677 mmol) and MsCl (42 μL, 0.54 mmol). The resulting mixture was stirred at 0° C. for 1 h. The reaction mixture was partitioned between DCM and saturated NH₄Cl solution. The organic layer was dried over sodium sulfate and concentrated under reduced pressure. The residue was purified via silica gel column chromatography to give methyl 5-chloro-2-methoxy-3-(2-((methylsulfonyl)oxy)ethyl)benzoate (119 mg, 83%) as a colorless oil. ¹H NMR (500 MHz, cdcl₃) δ 7.71 (d, J=2.7 Hz, 1H), 7.37 (d, J=2.7 Hz, 1H), 4.41 (t, J=6.8 Hz, 2H), 3.92 (s, 3H), 3.83 (s, 3H), 3.08 (t, J=6.8 Hz, 2H), 2.93 (s, 3H).

To a stirred solution of methyl 5-chloro-2-methoxy-3-(2-((methylsulfonyl)oxy)ethyl)benzoate (119 mg, 0.369 mmol) in DMF (3 mL) was added morpholine (160 μL, 1.84 mmol). The resulting mixture was stirred at 80° C. overnight. The reaction was partitioned between water and DCM. The organic layer was dried over sodium sulfate and concentrated under reduced pressure. The residue was purified via silica gel column chromatography to give methyl 5-chloro-2-methoxy-3-(2-morpholinoethyl)benzoate (91 mg, 83%) as a yellow oil. ¹H NMR (300 MHz, cdcl₃) δ 7.65 (d, J=2.3 Hz, 1H), 7.35 (d, 1H), 3.91 (s, 3H), 3.82 (s, 3H), 3.78-3.69 (m, 4H), 2.86-2.79 (m, 2H), 2.64-2.56 (m, 2H), 2.57-2.49 (m, 4H). MS (ESI) [M+H]⁺ requires m/z 314.12, found m/z 314.20.

To a stirred solution of methyl 5-chloro-2-methoxy-3-(2-morpholinoethyl)benzoate (91 mg, 0.29 mmol) in MeOH (3 mL) was added 1.5 mL 1N KOH solution. The resulting mixture was stirred at rt overnight. The solvent was evaporated out and 4N HCl in dioxane (1 mL) was added to the residue. The mixture was stirred for another 10 minutes before it was concentrated and dried under vacumn. To this residue was added HBTU (132 mg, 0.35 mmol), DMF (5 mL) and DIPEA (253 μL, 1.45 mmol). The mixture was stirred for 10 minutes and then 6-(trifluoromethyl)benzo[d]thiazol-2-amine (64 mg, 0.29 mmol) was added. The resulting reaction was heated at 120° C. for 24 h. After cooling to rt, the mixture was separated between ethyl acetate and water. The organic layer was washed with brine, dried over sodium sulfate and concentrated in vacuo. Purification by column chromatography gave 5-chloro-2-hydroxy-3-(2-morpholinoethyl)-N-(6-(trifluoromethyl)benzo[d]thiazol-2-yl)benzamide (56 mg, 40%) as a yellow solid. ¹H NMR (300 MHz, acetone) δ 8.41-8.36 (m, 1H), 7.94-7.86 (m, 2H), 7.77-7.70 (m, 1H), 7.24 (d, J=2.9 Hz, 1H), 4.04 (brs, 4H), 3.40-3.02 (m, 8H). MS (ESI) [M+H]⁺ requires m/z 486.10, found m/z 486.20.

To a stirred solution of 5-chloro-2-hydroxy-3-(2-morpholinoethyl)-N-(6-(trifluoromethyl)benzo[d]thiazol-2-yl)benzamide (36 mg, 0.074 mmol) in THE (5 mL) was added 4.0N HCl in dioxane (21 μL, 0.082 mmol). The mixture was stirred at rt for 20 minutes. The resulting precipitate was filtered and washed with diethyl ether to afford 5-chloro-2-hydroxy-3-(2-morpholinoethyl)-N-(6-(trifluoromethyl)benzo[d]thiazol-2-yl)benzamide hydrochloride as a yellow solid (100%). MS (ESI) [M+H]⁺ requires m/z 486.10, found m/z 486.20.

### Example 18

### 5-chloro-N-(2-chloro-4-(trifluoromethyl)phenyl)-3-(2-(dimethylamino)ethyl)-2-hydroxybenzamide (18)

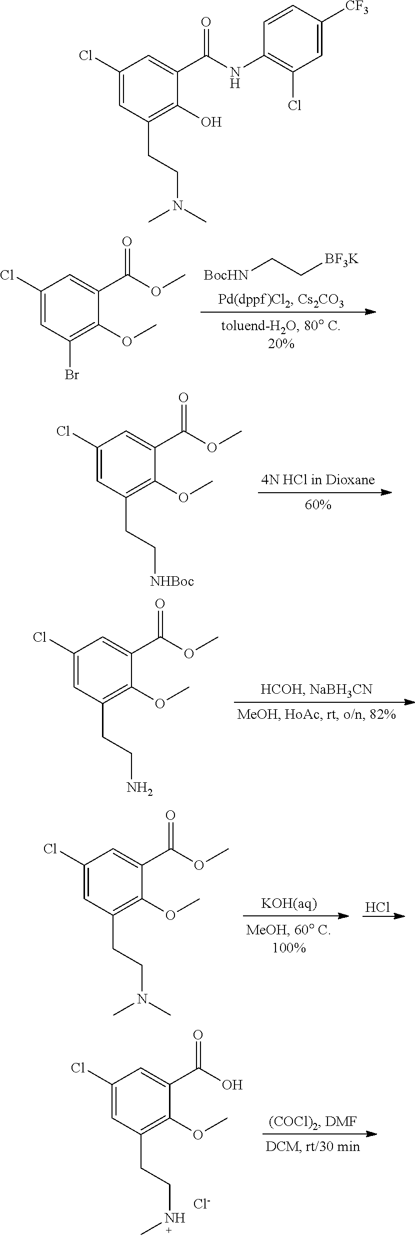

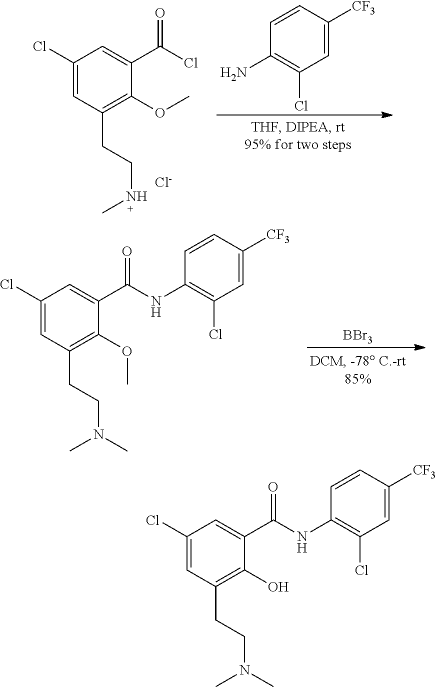

Methyl 3-bromo-5-chloro-2-methoxybenzoate (6.18 g, 22.07 mmol, Example 2), potassium (2-((tert-butoxycarbonyl)amino)ethyl)trifluoroborate (16.6 g, 66.21 mmol), Pd(dppf)Cl₂ (968 mg, 1.32 mmol) and Cs₂CO₃ (21.5 g, 66.21 mmol) were added to a flask. This flask was evacuated and refilled with N₂ three times. Subsequently, toluene (75 mL) and water (25 mL) were added to the flask under N₂. The mixture was stirred at 80° C. under N₂ overnight. Saturated NH₄Cl solution was added and the resulting mixture was extracted with ethyl acetate two times. The combined organic layer was concentrated in vacuo and the residue was purified via silica gel column chromatography to yield methyl 3-(2-((tert-butoxycarbonyl)amino)ethyl)-5-chloro-2-methoxybenzoate as a yellow oil (1.51 g, 20% yield). 1H NMR (300 MHz, cdcl3) δ 7.64 (d, J=2.7 Hz, 1H), 7.31 (d, J=2.7 Hz, 1H), 3.90 (s, 3H), 3.81 (s, 3H), 3.34 (brs, 2H), 2.82 (t, J=6.9 Hz, 2H), 1.41 (s, 9H). MS (ESI) [M+Na]⁺ requires m/z 366.11, found m/z 365.95.

Methyl 3-(2-((tert-butoxycarbonyl)amino)ethyl)-5-chloro-2-methoxybenzoate (1.12 g, 3.26 mmol) was dissolved in 4N HCl in dioxane (5 mL) and the resulting mixture was stirred at rt for 2 h. Saturated NaHCO₃ solution was added and extracted with DCM. The organic layer was washed with water and brine and dried over sodium sulfate. The organic layer was filtered and the solvent removed in vacuo to yield methyl 3-(2-aminoethyl)-5-chloro-2-methoxybenzoate as a pale yellow oil (475 mg, 60%). 1H NMR (500 MHz, cdcl3) δ 7.61 (d, J=2.7 Hz, 1H), 7.30 (d, J=2.7 Hz, 1H), 3.87 (s, 3H), 3.78 (s, 3H), 2.92 (t, J=7.5 Hz, 2H), 2.75 (t, J=7.1 Hz, 2H), 1.61 (s, 2H).). MS (ESI) [M+H]⁺ requires m/z 244.08, found m/z 244.30.

To a stirred solution of methyl 3-(2-aminoethyl)-5-chloro-2-methoxybenzoate (100 mg, 0.411 mmol) in MeOH (3 mL) was added formaldehyde (122 μL, 1.64 mmol, 37% wt in H₂O), NaBH₃CN (103 mg, 1.64 mmol) and acetic acid (117 μL, 2.06 mmol). The resulting mixture was stirred at rt overnight. Saturated NaHCO₃ was added and extracted with DCM two times. The combined organic layer was concentrated in vacuo and the residue was purified via silica gel column chromatography to yield methyl 5-chloro-3-(2-(dimethylamino)ethyl)-2-methoxybenzoate as a yellow oil (88 mg, 82% yield). ¹H NMR (300 MHz, cdcl₃) δ 7.66 (d, J=2.5 Hz, 1H), 7.36 (d, J=2.6 Hz, 1H), 3.92 (s, 3H), 3.84 (s, 3H), 2.86 (t, J=7.5 Hz, 2H), 2.58 (t, J=7.5 Hz, 2H), 2.34 (s, 6H). MS (ESI) [M+H]⁺ requires m/z 272.10, found m/z 272.05.

To a stirred solution of methyl 5-chloro-3-(2-(dimethylamino)ethyl)-2-methoxybenzoate (82 mg, 0.3 mmol) in MeOH (3 mL) was added 1N KOH (1 mL) solution. The mixture was stirred at rt overnight. 1N HCl was added to adjust the pH to 1. The mixture was concentrated in vacuo. The residue was dissolved in DCM (3 mL) followed by the addition of catalytic amount of DMF (1 drop) and oxalyl chloride (32 μL, 0.36 mmol) respectively. The reaction was allowed to stir at rt for 2 h then concentrated in vacuo. The residue was re-dissolved in THF (5.0 mL), and Hunig's base (157 μL, 0.90 mmol) and 2-chloro-4-(trifluoromethyl)aniline (42 μL, 0.30 mmol) were added. The mixture was stirred at rt for 48 hours before silica gel was added to quench the reaction. Solvent was evaporated and the resulting residue was purified via silica gel column chromatography to yield 5-chloro-N-(2-chloro-4-(trifluoromethyl)phenyl)-3-(2-(dimethylamino)ethyl)-2-methoxybenzamide (124 mg, 95%) as a white solid. ¹H NMR (300 MHz, cdcl₃) δ 10.61 (s, 1H), 8.85 (d, J=8.7 Hz, 1H), 8.02 (d, J=2.7 Hz, 1H), 7.71 (s, 1H), 7.60 (d, J=8.7 Hz, 1H), 7.45 (d, J=2.7 Hz, 1H), 3.92 (s, 3H), 3.04-2.79 (m, 2H), 2.77-2.49 (m, 2H), 2.42 (s, 6H). MS (ESI) [M+H]⁺ requires m/z 435.09, found m/z 435.0.

At −78° C., to a stirred solution of 5-chloro-N-(2-chloro-4-(trifluoromethyl)phenyl)-3-(2-(dimethylamino)ethyl)-2-methoxybenzamide (69 mg, 0.16 mmol) in anhydrous DCM (5 mL) was added BBr₃ (1.0M in DCM, 0.635 mL) dropwise. After addition, the reaction was allowed to warm to rt slowly and the mixture was stirred at rt for 2 h. After completion of the reaction, the reaction mixture was cooled in an ice bath and MeOH and water were added to quench the reaction. The mixture was separated between DCM and water. The organic layer was washed with water, brine and dried over sodium sulfate, concentrated in vacuo. The residue was purified via silica gel column chromatography to afford 5-chloro-N-(2-chloro-4-(trifluoromethyl)phenyl)-3-(2-(dimethylamino)ethyl)-2-hydroxybenzamide (56 mg, 85%) as a yellow solid. ¹H NMR (300 MHz, cdcl₃) δ 12.50 (s, 1H), 8.96-8.83 (m, 1H), 8.05 (d, J=2.8 Hz, 1H), 7.65 (d, J=1.4 Hz, 1H), 7.55 (dd, J=8.7, 1.5 Hz, 1H), 7.11 (d, J=2.8 Hz, 1H), 2.91 (s, 4H), 2.62 (s, 6H). MS (ESI) [M+H]⁺ requires m/z 421.07, found m/z 421.20.

### Example 19

### 3-((4-acetylpiperazin-1-yl)methyl)-5-chloro-2-hydroxy-N-(6-(trifluoromethyl)benzo[d]thiazol-2-yl)benzamide (19) and 3-((4-acetylpiperazin-1-yl)methyl)-5-chloro-2-hydroxy-N-(6-(trifluoromethyl)benzo[d]thiazol-2-yl)benzamide hydrochloride (19A)

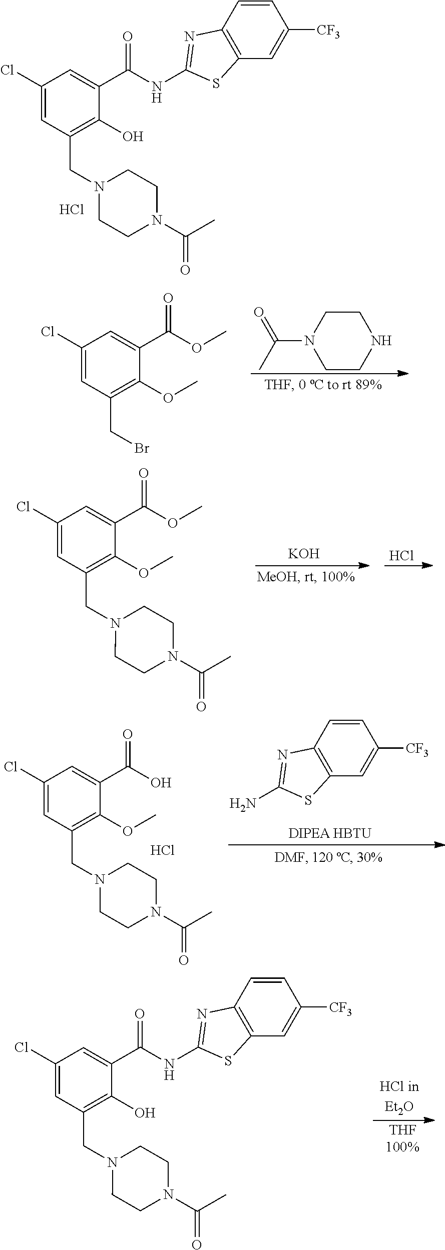

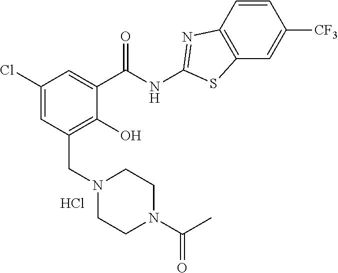 [SMILES: CC(=O)N1CCN(Cc2cc(Cl)cc(C(=O)Nc3nc4ccc(C(F)(F)F)cc4s3)c2O)CC1 ; Cl]

At 0° C., to a stirred solution of methyl 3-(bromomethyl)-5-chloro-2-methoxybenzoate (257 mg, 0.88 mmol, Example 2) in THF (5 mL) was added 1-(piperazin-1-yl)ethan-1-one (225 mg, 1.754 mmol) and the mixture was stirred at rt for 6 h. After completion of the reaction, the reaction was partitioned between DCM and water. The organic layer was dried over sodium sulfate and concentrated under reduced pressure. The residue was purified by flash column to afford methyl 3-((4-acetylpiperazin-1-yl)methyl)-5-chloro-2-methoxybenzoate (265 mg, 89%) as a colorless oil. ¹H NMR (300 MHz, cdcl₃) δ 7.62 (d, J=2.8 Hz, 1H), 7.51 (d, J=2.8 Hz, 1H), 3.84 (s, 3H), 3.75 (s, 3H), 3.57-3.52 (m, 2H), 3.48 (s, 2H), 3.42-3.37 (m, 2H), 2.43-2.36 (m, 4H), 2.00 (s, 3H).

To a stirred solution of methyl 3-((4-acetylpiperazin-1-yl)methyl)-5-chloro-2-methoxybenzoate (265 mg, 0.78 mmol) in MeOH (5 mL) was added 4.0 mL 1N KOH solution. The resulting mixture was stirred at rt overnight. The solvent was evaporated out and 4N HCl in dioxane (2 mL) was added to the residue. The mixture was stirred for another 10 minutes before it was concentrated and dried under vacumn. To this residue was added HBTU (356 mg, 0.94 mmol), DMF (5 mL) and DIPEA (680 μL, 3.9 mmol). The mixture was stirred for 10 minutes and then 6-(trifluoromethyl)benzo[d]thiazol-2-amine (170 mg, 0.78 mmol) was added. The resulting reaction was heated at 120° C. for 24 h. After cooling to rt, the mixture was separated between ethyl acetate and water. The organic layer was washed with brine, dried over sodium sulfate and concentrated in vacuo. Purification by column chromatography gave 3-((4-acetylpiperazin-1-yl)methyl)-5-chloro-2-hydroxy-N-(6-(trifluoromethyl)benzo[d]thiazol-2-yl)benzamide (101 mg, 30%) as a yellow solid. ¹H NMR (300 MHz, cdcl₃) δ 8.19 (d, J=2.6 Hz, 1H), 8.15 (s, 1H), 7.90 (d, J=8.6 Hz, 1H), 7.70 (d, J=8.5 Hz, 1H), 7.26-7.20 (m, 1H), 3.57-3.52 (m, 2H), 3.48 (s, 2H), 3.42-3.37 (m, 2H), 2.43-2.36 (m, 4H), 2.00 (s, 3H). MS (ESI) [M+H]⁺ requires m/z 513.10, found m/z 513.30.

To a stirred solution of 3-((4-acetylpiperazin-1-yl)methyl)-5-chloro-2-hydroxy-N-(6-(trifluoromethyl)benzo[d]thiazol-2-yl)benzamide (101 mg, 0.197 mmol) in THF (5 mL) was added 2N HCl in ether (100 μL, 0.2 mmol). The mixture was stirred at rt for 20 minutes. The resulting precipitate was filtered and washed with diethyl ether to afford 3-((4-acetylpiperazin-1-yl)methyl)-5-chloro-2-hydroxy-N-(6-(trifluoromethyl)benzo[d]thiazol-2-yl)benzamide hydrochloride as a yellow solid (100%). MS (ESI) [M+H]⁺ requires m/z 513.10, found m/z 513.30.

### Example 20

### 5-chloro-2-hydroxy-3-((2-methoxyethoxy)methyl)-N-(6-(trifluoromethyl)pyridin-3-yl)benzamide (20) and 5-chloro-2-hydroxy-3-((2-methoxyethoxy)methyl)-N-(6-(trifluoromethyl)pyridin-3-yl)benzamide hydrochloride (20A)

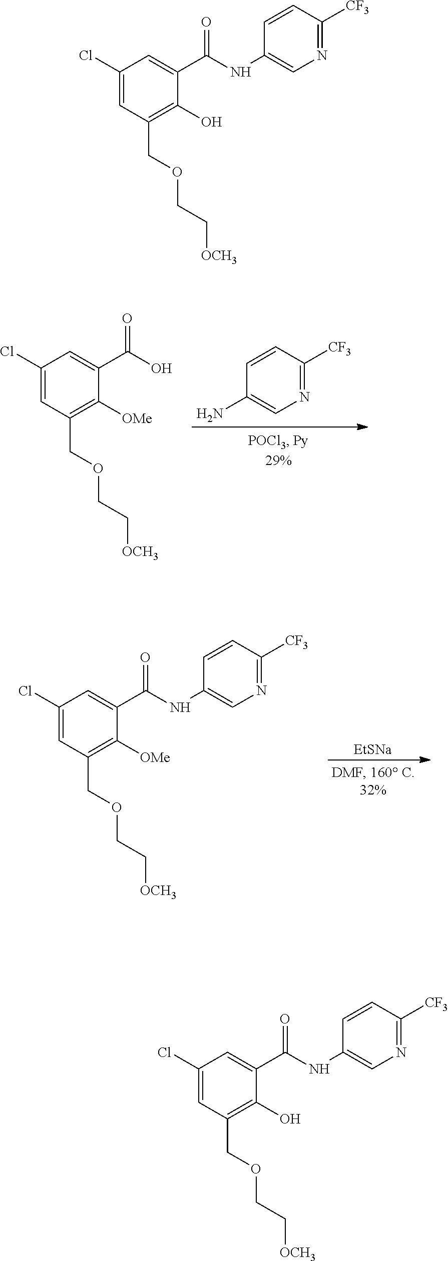 [SMILES: COCCOCc1cc(Cl)cc(C(=O)Nc2ccc(C(F)(F)F)nc2)c1O ; "EtSNa" ; COCCOCc1cc(Cl)cc(C(=O)O)c1OC ; Nc1ccc(C(F)(F)F)nc1 ; COCCOCc1cc(Cl)cc(C(=O)Nc2ccc(C(F)(F)F)nc2)c1OC ; COCCOCc1cc(Cl)cc(C(=O)Nc2ccc(C(F)(F)F)nc2)c1O]

To a solution of 5-chloro-2-methoxy-3-((2-methoxyethoxy)methyl)benzoic acid (200 mg, 728.08 μmol, Example 2), 6-(trifluoromethyl)pyridin-3-amine (118 mg, 728.08 μmol) in pyridine (2 mL) was added POCl₃ (167 mg, 1.09 mmol, 101.49 μL) at 0° C. The mixture was stirred at 0° C. for 1 h. The reaction mixture was concentrated under reduced pressure to remove solvent. The residue was diluted with water (3 mL) and extracted with DCM (3 mL×3). The combined organic layers were washed with brine 5 mL, dried over Na₂SO₄, filtered and concentrated under reduced pressure to give a residue. The residue was purified by prep-TLC (SiO₂, petroleum ether/ethyl acetate=3:1) to afford 5-chloro-2-methoxy-3-((2-methoxyethoxy)methyl)-N-(6-(trifluoromethyl)pyridine-3-ylbenzamide (117 mg, 212.32 μmol, 29.16% yield, 76% purity) as a white solid. MS (ESI) [M+H]⁺ requires m/z 419.09, found m/z 419.2

To 5-chloro-2-methoxy-3-((2-methoxyethoxy)methyl)-N-(6-(trifluoromethyl)pyridine-3-ylbenzamide (95 mg, 226.84 μmol) in DMF (2 mL) was added NaSEt (38 mg, 453.68 μmol) and the mixture was stirred at 160° C. for 2 h. The reaction mixture was concentrated under reduced pressure to remove solvent. The residue was purified by prep-HPLC (HCl condition) to afford 5-chloro-2-hydroxy-3-((2-methoxyethoxy)methyl)-N-(6-(trifluoromethyl)pyridin-3-yl)benzamide (32.99 mg, 73.67 μmol, 32.48% yield, 98.537% purity, HCl) as a brown solid. MS (ESI) [M+H]⁺ requires m/z 405.08, found m/z 405.0. ¹H NMR (400 MHz, METHANOL-d₄) δ ppm 8.93 (d, J=2.19 Hz, 1H), 8.37 (dd, J=8.33, 2.19 Hz, 1H), 7.90 (d, J=2.19 Hz, 1H), 7.74 (d, J=8.77 Hz, 1H), 7.52 (d, J=2.63 Hz, 1H), 4.53 (s, 2H), 3.60-3.66 (m, 2H), 3.51-3.56 (m, 2H), 3.22 (dt, J=3.29, 1.43 Hz, 3H).

### Example 21

### 5-chloro-2-hydroxy-3-((2-methoxyethoxy)methyl)-N-(2-(trifluoromethyl)pyrimidin-5-yl)benzamide (21)

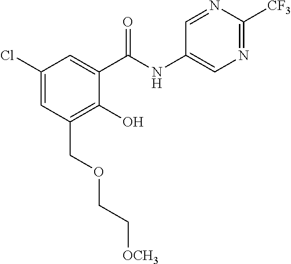 [SMILES: COCCOCc1cc(Cl)cc(C(=O)Nc2cnc(C(F)(F)F)nc2)c1O]

Using a method similar to Example 20, 5-chloro-2-hydroxy-3-((2-methoxyethoxy)methyl)-N-(2-(trifluoromethyl)pyrimidin-5-yl)benzamide was obtained. MS (ESI) [M+H]⁺ requires m/z 406.07, found m/z 406.0. ¹H NMR (400 MHz, METHANOL-d₄) δ ppm 9.35 (s, 2H), 8.00 (d, J=2.43 Hz, 1H), 7.64 (s, 1H), 4.64 (s, 2H) 3.73 (dd, J=5.62, 3.42 Hz, 2H), 3.60-3.66 (m, 2H), 3.40 (s, 3H).

### Example 22

### 5-chloro-2-hydroxy-3-(((2-methoxyethyl)amino)methyl)-N-(6-(trifluoromethyl)benzo[d]thiazol-2-yl)benzamide (22)

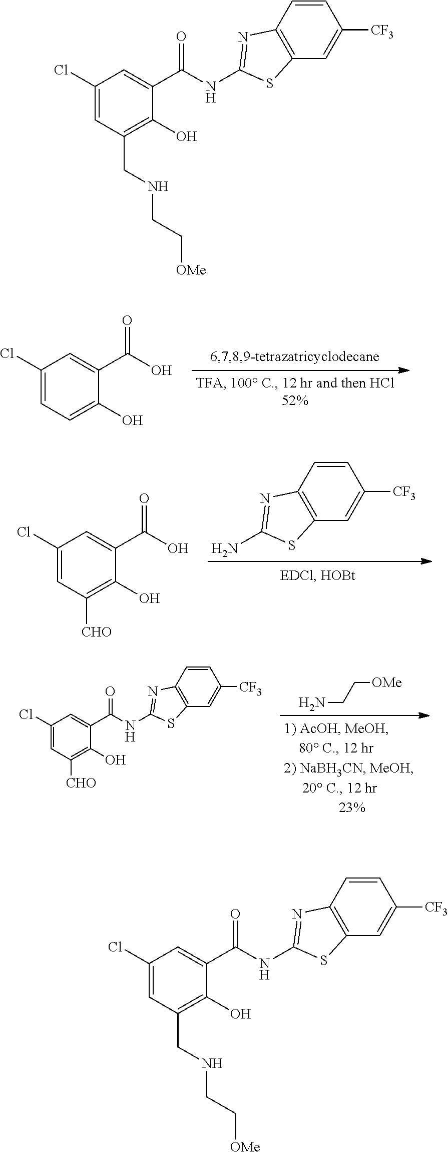

A solution of 5-chloro-2-hydroxybenzoic acid (10 g, 57.95 mmol), and 6,7,8,9-tetrazatricyclodecane (16.2 g, 115.90 mmol) in TFA (100 mL) was stirred at 100° C. for 12 h, then cooled to 20° C., followed by addition of HCl (100 mL, 3M). The reaction mixture was stirred for another 1 h, then concentrated under reduced pressure. The white residue was suspend in water (50 mL). The solid was collected and dried under vacuum to obtained 5-chloro-3-formyl-2-hydroxybenzoic acid (6 g, crude) as a white solid. ¹H NMR (400 MHz, DMSO-d₆) δ ppm 10.30 (s, 1H), 8.02 (br d, J=2.45 Hz, 1H), 7.83 (br d, J=2.93 Hz, 1H)

A mixture of 5-chloro-3-formyl-2-hydroxybenzoic acid (2 g, 9.97 mmol), 6-(trifluoromethyl)benzo[d]thiazol-2-amine (2.1 g, 9.97 mmol) and EDCI (2.2 g, 11.97 mmol), HOBt (2.0 g, 14.96 mmol) in DCM (20 mL) was heated at 50° C. for 8 h. The reaction mixture was concentrated to remove solvent, then H₂O (20 mL) was added and NaOH (3M) was added until the pH was ~9-10. The reaction mixture was extracted with ethyl acetate (30 mL×2), then concentrated to remove ethyl acetate. The residue was diluted with ethyl acetate (30 mL), solid precipitated, filtered and concentrated under reduced pressure to give 5-chloro-3-formyl-2-hydroxy-N-(6-(trifluoromethyl)benzo[d]thiazol-2-yl)benzamide (580 mg, 5.8% yield) as a yellow solid. ¹H NMR (400 MHz, DMSO-d₆) δ ppm 10.34 (s, 1H), 8.43 (s, 1H), 7.95 (d, J=3.09 Hz, 1H), 7.86 (d, J=8.38 Hz, 1H), 7.70 (br d, J=8.60 Hz, 1H), 7.49 (d, J=3.31 Hz, 1H). MS (ESI) [M+H]⁺ requires m/z 400.99, found m/z 401.1.

To a solution of 2-methoxyethanamine (22 mg, 299.43 μmol), 5-chloro-3-formyl-2-hydroxy-N-(6-(trifluoromethyl)benzo[d]thiazol-2-yl)benzamide (100 mg, 249.53 μmol) in MeOH (4 mL) was added acetic acid (3 mg, 49.91 μmol, 2.85 μL) to make pH ~5-6. The mixture was stirred at 20° C. for 2 h, then NaBH₃CN (94 mg, 1.50 mmol) was added. The mixture was stirred at 20° C. for another 10 h. The reaction mixture was concentrated to remove solvent. The residue was purified by prep-HPLC (HCl condition) to afford 5-chloro-2-hydroxy-3-(((2-methoxyethyl)amino)methyl)-N-(6-(trifluoromethyl)benzo[d]thiazol-2-yl)benzamide (28.30 mg, 57.02 μmol, 22.85% yield, 100% purity, HCl) as a brown solid. ¹H NMR (400 MHz, METHANOL-d₄) δ ppm 8.27 (s, 1H), 8.20 (d, J=2.45 Hz, 1H), 7.74-7.82 (m, 2H), 7.63 (d, J=2.45 Hz, 1H), 4.31 (s, 2H), 3.67-3.72 (m, 2H), 3.44 (s, 3H), 3.28 (d, J=4.89 Hz, 2H). MS (ESI) [M+H]⁺ requires m/z 460.06, found m/z 460.2

### Example 23

### 5-chloro-2-hydroxy-3-(pyrrolidin-1-ylmethyl)-N-(6-(trifluoromethyl)benzo[d]thiazol-2-yl)benzamide (23)

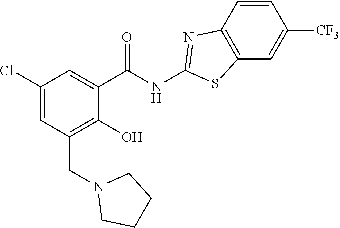 [SMILES: O=C(Nc1nc2ccc(C(F)(F)F)cc2s1)c1cc(Cl)cc(CN2CCCC2)c1O]

5-chloro-2-hydroxy-3-(pyrrolidin-1-ylmethyl)-N-(6-(trifluoromethyl)benzo[d]thiazol-2-yl)benzamide was made in a similar manner as the described in Example 22. MS (ESI) [M+H]⁺ requires m/z 456.07, found m/z 456.2. ¹H NMR (400 MHz, METHANOL-d4) δ ppm 8.16-8.32 (m, 2H), 7.73-7.83 (m, 2H), 7.70 (s, 1H), 4.46 (s, 2H), 3.51-3.63 (m, 2H), 3.25-3.30 (m, 2H), 2.15-2.28 (m, 2H), 1.99-2.11 (m, 2H)

### Example 24

### 5-chloro-2-hydroxy-3-(((2-(2-methoxyethoxy)ethyl)(methyl)amino)methyl)-N-(6-(trifluoromethyl)benzo[d]thiazol-2-yl)benzamide (24) and 5-chloro-2-hydroxy-3-(((2-(2-methoxyethoxy)ethyl)(methyl)amino)methyl)-N-(6-(trifluoromethyl)benzo[d]thiazol-2-yl)benzamide hydrochloride (24A)

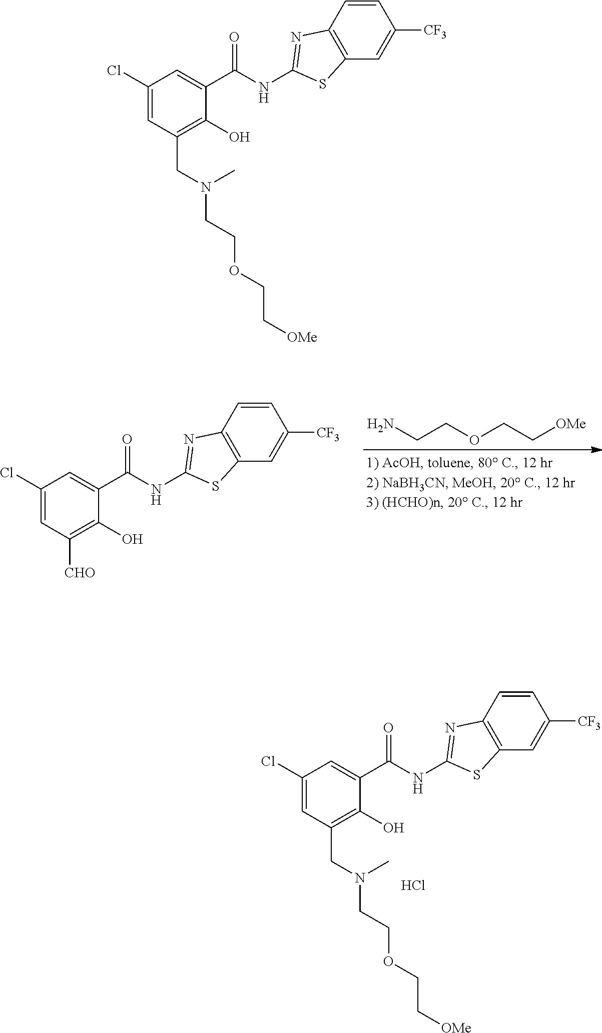 [SMILES: COCCOCCN(C)Cc1cc(Cl)cc(C(=O)Nc2nc3ccc(C(F)(F)F)cc3s2)c1O ; O=Cc1cc(Cl)cc(C(=O)Nc2nc3ccc(C(F)(F)F)cc3s2)c1O ; COCCOCCN ; COCCOCCN(C)Cc1cc(Cl)cc(C(=O)Nc2nc3ccc(C(F)(F)F)cc3s2)c1O ; Cl]

To a solution of 2-(2-methoxyethoxy)ethanamine (53 mg, 449.15 μmol, 44.21 μL), 5-chloro-3-formyl-2-hydroxy-N-(6-(trifluoromethyl)benzo[d]thiazol-2-yl)benzamide (150 mg, 374.29 μmol) in toluene (3 mL) was added acetic acid (22 mg, 374.29 μmol, 21.41 μL) to pH ~5-6. The mixture was stirred at 80° C. for 12 h. The reaction mixture was concentrated to remove solvent then MeOH (3 mL), and NaBH₃CN (141 mg, 2.25 mmol) were added. The mixture was stirred at 20° C. for another 12 h, then (HCHO)n (101 mg, 1.12 mmol) was added and the mixture was stirred at 20° C. for another 12 h. The reaction mixture was concentrated to remove solvent. then purified by prep-HPLC (HCl condition) to afford 5-chloro-2-hydroxy-3-(((2-(2-methoxyethoxy)ethyl)(methyl)amino)methyl)-N-(6-(trifluoromethyl)benzo[d]thiazol-2-yl)benzamide (55.74 mg, 100.01 μmol, 26.72% yield, 99.478% purity, HCl) as a white solid. MS (ESI) [M+H]⁺ requires m/z 518.1, found m/z 518.2. ¹H NMR (400 MHz, METHANOL-d₄) δ ppm 8.20-8.30 (m, 2H), 7.74-7.83 (m, 2H), 7.71 (br d, J=2.21 Hz, 1H), 4.62 (br d, J=11.25 Hz, 1H), 4.35 (br d, J=12.57 Hz, 1H), 3.90 (br d, J=4.41 Hz, 2H), 3.67-3.75 (m, 2H), 3.58-3.65 (m, 2H), 3.51 (br s, 1H), 3.44 (br s, 1H), 3.41 (s, 3H), 2.92 (s, 3H).

### Example 25

### 3-((bis(2-methoxyethyl)amino)methyl)-5-chloro-2-hydroxy-N-(6-(trifluoromethyl)benzo[d]thiazol-2-yl)benzamide (25) and 3-((bis(2-methoxyethyl)amino)methyl)-5-chloro-2-hydroxy-N-(6-(trifluoromethyl)benzo[d]thiazol-2-yl)benzamide hydrochloride (25A)

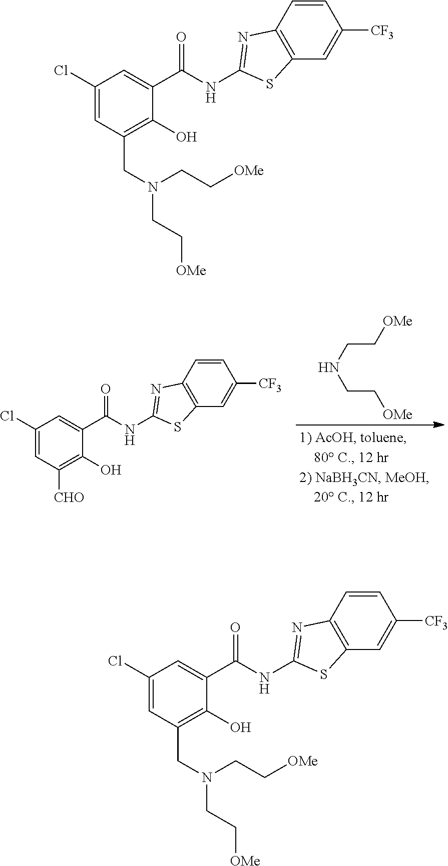 [SMILES: COCCN(CCOC)Cc1cc(Cl)cc(C(=O)Nc2nc3ccc(C(F)(F)F)cc3s2)c1O ; O=Cc1cc(Cl)cc(C(=O)Nc2nc3ccc(C(F)(F)F)cc3s2)c1O ; COCCNCCOC ; COCCN(CCOC)Cc1cc(Cl)cc(C(=O)Nc2nc3ccc(C(F)(F)F)cc3s2)c1O]

To a solution of bis(2-methoxyethyl)amine (40 mg, 299.43 μmol, 44.21 μL) and 5-chloro-3-formyl-2-hydroxy-N-(6-(trifluoromethyl)benzo[d]thiazol-2-yl)benzamide (100 mg, 249.53 μmol, Example 22) in toluene (3 mL) was added acetic acid (3 mg, 49.91 μmol, 2.85 μL) to pH ~5-6. The mixture was stirred at 75° C. for 12 h. The reaction mixture was concentrated to remove solvent, then MeOH (3 mL) and NaBH₃CN (94 mg, 1.5 mmol) were added. The mixture was stirred at 20° C. for another 5 h. The reaction mixture was concentrated to remove solvent. The residue was purified by prep-HPLC (HCl condition) to afford 3-((bis(2-methoxyethyl)amino)methyl)-5-chloro-2-hydroxy-N-(6-(trifluoromethyl)benzo[d]thiazol-2-yl)benzamide (98.93% purity, HCl) 47.28 mg as a brown solid. MS (ESI) [M+H]⁺ requires m/z 518.1, found m/z 518.1. ¹H NMR (400 MHz, METHANOL-d4) δ ppm 8.26 (s, 1H), 8.15 (d, J=2.44 Hz, 1H), 7.73-7.80 (m, 2H), 7.56 (d, J=2.45 Hz, 1H), 4.52 (s, 2H), 3.74-3.80 (m, 4H), 3.44-3.50 (m, 4H), 3.41 (s, 6H).

Compounds 26-37 were made in a similar manner as Compound 25 in Example 25:

### Example 26

### 5-chloro-2-hydroxy-3-(piperidin-1-ylmethyl)-N-(6-(trifluoromethyl)benzo[d]thiazol-2-yl)benzamide (26)

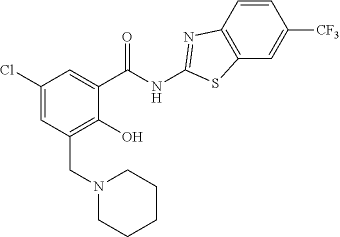 [SMILES: O=C(Nc1nc2ccc(C(F)(F)F)cc2s1)c1cc(Cl)cc(CN2CCCCC2)c1O]

MS (ESI) [M+H]⁺ requires m/z 470.08, found m/z 470.0. ¹H NMR (400 MHz, METHANOL-d4) δ ppm 8.24-8.29 (m, 2H), 7.74-7.82 (m, 2H), 7.68 (d, J=2.45 Hz, 1H), 4.36 (s, 2H), 3.46-3.59 (m, 2H), 3.02-3.18 (m, 2H), 1.96 (br d, J=14.18 Hz, 2H), 1.77 (br d, J=13.69 Hz, 3H), 1.56 (br s, 1H).

### Example 27

### 3-(azetidin-1-ylmethyl)-5-chloro-2-hydroxy-N-(6-(trifluoromethyl)benzo[d]thiazol-2-yl)benzamide (27)

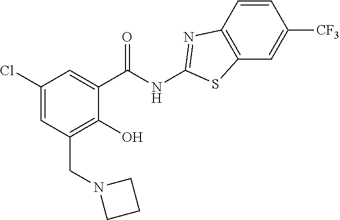 [SMILES: O=C(Nc1nc2ccc(C(F)(F)F)cc2s1)c1cc(Cl)cc(CN2CCC2)c1O]

MS (ESI) [M+H]⁺ requires m/z 442.85, found m/z 442.1. ¹H NMR (400 MHz, METHANOL-d4) δ ppm 8.28 (s, 1H) 8.24 (d, J=2.65 Hz, 1H) 7.74-7.84 (m, 2H) 7.65 (d, J=2.21 Hz, 1H)

### Example 28

### 5-chloro-2-hydroxy-3-((4-methylpiperazin-1-yl)methyl)-N-(6-(trifluoromethyl)benzo[d]thiazol-2-yl)benzamide (28)

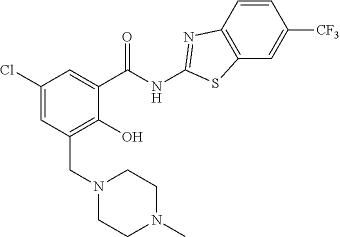 [SMILES: CN1CCN(Cc2cc(Cl)cc(C(=O)Nc3nc4ccc(C(F)(F)F)cc4s3)c2O)CC1]

MS (ESI) [M+H]⁺ requires m/z 485.09, found m/z 485.0. ¹H NMR (400 MHz, METHANOL-d4) δ ppm 8.27 (s, 1H), 8.25 (d, J=2.93 Hz, 1H), 7.74-7.82 (m, 3H), 4.50 (s, 2H), 3.43-3.88 (m, 8H), 3.02 (s, 3H)

### Example 29

### 5-chloro-2-hydroxy-3-((3-oxopiperazin-1-yl)methyl)-N-(6-(trifluoromethyl)benzo[d]thiazol-2-yl)benzamide (29)

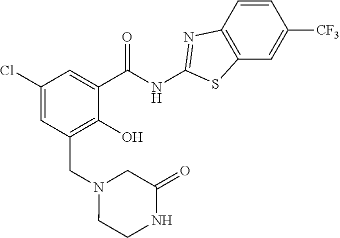 [SMILES: O=C1CN(Cc2cc(Cl)cc(C(=O)Nc3nc4ccc(C(F)(F)F)cc4s3)c2O)CCN1]

MS (ESI) [M+H]⁺ requires m/z 485.06, found m/z 485.0. ¹H NMR (400 MHz, METHANOL-d4) δ ppm 8.28-8.31 (m, 2H), 7.76-7.84 (m, 2H), 7.72 (d, J=2.63

### Hz, 1H), 4.53 (s, 2H), 3.95 (s, 2H), 3.60 (br s, 4H)

### Example 30

### 5-chloro-2-hydroxy-3-((3-hydroxypyrrolidin-1-yl)methyl)-N-(6-(trifluoromethyl)benzo[d]thiazol-2-yl)benzamide (30)

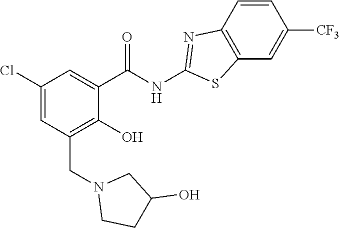 [SMILES: O=C(Nc1nc2ccc(C(F)(F)F)cc2s1)c1cc(Cl)cc(CN2CCC(O)C2)c1O]

MS (ESI) [M+H]⁺ requires m/z 472.06 found m/z 472.0. ¹H NMR (400 MHz, DMSO-d6) δ ppm 8.41 (s, 1H), 8.05 (d, J=2.87 Hz, 1H), 7.70-7.85 (m, 3H), 4.43 (br d, J=2.20 Hz, 1H), 4.39 (s, 2H), 3.41-3.50 (m, 2H), 3.32-3.40 (m, 1H), 3.15.

### Example 31

### 5-chloro-2-hydroxy-3-((3-hydroxy-3-methylpyrrolidin-1-yl)methyl)-N-(6-(trifluoromethyl)benzo[d]thiazol-2-yl)benzamide (31)

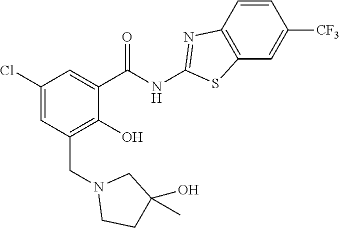 [SMILES: CC1(O)CCN(Cc2cc(Cl)cc(C(=O)Nc3nc4ccc(C(F)(F)F)cc4s3)c2O)C1]

MS (ESI) [M+H]⁺ requires m/z 486.08 found m/z 486.0. ¹H NMR (400 MHz, METHANOL-d4) δ ppm 8.23 (br d, J=16.63 Hz, 2H), 7.77 (q, J=8.64 Hz, 2H), 7.69 (s, 1H), 4.41-4.59 (m, 2H), 3.47-3.87 (m, 2H), 3.34-3.45 (m, 1.5H), 3.22 (d, J=11.74 Hz, 0.5H), 1.97-2.30 (m, 2H), 1.46 (s, 3H).

### Example 32

### 5-chloro-2-hydroxy-3-((3-methoxypyrrolidin-1-yl)methyl)-N-(6-(trifluoromethyl)benzo[d]thiazol-2-yl)benzamide (32)

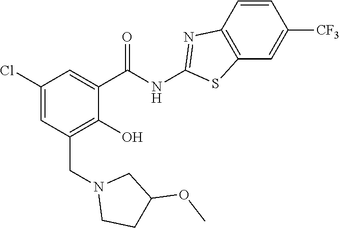 [SMILES: COC1CCN(Cc2cc(Cl)cc(C(=O)Nc3nc4ccc(C(F)(F)F)cc4s3)c2O)C1]

MS (ESI) [M+H]⁺ requires m/z 486.08 found m/z 486.1. ¹H NMR (400 MHz, METHANOL-d4) δ ppm 8.19-8.28 (m, 2H), 7.77 (q, J=8.53 Hz, 2H), 7.69 (br s, 1H), 4.43-4.55 (m, 2H), 4.16-4.24 (m, 1H), 3.56-3.74 (m, 2H), 3.32-3.50 (m, 5H), 2.30-2.48 (m, 1H), 2.06-2.21 (m, 1H).

### Example 33

### 3-((3-acetamidopyrrolidin-1-yl)methyl)-5-chloro-2-hydroxy-N-(6-(trifluoromethyl)benzo[d]thiazol-2-yl)benzamide (33)

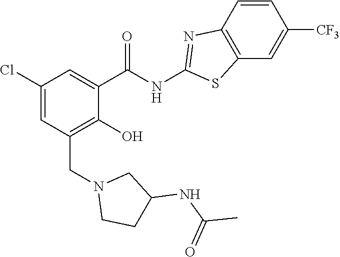 [SMILES: CC(=O)NC1CCN(Cc2cc(Cl)cc(C(=O)Nc3nc4ccc(C(F)(F)F)cc4s3)c2O)C1]

MS (ESI) [M+H]⁺ requires m/z 513.09 found m/z 513.0. 1H NMR (400 MHz, DMSO-d6) δ ppm 8.45 (s, 1H) 8.24 (br d, J=5.07 Hz, 1H) 8.11 (d, J=2.65 Hz, 1H) 7.84-7.91 (m, 2H) 7.77-7.83 (m, 1H) 4.44 (s, 2H) 4.40 (br d, J=6.39 Hz, 1H) 3.47-3.63 (m, 2H) 3.32-3.44 (m, 1H) 3.18-3.27 (m, 1H) 2.27-2.39 (m, 1H) 1.89-2.01 (m, 1H) 1.84 (s, 3H)

### Example 34

### 1-(5-chloro-2-hydroxy-3-((6-(trifluoromethyl)benzo[d]thiazol-2-yl)carbamoyl)benzyl)pyrrolidine-2-carboxamide (34)

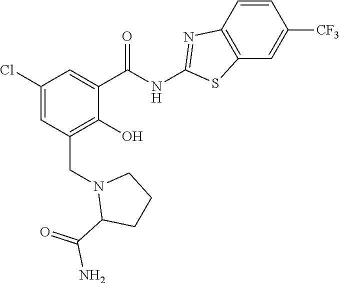 [SMILES: NC(=O)C1CCCN1Cc1cc(Cl)cc(C(=O)Nc2nc3ccc(C(F)(F)F)cc3s2)c1O]

MS (ESI) [M+H]⁺ requires m/z 499.07 found m/z 498.9. ¹H NMR (400 MHz, METHANOL-d4) δ ppm 8.28 (s, 1H), 8.23 (d, J=2.65 Hz, 1H), 7.74-7.84 (m, 2H), 7.67 (d, J=2.43 Hz, 1H), 4.45-4.58 (m, 2H), 4.33 (dd, J=9.37, 6.73 Hz, 1H), 3.73 (br s, 1H), 3.42-3.50 (m, 2H), 2.51-2.67 (m, 1H), 1.99-2.28 (m, 2H)

### Example (35)

### 5-chloro-3-((1,1-dioxidothiomorpholino)methyl)-2-hydroxy-N-(6-(trifluoromethyl)benzo[d]thiazol-2-yl)benzamide (35)

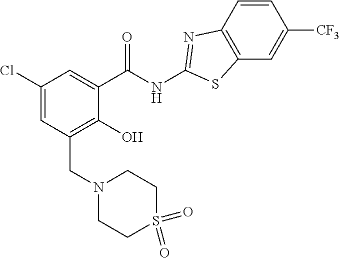 [SMILES: O=C(Nc1nc2ccc(C(F)(F)F)cc2s1)c1cc(Cl)cc(CN2CCS(=O)(=O)CC2)c1O]

MS (ESI) [M+H]⁺ requires m/z 520.03 found m/z 520.0. ¹H NMR (400 MHz, METHANOL-d4) δ ppm 8.27-8.31 (m, 2H), 7.73-7.84 (m, 3H), 4.59 (s, 2H), 3.91 (br d, J=5.73 Hz, 4H), 3.57 (br s, 4H)

### Example 36

### 5-chloro-3-((3-cyanopyrrolidin-1-yl)methyl)-2-hydroxy-N-(6-(trifluoromethyl)benzo[d]thiazol-2-yl)benzamide (36)

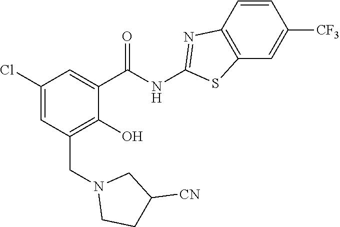 [SMILES: N#CC1CCN(Cc2cc(Cl)cc(C(=O)Nc3nc4ccc(C(F)(F)F)cc4s3)c2O)C1]

MS (ESI) [M+H]⁺ requires m/z 481.06 found m/z 480.9. ¹H NMR (400 MHz, DMSO-d6) δ ppm 8.42 (s, 1H) 7.78-7.86 (m, 2H) 7.69 (br d, J=8.38 Hz, 1H) 7.38 (d, J=2.87 Hz, 1H) 4.25 (br s, 2H) 3.62 (br s, 1H) 3.49-3.56 (m, 4H) 2.16-2.36 (m, 2H)

### Example 37

### 5-chloro-2-hydroxy-3-((3-(methylsulfonyl)pyrrolidin-1-yl)methyl)-N-(6-(trifluoromethyl)benzo[d]thiazol-2-yl)benzamide (37)

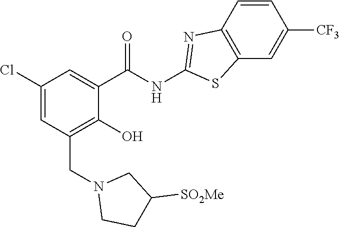 [SMILES: CS(=O)(=O)C1CCN(Cc2cc(Cl)cc(C(=O)Nc3nc4ccc(C(F)(F)F)cc4s3)c2O)C1]

MS (ESI) [M+H]⁺ requires m/z 534.05 found m/z 534.0. 1H NMR (400 MHz, METHANOL-d4) δ ppm 8.22-8.30 (m, 2H), 7.74-7.84 (m, 2H), 7.70 (br d, J=2.45 Hz, 1H), 4.51-4.58 (m, 2H), 4.16-4.27 (m, 1H), 3.83-3.99 (m, 2H), 3.60 (br s, 2H), 3.10 (s, 3H), 2.50-2.69 (m, 2H)

### Example 38

### (E)-5-chloro-2-hydroxy-3-((hydroxyimino)methyl)-N-(6-(trifluoromethyl)benzo[d]thiazol-2-yl)benzamide (38)

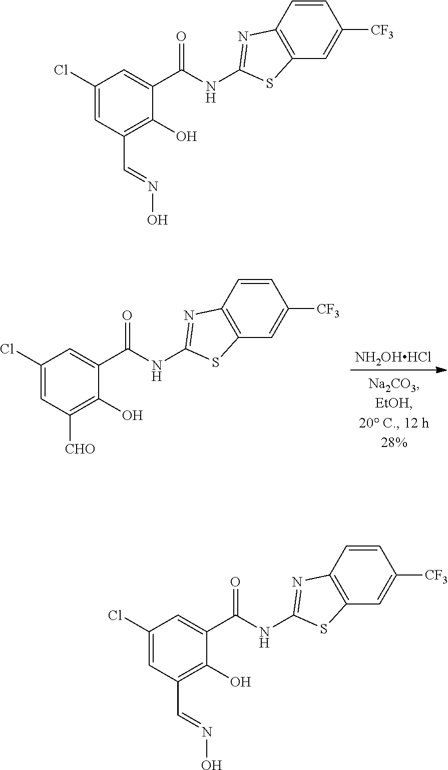 [SMILES: O=C(Nc1nc2ccc(C(F)(F)F)cc2s1)c1cc(Cl)cc(/C=N/O)c1O ; O=Cc1cc(Cl)cc(C(=O)Nc2nc3ccc(C(F)(F)F)cc3s2)c1O ; "NH2OH·HCl" ; O=C(Nc1nc2ccc(C(F)(F)F)cc2s1)c1cc(Cl)cc(/C=N/O)c1O]

To a solution of 5-chloro-3-formyl-2-hydroxy-N-(6-(trifluoromethyl)benzo[d]thiazol-2-yl)benzamide (100 mg, 249.53 gmol) in EtOH (3 mL) was added NH₂OH·HCl (17.34 mg, 249.53 μmol) and Na₂CO₃ (52.89 mg, 499.05 μmol). The mixture was stirred at 20° C. for 12 h. The reaction mixture was concentrated under reduced pressure to remove solvent. The residue was purified by prep-HPLC (basic condition) to afford the title compound (E)-5-chloro-2-hydroxy-3-((hydroxyimino)methyl)-N-(6-(trifluoromethyl)benzo[d]thiazol-2-yl)benzamide (30.95 mg, 70.19 gmol, 28.13% yield, 94.297% purity) as a yellow solid. ¹H NMR (400 MHz, METHANOL-d₄) δ ppm 8.38 (br s, 1H), 8.30 (br s, 1H), 8.05 (br s, 1H), 7.87 (br s, 1H), 7.72 (br d, J=16.63 Hz, 2H). MS (ESI) [M+H]⁺ requires m/z 416.00, found m/z 416.00.

### Example 39

### (E)-5-chloro-2-hydroxy-3-((methoxyimino)methyl)-N-(6-(trifluoromethyl)benzo[d]thiazol-2-yl)benzamide (39)

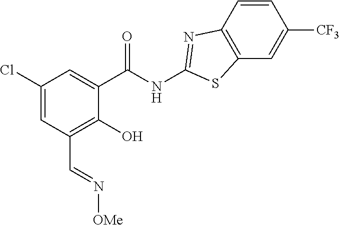 [SMILES: CO/N=C/c1cc(Cl)cc(C(=O)Nc2nc3ccc(C(F)(F)F)cc3s2)c1O]

(E)-5-chloro-2-hydroxy-3-((methoxyimino)methyl)-N-(6-(trifluoromethyl)benzo[d]thiazol-2-yl)benzamide was made in a similar manner as compound 38. MS (ESI) [M+H]⁺ requires 430.02 m/z found m/z 430.0. ¹H NMR (400 MHz, DMSO-d₆) δ ppm 8.52 (br s, 1H), 8.41 (br s, 1H), 8.01 (br s, 1H), 7.83 (br s, 2H), 7.76 (br d, J=0.98 Hz, 1H), 3.94 (br s, 3H).

### Example 40

### 3-(acetamidomethyl)-5-chloro-2-hydroxy-N-(6-(trifluoromethyl)benzo[d]thiazol-2-yl)benzamide (40)

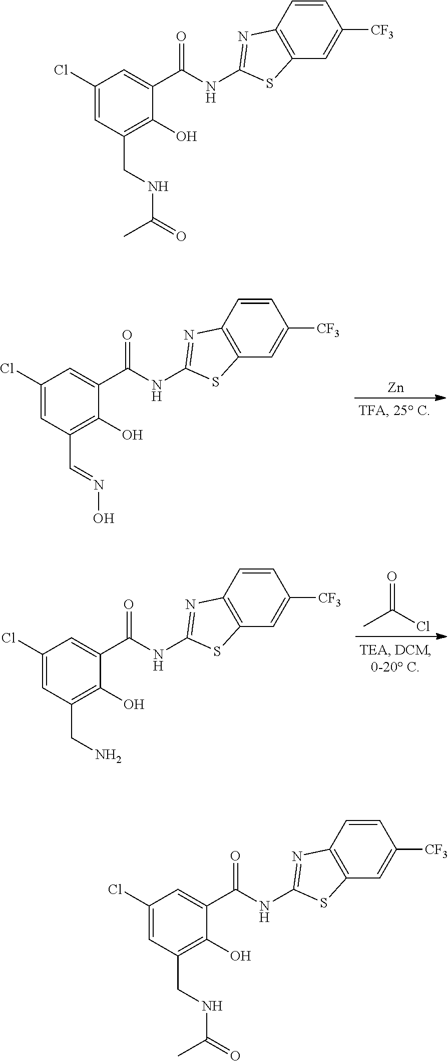

To a solution of (E)-5-chloro-2-hydroxy-3-((hydroxyimino)methyl)-N-(6-(trifluoromethyl)benzo[d]thiazol-2-yl)benzamide (320 mg, 769.65 μmol, Example 40) in TFA (6 mL) was added Zn (251 mg, 3.85 mmol). The mixture was stirred at 25° C. for 12 h. The reaction mixture was concentrated to remove solvent. The residue was diluted with water (3 mL) and adjusted to pH 9 with 2M NaOH, then extracted with ethyl acetate (5 mL×3). The combined organic layer was washed with brine (15 mL), dried over Na₂SO₄, filtered and concentrated under reduced pressure to afford 3-(aminomethyl)-5-chloro-2-hydroxy-N-(6-(trifluoromethyl)benzo[d]thiazol-2-yl)benzamide (300 mg, crude) as a white solid.

3-(aminomethyl)-5-chloro-2-hydroxy-N-(6-(trifluoromethyl)benzo[d]thiazol-2-yl)benzamide (20 mg, 49.78 μmol) and TEA (6 mg, 59.73 μmol, 8.31 μL) was dissolved in DCM (3 mL) at 0° C. The solution was stirred and acetyl chloride (3 mg, 39.82 μmol, 2.84 μL) was added. The resulting solution was warmed to 20° C. for 30 minutes. The reaction mixture was quenched by addition of MeOH (3 mL), then concentrated under reduced pressure to give a residue. The residue was purified by prep-HPLC (HCl condition) to afford 3-(acetamidomethyl)-5-chloro-2-hydroxy-N-(6-(trifluoromethyl)benzo[d]thiazol-2-yl)benzamide (14 mg, 99.660% purity) as a white solid. ¹H NMR (400 MHz, METHANOL-d₄) δ ppm 8.28 (s, 1H), 8.03 (d, J=2.45 Hz, 1H), 7.73-7.86 (m, 2H), 7.43 (s, 1H), 4.39 (s, 2H), 2.03 (s, 3H) MS (ESI) [M+H]⁺ requires m/z 444.03, found m/z 444.0.

### Example 41

### 5-chloro-2-hydroxy-N¹,N¹-bis(2-methoxyethyl)-N³-(6-(trifluoromethyl)benzo[d]thiazol-2-yl)isophthalamide (41)

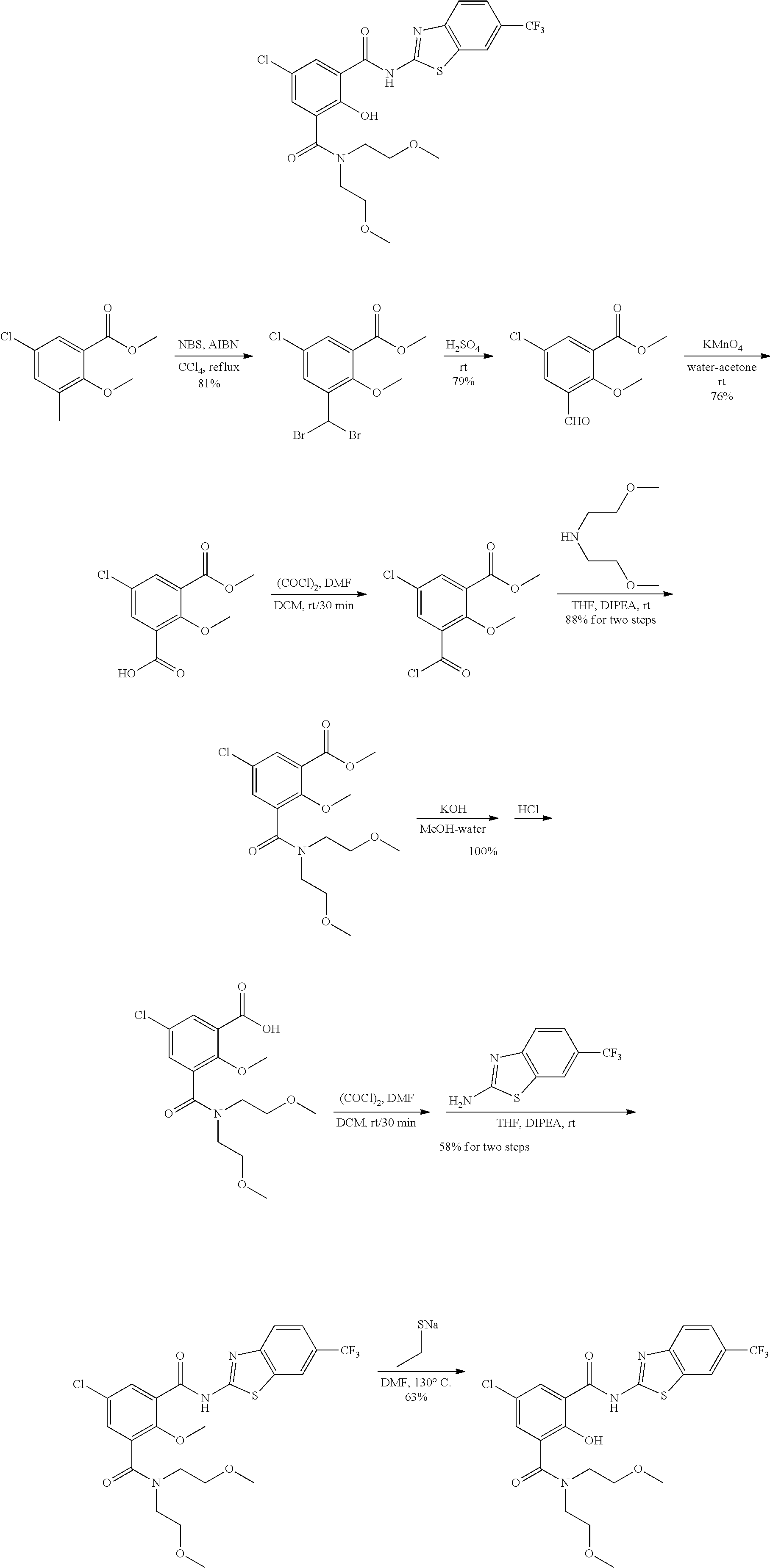

To a flame dried flask was added NBS (2.99 g, 16.79 mmol), AIBN (173 mg, 1.05 mmol) and a solution of methyl 5-chloro-2-methoxy-3-methylbenzoate (1.13 g, 5.26 mmol) in CCl4 (20 mL). The suspension was refluxed in the dark overnight. The mixture was cooled to rt and concentrated. The residue was purified via silica gel column chromatography to give methyl 5-chloro-3-(dibromomethyl)-2-methoxybenzoate as a yellow oil (1.59 g, 81%). ¹H NMR (300 MHz, cdcl₃) δ 8.04 (d, J=2.7 Hz, 1H), 7.80 (d, J=2.7 Hz, 1H), 7.09 (s, 1H), 3.95 (s, 6H).

Methyl 5-chloro-3-(dibromomethyl)-2-methoxybenzoate (1.59 g, 4.27 mmol) was dissolved in 10 mL concentrated sulfuric acid and the mixture was stirred at rt for 2 h. The reaction mixture was poured into ice water and extracted with ethyl acetate two times. The combined organic layer was washed with brine and dried over sodium sulfate. After concentration, the residue was purified via silica gel column chromatography to give methyl 5-chloro-3-formyl-2-methoxybenzoate (673 mg, 71%) as a white solid. ¹H NMR (300 MHz, cdcl₃) δ 10.33 (s, 1H), 8.00 (d, J=2.6 Hz, 1H), 7.91 (d, J=3.1 Hz, 1H), 3.98 (s, 4H), 3.94 (s, 4H).

To a stirred solution of methyl 5-chloro-3-formyl-2-methoxybenzoate (112 mg, 0.49 mmol) in acetone (3 mL) and water (3 mL) was added KMnO₄ (310 mg, 1.96 mmol). The resulting mixture was stirred at rt for 3.5 h. After completion of the reaction as indicated by TLC, 0.8 g sodium carbonate was added and stirred for another 15 minutes. The mixture was then filtered and the pH of the filtrate was adjusted to 2-3 when a large amount of solid was precipitated. The mixture was filtered and the solid was washed with water. The crude product was purified through recrystallization in ethanol to give 5-chloro-2-methoxy-3-(methoxycarbonyl)benzoic acid (90 mg, 76%) as a white solid. MS (ESI) [M+H]⁺ requires m/z 245.02, found m/z 245.0.

5-chloro-2-methoxy-3-(methoxycarbonyl)benzoic acid (90 mg, 0.37 mmol) was dissolved in DCM (3.0 mL), followed by the addition of a catalytic amount of DMF (1 drop) and oxalyl chloride (38 μL, 0.44 mmol) respectively. The reaction was allowed to stir at rt for 30 minutes and concentrated in vacuo. The residue was re-dissolved in THE (5.0 mL), and Hunig's base (192 μL, 1.1 mmol) and bis(2-methoxyethyl)amine (55 μL, 0.37 mmol) were added. The mixture was stirred at rt for 48 hours before silica gel was added to quench the reaction. The solvent was evaporated and the resulting residue was purified via silica gel column chromatography to yield methyl 3-(bis(2-methoxyethyl)carbamoyl)-5-chloro-2-methoxybenzoate (116 mg, 88%) as a colorless oil. ¹H NMR (300 MHz, cdcl₃) δ 7.81 (d, J=2.7 Hz, 1H), 7.42 (d, J=2.7 Hz, 1H), 4.04 (M, 1H), 3.93 (s, 3H), 3.88 (s, 3H), 3.71-3.62 (m, 2H), 3.57-3.40 (m, 3H), 3.39 (s, 3H), 3.37-3.24 (m, 2H), 3.23 (s, 3H). MS (ESI) [M+H]⁺ requires m/z 360.12, found m/z 360.10.

To a stirred solution of methyl 3-(bis(2-methoxyethyl)carbamoyl)-5-chloro-2-methoxybenzoate (116 mg, 0.323 mmol) in MeOH (3 mL) was added 2.0 mL 1N KOH solution. The resulting mixture was stirred at rt overnight. The solvent was evaporated out and the residue was portioned between ethyl acetate and 1N NaOH solution. The aqueous layer was acidified to pH=1 and extracted with ethyl acetate. This organic phase was dried over over sodium sulfate and concentrated in vacuo to afford 3-(bis(2-methoxyethyl)carbamoyl)-5-chloro-2-methoxybenzoic acid (115 mg, 100%) as a colorless oil which used in the next step without further purification. MS (ESI) [M+H]⁺ requires m/z 346.11, found m/z 346.20.

3-(bis(2-methoxyethyl)carbamoyl)-5-chloro-2-methoxybenzoic acid (115 mg, 0.32 mmol) was dissolved in DCM (3.0 mL), followed by the addition of a catalytic amount of DMF (1 drop) and oxalyl chloride (34 μL, 0.39 mmol) respectively. The reaction was allowed to stir at rt for 30 minutes and concentrated in vacuo. The residue was re-dissolved in THF (5.0 mL), and Hunig's base (170 μL, 0.98 mmol) and 6-(trifluoromethyl)benzo[d]thiazol-2-amine (71 mg, 0.32 mmol) were added. The mixture was stirred at rt for 48 hours before silica gel was added to quench the reaction. The solvent was evaporated and the resulting residue was purified via silica gel column chromatography to yield 5-chloro-2-methoxy-N¹,N¹-bis(2-methoxyethyl)-N³-(6-(trifluoromethyl)benzo[d]thiazol-2-yl)isophthalamide (95 mg, 58%) as a colorless oil. MS (ESI) [M+H]⁺ requires m/z 546.11, found m/z 546.20.

A solution of 5-chloro-2-methoxy-N¹,N¹-bis(2-methoxyethyl)-N³-(6-(trifluoromethyl)benzo[d]thiazol-2-yl)isophthalamide (95 mg, 0.174 mmol) in DMF (3 mL) was mixed with sodium ethanethiolate (74 mg, 0.872 mmol) and the resulting suspension was heated at 130° C. overnight. After completion of the reaction, 1N HCl was added to the reaction and extracted with ethyl acetate two times. The combined ethyl acetate layer was dried over sodium sulfate and concentrated under reduced pressure. The residue was purified via silica gel column chromatography to give 5-chloro-2-hydroxy-N¹,N¹-bis(2-methoxyethyl)-N³-(6-(trifluoromethyl)benzo[d]thiazol-2-yl)isophthalamide (55 mg, 63%) as a yellow solid. ¹H NMR (300 MHz, cdcl₃) δ 8.18-7.95 (m, 5H), 3.71 (brs, 4H), 3.56 (brs, 4H), 2.95 (s, 3H), 2.87 (s, 3H). MS (ESI) [M+H]⁺ requires m/z 532.09, found m/z 532.20.

### Example 42

### 5-chloro-N-(6-(difluoromethyl)benzo[d]thiazol-2-yl)-2-hydroxy-3-((2-methoxyethoxy)methyl)benzamide (42)

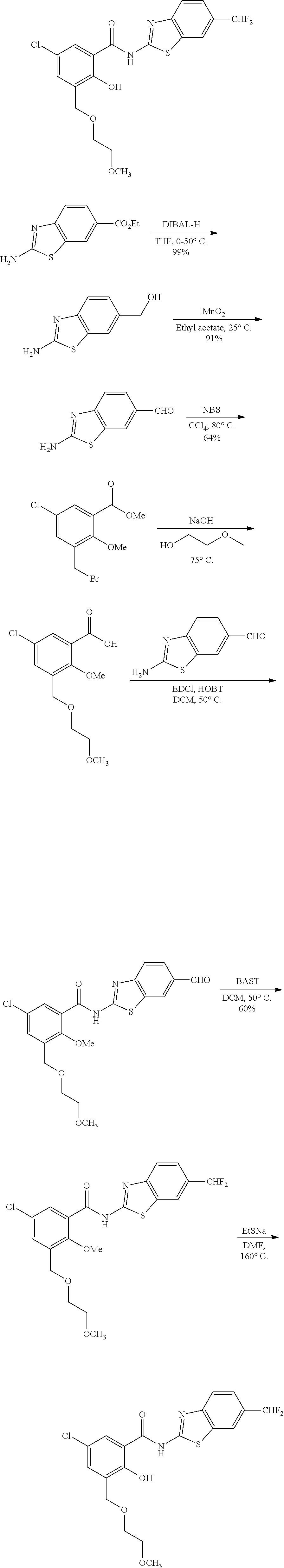

To a solution of ethyl 2-aminobenzo[d]thiazole-6-carboxylate (3 g, 13.50 mmol) in THF (60 mL) was added portion wise DIBAL-H (1 M, 53.99 mL) at 0° C. The reaction mixture was stirred at 50° C. for 12 h. The reaction mixture was quenched by addition saturated sodium potassium tartrate (30 mL), and then diluted with water (100 mL) and extracted with ethyl acetate (200 mL×3). The combined organic layer was washed with brine (200 mL), dried over Na₂SO₄, filtered and concentrated under reduced pressure to give (2-aminobenzo[d]thiazol-6-yl)methanol (2.4 g, crude) as a yellow solid. ¹H NMR (400 MHz, DMSO-d₆) δ ppm 7.57 (s, 1H), 7.38 (s, 2H), 7.27 (d, J=8.31 Hz, 1H), 7.14 (dd, J=8.07, 1.22 Hz, 1H), 5.09 (t, J=5.87 Hz, 1H), 4.47 (d, J=5.87 Hz, 2H). MS (ESI) [M+H]⁺ requires m/z 181.04, found m/z 181.2.

To a solution of (2-aminobenzo[d]thiazol-6-yl)methanol (1 g, 5.55 mmol) in ethyl acetate (30 mL) was added MnO₂ (4.8 g, 55.49 mmol). The mixture was stirred at 25° C. for 12 h. The reaction mixture was filtered with diatomite filter and concentrated under reduced pressure to give the crude product 2-aminobenzo[d]thiazole-6-carbaldehyde (900 mg, crude) as a yellow solid. ¹H NMR (400 MHz, DMSO-d₆) δ ppm 9.87 (s, 1H), 8.23 (d, J=1.47 Hz, 1H), 8.05 (br s, 2H), 7.75 (dd, J=8.31, 1.47 Hz, 1H), 7.44 (d, J=8.31 Hz, 1H). MS (ESI) [M+H]⁺ requires m/z 179.02, found m/z 179.1.

To a stirred solution of 5-chloro-2-methoxybenzoic acid (10 g, 53.59 mmol) in H₂SO₄ (18.25 mL, purity 98%) and TFA (36.50 mL) at 20° C. was added NBS (10.5 g, 58.95 mmol). The pale solution was stirred at 20° C. for 12 h. The resulting pale suspension was carefully poured onto crushed ice (100 mL). The mixture was extracted with ethyl acetate (50 mL×3), dried over Na₂SO₄ and concentrated under reduced pressure. The light yellow residue was suspend in DCM (50 mL). The solid was collected, washed with cold DCM (50 mL) and dried under vacuum to afford 3-bromo-5-chloro-2-methoxybenzoic acid (10 g, crude) as a white solid. ¹H NMR (400 MHz, DMSO-d₆) δ ppm 7.96 (t, J=2.32 Hz, 1H), 7.69 (t, J=2.32 Hz, 1H), 3.78 (d, J=2.43 Hz, 3H)

To a stirred solution of 3-bromo-5-chloro-2-methoxybenzoic acid (5 g, 18.83 mmol) in DMF (25 mL) was added K₂CO₃ (26 g, 188.33 mmol) followed by CH₃I (2.7 g, 18.83 mmol, 1.2 mL). The mixture was stirred at 20° C. for 12 h. H₂O (50 mL) was added and extracted with ethyl acetate (30 mL×2). The combined organic layer was washed with brine (100 mL) and dried over sodium sulfate. The organic layer was filtered and the solvent removed in vacuo to afford methyl 3-bromo-5-chloro-2-methoxybenzoate (4.7 g, purity 88.4%) as yellow oil. ¹H NMR (400 MHz, DMSO-d₆) δ ppm 8.03 (d, J=2.93 Hz, 1H), 7.74 (d, J=2.45 Hz, 1H), 3.87 (s, 3H), 3.81 (s, 3H). MS (ESI) [M+H]⁺ requires m/z 278.93, found m/z 281.0.

A mixture of methyl 3-bromo-5-chloro-2-methoxybenzoate (2 g, 7.16 mmol), potassium trifluoro(methyl)boranuide (1 g, 8.59 mmol), K₂CO₃ (3 g, 21.47 mmol), Pd(dppf)Cl₂ (1 g, 1.43 mmol) in dioxane (20 mL), H₂O (2 mL) was degassed and purged with N₂ 3 times, and then the mixture was stirred at 90° C. for 12 h under N₂ atmosphere. The reaction mixture was concentrated to remove solvent. The residue was purified by column chromatography (SiO₂, petroleum ether/ethyl acetate=1/0 to 10/1) to afford methyl 5-chloro-2-methoxy-3-methyl-benzoate (1.2 g, 5.31 mmol, 74.23% yield, 95% purity) as yellow oil.

To a flame dried flask was added NBS (1.2 g, 6.71 mmol), AIBN (91 mg, 559.06 μmol) and a solution of methyl 5-chloro-2-methoxy-3-methyl-benzoate (1.2 g, 5.59 mmol) in CCl₄ (10 mL). The suspension was stirred at 80° C. for 12 h. The reaction mixture was concentrated to remove solvent. The residue was purified by column chromatography (SiO₂, petroleum ether/ethyl acetate=1/0 to 100/1) to afford methyl 3-(bromomethyl)-5-chloro-2-methoxybenzoate (1.5 g, 3.58 mmol, 63.98% yield, 70% purity) as yellow oil. ¹H NMR (400 MHz, CHLOROFORM-d) δ ppm 7.69 (d, J=2.93 Hz, 1H), 7.46 (d, J=2.93 Hz, 1H), 4.44 (s, 2H), 3.88 (s, 3H), 3.86 (s, 3H).

To a stirred solution of methyl 3-(bromomethyl)-5-chloro-2-methoxybenzoate (1 g, 3.41 mmol) in 2-methoxyethanol (19.3 g, 253.63 mmol, 20 mL) was added 2N NaOH (15 mL). The resulting mixture was stirred at 75° C. for 12 h then concentrated in vacuo. The residue was diluted with water (5 mL) and adjusted to pH 9 with 2M NaOH, then extracted with ethyl acetate (5 mL×3). The clear solution was acidified with 1N HCl to pH 5 and then extracted with ethyl acetate (5 mL×3). The combined organic layer was washed with brine (20 mL) and dried over Na₂SO₄, filtered and concentrated under reduced pressure to afford 5-chloro-2-methoxy-3-((2-methoxyethoxy)methyl)benzoic acid (500 mg, purity:80%) as yellow oil. ¹H NMR (400 MHz, CHLOROFORM-d) δ ppm 7.90-8.00 (m, 1H), 7.63-7.73 (m, 1H), 4.63 (s, 2H), 3.92-3.94 (m, 3H), 3.72 (dd, J=5.62, 3.67 Hz, 2H), 3.59-3.65 (m, 2H), 3.42 (s, 3H). [M−H]⁻ requires m/z 273.0, found m/z 272.9.

To a solution of 2-aminobenzo[d]thiazole-6-carbaldehyde (162 mg, 910.09 μmol), 5-chloro-2-methoxy-3-((2-methoxyethoxy)methyl)benzoic acid (250 mg, 910.09 μmol) in DCM (4 mL) was added EDCI (209.36 mg, 1.09 mmol) and HOBt (184 mg, 1.37 mmol). The mixture was stirred at 50° C. for 12 h. The reaction mixture was concentrated under reduced pressure to remove solvent. The residue was purified by prep-HPLC (neutral condition) to afford 5-chloro-N-(6-formylbenzo[d]thiazol-2-yl)-2-methoxy-3-((2-methoxyethoxy)methyl)benzamide (80 mg, 97% purity) as a yellow solid. [M+H]⁺ requires m/z 435.07, found m/z 435.1

To a solution of 5-chloro-N-(6-formylbenzo[d]thiazol-2-yl)-2-methoxy-3-((2-methoxyethoxy)methyl)benzamide (50 mg, 114.97 μmol) in DCM (2 mL) was added BAST (406 mg, 1.84 mmol, 402.95 μL). The mixture was stirred at 50° C. for another 12 h. The reaction mixture was quenched by addition sat.NaHCO₃ (8 mL), and extracted with ethyl acetate (8 mL×3). The combined organic layer was washed with brine (10 mL), dried over Na₂SO₄, filtered and concentrated under reduced pressure to give a residue. The residue was purified by prep-TLC (SiO₂, petroleum ether/ethyl acetate=3:1) to afford 5-chloro-N-(6-(difluoromethyl)benzo[d]thiazol-2-yl)-2-methoxy-3-((2-methoxyethoxy)methyl)benzamide (40 mg, 72.67 μmol, 63.20% yield, 83% purity) as a white solid. [M+H]⁺ requires m/z 457.07, found m/z 457.2

To a solution of 5-chloro-N-(6-(difluoromethyl)benzo[d]thiazol-2-yl)-2-methoxy-3-((2-methoxyethoxy)methyl)benzamide (40 mg, 87.55 μmol) in DMF (2 mL) was added ethylsulfanylsodium (22 mg, 262.65 μmol). The mixture was stirred at 160° C. for 3 h. The reaction mixture was concentrated under reduced pressure to remove solvent. The residue was diluted with water (3 mL) and extracted with DCM (3 mL×3). The combined organic layers were washed with brine (5 mL), dried over Na₂SO₄, filtered and concentrated under reduced pressure to give a residue. The residue was purified by prep-TLC (SiO₂, Petroleum ether/Ethyl acetate=0:1). The residue was purified by prep-HPLC (HCl condition) to afford 5-chloro-N-(6-(difluoromethyl)benzo[d]thiazol-2-yl)-2-hydroxy-3-((2-methoxyethoxy)methyl)benzamide (5.22 mg, 99.157% purity) was obtained as a white solid. ¹H NMR (400 MHz, DMSO-d₆) δ ppm 8.28 (s, 1H), 7.99 (br s, 1H), 7.76 (br s, 1H), 7.67-7.73 (m, 1H), 7.52 (s, 1H), 6.98-7.33 (m, 1H), 4.56 (s, 2H), 3.65 (dd, J=5.62, 3.67 Hz, 2H), 3.52 (dd, J=5.62, 3.67 Hz, 2H), 3.28 (s, 3H). [M+H]⁺ requires m/z 443.06, found m/z 442.9

### Example 43

### 5-fluoro-2-hydroxy-3-((2-methoxyethoxy)methyl)-N-(6-(trifluoromethylbenzo[d]thiazol-2-yl)benzamide (43)

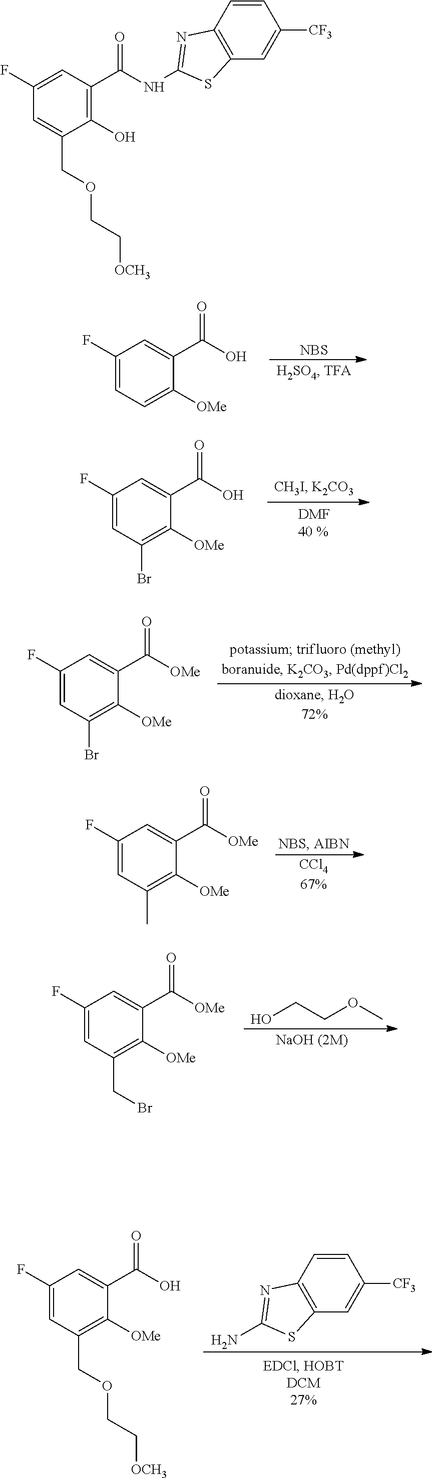

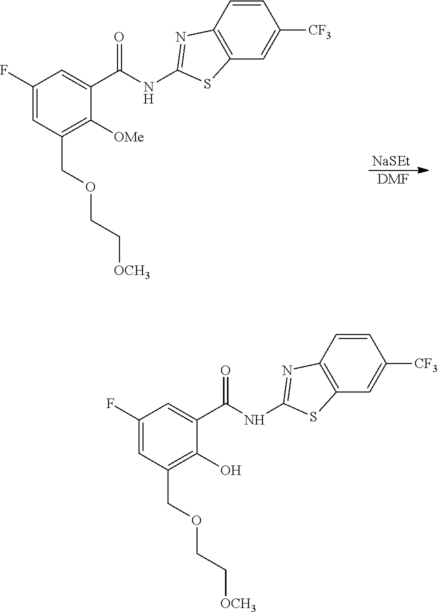

To a stirred solution of 5-fluoro-2-methoxybenzoic acid (5 g, 29.39 mmol) in H₂SO₄ (10 mL) (98%) and TFA (20 mL) at 20° C. was added NBS (5.7 g, 32.33 mmol). The pale solution was stirred at 20° C. for 12 h. The resulting pale suspension was carefully poured onto crushed ice (500 mL). The mixture was extracted with ethyl acetate (300 mL×3). The combined organic layer was washed with brine 1000 mL, dried over Na₂SO₄, filtered and concentrated under reduced pressure to afford 3-bromo-5-fluoro-2-methoxybenzoic acid (7 g) as a yellow solid. ¹H NMR (400 MHz, DMSO-d₆) δ ppm 7.77 (dd, J=7.89, 3.07 Hz, 1H), 7.50 (dd, J=8.77, 3.07 Hz, 1H), 3.76-3.80 (s, 3H).

To a stirred solution of 3-bromo-5-fluoro-2-methoxybenzoic acid (7 g, 28.11 mmol) in DMF (20 mL) was added K₂CO₃ (38.8 g, 281.09 mmol) followed by Mel (3.9 g, 28.11 mmol, 1.75 mL). The mixture was stirred at 20° C. for 12. Water (100 mL) was added and the extracted with ethyl acetate (150 mL×3). The combined organic layer was washed with brine (200 mL) and dried over sodium sulfate. The organic layer was filtered and the solvent was removed in vacuo. The residue was purified by column chromatography (SiO₂, petroleum ether/ethyl acetate 1/0 to 100/1) to afford methyl 3-bromo-5-fluoro-2-methoxybenzoate (4.6 g, 11.19 mmol, 39.81% yield, 64% purity) as colorless oil.

A mixture of methyl 3-bromo-5-fluoro-2-methoxybenzoate (3 g, 11.40 mmol), potassium;trifluoro(methyl)boranuide (1.5 g, 12.54 mmol), K₂CO₃ (4.7 g, 34.21 mmol) and Pd(dppf)Cl₂ (834 mg, 1.14 mmol) in dioxane (30 mL) and H₂O (3 mL) was degassed and purged with N₂ 3 times, then the mixture was stirred at 90° C. for 12 h under N₂ atmosphere. The reaction mixture was concentrated under reduced pressure to remove solvent. The residue was purified by column chromatography (SiO₂, petroleum ether/ethyl acetate 1/0 to 100/1) to afford methyl 5-fluoro-2-methoxy-3-methylbenzoate (1.8 g, 8.17 mmol, 71.67% yield, 90% purity) as yellow oil. ¹H NMR (400 MHz, CHLOROFORM-d) δ ppm 7.31 (dd, J=8.60, 2.87 Hz, 1H), 7.01-7.06 (m, 1H), 3.90 (s, 3H), 3.78 (s, 3H), 2.29 (s, 3H)

Methyl 5-fluoro-2-methoxy-3-methylbenzoate (1.8 g, 9.08 mmol), NBS (1.2 g, 7.27 mmol) and AIBN (149 mg, 908.22 umol) in CCl₄ (30 mL), were combined and the mixture was stirred at 80° C. for 12 h. The reaction mixture was concentrated under reduced pressure to remove solvent. The residue was purified by column chromatography (SiO₂, petroleum ether/ethyl acetate 1/0 to 100/1) to afford methyl 3-(bromomethyl)-5-fluoro-2-methoxybenzoate (2.1 g, 6.06 mmol, 66.76% yield, 80% purity) as yellow oil. ¹H NMR (400 MHz, CHLOROFORM-d) δ ppm 7.43 (dd, J=8.31, 3.42 Hz, 1H), 7.19-7.24 (m, 1H), 4.46 (s, 2H), 3.87 (s, 3H), 3.86 (s, 3H)

To methyl 3-(bromomethyl)-5-fluoro-2-methoxy-benzoate (2.1 g, 7.58 mmol) in NaOH (30 mL) (2M) was added 2-methoxyethanol (40.5 g, 532.63 mmol, 42.00 mL), the mixture was stirred at 75° C. for 12 h. The reaction mixture extracted with ethyl acetate (40 mL×3). The combined water layers were adjusted pH ~4 by addition of HCl (2N), and then the mixture was extracted with ethyl acetate (50 mL×3), the combined organic layer was concentrated under reduced pressure to afford 5-fluoro-2-methoxy-3-(2-methoxyethoxymethyl)benzoic acid (1.9 g, crude) as a yellow oil.

5-fluoro-2-methoxy-3-(2-methoxyethoxymethyl)benzoic acid (500 mg, 1.94 mmol), 6-(trifluoromethyl)benzo[d]thiazol-2-amine (422 mg, 1.94 mmol), EDCI (445 mg, 2.32 mmol) and HOBt (392 mg, 2.90 mmol) in DCM (15 mL), the mixture was stirred at 50° C. for 12 h. The reaction mixture was concentrated under reduced pressure to remove solvent. The residue was purified by prep-HPLC (neutral condition) to afford 5-Fluoro-2-methoxy-3-((2-methoxyethoxy)methyl))-N-[6-(trifluoromethyl)benzo[d]thiazol-2-yl]benzamide (300 mg, 523.53 umol, 27.04% yield, 80% purity) as a white solid.

5-Fluoro-2-methoxy-3-((2-methoxyethoxy)methyl))-N-[6-(trifluoromethyl)benzo[d]thiazol-2-yl]benzamide (50 mg, 109.07 μmol) and NaSEt (45 mg, 545.34 μmol) in DMF (2 mL), the mixture was stirred at 160° C. for 1 h. The reaction mixture was concentrated under reduced pressure to remove solvent. The residue was purified by prep-TLC (SiO₂, petroleum ether/ethyl acetate=1:1) and then the residue was purified by prep-HPLC (HCl condition) to afford 5-fluoro-2-hydroxy-3-((2-methoxyethoxy)methyl)-N-(6-(trifluoromethylbenzo[d]thiazol-2-yl)benzamide (13.33 mg, 30.00 μmol, 27.51% yield, 100% purity) as a white solid. ¹H NMR (400 MHz, METHANOL-d₄) δ ppm 8.27 (s, 1H), 7.71-7.85 (m, 3H), 7.40 (br d, J=6.85 Hz, 1H), 4.65 (s, 2H), 3.71-3.77 (m, 2H), 3.63 (dd, J=5.62, 3.67 Hz, 2H), 3.41 (s, 3H). MS (ESI) [M+H]⁺ requires m/z 445.0, found m/z 445.0

### Example 44

### 5-chloro-2-hydroxy-3-((2-methoxyethoxy)methyl)-N-(6-(trifluoromethyl)pyridin-3-yl)benzamide(44) and 5-chloro-2-hydroxy-3-((2-methoxyethoxy)methyl)-N-(6-(trifluoromethyl)pyridin-3-yl)benzamide hydrochloride (44A)

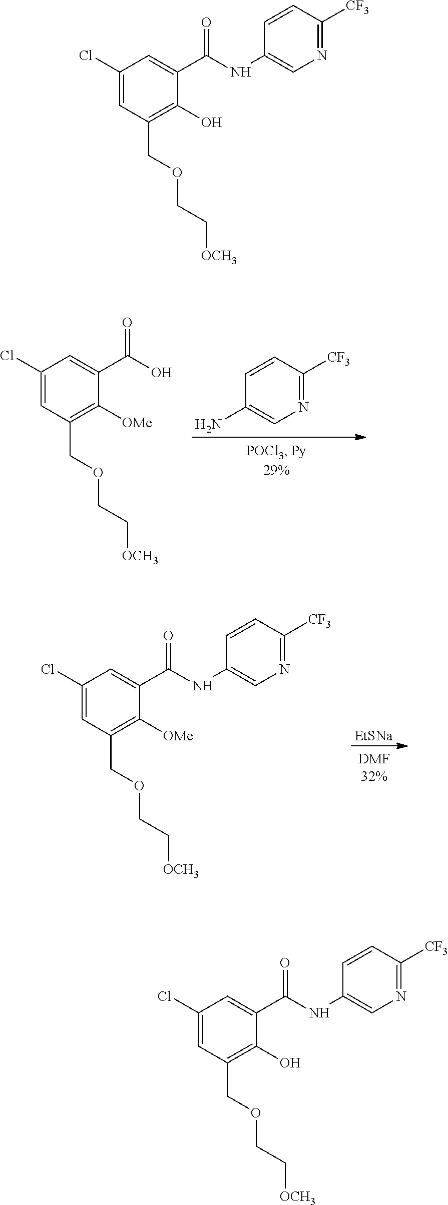

To a solution of 5-chloro-2-methoxy-3-(2-methoxyethoxymethyl)benzoic acid (200 mg, 728.08 μmol), 6-(trifluoromethyl)pyridin-3-amine (118 mg, 728.08 μmol) in Py (2 mL) was added POCl₃ (167 mg, 1.09 mmol, 101.49 μL) at 0° C. The mixture was stirred at 0° C. for 1 h. The reaction mixture was concentrated under reduced pressure to remove solvent. The residue was diluted with water (3 mL) and extracted with DCM (3 mL×3). The combined organic layer was washed with brine (5 mL), dried over Na₂SO₄, filtered and concentrated under reduced pressure to give a residue. The residue was purified by prep-TLC (SiO₂, petroleum ether/ethyl acetate 3/1) to afford 5-chloro-2-methoxy-3-((2-methoxyethoxy)methyl)-N-(6-(trifluoromethyl)pyridine-3-yl)benzamide (117 mg, 212.32 μmol, 29.16% yield, 76% purity) as a white solid. MS (ESI) [M+H]⁺ requires m/z 419.09, found m/z 419.2

To 5-chloro-2-methoxy-3-((2-methoxyethoxy)methyl)-N-(6-(trifluoromethyl)pyridine-3-yl)benzamide (95 mg, 226.84 μmol) in DMF (2 mL) was added NaSEt (38 mg, 453.68 umol) and the mixture was stirred at 160° C. for 2 h. The reaction mixture was concentrated under reduced pressure to remove solvent. The residue was purified by prep-HPLC (HCl condition) to afford 5-chloro-2-hydroxy-3-((2-methoxyethoxy)methyl)-N-(6-(trifluoromethyl)pyridin-3-yl)benzamide (32.99 mg, 73.67 μmol, 32.48% yield, 98.53% purity, HCl) as a brown solid. ¹H NMR (400 MHz, METHANOL-d₄) δ ppm 8.93 (d, J=2.19 Hz, 1H), 8.37 (dd, J=8.33, 2.19 Hz, 1H), 7.90 (d, J=2.19 Hz, 1H), 7.74 (d, J=8.77 Hz, 1H), 7.52 (d, J=2.63 Hz, 1H), 4.53 (s, 2H), 3.60-3.66 (m, 2H), 3.51-3.56 (m, 2H), 3.22 (dt, J=3.29, 1.43 Hz, 3H). MS (ESI) [M+H]⁺ requires m/z 405.08, found m/z 405.0

### Example 45

### 5-chloro-2-hydroxy-3-((2-methoxyethoxy)methyl)-N-(5-(trifluoromethyl)pyridin-2-yl)benzamide (45)

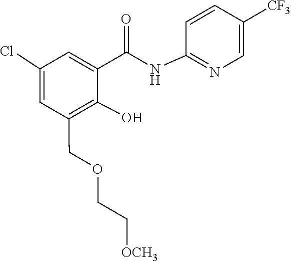 [SMILES: COCCOCc1cc(Cl)cc(C(=O)Nc2ccc(C(F)(F)F)cn2)c1O]

5-chloro-2-hydroxy-3-((2-methoxyethoxy)methyl)-N-(5-(trifluoromethyl)pyridin-2-yl)benzamide was made in a manner similar to Example 44. LCMS: MS (ESI) [M+H]⁺ requires m/z 405.08, found m/z 405.0. ¹H NMR (400 MHz, METHANOL-d₄) δ ppm 8.69 (s, 1H), 8.43 (d, J=8.82 Hz, 1H), 8.13 (dd, J=8.93, 2.09 Hz, 1H), 8.00 (d, J=2.43 Hz, 1H), 7.60 (d, J=2.43 Hz, 1H), 4.65 (s, 2H), 3.71-3.77 (m, 2H), 3.61-3.66 (m, 2H), 3.41 (s, 3H)

### Example 46

### 2-hydroxy-5-iodo-3-((2-methoxyethoxy)methyl)-N-(6-(trifluoromethyl)benzo[d]thiazol-2-yl)benzamide (46)

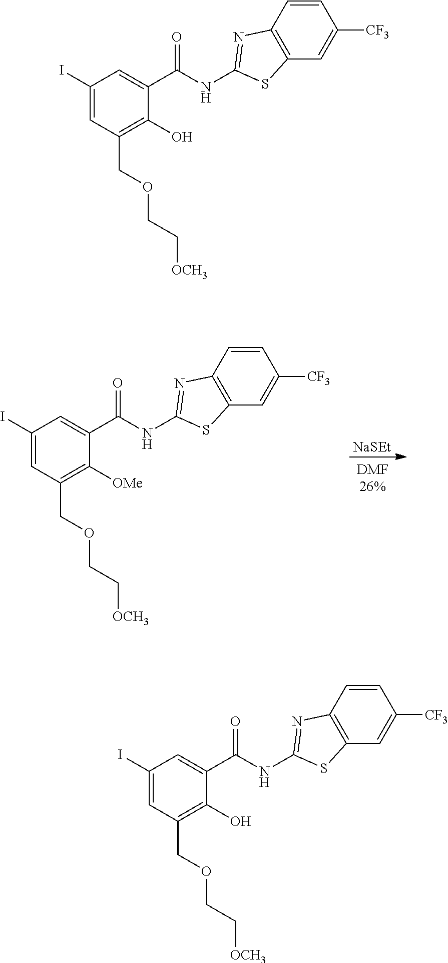 [SMILES: COCCOCc1cc(I)cc(C(=O)Nc2nc3ccc(C(F)(F)F)cc3s2)c1O ; COCCOCc1cc(I)cc(C(=O)Nc2nc3ccc(C(F)(F)F)cc3s2)c1OC ; COCCOCc1cc(I)cc(C(=O)Nc2nc3ccc(C(F)(F)F)cc3s2)c1O ; "NaSEt"]

5-Iodo-2-methoxy-3-((2-methoxyethoxy)methyl)-N-(6-(trifluoromethyl)benzo[d]thiazol-2-yl)benzamide (Example 48; 40 mg, 70.63 μmol, 1 eq) in DMF (2 mL) was stirred at 160° C. for 10 minutes, then NaSEt (29 mg, 353.15 μmol) was added, the mixture was stirred at 160° C. for 1 h. The reaction mixture was concentrated under reduced pressure to remove solvent. The residue was purified by prep-HPLC (HCl condition) to afford 2-hydroxy-5-iodo-3-((2-methoxyethoxy)methyl)-N-(6-(trifluoromethyl)benzo[d]thiazol-2-yl)benzamide (10.26 mg, 18.55 μmol, 26.26% yield, 99.848% purity) as a white solid. ¹H NMR (400 MHz, METHANOL-d₄) δ ppm 8.33 (d, J=1.96 Hz, 1H), 8.26 (s, 1H), 7.86 (s, 1H), 7.77 (br t, J=8.80 Hz, 2H), 4.62 (s, 2H), 3.72 (dd, J=5.62, 3.67 Hz, 2H), 3.62 (dd, J=5.62, 3.67 Hz, 2H), 3.39-3.42 (s, 3H). MS (ESI) [M+H]⁺ requires m/z 552.9, found m/z 553.0.

### Example 47

### 5-chloro-N-(2-chloro-6-(trifluoromethyl)pyridin-3-yl)-2-hydroxy-3-((2-methoxyethoxy)methyl)benzamide (47)

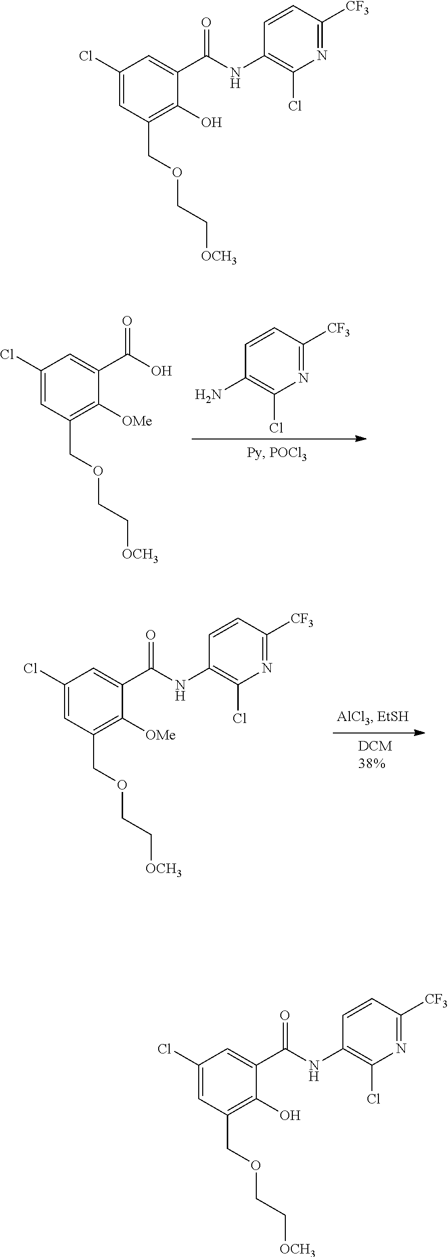 [SMILES: COCCOCc1cc(Cl)cc(C(=O)Nc2ccc(C(F)(F)F)nc2Cl)c1O ; COCCOCc1cc(Cl)cc(C(=O)O)c1OC ; Nc1ccc(C(F)(F)F)nc1Cl ; COCCOCc1cc(Cl)cc(C(=O)Nc2ccc(C(F)(F)F)nc2Cl)c1OC ; COCCOCc1cc(Cl)cc(C(=O)Nc2ccc(C(F)(F)F)nc2Cl)c1O]

To a solution of 5-chloro-2-methoxy-3-((2-methoxyethoxy)methyl)benzoic acid (50 mg, 182.02 μmol, Example 2), 2-chloro-6-(trifluoromethyl)pyridin-3-amine (35 mg, 182.02 μmol) in pyridine (2 mL) was added POCl₃ (41 mg, 273.03 μmol, 25.37 μL) at 0° C. The mixture was stirred at 20° C. for 1 h. The reaction mixture was concentrated under reduced pressure to remove solvent. The residue was purified by prep-TLC (SiO₂, petroleum ether/ethyl acetate=2:1) to afford 5-chloro-N-(2-chloro-6-(trifluoromethyl)pyridine-3yl)-2-methoxy-3-((2-methoxyethoxy)methyl)benzamide (75 mg, 65% purity) as a white solid.

To a mixture of EtSH (1 mL) and DCM (1 mL) was added AlCl₃ (66 mg, 496.43 μmol, 27.13 μL) at 0° C., the resulting solution was warmed to 20° C., and then 5-chloro-N-(2-chloro-6-(trifluoromethyl)pyridine-3yl)-2-methoxy-3-((2-methoxyethoxy)methyl)benzamide (75 mg, 165.48 μmol) was added with stirring and the mixture was stirred at 20° C. for 9 hs. The mixture was poured into 2 mL of water, acidified with HCl to pH ~4-5, then extracted with DCM (2×3), dried over Na₂SO₄, filtered and concentrated under reduced pressure to give a residue. The residue was purified by prep-HPLC (HCl condition) to afford 5-chloro-N-(2-chloro-6-(trifluoromethyl)pyridin-3-yl)-2-hydroxy-3-((2-methoxyethoxy)methyl)benzamide (29.94 mg, 62.94 μmol, 38.04% yield, 100% purity, HCl) as a white solid. ¹H NMR (400 MHz, METHANOL-d₄) δ ppm 8.90 (br d, J=8.31 Hz, 1H), 8.02 (br s, 1H), 7.85 (br d, J=8.31 Hz, 1H), 7.56 (br s, 1H), 4.69 (s, 2 H), 3.75 (br s, 2H), 3.64 (br s, 2H), 3.42 (s, 3H). MS (ESI) [M+H]⁺ requires m/z 439.04, found m/z 439.0

### Example 48 and 49

### 5-iodo-2-methoxy-3-((2-methoxyethoxy)methyl)-N-(6-(trifluoromethylbenzo[d]thiazol-2-yl]benzamide (48) 2-hydroxy-3-((2-methoxyethoxy)methyl)-5-methyl-N-(6-(trifluoromethyl)benzo[d]thiazol-2-yl)benzamide (49A) 2-hydroxy-3-((2-methoxyethoxy)methyl)-N-(6-(trifluoromethyl)benzo[d]thiazol-2-yl)benzamide (49B)

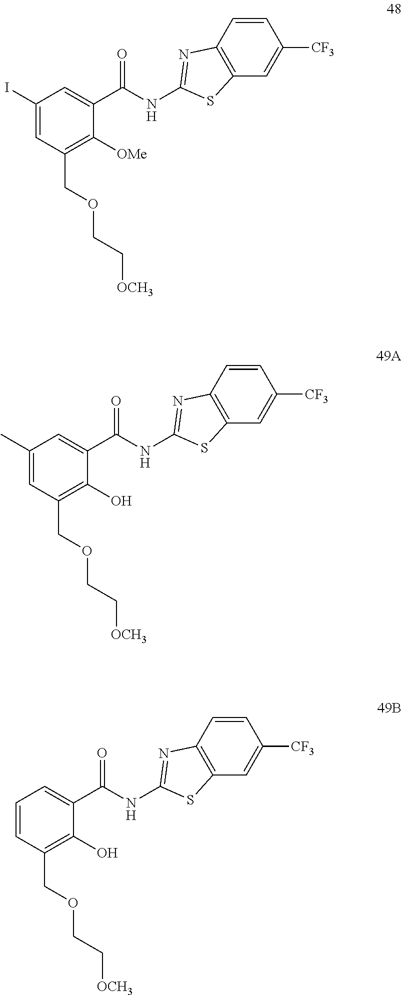 [SMILES: COCCOCc1cc(I)cc(C(=O)Nc2nc3ccc(C(F)(F)F)cc3s2)c1OC ; COCCOCc1cc(C)cc(C(=O)Nc2nc3ccc(C(F)(F)F)cc3s2)c1O ; COCCOCc1cccc(C(=O)Nc2nc3ccc(C(F)(F)F)cc3s2)c1O]

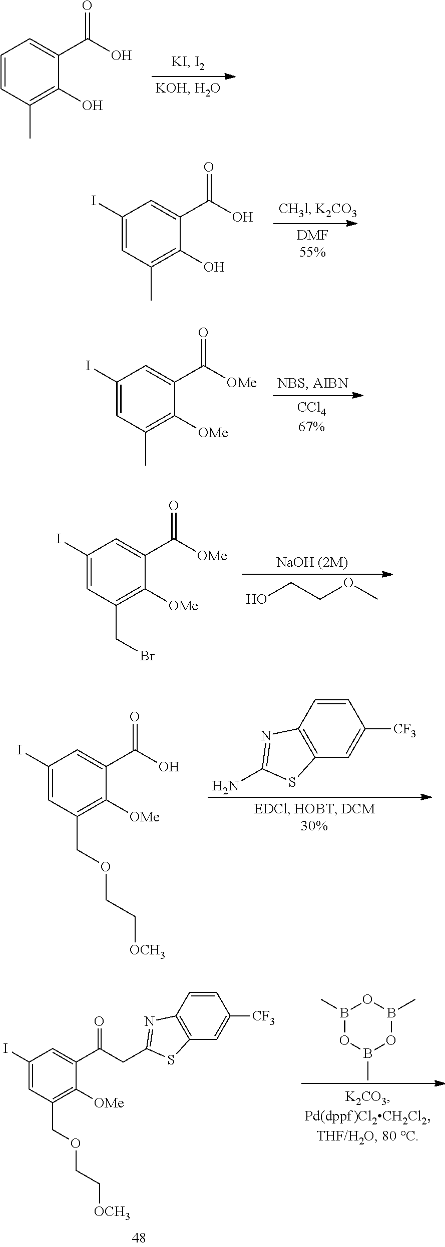

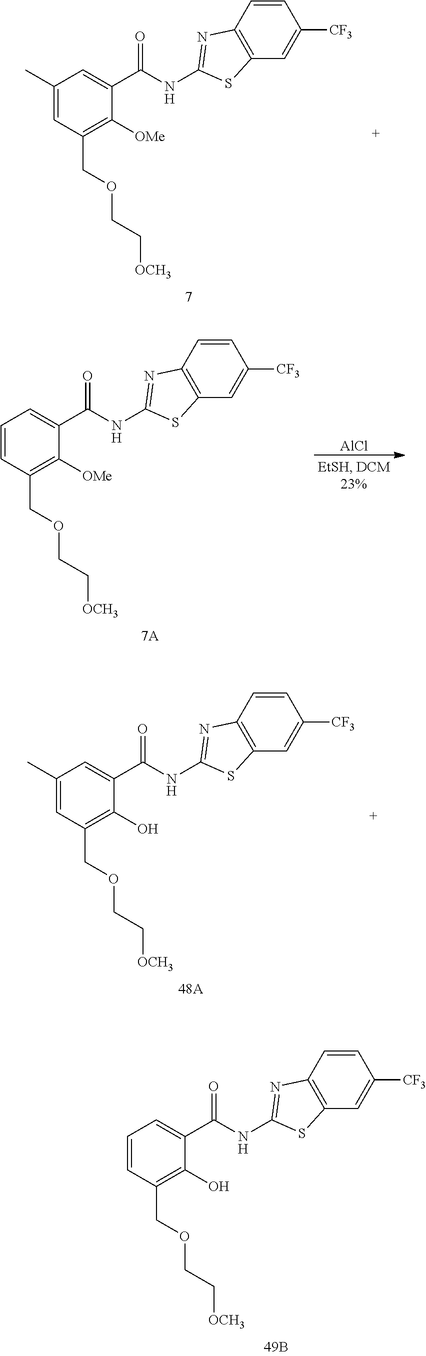 [SMILES: COCCOCc1cc(C)cc(C(=O)Nc2nc3ccc(C(F)(F)F)cc3s2)c1OC ; COCCOCc1cccc(C(=O)Nc2nc3ccc(C(F)(F)F)cc3s2)c1OC ; "AlCl" ; COCCOCc1cc(C)cc(C(=O)Nc2nc3ccc(C(F)(F)F)cc3s2)c1O ; COCCOCc1cccc(C(=O)Nc2nc3ccc(C(F)(F)F)cc3s2)c1O]

I₂ (50 g, 197.18 mmol, 39.7 mL) and KI (27.2 g, 164.31 mmol) in H₂O (125 mL) were slowly added to a 0° C. mixture of 2-hydroxy-3-methylbenzoic acid (25 g, 164.31 mmol) in KOH (200 mL, 10%), and the mixture was stirred at 0° C. for 1 h. The reaction mixture was diluted with water (500 mL) and extracted with ethyl acetate (1000 mL×3). The clear solution was acidified with 1N HCl to pH 5 and then extracted with ethyl acetate (1000 mL×3). The combined organic layer was washed with brine (2000 mL), dried over Na₂SO₄, filtered and concentrated under reduced pressure to afford 2-hydroxy-5-iodo-3-methylbenzoic acid (17 g, crude) as a yellow solid.

2-hydroxy-5-iodo-3-methylbenzoic acid (7 g, 25.18 mmol) and K₂CO₃ (34.7 g, 251.76 mmol) in DMF (140 mL) was stirred at 20° C. for 30 minutes, then Mel (7.8 g, 55.39 mmol, 3.45 mL) was added dropwise, and the mixture was stirred at 20° C. for another 12 h. H₂O (300 mL) was added and extracted with ethyl acetate (200 mL×3). The combined organic layer was washed with brine (500 mL×2), and dried over sodium sulfate. The organic layer was filtered and the solvent removed in vacuo to afford methyl 5-iodo-2-methoxy-3-methylbenzoate (5 g, 13.88 mmol, 55.15% yield, 85% purity) as a yellow solid.

To methyl 5-iodo-2-methoxy-3-methylbenzoate (5 g, 16.33 mmol) and NBS (2.9 g, 16.33 mmol) in CCl₄ (80 mL) was added AIBN (268 mg, 1.63 mmol), and the mixture was stirred at 80° C. for 12 h. The reaction mixture was concentrated under reduced pressure to remove solvent. The residue was purified by column chromatography (SiO₂, petroleum ether/ethyl acetate=1/0 to 20/1) to afford methyl 3-(bromomethyl)-5-iodo-2-methoxybenzoate (5.8 g, 11.00 mmol, 67.33% yield, 73% purity) as a yellow solid.

To a stirred solution of methyl 3-(bromomethyl)-5-iodo-2-methoxybenzoate (5.3 g, 13.77 mmol) in 2-methoxyethanol (77.9 g, 1.02 mol, 80.82 mL), was added 2N NaOH (50 mL). The resulting mixture was stirred at 75° C. for 12 h. The residue was diluted with water (300 mL) and extracted with ethyl acetate (300 mL×3). The clear solution was acidified with 1N HCl to pH ~5 and then extracted with ethyl acetate (200 mL×3). The combined organic layer was washed with brine (500 mL), dried over Na₂SO₄, filtered and concentrated under reduced pressure to afford 5-iodo-2-methoxy-3-((2 methoxyethoxy)methyl)benzoic acid (4.6 g, crude) as yellow oil.

To a solution of 6-(trifluoromethyl)-benzo[d]thiazol-2-amine (1.4 g, 6.55 mmol), 5-iodo-2-methoxy-3-((2-methoxyethoxy)methyl)benzoic acid (2 g, 5.46 mmol) in DCM (60 mL) was added EDCI (1.2 g, 6.55 mmol) and HOBt (1.1 g, 8.19 mmol). The mixture was stirred at 50° C. for 12 h. The reaction mixture was concentrated under reduced pressure to remove solvent. The residue was purified by prep-HPLC (neutral condition) to afford 5-iodo-2-methoxy-3-((2-methoxyethoxy)methyl)-N-(6-(trifluoromethylbenzo[d]thiazol-2-yl]benzamide (920 mg, 1.62 mmol, 29.74% yield, 100% purity) as a white solid.

A mixture of 2,4,6-trimethyl-1,3,5,2,4,6-trioxatriborinane (199 mg, 794.59 μmol, 222.16 μL) 50% in THF, 5-iodo-2-methoxy-3-((2-methoxyethoxy)methyl)-N-(6-(trifluoromethylbenzo[d]thiazol-2-yl]benzamide (150 mg, 264.86 μmol), K₂CO₃ (73 mg, 529.73 μmol) and Pd(dppf)Cl₂—CH₂Cl₂ (21 mg, 26.49 μmol) in H₂O (0.5 mL) and THF (2 mL), and the mixture was stirred at 80° C. for 12 h under N₂ atmosphere. The reaction mixture was concentrated under reduced pressure to remove solvent. The residue was purified by prep-TLC (SiO₂, petroleum ether/ethyl acetate 0/1) to afford the mixture of 2-methoxy-3-((2-methoxyethoxy)methyl)-5-methyl-N-(6-(trifluoromethyl)benzo[d]thiazol-2-yl)benzamide and 2-methoxy-3-((2-methoxyethoxy)methyl)-N-(6-(trifluoromethyl)benzo[d]thiazol-2-yl)benzamide (50 mg) as a white solid.

To a mixture of EtSH (1 mL) and DCM (1 mL) was added AlCl₃ (26 mg, 198.04 μmol, 10.82 μL) at 0° C., the resulting solution was warmed to 20° C., and then the mixture 2-methoxy-3-((2-methoxyethoxy)methyl)-5-methyl-N-(6-(trifluoromethyl)benzo[d]thiazol-2-yl)benzamide and 2-methoxy-3-((2-methoxyethoxy)methyl)-N-(6-(trifluoromethyl)benzo[d]thiazol-2-yl)benzamide (50 mg) was added with stirring, and the mixture was stirred at 20° C. for 9 h. The mixture was poured into water (2 mL), HCl was added (2N) to reach a pH ~4-5, and the mixture was extracted with DCM (3 mL×2). The combined organic layer was washed with brine (5 mL), dried over Na₂SO₄, filtered and concentrated under reduced pressure to give a residue. The residue was purified by prep-HPLC (HCl condition) to afford 2-hydroxy-3-((2-methoxyethoxy)methyl)-5-methyl-N-(6-(trifluoromethyl)benzo[d]thiazol-2-yl)benzamide (5.15 mg, 11.69 μmol, 17.71% yield, 100% purity) as a black solid. ¹H NMR (400 MHz, METHANOL-d₄) δ ppm 8.28 (s, 1H), 7.84 (s, 2H), 7.73 (d, J=8.80 Hz, 1H), 7.44 (s, 1H), 4.65 (s, 2H), 3.68-3.74 (m, 2H), 3.59-3.64 (m, 2H), 3.40 (s, 3H), 2.35 (s, 3H). MS (ESI) [M+H]⁺ requires m/z 441.10, found m/z 441.1 and 2-methoxy-3-((2-methoxyethoxy)methyl)-5-methyl-N-(6-(trifluoromethyl)benzo[d]thiazol-2-yl)benzamide (6.62 mg, 15.22 μmol, 23.06% yield, 98.04% purity) as a white solid. ¹H NMR (400 MHz, METHANOL-d₄) δ ppm 8.28 (s, 1H), 8.00-8.06 (m, 1H), 7.86 (br s, 1H), 7.74 (d, J=8.80 Hz, 1H), 7.61 (br d, J=7.34 Hz, 1H), 7.02 (br t, J=7.58 Hz, 1H), 4.68 (s, 2H), 3.69-3.75 (m, 2H), 3.59-3.64 (m, 2H), 3.40 (s, 3H). MS (ESI) [M+H]⁺ requires m/z 427.09, found m/z 427.0.

### Example 50

### 5-chloro-2-hydroxy-3-methoxy-N-(5-(trifluoromethyl)thiazol-2-yl)benzamide (50)

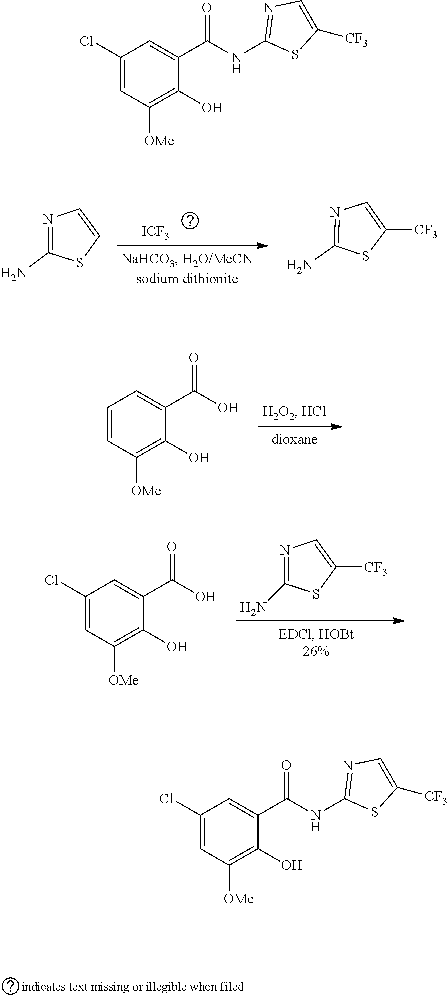

Sodium dithionite (5.2 g, 29.96 mmol, 6.52 mL) was added in one portion to a mixture of thiazol-2-amine (3 g, 29.96 mmol), NaHCO₃ (2.5 g, 29.96 mmol, 1.17 mL) and trifluoro(iodo)methane (23.4 g, 29.96 mmol) in ACN (40 mL) and H₂O (10 mL) at 5° C. and stirred for 12 h under N₂ atmosphere. The reaction mixture was concentrated under reduced pressure to remove solvent. The residue was diluted with water 20 mL and extracted with ethyl acetate (10 mL×3). The combined organic layer was washed with brine (50 mL), dried over Na₂SO₄, filtered and concentrated under reduced pressure to give a residue. The residue was purified by column chromatography (SiO₂, petroleum ether/ethyl acetate=1/0 to 10/1) to afford 5-(trifluoromethyl)thiazol-2-amine (790 mg, 4.32 mmol, 14.43% yield, 92% purity) as yellow oil.

To a solution of 2-hydroxy-3-methoxybenzoic acid (2 g, 11.89 mmol) in dioxane (20 mL), was added HCl (6 M, 2.08 mL). H₂O₂ (1.4 g, 13.08 mmol, 1.26 mL, 30% purity) was added with rapid stirring of 1 min, and then the mixture was stirred at 20° C. for 12 h, producted from light. The reaction mixture was quenched by addition 10% Na₂SO₃ (10 mL), concentrated under reduced pressure to remove solvent, then diluted with water (10 mL) and adjusted to pH 9 by addition of 2N NaOH. Then the mixture was extracted with ethyl acetate (15 mL×3). The clear solution was acidified to pH 5 with 1N HCl and then extracted with ethyl acetate (15 mL×3). The combined organic layer was washed with brine (50 mL), dried over Na₂SO₄, filtered and concentrated under reduced pressure to give 5-chloro-2-hydroxy-3-methoxybenzoic acid (2.1 g, crude) as a yellow solid.

To 5-chloro-2-hydroxy-3-methoxybenzoic acid (50 mg, 246.80 μmol) and 5-(trifluoromethyl) thiazol-2-amine (41 mg, 246.80 μmol) in DCM (3 mL) was added EDCI (56 mg, 296.16 μmol) and HOBt (50 mg, 370.20 μmol), and the mixture was stirred at 45° C. for 12 h. The reaction mixture was concentrated under reduced pressure to remove solvent. The residue was purified by prep-HPLC (HCl condition) to afford 5-chloro-2-hydroxy-3-methoxy-N-(5-(trifluoromethyl)thiazol-2-yl)benzamide (23.11 mg, 64.18 μmol, 26.00% yield, 97.95% purity) as a white solid. ¹H NMR (400 MHz, METHANOL-d₄) δ ppm 7.95 (d, J=1.32 Hz, 1H), 7.62 (d, J=2.43 Hz, 1H), 7.23 (d, J=2.43 Hz, 1H), 3.95 (s, 3H). MS (ESI) [M+H]⁺ requires m/z 352.9, found m/z 353.0

### Example 51

### 5-chloro-2-hydroxy-3-(morpholinomethyl)-N-(5-(trifluoromethyl)thiazol-2-yl)benzamide (51)

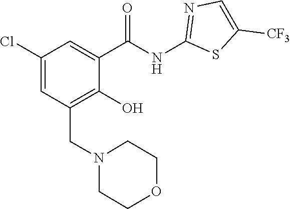 [SMILES: O=C(Nc1ncc(C(F)(F)F)s1)c1cc(Cl)cc(CN2CCOCC2)c1O]

5-chloro-2-hydroxy-3-(morpholinomethyl)-N-(5-(trifluoromethyl)thiazol-2-yl)benzamide was made in a similar manner as Example 22. LCMS: MS (ESI) [M+H]⁺ requires m/z 422.0, found m/z 422.0. ¹HNMR (400 MHz, METHANOL-d₄) δ ppm 8.14 (d, J=2.45 Hz, 1H), 8.00 (d, J=1.47 Hz, 1H), 7.59 (d, J=2.45 Hz, 1H), 4.35 (s, 2H), 3.90 (br s, 4H), 3.28 (br d, J=4.40 Hz, 4H)

### Example 52

### 5-chloro-2-hydroxy-3-(pyrrolidin-1-ylmethyl)-N-(5-(trifluoromethyl)thiazol-2-yl)benzamide (52)

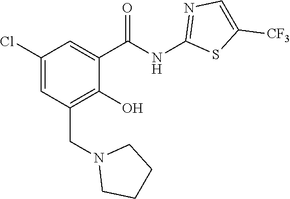 [SMILES: O=C(Nc1ncc(C(F)(F)F)s1)c1cc(Cl)cc(CN2CCCC2)c1O]

5-chloro-2-hydroxy-3-(pyrrolidin-1-ylmethyl)-N-(5-(trifluoromethyl)thiazol-2-yl)benzamide was made in a similar manner to Example 22. LCMS: MS (ESI) [M+H]⁺ requires m/z 406.0, found m/z 406.0. ¹HNMR (400 MHz, METHANOL-d4) δ ppm 8.25 (d, J=2.65 Hz, 1H), 8.07 (d, J=1.10 Hz, 1H), 7.74 (d, J=2.43 Hz, 1H), 4.46 (s, 2H), 3.56 (br s, 2H), 3.22-3.30 (m, 2H), 2.21 (br s, 2H), 2.05 (br s, 2H)

### Example 53

### 5-chloro-2-hydroxy-3-(morpholinomethyl)-N-(6-(trifluoromethyl)pyridin-3-yl)benzamide (53) and 5-chloro-2-hydroxy-3-(morpholinomethyl)-N-(6-(trifluoromethyl)pyridin-3-yl)benzamide hydrochloride (53A)

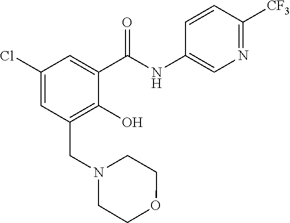 [SMILES: O=C(Nc1ccc(C(F)(F)F)nc1)c1cc(Cl)cc(CN2CCOCC2)c1O]

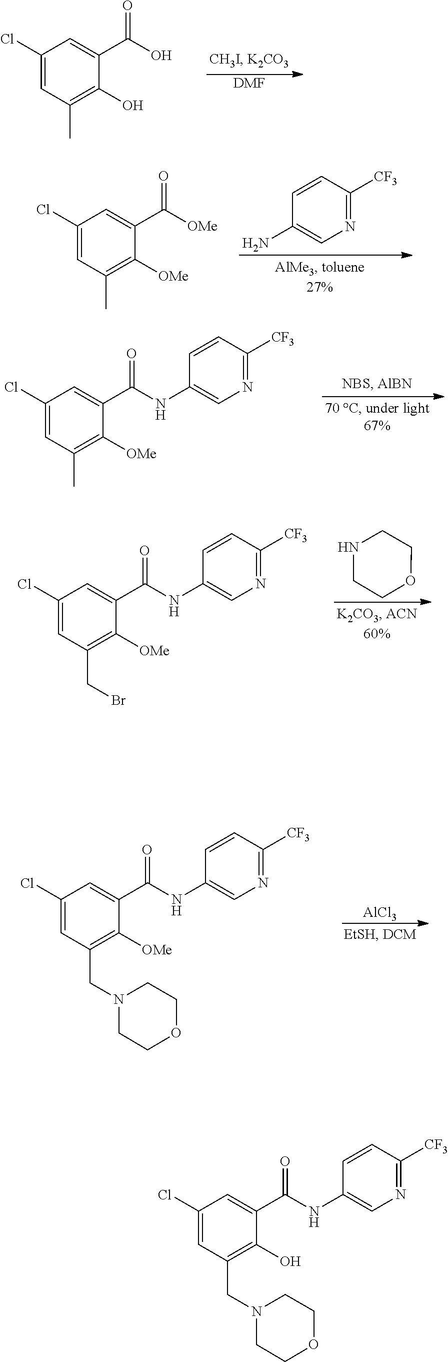

To a stirred solution of 5-chloro-2-hydroxy-3-methylbenzoic acid (7 g, 37.51 mmol) in DMF (200 mL) was added K₂CO₃ (51.8 g, 375.15 mmol) followed by Mel (11.7 g, 82.53 mmol, 5.14 mL). The mixture was stirred at 20° C. for 12 h. H₂O (500 mL) was added and extracted with ethyl acetate (300 mL×3). The combined organic layer was washed with brine (500 mL) and dried over sodium sulfate. The organic layer was filtered and the solvent removed in vacuo. The residue was purified by column chromatography (SiO₂, petroleum ether/ethyl acetate=1/0 to 10/1) to afford methyl 5-chloro-2-methoxy-3-methylbenzoate (87% purity) 11.2 g as a yellow solid.

Methyl 5-chloro-2-methoxy-3-methylbenzoate (2 g, 9.32 mmol) and 6-(trifluoromethyl)pyridin-3-amine (1.5 g, 9.32 mmol) in toluene (40 mL), was stirred at 0° C., then AlMe₃ (2 M, 13.98 mL) was added at 0° C., the mixture was stirred at 20° C. for another 12 h. The reaction mixture was added to sat. NH₄Cl (100 mL), and then extracted with ethyl acetate (40 mL×3). The combined organic layer was washed with brine (100 mL×3), dried over Na₂SO₄, filtered and concentrated under reduced pressure to give a residue. The residue was purified by column chromatography (SiO₂, petroleum ether/Ethyl acetate=1/0 to 20/1) to afford 5-chloro-2-methoxy-3-methyl-N-(6-(trifluoromethylpyridin-3-yl)benzamide (1.6 g, 2.51 mmol, 26.90% yield, 54% purity) as a yellow solid.

To 5-chloro-2-methoxy-3-methyl-N-(6-(trifluoromethylpyridin-3-yl)benzamide (1.5 g, 4.35 mmol) in CCl₄ (20 mL) was added NBS (851 mg, 4.79 mmol) and AIBN (714 mg, 4.35 mmol), and the mixture was stirred at 70° C. for 12 (under light, 1000 W). The reaction mixture was concentrated under reduced pressure to remove solvent. The residue was purified by column chromatography (SiO₂, petroleum ether/ethyl acetate=50/1 to 10/1) to afford 3-(bromomethyl)-5-chloro-2-methoxy-N-(6-(trifluoromethyl)pyridine-3-yl)benzamide (1.4 g, 2.91 mmol, 66.84% yield, 88% purity) as a white solid.

To morpholine (113 mg, 1.30 mmol, 114.26 μL) in ACN (10 mL), was added K₂CO₃ (326 mg, 2.36 mmol). The mixture was stirred at 0° C. for 10 minutes, then 3-(bromomethyl)-5-chloro-2-methoxy-N-(6-(trifluoromethyl)pyridine-3-yl)benzamide (500 mg, 1.18 mmol) was added, and the mixture was stirred at 30° C. for 12 h. The reaction mixture was concentrated under reduced pressure to remove solvent. The residue was purified by column chromatography (SiO₂, petroleum ether/ethyl acetate=1/0 to 1/1) to afford 5-chloro-2-methoxy-3-(morpholinomethyl)-N-(6-(trifluoromethyl)pyridin-3-yl)benzamide (340 mg, 711.93 μmol, 60.32% yield, 90% purity) as yellow oil.

AlCl₃ (139 mg, 1.05 mmol, 57.21 μL) was added to a mixture of EtSH (2 mL) and DCM (2 mL) at 0° C., then stirred at 0° C. for 10 minutes. 5-Chloro-2-methoxy-3-(morpholinomethyl)-N-(6-(trifluoromethyl)pyridin-3-yl)benzamide (150 mg, 348.98 μmol) was added, and the mixture was stirred at 20° C. for 2 h. The reaction mixture was concentrated under reduced pressure to remove solvent. The residue was purified by prep-HPLC (HCl condition) to afford 5-chloro-2-hyoxy-3-(morpholinomethyl)-N-(6-(trifluoromethyl)pyridin-3-yl)benzamide (98.15% purity, HCl) 61.99 mg as a white solid. ¹H NMR (400 MHz, DMSO-d₆) δ ppm 9.06 (s, 1H), 8.43 (dd, J=8.80, 1.96 Hz, 1H), 8.30 (br s, 1H), 7.98 (d, J=8.80 Hz, 1H), 7.86 (br s, 1H), 4.35 (br s, 2H), 3.83 (br d, J=9.29 Hz, 4H), 3.16-3.32 (m, 2H). MS (ESI) [M+H]⁺ requires m/z 416.0, found m/z 416.0.

### Example 54

### 5-chloro-2-hydroxy-3-(piperidin-1-ylmethyl)-N-(6-(trifluoromethyl)pyridin-3-yl)benzamide (54) and 5-chloro-2-hydroxy-3-(piperidin-1-ylmethyl)-N-(6-(trifluoromethyl)pyridin-3-yl)benzamide hydrochloride (54A)

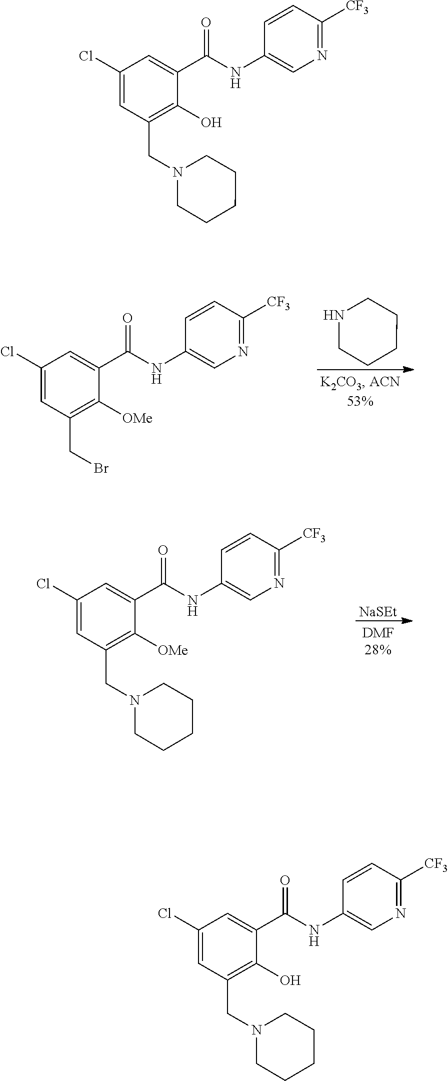 [SMILES: O=C(Nc1ccc(C(F)(F)F)nc1)c1cc(Cl)cc(CN2CCCCC2)c1O ; COc1c(CBr)cc(Cl)cc1C(=O)Nc1ccc(C(F)(F)F)nc1 ; C1CCNCC1 ; COc1c(CN2CCCCC2)cc(Cl)cc1C(=O)Nc1ccc(C(F)(F)F)nc1 ; O=C(Nc1ccc(C(F)(F)F)nc1)c1cc(Cl)cc(CN2CCCCC2)c1O ; "NaSEt"]

To piperidine (30 mg, 354.10 μmol, 34.97 μL, Example 53) in ACN (2 mL) was added K₂CO₃ (97 mg, 708.20 μmol), and the mixture was stirred at 0° C. for 10 minutes. 3-(Bromomethyl)-5-chloro-2-methoxy-N-(6-(trifluoromethyl)pyridine-3-yl)benzamide (150 mg, 354.10 μmol) was added, and the mixture was stirred at 30° C. for 12 h. The reaction mixture was concentrated under reduced pressure to remove solvent. The residue was purified by prep-TLC (SiO₂, petroleum ether/ethyl acetate=1:1) to afford 5-chloro-2-methoxy-3-(piperidin-1-ylmethyl)-N-(6-(trifluoromethyl)pyridin-3-yl)benzamide (100 mg, 186.98 μmol, 52.81% yield, 80% purity) as yellow oil.

5-chloro-2-methoxy-3-(piperidin-1-ylmethyl)-N-(6-(trifluoromethyl)pyridin-3-yl)benzamide (60 mg, 140.24 μmol) in DMF (2 mL) was stirred at 160° C. for 10 minutes, then NaSEt (58 mg, 701.19 μmol) was added, and the mixture was stirred at 160° C. for 1 h. The reaction mixture was concentrated under reduced pressure to remove solvent. The residue was purified by prep-HPLC (HCl condition) to afford 5-chloro-2-hydroxy-3-(piperidin-1-ylmethyl)-N-(6-(trifluoromethyl)pyridin-3-yl)benzamide (18 mg, 39.32 μmol, 28.04% yield, 95.24% purity, HCl) as a white solid. ¹H NMR (400 MHz, METHANOL-d₄) δ ppm 9.07 (d, J=2.21 Hz, 1H), 8.47 (dd, J=8.49, 2.32 Hz, 1H), 8.30 (d, J=2.43 Hz, 1H), 7.88 (d, J=8.60 Hz, 1H), 7.79 (d, J=2.43 Hz, 1H), 4.38 (s, 2H), 3.52 (br d, J=12.35 Hz, 2H), 3.10 (br t, J=12.35 Hz, 2H), 1.97 (br d, J=14.55 Hz, 2H), 1.70-1.87 (m, 3H), 1.47-1.62 (m, 1H). MS (ESI) [M+H]⁺ requires m/z 414.1, found m/z 414.1.

### Example 55

### 5-chloro-3-((3-cyanopyrrolidin-1-yl)methyl)-2-hydroxy-N-(6-(trifluoromethyl)pyridin-3-yl)benzamide (55)

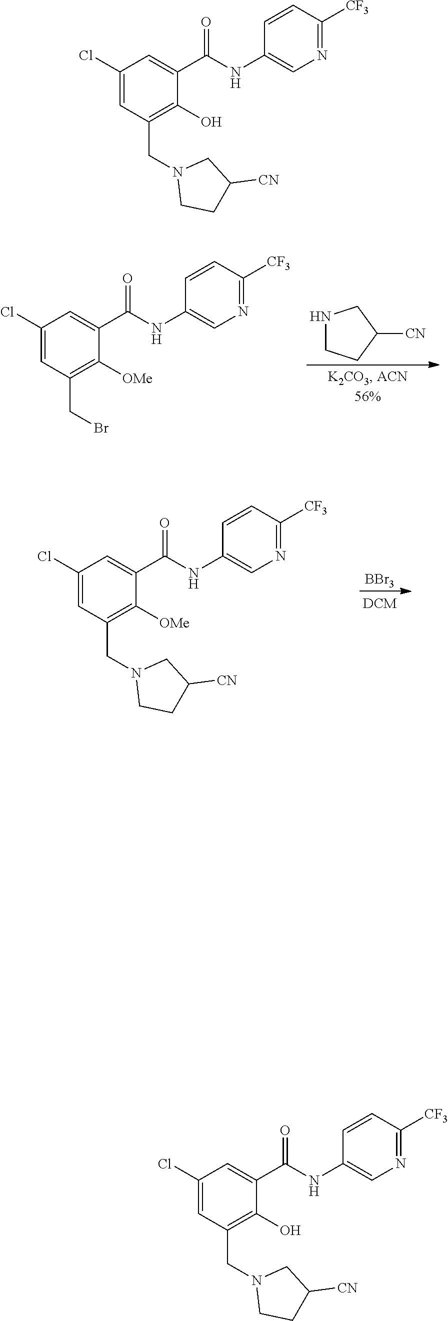 [SMILES: N#CC1CCN(Cc2cc(Cl)cc(C(=O)Nc3ccc(C(F)(F)F)nc3)c2O)C1 ; COc1c(CBr)cc(Cl)cc1C(=O)Nc1ccc(C(F)(F)F)nc1 ; N#CC1CCNC1 ; COc1c(CN2CCC(C#N)C2)cc(Cl)cc1C(=O)Nc1ccc(C(F)(F)F)nc1 ; "BBr3" ; N#CC1CCN(Cc2cc(Cl)cc(C(=O)Nc3ccc(C(F)(F)F)nc3)c2O)C1]

To 3-(bromomethyl)-5-chloro-2-methoxy-N-(6-(trifluoromethyl)pyridin-3-yl)benzamide (500 mg, 1.18 mmol, Example 53) in ACN (10 mL) was added K₂CO₃ (326 mg, 2.36 mmol), and the mixture was stirred at 0° C. for 10 minutes. Pyrrolidine-3-carbonitrile (124 mg, 1.30 mmol, 7.88 μL) was added and the mixture was stirred at 30° C. for 12 h. The reaction mixture was concentrated under reduced pressure to remove solvent. The residue was purified by column chromatography (SiO₂, petroleum ether/Ethyl acetate=1/0 to 1/1) to afford 5-chloro-3-((3-cyanopyrrolidin-1-yl)methyl)-2-methoxy-N-(6-(trifluoromethyl)pyridine-3-yl)benzamide (320 mg, 656.29 μmol, 55.62% yield, 90% purity) as yellow oil.

To 5-chloro-3-((3-cyanopyrrolidin-1-yl)methyl)-2-methoxy-N-(6-(trifluoromethyl)pyridine-3-yl)benzamide (150 mg, 341.82 μmol) in DCM (2 mL), was added BBr₃ (256 mg, 1.03 mmol, 98.81 μL) in DCM (0.5 mL) at −78° C., and the mixture was stirred at 25° C. for 12 h. The reaction mixture was diluted with sat NaHCO₃ (5 mL) and extracted with DCM (5 mL×3). The combined organic layer was washed with brine (5 mL), dried over Na₂SO₄, filtered and concentrated under reduced pressure to give a residue. The residue was purified by prep-HPLC (HCl condition) to afford 5-chloro-3-((3-cyanopyrrolidin-1-yl)methyl)-2-hydroxy-N-(6-(trifluoromethyl)pyridin-3-yl)benzamide.

### Example 56

### 5-chloro-2-hydroxy-N-(6-(trifluoromethyl)benzo[d]thiazol-2-yl)-3-((2-(trifluoromethyl)pyrrolidin-1-yl)methyl)benzamide (56) and 5-chloro-2-hydroxy-N-(6-(trifluoromethyl)benzo[d]thiazol-2-yl)-3-((2-(trifluoromethyl)pyrrolidin-1-yl)methyl)benzamide hydrochloride (56A)

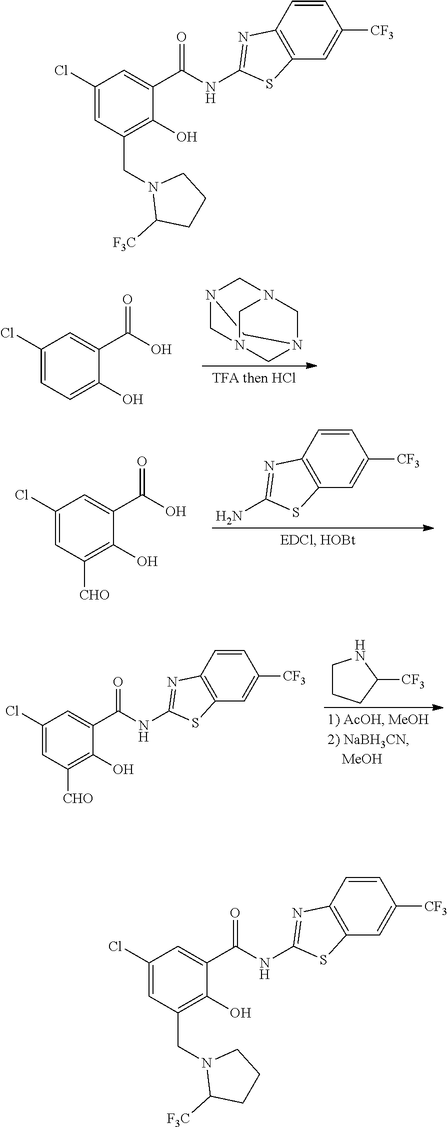

A solution of 5-chloro-2-hydroxybenzoic acid (50 g, 289.74 mmol), and 1,3,5,7-tetraazaadamantane (81.24 g, 579.49 mmol, 108.31 mL) in TFA (500 mL) was stirred at 100° C. for 12 h. The reaction mixture was cooled to 20° C., followed by addition of HCl (500 mL) (3M). After stirring for another 12 h, the solution was concentrated under reduced pressure. The white residue was suspended in water (1000 mL). The solid formed was collected, dried under vacuum to afford compound 5-chloro-3-formyl-2-hydroxybenzoic acid (50 g, crude) as a white solid.

A mixture of 5-chloro-3-formyl-2-hydroxybenzoic acid (3 g, 14.96 mmol), 6-(trifluoromethyl)-benzo[d]thiazol-2-amine (2.6 g, 11.97 mmol) and EDCI (3.4 g, 17.95 mmol), HOBt (3 g, 22.44 mmol) in DCM (150 mL) was heated at 50° C. for 8 h. The reaction mixture was concentrated to remove solvent. Then H₂O (300 mL) was added and NaOH (3M) was added to reach a pH of ~9-10, extracted with ethyl acetate (200 mL×2), then concentrated to remove ethyl acetate. After concentration 200 mL ethyl acetate was added and the precipitated solid was filtered and concentrated under reduced pressure to give 5-chloro-3-formyl-2-hydroxy-N-(6-(trifluoromethyl)benzo[d]thiazol-2-yl)benzamide (1.3 g) as a yellow solid.

To a solution of 2-(trifluoromethyl)pyrrolidine (38 mg, 274.48 gmol), 5-chloro-3-formyl-2-hydroxy-N-(6-(trifluoromethyl)benzo[d]thiazol-2-yl)benzamide (100 mg, 249.53 μmol) in MeOH (5 mL) was added acetic acid (14 mg, 249.53 μmol, 14.27 μL) to pH ~5-6. The mixture was stirred at 30° C. for 12 h then NaBH₃CN (94 mg, 1.50 mmol) was added. The mixture was stirred at 30° C. for another 4 h. The reaction mixture was concentrated under reduced pressure to remove solvent. The residue was purified by prep-HPLC (HCl condition) to afford 5-chloro-2-hydroxy-N-(6-(trifluoromethyl)benzo[d]thiazol-2-yl)-3-((2-(trifluoromethyl)pyrrolidin-1-yl)methyl)benzamide (18 mg, 32.16 μmol, 12.89% yield, 100% purity, HCl) as a white solid. ¹H NMR (400 MHz, METHANOL-d₄) δ ppm 8.30 (s, 1H), 8.04 (d, J=2.45 Hz, 1H), 7.87 (d, J=8.80 Hz, 1H), 7.75 (d, J=8.31 Hz, 1H), 7.49 (d, J=2.45 Hz, 1H), 4.41 (br d, J=14.67 Hz, 1H), 4.04 (d, J=14.18 Hz, 1H), 3.73 (br s, 1H), 3.11-3.25 (m, 1H), 2.65-2.78 (m, 1H), 2.21-2.33 (m, 1H), 2.08 (dt, J=17.24, 3.36 Hz, 1H), 1.97 (br d, J=9.29 Hz, 2H). MS (ESI) [M+H]⁺ requires m/z 524.0, found m/z 524.0.

### Example 57

### 5-chloro-2-hydroxy-N-(6-(trifluoromethyl)benzo[d]thiazol-2-yl)-3-((4-(trifluoromethyl)piperidin-1-yl)methyl)benzamide (57)

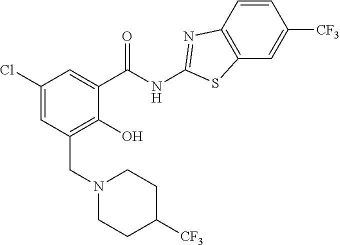 [SMILES: O=C(Nc1nc2ccc(C(F)(F)F)cc2s1)c1cc(Cl)cc(CN2CCC(C(F)(F)F)CC2)c1O]

5-chloro-2-hydroxy-N-(6-(trifluoromethyl)benzo[d]thiazol-2-yl)-3-((4-(trifluoromethyl)piperidin-1-yl)methyl)benzamide was made in a manner similar to Example 56. LCMS: MS (ESI) [M+H]⁺ requires m/z 538.0 found m/z 538.0. ¹H NMR (400 MHz, METHANOL-d₄) δ ppm 8.21-8.34 (m, 2H), 7.75-7.86 (m, 2H), 7.67 (d, J=2.45 Hz, 1H), 4.41 (s, 2H), 3.67 (br d, J=10.76 Hz, 2H), 3.09-3.26 (m, 2H), 2.63 (br s, 1H), 2.18 (br d, J=14.67 Hz, 2H), 1.88 (br d, J=12.72 Hz, 2H)

### Example 58

### 5-chloro-3-((3-fluoropiperidin-1-yl)methyl)-2-hydroxy-N-(6-(trifluoromethyl)benzo[d]thiazol-2-yl)benzamide

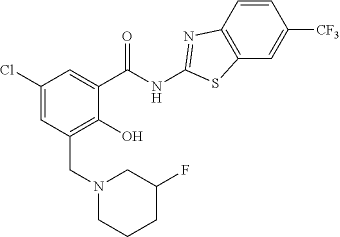 [SMILES: O=C(Nc1nc2ccc(C(F)(F)F)cc2s1)c1cc(Cl)cc(CN2CCCC(F)C2)c1O]

5-chloro-3-((3-fluoropiperidin-1-yl)methyl)-2-hydroxy-N-(6-(trifluoromethyl)benzo[d]thiazol-2-yl)benzamide was made in a similar manner to Example 56. LCMS: MS (ESI) [M+H]⁺ requires m/z 488.07, found m/z 488.1. ¹HNMR (400 MHz, METHANOL-d₄) δ ppm 8.27 (s, 1H), 8.21 (d, J=2.65 Hz, 1H), 7.78 (s, 2H), 7.63 (d, J=2.43 Hz, 1H), 4.97-5.15 (m, 1H), 4.32-4.46 (m, 2H), 3.66 (br s, 1H), 3.37-3.56 (m, 2H), 3.10-3.21 (m, 1H), 2.12 (br s, 2H) 1.87 (br d, J=8.82 Hz, 2H)

### Example 59

### 5-chloro-3-((3,3-difluoropiperidin-1-yl)methyl)-2-hydroxy-N-(6-(trifluoromethyl)benzo[d]thiazol-2-yl)benzamide (59)

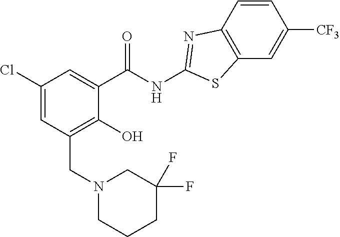 [SMILES: O=C(Nc1nc2ccc(C(F)(F)F)cc2s1)c1cc(Cl)cc(CN2CCCC(F)(F)C2)c1O]

5-chloro-3-((3,3-difluoropiperidin-1-yl)methyl)-2-hydroxy-N-(6-(trifluoromethyl)benzo[d]thiazol-2-yl)benzamide was made in a manner similar to Example 56. LCMS: MS (ESI) [M+H]⁺ requires m/z 506.0, found m/z 506.0. ¹H NMR (400 MHz, DMSO-d₆) δ ppm 8.50 (s, 1H) 7.85-7.92 (m, 2H) 7.77 (br d, J=8.80 Hz, 1H) 7.48 (d, J=2.45 Hz, 1H) 4.10 (br s, 2H) 3.29 (br s, 2H) 2.87 (br s, 2H) 2.05 (br s, 2H) 1.83 (br s, 2H)

### Example 60

### 5-chloro-3-((4-fluoropiperidin-1-yl)methyl)-2-hydroxy-N-(6-(trifluoromethyl)benzo[d]thiazol-2-yl)benzamide (60)

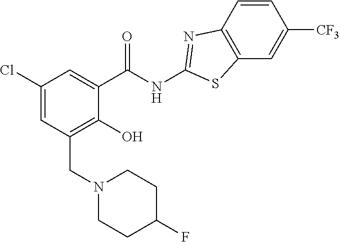 [SMILES: O=C(Nc1nc2ccc(C(F)(F)F)cc2s1)c1cc(Cl)cc(CN2CCC(F)CC2)c1O]

5-chloro-3-((4-fluoropiperidin-1-yl)methyl)-2-hydroxy-N-(6-(trifluoromethyl)benzo[d]thiazol-2-yl)benzamide was made in a manner similar to Example 56. LCMS: MS (ESI) [M+H]⁺ requires m/z 488.0, found m/z 488.1. ¹H NMR (400 MHz, METHANOL-d₄) δ ppm 8.29 (s, 1H) 8.25 (d, J=2.65 Hz, 1H) 7.79 (s, 2H) 7.67 (s, 1H) 5.02 (br s, 1H) 4.42 (s, 2H) 3.35-3.51 (m, 4H) 2.19 (br s, 4H)

### Example 61

### 5-chloro-2-hydroxy-N-(6-(trifluoromethyl)benzo[d]thiazol-2-yl)-3-((3-(trifluoromethyl)piperidin-1-yl)methyl)benzamide (61)

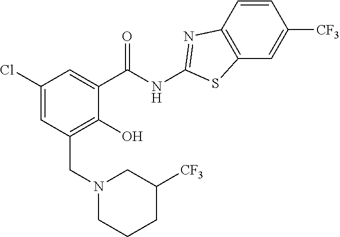 [SMILES: O=C(Nc1nc2ccc(C(F)(F)F)cc2s1)c1cc(Cl)cc(CN2CCCC(C(F)(F)F)C2)c1O]

5-chloro-2-hydroxy-N-(6-(trifluoromethyl)benzo[d]thiazol-2-yl)-3-((3-(trifluoromethyl)piperidin-1-yl)methyl)benzamide was made in a manner similar to Example 56. LCMS: MS (ESI) [M+H]⁺ requires m/z 538.0, found m/z 538.1. ¹H NMR (400 MHz, METHANOL-d₄) δ ppm 8.27 (br s, 1H) 8.22 (br d, J=4.19 Hz, 1H) 7.78 (br s, 2H) 7.65 (d, J=2.65 Hz, 1H) 4.37-4.48 (m, 2H) 3.69 (br d, J=11.25 Hz, 1H) 3.52 (br d, J=12.35 Hz, 1H) 3.17 (t, J=12.24 Hz, 1H) 3.02-3.11 (m, 1H) 2.85 (br s, 1H) 2.03-2.15 (m, 2H) 1.85 (br d, J=12.79 Hz, 1H) 1.57-1.70 (m, 1H)

### Example 62

### 5-chloro-3-((4,4-difluoropiperidin-1-yl)methyl)-2-hydroxy-N-(6-(trifluoromethyl)benzo[d]thiazol-2-yl)benzamide (62)

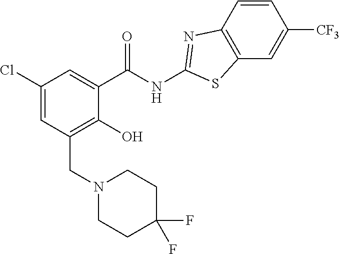 [SMILES: O=C(Nc1nc2ccc(C(F)(F)F)cc2s1)c1cc(Cl)cc(CN2CCC(F)(F)CC2)c1O]

5-chloro-3-((4,4-difluoropiperidin-1-yl)methyl)-2-hydroxy-N-(6-(trifluoromethyl)benzo[d]thiazol-2-yl)benzamide was made in a manner similar to Example 56. LCMS: MS (ESI) [M+H]⁺ requires m/z 506.0, found m/z 506.1. ¹HNMR (400 MHz, METHANOL-d₄) δ ppm 8.29 (s, 1H) 8.27 (d, J=2.87 Hz, 1H) 7.77-7.84 (m, 2H) 7.69 (d, J=2.65 Hz, 1H) 4.47 (s, 2H) 3.47-3.57 (m, 4H) 2.30-2.46 (m, 4H)

### Example 63

### 5-chloro-3-ethyl-2-hydroxy-N-(4-((2-methoxyethoxy)methyl)-6-(trifluoromethyl)benzo[d]thiazol-2-yl)benzamide (63)

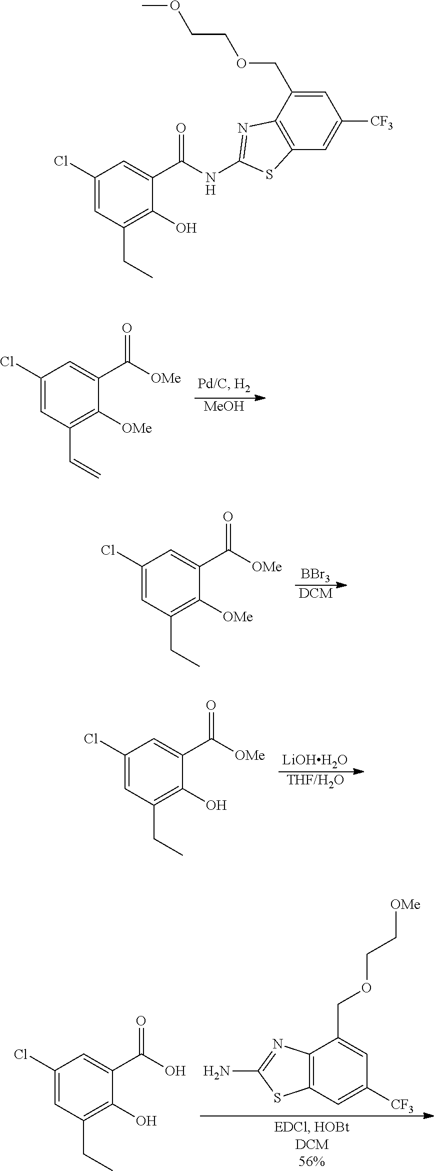

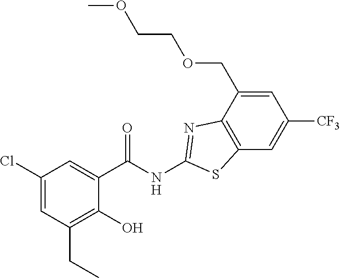 [SMILES: CCc1cc(Cl)cc(C(=O)Nc2nc3c(COCCOC)cc(C(F)(F)F)cc3s2)c1O]

To a solution of methyl 5-chloro-2-methoxy-3-vinylbenzoate (0.45 g, 1.99 mmol) in MeOH (6 mL) was added Pd/C (0.1 g, 10% purity). The mixture was degassed and purged with H₂ 3 times, and then the mixture was stirred at 20° C. for 30 minutes under H₂ (15 Psi) atmosphere. The reaction mixture was filtered and concentrated under reduced pressure to afford methyl 5-chloro-3-ethyl-2-methoxybenzoate (400 mg) as a yellow oil. ¹H NMR (400 MHz, CHLOROFORM-d) δ ppm 7.61-7.65 (m, 1H), 7.34 (d, J=2.65 Hz, 1H), 3.90-3.95 (s, 3H), 3.81-3.85 (s, 3H), 2.63-2.74 (m, 2H), 1.19-1.28 (m, 3H)

To a solution of methyl 5-chloro-3-ethyl-2-methoxybenzoate (0.3 g, 1.31 mmol) in DCM (6 mL) was added dropwise BBr₃ (1.97 g, 7.87 mmol, 758.45 μL) at −78° C., then the resulting mixture was stirred at 40° C. for 12 h. The reaction mixture was quenched by addition sat. NH₄Cl (10 mL), and then extracted with DCM (3 mL×2). The combined organic layer was washed with brine (5 mL), dried over Na₂SO₄, filtered and concentrated under reduced pressure to afford methyl 5-chloro-3-ethyl-2-hydroxybenzoate (0.25 g) as a yellow solid.

To a solution of methyl 5-chloro-3-ethyl-2-hydroxybenzoate (0.25 g, 1.16 mmol) in THF (3 mL) and H₂O (1 mL) was added LiOH·H₂O (146 mg, 3.49 mmol). The mixture was stirred at 25° C. for 12 h. The reaction mixture was extracted with ethyl acetate (5 mL×3), then the aqueous solution was acidized to pH to 5-6 with HCl (1M), and extracted with ethyl acetate (5 mL×3). The combined organic layer was washed with brine (30 mL), dried over Na₂SO₄, filtered and concentrated under reduced pressure to afford 5-chloro-3-ethyl-2-hydroxybenzoic acid (200 mg) as a yellow solid.

To a solution of 5-chloro-3-ethyl-2-hydroxybenzoic acid (80 mg, 398.77 μmol) and 4-((2-methoxyethoxy)methyl)-6-(trifluoromethyl)benzo[d]thiazol-2-amine (122 mg, 398.77 μmol, Example 13) in DCM (3 mL) was added EDCI (91 mg, 478.52 μmol) and HOBt (80 mg, 598.15 μmol). The mixture was stirred at 45° C. for 12 h. The reaction mixture was concentrated under reduced pressure to remove solvent. The residue was purified by prep-HPLC (HCl condition) to afford 5-chloro-3-ethyl-2-hydroxy-N-(4-((2-methoxyethoxy)methyl)-6-(trifluoromethyl)benzo[d]thiazol-2-yl)benzamide (108.9 mg, 222.74 μmol, 55.86% yield, 100% purity) as a white solid. ¹H NMR (400 MHz, DMSO-d₆) δ ppm 8.45 (s, 1H), 8.05 (br s, 1H), 7.80 (s, 1H), 7.44 (d, J=1.96 Hz, 1H), 4.96 (s, 2H), 3.70 (dd, J=5.62, 3.67 Hz, 2H), 3.48-3.60 (m, 2H), 3.28 (s, 3H), 2.65 (q, J=7.83 Hz, 2H), 1.18 (t, J=7.58 Hz, 3H). MS (ESI) [M+H]⁺ requires m/z 489.0, found m/z 489.0

### Example 64

### 5-chloro-3-((cis-2,6-dimethylmorpholino)methyl)-2-hydroxy-N-(4-((2-methoxyethoxy)methyl)-6-(trifluoromethyl)benzo[d]thiazol-2-yl)benzamide (64) and 5-chloro-3-((cis-2,6-dimethylmorpholino)methyl)-2-hydroxy-N-(4-((2-methoxyethoxy)methyl)-6-(trifluoromethyl)benzo[d]thiazol-2-yl)benzamide hydrochloride (64A)

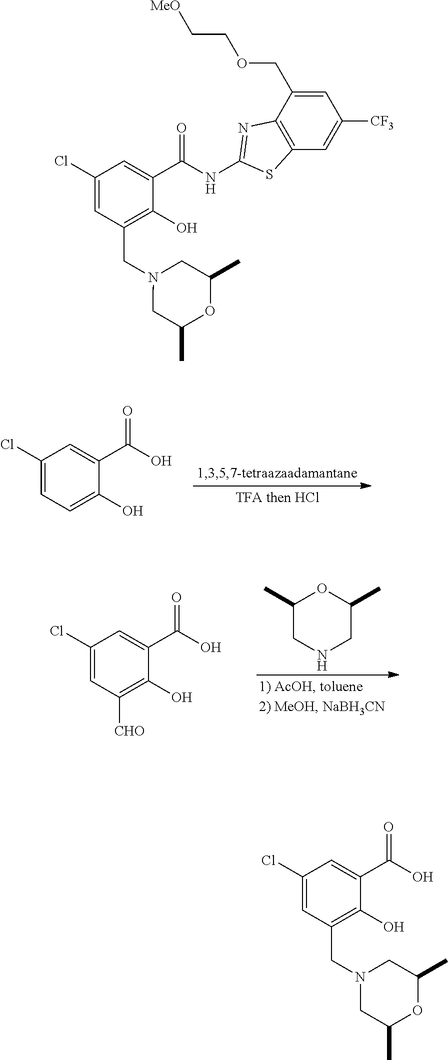

To a solution of 5-chloro-2-hydroxybenzoic acid (50 g, 289.74 mmol) in TFA (500 mL) was added 1,3,5,7-tetraazaadamantane (81.2 g, 579.49 mmol) at 25° C. The mixture was stirred at 100° C. for 12 h, then cooled to 20° C., followed by addition of HCl (500 mL, 3 M). After stirring for another 12 h, the solution was concentrated under reduced pressure. The white residue was suspended in water (1000 mL). The solid was collected, dried under vacuum to afford compound 5-chloro-3-formyl-2-hydroxybenzoic acid (50 g, crude) as a white solid.

To a solution of 5-chloro-3-formyl-2-hydroxybenzoic acid (2 g, 9.97 mmol), and cis-2,6-dimethylmorpholine (2.3 g, 19.94 mmol, 2.46 mL) in toluene (50 mL) was added acetic acid (598 mg, 9.97 mmol, 570.26 μL) to make pH ~5-6. The mixture was stirred at 80° C. for 12 h, then the reaction mixture was concentrated under reduced pressure to remove solvent and then MeOH (50 mL) and NaBH₃CN (3.7 g, 59.83 mmol) were added. The mixture was stirred at 50° C. for another 3 h. The reaction mixture was concentrated under reduced pressure to remove solvent. The residue was purified by prep-HPLC (neutral condition) to afford compound 5-chloro-3-((cis-dimethylmorpholino)methyl]-2-hydroxybenzoic acid (1.73 g, 4.62 mmol, 46.30% yield, 80% purity) as a yellow solid. ¹H NMR (400 MHz, DMSO-d₆) δ ppm 7.67 (d, J=2.93 Hz, 1H), 7.41 (d, J=2.93 Hz, 1H), 3.78-3.92 (m, 2H), 3.21-3.33 (m, 2H), 2.56-2.68 (m, 2H), 1.11 (d, J=6.36 Hz, 6H).

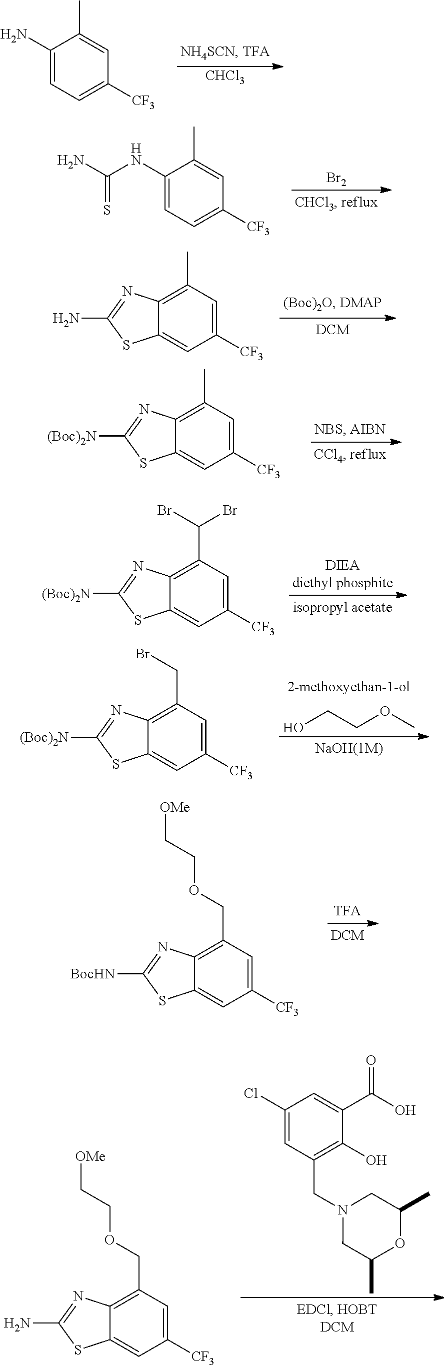

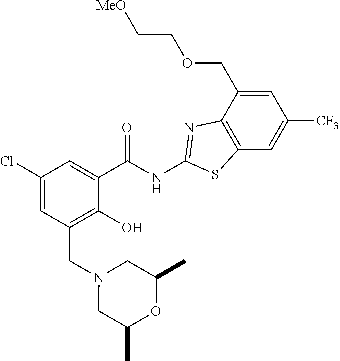 [SMILES: COCCOCc1cc(C(F)(F)F)cc2sc(NC(=O)c3cc(Cl)cc(CN4C[C@@H](C)O[C@@H](C)C4)c3O)nc12]

A mixture of 2-methyl-4-(trifluoromethyl)aniline (22 g, 125.61 mmol), ammonia thiocyanic acid (14.3 g, 188.41 mmol, 14.34 mL) and TFA (35.8 g, 314.02 mmol, 23.25 mL) in CHCl₃ (440 mL) was stirred at 80° C. for 12 h. To the reaction mixture was added water (1000 mL) and extracted with ethyl acetate (500 mL×3). The combined organic layer was washed with brine (600 mL×3), dried over Na₂SO₄, filtered and concentrated under reduced pressure to give a residue. The residue was purified by flash silica gel chromatography (ISCO®; 120 g SepaFlash® Silica Flash Column, Eluent of 5~50% ethyl acetate/petroleum ether gradient @100 mL/min) to afford 1-(2-methyl-4-(trifluoromethyl)phenyl)thiourea (17.2 g, 69.02 mmol, 54.95% yield, 94% purity) as a yellow solid. ¹H NMR (400 MHz, CHLOROFORM-d) δ ppm 8.09 (br s, 1H), 7.50-7.65 (m, 2H), 7.39 (br d, J=7.72 Hz, 1H), 6.12 (br s, 2H), 2.40 (br s, 3H). MS (ESI) [M+H]⁺ requires m/z 235.0, found m/z 235.1

At 0° C., a solution of Br₂ (9.8 g, 61.48 mmol, 3.17 mL) in CHCl₃ (15 mL) was added dropwise to a stirred solution of (2-methyl-4-(trifluoromethyl)phenyl)thiourea (12 g, 51.23 mmol) in CHCl₃ (100 mL), then the mixture was stirred at 70° C. for 12 h, then cooled to 20° C. The precipitated white solid was filtered out and collected, then the solid was diluted with water (500 mL) and neutralized by NH₃·H₂O to pH ~8-9, and extracted with ethyl acetate (250 mL×3). The combined organic layer was washed with brine (500 mL), dried over Na₂SO₄, filtered and concentrated under reduced pressure to give a residue. The residue was purified by column chromatography (SiO₂, petroleum ether/ethyl acetate=1/0 to 5/1) to afford 4-methyl-6-(trifluoromethyl)benzo[d]thiazol-2-amine (91% purity) 13.4 g as a yellow solid. ¹H NMR (400 MHz, CHLOROFORM-d) δ ppm 7.70 (s, 1H), 7.37 (s, 1H), 6.30 (br s, 2H), 2.56 (s, 3H)

To 4-methyl-6-(trifluoromethyl)benzo[d]thiazol-2-amine (13.4 g, 57.70 mmol) and Boc₂O (31.4 g, 144.26 mmol, 33.14 mL) in DCM (400 mL) was added DMAP (704 mg, 5.77 mmol), and the mixture was stirred at 20° C. for 12 h. The reaction mixture was concentrated under reduced pressure to remove solvent. The residue was purified by flash silica gel chromatography (ISCO®;40 g SepaFlash® Silica Flash Column, Eluent of 0~2% ethyl acetate/petroleum ether gradient @70 mL/min) to afford tert-butyl N-tert-butoxycarbonyl-N-(4-methyl-6-(trifluoromethyl)benzo[d]thiazol-2-yl)carbamate (24.76 g, 48.67 mmol, 84.34% yield, 85% purity) as yellow oil. ¹H NMR (400 MHz, CHLOROFORM-d) δ ppm 7.90 (s, 1H), 7.46 (s, 1H), 2.64 (s, 3H), 1.62 (s, 18H)

To tert-butyl N-tert-butoxycarbonyl-N-(4-methyl-6-(trifluoromethyl)benzo[d]thiazol-2-yl)carbamate (20 g, 46.25 mmol) and NBS (24.6 g, 138.74 mmol) in CCl₄ (500 mL) was added AIBN (759 mg, 4.62 mmol), and the mixture was stirred at 80° C. for 12 h. The reaction mixture was concentrated under reduced pressure to remove solvent. The residue was purified by column chromatography (SiO₂, petroleum ether/ethyl acetate=1/0 to 20/1) to afford tert-butyl N-tert-butoxycarbonyl-N-(4-(dibromomethyl)-6-(trifluoromethyl)benzo[d]thiazol-2-yl)carbamate (27 g, 36.32 mmol, 78.53% yield, 79.392% purity) as yellow oil. ¹H NMR (400 MHz, CHLOROFORM-d) δ ppm 8.15 (s, 1H), 8.00-8.05 (m, 1H), 7.42 (s, 1H), 4.13 (q, J=7.34 Hz, 1H), 1.60-1.69 (s, 18H)

Diethyl phosphate (6.3 g, 45.74 mmol, 5.90 mL) was added to a stirred mixture of tert-butyl N-(4-(dibromomethyl)-6-(trifluoromethyl)benzo[d]thiazol-2-yl)-N-tert-butoxycarbonylcarbamate (27 g, 45.74 mmol) in isopropyl acetate (200 mL), N,N-diisopropylethylamine (3.2 g, 25.16 mmol, 4.38 mL) was added dropwise and the mixture was stirred at 20° C. for 12 h. The reaction mixture was diluted with water (500 mL) and extracted with ethyl acetate (200 mL×2). The combined organic layer was washed with brine (500 mL), dried over Na₂SO₄, filtered and concentrated under reduced pressure to give a residue. The residue was purified by column chromatography (SiO₂, Petroleum ether/Ethyl acetate 1/0 to 20/1) to afford t di-tert-butyl (4-(bromomethyl)-6-(trifluoromethyl)benzo[d]thiazol-2-yl)iminodicarbonate as a yellow solid.

To a stirred solution of the di-tert-butyl (4-(bromomethyl)-6-(trifluoromethyl)benzo[d]thiazol-2-yl)iminodicarbonate (5 g, 7.33 mmol) in 2-methoxyethanol (144.7 g, 1.90 mol, 149.95 mL) was added NaOH (1 M, 56.25 mL). The resulting mixture was stirred at 20° C. for 12 h. The reaction mixture was diluted with water (150 mL) and extracted with ethyl acetate (50 mL×2). The combined organic layer was washed with brine (500 mL×2), dried over Na₂SO₄, filtered, and concentrated under reduced pressure to give a residue. The residue was purified by flash silica gel chromatography (ISCO®; 40 g SepaFlash® Silica Flash Column, Eluent of 0~10% ethyl acetate/petroleum ether gradient @70 mL/min) to afford tert-butyl (4-((2-methoxyethoxy)methyl)-6-(trifluoromethyl)benzo[d]thiazol-2-yl]carbamate (2.6 g, 5.30 mmol, 72.30% yield, 82.88% purity) as a white solid.

To a solution of tert-butyl (4-((2-methoxyethoxy)methyl)-6-(trifluoromethyl)benzo[d]thiazol-2-yl]carbamate (0.6 g, 1.48 mmol) in DCM (3 mL) was added TFA (3 mL). The mixture was stirred at 20° C. for 0.5 h. The reaction mixture was concentrated under reduced pressure to remove solvent. The reaction mixture was diluted with water (5 mL) and extracted with DCM (3 mL×3). The combined organic layers were washed with brine (20 mL), dried over Na₂SO₄, filtered and concentrated under reduced pressure to give a residue. The residue was purified by flash silica gel chromatography (ISCO®;12 g SepaFlash® Silica Flash Column, eluent of 0-50% ethyl acetate/petroleum ether gradient @70 mL/min) to afford 4-((2-methoxyethoxy)methyl)-6-(trifluoromethyl)benzo[d]thiazol-2-amine (94.47% purity 1.2 g) as a yellow solid. ¹H NMR (400 MHz, DMSO-d₆) δ ppm 8.04 (s, 1H), 7.98 (br s, 2H), 7.53 (s, 1H), 4.76 (s, 2H), 3.64 (dd, J=5.62, 3.67 Hz, 2H), 3.51 (dd, J=5.62, 4.16 Hz, 2H), 3.24-3.29 (m, 3H)

To a solution of 4-((2-methoxyethoxy)methyl)-6-(trifluoromethyl)benzo[d]thiazol-2-amine (0.5 g, 1.63 mmol), 5-chloro-3-((cis-2,6-dimethylmorpholino)methyl)-2-hydroxybenzoic acid (733 mg, 2.45 mmol) in DCM (30 mL) was added EDCI (375 mg, 1.96 mmol) and HOBt (330 mg, 2.45 mmol). The mixture was stirred at 45° C. for 12 h. The reaction mixture was concentrated under reduced pressure to remove solvent. The residue was purified by prep-HPLC (HCl condition) to afford 5-chloro-3-((cis-2,6-dimethylmorpholino)methyl)-2-hydroxy-N-(4-((2-methoxyethoxy)methyl)-6-(trifluoromethyl)benzo[d]thiazol-2-yl]benzamide (544.77 mg, 836.59 μmol, 51.25% yield, 95.90% purity, HCl) as a yellow solid. ¹H NMR (400 MHz, METHANOL-d₄) δ ppm 8.24 (d, J=2.45 Hz, 1H), 8.20 (s, 1H), 7.79 (s, 1H), 7.71 (d, J=2.45 Hz, 1H), 4.98 (s, 2H), 4.41 (s, 2H), 3.91 (br s, 2H), 3.80 (dd, J=5.62, 3.67 Hz, 2H), 3.65-3.71 (m, 2H), 3.41-3.48 (m, 5H), 2.84 (t, J=11.74 Hz, 2H), 1.25 (d, J=6.36 Hz, 6H). MS (ESI) [M+H]⁺ requires m/z 588.1, found m/z 588.1

### Example 65

### 5-chloro-2-hydroxy-N-(4-((2-methoxyethoxy)methyl)-6-(trifluoromethyl)benzo[d]thiazol-2-yl)-3-(morpholinomethyl)benzamide (65)

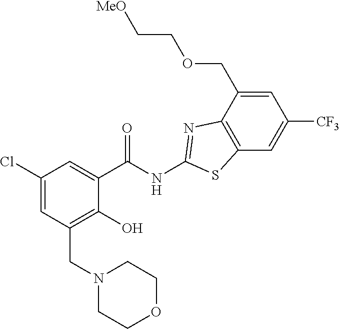 [SMILES: COCCOCc1cc(C(F)(F)F)cc2sc(NC(=O)c3cc(Cl)cc(CN4CCOCC4)c3O)nc12]

5-chloro-2-hydroxy-N-(4-((2-methoxyethoxy)methyl)-6-(trifluoromethyl)benzo[d]thiazol-2-yl)-3-(morpholinomethyl)benzamide was made in a manner similar to Example 64. LCMS: MS (ESI) [M+H]⁺ requires m/z 560.1, found m/z 560.2. ¹H NMR (400 MHz, METHANOL-d₄) δ ppm 8.12-8.19 (m, 2H), 7.74 (s, 1H), 7.68 (d, J=1.96 Hz, 1H), 4.95 (s, 2H), 4.40 (s, 2H), 3.93 (br s, 4H), 3.80 (dd, J=5.38, 3.42 Hz, 2H), 3.64-3.72 (m, 2H), 3.44 (s, 3H), 3.37 (br s, 4H)

### Example 66

### 5-chloro-2-hydroxy-N-(4-((2-methoxyethoxy)methyl)-6-(trifluoromethyl)benzo[d]thiazol-2-yl)-3-(piperidin-1-ylmethyl)benzamide (66)

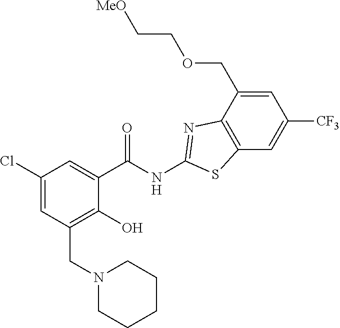 [SMILES: COCCOCc1cc(C(F)(F)F)cc2sc(NC(=O)c3cc(Cl)cc(CN4CCCCC4)c3O)nc12]

5-chloro-2-hydroxy-N-(4-((2-methoxyethoxy)methyl)-6-(trifluoromethyl)benzo[d]thiazol-2-yl)-3-(piperidin-1-ylmethyl)benzamide was made in a manner similar to Example 64. LCMS: MS (ESI) [M+H]⁺ requires m/z 558.1, found m/z 558.2. ¹H NMR (400 MHz, DMSO-d₆) δ ppm 8.40 (s, 1H), 8.01 (br s, 1H), 7.75 (s, 1H) 7.69 (br s, 1H), 4.94 (s, 2H), 4.25 (s, 2H), 3.69 (dd, J=5.14, 3.67 Hz, 2H), 3.42-3.58 (m, 6H), 3.27 (s, 3H), 1.37-1.82 (m, 6H)

### Example 67

### 5-chloro-2-hydroxy-N-(4-methoxy-6-(trifluoromethyl)benzo[d]thiazol-2-yl)-3-methylbenzamide (67)

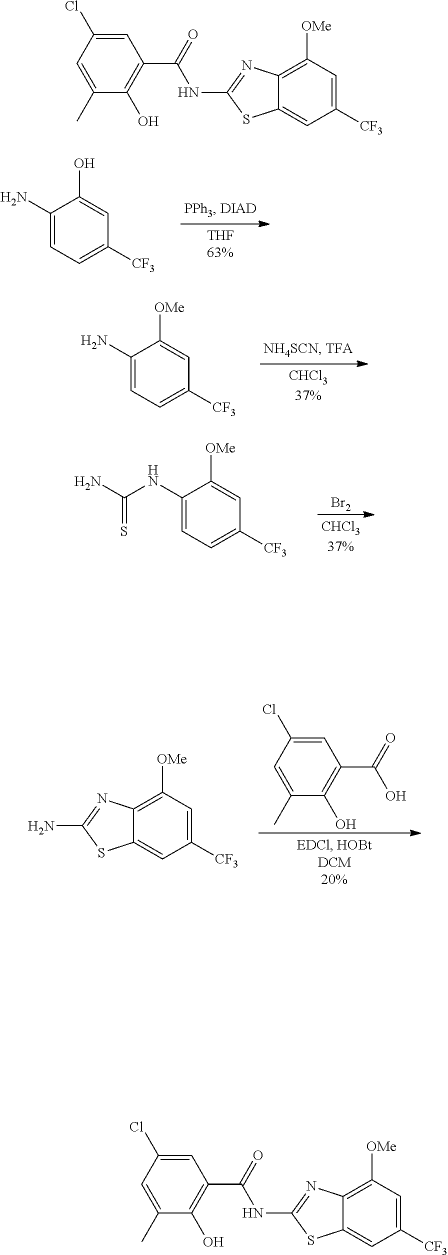

To a solution of 2-amino-5-(trifluoromethyl)phenol (0.5 g, 2.82 mmol) in MeOH (396 mg, 12.36 mmol, 500.34 μL) and THF (10 mL) was added PPh₃ (1.4 g, 5.65 mmol), the mixture was stirred at 20° C. for 5 minutes, then DIAD (1.5 g, 7.62 mmol, 1.48 mL) was added dropwise, and the mixture was stirred at 20° C. for another 12 h. The reaction mixture was concentrated under reduced pressure to remove solvent. The residue was purified by column chromatography (SiO₂, petroleum ether/ethyl acetate=1/0 to 20/1) to afford 2-methoxy-4-(trifluoromethyl)aniline (380 mg, 1.77 mmol, 62.68% yield, 89% purity) as yellow oil.

A mixture of 2-methoxy-4-(trifluoromethyl)aniline (380 mg, 1.99 mmol), ammonium thiocyanate (226 mg, 2.98 mmol, 226.99 μL) and TFA (566 mg, 4.97 mmol, 367.97 μL) in CHCl₃ (8 mL) was stirred at 80° C. for 12 h. To the reaction mixture was added water (5 mL) and extracted with DCM (5 mL×3). The combined organic layer was washed with brine (10 mL×2), dried over Na₂SO₄, filtered and concentrated under reduced pressure to give a residue. The residue was purified by column chromatography (SiO₂, petroleum ether/ethyl acetate=1/0 to 5/1) to afford 1-(2-methoxy-4-(trifluoromethyl)phenyl)thiourea (220 mg, 733.24 μmol, 36.88% yield, 83.40% purity) as a yellow solid. MS (ESI) [M+H]⁺ requires m/z 251.0, found m/z 251.2.

At 0° C., a solution of Br₂ (119 mg, 747.28 μmol, 38.52 μL) in CHCl₃ (1.5 mL) was added dropwise to a stirred solution of 1-(2-methoxy-4-(trifluoromethyl)phenyl)thiourea (170 mg, 679.35 μmol) in CHCl₃ (6 mL), then the mixture was stirred at 70° C. for 12 h. The reaction mixture was concentrated under reduced pressure to remove solvent. The residue was diluted with H₂O (5 mL) and neutralized by NH₃·H₂O to pH ~8-9, and extracted with ethyl acetate (5 mL×3). The combined organic layer was washed with brine (10 mL), dried over Na₂SO₄, filtered and concentrated under reduced pressure to give a residue. The residue was purified by prep-TLC (SiO₂, petroleum ether/ethyl acetate=1:1) to afford 4-methoxy-6-(trifluoromethyl)benzo[d]thiazol-2-amine (62 mg, 238.07 μmol, 35.04% yield, 95.31% purity) as a yellow solid. ¹H NMR (400 MHz, CHLOROFORM-d) δ ppm 7.51 (s, 1H), 7.02 (s, 1H), 5.62 (br s, 2H), 4.02 (s, 3H). MS (ESI) [M+H]⁺ requires m/z 249.0, found m/z 249.0.

To a solution of 4-methoxy-6-(trifluoromethyl)benzo[d]thiazol-2-amine (60 mg, 241.72 μmol) and 5-chloro-2-hydroxy-3-methylbenzoic acid (67 mg, 362.57 μmol) in DCM (5 mL) was added EDCI (55 mg, 290.06 μmol) and HOBt (48 mg, 362.57 μmol). The mixture was stirred at 45° C. for 12 h. The reaction mixture was concentrated under reduced pressure to remove solvent. The residue was purified by prep-HPLC (neutral condition) to afford 5-chloro-2-hydroxy-N-(4-methoxy-6-(trifluoromethyl)benzo[d]thiazol-2-yl)-3-methylbenzamide (20 mg, 49.33 μmol, 20.41% yield, 98.84% purity) as a white solid. ¹H NMR (400 MHz, METHANOL-d₄) δ ppm 7.88-7.90 (m, 1H), 7.81 (s, 1H), 7.30 (d, J=1.96 Hz, 1H), 7.23 (s, 1H), 4.07 (s, 3H), 2.24 (s, 3H). MS (ESI) [M+H]⁺ requires m/z 417.0, found m/z 417.0.

### Example 68

### 5-chloro-2-hydroxy-N-(4-(methoxymethyl)-6-(trifluoromethyl)benzo[d]thiazol-2-yl)-3-methylbenzamide (68)

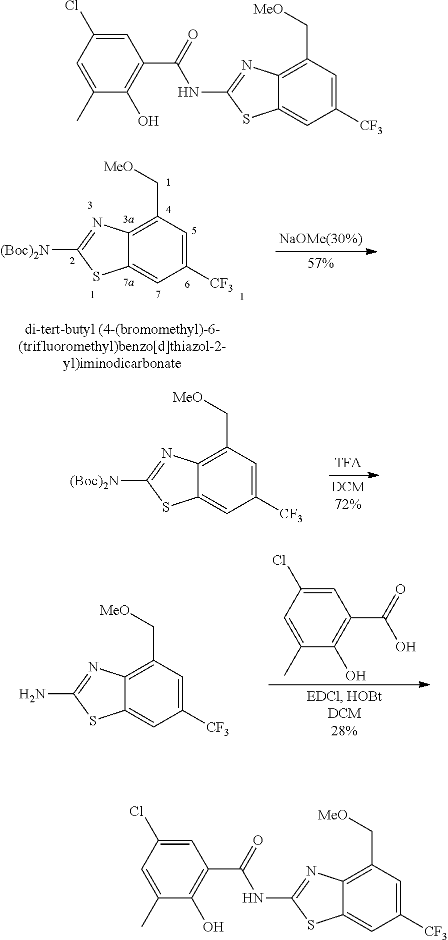

di-tert-butyl (4-(bromomethyl)-6-(trifluoromethyl)benzo[d]thiazol-2-yl)iminodicarbonate (0.5 g, 1.22 mmol, Example 64) was added to NaOMe (10 mL) (30%). The mixture was stirred at 30° C. for 12 h. The reaction mixture was diluted with water (20 mL) and extracted with ethyl acetate (5 mL×2). The combined organic layer was washed with brine (30 mL), dried over Na₂SO₄, filtered and concentrated under reduced pressure to give a residue. The residue was purified by flash silica gel chromatography (ISCO®; 4 g SepaFlash® Silica Flash Column, Eluent of 0~7% ethyl acetate/petroleum ether gradient @70 mL/min) to afford tert-butyl (tert-butoxycarbonyl)(4-(methoxymethyl)-6-(trifluoromethyl)benzo[d]thiazol-2-yl)carbamate (280 mg, 688.37 μmol, 56.62% yield, 89.08% purity) as a white solid.

To a solution of tert-butyl N-[4-(methoxymethyl)-6-(trifluoromethyl)-1,3-benzothiazol-2-yl]carbamate (280 mg, 772.70 μmol) in DCM (3 mL) was added TFA (1 mL). The mixture was stirred at 20° C. for 2 h. The reaction mixture was concentrated under reduced pressure to remove solvent. The residue was diluted with water (3 mL) and extracted with ethyl acetate (3 mL×2). The combined organic layer was washed with brine (4 mL×2), dried over Na₂SO₄, filtered and concentrated under reduced pressure to give a residue. The residue was purified by flash silica gel chromatography (ISCO®; 4 g SepaFlash® Silica Flash Column, Eluent of 0~50% ethyl acetate/petroleum ether gradient @70 mL/min) to afford 4-(methoxymethyl)-6-(trifluoromethyl)benzo[d]thiazol-2-amine (180 mg, 558.74 μmol, 72.31% yield, 81.40% purity) as a yellow solid.

To a solution of 4-(methoxymethyl)-6-(trifluoromethyl)benzo[d]thiazol-2-amine (130 mg, 495.71 μmol), and 5-chloro-2-hydroxy-3-methylbenzoic acid (138 mg, 743.56 μmol) in DCM (6 mL), was added EDCI (114 mg, 594.85 μmol) and HOBt (100 mg, 743.56 μmol). The mixture was stirred at 45° C. for 12 h. The reaction mixture was concentrated under reduced pressure to remove solvent. The residue was purified by prep-HPLC (neutral condition) to afford 5-chloro-2-hydroxy-N-(4-(methoxymethyl)-6-(trifluoromethyl)benzo[d]thiazol-2-yl)-3-methylbenzamide (63.3 mg, 139.82 μmol, 28.21% yield, 95.16% purity) as a yellow solid. ¹H NMR (400 MHz, DMSO-d₆) δ ppm 8.43 (br s, 1H), 8.00 (br s, 1H) 7.72 (br s, 1H), 7.44 (br s, 1H), 4.85 (br s, 2H), 3.41 (br s, 3H), 2.21 (br s, 3H). MS (ESI) [M+H]⁺ requires m/z 431.0, found m/z 431.1.

### Example 69

### 3-((bis(2-methoxyethyl)amino)methyl)-5-chloro-2-hydroxy-N-(4-(methoxymethyl)-6-(trifluoromethyl)benzo[d]thiazol-2-yl)benzamide (69)

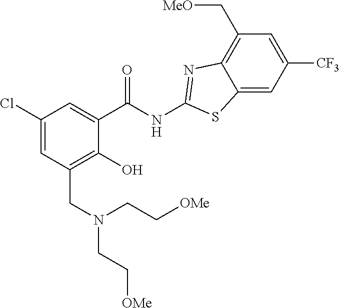 [SMILES: COCCN(CCOC)Cc1cc(Cl)cc(C(=O)Nc2nc3c(COC)cc(C(F)(F)F)cc3s2)c1O]

3-((bis(2-methoxyethyl)amino)methyl)-5-chloro-2-hydroxy-N-(4-(methoxymethyl)-6-(trifluoromethyl)benzo[d]thiazol-2-yl)benzamide was made in a manner similar to Example 68. LCMS: MS (ESI) [M+H]⁺ requires m/z 562.1, found m/z 562.2. ¹H NMR (400 MHz, METHANOL-d₄) δ ppm 7.99 (s, 2H), 7.55 (s, 1H), 7.44 (d, J=2.21 Hz, 1H), 4.68 (s, 2H), 4.35 (s, 2H), 3.55-3.62 (m, 4H), 3.28-3.31 (m, 5H), 3.23 (s, 6H), 3.12 (dt, J=,3.14, 1.63 Hz, 2H).

### Example 70

### 3-((bis(2-methoxyethyl)amino)methyl)-5-chloro-2-hydroxy-N-(4-((2-methoxyethoxy)methyl)-6-(trifluoromethyl)benzo[d]thiazol-2-yl)benzamide (70)

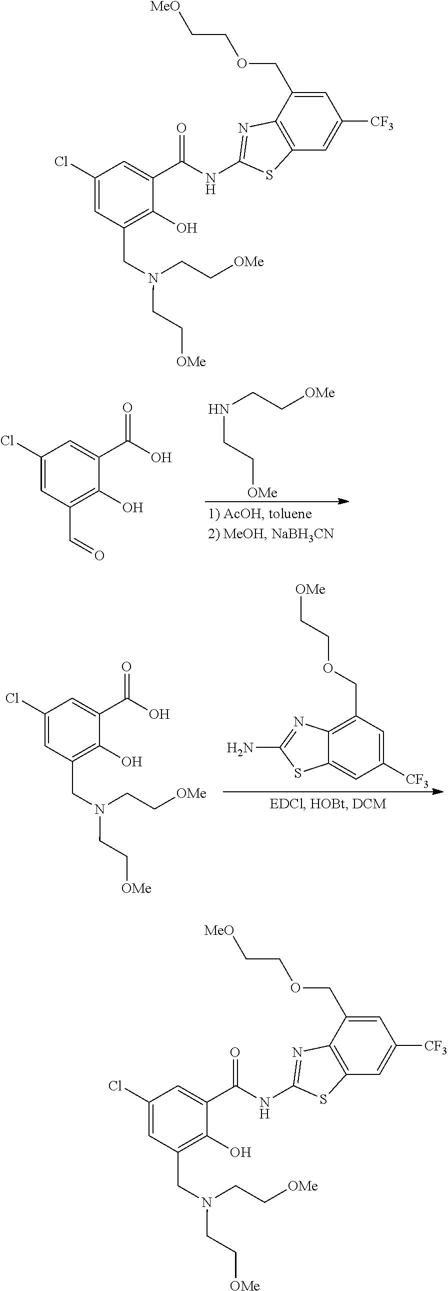 [SMILES: COCCOCc1cc(C(F)(F)F)cc2sc(NC(=O)c3cc(Cl)cc(CN(CCOC)CCOC)c3O)nc12 ; O=Cc1cc(Cl)cc(C(=O)O)c1O ; COCCNCCOC ; COCCN(CCOC)Cc1cc(Cl)cc(C(=O)O)c1O ; COCCOCc1cc(C(F)(F)F)cc2sc(N)nc12 ; COCCOCc1cc(C(F)(F)F)cc2sc(NC(=O)c3cc(Cl)cc(CN(CCOC)CCOC)c3O)nc12]

To a solution of 5-chloro-3-formyl-2-hydroxybenzoic acid (0.5 g, 2.49 mmol) and 2-methoxy-N-(2-methoxyethyl)ethanamine (664 mg, 4.99 mmol, 736.17 μL) in toluene (10 mL) was added acetic acid (149 mg, 2.49 mmol, 142.57 μL) to pH ~5-6. The mixture was stirred at 80° C. for 12 h. The reaction mixture was concentrated under reduced pressure to remove solvent and then MeOH (10 mL) and NaBH₃CN (939 mg, 14.96 mmol) were added. The mixture was stirred at 50° C. for another 3 h. The reaction mixture was concentrated under reduced pressure to remove solvent. The residue was purified by prep-HPLC (neutral conditions) to afford 3-((bis(2-methoxyethyl)amino)methyl)-5-chloro-2-hydroxybenzoic acid (400 mg, 352.46 μmol, 14.14% yield, 28% purity) as a white solid. ¹H NMR (400 MHz, DMSO-d₆) δ ppm 7.63 (d, J=2.93 Hz, 1H), 7.38 (d, J=2.93 Hz, 1H), 4.34 (s, 2H), 3.67 (t, J=4.89 Hz, 4H), 3.31 (br t, J=4.65 Hz, 4H), 3.27 (s, 6H).

To a solution of 3-((bis(2-methoxyethyl)amino)methyl)-5-chloro-2-hydroxybenzoic acid (155 mg, 489.71 μmol), 4-((2-methoxyethoxy)methyl)-6-(trifluoromethyl)benzo[d]thiazol-2-amine (100 mg, 326.47 μmol, Example 13) in DCM (5 mL) was added EDCI (75 mg, 391.77 μmol) and HOBt (66 mg, 489.71 μmol). The mixture was stirred at 50° C. for 12 h. The reaction mixture was concentrated under reduced pressure to remove solvent. The residue was purified by prep-HPLC (HCl condition) to afford 3-((bis(2-methoxyethyl)amino)methyl)-5-chloro-2-hydroxy-N-(4-((2-methoxyethoxy)methyl)-6-(trifluoromethyl)benzo[d]thiazol-2-yl]benzamide (79.6 mg, 122.63 μmol, 37.56% yield, 98.98% purity, HCl) as a white solid. ¹H NMR (400 MHz, METHANOL-d₄) δ ppm 8.14 (d, J=2.45 Hz, 1H), 8.10 (s, 1H), 7.70 (s, 1H), 7.56 (d, J=2.45 Hz, 1H), 4.88 (s, 2H), 4.46 (s, 2H), 3.64-3.72 (m, 6H), 3.53-3.60 (m, 2H), 3.36-3.44 (m, 4H), 3.33 (s, 3H), 3.20 (dt, J=3.30, 1.53 Hz, 6H). MS (ESI) [M+H]⁺ requires m/z 606.1, found m/z 606.2.

### Example 71

### 5-chloro-2-hydroxy-3-methyl-N-(4-(tetrahydrofuran-2-yl)-6-(trifluoromethyl)benzo[d]thiazol-2-yl)benzamide (71)

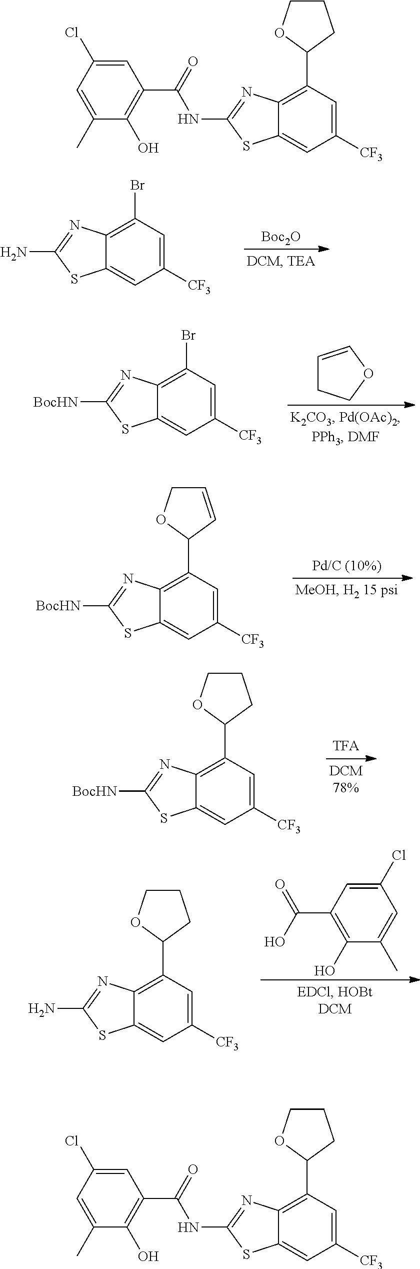

To a solution of 4-bromo-6-(trifluoromethyl)benzo[d]thiazol-2-amine (2.6 g, 8.75 mmol) in DCM (60 mL) was added TEA (2.6 g, 26.25 mmol, 3.65 mL) and Boc₂O (4.7 g, 21.88 mmol, 5.03 mL) and DMAP (53 mg, 437.57 μmol). The mixture was stirred at 20° C. for 12 h. The reaction mixture was concentrated under reduced pressure to remove solvent. The residue was purified by column chromatography (SiO₂, petroleum ether/ethyl acetate=1/0 to 100/1) to afford tert-butyl (4-bromo-6-(trifluoromethyl)benzo[d]thiazol-2-yl)carbamate (1.5 g, 1.77 mmol, 20.28% yield, 47% purity) as a white solid.

A mixture of tert-butyl (4-bromo-6-(trifluoromethyl)benzo[d]thiazol-2-yl)carbamate (1 g, 2.52 mmol), 2,3-dihydrofuran (352 mg, 5.04 mmol, 380.70 μL), K₂CO₃ (1 g, 7.55 mmol), diacetoxypalladium (56 mg, 251.76 μmol) and PPh₃ (132 mg, 503.51 μmol) in DMF (10 mL) was degassed and purged with N₂ 3 times, then the mixture was stirred at 110° C. for 2 h under N₂ atmosphere. The reaction mixture was diluted with water (100 mL) and extracted with ethyl acetate (40 mL×3). The combined organic layer was washed with brine (100 mL×3), dried over Na₂SO₄, filtered and concentrated under reduced pressure to give a residue. The residue was purified by flash silica gel chromatography (ISCO®;4 g SepaFlash® Silica Flash Column, eluent of 0~20% ethyl acetate/petroleum ether gradient @70 mL/min) to afford tert-butyl (4-(2,5-dihydrofuran-2-yl)-6-(trifluoromethyl)benzo[d]thiazol-2-yl)carbamate (254 mg, 657.37 μmol, 26.11% yield) as yellow oil.

A mixture of tert-butyl (4-(2,5-dihydrofuran-2-yl)-6-(trifluoromethyl)benzo[d]thiazol-2-yl)carbamate (0.2 g, 517.61 μmol), Pd/C (10 mg, 517.61 μmol, 10% purity), and NH₃·H₂O (145 mg, 1.04 mmol, 159.49 μL, 25% purity) in MeOH (4 mL), was degassed and purged with H₂ 3 times, then the mixture was stirred at 20° C. for 5 min under H₂ (15 psi) atmosphere. The reaction mixture was filtered and concentrated under reduced pressure to afford tert-butyl (4-tetrahydrofuran-2-yl)-6-(trifluoromethyl)benzo[d]thiazol-2-yl)carbamate (120 mg) as a white solid.

To a solution of tert-butyl (4-tetrahydrofuran-2-yl)-6-(trifluoromethyl)benzo[d]thiazol-2-yl)carbamate (120 mg, 308.96 μmol) in DCM (2 mL) was added TFA (2 mL). The mixture was stirred at 20° C. for 1 h. The reaction mixture was concentrated under reduced pressure to remove solvent. The residue was purified by column chromatography (SiO₂, petroleum ether/ethyl acetate=1/0 to 3/1) to afford 4-(tetrahydrofuran-2-yl)-6-(trifluoromethyl)benzo[d]thiazol-2-amine (80 mg, 241.62 μmol, 78.21% yield, 87.071% purity) as a yellow solid.

To a solution of 4-(tetrahydrofuran-2-yl)-6-(trifluoromethyl)benzo[d]thiazol-2-amine (60 mg, 208.12 μmol), 5-chloro-2-hydroxy-3-methylbenzoic acid (42 mg, 228.94 μmol) in DCM (5 mL) was added EDCI (47 mg, 249.75 μmol) and HOBt (42 mg, 312.19 μmol). The mixture was stirred at 45° C. for 12 h. The reaction mixture was concentrated under reduced pressure to remove solvent. The residue was purified by prep-HPLC (neutral condition) to afford 5-chloro-2-hydroxy-3-methyl-N-(4-(tetrahydrofuran-2-yl)-6-(trifluoromethyl)benzo[d]thiazol-2-yl)benzamide (6.06 mg, 12.84 μmol, 6.17% yield, 96.78% purity) was obtained as a white solid. ¹H NMR (400 MHz, METHANOL-d₄) δ ppm 8.10 (s, 1H), 7.85 (br s, 1H), 7.70 (br s, 1H), 7.26 (br s, 1H), 5.60 (br s, 1H), 4.23 (br d, J=7.83 Hz, 1H), 4.02 (br d, J=7.34 Hz, 1H), 2.65 (br s, 1H), 2.24 (s, 3H), 2.07 (br d, J=6.85 Hz, 2H), 1.81 (br s, 1H). MS (ESI) [M+H]⁺ requires m/z 457.0, found m/z 457.1.

### Example 72

### 5-chloro-3-((cis-2,6-dimethylmorpholino)methyl)-2-hydroxy-N-(4-(methylsulfonamidomethyl)-6-(trifluoromethyl)benzo[d]thiazol-2-yl)benzamide (72)

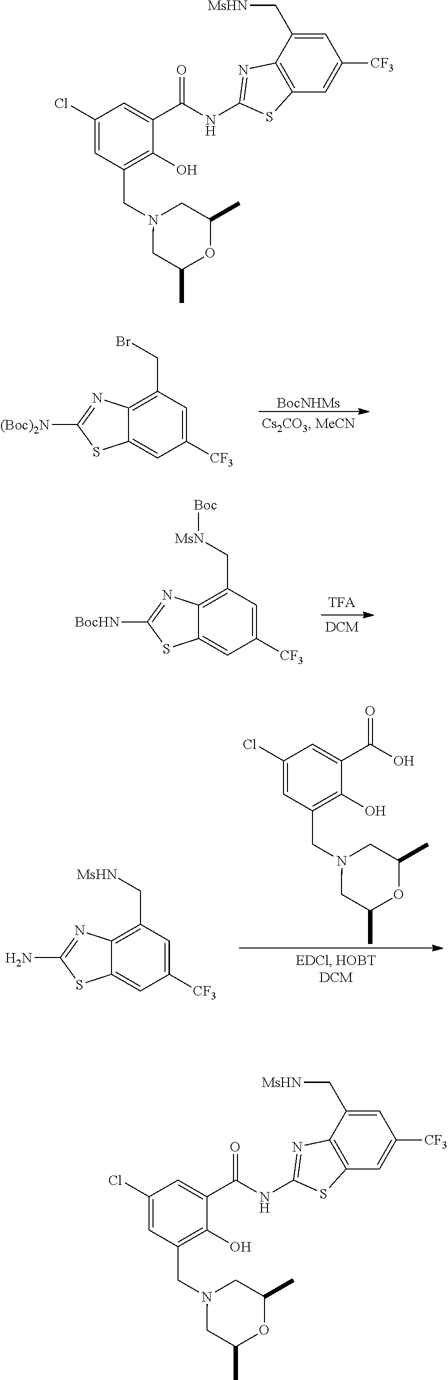

To a solution of tert-butyl(methylsulfonyl)carbamate (381 mg, 1.96 mmol) in MeCN (30 mL) was added Cs₂CO₃ (1.9 g, 5.87 mmol). The mixture was stirred at 80° C. for 3 h, then tert-butyl N-(4-(bromomethyl)-6-(trifluoromethyl)benzo[d]thiazol-2-yl)-N-tert-butoxycarbonylcarbamate (1 g, 1.96 mmol, Example 64) was added, the mixture was stirred at 80° C. for 12 h. The reaction mixture was concentrated under reduced pressure to remove solvent. The residue was diluted with water (5 mL) and extracted with ethyl acetate (7 mL×3). The combined organic layer was washed with brine (20 mL), dried over Na₂SO₄, filtered and concentrated under reduced pressure to give a residue. The residue was purified by flash silica gel chromatography (ISCO®, 12 g SepaFlash® Silica Flash Column, Eluent of 0-10% ethylacetate/petroleum ether gradient @70 mL/min) to afford tert-butyl ((2-((tert-butoxycarbonyl)amino)-6-(trifluoromethyl)benzo[d]thiazol-4-yl)methyl)(methylsulfonyl)carbamate (0.3 g, 481.77 μmol, 24.6% yield, 84.4% purity) as yellow oil.

To a solution of tert-butyl ((2-((tert-butoxycarbonyl)amino)-6-(trifluoromethyl)benzo[d]thiazol-4-yl)methyl)(methylsulfonyl)carbamate (0.3 g, 570.82 μmol) in DCM (3.0 mL) was added TFA (1.0 mL). The mixture was stirred at 20° C. for 2 h. The mixture was adjusted to pH 7-8 with sat. NaHCO₃, the residue was diluted with water (10 mL) and extracted with DCM (5 mL×3). The combined organic layers were washed with brine (10 mL), dried over Na₂SO₄, filtered and concentrated under reduced pressure to give a residue. The residue was purified by flash silica gel chromatography (ISCO®, 4 g SepaFlash® Silica Flash Column, Eluent of 0~50% ethyl acetate/petroleum ether gradient @70 mL/min) to afford N-((2-amino-6-(trifluoromethyl)benzo[d]thiazol-4-yl)methyl)methanesulfonamide (90 mg, 190.88 μmol, 33.4% yield, 69% purity) as a white solid.

To a solution of N-((2-amino-6-(trifluoromethyl)benzo[d]thiazol-4-yl)methyl)methanesulfonamide (30 mg, 92.21 μmol), 5-chloro-3-((cis-2,6-dimethylmorpholino)methyl)-2-hydroxybenzoic acid (30 mg, 101.44 μmol, Example 64) in DCM (4.0 mL) was added EDCI (22 mg, 119.88 μmol) and HOBt (18 mg, 138.32 μmol). The mixture was stirred at 45° C. for 12 h. The reaction mixture was concentrated under reduced pressure to remove solvent. The residue was purified by prep-HPLC (HCl condition) to afford 5-chloro-3-((cis-2,6-dimethylmorpholino)methyl)-2-hydroxy-N-(4-(methanesulfonamidomethyl)-6-(trifluoromethyl)benzo[d]thiazol-2-yl)benzamide (44.18 mg, 99.57% purity, HCl) as a white solid. ¹H NMR (400 MHz, METHANOL-d₄) δ ppm 8.26-8.32 (m, 2H), 7.84 (s, 1H), 7.75 (s, 1H), 4.77 (s,2H), 4.42 (s, 2H), 3.90 (br s, 2H), 3.44 (br d, J=12.13 Hz, 2H), 3.00 (s, 3H), 2.84 (t, J=11.69 Hz, 2H), 1.25 (d, J=6.17 Hz, 6H); MS (ESI) [M+H]⁺ requires m/z 607.10, found m/z 607.1.

### Example 73

### 5-chloro-2-hydroxy-3-methyl-N-(4-(methylsulfonamidomethyl)-6-(trifluoromethyl)benzo[d]thiazol-2-yl)benzamide (73)

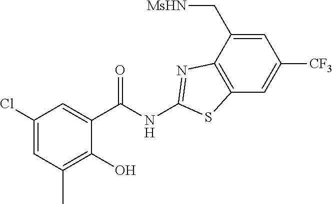

5-chloro-2-hydroxy-3-methyl-N-(4-(methylsulfonamidomethyl)-6-(trifluoromethyl)benzo[d]thiazol-2-yl)benzamide was made in a manner similar to Example 72. LCMS: MS (ESI) [M+H]⁺ requires m/z 494.01, found m/z 493.9. ¹HNMR (400 MHz, DMSO-d₆) δ ppm 8.47 (s, 1H), 8.05 (br s, 1H), 7.83 (s, 1H), 7.77 (t, J=6.17 Hz, 1H), 7.50 (br s, 1H), 4.67 (br d, J=5.95 Hz, 2H), 2.98 (s, 3H) 2.23 (s, 3H)

### Example 74

### 3-((bis(2-methoxyethyl)amino)methyl)-5-chloro-2-hydroxy-N-(4-(methylsulfonamidomethyl)-6-(trifluoromethyl)benzo[d]thiazol-2-yl)benzamide (74)

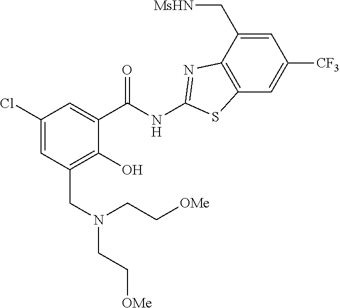

3-((bis(2-methoxyethyl)amino)methyl)-5-chloro-2-hydroxy-N-(4-(methylsulfonamidomethyl)-6-(trifluoromethyl)benzo[d]thiazol-2-yl)benzamide was made in a manner similar to Example 72. LCMS: MS (ESI) [M+H]⁺ requires m/z 625.11, found m/z 625.1. ¹H NMR (400 MHz, METHANOL-d₄) δ ppm 8.26 (s, 2H), 7.83 (s, 1H), 7.71 (d, J=2.21 Hz, 1H), 4.75 (s, 2H), 4.57 (s, 2H), 3.75-3.80 (m, 4H), 3.47-3.52 (m, 4H), 3.42 (s, 6H), 2.99 (s, 3H)

### Example 75

### 5-chloro-2-hydroxy-3-methyl-N-(4-((2-(4-methylpiperazin-1-yl)ethoxy)methyl)-6-(trifluoromethyl)benzo[d]thiazol-2-yl)benzamide (75)

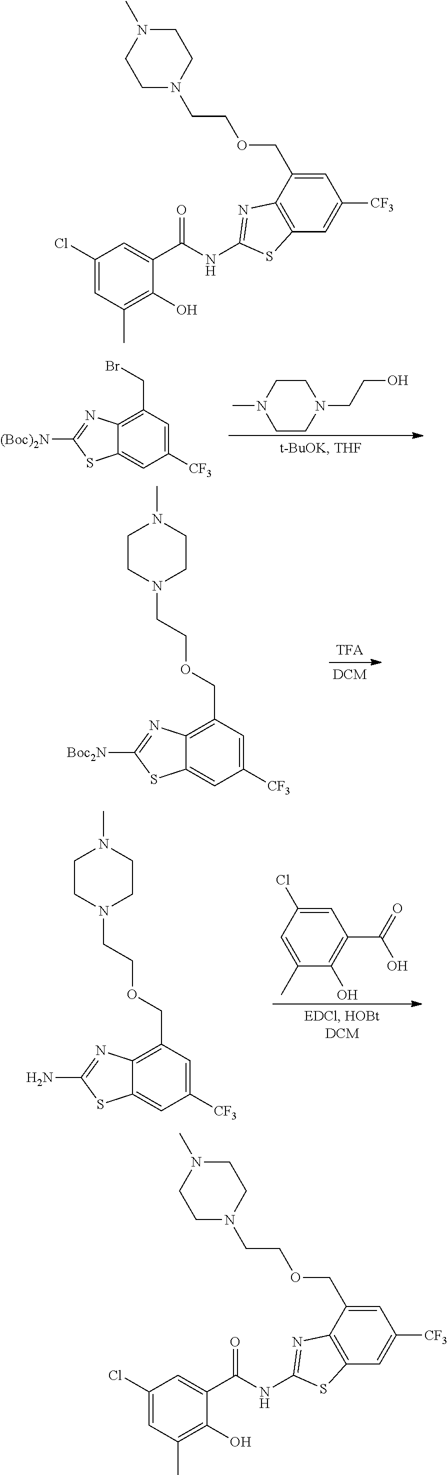

To a mixture of tert-butyl N-(4-(bromomethyl)-6-(trifluoromethyl)benzo[d]thiazol-2-yl)-N-tert-butoxycarbonylcarbamate (0.1 g, 195.56 μmol, Example 64), 2-(4-methylpiperazin-1-yl)ethan-1-ol (56 mg, 391.12 μmol) in THF (4.0 mL) was added t-BuOK (1 M, 782.24 μL, in THF) at 0° C., then the mixture was stirred at 20° C. for 1 h. The residue was diluted with water (3 mL) and extracted with ethyl acetate (3 mL×3). The combined organic layers were washed with brine (5 mL), dried over Na₂SO₄, filtered and concentrated under reduced pressure to give a residue. The residue was purified by prep-TLC (SiO₂, DCM/MeOH=10/1) to afford tert-butyl N-tert-butoxycarbonyl-N-(4-(2-(4-methylpiperazin-1-yl)ethoxymethyl)-6-(trifluoromethyl)benzo[d]thiazol-2-yl)carbamate (100 mg, 104.93 μmol, 53.6% yield, 60.3% purity) as yellow oil.

To a solution of tert-butyl N-tert-butoxycarbonyl-N-(4-(2-(4-methylpiperazin-1-yl)ethoxymethyl)-6-(trifluoromethyl)benzo[d]thiazol-2-yl)carbamate (0.1 g, 174.02 μmol) in DCM (2.0 mL) was added TFA (1 mL). The mixture was stirred at 20° C. for 1 h. The reaction mixture was pulled into sat. NaHCO₃ (10 mL), and then extracted with DCM (3 mL×4). The combined organic layer was washed with brine (10 mL×2), dried over Na₂SO₄, filtered and concentrated under reduced pressure to give 4-((2-(4-methylpiperazin-1-yl)ethoxy)methyl)-6-(trifluoromethyl)benzo[d]thiazol-2-amine (50 mg, crude) as yellow oil.

A mixture of 4-((2-(4-methylpiperazin-1-yl)ethoxy)methyl)-6-(trifluoromethyl)benzo[d]thiazol-2-amine (25 mg, 66.77 μmol), 5-chloro-2-hydroxy-3-methylbenzoic acid (14 mg, 80.12 μmol), EDCI (15 mg, 80.12 μmol), HOBt (13 mg, 100.15 μmol) in DCM (3.0 mL), was stirred at 45° C. for 12 h. The reaction mixture was concentrated under reduced pressure to remove solvent. The residue was purified by prep-HPLC (HCL condition) to afford 5-chloro-2-hydroxy-3-methyl-N-(4-((2-(4-methylpiperazin-1-yl)ethoxy)methyl)-6-(trifluoromethyl)benzo[d]thiazol-2-yl)benzamide (19.1 mg, 99.49% purity, HCl salt) as a white solid. ¹H NMR (400 MHz, DMSO-d₆) δ ppm 8.51 (br s, 1H), 8.03 (br s, 1H), 7.86 (s, 1H), 7.49 (br s, 1H), 4.99 (s, 2H), 3.98 (br s, 2H), 3.24-3.43 (m, 10H), 2.80 (br s, 3H), 2.24 (s, 3H); MS (ESI) [M+H]⁺ requires m/z 543.14, found m/z 543.1

### Example 76

### 5-chloro-3-((cis-2,6-dimethylmorpholino)methyl)-2-hydroxy-N-(4-((2-(4-methylpiperazin-1-yl)ethoxy)methyl)-6-(trifluoromethyl)benzo[d]thiazol-2-yl)benzamide (76)

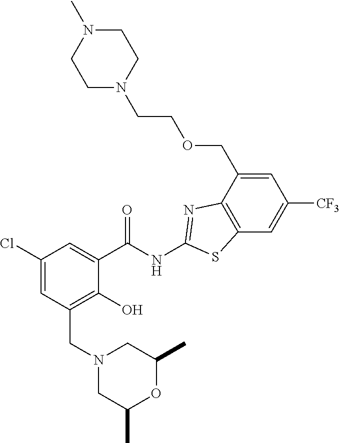 [SMILES: C[C@@H]1CN(Cc2cc(Cl)cc(C(=O)Nc3nc4c(COCCN5CCN(C)CC5)cc(C(F)(F)F)cc4s3)c2O)C[C@H](C)O1]

5-chloro-3-((cis-2,6-dimethylmorpholino)methyl)-2-hydroxy-N-(4-((2-(4-methylpiperazin-1-yl)ethoxy)methyl)-6-(trifluoromethyl)benzo[d]thiazol-2-yl)benzamide was made in a manner similar to Example 75. LCMS: MS (ESI) [M+H]⁺ requires m/z 656.22, found m/z 656.3. ¹H NMR (400 MHz, METHANOL-d₄) δ ppm 8.28-8.35 (m, 2H), 7.87 (s, 1H), 7.79 (d, J=2.43 Hz, 1H), 5.09 (s, 2H), 4.45 (s, 2H), 4.06-4.12 (m, 2H), 3.54-4.05 (m, 12H), 3.47 (br d, J=11.91 Hz, 2H), 3.00 (s, 3H), 2.87 (t, J=11.80 Hz, 2H), 1.24 (d, J=6.17 Hz, 6H)

### Example 77

### 3-((bis(2-methoxyethyl)amino)methyl)-5-chloro-2-hydroxy-N-(4-((2-(4-methylpiperazin-1-yl)ethoxy)methyl)-6-(trifluoromethyl)benzo[d]thiazol-2-yl)benzamide (77)

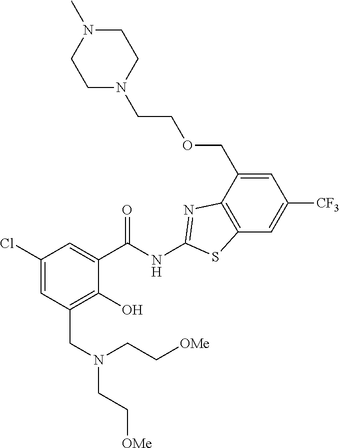 [SMILES: COCCN(CCOC)Cc1cc(Cl)cc(C(=O)Nc2nc3c(COCCN4CCN(C)CC4)cc(C(F)(F)F)cc3s2)c1O]

3-((bis(2-methoxyethyl)amino)methyl)-5-chloro-2-hydroxy-N-(4-((2-(4-methylpiperazin-1-yl)ethoxy)methyl)-6-(trifluoromethyl)benzo[d]thiazol-2-yl)benzamide was made in a manner similar to Example 75. LCMS: MS (ESI) [M+H]⁺ requires m/z 674.23, found m/z 674.3. ¹H NMR (400 MHz, METHANOL-d₄) δ ppm 8.32 (d, J=2.43 Hz, 1H), 8.29 (s, 1H), 7.87 (s, 1H), 7.75 (d, J=2.65 Hz, 1H), 5.09 (s, 2H), 4.61 (s, 2H), 4.06-4.13 (m, 2H), 3.60-4.02 (m, 15H), 3.52 (br d, J=16.98 Hz, 4H), 3.42 (s, 6H), 3.00 (s, 3H)

### Example 78

### 5-chloro-2-hydroxy-3-methyl-N-(4-((4-methylpiperazin-1-yl)methyl)-6-(trifluoromethyl)benzo[d]thiazol-2-yl)benzamide (78)

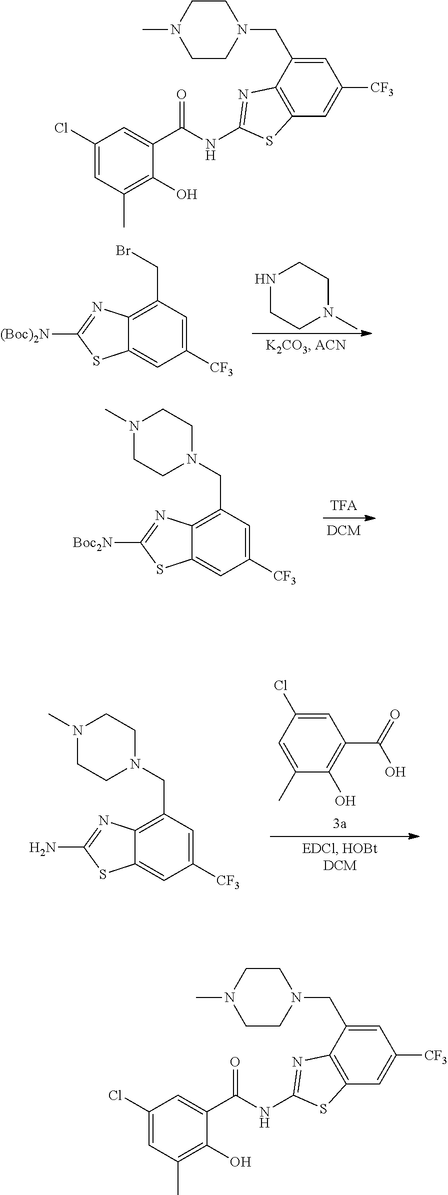

To a solution of 1-methylpiperazine (29 mg, 293.34 μmol, 32.54 μL) in MeCN (2.0 mL) was added K₂CO₃ (81 mg, 586.68 μmol) and the mixture was stirred at 0° C. for 10 minutes, then tert-butyl N-(4-(bromomethyl)-6-(trifluoromethyl)benzo[d]thiazol-2-yl)-N-tert-butoxycarbonylcarbamate (0.1 g, 195.56 μmol, Example 64) was added, and the mixture was stirred at 20° C. for 2 h. The reaction mixture was concentrated under reduced pressure to remove solvent. The residue was diluted with water (2 mL) and extracted with ethyl acetate (3 mL×2). The combined organic layer was washed with brine (5 mL), dried over Na₂SO₄, filtered and concentrated under reduced pressure to give a residue. The residue was purified by prep-TLC (SiO₂, DCM/MeOH=10/1) to afford tert-butyl N-tert-butoxycarbonyl-N-(4-((4-methylpiperazin-1-yl)methyl)-6-(trifluoromethyl)benzo[d]thiazol-2-yl)carbamate (100 mg, 144.93 μmol, 74.1% yield, 76.9% purity) as yellow oil.

To a solution of tert-butyl N-tert-butoxycarbonyl-N-(4-((4-methylpiperazin-1-yl)methyl)-6-(trifluoromethyl)benzo[d]thiazol-2-yl)carbamate (100 mg, 188.47 μmol) in DCM (2.0 mL) was added TFA (1 mL). The mixture was stirred at 20° C. for 1 h. The reaction mixture was pulled into sat. NaHCO₃ (10 mL), and then extracted with DCM (3 mL×4). The combined organic layer was washed with brine (10 mL×2), dried over Na₂SO₄, filtered and concentrated under reduced pressure to give 4-((4-methylpiperazin-1-yl)methyl)-6-(trifluoromethyl)benzo[d]thiazol-2-amine (50 mg, crude) as yellow oil.

A mixture of 4-((4-methylpiperazin-1-yl)methyl)-6-(trifluoromethyl)benzo[d]thiazol-2-amine (25 mg, 75.67 μmol), 5-chloro-2-hydroxy-3-methylbenzoic acid (16 mg, 90.81 μmol), HOBt (15 mg, 113.51 μmol), EDCI (17 mg, 90.81 μmol) in DCM (3 mL), was stirred at 45° C. for 12 h. The reaction mixture was concentrated under reduced pressure to remove solvent. The residue was purified by prep-HPLC (HCl condition) to afford 5-chloro-2-hydroxy-3-methyl-N-(4-((4-methylpiperazin-1-yl)methyl)-6-(trifluoromethyl)benzo[d]thiazol-2-yl)benzamide (15 mg, 100% purity, HCl) as a white solid. ¹H NMR (400 MHz, DMSO-d₆) δ ppm 8.61 (br s, 1H), 8.17 (br s, 1H), 8.08 (br s, 1H), 7.50 (br s, 1H), 4.68 (br s, 2H), 3.34-3.51 (m, 8H), 2.79 (br s, 3H), 2.24 (s, 3H); MS (ESI) [M+H]⁺ requires m/z 499.11, found m/z 499.1

### Example 79

### 5-chloro-3-((cis-2,6-dimethylmorpholino)methyl)-2-hydroxy-N-(4-((4-methylpiperazin-1-yl)methyl)-6-(trifluoromethyl)benzo[d]thiazol-2-yl)benzamide (79)

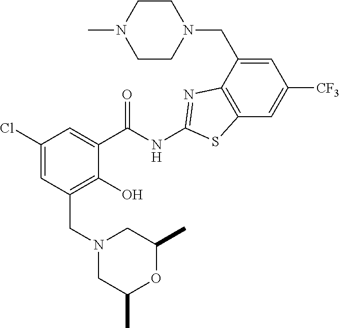 [SMILES: C[C@@H]1CN(Cc2cc(Cl)cc(C(=O)Nc3nc4c(CN5CCN(C)CC5)cc(C(F)(F)F)cc4s3)c2O)C[C@H](C)O1]

5-chloro-3-((cis-2,6-dimethylmorpholino)methyl)-2-hydroxy-N-(4-((4-methylpiperazin-1-yl)methyl)-6-(trifluoromethyl)benzo[d]thiazol-2-yl)benzamide was made in a manner similar to Example 78. LCMS: MS (ESI) [M+H]⁺ requires m/z 612.19, found m/z 612.2. ¹HNMR (400 MHz, METHANOL-d₄) δ ppm 8.47-8.56 (m, 2H), 8.02 (s, 1H), 7.86 (d, J=2.43 Hz, 1H), 4.89 (br s, 2H), 4.45 (s, 2H), 3.90 (br d, J=6.17 Hz, 2H), 3.74 (br s, 8H), 3.46 (br d, J=11.69 Hz, 2H), 3.01 (s, 3H), 2.80-2.92 (m, 2H), 1.24 (d, J=6.39 Hz, 6H)

### Example 80

### 3-((bis(2-methoxyethyl)amino)methyl)-5-chloro-2-hydroxy-N-(4-((4-methylpiperazin-1-yl)methyl)-6-(trifluoromethyl)benzo[d]thiazol-2-yl)benzamide (80)

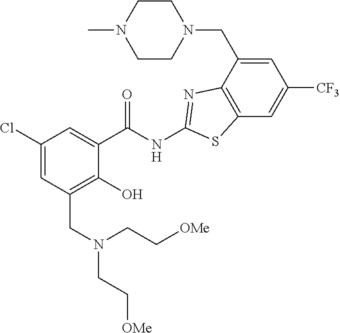 [SMILES: COCCN(CCOC)Cc1cc(Cl)cc(C(=O)Nc2nc3c(CN4CCN(C)CC4)cc(C(F)(F)F)cc3s2)c1O]

3-((bis(2-methoxyethyl)amino)methyl)-5-chloro-2-hydroxy-N-(4-((4-methylpiperazin-1-yl)methyl)-6-(trifluoromethyl)benzo[d]thiazol-2-yl)benzamide was made in a manner similar to Example 78. LCMS: MS (ESI) [M+H]⁺ requires m/z 630.21, found m/z 630.2. ¹H NMR (400 MHz, METHANOL-d₄) δ ppm 8.41-8.50 (m, 2H), 7.98 (s, 1H), 7.80 (d, J=2.43 Hz, 1H), 4.71 (br s, 2H), 4.61 (s, 2H), 3.79 (t, J=4.96 Hz, 4H), 3.47-3.73 (m, 12H), 3.42 (s, 6H), 2.98 (s, 3H)

### Example 81

### 5-chloro-2-hydroxy-3-((4-methylpiperazin-1-yl)methyl)-N-(4-((4-methylpiperazin-1-yl)methyl)-6-(trifluoromethyl)benzo[d]thiazol-2-yl)benzamide(81)

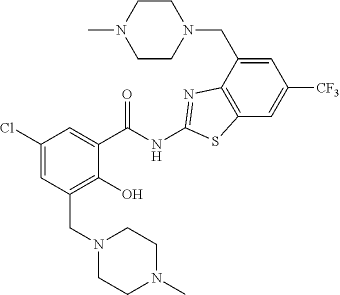 [SMILES: CN1CCN(Cc2cc(Cl)cc(C(=O)Nc3nc4c(CN5CCN(C)CC5)cc(C(F)(F)F)cc4s3)c2O)CC1]

5-chloro-2-hydroxy-3-((4-methylpiperazin-1-yl)methyl)-N-(4-((4-methylpiperazin-1-yl)methyl)-6-(trifluoromethyl)benzo[d]thiazol-2-yl)benzamide was made in a manner similar to Example 78. LCMS: MS (ESI) [M+H]⁺ requires m/z 597.19, found m/z 597.2. ¹HNMR (400 MHz, DMSO-d₆) δ ppm 11.87 (br s, 1H), 8.61 (s, 1H), 8.20 (br s, 1H), 8.13 (br s, 1H), 7.88 (br d, J=1.76 Hz, 1H), 4.74 (br s, 2H), 4.36 (br s, 2H), 3.61 (br d, J=7.28 Hz, 8H), 3.44 (br s, 8H), 2.82 (br d, J=12.13 Hz, 6H)

### Example 82

### 5-chloro-N-(4-(((4-chlorobenzyl)oxy)methyl)-6-(trifluoromethyl)benzo[d]thiazol-2-yl)-2-hydroxy-3-methylbenzamide (82)

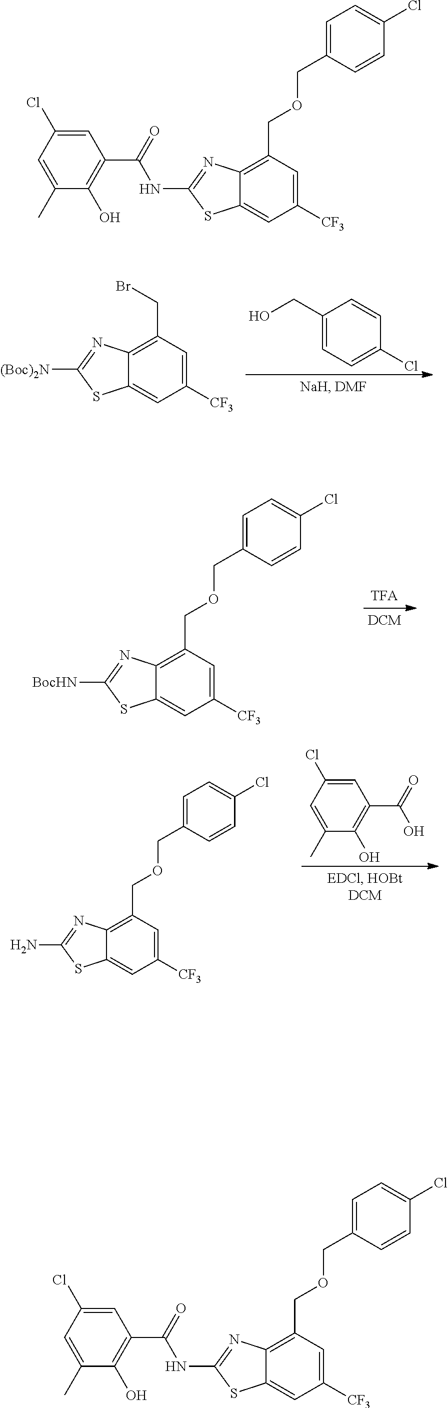

To a stirred solution of (4-chlorophenyl)methanol (83 mg, 586.68 μmol, 6.86 μL) in DMF (4.0 mL) at −10° C., was added NaH (117 mg, 2.93 mmol, 60% purity). After 1 h, tert-butyl N-(4-(bromomethyl)-6-(trifluoromethyl)benzo[d]thiazol-2-yl)-N-tert-butoxycarbonylcarbamate (200 mg, 293.34 μmol, Example 64) was added. Stirring was continued at −10° C. for 1 h. The reaction mixture was quenched by addition sat. NH₄Cl (5 mL), and then diluted with water (5 mL) and extracted with DCM (5 mL×3). The combined organic layer was washed with brine (10 mL), dried over Na₂SO₄, filtered and concentrated under reduced pressure to give a residue. The residue was purified by prep-TLC (SiO₂, petroleum ether/ethyl acetate=3/1) to afford tert-butyl (4-(((4-chlorobenzyl)oxy)methyl)-6-(trifluoromethyl)benzo[d]thiazol-2-yl)carbamate (72 mg, 86.70 μmol, 29.5% yield, 69% purity) as a yellow solid.

To a solution of tert-butyl N-tert-butoxycarbonyl-N-[4-[(4-chlorophenyl)methoxymethyl]-6-(trifluoromethyl)-1,3-benzothiazol-2-yl]carbamate (70 mg, 122.16 μmol) in DCM (2.0 mL) was added TFA (1 mL). The mixture was stirred at 20° C. for 5 h. The reaction mixture was concentrated under reduced pressure to remove solvent. The residue was diluted with H₂O (3 mL) and extracted with DCM (2 mL×2). The combined organic layer was washed with brine (5 mL), dried over Na₂SO₄, filtered and concentrated under reduced pressure to give a residue. The residue was purified by prep-TLC (SiO₂, petroleum ether/ethyl acetate=3/1) to afford 4-(((4-chlorobenzyl)oxy)methyl)-6-(trifluoromethyl)benzo[d]thiazol-2-amine (40 mg, 58.16 μmol, 47.6% yield, 54.2% purity) as a yellow solid.

To a solution of 4-(((4-chlorobenzyl)oxy)methyl)-6-(trifluoromethyl)benzo[d]thiazol-2-amine (40 mg, 107.30 μmol), 5-chloro-2-hydroxy-3-methylbenzoic acid (40 mg, 214.60 μmol) in DCM (4.0 mL) was added EDCI (24 mg, 128.76 μmol) and HOBt (21 mg, 160.95 μmol). The mixture was stirred at 50° C. for 12 h. The reaction mixture was concentrated under reduced pressure to remove solvent. The residue was purified by prep-HPLC (HCl condition) to afford 5-chloro-N-(4-(((4-chlorobenzyl)oxy)methyl)-6-(trifluoromethyl)benzo[d]thiazol-2-yl)-2-hydroxy-3-methylbenzamide (8.4 mg, 15.61 μmol, 14.5% yield, 100% purity) as a white solid. ¹H NMR (400 MHz, METHANOL-d₄) δ ppm 8.22 (s, 1H), 7.95 (d, J=2.45 Hz, 1H), 7.79 (s, 1H), 7.33-7.42 (m, 5H), 5.05 (s, 2H), 4.69 (s, 2H), 2.28 (s, 3H); MS (ESI) [M+H]⁺ requires m/z 541.03, found m/z 541.1.

### Example 83

### 5-chloro-N-(4-(((4-fluorobenzyl)oxy)methyl)-6-(trifluoromethyl)benzo[d]thiazol-2-yl)-2-hydroxy-3-methylbenzamide (83)

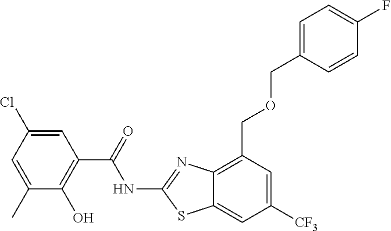 [SMILES: Cc1cc(Cl)cc(C(=O)Nc2nc3c(COCc4ccc(F)cc4)cc(C(F)(F)F)cc3s2)c1O]

5-chloro-N-(4-(((4-fluorobenzyl)oxy)methyl)-6-(trifluoromethyl)benzo[d]thiazol-2-yl)-2-hydroxy-3-methylbenzamide was made in a manner similar to Example 82. LCMS: MS (ESI) [M+H]⁺ requires m/z 525.06, found m/z 525.1. ¹H NMR (400 MHz, DMSO-d₆) δ ppm 8.47 (s, 1H), 8.03 (br s, 1H), 7.78 (s, 1H), 7.36-7.52 (m, 3H), 7.19 (t, J=8.80 Hz, 2H), 4.98 (s, 2H), 4.66 (s, 2H), 2.22 (s, 3H)

### Example 84

### N-(4-((benzyloxy)methyl)-6-(trifluoromethyl)benzo[d]thiazol-2-yl)-5-chloro-2-hydroxy-3-methylbenzamide (84)

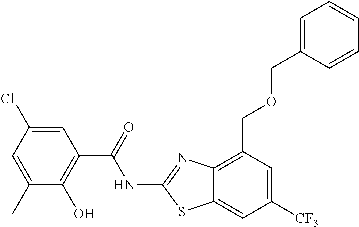 [SMILES: Cc1cc(Cl)cc(C(=O)Nc2nc3c(COCc4ccccc4)cc(C(F)(F)F)cc3s2)c1O]

N-(4-((benzyloxy)methyl)-6-(trifluoromethyl)benzo[d]thiazol-2-yl)-5-chloro-2-hydroxy-3-methylbenzamide was made in a manner similar to Example 82. LCMS: MS (ESI) [M+H]⁺ requires m/z 507.07, found m/z 507.2. ¹HNMR (400 MHz, DMSO-d₆) δ ppm 8.47 (s, 1H), 8.04 (br s, 1H), 7.80 (s, 1H), 7.48 (br s, 1H), 7.35-7.43 (m, 4H), 7.28-7.34 (m, 1H), 4.99 (s, 2H), 4.69 (s, 2H), 2.22 (s, 3H)

### Example 85

### 5-chloro-2-hydroxy-3-methyl-N-(4-(((4-methylbenzyl)oxy)methyl)-6-(trifluoromethyl)benzo[d]thiazol-2-yl)benzamide (85)

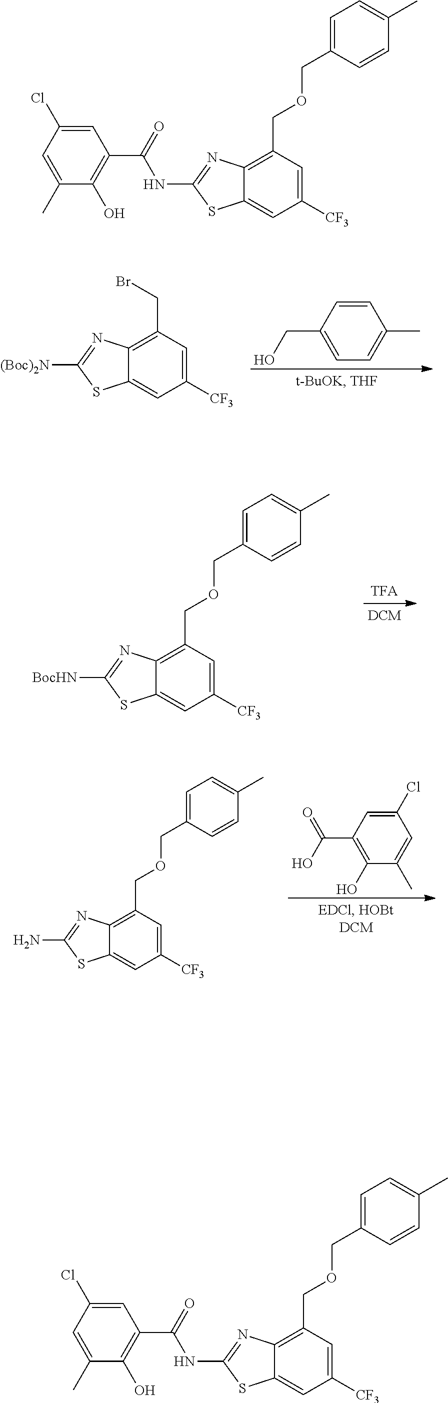

To a solution of tert-butyl N-(4-(bromomethyl)-6-(trifluoromethyl)benzo[d]thiazol-2-yl)-N-tert-butoxycarbonylcarbamate (0.2 g, 391.12 μmol, Example 64) and p-tolylmethanol (95 mg, 782.24 μmol, 63.25 μL) in THF (5 mL) was added t-BuOK, (1 M, 1.56 mL, THF), at 0° C., then the mixture was stirred at 20° C. for 1 h. The residue was diluted with water (5 mL) and extracted with ethyl acetate (3 mL×2). The combined organic layer was washed with brine (3 mL×2), dried over Na₂SO₄, filtered and concentrated under reduced pressure to give tert-butyl (4-(((4-methylbenzyl)oxy)methyl)-6-(trifluoromethyl)benzo[d]thiazol-2-yl)carbamate (150 mg, crude) as yellow oil.

To a solution of tert-butyl (4-(((4-methylbenzyl)oxy)methyl)-6-(trifluoromethyl)benzo[d]thiazol-2-yl)carbamate (150 mg, 331.50 μmol) in DCM (2.0 mL) was added TFA (1.0 mL). The mixture was stirred at 30° C. for 1 h. The reaction mixture was put into sat. NaHCO₃ (10 mL) and extracted with DCM (5 mL×2). The combined organic layer was washed with brine (20 mL×2), dried over Na₂SO₄, filtered and concentrated under reduced pressure to give a residue. The residue was purified by prep-TLC (SiO₂, petroleum ether/ethyl acetate=1/1) to afford 4-(((4-methylbenzyl)oxy)methyl)-6-(trifluoromethyl)benzo[d]thiazol-2-amine (50 mg, 76.62 μmol, 23.11% yield, 54% purity) as a yellow solid.

To a solution of 4-(((4-methylbenzyl)oxy)methyl)-6-(trifluoromethyl)benzo[d]thiazol-2-amine (30 mg, 85.14 μmol), 5-chloro-2-hydroxy-3-methylbenzoic acid (19 mg, 102.16 μmol) in DCM (2.0 mL) was added EDCI (19 mg, 102.16 μmol) and HOBt (17 mg, 127.71 μmol). The mixture was stirred at 45° C. for 12 h. The reaction mixture was concentrated under reduced pressure to remove solvent. The residue was purified by prep-HPLC (neutral condition) to afford 5-chloro-2-hydroxy-3-methyl-N-(4-(((4-methylbenzyl)oxy)methyl)-6-(trifluoromethyl)benzo[d]thiazol-2-yl)benzamide (10.9 mg, 97.23% purity) as a white solid. ¹H NMR (400 MHz, DMSO-d₆) δ ppm 8.46 (br s, 1H), 8.03 (br s, 1H), 7.77 (br s, 1H), 7.47 (br s, 1H), 7.28 (br d, J=7.50 Hz, 2H), 7.18 (br d, J=7.50 Hz, 2H), 4.96 (br s, 2H), 4.63 (s, 2H), 2.30 (s, 3H), 2.22 (s, 3H); MS (ESI) [M+H]⁺ requires m/z 521.08, found m/z 521.2.

### Example 86

### 5-chloro-N-(4-((2-(dimethylamino)ethoxy)methyl)-6-(trifluoromethyl)benzo[d]thiazol-2-yl)-2-hydroxy-3-methylbenzamide (86)

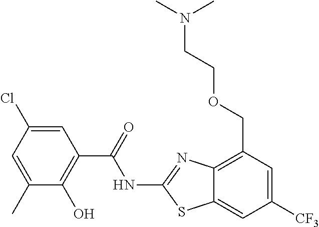 [SMILES: Cc1cc(Cl)cc(C(=O)Nc2nc3c(COCCN(C)C)cc(C(F)(F)F)cc3s2)c1O]

5-chloro-N-(4-((2-(dimethylamino)ethoxy)methyl)-6-(trifluoromethyl)benzo[d]thiazol-2-yl)-2-hydroxy-3-methylbenzamide was made in a manner similar to Example 85. LCMS: MS (ESI) [M+H]⁺ requires m/z 488.09, found m/z 488.0. ¹H NMR (400 MHz, DMSO-d₆) δ ppm 9.72 (br s, 1H), 8.19 (s, 1H), 7.69 (d, J=2.65 Hz, 1H), 7.62 (s, 1H), 7.21 (d, J=2.87 Hz, 1H), 4.97 (s, 2H), 3.90 (t, J=5.07 Hz, 2H), 3.42 (br t, J=4.96 Hz, 2H), 2.89 (s, 6H), 2.16 (s, 3H)

### Example 87

### 5-chloro-2-hydroxy-3-methyl-N-(4-((thiazol-2-ylmethoxy)methyl)-6-(trifluoromethyl)benzo[d]thiazol-2-yl)benzamide (87)

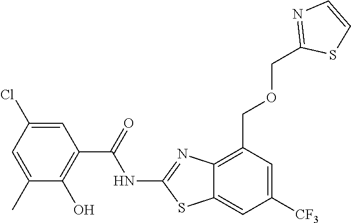 [SMILES: Cc1cc(Cl)cc(C(=O)Nc2nc3c(COCc4nccs4)cc(C(F)(F)F)cc3s2)c1O]

5-chloro-2-hydroxy-3-methyl-N-(4-((thiazol-2-ylmethoxy)methyl)-6-(trifluoromethyl)benzo[d]thiazol-2-yl)benzamide was made in a manner similar to Example 85. LCMS: MS (ESI) [M+H]⁺ requires m/z 514.02, found m/z 513.9. ¹HNMR (400 MHz, DMSO-d₆) δ ppm 8.49 (s, 1H), 8.04 (br s, 1H), 7.82 (d, J=3.09 Hz, 2H), 7.76 (d, J=3.31 Hz, 1H), 7.48 (br s, 1H), 5.11 (s, 2H), 5.00 (s, 2H), 2.22 (s, 3H)

### Example 88

### 5-chloro-2-hydroxy-N-(4-((2-hydroxyethoxy)methyl)-6-(trifluoromethyl)benzo[d]thiazol-2-yl)-3-methylbenzamide (88)

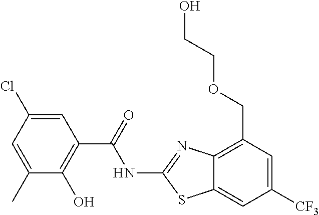 [SMILES: Cc1cc(Cl)cc(C(=O)Nc2nc3c(COCCO)cc(C(F)(F)F)cc3s2)c1O]

5-chloro-2-hydroxy-N-(4-((2-hydroxyethoxy)methyl)-6-(trifluoromethyl)benzo[d]thiazol-2-yl)-3-methylbenzamide was made in a manner similar to Example 85.LCMS: MS (ESI) [M+H]⁺ requires m/z 461.05, found m/z 460.9. ¹HNMR (400 MHz, DMSO-d₆) δ ppm 8.46 (s, 1H), 8.03 (br s, 1H), 7.83 (s, 1H), 7.48 (br s, 1H), 4.96 (br s, 2H), 3.61 (s, 4H), 2.23 (s, 3H)

### Example 89

### 5-chloro-2-hydroxy-N-(4-(((4-methoxybenzyl)oxy)methyl)-6-(trifluoromethyl)benzo[d]thiazol-2-yl)-3-methylbenzamide (89)

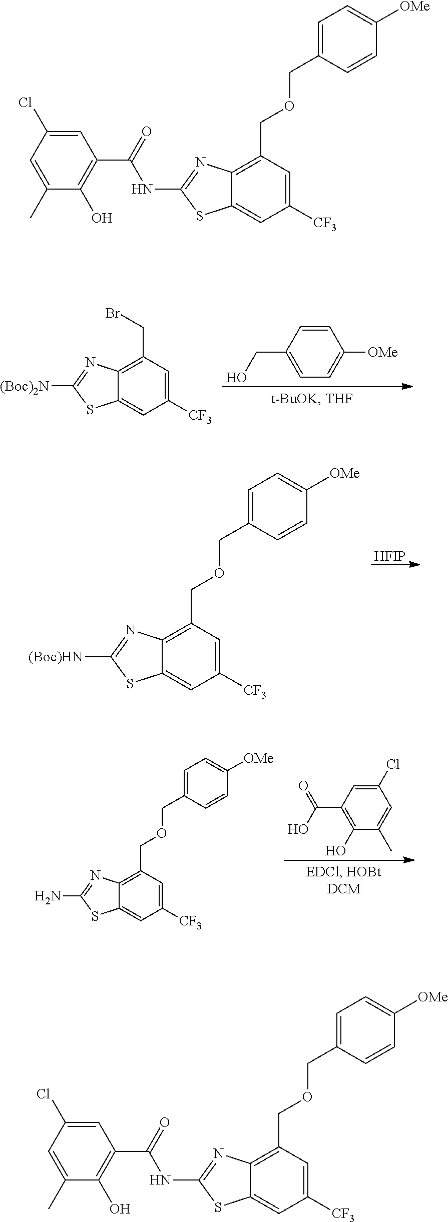

To a solution of tert-butyl N-(4-(bromomethyl)-6-(trifluoromethyl)benzo[d]thiazol-2-yl)-N-tert-butoxycarbonylcarbamate (0.4 g, 782.24 μmol, Example 64) and (4-methoxyphenyl)methanol (140 mg, 1.02 mmol, 126.58 μL) in THE (10.0 mL) was added tBuOK, (1 M, 3.13 mL, THE) at 0° C., then the mixture was stirred at 20° C. for 12 h. The residue was diluted with water (20 mL) and extracted with ethyl acetate (10 mL×2). The combined organic layer was washed with brine (20 mL×2), dried over Na₂SO₄, filtered and concentrated under reduced pressure to give a residue. The residue was purified by flash silica gel chromatography (ISCO®; 12 g SepaFlash® Silica Flash Column, Eluent of 0~10% ethyl acetate/petroleum ether gradient @70 mL/min) to afford tert-butyl (4-(((4-methoxybenzyl)oxy)methyl)-6-(trifluoromethyl)benzo[d]thiazol-2-yl)carbamate (170 mg, 362.87 μmol, 46.39% yield) as yellow oil.

A solution of tert-butyl (4-(((4-methoxybenzyl)oxy)methyl)-6-(trifluoromethyl)benzo[d]thiazol-2-yl)carbamate (100 mg, 213.45 μmol) in 1,1,1,3,3,3-hexafluoropropan-2-ol (3.0 mL) was stirred at 80° C. for 1.5 h. The reaction mixture was concentrated under reduced pressure to remove solvent. The residue was purified by prep-TLC (SiO₂, petroleum ether/ethyl acetate=3/1) to afford 4-(((4-methoxybenzyl)oxy)methyl)-6-(trifluoromethyl)benzo[d]thiazol-2-amine (70 mg, 190.02 μmol, 89.0% yield) as a yellow solid.

To a solution of 4-(((4-methoxybenzyl)oxy)methyl)-6-(trifluoromethyl)benzo[d]thiazol-2-amine (70 mg, 190.02 μmol), and 5-chloro-2-hydroxy-3-methylbenzoic acid (35 mg, 190.02 μmol) in DCM (3.0 mL) was added EDCI (43 mg, 228.03 μmol) and HOBt (38 mg, 285.04 μmol). The mixture was stirred at 45° C. for 3 h. The reaction mixture was concentrated under reduced pressure to remove solvent. The residue was purified by prep-HPLC (neutral condition) to afford 5-chloro-2-hydroxy-N-(4-(((4-methoxybenzyl)oxy)methyl)-6-(trifluoromethyl)benzo[d]thiazol-2-yl)-3-methylbenzamide (25 mg, 47.92 μmol, 25.2% yield, 100% purity) as a yellow solid. ¹H NMR (400 MHz, DMSO-d₆) δ ppm 8.45 (s, 1H), 8.03 (br s, 1H), 7.76 (s, 1H), 7.47 (br s, 1H), 7.31 (d, J=8.31 Hz, 2H), 6.92 (d, J=8.80 Hz, 2H), 4.94 (s, 2H), 4.60 (s, 2H), 3.74 (s, 3H), 2.22 (s, 3H); MS (ESI) [M+H]⁺ requires m/z 537.08, found m/z 537.0

### Example 90

### 5-chloro-2-hydroxy-3-methyl-N-(4-(propionamidomethyl)-6-(trifluoromethyl)benzo[d]thiazol-2-yl)benzamide (90)

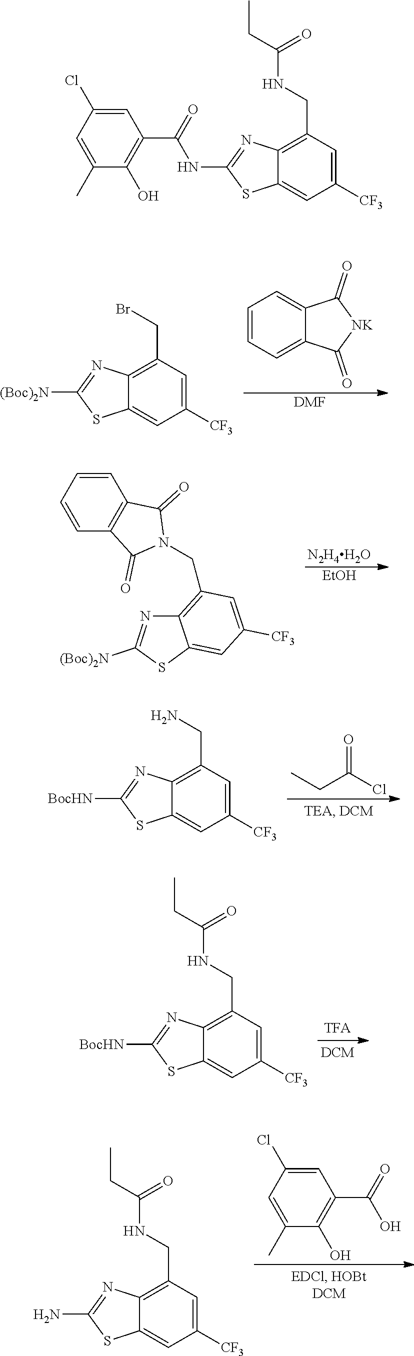

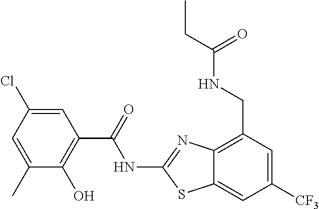 [SMILES: CCC(=O)NCc1cc(C(F)(F)F)cc2sc(NC(=O)c3cc(Cl)cc(C)c3O)nc12]

To a solution of tert-butyl N-(4-(bromomethyl)-6-(trifluoromethyl)benzo[d]thiazol-2-yl)-N-tert-butoxycarbonylcarbamate (300 mg, 586.68 μmol, Example 64) in DMF (5.0 mL) was added potassium 1,3-dioxoisoindolin-2-ide) (163 mg, 880.02 μmol). The mixture was stirred at 25° C. for 12 h. The reaction mixture was diluted with water (10 mL) and extracted with ethyl acetate (5 mL×3). The combined organic layer was washed with brine (15 mL×2), dried over Na₂SO₄, filtered and concentrated under reduced pressure to give a residue. The residue was purified by flash silica gel chromatography (ISCO®; 4 g SepaFlash® Silica Flash Column, eluent of 0~10% ethyl acetate/petroleum ether gradient @70 mL/min) to afford tert-butyl N-tert-butoxycarbonyl-N-(4-((1,3-dioxoisoindolin-2-yl)methyl)-6-(trifluoromethyl)benzo[d]thiazol-2-yl)carbamate (280 mg, 380.07 μmol, 64.78% yield, 78.4% purity) as a white solid.

To a suspension of tert-butyl N-tert-butoxycarbonyl-N-(4-((1,3-dioxoisoindolin-2-yl)methyl)-6-(trifluoromethyl)benzo[d]thiazol-2-yl)carbamate (280 mg, 484.79 μmol) in EtOH (5.0 mL) was added N₂H₄·H₂O (49 mg, 969.58 μmol, 48.09 μL, 98% purity). The mixture was stirred at 25° C. for 12 h. The reaction mixture was concentrated under reduced pressure to remove solvent. The residue was diluted with DCM (5 mL), then filtered and concentrated under reduced pressure to give tert-butyl (4-(aminomethyl)-6-(trifluoromethyl)benzo[d]thiazol-2-yl)carbamate (150 mg, crude) as a white solid.

To a solution of t tert-butyl (4-(aminomethyl)-6-(trifluoromethyl)benzo[d]thiazol-2-yl)carbamate (100 mg, 287.89 μmol) in DCM (4 mL) was added TEA (43 mg, 431.83 μmol, 60.11 uL), then propanoyl chloride (31 mg, 345.47 μmol, 31 ul) was added at 0° C. The resulting mixture was stirred at 20° C. for 1 h. The reaction mixture was concentrated under reduced pressure to remove solvent. The residue was purified by prep-TLC (SiO₂, petroleum ether/ethyl acetate 1/1) to afford tert-butyl (4-(propionamidomethyl)-6-(trifluoromethyl)benzo[d]thiazol-2-yl)carbamate (70 mg, 143.85 μmol, 49.97% yield, 82.9% purity) as a white solid.

To a solution of tert-butyl (4-(propionamidomethyl)-6-(trifluoromethyl)benzo[d]thiazol-2-yl)carbamate (70 mg, 173.52 μmol) in DCM (2.0 mL) was added TFA (0.5 mL). The mixture was stirred at 20° C. for 1 h. The reaction mixture was concentrated under reduced pressure to remove solvent. The residue was diluted with sat NaHCO₃ (5 mL) and extracted with DCM (3 mL×3). The combined organic layers were washed with brine (5 mL×3), dried over Na₂SO₄, filtered and concentrated under reduced pressure to give a residue. The residue was purified by prep-TLC (SiO₂, petroleum ether/ethyl acetate=0/1) to afford N-((2-amino-6-(trifluoromethyl)benzo[d]thiazol-4-yl)methyl)propionamide (40 mg, 105.51 μmol, 60.8% yield, 80% purity) as a white solid.

To a solution of N-((2-amino-6-(trifluoromethyl)benzo[d]thiazol-4-yl)methyl)propionam (25 mg, 82.43 μmol), and 5-chloro-2-hydroxy-3-methylbenzoic acid (21 mg, 115.40 μmol) in DCM (3.0 mL) was added EDCI (18 mg, 98.91 μmol) and HOBt (16 mg, 123.64 μmol). The mixture was stirred at 45° C. for 4 h. The reaction mixture was concentrated under reduced pressure to remove solvent. The residue was purified by prep-HPLC (neutral condition) to afford 5-chloro-2-hydroxy-3-methyl-N-(4-(propionamidomethyl)-6-(trifluoromethyl)benzo[d]thiazol-2-yl)benzamide (9.4 mg, 19.76 μmol, 23.9% yield, 99.18% purity) as a white solid. ¹H NMR (400 MHz, DMSO-d₆) δ ppm 8.48 (br t, J=5.62 Hz, 1H), 8.42 (s, 1H), 8.03 (br s, 1H), 7.58 (s, 1H), 7.48 (br s, 1H), 4.72 (br s, 2H), 2.19-2.26 (m, 5H), 1.06 (t, J=7.61 Hz, 3H); MS (ESI) [M+H]⁺ requires m/z 472.06, found m/z 471.9

### Example 91

### 5-chloro-2-hydroxy-3-methyl-N-(4-((2-(methylsulfonamido)ethoxy)methyl)-6-(trifluoromethyl)benzo[d]thiazol-2-yl)benzamide (91)

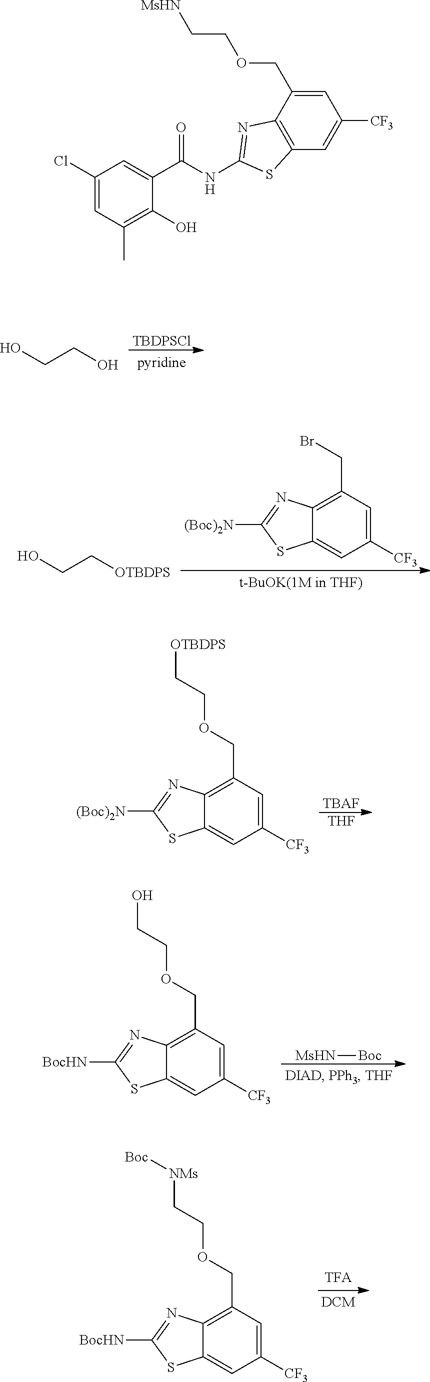

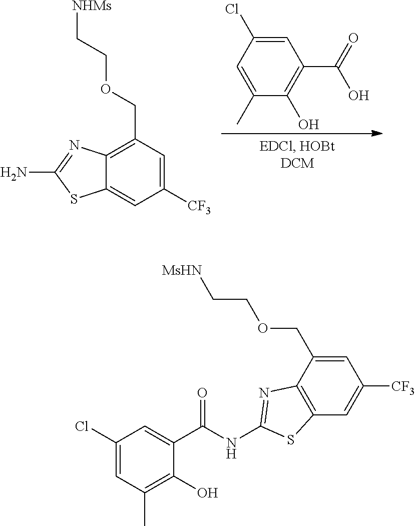

To a mixture of ethylene glycol (21.3 g, 343.92 mmol, 19.23 mL) and pyridine (20.0 mL) was added tert-butylchlorodiphenylsilane (10.7 g, 38.93 mmol, 10 mL), the mixture was stirred at 25° C. for 1 h. The reaction mixture was diluted with ethyl acetate (120 mL) and poured in water (60 mL), the layers were separated and the organic phase was washed with aq. NaHCO₃ (60 mL, Wt 10%) and water (60 mL), dried over Na₂SO₄, filtered and concentrated under reduced pressure to give a residue. The residue was purified by column chromatography (SiO₂, petroleum ether/ethyl acetate=1/0 to 10/1) to afford 2((tert-butyldiphenylsilyl)oxy)ethan-1-ol (9.8 g, 26.09 mmol, 67.03% yield, 80% purity) as yellow oil. ¹H NMR (400 MHz, CHLOROFORM-d) δ ppm 7.62-7.75 (m, 4H), 7.29-7.52 (m, 6H), 3.75-3.81 (m, 2H), 3.66-3.72 (m, 2H), 1.06-1.09 (m, 9H)

To a solution of 2((tert-butyldiphenylsilyl)oxy)ethan-1-ol (1.7 g, 5.87 mmol), tert-butyl N-(4-(bromomethyl)-6-(trifluoromethyl)benzo[d]thiazol-2-yl)-N-tert-butoxycarbonylcarbamate (1.5 g, 2.93 mmol, Example 64) in THE (60 mL) was added t-BuOK (1 M, 11.73 mL, in THF), at 0° C., then the mixture was stirred at 20° C. for 1 h. The residue was diluted with water (20 mL) and extracted with ethyl acetate (20 mL×2). The combined organic layer was washed with brine (20 mL), dried over Na₂SO₄, filtered and concentrated under reduced pressure to give tert-butyl N-tert-butoxycarbonyl-(4-((2-(tert-butyldiphenylsilyl)oxy)ethoxymethyl)-6-(trifluoromethyl)benzo[d]thiazol-2-yl)carbamate (2 g, crude) as yellow oil.

tert-butyl N-tert-butoxycarbonyl-(4-((2-(tert-butyldiphenylsilyl)oxy)ethoxymethyl)-6-(trifluoromethyl)benzo[d]thiazol-2-yl)carbamate (1 g, 1.37 mmol) was dissolved in THE (10 mL) and cooled to 0° C. then treated with TBAF (1 M, 2.74 mL). The resulting mixture was stirred at 0° C. for 5 minutes, then warmed to 20° C. and stirred for 12 h. The reaction mixture was diluted with water (20 mL) and extracted with ethyl acetate (10 mL×3). The combined organic layer was washed with brine (10 mL×3), dried over Na₂SO₄, filtered and concentrated under reduced pressure to give a residue. The residue was purified by flash silica gel chromatography (ISCO®; 12 g SepaFlash® Silica Flash Column, Eluent of 0~50% ethyl acetate/petroleum ether gradient @70 mL/min) to afford tert-butyl (4-((2-hydroxyethoxy)methyl)-6-(trifluoromethyl)benzo[d]thiazol-2-yl)carbamate (500 mg, 90.7% purity) as a white solid.

A solution of tert-butyl(methylsulfonyl)carbamate (29 mg, 152.28 μmol) in THF (3 mL) was added PPh₃ (79 mg, 304.56 μmol). The solution was stirred under N₂ and the tert-butyl tert-butyl N-tert-butoxycarbonyl-N-[4-(2-hydroxyethoxymethyl)-6-(trifluoromethyl)-1,3-benzothiazol-2-yl]carbamate (50 mg, 101.52 μmol) was added followed by DIAD (51 mg, 253.80 μmol, 49.35 μL). The mixture was stirred at 20° C. for 12 h. The reaction mixture was concentrated under reduced pressure to remove solvent. The residue was purified by prep-TLC (SiO₂, petroleum ether/ethyl acetate=3/1) to afford tert-butyl (2-((2-((tert-butoxycarbonyl)amino)-6-(trifluoromethyl)benzo[d]thiazol-4-yl)methoxy)ethyl)(methylsulfonyl)carbamate (60 mg, 75.88 μmol, 74.74% yield, 84.7% purity) as a white solid.

To a solution of tert-butyl (2-((2-((tert-butoxycarbonyl)amino)-6-(trifluoromethyl)benzo[d]thiazol-4-yl)methoxy)ethyl)(methylsulfonyl)carbamate (60 mg, 89.59 μmol) in DCM (2.0 mL) was added TFA (1 mL). The mixture was stirred at 25° C. for 3 h. The reaction mixture was poured into sat NaHCO₃ (5 mL), the pH was adjusted with sat. NaHCO₃ to around 9 and extracted with DCM (3 mL×3). The combined organic layer was washed with brine (5 mL×3), dried over Na₂SO₄, filtered and concentrated under reduced pressure to give N-(2-((2-amino-6-(trifluoromethyl)benzo[d]thiazol-4-yl)methoxy)ethyl)methanesulfonamide (30 mg, crude) as a yellow solid.

To a solution of N-(2-((2-amino-6-(trifluoromethyl)benzo[d]thiazol-4-yl)methoxy)ethyl)methanesulfonamide (30 mg, 81.22 μmol), 5-chloro-2-hydroxy-3-methylbenzoic acid (22 mg, 121.82 μmol) in DCM (3.0 mL) was added EDCI (18 mg, 97.46 μmol) and HOBt (16 mg, 121.82 μmol). The mixture was stirred at 45° C. for 5 h. The reaction mixture was concentrated under reduced pressure to remove solvent. The residue was purified by prep-HPLC (HCl condition) to afford 5-chloro-2-hydroxy-3-methyl-N-(4-((2-(methylsulfonamido)ethoxy)methyl)-6-(trifluoromethyl)benzo[d]thiazol-2-yl)benzamide (4.1 mg, 7.70 μmol, 9.4% yield, 100% purity) as a white solid. ¹H NMR (400 MHz, DMSO-d₆) δ ppm 8.47 (s, 1H), 8.02 (br s, 1H), 7.83 (s, 1H), 7.48 (br s, 1H), 7.12-7.23 (m, 1H), 4.96 (br s, 2H), 3.64 (br t, J=5.73 Hz, 2H), 3.13-3.27 (m, 2H), 2.91 (s, 3H), 2.23 (s, 3H); MS (ESI) [M+H]⁺ requires m/z 538.04, found m/z 538.0.

### Example 92

### 5-chloro-3-((cis-2,6-dimethylmorpholino)methyl)-2-hydroxy-N-(4-((2-(methylsulfonamido)ethoxy)methyl)-6-(trifluoromethyl)benzo[d]thiazol-2-yl)benzamide (92)

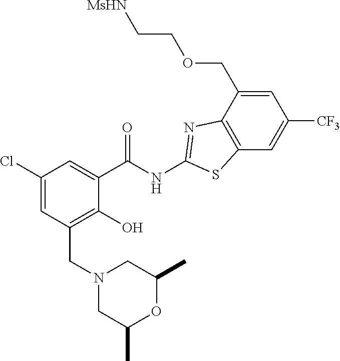

5-chloro-3-((cis-2,6-dimethylmorpholino)methyl)-2-hydroxy-N-(4-((2-(methylsulfonamido)ethoxy)methyl)-6-(trifluoromethyl)benzo[d]thiazol-2-yl)benzamide was made in a manner similar to Example 91. LCMS: MS (ESI) [M+H]⁺ requires m/z 651.12, found m/z 651.2. ¹H NMR (400 MHz, DMSO-d₆) δ ppm 8.36 (s, 1H), 7.87 (br s, 1H), 7.74 (s, 1H), 7.46 (br s, 1H), 7.17 (t, J=5.84 Hz, 1H), 4.93 (s, 2H), 4.25 (s, 2H), 3.90 (br dd, J=9.81, 6.73 Hz, 2H), 3.64 (t, J=5.73 Hz, 2H), 3.32 (br d, J=12.13 Hz, 2H), 3.20 (q, J=5.59 Hz, 2H), 2.91 (s, 3H), 2.63-2.74 (m, 2H), 1.12 (d, J=6.17 Hz, 6H)

### Example 93

### 3-((bis(2-methoxyethyl)amino)methyl)-5-chloro-2-hydroxy-N-(4-((2-(methylsulfonamido)ethoxy)methyl)-6-(trifluoromethyl)benzo[d]thiazol-2-yl)benzamide (93)

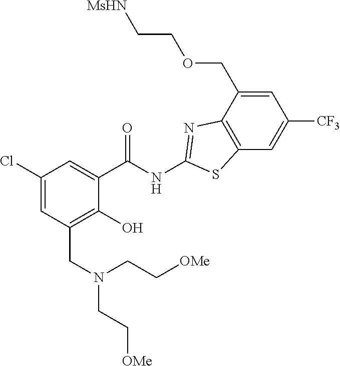

3-((bis(2-methoxyethyl)amino)methyl)-5-chloro-2-hydroxy-N-(4-((2-(methylsulfonamido)ethoxy)methyl)-6-(trifluoromethyl)benzo[d]thiazol-2-yl)benzamide was made in a manner similar to Example 91. LCMS: MS (ESI) [M+H]⁺ requires m/z 669.14, found m/z 669.2. ¹H NMR (400 MHz, METHANOL-d₄) δ ppm 8.21 (s, 1H), 8.08 (br s, 1H), 7.79 (s, 1H), 7.49 (br s, 1H), 5.00 (s, 2H), 4.47 (s, 2H), 3.75 (dt, J=8.71, 5.13 Hz, 6H), 3.42-3.45 (m, 4H), 3.41 (s, 6H), 3.35 (t, J=5.29 Hz, 2H), 2.97 (s, 3H).

### Example 94

### 5-chloro-2-hydroxy-3-methyl-N-(4-(1-methylpiperidin-4-yl)-6-(trifluoromethyl)benzo[d]thiazol-2-yl)benzamide (94)

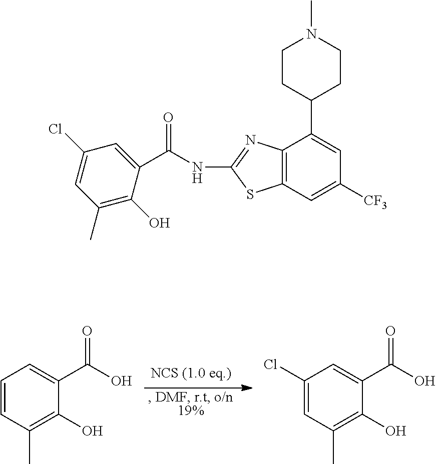 [SMILES: Cc1cc(Cl)cc(C(=O)Nc2nc3c(C4CCN(C)CC4)cc(C(F)(F)F)cc3s2)c1O ; Cc1cccc(C(=O)O)c1O ; ", DMF, r.t, o/n19%" ; "NCS (1.0 eq.)" ; Cc1cc(Cl)cc(C(=O)O)c1O]

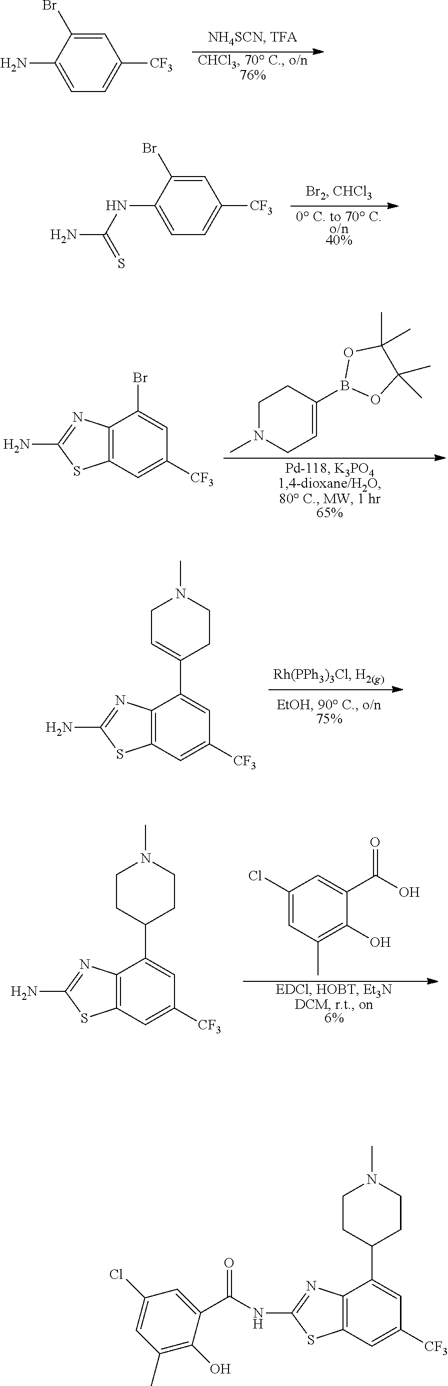

To a solution of 2-hydroxy-3-methylbenzoic acid (3.04 g, 20.0 mmol) in DMF (50 mL) was added NCS (2.67 g, 20.0 mmol), and the mixture was stirred at room temperature overnight. After completion of the reaction as indicated by LCMS, the reaction mixture was poured into H₂O (150 mL), extracted with ethyl acetate (200 mL×4) and concentrated under reduced pressure to give a residue, which was purified by prep-HPLC to give 5-chloro-2-hydroxy-3-methylbenzoic acid (704.2 mg, 19% yield) as an off-white solid. MS Calcd.: 186.0; MS Found: 187.1 [M+H]⁺.

To a solution of 2-bromo-4-(trifluoromethyl) aniline (23.9 g, 100.0 mmol) and NH₄SCN (15.2 g, 200.0 mmol) in CHCl₃ (500 mL) was added TFA (17.1 g, 150.0 mmol), and the mixture was stirred at 70° C. overnight. After completion of the reaction as indicated by LCMS, the reaction mixture was filtered and the filtrate was concentrated under reduced pressure to give a residue, which was purified by silica gel column chromatography (eluted with 5% MeOH/DCM) to afford 1-(2-bromo-4-(trifluoromethyl)phenyl)thiourea (22.5 g, 76% yield) as a yellow solid. MS Calcd.: 297.9; MS Found: 299.0 [M+H]⁺.

To a solution of 1-(2-bromo-4-(trifluoromethyl)phenyl)thiourea (14.9 g, 50.0 mmol) in CHCl₃ (300 mL) was added dropwise Br₂ (15.8 g, 100.0 mmol) at 0° C., and then the mixture was stirred at 70° C. overnight. After completion of the reaction as indicated by LCMS, the reaction mixture was filtered. The solid was dissolved in water, adjusted to pH 10 with 2N NaOH (aq), and extracted with ethyl acetate. The combined organic extract was washed with brine, dried over Na₂SO₄ and concentrated under reduced pressure to give a residue, which was purified by silica gel column chromatography (eluted with 5% MeOH/DCM) to give 4-bromo-6-(trifluoromethyl)benzo[d]thiazol-2-amine (6.0 g, 40% yield) as a yellow solid. MS Calcd.: 295.9; MS Found: 297.0 [M+H]⁺.

To a solution of 4-bromo-6-(trifluoromethyl)benzo[d]thiazol-2-amine (592.0 mg, 2.0 mmol), 1-methyl-4-(4,4,5,5-tetramethyl-1,3,2-dioxaborolan-2-yl)-1,2,3,6-tetrahydropyridine (669.0 mg, 3.0 mmol) and K₃PO₄ (1.27 g, 6.0 mmol) in 1,4-dioxane (10 mL) and H₂O (1 mL) was added 1,1′-Bis(di-t-butylphosphino) ferrocene palladium dichloride (118.4 mg, 20% w/w), and then the mixture was stirred at 80° C. in a microwave reactor for 1 hour. After completion of the reaction as indicated by LCMS, the reaction mixture concentrated under reduced pressure to give a residue, which was purified by silica gel column chromatography (eluted with 5% MeOH/DCM) to give 4-(1-methyl-1,2,3,6-tetrahydropyridin-4-yl)-6-(trifluoromethyl)benzo[d]thiazol-2-amine (412.5 mg, 65% yield) as yellow solid. MS Calcd.: 313.1; MS Found: 314.2 [M+H]⁺.

To a solution of 4-(1-methyl-1,2,3,6-tetrahydropyridin-4-yl)-6-(trifluoromethyl)benzo[d]thiazol-2-amine (412.5 mg, 1.3 mmol) in EtOH (40 mL) was added Rh(PPh₃)₃Cl (412.5 mg), and the mixture was stirred at 90° C. under H₂ (g) at atmospheric pressure overnight. After completion of the reaction as indicated by LCMS, the reaction mixture was filtered through Celite and the filtrate was concentrated under reduced pressure to give a residue, which was purified by silica gel column chromatography (eluted with 10% MeOH/DCM) to give 4-(1-methylpiperidin-4-yl)-6-(trifluoromethyl)benzo[d]thiazol-2-amine (312.5 mg, 75% yield) as a light yellow solid. MS Calcd.: 315.1; MS Found: 316.0 [M+H]⁺.

To a solution of 4-(1-methylpiperidin-4-yl)-6-(trifluoromethyl)benzo[d]thiazol-2-amine (312.5 mg, 0.99 mmol), 5-chloro-2-hydroxy-3-methylbenzoic acid (202.5 mg, 1.09 mmol) and Et₃N (303.0 mg, 33.0 mmol) in DCM (5 mL) was added EDCI (382.2 mg, 2.0 mmol) and HOBT (270.4 mg, 2.0 mmol), and the mixture was stirred at room temperature overnight. After completion of the reaction as indicated by LCMS, the reaction mixture was concentrated under reduced pressure to give a residue, which was purified by silica gel column chromatography (eluted with 10% MeOH/DCM), followed by prep-HPLC to give 5-chloro-2-hydroxy-3-methyl-N-(4-(1-methylpiperidin-4-yl)-6-(trifluoromethyl)benzo[d]thiazol-2-yl)benzamide (28.86 mg, 6% yield) as a white solid. MS Calcd.: 483.1; MS Found: 484.0 [M+H]⁺. ¹H NMR (400 MHz, DMSO-d₆) δ 17.05 (d, J=2.8 Hz, 1H), 9.27 (dd, J=8.8, 12.0 Hz, 1H), 8.12 (s, 1H), 7.62 (d, J=2.8 Hz, 1H), 7.32 (d, J=12.0 Hz, 1H), 7.18 (d, J=2.8 Hz, 1H), 3.69-3.67 (m, 1H), 3.65-3.61 (m, 2H), 3.21 (s, 2H), 2.33 (s, 3H), 2.15-2.07 (m, 4H).

### Example 95

### 5-chloro-2-hydroxy-3-methyl-N-(4-(morpholinomethyl)-6-(trifluoro-methyl)benzo[d]thiazol-2-yl)benzamide (95)

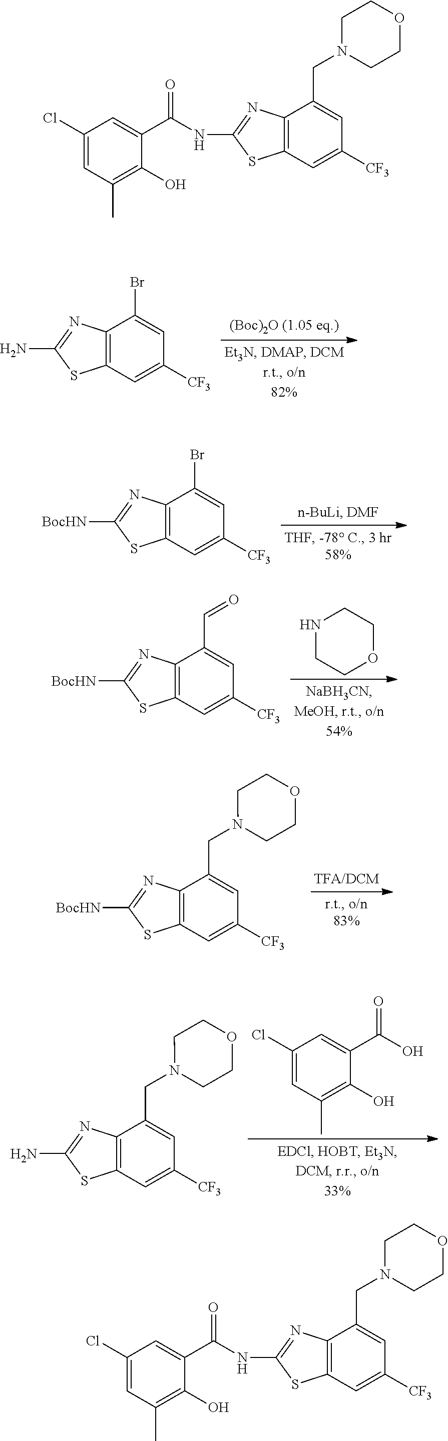

To a solution of 4-bromo-6-(trifluoromethyl)benzo[d]thiazol-2-amine (5.92 g, 20.0 mmol, Example 94), triethylamine (2.1 g, 21.0 mmol), DMAP (424.2 mg, 2.0 mmol) in DCM (200 mL) was added (Boc)₂O (4.6 g, 21.0 mmol), and the mixture was stirred at room temperature overnight. After completion of the reaction as indicated by LCMS, the reaction mixture was concentrated under reduced pressure to give the residue, which was purified by silica gel column chromatography (eluted with 10% ethy; acetate/petroleum ether) to give tert-butyl (4-bromo-6-(trifluoromethyl)benzo[d]thiazol-2-yl)carbamate (6.5 g, 82% yield) as a light-yellow solid. MS Calcd.: 396.0; MS Found: 341 [M−56+H]⁺.

To a solution of tert-butyl (4-bromo-6-(trifluoromethyl)benzo[d]thiazol-2-yl)carbamate (3.96 g, 10.0 mmol) in THF (50 mL) at −78° C. was added n-BuLi (2.5 N in hexane, 8.0 mL, 20.0 mmol), and the mixture was stirred at −78° C. for 1 hour. To this mixture was added DMF (1.48 g, 20.0 mmol), and the resulting mixture was stirred at −78° C. for another 1 hour. After completion of the reaction as indicated by LCMS, the reaction mixture was poured into sat. aq. NH₄Cl (100 mL), extracted with ethyl acetate (100 mL×4) and concentrated under reduced pressure to give a residue, which was purified by silica gel column chromatography (eluted with 20% ethyl acetate/petroleum ether) to give tert-butyl (4-formyl-6-(trifluoromethyl)benzo[d]thiazol-2-yl)carbamate (2.03 g, 58% yield) as a light-yellow solid. MS Calcd.: 346.1; MS Found: 291.0 [M−56+H]⁺.

To a solution of tert-butyl (4-formyl-6-(trifluoromethyl)benzo[d]thiazol-2-yl)carbamate (346.0 mg, 1.0 mmol) and morpholine (435.0 mg, 5.0 mmol) in CH₃OH (10 mL) was added NaBH₃CN (126 mg, 2.0 mmol). The mixture was stirred at room temperature overnight. After completion of the reaction as indicated by LCMS, the reaction mixture was poured into water, extracted with ethyl acetate (30 mL×4) and concentrated under reduced pressure to give a residue, which was purified by silica gel column chromatography (eluted with 33% ethyl acetate/petroleum ether) to give tert-butyl (4-(morpholinomethyl)-6-(trifluoromethyl)benzo[d]thiazol-2-yl)carbamate (226.3 mg, 54% yield) as a light-yellow solid. MS Calcd.: 417.1; MS Found: 418.0 [M+H]⁺.

To a solution of tert-butyl (4-(morpholinomethyl)-6-(trifluoromethyl)benzo[d]thiazol-2-yl)carbamate (226.3 mg, 0.54 mmol) in DCM (8 mL) was added TFA (2 mL) and the resulting solution was stirred at room temperature overnight. After completion of the reaction as indicated by LCMS, the reaction mixture was concentrated, poured into 0.05 N NaOH aq. (20 mL), extracted with DCM (30 mL×4) and concentrated under reduced pressure to give a residue, which was purified by silica gel column chromatography (eluted with 5% MeOH/DCM) to give 4-(morpholinomethyl)-6-(trifluoromethyl)benzo[d]thiazol-2-amine (143.6 mg, 83% yield) as a light-yellow solid. MS Calcd.: 317.1; MS Found: 318.0 [M+H]⁺.

To a solution of 4-(morpholinomethyl)-6-(trifluoromethyl)benzo[d]thiazol-2-amine (143.6 mg, 0.45 mmol), 5-chloro-2-hydroxy-3-methylbenzoic acid (92.7 mg, 0.50 mmol) and Et₃N (136.4 mg, 1.35 mmol) in DCM (5 mL) was added EDCI (171.9 mg, 0.90 mmol) and HOBT (121.5 mg, 0.90 mmol), and the mixture was stirred at room temperature overnight. After completion of the reaction as indicated by LCMS, the reaction mixture was concentrated under reduced pressure to give a residue, which was purified by silica gel column chromatography (eluted with 10% MeOH/DCM), followed by prep-HPLC to give 5-chloro-2-hydroxy-3-methyl-N-(4-(morpholinomethyl)-6-(trifluoromethyl)benzo[d]thiazol-2-yl)benzmide (70.8 mg, 33% yield) as a light-yellow solid. MS Calcd.: 485.1; MS Found: 486.1 [M+H]⁺. ¹H NMR (400 MHz, DMSO-d₆) δ 8.36 (s, 1H), 7.80 (s, 1H), 7.74 (s, 1H), 7.26 (d, J=2.0 Hz, 1H), 4.63 (s, 2H), 3.78 (s, 4H), 3.18 (d, J=7.2 Hz, 4H), 2.18 (s, 3H).

### Example 96

### 5-chloro-N-(4-((dimethylamino)methyl)-6-(trifluoromethyl)benzo[d]thiazol-2-yl)-2-hydroxy-3-methylbenzamide (96)

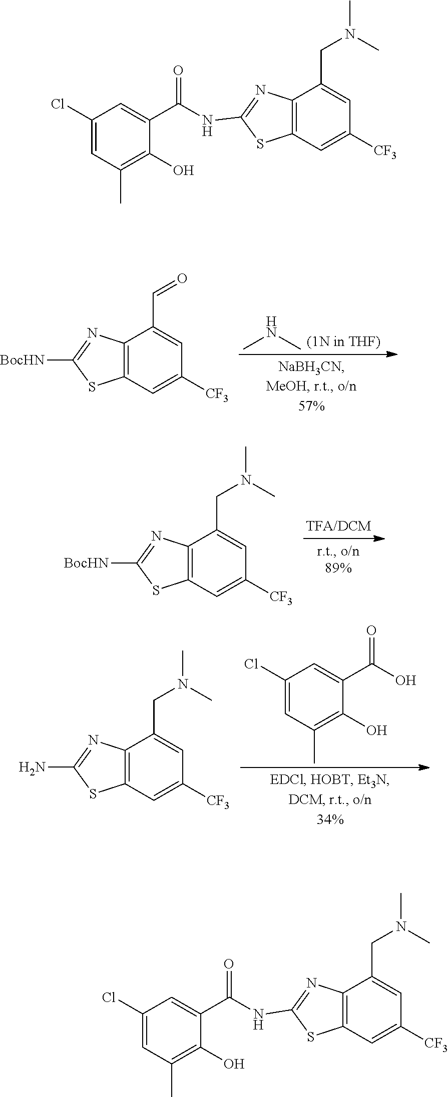

To a solution of tert-butyl (4-formyl-6-(trifluoromethyl)benzo[d]thiazol-2-yl)carbamate (346.0 mg, 1.0 mmol, Example 95) and dimethylamine (2.5 mL, 2 N in THF) in CH₃OH (10 mL) was added NaBH₃CN (126 mg, 2.0 mmol), and the mixture was stirred at room temperature overnight. After completion of the reaction indicated by LCMS, the reaction mixture was poured on water, extracted with ethyl acetate (30 mL×4) and concentrated under reduced pressure to give a residue, which was purified by silica gel column chromatography (eluted with 33% ethyl acetate/petroleum ether) to give tert-butyl (4-((dimethylamino)methyl)-6-(trifluoromethyl)benzo[d]thiazol-2-yl)carbamate (213.5 mg, 57% yield) as a light-yellow solid. MS Calcd.: 375.1; MS Found: 376.0 [M+H]⁺.

To a solution of tert-butyl (4-((dimethylamino)methyl)-6-(trifluoromethyl)benzo[d]thiazol-2-yl)carbamate (213.5 mg, 0.57 mmol) in DCM (8 mL) was added TFA (2 mL). The resulting solution was stirred at room temperature overnight. After completion of the reaction as indicated by LCMS, the reaction mixture was concentrated, poured into 0.05 N NaOH aq. (20 mL), extracted with DCM (30 mL×4) and concentrated under reduced pressure to give a residue, which was purified by silica gel column chromatography (eluted with 5% MeOH/DCM) to give 4-((dimethylamino)methyl)-6-(trifluoromethyl)benzo[d]thiazol-2-amine (140.2 mg, 89% yield) as a light-yellow solid. MS Calcd.: 275.1; MS Found: 276.1 [M+H]⁺.

To a solution of 4-((dimethylamino)methyl)-6-(trifluoromethyl)benzo[d]thiazol-2-amine (123.8 mg, 0.45 mmol), 5-chloro-2-hydroxy-3-methylbenzoic acid (92.7 mg, 0.50 mmol) and Et₃N (136.4 mg, 1.35 mmol) in DCM (5 mL) was added EDCI (171.9 mg, 0.90 mmol) and HOBT (121.5 mg, 0.90 mmol). The resulting mixture was stirred at room temperature overnight. After completion of the reaction as indicated by LCMS, the reaction mixture was concentrated under reduced pressure to give a residue, which was purified by silica gel column chromatography (eluted with 10% MeOH/DCM), followed by prep-HPLC to give 5-chloro-N-(4-((dimethylamino)methyl)-6-(trifluoromethyl)benzo[d]thiazol-2-yl)-2-hydroxy-3-methylbenzamide (68.4 mg, 34% yield) as an off-white solid. MS Calcd.: 443.1; MS Found: 444.1 [M+H]⁺. ¹H NMR (400 MHz, DMSO-d₆) δ 16.44 (s, 1H), 9.53 (br, 1H), 8.33 (s, 1H), 7.78 (s, 1H), 7.70 (d, J=2.4 Hz, 1H), 7.22 (d, J=2.4 Hz, 1H), 4.69 (s, 2H), 2.03 (s, 6H), 2.17 (s, 3H).

### Example 97

### 5-chloro-2-hydroxy-3-methoxy-N-(6-(trifluoromethyl)benzo[d]thiazol-2-yl)benzamide (97)

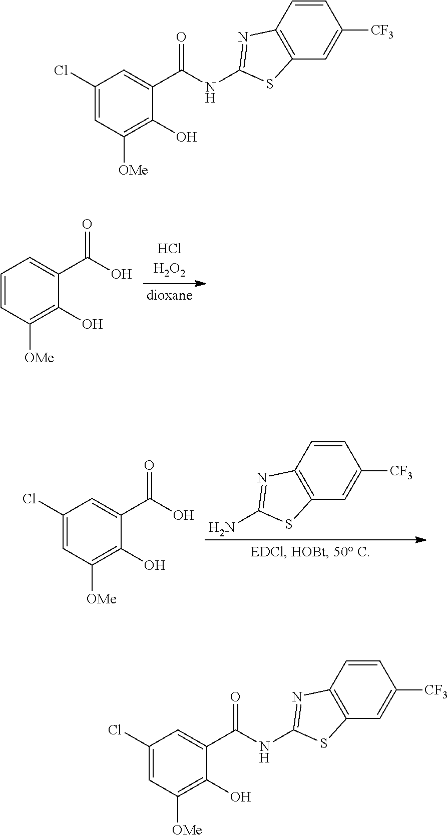 [SMILES: COc1cc(Cl)cc(C(=O)Nc2nc3ccc(C(F)(F)F)cc3s2)c1O ; COc1cccc(C(=O)O)c1O ; COc1cc(Cl)cc(C(=O)O)c1O ; Nc1nc2ccc(C(F)(F)F)cc2s1 ; COc1cc(Cl)cc(C(=O)Nc2nc3ccc(C(F)(F)F)cc3s2)c1O]

To a solution of 2-hydroxy-3-methoxybenzoic acid (200 mg, 1.19 mmol) in dioxane (2 mL) was added HCl (6 M, 208.15 μL). Then H₂O₂ (148 mg, 1.31 mmol, 125.72 μL, 30% purity) was added with rapid stirring of 1 minute, and then the mixture was stirred at 20° C. for 12 h. The reaction mixture was quenched by addition 10% Na₂SO₃ (5 mL), concentrated under reduced pressure to remove solvent, diluted with water (5 mL) and adjusted to pH 9 with 2 N NaOH. then extracted with ethyl acetate (5 mL×3). The clear solution was acidified with 1N HCl to pH 5 and then extracted with ethyl acetate (10 mL×3). The combined organic layer was washed with brine (50 mL), dried over Na₂SO₄, filtered and concentrated under reduced pressure to give 5-chloro-2-hydroxy-3-methoxybenzoic acid (140 mg, crude) as a yellow solid. MS (ESI) [M−H]⁻ requires m/z 201.00, found m/z 200.9

A mixture of 5-chloro-2-hydroxy-3-methoxybenzoic acid (100 mg, 493.60 μmol), 6-(trifluoromethyl)benzo[d]thiazol-2-amine (107 mg, 493.60 μmol), EDCI (94 mg, 493.60 μmol), and HOBt (66 mg, 493.60 μmol) in DCM (3 mL), was stirred at 50° C. for 12 h. The reaction mixture was concentrated under reduced pressure to remove solvent. The residue was purified by prep-HPLC (HCl condition) to afford 5-chloro-2-hydroxy-3-methoxy-N-(6-(trifluoromethyl)benzo[d]thiazol-2-yl)benzamide (59.49 mg, 100% purity) as a white solid. ¹H NMR (400 MHz, DMSO-d₆) δ ppm 8.54 (s, 1H), 7.90 (br d, J=7.94 Hz, 1H), 7.79 (br d, J=8.60 Hz, 1H), 7.52 (d, J=2.43 Hz, 1H), 7.28 (s, 1H), 3.90 (s, 3H). MS (ESI) [M−H]⁻ requires m/z 403.01, found m/z 402.9

### Example 98

### 5-chloro-2-hydroxy-3-(tetrahydrofuran-3-yl)-N-(6-(trifluoromethyl)pyridin-3-yl)benzamide (98)

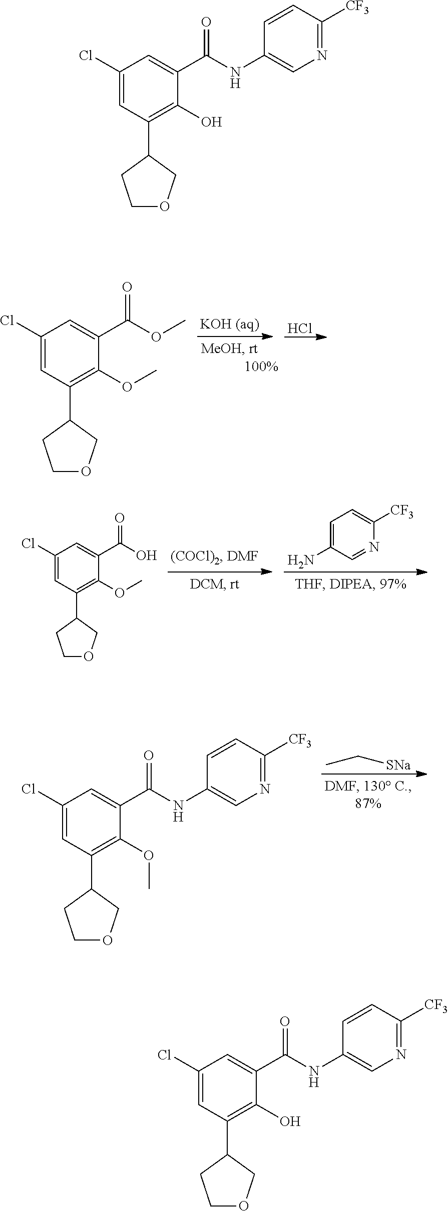

To a stirred solution of methyl 5-chloro-2-methoxy-3-(tetrahydrofuran-3-yl)benzoate (929 mg, 3.44 mmol, Example 14) in MeOH (10 mL) was added 10.0 mL 1N KOH solution. The resulting mixture was stirred at rt overnight. After completion of the reaction, the mixture was partitioned between 1N HCl and ethyl acetate. The organic layer was dried over sodium sulfate and concentrated under reduced pressure to afford a residue of 5-chloro-2-methoxy-3-(tetrahydrofuran-3-yl)benzoic acid. The residue was used directly in the next step.

5-chloro-2-methoxy-3-(tetrahydrofuran-3-yl)benzoic acid (81 mg, 0.316 mmol) was dissolved in DCM (5.0 mL), followed by the addition of a catalytic amount of DMF (1 drop) and oxalyl chloride (33 μL, 0.379 mmol) respectively. The reaction was allowed to stir at rt for 30 minutes then concentrated in vacuo. The residue was re-dissolved in THF (5.0 mL), and Hunig's base (66 μL, 0.379 mmol) and 6-(trifluoromethyl)pyridin-3-amine (51 mg, 0.316 mmol) were added. The mixture was stirred at rt for 48 hours before silica gel was added to quench the reaction. Solvent was evaporated and the resulting residue was purified via silica gel column chromatography to yield 5-chloro-2-methoxy-3-(tetrahydrofuran-3-yl)-N-(6-(trifluoromethyl)pyridin-3-yl)benzamide (124 mg, 97%) as a yellow solid. MS (ESI) [M+H]⁺ requires m/z 401.0, found m/z 401.1.

A solution of 5-chloro-2-methoxy-3-(tetrahydrofuran-3-yl)-N-(6-(trifluoromethyl)pyridin-3-yl)benzamide (124 mg, 0.31 mmol) in DMF (5 mL) was mixed with sodium ethanethiolate (130 mg, 1.55 mmol) and the resulting suspension was heated at 130° C. overnight. After completion of the reaction, 1N HCl was added to the reaction and extracted with ethyl acetate two times. The combined ethyl acetate layer was dried over sodium sulfate and concentrated under reduced pressure. The residue was purified via silica gel column chromatography to give 5-chloro-2-hydroxy-3-(tetrahydrofuran-3-yl)-N-(6-(trifluoromethyl)pyridin-3-yl)benzamide (103 mg, 87%) as a yellow solid. ¹H NMR (300 MHz, Chloroform-d) δ 11.73 (s, 1H), 9.09 (s, 1H), 8.84 (s, 1H), 8.47 (d, J=8.7 Hz, 1H), 7.84-7.57 (m, 2H), 7.36 (d, J=2.4 Hz, 1H), 4.24-4.04 (m, 2H), 4.01-3.83 (m, 2H), 3.84-3.71 (m, 1H), 2.53-2.35 (m, 1H), 2.14-1.96 (m, 1H). MS (ESI) [M−H]⁻ requires m/z 385.1, found m/z 385.3.

### Example 100

### 5-chloro-2-hydroxy-N-(4-(((4-hydroxybenzyl)oxy)methyl)-6-(trifluoromethyl)benzo[d]thiazol-2-yl)-3-methylbenzamide (100)

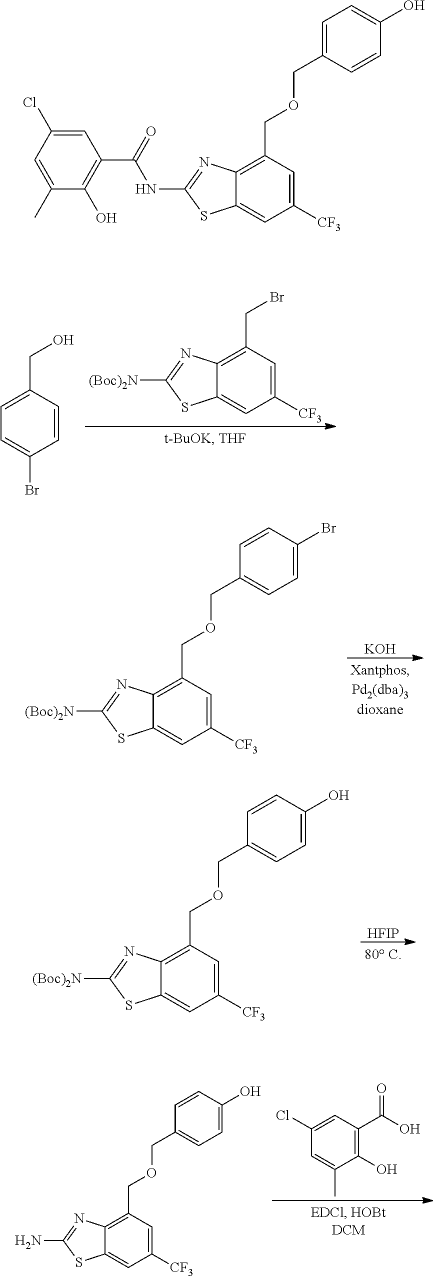

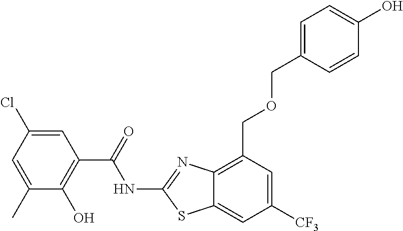 [SMILES: Cc1cc(Cl)cc(C(=O)Nc2nc3c(COCc4ccc(O)cc4)cc(C(F)(F)F)cc3s2)c1O]

To a solution of tert-butyl (4-(bromomethyl)-6-(trifluoromethyl)benzo[d]thiazol-2-yl)(tert-butoxycarbonyl)carbamate (546 mg, 1.07 mmol, Example 64) and (4-bromophenyl)methanol (200 mg, 1.07 mmol) in THE (10 mL) was added dropwise t-BuOK (1 M, 4.2 mL) at 0° C. under N₂. The resulting mixture was stirred at 0° C. for 1 hr. The reaction mixture was diluted with water (10 mL) and extracted with ethyl acetate (5 mL×3). The combined organic layer was washed with brine (10 mL×2), dried over Na₂SO₄, filtered and concentrated under reduced pressure to give a residue. The residue was purified by flash silica gel chromatography (ISCO®; 12 g SepaFlash® Silica Flash Column, Eluent of 0~5% ethyl acetate/petroleum ether gradient @70 mL/min) to afford compound tert-butyl (4-(((4-bromobenzyl)oxy)methyl)-6-(trifluoromethyl)benzo[d]thiazol-2-yl)(tert-butoxycarbonyl)carbamate (0.2 g, 168.43 μmol, 15.75% yield, 52% purity) as yellow oil.

tert-butyl (4-(((4-bromobenzyl)oxy)methyl)-6-(trifluoromethyl)benzo[d]thiazol-2-yl)(tert-butoxycarbonyl)carbamate (0.2 g, 323.90 μmol), KOH (363 mg, 6.48 mmol), Pd₂(dba)₃ (5 mg, 6.48 μmol) and di-tert-butyl-[2-(2,4,6-triisopropylphenyl)phenyl]phosphane (11 mg, 25.91 μmol) in dioxane (5 mL) and H₂O (5 mL) was degassed and purged with N₂ 3 times, and then the mixture was stirred at 110° C. for 2 h under N₂ atmosphere. The reaction mixture was concentrated under reduced pressure to remove solvent. The residue was diluted with water (5 mL) and extracted with ethyl acetate (5 mL×3). The combined organic layer was washed with brine (3 mL×3), dried over Na₂SO₄, filtered and concentrated under reduced pressure to give a residue. The residue was purified by prep-TLC (SiO₂, petroleum ether/ethyl acetate 3/1) to afford compound tert-butyl (tert-butoxycarbonyl)(4-(((4-hydroxybenzyl)oxy)methyl)-6-(trifluoromethyl)benzo[d]thiazol-2-yl)carbamate (0.1 g, 100.98 μmol, 31.18% yield, 56% purity) as yellow oil.

A solution tert-butyl (tert-butoxycarbonyl)(4-(((4-hydroxybenzyl)oxy)methyl)-6-(trifluoromethyl)benzo[d]thiazol-2-yl)carbamate (0.07 g, 126.22 μmol) in HFIP (3 mL) was stirred at 80° C. for 1.5 h. The reaction mixture was concentrated under reduced pressure to remove solvent. The residue was purified by prep-TLC (SiO₂, petroleum ether/ethyl acetate 1/1) to afford 4-(((2-amino-6-(trifluoromethyl)benzo[d]thiazol-4-yl)methoxy)methyl)phenol (17 mg, 26.72 μmol, 21.17% yield, 55.7% purity) as yellow oil.

A mixture of 4-(((2-amino-6-(trifluoromethyl)benzo[d]thiazol-4-yl)methoxy)methyl)phenol (25 mg, 70.55 μmol), 5-chloro-2-hydroxy-3-methylbenzoic acid (15 mg, 84.66 μmol) in DCM (3 mL) was added EDCI (16 mg, 84.66 μmol) and HOBt (14 mg, 105.83 μmol), and then the mixture was stirred at 45° C. for 12 hrs. The reaction mixture was concentrated under reduced pressure to remove solvent. The residue was purified by prep-HPLC (neutral condition) to afford 5-chloro-2-hydroxy-N-(4-(((4-hydroxybenzyl)oxy)methyl)-6-(trifluoromethyl)benzo[d]thiazol-2-yl)-3-methylbenzamide (7.54 mg, 14.31 μmol, 20.29% yield, 99.27% purity) as a white solid. ¹H NMR (400 MHz, DMSO-d₆) δ ppm 9.41 (br s, 1H), 8.44 (s, 1H), 8.02 (br s, 1H), 7.75 (s, 1H), 7.47 (br s, 1H) 7.19 (d, J=8.38 Hz, 2H), 6.74 (d, J=8.38 Hz, 2H), 4.93 (s, 2H), 4.55 (s, 2H), 2.16-2.27 (m, 3H). MS (ESI) [M+H]⁺ requires m/z 523.1, found m/z 522.9

### Example 101

### 5-chloro-2-hydroxy-N-(4-((2-methoxyacetamido)methyl)-6-(trifluoromethyl)benzo[d]thiazol-2-yl)-3-methylbenzamide (101)

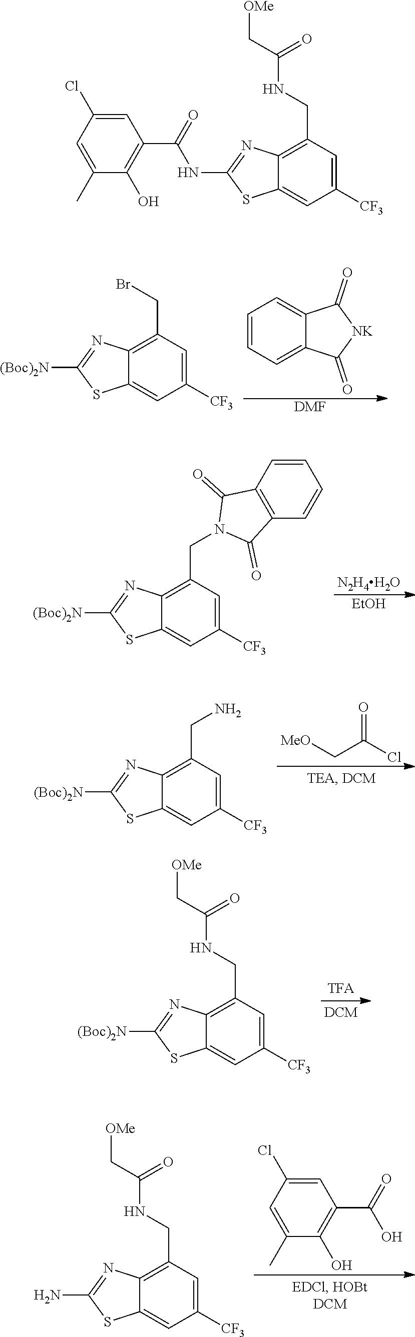

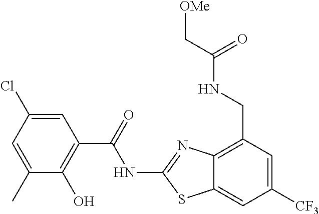 [SMILES: COCC(=O)NCc1cc(C(F)(F)F)cc2sc(NC(=O)c3cc(Cl)cc(C)c3O)nc12]

To a solution of tert-butyl (4-(bromomethyl)-6-(trifluoromethyl)benzo[d]thiazol-2-yl)(tert-butoxycarbonyl)carbamate (500 mg, 977.8 μmol, Example 64) in DMF (5 mL) was added potassium 1,3-dioxoisoindolin-2-ide (271 mg, 1.47 mmol). The mixture was stirred at 25° C. for 12 hrs. The reaction mixture was diluted with water (10 mL) and extracted with ethyl acetate (5 mL×3). The combined organic layers were washed with brine (15 mL×2), dried over Na₂SO₄, filtered and concentrated under reduced pressure to give a residue. The residue was purified by flash silica gel chromatography (ISCO®; 4 g SepaFlash® Silica Flash Column, Eluent of 0~10% ethyl acetate/petroleum ether gradient @70 mL/min) to afford tert-butyl (tert-butoxycarbonyl)(4-((1,3-dioxoisoindolin-2-yl)methyl)-6-(trifluoromethyl)benzo[d]thiazol-2-yl)carbamate (320 mg, 554.0 μmol, 56.6% yield) as a white solid.

To a suspension of tert-butyl (tert-butoxycarbonyl)(4-((1,3-dioxoisoindolin-2-yl)methyl)-6-(trifluoromethyl)benzo[d]thiazol-2-yl)carbamate (320 mg, 554.04 μmol) in EtOH (5 mL) was added N₂H₄·H₂O (56 mg, 1.1 mmol, 54.95 uL, 98% purity). The mixture was stirred at 25° C. for 5 h. The reaction mixture was concentrated under reduced pressure to remove solvent. The residue was diluted with DCM (20 mL), then filtered and concentrated under reduced pressure to afford tert-butyl (4-(aminomethyl)-6-(trifluoromethyl)benzo[d]thiazol-2-yl)(tert-butoxycarbonyl)carbamate (150 mg, crude) as a white solid.

To a solution of t tert-butyl (4-(aminomethyl)-6-(trifluoromethyl)benzo[d]thiazol-2-yl)(tert-butoxycarbonyl)carbamate (130 mg, 374.26 μmol) in DCM (3 mL) was added TEA (56 mg, 561.38 umol, 78 μL), then 2-methoxyacetyl chloride (48 mg, 449.11 μmol, 40 μL) was added at 0° C. The resulting mixture was stirred at 20° C. for 12 hrs. The reaction mixture was concentrated under reduced pressure to remove solvent. The residue was purified by prep-TLC (SiO₂, petroleum ether/ethyl acetate 1/1) to afford tert-butyl (tert-butoxycarbonyl)(4-((2-methoxyacetamido)methyl)-6-(trifluoromethyl)benzo[d]thiazol-2-yl)carbamate (140 mg, 298.41 μmol, 79.74% yield, 89.4% purity) as yellow oil.

To a solution of tert-butyl (tert-butoxycarbonyl)(4-((2-methoxyacetamido)methyl)-6-(trifluoromethyl)benzo[d]thiazol-2-yl)carbamate (140 mg, 333.80 μmol) in DCM (3 mL) was added TFA (1 mL). The mixture was stirred at 25° C. for 26 h. The reaction mixture was concentrated under reduced pressure to remove solvent. The residue was diluted with sat NaHCO₃ (20 mL) and extracted with ethyl acetate (3 mL×3). The combined organic layer was washed with brine (10 mL×2), dried over Na₂SO₄, filtered and concentrated under reduced pressure to give a residue. The residue was purified by prep-TLC (SiO₂, petroleum ether/ethyl acetate 1/1) to afford compound N-((2-amino-6-(trifluoromethyl)benzo[d]thiazol-4-yl)methyl)-2-methoxyacetamide (0.1 g, 295.77 μmol, 88.61% yield, 94.44% purity) as a white solid.

To a solution of N-((2-amino-6-(trifluoromethyl)benzo[d]thiazol-4-yl)methyl)-2-methoxyacetamide (0.08 g, 250.55 umol), 5-chloro-2-hydroxy-3-methylbenzoic acid (46 mg, 250.55 μmol) in DCM (3 mL) was added EDCI (57 mg, 300.66 μmol) and HOBt (50 mg, 375.82 μmol). The mixture was stirred at 50° C. for 12 hrs. The reaction mixture was concentrated under reduced pressure to remove solvent. The residue was purified by prep-HPLC (HCl condition) to afford 5-chloro-2-hydroxy-N-(4-((2-methoxyacetamido)methyl)-6-(trifluoromethyl)benzo[d]thiazol-2-yl)-3-methylbenzamide (21.24 mg, 43.54 μmol, 17.38% yield, 100% purity) as a white solid. 1H NMR (400 MHz, DMSO-d6) δ ppm 8.53-8.60 (m, 1H), 8.41 (s, 1H), 8.02 (br s, 1H), 7.56 (br s, 1H), 7.47 (br s, 1H), 4.74 (br s, 2H), 3.92 (s, 2H), 3.34 (s, 3H), 2.21 (s, 3H) MS (ESI) [M+H]+ requires m/z 488.1, found m/z 487.9

### Example 102

### 5-chloro-3-((cis-2,6-dimethylmorpholino)methyl)-2-hydroxy-N-(4-(methoxymethyl)-6-(trifluoromethyl)benzo[d]thiazol-2-yl)benzamide (102)

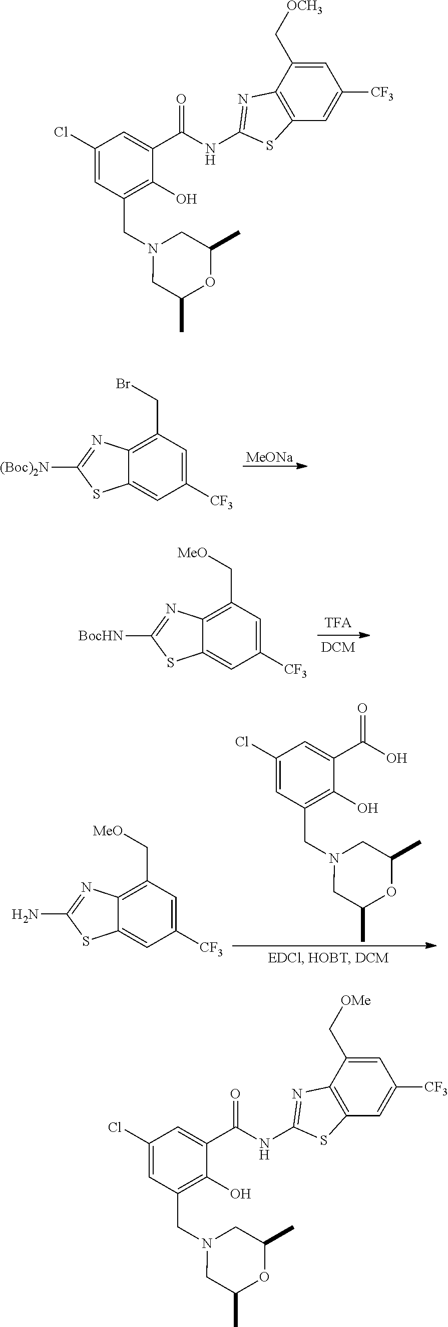

A solution of tert-butyl (4-(bromomethyl)-6-(trifluoromethyl)benzo[d]thiazol-2-yl)(tert-butoxycarbonyl)carbamate (0.4 g, 782.24 μmol, Example 64) in NaOMe (10 mL, 30% in MeOH) was stirred at 30° C. for 5 h. The reaction mixture was diluted with water (30 mL) and extracted with ethyl acetate (5 mL×3). The combined organic layer was washed with brine (30 mL), dried over Na₂SO₄, filtered and concentrated under reduced pressure to afford tert-butyl (4-(methoxymethyl)-6-(trifluoromethyl)benzo[d]thiazol-2-yl)carbamate (0.25 g, crude) as a yellow oil.

To a solution of tert-butyl (4-(methoxymethyl)-6-(trifluoromethyl)benzo[d]thiazol-2-yl)carbamate (0.25 g, 689.91 μmol) in DCM (3 mL) was added TFA (1 mL). The mixture was stirred at 20° C. for 1 hr. The reaction mixture was concentrated under reduced pressure to remove solvent. The residue was diluted with sat NaHCO₃ (10 mL) and extracted with ethyl acetate (5 mL×3). The combined organic layer was washed with brine (15 mL×3), dried over Na₂SO₄, filtered and concentrated under reduced pressure to give a residue. The residue was purified by prep-TLC (SiO₂, petroleum ether/ethyl acetate 1/1) to afford 4-(methoxymethyl)-6-(trifluoromethyl)benzo[d]thiazol-2-amine (136 mg, 475.03 μmol, 68.85% yield, 91.6% purity) as a yellow solid.

To a solution of 4-(methoxymethyl)-6-(trifluoromethyl)benzo[d]thiazol-2-amine (60 mg, 228.79 μmol), 5-chloro-3-[(cis,2,6-dimethylmorpholin-4-yl)methyl]-2-hydroxy-benzoic acid (82 mg, 274.55 μmol) in DCM (4 mL) was added EDCI (52 mg, 274.55 μmol) and HOBt (46 mg, 343 μmol). The mixture was stirred at 45° C. for 12 h. The reaction mixture was concentrated under reduced pressure to remove solvent. The residue was purified by prep-HPLC (neutral condition) to afford compound 5-chloro-3-((cis-2,6-dimethylmorpholino)methyl)-2-hydroxy-N-(4-(methoxymethyl)-6-(trifluoromethyl)benzo[d]thiazol-2-yl)benzamide (23.67 mg, 42.37 μmol, 18.52% yield, 97.373% purity) as a white solid. ¹H NMR (400 MHz, DMSO-d₆) δ ppm 8.35 (s, 1H), 7.81 (d, J=2.87 Hz, 1H), 7.64 (s, 1H), 7.38 (d, J=3.09 Hz, 1H), 4.84 (s, 2H), 4.22 (br s, 2H), 3.91 (br dd, J=9.37, 6.28 Hz, 2H), 3.42 (s, 3H), 3.29-3.36 (m, 4H), 1.13 (d, J=6.17 Hz, 6H). MS (ESI) [M+H]⁺ requires m/z 544.1, found m/z 544.1

### Example 103

### 5-chloro-3-((cis-2,6-dimethylmorpholino)methyl)-N-(4-(ethoxymethyl)-6-(trifluoromethyl)benzo[d]thiazol-2-yl)-2-hydroxybenzamide (103)

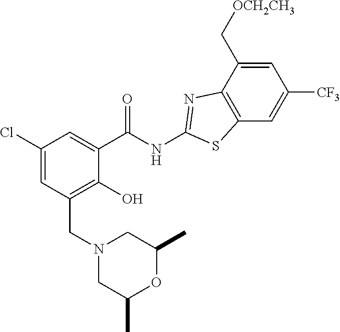 [SMILES: CCOCc1cc(C(F)(F)F)cc2sc(NC(=O)c3cc(Cl)cc(CN4C[C@@H](C)O[C@@H](C)C4)c3O)nc12]

5-chloro-3-((cis-2,6-dimethylmorpholino)methyl)-N-(4-(ethoxymethyl)-6-(trifluoromethyl)benzo[d]thiazol-2-yl)-2-hydroxybenzamide was prepared in a manner similar to Example 102.

LCMS: MS (ESI) [M+H]⁺ requires m/z 558.1, found m/z 558.1¹H NMR (400 MHz, DMSO-d₆) δ ppm 8.37 (s, 1H), 7.90 (br s, 1H), 7.68 (s, 1H), 7.48 (br s, 1H), 4.89 (s, 2H), 4.26 (s, 2H), 3.91 (br dd, J=10.03, 6.06 Hz, 2H), 3.62 (q, J=6.98 Hz, 2H), 3.34 (br d, J=12.13 Hz, 2H), 2.69 (br t, J=11.36 Hz, 2H), 1.21 (t, J=7.06 Hz, 3H), 1.13 (d, J=6.39 Hz, 6H

### Example 104

### 5-chloro-3-((cis-2,6-dimethylmorpholino)methyl)-N-(4-((2-methoxyethoxy)methyl)-6-(trifluoromethyl)benzo[d]thiazol-2-yl)-2-(((2S,3R,4S,5S,6R)-3,4,5-trihydroxy-6-(hydroxymethyl)tetrahydro-2H-pyran-2-yl)oxy)benzamide (104)

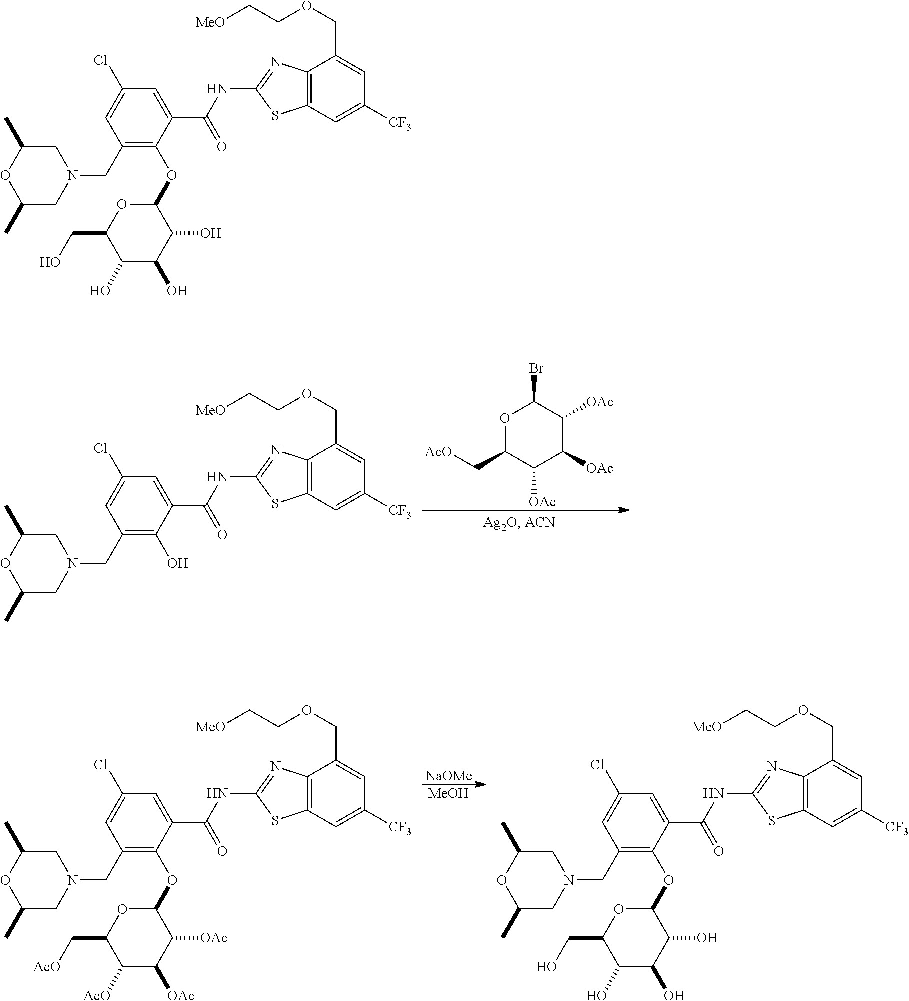

5-chloro-3-((cis-2,6-dimethylmorpholino)methyl)-2-hydroxy-N-(4-((2-methoxyethoxy)methyl)-6-(trifluoromethyl)benzo[d]thiazol-2-yl)benzamide (100 mg, 170.06 μmol, Example 64), (2R,3R,4S,5R,6S)-2-(acetoxymethyl)-6-bromotetrahydro-2H-pyran-3,4,5-triyl triacetate (139 mg, 340.11 μmol), Ag₂O (78 mg, 340.11 μmol) in ACN (9 mL), mixed then stirred at 30° C. for 12 h. The reaction mixture was concentrated under reduced pressure to remove solvent. The residue was purified by prep-TLC (SiO₂, petroleum ether/ethyl acetate 5/7) to afford (2R,3R,4S,5R,6S)-2-(acetoxymethyl)-6-(4-chloro-2-((cis-2,6-dimethylmorpholino)methyl)-6-((4-((2-methoxyethoxy)methyl)-6-(trifluoromethyl)benzo[d]thiazol-2-yl)carbamoyl)phenoxy)tetrahydro-2H-pyran-3,4,5-triyl triacetate (85 mg, 72.20 μmol, 42.45% yield, 78% purity) as yellow oil.

To (2R,3R,4S,5R,6S)-2-(acetoxymethyl)-6-(4-chloro-2-((cis-2,6-dimethylmorpholino)methyl)-6-((4-((2-methoxyethoxy)methyl)-6-(trifluoromethyl)benzo[d]thiazol-2-yl)carbamoyl)phenoxy)tetrahydro-2H-pyran-3,4,5-triyl triacetate (85 mg, 92.56 μmol) in MeOH (6 mL) was added NaOMe (33 mg, 185.12 μmol, 30% purity). The mixture was stirred at 25° C. for 5 h. The reaction mixture was concentrated under reduced pressure to remove solvent. The residue was purified by prep-HPLC (neutral condition) to afford 5-chloro-3-((cis-2,6-dimethylmorpholino)methyl)-N-(4-((2-methoxyethoxy)methyl)-6-(trifluoromethyl)benzo[d]thiazol-2-yl)-2-(((2S,3R,4S,5S,6R)-3,4,5-trihydroxy-6-(hydroxymethyl)tetrahydro-2H-pyran-2-yl)oxy)benzamide (3.72 mg, 4.82 μmol, 5.21% yield, 97.284% purity) as a white solid. ¹H NMR (400 MHz, DMSO-d₆) δ ppm 12.74 (br s, 1H), 8.44 (s, 1H), 7.72 (s, 1H), 7.61 (s, 1H), 7.56 (d, J=1.98 Hz, 1H), 5.42 (br s, 1H), 4.94 (s, 2H), 4.77 (d, J=5.29 Hz, 1H), 4.62 (d, J=7.72 Hz, 1H), 4.11-4.20 (m, 2H), 3.65-3.77 (m, 3H), 3.47-3.60 (m, 7H), 3.25 (s, 3H), 2.83 (br d, J=9.70 Hz, 1H), 2.65-2.69 (m, 1H), 1.65-1.82 (m, 2H), 1.03 (br t, J=7.06 Hz, 6H). MS (ESI) [M+H]⁺ requires m/z 750.2, found m/z 750.0

### Example 105

### 5-chloro-3-((trans-2,6-dimethylmorpholino)methyl)-2-hydroxy-N-(4-((2-methoxyethoxy)methyl)-6-(trifluoromethyl)benzo[d]thiazol-2-yl)benzamide (105)

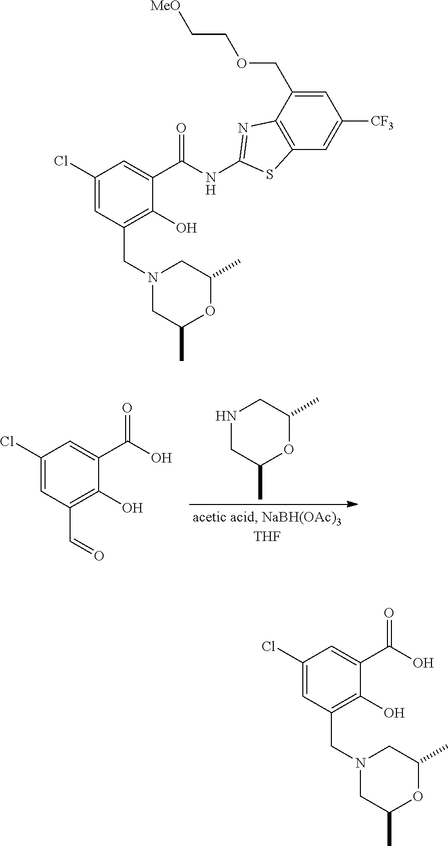

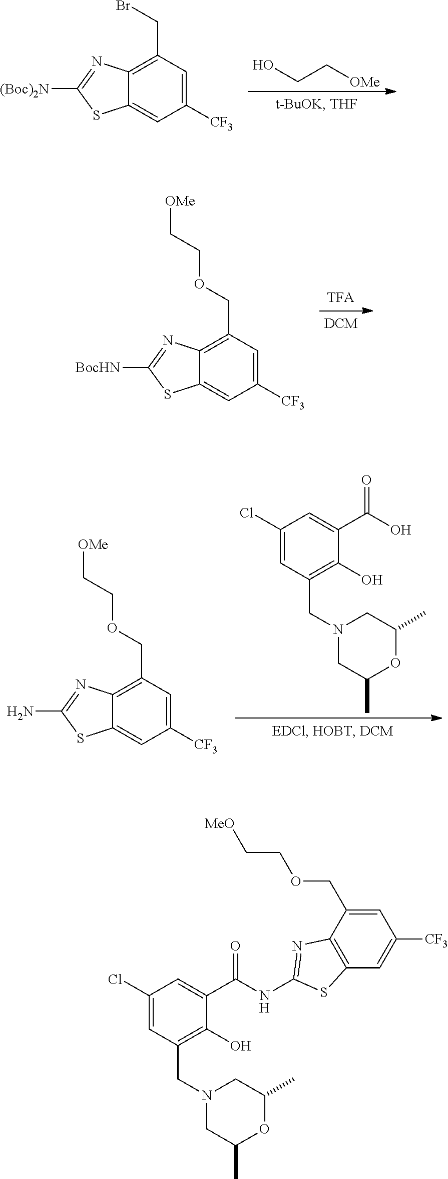

To a solution of 5-chloro-3-formyl-2-hydroxybenzoic acid (0.4 g, 1.99 mmol) and trans-2,6-dimethylmorpholine (459 mg, 3.99 mmol, 491 μL) in THE (30 mL) was added acetic acid (119 mg, 1.99 mmol, 114 μL) until the pH reached ~5-6. The mixture was stirred at 30° C. for 3 h then NaBH(OAc)₃ (2.5 g, 11.97 mmol) was added. The mixture was stirred at 30° C. for another 3 h. The reaction mixture was concentrated under reduced pressure to remove solvent. The residue was purified by prep-HPLC (neutral condition) to afford 5-chloro-3-((trans-2,6-dimethylmorpholino)methyl)-2-hydroxybenzoic acid (250 mg, 748.96 μmol, 37.56% yield, 89.8% purity) as a white solid.

To a solution of tert-butyl (4-(bromomethyl)-6-(trifluoromethyl)benzo[d]thiazol-2-yl)(tert-butoxycarbonyl)carbamate (400 mg, 586.68 μmol), 2-methoxyethanol (89 mg, 1.17 mmol, 92 μL) in THE (10 mL) was added t-BuOK (1M, 2 mL) at 0° C. under N₂ atmosphere. The suspension was degassed and purged with N₂ 3 times, then the mixture was stirred at 20° C. for 12 h. The reaction mixture was diluted with water (30 mL) and extracted with ethyl acetate (20 mL×3). The combined organic layer was washed with brine (100 mL), dried over Na₂SO₄, filtered and concentrated under reduced pressure to afford tert-butyl (4-((2-methoxyethoxy)methyl)-6-(trifluoromethyl)benzo[d]thiazol-2-yl)carbamate (230 mg, crude) as yellow oil.

To a solution of tert-butyl (4-((2-methoxyethoxy)methyl)-6-(trifluoromethyl)benzo[d]thiazol-2-yl)carbamate (230 mg, 565.92 μmol) in DCM (7 mL) was added TFA (1 mL). The mixture was stirred at 20° C. for 12 h. The reaction mixture was concentrated under reduced pressure to remove solvent. The residue was diluted with NaHCO₃ (20 mL) and extracted with ethyl acetate (4 mL×3). The combined organic layer was washed with brine (10 mL×3), dried over Na₂SO₄, filtered and concentrated under reduced pressure to give a residue. The residue was purified by prep-TLC (SiO₂, petroleum ether/ethyl acetate 1/1) to afford 4-((2-methoxyethoxy)methyl)-6-(trifluoromethyl)benzo[d]thiazol-2-amine (160 mg, 411.10 μmol, 72.64% yield, 78.7% purity) as a yellow solid.

To a solution of 5-chloro-3-((trans-2,6-dimethylmorpholino)methyl)-2-hydroxybenzoic acid (70 mg, 235.06 μmol), 4-((2-methoxyethoxy)methyl)-6-(trifluoromethyl)benzo[d]thiazol-2-amine (60 mg, 195.88 μmol) in DCM (4 mL) was added EDCI (45 mg, 235.06 μmol) and HOBt (39 mg, 293.83 μmol). The mixture was stirred at 45° C. for 12 h. The reaction mixture was concentrated under reduced pressure to remove solvent. The residue was purified by prep-HPLC (HCl condition) to afford 5-chloro-3-((trans-2,6-dimethylmorpholino)methyl)-2-hydroxy-N-(4-((2-methoxyethoxy)methyl)-6-(trifluoromethyl)benzo[d]thiazol-2-yl)benzamide (52.88 mg, 80.76 μmol, 41.23% yield, 95.37% purity, HCl) as a white solid. ¹H NMR (400 MHz, DMSO-d₆) δ ppm 8.45 (s, 1H), 8.07 (br s, 1H), 7.70-7.81 (m, 2H), 4.95 (s, 2H), 4.31 (s, 2H), 4.17 (br s, 2H), 3.70 (dd, J=5.62, 3.67 Hz, 2H), 3.54 (dd, J=5.62, 3.67 Hz, 2H), 3.20-3.28 (m, 5H), 2.97 (br s, 2H), 1.24 (br s, 6H). MS (ESI) [M+H]⁺ requires m/z 588.2, found m/z 588.1

### Example 106

### 5-chloro-2-hydroxy-N-(4-((2-methoxyethoxy)methyl)-6-(trifluoromethyl)benzo[d]thiazol-2-yl)-3-(pyrrolidin-1-ylmethyl)benzamide (106)

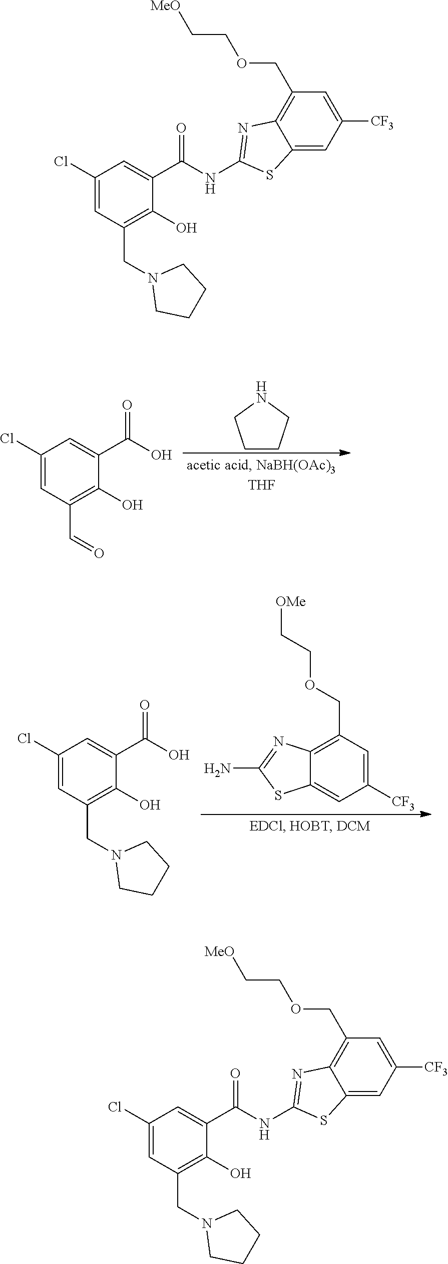 [SMILES: COCCOCc1cc(C(F)(F)F)cc2sc(NC(=O)c3cc(Cl)cc(CN4CCCC4)c3O)nc12 ; O=Cc1cc(Cl)cc(C(=O)O)c1O ; C1CCNC1 ; O=C(O)c1cc(Cl)cc(CN2CCCC2)c1O ; COCCOCc1cc(C(F)(F)F)cc2sc(N)nc12 ; COCCOCc1cc(C(F)(F)F)cc2sc(NC(=O)c3cc(Cl)cc(CN4CCCC4)c3O)nc12]

To a solution of 5-chloro-3-formyl-2-hydroxybenzoic acid (500 mg, 2.49 mmol) and pyrrolidine (195 mg, 2.74 mmol, 228 uL) in THF (15 mL) was added acetic acid (149 mg, 2.49 mmol, 142 uL) to reach a pH of ~5-6. The mixture was stirred at 30° C. for 3 h then NaBH(OAc)₃ (3.17 g, 14.96 mmol) was added. The mixture was stirred at 30° C. for another 3 h. The reaction mixture was concentrated under reduced pressure to remove solvent. The residue was purified by prep-HPLC (neutral condition) to afford 5-chloro-2-hydroxy-3-(pyrrolidin-1-ylmethyl)benzoic acid (420 mg, 1.60 mmol, 64.17% yield, 97.39% purity) as a yellow solid.

5-chloro-2-hydroxy-3-(pyrrolidin-1-ylmethyl)benzoic acid (40.07 mg, 156.71 μmol), 4-(2-methoxyethoxymethyl)-6-(trifluoromethyl)-1,3-benzothiazol-2-amine (40 mg, 130.59 umol) in DCM (4 mL) was added EDCI (30 mg, 156.71 μmol) and HOBt (26 mg, 195.88 μmol). The mixture was stirred at 45° C. for 12 h. The reaction mixture was concentrated under reduced pressure to remove solvent. The residue was purified by prep-HPLC (HCl condition) to afford 5-chloro-2-hydroxy-N-(4-((2-methoxyethoxy)methyl)-6-(trifluoromethyl)benzo[d]thiazol-2-yl)-3-(pyrrolidin-1-ylmethyl)benzamide (98.25% purity, HCl) 49.21 mg as a white solid. ¹H NMR (400 MHz, DMSO-d₆) δ ppm 10.00 (br s, 1H), 8.43 (s, 1H), 8.06 (br s, 1H), 7.78 (s, 1H), 7.70 (br s, 1H), 4.94 (s, 2H), 4.33 (br s, 2H), 3.67-3.71 (m, 2H), 3.51-3.57 (m, 6H), 3.27 (s, 3H), 1.97 (br s, 4H). MS (ESI) [M+H]⁺ requires m/z 544.1, found m/z 544.11

### Example 107

### 5-chloro-2-hydroxy-N-(4-(methoxymethyl)-6-(trifluoromethyl)benzo[d]thiazol-2-yl)-3-(pyrrolidin-1-ylmethyl)benzamide (107)

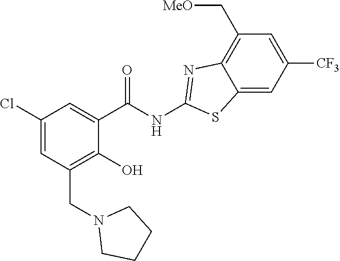 [SMILES: COCc1cc(C(F)(F)F)cc2sc(NC(=O)c3cc(Cl)cc(CN4CCCC4)c3O)nc12]

5-chloro-2-hydroxy-N-(4-(methoxymethyl)-6-(trifluoromethyl)benzo[d]thiazol-2-yl)-3-(pyrrolidin-1-ylmethyl)benzamide was prepared in a manner similar to Example 106. LCMS: MS (ESI) [M+H]⁺ requires m/z 500.1, found m/z 500. ¹H NMR (400 MHz, DMSO-d₆) δ ppm 10.06 (br s, 1H), 8.44 (s, 1H), 8.06 (br s, 1H), 7.70-7.75 (m, 2H), 4.85 (s, 2H), 4.33 (s, 2H), 3.40 (s, 7H), 1.97 (br s, 4H)

### Example 108

### (4-chloro-2-((cis-2,6-dimethylmorpholino)methyl)-6-((4-((2-methoxyethoxy)methyl)-6-(trifluoromethyl)benzo[d]thiazol-2-yl)carbamoyl)phenoxy)methyl acetate (108)

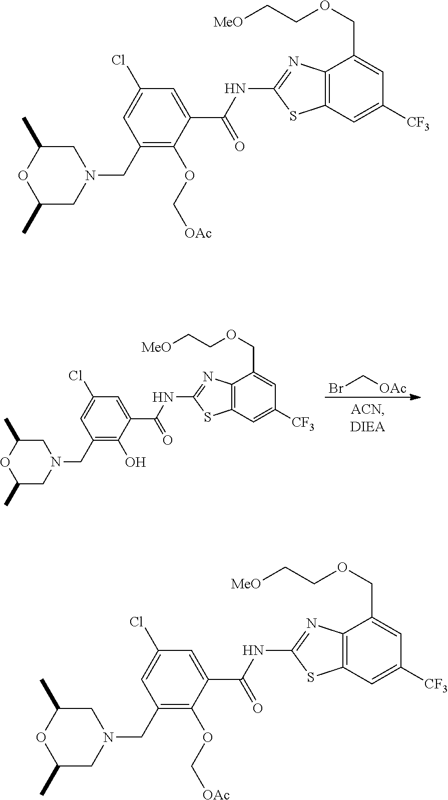 [SMILES: COCCOCc1cc(C(F)(F)F)cc2sc(NC(=O)c3cc(Cl)cc(CN4C[C@@H](C)O[C@@H](C)C4)c3OCOC(C)=O)nc12 ; COCCOCc1cc(C(F)(F)F)cc2sc(NC(=O)c3cc(Cl)cc(CN4C[C@@H](C)O[C@@H](C)C4)c3O)nc12 ; CC(=O)OCBr ; COCCOCc1cc(C(F)(F)F)cc2sc(NC(=O)c3cc(Cl)cc(CN4C[C@@H](C)O[C@@H](C)C4)c3OCOC(C)=O)nc12]

To a solution of 5-chloro-3-((cis-2,6-dimethylmorpholino)methyl)-2-hydroxy-N-(4-((2-methoxyethoxy)methyl)-6-(trifluoromethyl)benzo[d]thiazol-2-yl)benzamide (100 mg, 170.06 μmol, Example 64), DIPEA (65 mg, 510.17 μmol, 88 μL) in ACN (4 mL), was added bromomethyl acetate (78 mg, 510.17 μmol, 50 μL) slowly, and then the mixture was stirred at 20° C. for 12 h. The reaction mixture was concentrated under reduced pressure to remove solvent. The residue was purified by prep-HPLC (HCl condition) to afford (4-chloro-2-((cis-2,6-dimethylmorpholino)methyl)-6-((4-((2-methoxyethoxy)methyl)-6-(trifluoromethyl)benzo[d]thiazol-2-yl)carbamoyl)phenoxy)methyl acetate (10.41 mg, 14.46 μmol, 8.51% yield, 96.782% purity, HCl) as a white solid. ¹H NMR (400 MHz, DMSO-d₆) δ ppm 13.08 (s, 1H), 10.86 (br s, 1H), 8.51 (s, 1H), 8.10 (br s, 1H), 7.88 (br s, 1H), 7.78 (s, 1H), 5.55 (s, 2H), 4.96 (s, 2H), 4.37 (br s, 2H), 3.97 (br s, 2H), 3.69 (dd, J=5.62, 3.64 Hz, 2H), 3.52 (dd, J=5.51, 3.75 Hz, 2H), 3.26 (s, 3H), 2.78 (br d, J=8.82 Hz, 2H), 1.86-1.97 (m, 3H), 1.10-1.19 (m, 6H). MS (ESI) [M+H]⁺ requires m/z 660.2, found m/z 660.0

### Example B1

Determining Mitochondrial Uncoupling Activity by Oxygen Consumption Rate (OCR) Assay with Seahorse Instruments

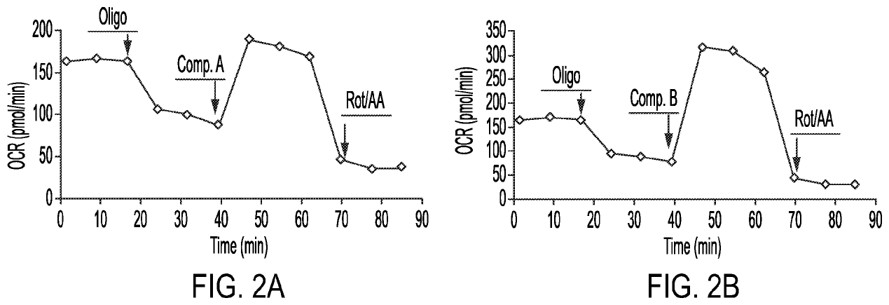

By definition, mitochondrial uncoupling is the decoupling of mitochondrial electron transport chain activity (mitochondrial oxidation) from ATP synthesis by mitochondrial ATP synthase. Technically, mitochondrial uncoupling activity is defined by the ability of a chemical compound to induce oxygen consumption of cells or isolated mitochondria in the presence of a mitochondrial ATP synthase inhibitor such as oligomycin. Accordingly, mitochondrial uncoupling activity was determined by oxygen consumption rate assay with a Seahorse XF96 or a Seahorse XF24 instrument following the vendor's instruction, using mouse myoblast C2C12 cells grown in a medium containing 10% fetal bovine serum. OCR was measured stepwise under the following conditions: first under normal growth conditions; then. OCR was measured after oligomycin was added to reach a final concentration of 2.5 μM); then OCR was measured after the compound was further added at varying concentrations. Finally, rotenone and antimycin A were added to confirm that the measurement was mitochondrial oxygen consumption (rotenone and a are mitochondrial electron transport chain inhibitors). FIG. 2 shows typical results demonstrating that the tested compounds 16 and 69, are mitochondrial uncouplers which increase OCR in the presence of oligomycin.

COCR-min the minimal concentration required for each compound to induce OCR in the presence of oligomycin, of disclosed compounds, was also determined by varying the final concentration of the compound added to the Seahorse OCR analysis experiment as described above (FIG. 2, varying testing compound concentration). Table 8 summarizes the mitochondrial uncoupling activity of disclosed compounds, as represented by COCR-min.

TABLE 8

| Compound | C_{OCR-min}¹ |
|---|---|
| 1 | *** |
| 2 | *** |
| 3 | *** |
| 4 | *** |
| 5 | ** |
| 6 | ** |
| 8 | *** |
| 9 | *** |
| 11 | *** |
| 13 | *** |
| 14 | *** |
| 15 | *** |
| 16 | *** |
| 17 | *** |
| 18 | *** |
| 19 | ** |
| 20 | ** |
| 21 | * |
| 22 | *** |
| 23 | *** |
| 24 | *** |
| 25 | *** |
| 26 | *** |
| 27 | *** |
| 28 | *** |
| 30 | * |
| 32 | *** |
| 33 | * |
| 36 | *** |
| 37 | * |
| 39 | * |
| 42 | ** |
| 43 | *** |
| 45 | ** |
| 46 | *** |
| 47 | *** |
| 49 | *** |
| 51 | *** |
| 53 | *** |
| 55 | *** |
| 56 | *** |
| 57 | *** |
| 58 | *** |
| 59 | *** |
| 60 | *** |
| 61 | *** |
| 62 | *** |
| 63 | *** |
| 64 | *** |
| 65 | *** |
| 66 | *** |
| 67 | *** |
| 68 | *** |
| 69 | *** |
| 70 | *** |
| 71 | *** |
| 72 | *** |
| 74 | *** |
| 75 | *** |
| 76 | *** |
| 78 | *** |
| 79 | *** |
| 82 | *** |
| 83 | *** |
| 84 | *** |
| 88 | ** |
| 89 | *** |
| 90 | ** |
| 91 | *** |
| 92 | *** |
| 93 | *** |
| 94 | *** |
| 95 | *** |
| 96 | *** |
| 97 | *** |
| 98 | * |

¹C_{OCR-min}: minimal concentration increasing OCR in the presence of oligomycin.
***, <5 μM;
** between 5 and 10 μM;
* >10 μM.

### Example B2

### Unique Property: Compounds Effectively Induce OCR Without Significant Loss of Mitochondrial Membrane Potential

As proton gradient is responsible for establishing mitochondrial membrane potential (MMP), the unregulated proton transport across mitochondrial inner membrane catalyzed by mitochondrial uncouplers dissipates MMP. Prior to the present disclosure, all mitochondrial uncouplers (including DNP, herein referred to as conventional uncouplers) tested have properties that increase OCR and decreased MMP, and the concentrations for increasing OCR and dissipating MMP correlated. As such, the activity for decreasing MMP is considered a second hallmark of mitochondrial uncoupling.

Mitochondrial membrane potential assay was performed using the standard TMRE (tetramethylrhodamine ethyl ester) staining method with cultured mammalian cells, the NIH-3T3 cells, C2C12 cells, and other mammalian cell lines. Cells were seeded in 6-well plates and cultured in DMEM medium supplemented with 10% fetal bovine serum and 2 mM glutamine. Cells were allowed to grow to the logarithmic growth phase prior to experiments. The cells were treated with each individual compound at various concentrations for two hours, followed by staining with TMRE at a final concentration of 100 nM for 15 minutes. The cells were then washed once with PBS, and examined under fluorescence microscopy. Cells treated with various concentrations of niclosamide ethanolamine were used as a positive control.

In some examples, mitochondrial membrane potential was measured with DilC1(5) staining. Briefly, DilC1(5) was added to the cells to 50 nM final concentration and the cells were incubated at 37° C., 5% CO₂, for 15 to 30 minutes. Cells were washed once by adding 2 mL of warm phosphate-buffered saline (PBS). Mitochondrial staining was determined by fluorescence microscopy with 633 nm excitation using standard filters for far-red emission.

As illustrated in FIG. 4, the conventional mitochondrial uncoupler FCCP starts to increase OCR and reduce MMP at 3 uM at the same time; at the concentration of 9 uM, MMP is totally lost and the rapid MMP loss correlates with the increase in OCR. This correlates with what is typically seen in conventional mitochondrial uncouplers. The window between the concentration showing minimal activity C_{min-OCR} and the concentration of conventional uncoupler dissipating MMP (losing 90% of MMP, defined as C_{10% TMRE}) is narrow, with the ratio of C_{10% TMRE}/C_{min-OCR} around 3 (FIGS. 4A and B).

Surprisingly, many of the compounds of the invention exhibited a unique feature shown in FIG. 5. Compound 25 started to increase OCR at around 1.0 μM, and showed very robust OCR induction at 3.0, 6.0, and 9.0 μM (FIG. 5A). However, Compound 25 does not cause observable MMP reduction under these conditions (FIG. 5B).

In addition, another group of MMP-retaining uncouplers were identified, as represented by Compound 64. Unlike Compound 25, which does not lead to any observable loss in MMP, Compound 64 appears to lead to a minor loss of MMP at the highest level of mitochondrial uncoupling (highest OCR), as illustrated in FIG. 6. But the MMP loss is drastically lower than that induced by a conventional uncoupler such as FCCP (FIG. 4).

Using the ratio between C_{10% TMRE} (the concentration at which 10% MMP remains, i.e. 90% MMP is lost) and C_{min-OCR} (the concentration at which minimal OCR induction is observed), we categorized the MMP-retaining uncouplers into two groups. Compound 25-like compounds are denoted as MMP-Retaining Uncoupling Compounds (C_{10% TMRE}/C_{min-OCR}>25). The Compound 64-like compounds are denoted as MMP-Retaining Uncoupling Compounds (C_{10% TMRE}/C_{min-OCR} between 10 to 25). The conventional uncouplers, including FCCP and DNP, have a C_{10% TMRE}/C_{min-OCR} that is equal to or less than 3. The compounds are summarized in Table 6.

### Example B3

New Benzamide Mitochondrial Uncouplers with Drastically Improved PK profiles

For PO pharmacokinetic studies, the compound was suspended in 0.5% CMC-Na+1.0% Tween 80 in water at 1.0 mg/ml, dosing at 10 mg/kg by gavage. The blood concentration of the compound was then determined at 0 (pre-dosing), 5 min, 15 min, 30 min, 1 hr, 2 hr, 4 hr, 8 hr, 24 hr time points by LC-MS/MS analysis (See Tables 1 and 2, vide supra).

### Example B4

Efficacy on Type 2 Diabetes and Fatty Liver In Vivo: the Effect on Blood Glucose, Blood Insulin, and Fatty Liver in High-Fat Diet (HFD)-Induced Diabetic Mouse Model and db/db Mouse Model.

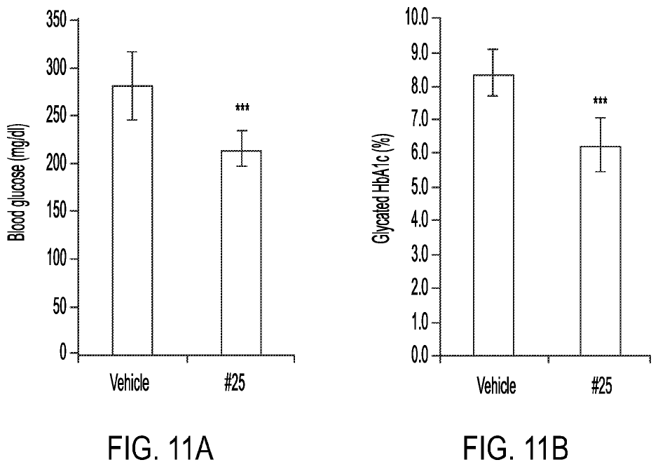

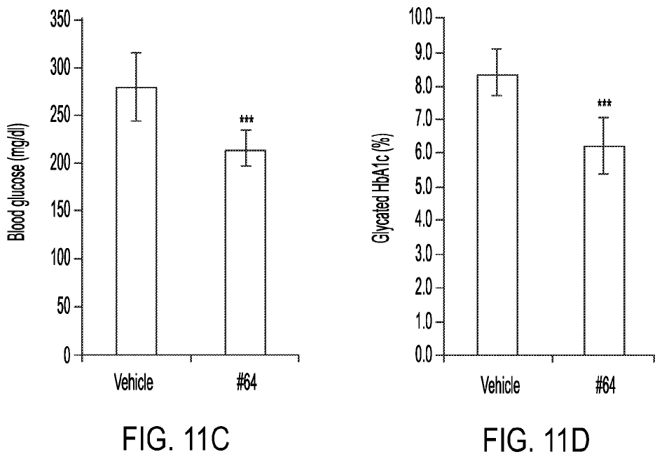

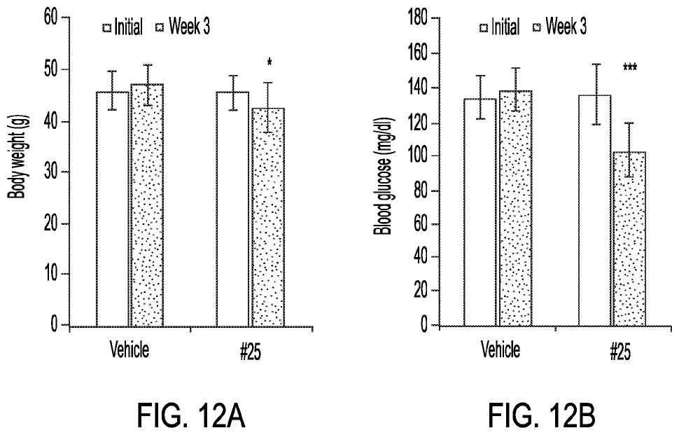

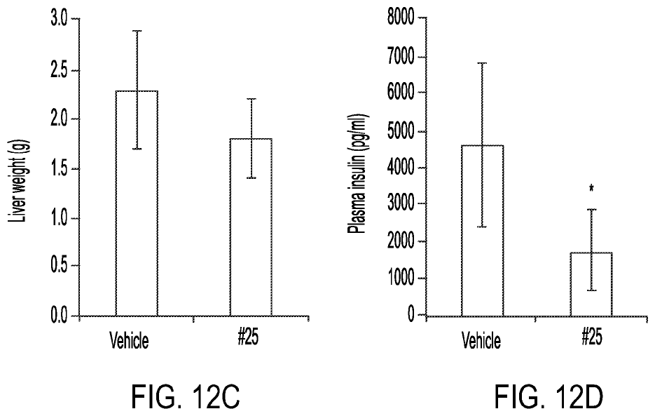

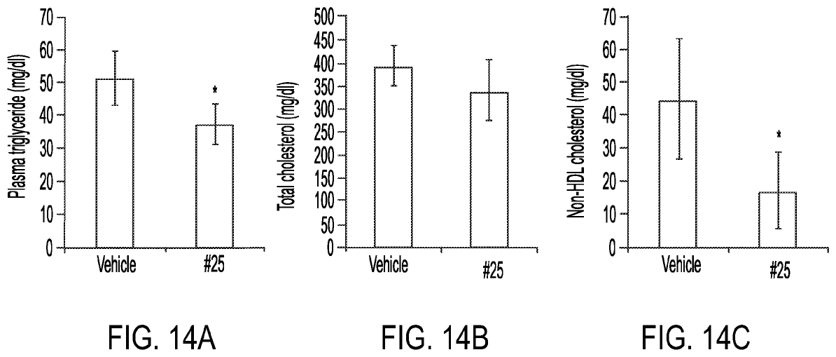

To demonstrate the efficacy of compounds of the invention in treating type 2 diabetes and steatosis in vivo, Compound 25 (FIGS. 11-15) and other compounds (Table 9) were tested in clinically relevant mouse models to show the effect on blood glucose, blood insulin concentration, liver steatosis, as well as other parameters such as blood lipid and cholesterol. FIG. 11 shows results from db/db mice, and FIG. 12-15, show results from HFD induced diabetic model. For HFD induced diabetic mouse model, C57Bl6 mice (male, n=6) were induced to obese and diabetic conditions by feeding high-fat diet (HFD, 60% fat calories, Research Diet, Inc. New Brunswick, NJ) for 3-4 months. The mice were then treated with P.O. dosing (gavage) of 5 mg/kg Compound 25 or Compound 64 once daily, or vehicle, for 3 weeks. During the 3-week period, the mice continued feeding HFD. The db/db (BKS.CgDock7m+/+Lepr db/J; stock #000642) mice (male, n=6) purchased from the Jackson Laboratory (Bar Harbor, ME), were treated with P.O. dosing (gavage) of 5 mg/kg Compound 25 or Compound 64 once daily, or vehicle, for 3 weeks. The blood glucose levels were measured after an overnight fast, plasma insulin levels were tested after 5-6 hours fast, and the glycated HbA1c levels were determined in whole blood samples. The results (FIG. 11-FIG. 13) show Compound 25 and Compound 64 are highly efficacious in reducing blood glucose, blood insulin, and glycated HbA1c in the clinically relevant models.

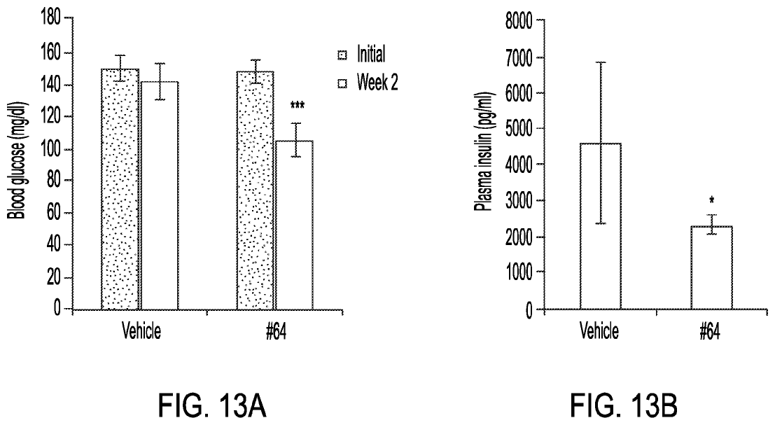

Compound 25 is efficacious in reducing blood triglyceride levels and non-HDL cholesterol levels induced by high-fat diet feeding (FIG. 13).

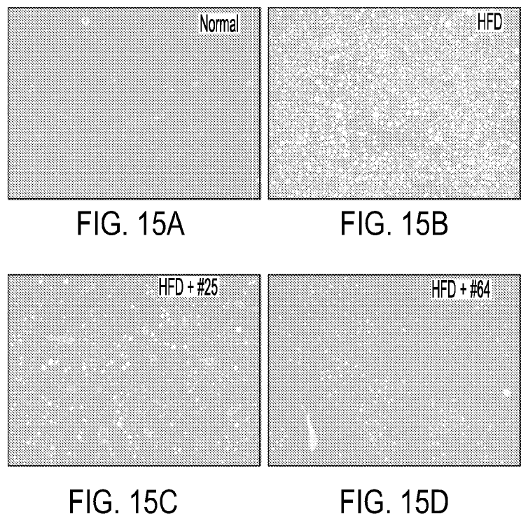

The effect of Compound 25 or 64 on liver steatosis was evaluated by direct comparison of liver histology (FIG. 15) between the drug-treated and vehicle-treated high-fat diet mice. The drug is highly efficacious in reversing fatty liver developed by HFD feeding. FIG. 15 shows liver histology images with H&E stain from mice treated as noted: FIG. 15A shows a normal liver section histology from a healthy C57Bl6 mouse without HFD feeding; FIG. 15B shows liver section histology of a mouse fed HFD. FIG. 15C and FIG. 15D show liver sections from mice fed HFD following 3 weeks of treatment with Compounds 25 and 64, respectively (P.O. dosing of 5 mg/kg, by daily gavage).

Experiments were similarly performed with other compounds, in either high-fat diet induced diabetes and fatty liver models, or in db/db mouse model, or in both, by daily oral gavage administration with low dosages. Table 9 shows the compounds, the animal model tested, and corresponding efficacious daily dose.

TABLE 9

| | Efficacious dosage to reduce blood |
|---|---|
| Com- | glucose in mouse models, |
| pound | oral daily gavage |
| 11 | 1.0 mg/kg (HFD model) |
| 15 | 2.5 mg/kg (HFD model) |
| 16 | 1.0 mg/kg (HFD model) |
| 25 | 2.5 mg/kg (both HFD and db/db models) |
| 23 | 10 mg/kg (HFD model) |
| 64 | 1.0-2.5 mg/kg (both HFD and db/db models) |

Collectively, the data show that Compounds 25 and 64, and the other compounds tested, at low dosages (1-10 mg/kg/day, by daily oral gavage administration), are highly efficacious in treating the above-mentioned symptoms in metabolic diseases in clinically relevant models. The efficacious dosages are greatly reduced as compared to prior disclosed compounds and the compounds do not require that the drug be mixed in food as compared to the prior disclosed compounds (International Patent Publication Number WO 2012/068274, International Patent Publication Number WO 2016/081599, U.S. Pat. No. 10,227,315, and Tao et al., 2014).

### Example B5

### Inhibiting Hepatic Fibrosis In Vitro and In Vivo

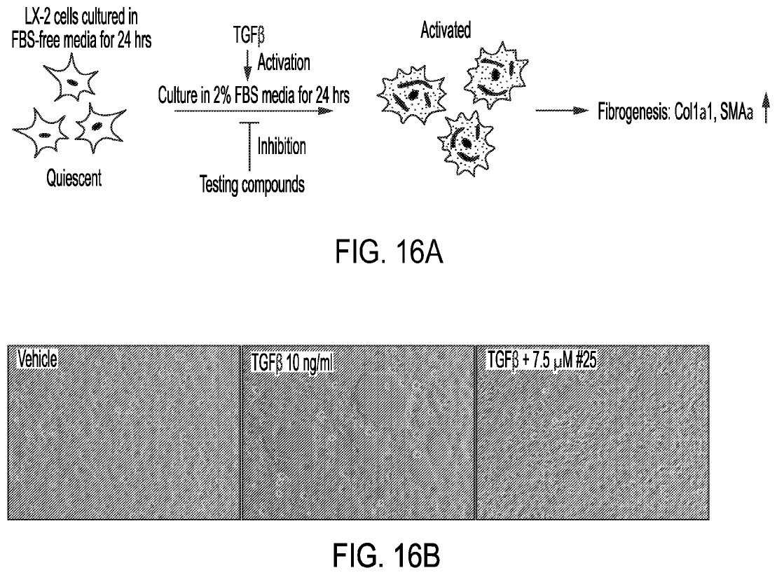

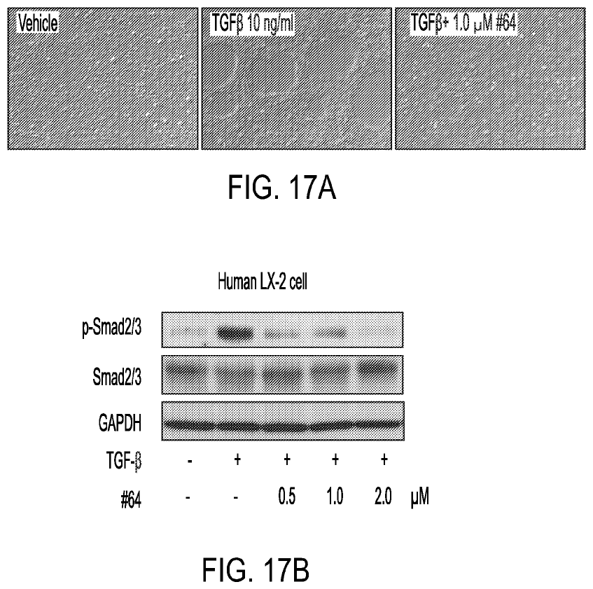

Liver fibrosis is one hallmark of NASH. Liver fibrosis is mainly caused by hepatic stellate cells differentiating into myofibroblast cells upon stimulation by various inflammatory signals in response to liver damage, induced by steatosis and other factors. This process could be modeled in vitro using the hepatic stellate differentiation experiment. The human hepatic stellate LX-2 cells were cultured within DMEM (Dulbecco's Modified Eagle Media, pH 7.4) supplied with 2% fetal bovine serum, 4.5 g/l glucose and 2 mM glutamine to 100 percent confluence. TGFβ (10 ng/ml) and Compound 25 (7.5 μM) or Compound 64 (1.0 μM) were then added into the media and incubated for 24 hours. Cellular morphology was then examined under a microscope. As shown in FIG. 16 to FIG. 17, hepatic stellate cells could be induced by TGF-beta to differentiate into myofibroblast-like cells. The experiments showed that treatment with Compound 25 and 64 effectively inhibit the differentiation process, indicating these compounds are efficacious in blocking stellate cell differentiation. FIG. 16B shows a panel of microscopic images of human hepatic stellate LX-2 cells in vehicle (DMSO), treated with TGFβ which induces the differentiation of LX-2 cells into myofibroblast-like cells, which form the “ring”-shape morphology after TGFβ treatment, and treated with TGFβ and Compound 25 which prevents TGFβ-induced differentiation of LX-2 cells. FIG. 17A shows a panel of microscopic images of human hepatic stellate LX-2 cells treated with TGFβ and Compound 64 which prevents TGFβ-induced differentiation of LX-2 cells.

Some other example compounds tested and their effectiveness of inhibiting hepatic stellate cells differentiating to myofibroblast-like cells are summarized in Table 10.

TABLE 10

| | Minimal inhibitory |
|---|---|
| Compounds | concentration |
| 64 | 1.0 μM |
| 25 | 7.5 μM |
| 16 | 5.0 μM |
| 23 | 5.0 μM |

The molecular mechanism by which Compound 64 inhibits TGF-β signaling pathway was examined. Immunoblotting analysis showed Compound 64 blocks activation of TGF-β signaling pathway at early stages by inhibiting Smad2/3 phosphorylation (FIG. 17B). LX-2 cells were treated with either vehicle (control), or TGF-β alone, or TGF-β plus indicated concentrations of Compound 64 for 6 hrs. Cells were then harvested, and immunoblotting analyses was performed with antibodies against p-Smad2/3 (phosphorylated Smad2/3), Smad2/3, or GAPDH, as indicated.

Since Compounds 25, 64, 16, and 23, were able to directly inhibit hepatic stellate cell (HSC) activation in vitro, a direct in vivo anti-fibrotic effect test (independent of anti-steatosis effect) was conducted using the carbon tetrachloride (CCl₄)-induced liver-damaging and liver fibrosis NASH murine models. CCl₄ is a hepatotoxin that induces acute liver injury in mice and induces oxidative damage, inflammation, and liver fibrosis in mice, independent of liver steatosis, and thus is considered as a “pure” fibrotic model.

C57Bl6 male mice at the age of 7-8 weeks were randomized into 3 groups: vehicle, CCl₄ alone treatment, CCl₄ plus Compound treatment, n=7. CCl₄ was diluted in olive oil (1:5 v/v), and intraperitoneally injected at a dose of 750 μl/kg to the mice, twice a week for 6 weeks. Compound treatment started at the beginning of CCl₄ injection. At the end of treatment, the mice were euthanized and liver tissue was collected. For histology analyses, the liver tissue was fixed and paraffin embedded; tissue sections were stained with hematoxylin and eosin (H&E), or with Picrosirius red. The fibrosis severities were scored by a board-certified pathologist based on Picrosirius red stain of the liver. For immunoblotting analyses, the liver tissue was homogenized and the proteins were separated by native page gel electrophoresis. The levels of Collagen I and GAPDH were detected by Collagen I polyclonal antibody (Abcam ab34710) and GAPDH (14C10) Rabbit mAb (Cell signaling #2118).

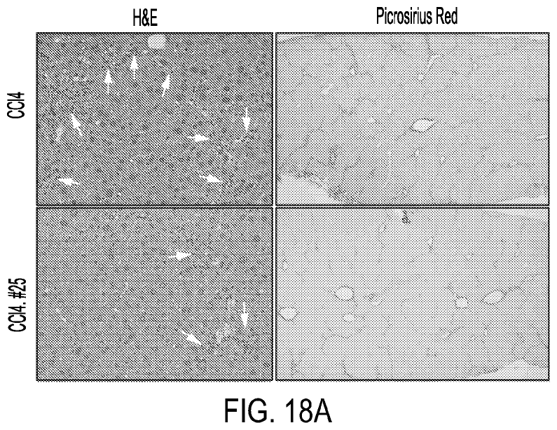

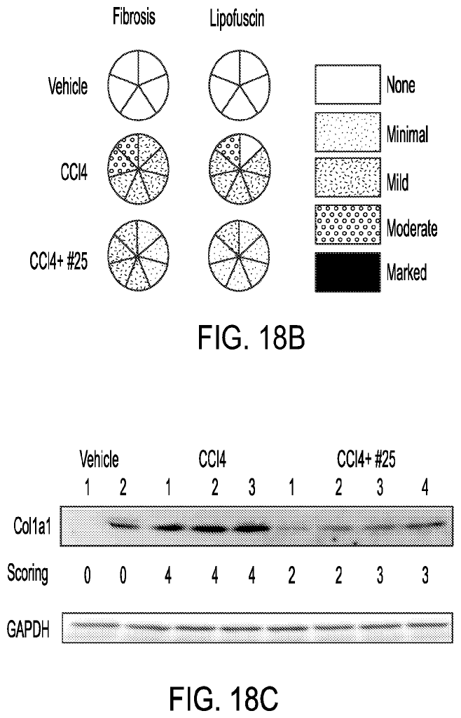

As shown in FIG. 18, treatment with Compound 25 reduced fibrosis formation and improved fibrosis scores (FIGS. 18A-B). Moreover, molecular characterization by immunoblotting with collagen antibody confirmed that Compound 25 reduces collagen levels, the biomarker of fibrosis (FIG. 18C).

### Example B6

### Effect on Cancer Cell Growth Inhibition

Cell growth inhibition assays were performed using a protocol standardized by National Cancer Institute (NCI) for evaluating anti-cancer drugs. Various mammalian cell lines were used for the assays. Briefly, the cells were inoculated into 96 well microtiter plates. After cell inoculation, the microtiter plates were incubated for cell growth for 24 h prior to the addition of compounds. After 24 h, two plates of each cell line were fixed in situ with TCA, to represent a measurement of the cell population for each cell line at the time of compound addition (Tz). Test compounds were solubilized in dimethyl sulfoxide. At the time of test compound addition, five 10-fold or ½ log serial dilutions were made to provide a total of five test compound concentrations plus control. Aliquots of 100 μl of these different test compound dilutions were added to the appropriate microtiter wells already containing 100 μl of the medium, resulting in the required final test compound concentrations. Following test compound addition, the plates were incubated for an additional 48 h. The assay was terminated by the addition of cold TCA. Cells were fixed in situ with 50 μl of cold 50% (w/v) TCA (final concentration, 10% TCA), washed five times with tap water and air-dried. Sulforhodamine B (SRB) solution (100 μl) at 0.4% (w/v) in 1% acetic acid was added to each well, and plates were incubated for 10 minutes at room temperature. After staining, the unbound dye was removed by washing five times with 1% acetic acid and the plates were air-dried. The bound stain was subsequently solubilized with 10 mM trizma base, and the absorbance was read on an automated plate reader at a wavelength of 515 nm. Using the seven absorbance measurements [time zero, (Tz), control growth, (C), and test growth in the presence of a compound at the five concentration levels (Ti)], the percentage growth was calculated at each of the test compound concentrations levels. Growth inhibition of 50% (GI₅₀) was calculated from [(Ti−Tz)/(C−Tz)]×100=50, which is the test compound concentration resulting in a 50% reduction in the net protein increase (as measured by SRB staining) in control cells during the compound incubation. Table 11 shows the GI₅₀ of the tested example compounds.

TABLE 11

| Compound | GI₅₀ (μM) ¹ | Cell line |
|---|---|---|
| 11 | ** | C2C12 |
| 13 | ** | C2C12 |
| 25 | * | NIH3T3 |
| 71 | *** | HepG2 |
| 68 | *** | HepG2 |
| 57 | *** | HepG2 |

¹, GI₅₀, 50% growth inhibition activity:
***, <5 μM;
** ≥5 but ≤ 20 μM;
* >20 μM

### Example B7

### Metabolic Stability Assay

The metabolic stability of the compounds was evaluated in incubation with rat liver microsomes. Briefly, 1 μM of each testing compound was incubated within rat liver microsomes (from BD Gentest) with final liver microsomal protein concentration of 0.5 mg/ml for different time points (0, 5, 15, 30 and 45 min) at 37° C., in potassium phosphate buffer plus 6 mM NAPDH. After the incubation, the reactions were quenched and the remaining compound concentrations were then extracted and determined by LC-MS/MS.Results are described in Tables 4 and 5.

### Example B8

### Rat Pharmacokinetics Study

Pharmacokinetics of the testing compounds was determined in male SD rats, n=3. Briefly, each compound was prepared in 0.5% CMC-Na and 1% Tween in water to yield a homogeneous suspension at 1 mg/ml, and then administrated to 10 mg/kg via oral gavage. The animals were fasted overnight before dosing and fed for 4 hour post dosing with free access to water for all time. Blood samples were collected at pre-dose, 0.083, 0.25, 0.5, 1, 2, 4, 8 and 24 hours, into K₂EDTA-coated tube via tail vein for serial sampling and cardiac puncture for terminal sampling. Blood samples were put on ice and centrifuged to obtain plasma samples (2000 g, 5 min under 4° C.) within 15 minutes. Plasma samples were stored at approximately −80° C. until analysis. The compound in the plasma samples were then extracted and concentrations determined by LC-MS/MS.

### Example B9

### Determination of Acute Toxicity

Acute toxicity tests were performed on C57Bl6 mice, male, 6 weeks old, n=6 for each dose. Compounds 64 and 25 were prepared to fine suspension in 0.5% CMC-Na/1% Tween80 water solution. The tests were performed under fed condition, by oral gavage of 100-400 μL compound suspension according to the body weight to desired dosages (mg/kg). The mice were supplied with drinking water all through the testing time. Mouse behaviors were monitored every 15-30 minutes. The dosage leading to the death of 50% of animals is defined as LD50. In FIG. 8, LD50 of Compound 64 was measured at around 420 mg/kg, while the LD50 of Compound 25 was measured over 500 mg/kg.

### Example B10

### Short-Term Toxicology Study

10-day escalating toxicology study in CD-1 mice: Different doses of compounds (e.g. Compound 25 and Compound 64) were given to male CD1 mice once daily via oral gavage, for 10 consecutive days. The compound was administered as a homogeneous suspension in 0.5% CMC+1% Tween80 in water in a volume of 10 mL/kg for oral administration. There were two arms in the study: 5 mice in the toxicology group and 5 mice in the toxicokinetic (TK) satellite group. Clinical signs were recorded twice daily. Body weight and food consumption were recorded once daily. Rectal temperature was recorded once every two days for a total of 5 times throughout the study. Necropsies were conducted at the termination of the study. Hematology, serum chemistry, plasma exposure, and tissue distribution were evaluated post 10th dosing in the TK satellite animals. Histopathology was examined in selected organs/tissues. Based on the outcomes of the toxicology parameters, NOAEL (no-observable-adverse-effect-level) and MTD (maximal tolerable dose) were determined.

In FIG. 9 and FIG. 10, for Compound 25, 25 mg/kg/day, 50 mg/kg/day, and 75 mg/kg/day were tested; for Compound 64, 20 mg/kg/day, 30 mg/kg/day, and 40 mg/kg/day were tested. The NOAEL of Compound 25 was 50 mg/kg/day, while the NAOEL of Compound 64 was 40 mg/kg/day.

### Example B11

TGF-β Inhibitory Effect in T cells

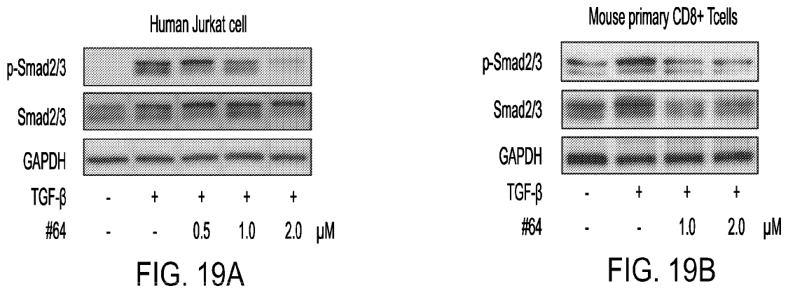

The effect of compounds (e.g. Compound 25 and Compound 64) on the TGF-β signaling pathway was analyzed in T-Cells. Jurkat cells (a human T-cell cancer cell line) and the primary mouse CD8+ cells (T-cells) were tested. The cells were treated with TGF-β (10 ng/ml) alone, or TGF-β (10 ng/ml) plus compound at various concentrations (e.g. 0.5, 1.0, 2.0 μM), or treated with vehicle (control), for 6 hours. The cells were then harvested, and cell lysates were subjected to immunoblotting analyses with antibodies against Smad2/3, phosphorylated Smad2/3, and GAPDH. As shown in FIG. 19, Compound 64 effectively blocks TGF-β signaling pathway activation in T cells, as demonstrated by inhibiting Smad2/3 phosphorylation, an essential event during TGF-β activation.

### Example B12

Anti-Cancer Effect in Combination with a PD-1 Inhibitor

Cancer immunotherapy provides a powerful therapeutic strategy. For example, antibodies against PD-1/PD-L1 and cell therapy using CAR-T have been proved efficacious in some cancers. However, many types of tumors do not respond to these immunoncology therapies. Moreover, within the same type of cancer, immunoncology therapies are not equally efficacious for all patients. One important variable that dictates the immunoncology treatment outcomes is the cancer microenvironment (CME). The various cancer metabolites in CME as well as the presence of high levels of TGF-β are known to inhibit T-cells which may otherwise be efficacious in eliminating cancer cells.

Due to the strong inhibitory effect of Compound 64 on the TGF-β pathway, a combinatory treatment of anti-PD1 antibody with Compound 64 was tested to determine the efficacy of the combination against metastatic liver cancer of various origins, such as colorectal origin.

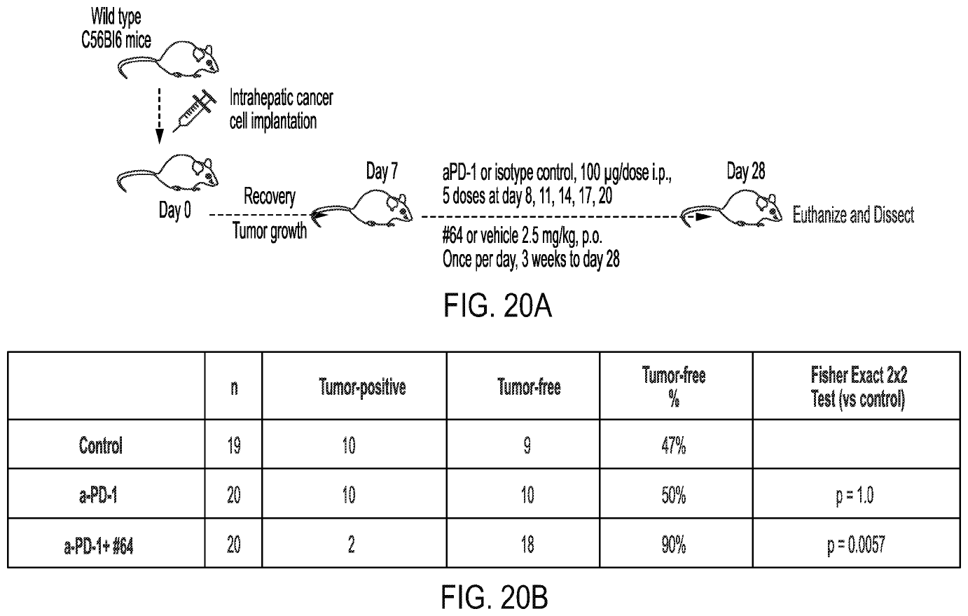

The experimental design is illustrated in FIG. 20A. Mouse colon cancer cells, MC38, were transplanted in livers of immune competent B57/Bl6 mice, and the effect of the combinatory treatment was determined. 100,000 or 250,000 MC38 cells suspended in 50 μl PBS were injected in the main lobe of the liver of C57Bl6 mice (female, 8 weeks old). 1 week after cancer cell transplantation, the mice were randomized in 3 groups for the following treatments: vehicle, anti-PD-1 antibody alone, and Compound 64 plus anti-PD-1 antibody. Compound 64 was prepared as fine suspension in 0.5% CMC-Na+1% Tween80 water solution. Treatment was started from day 7, at a dose of 2.5 mg/kg, once per day by oral gavage. The InVivoPlus Anti-mouse PD-1 (CD279) antibody, and its control antibody, InVivoPlus rat IgG2a isotype control (BioXcell), were used. The antibodies were diluted using dilution buffer from the BioXcell. The Anti-PD-1 antibody and its control were administrated to the mice as 100 μg per mouse via i.p. injection for a total of 5 doses on day 8, 11, 14, 17 and 20 after the cancer cell implantation. The mice were euthanized on day 28. Tumor incidence and volume were determined.

As shown in FIG. 20B, the combinatory treatment is highly efficacious as compared to vehicle or PD-1 antibody alone treated group, leading to the elimination of cancer in 90% of mice (90% cancer-free, P<0.01).

### Example B13

### Anti-Viral Activity Against Enveloped Viruses, Using Coronavirus 229E and SARS-CoV-2 as Examples

Replication and release of enveloped viruses require the host cells to synthesize lipids that support the assembly of viral envelopes, which consist of lipid bilayer. Mitochondrial uncouplers promote lipid oxidation and inhibit lipid synthesis. Thus, the compounds of the present invention were tested for antiviral activities against envelope viruses, using coronavirus 229E and SARS-CoV-2 (the pathogen of COVID19) as examples.

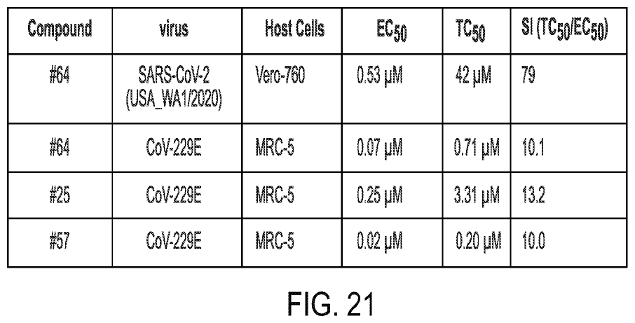

Compounds 64, 25, and 57, were tested, and specificity index (SI) was determined. Inhibition of virus-induced cytopathic effects (CPE) and cell viability following SARS-CoV-2 (USA WA1/2020 strain) replication or alpha coronavirus 229E replication in Vero 760 cells (SARS-COV-2) or MRC-5 cells (CoV-229E) was measured. Cells were seeded in 96-well flat-bottom tissue culture plates and allowed to adhere overnight. Following overnight incubation, diluted test compounds and virus diluted to a pre-determined titer to yield 85 to 95% cell killing at 3 days (SARS CoV-2) or 6 days (CoV-229E) post-infection were added to the plate. Following incubation at 37° C., 5% CO₂ for three or six days, cell viability was measured by Visual or sulforhodamine B staining. Percent CPE reduction of the virus-infected wells and the percent cell viability of uninfected drug control wells were calculated to determine the EC50 and TC50 values. Specificity Index (SI, ratio of TC50 over EC50) was calculated accordingly. As shown in FIG. 21, Compounds 64, 25, and 57 exhibited anti-viral effects with SI ranging from 10-79.

Although the present disclosure has been described in considerable detail with reference to certain preferred versions thereof, other versions are possible. Therefore, the spirit and scope of the present disclosure should not be limited to the description of the preferred versions described herein.

Although compositions, materials, and methods similar or equivalent to those described herein can be used in the practice or testing of the present invention, suitable preparations, methods and materials are described herein. All publications mentioned herein are incorporated by reference in their entirety. In the case of conflict, the present specification, including definitions, will control. In addition, the particular embodiments discussed below are illustrative only and not intended to be limiting.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the present disclosure, in addition to those described herein, will be apparent to those skilled in the art from the foregoing description. Such modifications are also intended to fall within the scope of the appended claims.

1.-22. (canceled)

23. A compound of Formula Ia 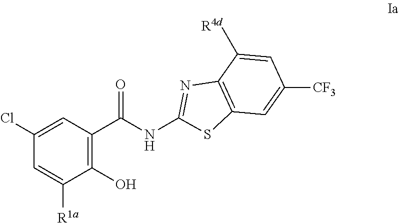
wherein R^{1a} is selected from the group consisting of —CH₃, —CH₂CH₃, —(CH₂)N(CH₂CH₂OCH₃)₂, 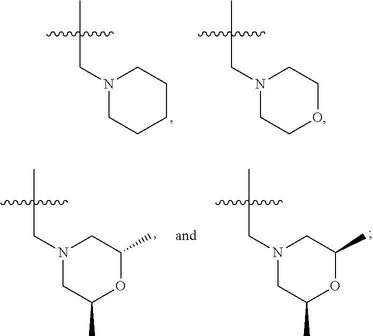
and
R^{4d} is selected from the group consisting of CH₂OH, —CH₂OCH₂CH₃, —CH₂OCH₃, —CH₂O(CH₂)₂OH, —CH₂O(CH₂)₂OCH₃, —CH₂O(CH₂)₂NHCH₃, —CH₂O(CH₂)₂N(CH₃)₂, —CH₂O(CH₂)₂NHSO₂CH₃, 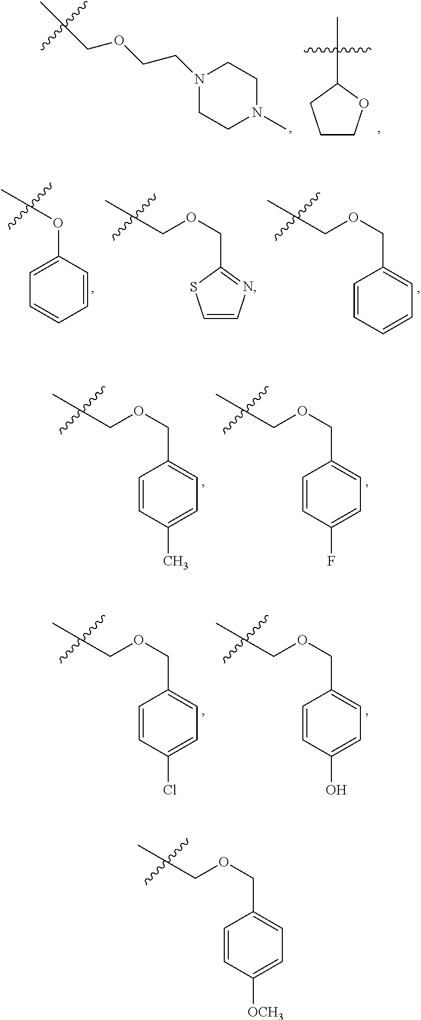
—OCH₃;
or a pharmaceutically acceptable salt, solvate, or prodrug thereof.

24-38. (canceled)

39. A compound of Formula IIa 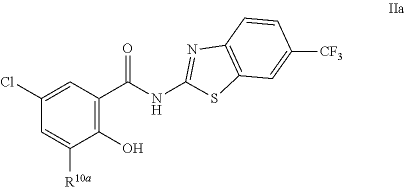
wherein
R^{10a} is selected from the group consisting of —OCH₃, —CH₂OCH₃, —CH₂OCH₂OCH₃, —CH₂O(CH₂)₂OH, —CH₂O(CH₂)₂OCH₃, —(CH₂)NH(CH₂)₂OCH₃, —CH₂NHC(O)CH₃, 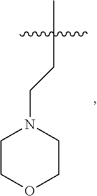 [SMILES: *CCN1CCOCC1]
CH₂N(CH₃)(CH₂)₂OCH₃, —(CH₂)N(CH₂CH₂OCH₃)₂, —CH₂N(CH₃)(CH₂)₂O(CH₂)₂OCH₃, 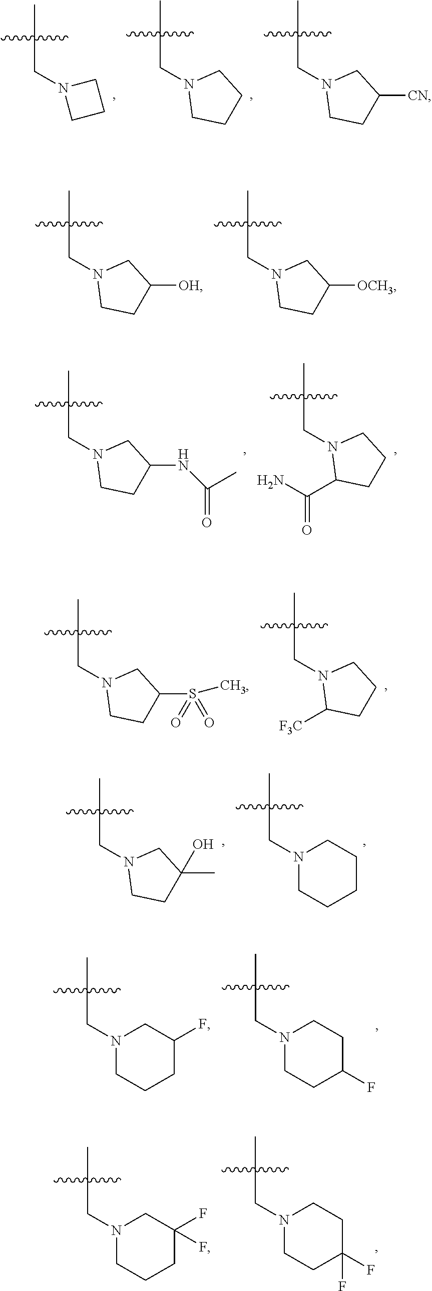 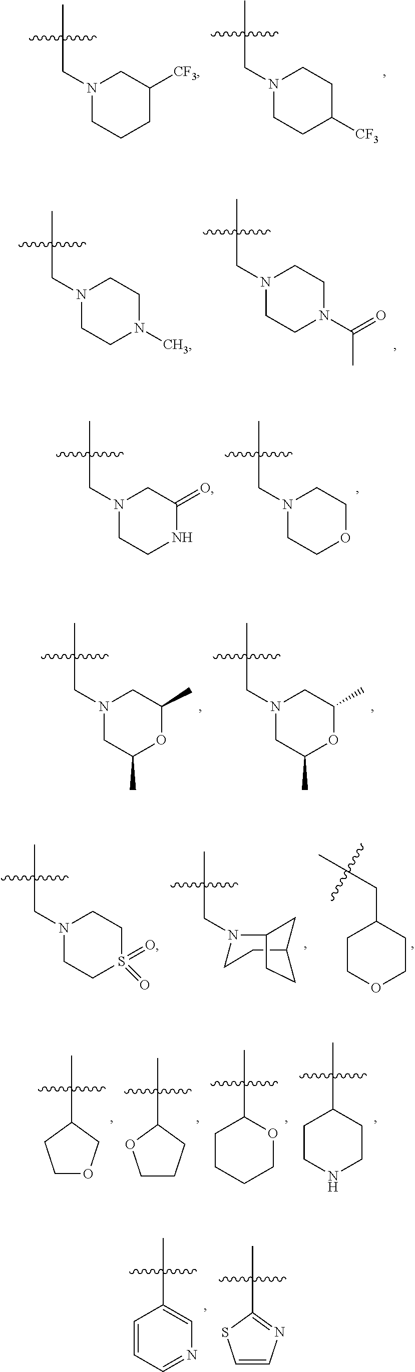
and C(O)N(CH₂CH₂OCH₃)₂;
or a pharmaceutically acceptable salt, solvate, or prodrug thereof.

40.-41. (canceled)

42. The compound of claim 39 that is

| | |
|---|---|
| 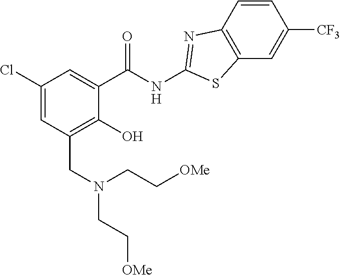 [SMILES: COCCN(CCOC)Cc1cc(Cl)cc(C(=O)Nc2nc3ccc(C(F)(F)F)cc3s2)c1O] | 3-((bis(2-methoxyethyl)amino)methyl)-5- chloro-2-hydroxy-N-(6- (trifluoromethyl)benzo[d]thiazol-2- yl)benzamide 3-((bis(2-methoxyethyl)amino)methyl)-5- chloro-2-hydroxy-N-(6- (trifluoromethyl)benzo[d]thiazol-2- yl)benzamide hydrochloride |

or a pharmaceutically acceptable salt, solvate, or prodrug thereof.

43-61. (canceled)

62. A compound of Formula IIIa 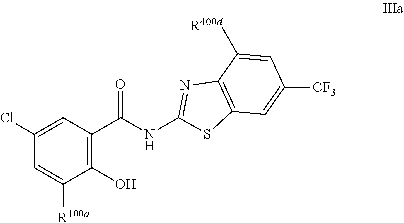
wherein R^{100a} is selected from the group consisting of —CH₃, —(CH₂)N(CH₂CH₂OCH₃)₂, CH₃, 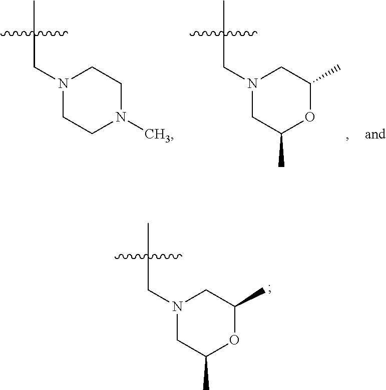
and
R^{400d} is selected from the group consisting of —CH₂NHC(O)CH₂CH₃, —CH₂NHC(O)CH₂OCH₃, —CH₂NHSO₂CH₃, —CH₂N(CH₃)₂ 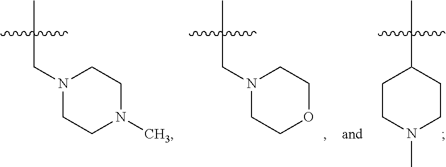
or a pharmaceutically acceptable salt, solvate, or prodrug thereof.

63. The compound of claim 62 selected from the group consisting of

| | |
|---|---|
| 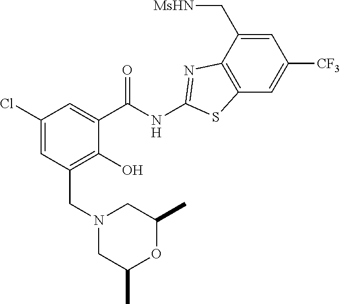 | 5-chloro-3-((cis-2,6- dimethylmorpholino)methyl)-2-hydroxy-N-(4- (methylsulfonamidomethyl)-6- (trifluoromethyl)benzo[d]thiazol-2- yl)benzamide |
| 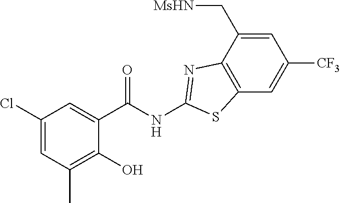 | 5-chloro-2-hydroxy-3-methyl-N-(4- (methylsulfonamidomethyl)-6- (trifluoromethyl)benzo[d]thiazol-2- yl)benzamide |
| 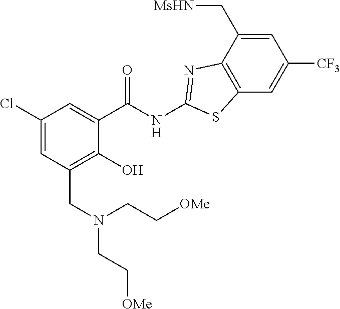 | 3-((bis(2-methoxyethyl)amino)methyl)-5- chloro-2-hydroxy-N-(4- (methylsulfonamidomethyl)-6- (trifluoromethyl)benzo[d]thiazol-2- yl)benzamid |
| 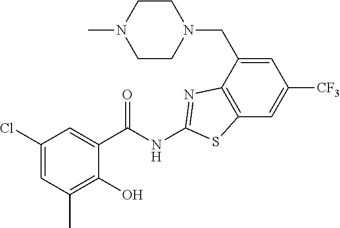 [SMILES: Cc1cc(Cl)cc(C(=O)Nc2nc3c(CN4CCN(C)CC4)cc(C(F)(F)F)cc3s2)c1O] | 5-chloro-2-hydroxy-3-methyl-N-(4-((4- methylpiperazin-1-yl)methyl)-6- (trifluoromethyl)benzo[d]thiazol-2- yl)benzamide |

or a pharmaceutically acceptable salt, solvate, or prodrug thereof.

64. A pharmaceutical composition comprising a compound of claim 23, or a pharmaceutically acceptable salt or prodrug thereof and a pharmaceutically acceptable carrier or diluent.

65. A method of treating a mitochondria-related condition or disorder in a subject in need thereof, comprising administering to the subject a therapeutically effective amount of a pharmaceutical composition according to claim 64.

66. The method of claim 65 wherein the mitochondria-related condition or disorder has and one or more underlying causal factors or one or more underlying symptoms wherein the one or more underlying causal factors or one or more underlying symptoms is selected from the group consisting of hyperglycemia, abnormal accumulation of lipid in cells, abnormal accumulation of lipid in tissues, abnormal lipid metabolism, abnormal mitochondrial metabolism, insulin resistance, abnormal cell proliferation, abnormal TGF-beta activation, and abnormal fibrosis.

67. (canceled)

68. The method of claim 65, wherein the mitochondria-related condition or disorder is selected from the group consisting of a metabolic disease, cancer, an autoimmune disease, pulmonary fibrosis, a dermatological disorder, an infectious disease, and a neurodegenerative disease.

69.-75. (canceled)

76. The method of claim 68, wherein the pharmaceutical composition is administered in combination with a second agent.

77.-97. (canceled)

98. A method of preparing a mitochondrial membrane-retaining mitochondrial uncoupler comprising:
1. identifying a conventional mitochondrial uncoupler;
2. designing a compound that covalently links at least one secondary or tertiary amino moiety to a conventional mitochondrial uncoupler; and
3. preparing the compound of step 2, wherein the compound is a mitochondrial membrane retaining uncoupler compound.

99. A mitochondrial membrane-retaining uncoupler compound of the Formula: 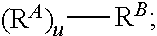
or a pharmaceutically acceptable salt, solvate or prodrug thereof;
wherein R^{A} and R^{B} are covalently linked;
each R^{A} is independently a moiety containing a secondary or tertiary amine;
u is an integer selected from the group consisting of 1 and 2; and
R^{B} is a conventional mitochondrial uncoupler prior to being covalently linked to R^{A};
provided the mitochondrial membrane-retaining uncoupler compound is not

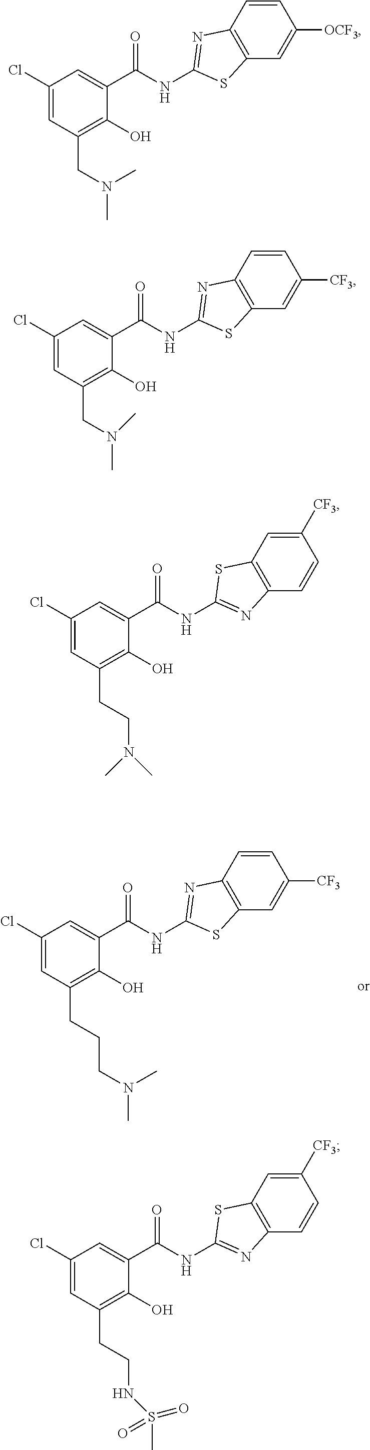 [SMILES: CN(C)Cc1cc(Cl)cc(C(=O)Nc2nc3ccc(OC(F)(F)F)cc3s2)c1O ; CN(C)Cc1cc(Cl)cc(C(=O)Nc2nc3ccc(C(F)(F)F)cc3s2)c1O ; CN(C)CCc1cc(Cl)cc(C(=O)Nc2nc3ccc(C(F)(F)F)cc3s2)c1O ; CN(C)CCCc1cc(Cl)cc(C(=O)Nc2nc3ccc(C(F)(F)F)cc3s2)c1O ; CS(=O)(=O)NCCc1cc(Cl)cc(C(=O)Nc2nc3ccc(C(F)(F)F)cc3s2)c1O]

100. The mitochondrial membrane-retaining uncoupler compound of claim 99 wherein R^{A} is selected from the group consisting of —CH₂NHSO₂CH₃, —CH₂N(CH₃)₂, —(CH₂)₂N(CH₃)₂, —(CH₂)NH(CH₂)₂OCH₃, —CH₂NHC(O)CH₃, —CH₂NHC(O)CH₂CH₃, 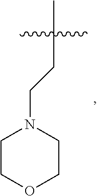 [SMILES: *CCN1CCOCC1]
—CH₂N(CH₃)(CH₂)₂OCH₃, —(CH₂)N(CH₂CH₂OCH₃)₂, —CH₂N(CH₃)(CH₂)₂O(CH₂)₂OCH₃, 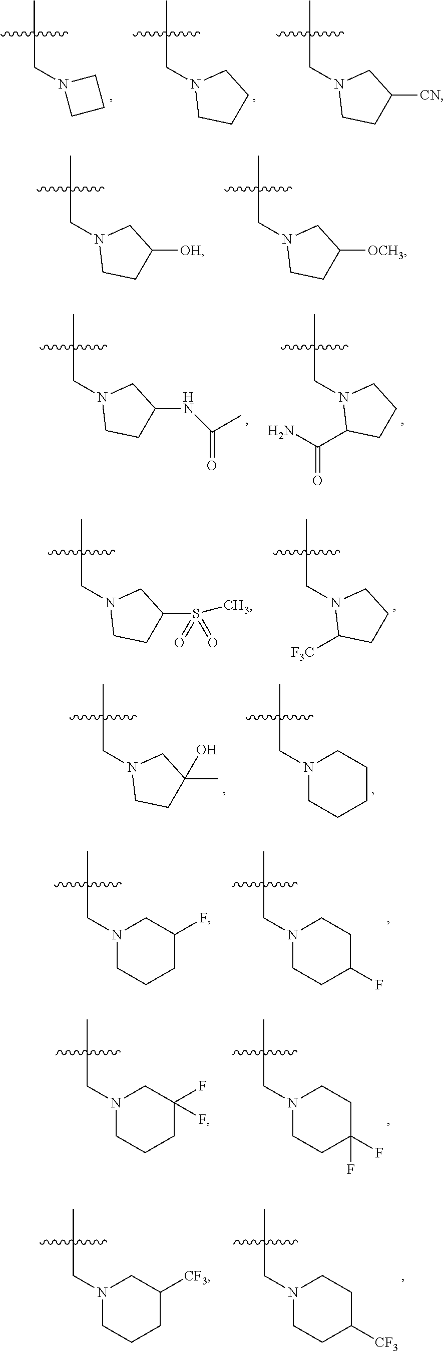 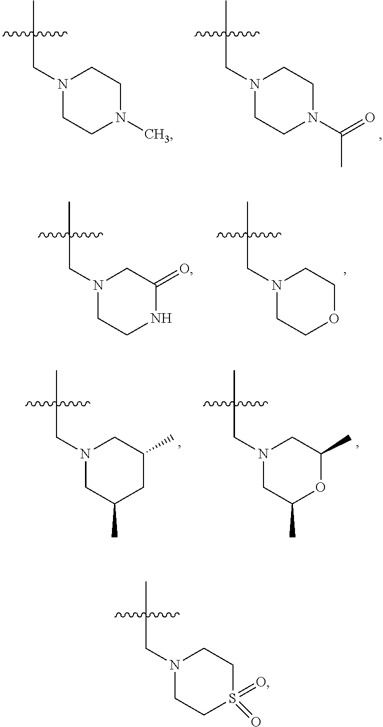
C(O)N(CH₂CH₂OCH₃)₂, —CH₂O(CH₂)₂N(CH₃)₂, —CH₂O(CH₂)₂NHSO₂CH₃,

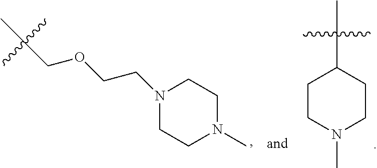

101. A pharmaceutical composition comprising a compound of claim 39, or a pharmaceutically acceptable salt or prodrug thereof and a pharmaceutically acceptable carrier or diluent.

102. A method of treating a mitochondria-related condition or disorder in a subject in need thereof, comprising administering to the subject a therapeutically effective amount of a pharmaceutical composition according to claim 101.

103. The method of claim 102 wherein the mitochondria-related condition or disorder has and one or more underlying causal factors or one or more underlying symptoms wherein the one or more underlying causal factors or one or more underlying symptoms is selected from the group consisting of hyperglycemia, abnormal accumulation of lipid in cells, abnormal accumulation of lipid in tissues, abnormal lipid metabolism, abnormal mitochondrial metabolism, insulin resistance, abnormal cell proliferation, abnormal TGF-beta activation, and abnormal fibrosis.

104. The method of claim 102, wherein the mitochondria-related condition or disorder is selected from the group consisting of a metabolic disease, cancer, an autoimmune disease, pulmonary fibrosis, a dermatological disorder, an infectious disease, and a neurodegenerative disease.

105. The method of claim 102, wherein the pharmaceutical composition is administered in combination with a second agent.

106. A pharmaceutical composition comprising a compound of claim 62 or a pharmaceutically acceptable salt or prodrug thereof and a pharmaceutically acceptable carrier or diluent.

107. A method of treating a mitochondria-related condition or disorder in a subject in need thereof, comprising administering to the subject a therapeutically effective amount of a pharmaceutical composition according to claim 106.

108. The method of claim 107 wherein the mitochondria-related condition or disorder has and one or more underlying causal factors or one or more underlying symptoms wherein the one or more underlying causal factors or one or more underlying symptoms is selected from the group consisting of hyperglycemia, abnormal accumulation of lipid in cells, abnormal accumulation of lipid in tissues, abnormal lipid metabolism, abnormal mitochondrial metabolism, insulin resistance, abnormal cell proliferation, abnormal TGF-beta activation, and abnormal fibrosis.

109. The method of claim 107, wherein the mitochondria-related condition or disorder is selected from the group consisting of a metabolic disease, cancer, an autoimmune disease, pulmonary fibrosis, a dermatological disorder, an infectious disease, and a neurodegenerative disease.

110. The method of claim 102, wherein the pharmaceutical composition is administered in combination with a second agent.
